SEQUENCE LISTING (357 sequences)

SEQ ID NO: 1 - DNA, 38 bp, Artificial Sequence
  Primer
ctcgatgctacgtgactactgcgtcgaggagtctatcc

SEQ ID NO: 2 - DNA, 96 bp, Artificial Sequence
  Primer
atacctgactgctcgtcagttgagcgaattccgtatgggtagcaaggtccaagagaggctccatcctcactcgcctgactacgacaagacctactg

SEQ ID NO: 3 - DNA, 98 bp, Artificial Sequence
  Primer
tccagtaggtcttgtcgtagtcaggcgagtgaggatggagcctctcttggaccttgctacccatacggaattcgctcaactgacgagcagtcaggtat

SEQ ID NO: 4 - DNA, 22 bp, Artificial Sequence
  Primer
ctcgatgctacgtgactactgc

SEQ ID NO: 5 - DNA, 22 bp, Artificial Sequence
  Primer
cagtaggtcttgtcgtagtcag

SEQ ID NO: 6 - DNA, 45 bp, Artificial Sequence
  Primer
atggtacacacctacactactcggacgctcttccgatcttgacct

SEQ ID NO: 7 - DNA, 44 bp, Artificial Sequence
  Primer
aggtcaagatcggaagagcgtccgagtagtgtaggtgtgtacca

SEQ ID NO: 8 - DNA, 22 bp, Artificial Sequence
  Primer
tggtacacacctacactactcg

SEQ ID NO: 9 - DNA, 20 bp, Artificial Sequence
  Primer
ccatacggaattcgctcaac

SEQ ID NO: 10 - DNA, 42 bp, Artificial Sequence
  Primer
gttgagcgaattccgtatggtggtacacacctacactactcg

SEQ ID NO: 11 - DNA, 46 bp, Artificial Sequence
  Primer
taggacgatacgagtgtgtactcgtggtacacacctacactactcg

SEQ ID NO: 12 - DNA, 42 bp, Artificial Sequence
  Primer
cgagtagtgtaggtgtgtaccaccatacggaattcgctcaac

SEQ ID NO: 13 - DNA, 44 bp, Artificial Sequence
  Primer
ctgtcaaggtagactagcatgctcccatacggaattcgctcaac

SEQ ID NO: 14 - DNA, 24 bp, Artificial Sequence
  Primer
taggacgatacgagtgtgtactcg

SEQ ID NO: 15 - DNA, 24 bp, Artificial Sequence
  Primer
ctgtcaaggtagactagcatgctc

SEQ ID NO: 16 - DNA, 56 bp, Artificial Sequence
  Primer
ctcggcattcctgctgaaccgctcttccgatctgctcaactgacgagcagtcaggt

SEQ ID NO: 17 - DNA, 39 bp, Artificial Sequence
  Primer
acactctttccctacacgacgctcttccgatcttgacct

SEQ ID NO: 18 - DNA, 20 bp, Artificial Sequence
  Sequence used in the synthesis of sub-barcode molecule library
aaacggatagactcctcgac

SEQ ID NO: 19 - DNA, 20 bp, Artificial Sequence
  Sequence used in the synthesis of sub-barcode molecule library
aaagggatagactcctcgac

SEQ ID NO: 20 - DNA, 20 bp, Artificial Sequence
  Sequence used in the synthesis of sub-barcode molecule library
aaatggatagactcctcgac

SEQ ID NO: 21 - DNA, 20 bp, Artificial Sequence
  Sequence used in the synthesis of sub-barcode molecule library
aacaggatagactcctcgac

SEQ ID NO: 22 - DNA, 20 bp, Artificial Sequence
  Sequence used in the synthesis of sub-barcode molecule library
aaccggatagactcctcgac

SEQ ID NO: 23 - DNA, 20 bp, Artificial Sequence
  Sequence used in the synthesis of sub-barcode molecule library
aacgggatagactcctcgac

SEQ ID NO: 24 - DNA, 20 bp, Artificial Sequence
  Sequence used in the synthesis of sub-barcode molecule library
aactggatagactcctcgac

SEQ ID NO: 25 - DNA, 20 bp, Artificial Sequence
  Sequence used in the synthesis of sub-barcode molecule library
aagaggatagactcctcgac

SEQ ID NO: 26 - DNA, 20 bp, Artificial Sequence
  Sequence used in the synthesis of sub-barcode molecule library
aagcggatagactcctcgac

SEQ ID NO: 27 - DNA, 20 bp, Artificial Sequence
  Sequence used in the synthesis of sub-barcode molecule library
aaggggatagactcctcgac

SEQ ID NO: 28 - DNA, 20 bp, Artificial Sequence
  Sequence used in the synthesis of sub-barcode molecule library
aagtggatagactcctcgac

SEQ ID NO: 29 - DNA, 20 bp, Artificial Sequence
  Sequence used in the synthesis of sub-barcode molecule library
aataggatagactcctcgac

SEQ ID NO: 30 - DNA, 20 bp, Artificial Sequence
  Sequence used in the synthesis of sub-barcode molecule library
aatcggatagactcctcgac

SEQ ID NO: 31 - DNA, 20 bp, Artificial Sequence
  Sequence used in the synthesis of sub-barcode molecule library
aatgggatagactcctcgac

SEQ ID NO: 32 - DNA, 20 bp, Artificial Sequence
  Sequence used in the synthesis of sub-barcode molecule library
aattggatagactcctcgac

SEQ ID NO: 33 - DNA, 20 bp, Artificial Sequence
  Sequence used in the synthesis of sub-barcode molecule library
acaaggatagactcctcgac

SEQ ID NO: 34 - DNA, 20 bp, Artificial Sequence
  Sequence used in the synthesis of sub-barcode molecule library
acacggatagactcctcgac

SEQ ID NO: 35 - DNA, 20 bp, Artificial Sequence
  Sequence used in the synthesis of sub-barcode molecule library
acagggatagactcctcgac

SEQ ID NO: 36 - DNA, 20 bp, Artificial Sequence
  Sequence used in the synthesis of sub-barcode molecule library
acatggatagactcctcgac

SEQ ID NO: 37 - DNA, 20 bp, Artificial Sequence
  Sequence used in the synthesis of sub-barcode molecule library
accaggatagactcctcgac

SEQ ID NO: 38 - DNA, 20 bp, Artificial Sequence
  Sequence used in the synthesis of sub-barcode molecule library
acccggatagactcctcgac

SEQ ID NO: 39 - DNA, 20 bp, Artificial Sequence
  Sequence used in the synthesis of sub-barcode molecule library
accgggatagactcctcgac

SEQ ID NO: 40 - DNA, 20 bp, Artificial Sequence
  Sequence used in the synthesis of sub-barcode molecule library
acctggatagactcctcgac

SEQ ID NO: 41 - DNA, 20 bp, Artificial Sequence
  Sequence used in the synthesis of sub-barcode molecule library
acgaggatagactcctcgac

SEQ ID NO: 42 - DNA, 20 bp, Artificial Sequence
  Sequence used in the synthesis of sub-barcode molecule library
acgcggatagactcctcgac

SEQ ID NO: 43 - DNA, 20 bp, Artificial Sequence
  Sequence used in the synthesis of sub-barcode molecule library
acggggatagactcctcgac

SEQ ID NO: 44 - DNA, 20 bp, Artificial Sequence
  Sequence used in the synthesis of sub-barcode molecule library
acgtggatagactcctcgac

SEQ ID NO: 45 - DNA, 20 bp, Artificial Sequence
  Sequence used in the synthesis of sub-barcode molecule library
actaggatagactcctcgac

SEQ ID NO: 46 - DNA, 20 bp, Artificial Sequence
  Sequence used in the synthesis of sub-barcode molecule library
actcggatagactcctcgac

SEQ ID NO: 47 - DNA, 20 bp, Artificial Sequence
  Sequence used in the synthesis of sub-barcode molecule library
actgggatagactcctcgac

SEQ ID NO: 48 - DNA, 20 bp, Artificial Sequence
  Sequence used in the synthesis of sub-barcode molecule library
acttggatagactcctcgac

SEQ ID NO: 49 - DNA, 20 bp, Artificial Sequence
  Sequence used in the synthesis of sub-barcode molecule library
agaaggatagactcctcgac

SEQ ID NO: 50 - DNA, 20 bp, Artificial Sequence
  Sequence used in the synthesis of sub-barcode molecule library
agacggatagactcctcgac

SEQ ID NO: 51 - DNA, 20 bp, Artificial Sequence
  Sequence used in the synthesis of sub-barcode molecule library
agagggatagactcctcgac

SEQ ID NO: 52 - DNA, 20 bp, Artificial Sequence
  Sequence used in the synthesis of sub-barcode molecule library
agatggatagactcctcgac

SEQ ID NO: 53 - DNA, 20 bp, Artificial Sequence
  Sequence used in the synthesis of sub-barcode molecule library
agcaggatagactcctcgac

SEQ ID NO: 54 - DNA, 20 bp, Artificial Sequence
  Sequence used in the synthesis of sub-barcode molecule library
agccggatagactcctcgac

SEQ ID NO: 55 - DNA, 20 bp, Artificial Sequence
  Sequence used in the synthesis of sub-barcode molecule library
agcgggatagactcctcgac

SEQ ID NO: 56 - DNA, 20 bp, Artificial Sequence
  Sequence used in the synthesis of sub-barcode molecule library
agctggatagactcctcgac

SEQ ID NO: 57 - DNA, 20 bp, Artificial Sequence
  Sequence used in the synthesis of sub-barcode molecule library
aggaggatagactcctcgac

SEQ ID NO: 58 - DNA, 20 bp, Artificial Sequence
  Sequence used in the synthesis of sub-barcode molecule library
aggcggatagactcctcgac

SEQ ID NO: 59 - DNA, 20 bp, Artificial Sequence
  Sequence used in the synthesis of sub-barcode molecule library
agggggatagactcctcgac

SEQ ID NO: 60 - DNA, 20 bp, Artificial Sequence
  Sequence used in the synthesis of sub-barcode molecule library
aggtggatagactcctcgac

SEQ ID NO: 61 - DNA, 20 bp, Artificial Sequence
  Sequence used in the synthesis of sub-barcode molecule library
agtaggatagactcctcgac

SEQ ID NO: 62 - DNA, 20 bp, Artificial Sequence
  Sequence used in the synthesis of sub-barcode molecule library
agtcggatagactcctcgac

SEQ ID NO: 63 - DNA, 20 bp, Artificial Sequence
  Sequence used in the synthesis of sub-barcode molecule library
agtgggatagactcctcgac

SEQ ID NO: 64 - DNA, 20 bp, Artificial Sequence
  Sequence used in the synthesis of sub-barcode molecule library
agttggatagactcctcgac

SEQ ID NO: 65 - DNA, 20 bp, Artificial Sequence
  Sequence used in the synthesis of sub-barcode molecule library
ataaggatagactcctcgac

SEQ ID NO: 66 - DNA, 20 bp, Artificial Sequence
  Sequence used in the synthesis of sub-barcode molecule library
atacggatagactcctcgac

SEQ ID NO: 67 - DNA, 20 bp, Artificial Sequence
  Sequence used in the synthesis of sub-barcode molecule library
atagggatagactcctcgac

SEQ ID NO: 68 - DNA, 20 bp, Artificial Sequence
  Sequence used in the synthesis of sub-barcode molecule library
atatggatagactcctcgac

SEQ ID NO: 69 - DNA, 20 bp, Artificial Sequence
  Sequence used in the synthesis of sub-barcode molecule library
atcaggatagactcctcgac

SEQ ID NO: 70 - DNA, 20 bp, Artificial Sequence
  Sequence used in the synthesis of sub-barcode molecule library
atccggatagactcctcgac

SEQ ID NO: 71 - DNA, 20 bp, Artificial Sequence
  Sequence used in the synthesis of sub-barcode molecule library
atcgggatagactcctcgac

SEQ ID NO: 72 - DNA, 20 bp, Artificial Sequence
  Sequence used in the synthesis of sub-barcode molecule library
atctggatagactcctcgac

SEQ ID NO: 73 - DNA, 20 bp, Artificial Sequence
  Sequence used in the synthesis of sub-barcode molecule library
atgaggatagactcctcgac

SEQ ID NO: 74 - DNA, 20 bp, Artificial Sequence
  Sequence used in the synthesis of sub-barcode molecule library
atgcggatagactcctcgac

SEQ ID NO: 75 - DNA, 20 bp, Artificial Sequence
  Sequence used in the synthesis of sub-barcode molecule library
atggggatagactcctcgac

SEQ ID NO: 76 - DNA, 20 bp, Artificial Sequence
  Sequence used in the synthesis of sub-barcode molecule library
atgtggatagactcctcgac

SEQ ID NO: 77 - DNA, 20 bp, Artificial Sequence
  Sequence used in the synthesis of sub-barcode molecule library
attaggatagactcctcgac

SEQ ID NO: 78 - DNA, 20 bp, Artificial Sequence
  Sequence used in the synthesis of sub-barcode molecule library
attcggatagactcctcgac

SEQ ID NO: 79 - DNA, 20 bp, Artificial Sequence
  Sequence used in the synthesis of sub-barcode molecule library
attgggatagactcctcgac

SEQ ID NO: 80 - DNA, 20 bp, Artificial Sequence
  Sequence used in the synthesis of sub-barcode molecule library
atttggatagactcctcgac

SEQ ID NO: 81 - DNA, 20 bp, Artificial Sequence
  Sequence used in the synthesis of sub-barcode molecule library
caaaggatagactcctcgac

SEQ ID NO: 82 - DNA, 20 bp, Artificial Sequence
  Sequence used in the synthesis of sub-barcode molecule library
caacggatagactcctcgac

SEQ ID NO: 83 - DNA, 20 bp, Artificial Sequence
  Sequence used in the synthesis of sub-barcode molecule library
caagggatagactcctcgac

SEQ ID NO: 84 - DNA, 20 bp, Artificial Sequence
  Sequence used in the synthesis of sub-barcode molecule library
caatggatagactcctcgac

SEQ ID NO: 85 - DNA, 20 bp, Artificial Sequence
  Sequence used in the synthesis of sub-barcode molecule library
cacaggatagactcctcgac

SEQ ID NO: 86 - DNA, 20 bp, Artificial Sequence
  Sequence used in the synthesis of sub-barcode molecule library
caccggatagactcctcgac

SEQ ID NO: 87 - DNA, 20 bp, Artificial Sequence
  Sequence used in the synthesis of sub-barcode molecule library
cacgggatagactcctcgac

SEQ ID NO: 88 - DNA, 20 bp, Artificial Sequence
  Sequence used in the synthesis of sub-barcode molecule library
cactggatagactcctcgac

SEQ ID NO: 89 - DNA, 20 bp, Artificial Sequence
  Sequence used in the synthesis of sub-barcode molecule library
cagaggatagactcctcgac

SEQ ID NO: 90 - DNA, 20 bp, Artificial Sequence
  Sequence used in the synthesis of sub-barcode molecule library
cagcggatagactcctcgac

SEQ ID NO: 91 - DNA, 20 bp, Artificial Sequence
  Sequence used in the synthesis of sub-barcode molecule library
caggggatagactcctcgac

SEQ ID NO: 92 - DNA, 20 bp, Artificial Sequence
  Sequence used in the synthesis of sub-barcode molecule library
cagtggatagactcctcgac

SEQ ID NO: 93 - DNA, 20 bp, Artificial Sequence
  Sequence used in the synthesis of sub-barcode molecule library
cataggatagactcctcgac

SEQ ID NO: 94 - DNA, 20 bp, Artificial Sequence
  Sequence used in the synthesis of sub-barcode molecule library
catcggatagactcctcgac

SEQ ID NO: 95 - DNA, 20 bp, Artificial Sequence
  Sequence used in the synthesis of sub-barcode molecule library
catgggatagactcctcgac

SEQ ID NO: 96 - DNA, 20 bp, Artificial Sequence
  Sequence used in the synthesis of sub-barcode molecule library
cattggatagactcctcgac

SEQ ID NO: 97 - DNA, 20 bp, Artificial Sequence
  Sequence used in the synthesis of sub-barcode molecule library
ccaaggatagactcctcgac

SEQ ID NO: 98 - DNA, 20 bp, Artificial Sequence
  Sequence used in the synthesis of sub-barcode molecule library
ccacggatagactcctcgac

SEQ ID NO: 99 - DNA, 20 bp, Artificial Sequence
  Sequence used in the synthesis of sub-barcode molecule library
ccagggatagactcctcgac

SEQ ID NO: 100 - DNA, 20 bp, Artificial Sequence
  Sequence used in the synthesis of sub-barcode molecule library
ccatggatagactcctcgac

SEQ ID NO: 101 - DNA, 20 bp, Artificial Sequence
  Sequence used in the synthesis of sub-barcode molecule library
cccaggatagactcctcgac

SEQ ID NO: 102 - DNA, 20 bp, Artificial Sequence
  Sequence used in the synthesis of sub-barcode molecule library
cccgggatagactcctcgac

SEQ ID NO: 103 - DNA, 20 bp, Artificial Sequence
  Sequence used in the synthesis of sub-barcode molecule library
ccctggatagactcctcgac

SEQ ID NO: 104 - DNA, 20 bp, Artificial Sequence
  Sequence used in the synthesis of sub-barcode molecule library
ccgaggatagactcctcgac

SEQ ID NO: 105 - DNA, 20 bp, Artificial Sequence
  Sequence used in the synthesis of sub-barcode molecule library
ccgcggatagactcctcgac

SEQ ID NO: 106 - DNA, 20 bp, Artificial Sequence
  Sequence used in the synthesis of sub-barcode molecule library
ccggggatagactcctcgac

SEQ ID NO: 107 - DNA, 20 bp, Artificial Sequence
  Sequence used in the synthesis of sub-barcode molecule library
ccgtggatagactcctcgac

SEQ ID NO: 108 - DNA, 20 bp, Artificial Sequence
  Sequence used in the synthesis of sub-barcode molecule library
cctaggatagactcctcgac

SEQ ID NO: 109 - DNA, 20 bp, Artificial Sequence
  Sequence used in the synthesis of sub-barcode molecule library
cctcggatagactcctcgac

SEQ ID NO: 110 - DNA, 20 bp, Artificial Sequence
  Sequence used in the synthesis of sub-barcode molecule library
cctgggatagactcctcgac

SEQ ID NO: 111 - DNA, 20 bp, Artificial Sequence
  Sequence used in the synthesis of sub-barcode molecule library
ccttggatagactcctcgac

SEQ ID NO: 112 - DNA, 20 bp, Artificial Sequence
  Sequence used in the synthesis of sub-barcode molecule library
cgaaggatagactcctcgac

SEQ ID NO: 113 - DNA, 20 bp, Artificial Sequence
  Sequence used in the synthesis of sub-barcode molecule library
cgacggatagactcctcgac

SEQ ID NO: 114 - DNA, 20 bp, Artificial Sequence
  Sequence used in the synthesis of sub-barcode molecule library
cgagggatagactcctcgac

SEQ ID NO: 115 - DNA, 20 bp, Artificial Sequence
  Sequence used in the synthesis of sub-barcode molecule library
cgatggatagactcctcgac

SEQ ID NO: 116 - DNA, 20 bp, Artificial Sequence
  Sequence used in the synthesis of sub-barcode molecule library
cgcaggatagactcctcgac

SEQ ID NO: 117 - DNA, 20 bp, Artificial Sequence
  Sequence used in the synthesis of sub-barcode molecule library
cgccggatagactcctcgac

SEQ ID NO: 118 - DNA, 20 bp, Artificial Sequence
  Sequence used in the synthesis of sub-barcode molecule library
cgcgggatagactcctcgac

SEQ ID NO: 119 - DNA, 20 bp, Artificial Sequence
  Sequence used in the synthesis of sub-barcode molecule library
cgctggatagactcctcgac

SEQ ID NO: 120 - DNA, 20 bp, Artificial Sequence
  Sequence used in the synthesis of sub-barcode molecule library
cggaggatagactcctcgac

SEQ ID NO: 121 - DNA, 20 bp, Artificial Sequence
  Sequence used in the synthesis of sub-barcode molecule library
cggcggatagactcctcgac

SEQ ID NO: 122 - DNA, 20 bp, Artificial Sequence
  Sequence used in the synthesis of sub-barcode molecule library
cgggggatagactcctcgac

SEQ ID NO: 123 - DNA, 20 bp, Artificial Sequence
  Sequence used in the synthesis of sub-barcode molecule library
cggtggatagactcctcgac

SEQ ID NO: 124 - DNA, 20 bp, Artificial Sequence
  Sequence used in the synthesis of sub-barcode molecule library
cgtaggatagactcctcgac

SEQ ID NO: 125 - DNA, 20 bp, Artificial Sequence
  Sequence used in the synthesis of sub-barcode molecule library
cgtcggatagactcctcgac

SEQ ID NO: 126 - DNA, 20 bp, Artificial Sequence
  Sequence used in the synthesis of sub-barcode molecule library
cgtgggatagactcctcgac

SEQ ID NO: 127 - DNA, 20 bp, Artificial Sequence
  Sequence used in the synthesis of sub-barcode molecule library
cgttggatagactcctcgac

SEQ ID NO: 128 - DNA, 20 bp, Artificial Sequence
  Sequence used in the synthesis of sub-barcode molecule library
ctaaggatagactcctcgac

SEQ ID NO: 129 - DNA, 20 bp, Artificial Sequence
  Sequence used in the synthesis of sub-barcode molecule library
ctacggatagactcctcgac

SEQ ID NO: 130 - DNA, 20 bp, Artificial Sequence
  Sequence used in the synthesis of sub-barcode molecule library
ctagggatagactcctcgac

SEQ ID NO: 131 - DNA, 20 bp, Artificial Sequence
  Sequence used in the synthesis of sub-barcode molecule library
ctatggatagactcctcgac

SEQ ID NO: 132 - DNA, 20 bp, Artificial Sequence
  Sequence used in the synthesis of sub-barcode molecule library
ctcaggatagactcctcgac

SEQ ID NO: 133 - DNA, 20 bp, Artificial Sequence
  Sequence used in the synthesis of sub-barcode molecule library
ctccggatagactcctcgac

SEQ ID NO: 134 - DNA, 20 bp, Artificial Sequence
  Sequence used in the synthesis of sub-barcode molecule library
ctcgggatagactcctcgac

SEQ ID NO: 135 - DNA, 20 bp, Artificial Sequence
  Sequence used in the synthesis of sub-barcode molecule library
ctctggatagactcctcgac

SEQ ID NO: 136 - DNA, 20 bp, Artificial Sequence
  Sequence used in the synthesis of sub-barcode molecule library
ctgaggatagactcctcgac

SEQ ID NO: 137 - DNA, 20 bp, Artificial Sequence
  Sequence used in the synthesis of sub-barcode molecule library
ctgcggatagactcctcgac

SEQ ID NO: 138 - DNA, 20 bp, Artificial Sequence
  Sequence used in the synthesis of sub-barcode molecule library
ctggggatagactcctcgac

SEQ ID NO: 139 - DNA, 20 bp, Artificial Sequence
  Sequence used in the synthesis of sub-barcode molecule library
ctgtggatagactcctcgac

SEQ ID NO: 140 - DNA, 20 bp, Artificial Sequence
  Sequence used in the synthesis of sub-barcode molecule library
cttaggatagactcctcgac

SEQ ID NO: 141 - DNA, 20 bp, Artificial Sequence
  Sequence used in the synthesis of sub-barcode molecule library
cttcggatagactcctcgac

SEQ ID NO: 142 - DNA, 20 bp, Artificial Sequence
  Sequence used in the synthesis of sub-barcode molecule library
cttgggatagactcctcgac

SEQ ID NO: 143 - DNA, 20 bp, Artificial Sequence
  Sequence used in the synthesis of sub-barcode molecule library
ctttggatagactcctcgac

SEQ ID NO: 144 - DNA, 20 bp, Artificial Sequence
  Sequence used in the synthesis of sub-barcode molecule library
gaaaggatagactcctcgac

SEQ ID NO: 145 - DNA, 20 bp, Artificial Sequence
  Sequence used in the synthesis of sub-barcode molecule library
gaacggatagactcctcgac

SEQ ID NO: 146 - DNA, 20 bp, Artificial Sequence
  Sequence used in the synthesis of sub-barcode molecule library
gaagggatagactcctcgac

SEQ ID NO: 147 - DNA, 20 bp, Artificial Sequence
  Sequence used in the synthesis of sub-barcode molecule library
gaatggatagactcctcgac

SEQ ID NO: 148 - DNA, 20 bp, Artificial Sequence
  Sequence used in the synthesis of sub-barcode molecule library
gacaggatagactcctcgac

SEQ ID NO: 149 - DNA, 20 bp, Artificial Sequence
  Sequence used in the synthesis of sub-barcode molecule library
gaccggatagactcctcgac

SEQ ID NO: 150 - DNA, 20 bp, Artificial Sequence
  Sequence used in the synthesis of sub-barcode molecule library
gacgggatagactcctcgac

SEQ ID NO: 151 - DNA, 20 bp, Artificial Sequence
  Sequence used in the synthesis of sub-barcode molecule library
gactggatagactcctcgac

SEQ ID NO: 152 - DNA, 20 bp, Artificial Sequence
  Sequence used in the synthesis of sub-barcode molecule library
gagaggatagactcctcgac

SEQ ID NO: 153 - DNA, 20 bp, Artificial Sequence
  Sequence used in the synthesis of sub-barcode molecule library
gagcggatagactcctcgac

SEQ ID NO: 154 - DNA, 20 bp, Artificial Sequence
  Sequence used in the synthesis of sub-barcode molecule library
gaggggatagactcctcgac

SEQ ID NO: 155 - DNA, 20 bp, Artificial Sequence
  Sequence used in the synthesis of sub-barcode molecule library
gagtggatagactcctcgac

SEQ ID NO: 156 - DNA, 20 bp, Artificial Sequence
  Sequence used in the synthesis of sub-barcode molecule library
gataggatagactcctcgac

SEQ ID NO: 157 - DNA, 20 bp, Artificial Sequence
  Sequence used in the synthesis of sub-barcode molecule library
gatcggatagactcctcgac

SEQ ID NO: 158 - DNA, 20 bp, Artificial Sequence
  Sequence used in the synthesis of sub-barcode molecule library
gatgggatagactcctcgac

SEQ ID NO: 159 - DNA, 20 bp, Artificial Sequence
  Sequence used in the synthesis of sub-barcode molecule library
gattggatagactcctcgac

SEQ ID NO: 160 - DNA, 20 bp, Artificial Sequence
  Sequence used in the synthesis of sub-barcode molecule library
gcaaggatagactcctcgac

SEQ ID NO: 161 - DNA, 20 bp, Artificial Sequence
  Sequence used in the synthesis of sub-barcode molecule library
gcacggatagactcctcgac

SEQ ID NO: 162 - DNA, 20 bp, Artificial Sequence
  Sequence used in the synthesis of sub-barcode molecule library
gcagggatagactcctcgac

SEQ ID NO: 163 - DNA, 20 bp, Artificial Sequence
  Sequence used in the synthesis of sub-barcode molecule library
gcatggatagactcctcgac

SEQ ID NO: 164 - DNA, 20 bp, Artificial Sequence
  Sequence used in the synthesis of sub-barcode molecule library
gccaggatagactcctcgac

SEQ ID NO: 165 - DNA, 20 bp, Artificial Sequence
  Sequence used in the synthesis of sub-barcode molecule library
gcccggatagactcctcgac

SEQ ID NO: 166 - DNA, 20 bp, Artificial Sequence
  Sequence used in the synthesis of sub-barcode molecule library
gccgggatagactcctcgac

SEQ ID NO: 167 - DNA, 20 bp, Artificial Sequence
  Sequence used in the synthesis of sub-barcode molecule library
gcctggatagactcctcgac

SEQ ID NO: 168 - DNA, 20 bp, Artificial Sequence
  Sequence used in the synthesis of sub-barcode molecule library
gcgaggatagactcctcgac

SEQ ID NO: 169 - DNA, 20 bp, Artificial Sequence
  Sequence used in the synthesis of sub-barcode molecule library
gcgcggatagactcctcgac

SEQ ID NO: 170 - DNA, 20 bp, Artificial Sequence
  Sequence used in the synthesis of sub-barcode molecule library
gcggggatagactcctcgac

SEQ ID NO: 171 - DNA, 20 bp, Artificial Sequence
  Sequence used in the synthesis of sub-barcode molecule library
gcgtggatagactcctcgac

SEQ ID NO: 172 - DNA, 20 bp, Artificial Sequence
  Sequence used in the synthesis of sub-barcode molecule library
gctaggatagactcctcgac

SEQ ID NO: 173 - DNA, 20 bp, Artificial Sequence
  Sequence used in the synthesis of sub-barcode molecule library
gctcggatagactcctcgac

SEQ ID NO: 174 - DNA, 20 bp, Artificial Sequence
  Sequence used in the synthesis of sub-barcode molecule library
gctgggatagactcctcgac

SEQ ID NO: 175 - DNA, 20 bp, Artificial Sequence
  Sequence used in the synthesis of sub-barcode molecule library
gcttggatagactcctcgac

SEQ ID NO: 176 - DNA, 20 bp, Artificial Sequence
  Sequence used in the synthesis of sub-barcode molecule library
ggaaggatagactcctcgac

SEQ ID NO: 177 - DNA, 20 bp, Artificial Sequence
  Sequence used in the synthesis of sub-barcode molecule library
ggacggatagactcctcgac

SEQ ID NO: 178 - DNA, 20 bp, Artificial Sequence
  Sequence used in the synthesis of sub-barcode molecule library
ggagggatagactcctcgac

SEQ ID NO: 179 - DNA, 20 bp, Artificial Sequence
  Sequence used in the synthesis of sub-barcode molecule library
ggatggatagactcctcgac

SEQ ID NO: 180 - DNA, 20 bp, Artificial Sequence
  Sequence used in the synthesis of sub-barcode molecule library
ggcaggatagactcctcgac

SEQ ID NO: 181 - DNA, 20 bp, Artificial Sequence
  Sequence used in the synthesis of sub-barcode molecule library
ggccggatagactcctcgac

SEQ ID NO: 182 - DNA, 20 bp, Artificial Sequence
  Sequence used in the synthesis of sub-barcode molecule library
ggcgggatagactcctcgac

SEQ ID NO: 183 - DNA, 20 bp, Artificial Sequence
  Sequence used in the synthesis of sub-barcode molecule library
ggctggatagactcctcgac

SEQ ID NO: 184 - DNA, 20 bp, Artificial Sequence
  Sequence used in the synthesis of sub-barcode molecule library
gggaggatagactcctcgac

SEQ ID NO: 185 - DNA, 20 bp, Artificial Sequence
  Sequence used in the synthesis of sub-barcode molecule library
gggcggatagactcctcgac

SEQ ID NO: 186 - DNA, 20 bp, Artificial Sequence
  Sequence used in the synthesis of sub-barcode molecule library
gggtggatagactcctcgac

SEQ ID NO: 187 - DNA, 20 bp, Artificial Sequence
  Sequence used in the synthesis of sub-barcode molecule library
ggtaggatagactcctcgac

SEQ ID NO: 188 - DNA, 20 bp, Artificial Sequence
  Sequence used in the synthesis of sub-barcode molecule library
ggtcggatagactcctcgac

SEQ ID NO: 189 - DNA, 20 bp, Artificial Sequence
  Sequence used in the synthesis of sub-barcode molecule library
ggtgggatagactcctcgac

SEQ ID NO: 190 - DNA, 20 bp, Artificial Sequence
  Sequence used in the synthesis of sub-barcode molecule library
ggttggatagactcctcgac

SEQ ID NO: 191 - DNA, 20 bp, Artificial Sequence
  Sequence used in the synthesis of sub-barcode molecule library
gtaaggatagactcctcgac

SEQ ID NO: 192 - DNA, 20 bp, Artificial Sequence
  Sequence used in the synthesis of sub-barcode molecule library
gtacggatagactcctcgac

SEQ ID NO: 193 - DNA, 20 bp, Artificial Sequence
  Sequence used in the synthesis of sub-barcode molecule library
gtagggatagactcctcgac

SEQ ID NO: 194 - DNA, 20 bp, Artificial Sequence
  Sequence used in the synthesis of sub-barcode molecule library
gtatggatagactcctcgac

SEQ ID NO: 195 - DNA, 20 bp, Artificial Sequence
  Sequence used in the synthesis of sub-barcode molecule library
gtcaggatagactcctcgac

SEQ ID NO: 196 - DNA, 20 bp, Artificial Sequence
  Sequence used in the synthesis of sub-barcode molecule library
gtccggatagactcctcgac

SEQ ID NO: 197 - DNA, 20 bp, Artificial Sequence
  Sequence used in the synthesis of sub-barcode molecule library
gtcgggatagactcctcgac

SEQ ID NO: 198 - DNA, 20 bp, Artificial Sequence
  Sequence used in the synthesis of sub-barcode molecule library
gtctggatagactcctcgac

SEQ ID NO: 199 - DNA, 20 bp, Artificial Sequence
  Sequence used in the synthesis of sub-barcode molecule library
gtgaggatagactcctcgac

SEQ ID NO: 200 - DNA, 20 bp, Artificial Sequence
  Sequence used in the synthesis of sub-barcode molecule library
gtgcggatagactcctcgac

SEQ ID NO: 201 - DNA, 20 bp, Artificial Sequence
  Sequence used in the synthesis of sub-barcode molecule library
gtggggatagactcctcgac

SEQ ID NO: 202 - DNA, 20 bp, Artificial Sequence
  Sequence used in the synthesis of sub-barcode molecule library
gtgtggatagactcctcgac

SEQ ID NO: 203 - DNA, 20 bp, Artificial Sequence
  Sequence used in the synthesis of sub-barcode molecule library
gttaggatagactcctcgac

SEQ ID NO: 204 - DNA, 20 bp, Artificial Sequence
  Sequence used in the synthesis of sub-barcode molecule library
gttcggatagactcctcgac

SEQ ID NO: 205 - DNA, 20 bp, Artificial Sequence
  Sequence used in the synthesis of sub-barcode molecule library
gttgggatagactcctcgac

SEQ ID NO: 206 - DNA, 20 bp, Artificial Sequence
  Sequence used in the synthesis of sub-barcode molecule library
gtttggatagactcctcgac

SEQ ID NO: 207 - DNA, 20 bp, Artificial Sequence
  Sequence used in the synthesis of sub-barcode molecule library
taaaggatagactcctcgac

SEQ ID NO: 208 - DNA, 20 bp, Artificial Sequence
  Sequence used in the synthesis of sub-barcode molecule library
taacggatagactcctcgac

SEQ ID NO: 209 - DNA, 20 bp, Artificial Sequence
  Sequence used in the synthesis of sub-barcode molecule library
taagggatagactcctcgac

SEQ ID NO: 210 - DNA, 20 bp, Artificial Sequence
  Sequence used in the synthesis of sub-barcode molecule library
taatggatagactcctcgac

SEQ ID NO: 211 - DNA, 20 bp, Artificial Sequence
  Sequence used in the synthesis of sub-barcode molecule library
tacaggatagactcctcgac

SEQ ID NO: 212 - DNA, 20 bp, Artificial Sequence
  Sequence used in the synthesis of sub-barcode molecule library
taccggatagactcctcgac

SEQ ID NO: 213 - DNA, 20 bp, Artificial Sequence
  Sequence used in the synthesis of sub-barcode molecule library
tacgggatagactcctcgac

SEQ ID NO: 214 - DNA, 20 bp, Artificial Sequence
  Sequence used in the synthesis of sub-barcode molecule library
tactggatagactcctcgac

SEQ ID NO: 215 - DNA, 20 bp, Artificial Sequence
  Sequence used in the synthesis of sub-barcode molecule library
tagaggatagactcctcgac

SEQ ID NO: 216 - DNA, 20 bp, Artificial Sequence
  Sequence used in the synthesis of sub-barcode molecule library
tagcggatagactcctcgac

SEQ ID NO: 217 - DNA, 20 bp, Artificial Sequence
  Sequence used in the synthesis of sub-barcode molecule library
taggggatagactcctcgac

SEQ ID NO: 218 - DNA, 20 bp, Artificial Sequence
  Sequence used in the synthesis of sub-barcode molecule library
tagtggatagactcctcgac

SEQ ID NO: 219 - DNA, 20 bp, Artificial Sequence
  Sequence used in the synthesis of sub-barcode molecule library
tataggatagactcctcgac

SEQ ID NO: 220 - DNA, 20 bp, Artificial Sequence
  Sequence used in the synthesis of sub-barcode molecule library
tatcggatagactcctcgac

SEQ ID NO: 221 - DNA, 20 bp, Artificial Sequence
  Sequence used in the synthesis of sub-barcode molecule library
tatgggatagactcctcgac

SEQ ID NO: 222 - DNA, 20 bp, Artificial Sequence
  Sequence used in the synthesis of sub-barcode molecule library
tattggatagactcctcgac

SEQ ID NO: 223 - DNA, 20 bp, Artificial Sequence
  Sequence used in the synthesis of sub-barcode molecule library
tcaaggatagactcctcgac

SEQ ID NO: 224 - DNA, 20 bp, Artificial Sequence
  Sequence used in the synthesis of sub-barcode molecule library
tcacggatagactcctcgac

SEQ ID NO: 225 - DNA, 20 bp, Artificial Sequence
  Sequence used in the synthesis of sub-barcode molecule library
tcagggatagactcctcgac

SEQ ID NO: 226 - DNA, 20 bp, Artificial Sequence
  Sequence used in the synthesis of sub-barcode molecule library
tcatggatagactcctcgac

SEQ ID NO: 227 - DNA, 20 bp, Artificial Sequence
  Sequence used in the synthesis of sub-barcode molecule library
tccaggatagactcctcgac

SEQ ID NO: 228 - DNA, 20 bp, Artificial Sequence
  Sequence used in the synthesis of sub-barcode molecule library
tcccggatagactcctcgac

SEQ ID NO: 229 - DNA, 20 bp, Artificial Sequence
  Sequence used in the synthesis of sub-barcode molecule library
tccgggatagactcctcgac

SEQ ID NO: 230 - DNA, 20 bp, Artificial Sequence
  Sequence used in the synthesis of sub-barcode molecule library
tcctggatagactcctcgac

SEQ ID NO: 231 - DNA, 20 bp, Artificial Sequence
  Sequence used in the synthesis of sub-barcode molecule library
tcgaggatagactcctcgac

SEQ ID NO: 232 - DNA, 20 bp, Artificial Sequence
  Sequence used in the synthesis of sub-barcode molecule library
tcgcggatagactcctcgac

SEQ ID NO: 233 - DNA, 20 bp, Artificial Sequence
  Sequence used in the synthesis of sub-barcode molecule library
tcggggatagactcctcgac

SEQ ID NO: 234 - DNA, 20 bp, Artificial Sequence
  Sequence used in the synthesis of sub-barcode molecule library
tcgtggatagactcctcgac

SEQ ID NO: 235 - DNA, 20 bp, Artificial Sequence
  Sequence used in the synthesis of sub-barcode molecule library
tctaggatagactcctcgac

SEQ ID NO: 236 - DNA, 20 bp, Artificial Sequence
  Sequence used in the synthesis of sub-barcode molecule library
tctcggatagactcctcgac

SEQ ID NO: 237 - DNA, 20 bp, Artificial Sequence
  Sequence used in the synthesis of sub-barcode molecule library
tctgggatagactcctcgac

SEQ ID NO: 238 - DNA, 20 bp, Artificial Sequence
  Sequence used in the synthesis of sub-barcode molecule library
tcttggatagactcctcgac

SEQ ID NO: 239 - DNA, 20 bp, Artificial Sequence
  Sequence used in the synthesis of sub-barcode molecule library
tgaaggatagactcctcgac

SEQ ID NO: 240 - DNA, 20 bp, Artificial Sequence
  Sequence used in the synthesis of sub-barcode molecule library
tgacggatagactcctcgac

SEQ ID NO: 241 - DNA, 20 bp, Artificial Sequence
  Sequence used in the synthesis of sub-barcode molecule library
tgagggatagactcctcgac

SEQ ID NO: 242 - DNA, 20 bp, Artificial Sequence
  Sequence used in the synthesis of sub-barcode molecule library
tgatggatagactcctcgac

SEQ ID NO: 243 - DNA, 20 bp, Artificial Sequence
  Sequence used in the synthesis of sub-barcode molecule library
tgcaggatagactcctcgac

SEQ ID NO: 244 - DNA, 20 bp, Artificial Sequence
  Sequence used in the synthesis of sub-barcode molecule library
tgccggatagactcctcgac

SEQ ID NO: 245 - DNA, 20 bp, Artificial Sequence
  Sequence used in the synthesis of sub-barcode molecule library
tgcgggatagactcctcgac

SEQ ID NO: 246 - DNA, 20 bp, Artificial Sequence
  Sequence used in the synthesis of sub-barcode molecule library
tgctggatagactcctcgac

SEQ ID NO: 247 - DNA, 20 bp, Artificial Sequence
  Sequence used in the synthesis of sub-barcode molecule library
tggaggatagactcctcgac

SEQ ID NO: 248 - DNA, 20 bp, Artificial Sequence
  Sequence used in the synthesis of sub-barcode molecule library
tggcggatagactcctcgac

SEQ ID NO: 249 - DNA, 20 bp, Artificial Sequence
  Sequence used in the synthesis of sub-barcode molecule library
tgggggatagactcctcgac

SEQ ID NO: 250 - DNA, 20 bp, Artificial Sequence
  Sequence used in the synthesis of sub-barcode molecule library
tggtggatagactcctcgac

SEQ ID NO: 251 - DNA, 20 bp, Artificial Sequence
  Sequence used in the synthesis of sub-barcode molecule library
tgtaggatagactcctcgac

SEQ ID NO: 252 - DNA, 20 bp, Artificial Sequence
  Sequence used in the synthesis of sub-barcode molecule library
tgtcggatagactcctcgac

SEQ ID NO: 253 - DNA, 20 bp, Artificial Sequence
  Sequence used in the synthesis of sub-barcode molecule library
tgtgggatagactcctcgac

SEQ ID NO: 254 - DNA, 20 bp, Artificial Sequence
  Sequence used in the synthesis of sub-barcode molecule library
tgttggatagactcctcgac

SEQ ID NO: 255 - DNA, 20 bp, Artificial Sequence
  Sequence used in the synthesis of sub-barcode molecule library
ttaaggatagactcctcgac

SEQ ID NO: 256 - DNA, 20 bp, Artificial Sequence
  Sequence used in the synthesis of sub-barcode molecule library
ttacggatagactcctcgac

SEQ ID NO: 257 - DNA, 20 bp, Artificial Sequence
  Sequence used in the synthesis of sub-barcode molecule library
ttagggatagactcctcgac

SEQ ID NO: 258 - DNA, 20 bp, Artificial Sequence
  Sequence used in the synthesis of sub-barcode molecule library
ttatggatagactcctcgac

SEQ ID NO: 259 - DNA, 20 bp, Artificial Sequence
  Sequence used in the synthesis of sub-barcode molecule library
ttcaggatagactcctcgac

SEQ ID NO: 260 - DNA, 20 bp, Artificial Sequence
  Sequence used in the synthesis of sub-barcode molecule library
ttccggatagactcctcgac

SEQ ID NO: 261 - DNA, 20 bp, Artificial Sequence
  Sequence used in the synthesis of sub-barcode molecule library
ttcgggatagactcctcgac

SEQ ID NO: 262 - DNA, 20 bp, Artificial Sequence
  Sequence used in the synthesis of sub-barcode molecule library
ttctggatagactcctcgac

SEQ ID NO: 263 - DNA, 20 bp, Artificial Sequence
  Sequence used in the synthesis of sub-barcode molecule library
ttgaggatagactcctcgac

SEQ ID NO: 264 - DNA, 20 bp, Artificial Sequence
  Sequence used in the synthesis of sub-barcode molecule library
ttgcggatagactcctcgac

SEQ ID NO: 265 - DNA, 20 bp, Artificial Sequence
  Sequence used in the synthesis of sub-barcode molecule library
ttggggatagactcctcgac

SEQ ID NO: 266 - DNA, 20 bp, Artificial Sequence
  Sequence used in the synthesis of sub-barcode molecule library
ttgtggatagactcctcgac

SEQ ID NO: 267 - DNA, 20 bp, Artificial Sequence
  Sequence used in the synthesis of sub-barcode molecule library
tttaggatagactcctcgac

SEQ ID NO: 268 - DNA, 20 bp, Artificial Sequence
  Sequence used in the synthesis of sub-barcode molecule library
tttcggatagactcctcgac

SEQ ID NO: 269 - DNA, 20 bp, Artificial Sequence
  Sequence used in the synthesis of sub-barcode molecule library
tttgggatagactcctcgac

SEQ ID NO: 270 - DNA, 47 bp, Artificial Sequence
  Primer
taatacgactcactatagggagataggacgatacgagtgtgtactcg

SEQ ID NO: 271 - DNA, 31 bp, Artificial Sequence
  Primer
ctgtcaaggtagactagcatgctcccatacg

SEQ ID NO: 272 - DNA, 24 bp, Artificial Sequence
  Primer
ctgtcaaggtagactagcatgctc

SEQ ID NO: 273 - DNA, 60 bp, Artificial Sequence
  Adaptor oligonucleotide
cctgactgctcgtcagttgattaagtactctgtgagatgatgatcagtagagcgagtcgt

SEQ ID NO: 274 - DNA, 50 bp, Artificial Sequence
  Primer
caccgagatctacactctttccctacacgacgctcttccgatcttgacct

SEQ ID NO: 275 - DNA, 59 bp, Artificial Sequence
  Synthetic template
  misc_feature (22)..(39): n is a, c, g, or t
atgatcagtagagcgagtcgtnnnnnnnnnnnnnnnnnntgctacgacttccgagtcca

SEQ ID NO: 276 - DNA, 27 bp, Artificial Sequence
  Primer
gctctactgatcattggactcggaagt

SEQ ID NO: 277 - DNA, 28 bp, Artificial Sequence
  Primer
tccgatcttggactcggaagtcgtagca

SEQ ID NO: 278 - DNA, 39 bp, Artificial Sequence; identical to SEQ ID NO: 17
  Primer

SEQ ID NO: 279 - DNA, 59 bp, Artificial Sequence
  BRCA1 adaptor oligonucleotide
cctgactgctcgtcagttgattaagtactctgtgagatgcttggcccctcttcggtaac

SEQ ID NO: 280 - DNA, 60 bp, Artificial Sequence
  BRCA1 adaptor oligonucleotide
cctgactgctcgtcagttgattaagtactctgtgagatgctgggagctcaaaagatggct

SEQ ID NO: 281 - DNA, 58 bp, Artificial Sequence
  BRCA1 adaptor oligonucleotide
cctgactgctcgtcagttgattaagtactctgtgagatgcagctgggctcaaaggacc

SEQ ID NO: 282 - DNA, 60 bp, Artificial Sequence
  BRCA1 adaptor oligonucleotide
cctgactgctcgtcagttgattaagtactctgtgagatgcagacaaattccccagcaggt

SEQ ID NO: 283 - DNA, 58 bp, Artificial Sequence
  BRCA1 adaptor oligonucleotide
cctgactgctcgtcagttgattaagtactctgtgagatgcatggtgtgaacccgggag

SEQ ID NO: 284 - DNA, 60 bp, Artificial Sequence
  BRCA1 adaptor oligonucleotide
cctgactgctcgtcagttgattaagtactctgtgagatgcccaaatcccaagtcgtgtgt

SEQ ID NO: 285 - DNA, 60 bp, Artificial Sequence
  BRCA1 adaptor oligonucleotide
cctgactgctcgtcagttgattaagtactctgtgagatgctcatccctggttccttgagg

SEQ ID NO: 286 - DNA, 60 bp, Artificial Sequence
  HLA-A adaptor oligonucleotide
cctgactgctcgtcagttgattaagtactctgtgaaaacggcctctgtggggagaagcaa

SEQ ID NO: 287 - DNA, 59 bp, Artificial Sequence
  HLA-A adaptor oligonucleotide
cctgactgctcgtcagttgattaagtactctgtgagatgcactgggctgaccgtggggt

SEQ ID NO: 288 - DNA, 60 bp, Artificial Sequence
  HLA-A adaptor oligonucleotide
cctgactgctcgtcagttgattaagtactctgcctgaatgwtctgactcttcccgtmaga

SEQ ID NO: 289 - DNA, 60 bp, Artificial Sequence
  DQB1 adaptor oligonucleotide
cctgactgctcgtcagttgattaagtactctgggatccccgcagaggatttcgtgtacca

SEQ ID NO: 290 - DNA, 60 bp, Artificial Sequence
  DQB1 adaptor oligonucleotide
cctgactgctcgtcagttgattaagtactctgtgagatggagcccacagtgaccatctcc

SEQ ID NO: 291 - DNA, 60 bp, Artificial Sequence
  BRCA1 reverse primer
cggtctcggcattcctgctgaaccgctcttccgatctgttctgagacacctgatgacctg

SEQ ID NO: 292 - DNA, 57 bp, Artificial Sequence
  BRCA1 reverse primer
cggtctcggcattcctgctgaaccgctcttccgatcttgaacccaggagtttgaggc

SEQ ID NO: 293 - DNA, 60 bp, Artificial Sequence
  BRCA1 reverse primer
cggtctcggcattcctgctgaaccgctcttccgatcttggagctttatctgctctgtgat

SEQ ID NO: 294 - DNA, 57 bp, Artificial Sequence
  BRCA1 reverse primer
cggtctcggcattcctgctgaaccgctcttccgatctttcactgtgatggccaggat

SEQ ID NO: 295 - DNA, 57 bp, Artificial Sequence
  BRCA1 reverse primer
cggtctcggcattcctgctgaaccgctcttccgatcttgacccaagcaagcctaaag

SEQ ID NO: 296 - DNA, 57 bp, Artificial Sequence
  BRCA1 reverse primer
cggtctcggcattcctgctgaaccgctcttccgatcttgccacagtagatgctcagt

SEQ ID NO: 297 - DNA, 58 bp, Artificial Sequence
  BRCA1 reverse primer
cggtctcggcattcctgctgaaccgctcttccgatctcccagcaaccatttcatttca

SEQ ID NO: 298 - DNA, 60 bp, Artificial Sequence
  HLA-A reverse primer
cggtctcggcattcctgctgaaccgctcttccgatcttggatctcggacccggagactgt

SEQ ID NO: 299 - DNA, 58 bp, Artificial Sequence
  HLA-A reverse primer
cggtctcggcattcctgctgaaccgctcttccgatctccctggtaccvgtgcgctgca

SEQ ID NO: 300 - DNA, 60 bp, Artificial Sequence
  HLA-A reverse primer
cggtctcggcattcctgctgaaccgctcttccgatctgaccctgctaaaggtctccagag

SEQ ID NO: 301 - DNA, 57 bp, Artificial Sequence
  HLA-A reverse primer
cggtctcggcattcctgctgaaccgctcttccgatctgaccctgctaaaggtcagag

SEQ ID NO: 302 - DNA, 60 bp, Artificial Sequence
  DQB1 reverse primer
cggtctcggcattcctgctgaaccgctcttccgatctaggacgctcacctctccgctgca

SEQ ID NO: 303 - DNA, 57 bp, Artificial Sequence
  DQB1 reverse primer
cggtctcggcattcctgctgaaccgctcttccgatctctggggtgctccacgtggca

SEQ ID NO: 304 - DNA, 20 bp, Artificial Sequence
  synthetic sequence
ggcccagcctacacacccat

SEQ ID NO: 305 - DNA, 20 bp, Artificial sequence
  synthetic sequence
ttacaagaattatacacctc

SEQ ID NO: 306 - DNA, 20 bp, Artificial Sequence
  synthetic sequence
ggcccaacctatacgcccat

SEQ ID NO: 307 - DNA, 20 bp, Artificial Sequence
  synthetic sequence
taacccttgtatttgaacac

SEQ ID NO: 308 - DNA, 20 bp, Artificial Sequence
  synthetic sequence
ctatccactgattaactgtg

SEQ ID NO: 309 - DNA, 20 bp, Artificial Sequence
  synthetic sequence
taatcgggccattaagcgat

SEQ ID NO: 310 - DNA, 20 bp, Artificial Sequence
  synthetic sequence
caagtaatttgctggaccta

SEQ ID NO: 311 - DNA, 16 bp, Artificial Sequence
  synthetic sequence
taccagagatacttgc

SEQ ID NO: 312 - DNA, 14 bp, Artificial Sequence
  synthetic sequence
atatccagcaaagt

SEQ ID NO: 313 - DNA, 13 bp, Artificial Sequence
  synthetic sequence
ttacaagaattat

SEQ ID NO: 314 - DNA, 82 bp, Artificial Sequence
  synthetic sequence
atacctgactgctcgtcagttgagcgaattccgtatggtggtacacacctacactactcggacgctcttccgatcttgacct

SEQ ID NO: 315 - DNA, 25 bp, Artificial Sequence
  synthetic sequence
gctcaactgacgagcagtcaggtat

SEQ ID NO: 316 - DNA, 82 bp, Artificial Sequence
  synthetic sequence
aggtcaagatcggaagagcgtccgagtagtgtaggtgtgtaccaccatacggaattcgctcaactgacgagcagtcaggtat

SEQ ID NO: 317 - DNA, 28 bp, Artificial Sequence
  synthetic sequence
  misc_feature (22)..(28): n is a, c, g, or t
tacacgacgctcttccgatctnnnnnnn

SEQ ID NO: 318 - DNA, 18 bp, Artificial Sequence
  synthetic sequence
acgacgctcttccgatct

SEQ ID NO: 319 - DNA, 20 bp, Artificial Sequence
  synthetic sequence
gtgactggagttcagacgtg

SEQ ID NO: 320 - DNA, 80 bp, Artificial Sequence
  synthetic sequence
  misc_feature (35)..(50): n is a, c, g, or t
gtgactggagttcagacgtgtgctcttccgatctnnnnnnnnnnnnnnnncgtgcttttttttttttttttttttttttt

SEQ ID NO: 321 - DNA, 35 bp, Artificial Sequence
  synthetic sequence
  modified_base (35)..(35): 3′ inverted dT
  misc_feature (35)..(35): n is a, c, g, or t
agatcggaagagcacacgtctgaactccagtcacn

SEQ ID NO: 322 - DNA, 18 bp, Artificial Sequence
  synthetic sequence
gtgactggagttcagacg

SEQ ID NO: 323 - DNA, 30 bp, Artificial Sequence
  synthetic sequence
aaaaaaaaaaaaaaaaaaaaaaaaagcacg

SEQ ID NO: 324 - DNA, 18 bp, Artificial Sequence
  synthetic sequence
gtgactggagttcagacg

SEQ ID NO: 325 - DNA, 30 bp, Artificial Sequence; identical to SEQ ID NO: 323
  synthetic sequence

SEQ ID NO: 326 - DNA, 90 bp, Artificial Sequence
  synthetic sequence
  misc_feature (34)..(59): n is a, c, g, or t
  modified_base (90)..(90): 3′ Inverted dT
  misc_feature (90)..(90): n is a, c, g, or t
acactctttccctacacgacgctcttccgatctnnnnnnnnnnnnnnnnnnnnnnnnnnaaaaaaaaaaaaaaaaaaaaaaaaagcacgn

SEQ ID NO: 327 - DNA, 22 bp, Artificial Sequence
  synthetic sequence
cgtgaccagcgactcagccttt

SEQ ID NO: 328 - DNA, 21 bp, Artificial Sequence
  synthetic sequence
tttagatcggaagagcacttt

SEQ ID NO: 329 - DNA, 18 bp, Artificial Sequence
  synthetic sequence
tttagatcggaagagcac

SEQ ID NO: 330 - DNA, 21 bp, Artificial Sequence
  synthetic sequence
cgtgaccagcgactcagcctt

SEQ ID NO: 331 - DNA, 17 bp, Artificial Sequence
  synthetic sequence
ttagatcggaagagcac

SEQ ID NO: 332 - DNA, 20 bp, Artificial Sequence
  synthetic sequence
tttgatcggaagagcacttt

SEQ ID NO: 333 - DNA, 17 bp, Artificial Sequence
  synthetic sequence
tttgatcggaagagcac

SEQ ID NO: 334 - DNA, 20 bp, Artificial Sequence
  synthetic sequence
tttagatcggaagagcattt

SEQ ID NO: 335 - DNA, 17 bp, Artificial Sequence
  synthetic sequence
tttagatcggaagagca

SEQ ID NO: 336 - DNA, 19 bp, Artificial Sequence
  synthetic sequence
tttgatcggaagagcattt

SEQ ID NO: 337 - DNA, 16 bp, Artificial Sequence
  synthetic sequence
tttgatcggaagagca

SEQ ID NO: 338 - DNA, 27 bp, Artificial Sequence
  synthetic sequence
tttagatcggaagagcacacgtctttt

SEQ ID NO: 339 - DNA, 24 bp, Artificial Sequence
  synthetic sequence
tttagatcggaagagcacacgtct

SEQ ID NO: 340 - DNA, 21 bp, Artificial Sequence
  synthetic sequence
ttttttttttttttttttttt

SEQ ID NO: 341 - DNA, 88 bp, Artificial Sequence
  synthetic sequence
cgtgaccagcgactcagccttttttagatcggaagagcacttttttagatcggaagagcacttttttagatcggaagagcactttttt

SEQ ID NO: 342 - DNA, 19 bp, Artificial Sequence
  synthetic sequence
ggctgagtcgctggtcacg

SEQ ID NO: 343 - DNA, 80 bp, Artificial Sequence
  synthetic sequence
gtgactggagttcagacgtgtgctcttccgatctgtagtgattccaattacgtgcttttttttttttttttttttttttt

SEQ ID NO: 344 - DNA, 60 bp, Artificial Sequence
  synthetic sequence
ggagttcagacgtgtgctcttccgatcttttttaattttttttttttttttttttttttt

SEQ ID NO: 345 - DNA, 12 bp, Artificial Sequence
  synthetic sequence
  modified_base (12)..(12): 3′ inverted dT
  misc_feature (12)..(12): n is a, c, g, or t
atcggaagagcn

SEQ ID NO: 346 - DNA, 16 bp, Artificial Sequence
  synthetic sequence
  modified_base (16)..(16): 3′ inverted dT
  misc_feature (16)..(16): n is a, c, g, or t
agatcggaagagcacn

SEQ ID NO: 347 - DNA, 26 bp, Artificial Sequence
  synthetic sequence
  modified_base (26)..(26): 3′ inverted dT
  misc_feature (26)..(26): n is a, c, g, or t
agatcggaagagcacacgtctgaacn

SEQ ID NO: 348 - DNA, 16 bp, Artificial Sequence
  synthetic sequence
gtgcagttgggacgct

SEQ ID NO: 349 - DNA, 100 bp, Artificial Sequence
  synthetic sequence
  modified_base (100)..(100): 3′ inverted dT
  misc_feature (100)..(100): n is a, c, g, or t
gtgcagttgggacgctgaagtaacgcaacttatgaggaaggctaatatagcaggaactaaattccagtgaaaaaaaaaaaaaaaaaaaaaaaaaagatcn

SEQ ID NO: 350 - DNA, 20 bp, Artificial Sequence
  synthetic sequence
gtgactggagttcagacgtg

SEQ ID NO: 351 - DNA, 80 bp, Artificial Sequence; identical to SEQ ID NO: 320
  synthetic sequence
  misc_feature (35)..(50): n is a, c, g, or t

SEQ ID NO: 352 - DNA, 60 bp, Artificial Sequence; identical to SEQ ID NO: 344
  synthetic sequence

SEQ ID NO: 353 - DNA, 96 bp, Artificial Sequence
  synthetic sequence
  modified_base (96)..(96): 3′ inverted dT
  misc_feature (96)..(96): n is a, c, g, or t
gtgcagttgggacgctgaagtaacgcaacttatgaggaaggctaatatagcaggaactaaattccagtgaaaaaaaaaaaaaaaaaaaaaaaaatn

SEQ ID NO: 354 - DNA, 16 bp, Artificial Sequence
  synthetic sequence
gtgcagttgggacgct

SEQ ID NO: 355 - DNA, 19 bp, Artificial Sequence
  synthetic sequence
ggagttcagacgtgtgctc

SEQ ID NO: 356 - DNA, 27 bp, Artificial Sequence
  synthetic sequence
  modified_base (27)..(27): 3′ inverted dT
  misc_feature (27)..(27): n is a, c, g, or t
aaaaaaaaaaaaaaaaaaaaaaaaaan

SEQ ID NO: 357 - DNA, 16 bp, Artificial Sequence
  synthetic sequence
  modified_base (16)..(16): 3′ inverted dT
  misc_feature (16)..(16): n is a, c, g, or t
gtgctcttccgatctn

(19) United States
(12) Patent Application Publication
(10) Pub. No.: US 2026/0078365 A1
(43) Pub. Date: Mar. 19, 2026

(54) Reagents and Methods for Molecular Barcoding

(71) Applicant: CS GENETICS LIMITED, Cambridge, Cambridgeshire (GB)

(72) Inventor: Lucas Brandon Edelman, Cambridge, Cambridgeshire (GB)

(21) Appl. No.: 18/568,695

(22) PCT Filed: Jun. 17, 2022
(86) PCT No.: PCT/GB2022/051541

§ 371 Date: Dec. 8, 2023

(30) Foreign Application Priority Data
Jun. 18, 2021 (GB) 2108841.4
Dec. 17, 2021 (GB) 2118477.5

(51) Int. Cl. C12N 15/10

(52) U.S. Cl. CPC C12N 15/1065; C12N 15/1096

(57) ABSTRACT

Reagents and methods for preparing nucleic acid samples for sequencing are provided. The reagents include multimeric barcoding reagents that comprise barcode regions linked together and a cell-binding moiety. The methods comprise contacting a nucleic acid sample comprising cells with a library of multimeric barcoding reagents, wherein each multimeric barcoding reagent comprises barcode regions linked together, and appending barcode sequences of a first multimeric barcoding reagent to sub-sequences of a target nucleic acid of a first cell, and appending barcode sequences of a second multimeric barcoding reagent to sub-sequences of a target nucleic acid of a second cell. Methods are also provided that comprise steps of internalising multimeric barcoding reagents into cells (e.g. by endocytosis) or exposing multimeric barcoding reagents to target nucleic acids by lysing cells or permeabilizing cell membranes.

### TECHNICAL FIELD

The present invention relates to molecular barcoding. Provided are libraries of multimeric barcoding reagents and methods for their use in barcoding nucleic acids of single cells and microparticles.

### INCORPORATION BY REFERENCE OF SEQUENCE LISTING PROVIDED AS A TEXT FILE

A Sequence Listing is provided herewith as a text file, “BOULT-054_SEQLIST_7-18-25_ST25.txt” created on Jul. 18, 2025 and having a size of 82,537 bytes. The contents of the text file are incorporated by reference herein in their entirety.

### BACKGROUND

‘Molecular barcoding’ was developed to address problems generated by raw error rates intrinsic to DNA sequence machines (synthetic accuracy), and also problems related to counting individual nucleic acid molecules within a sample (molecular counting).

Molecular barcoding generally involves attaching (for example, by ligation or by primer-extension) a unique nucleic acid label (a ‘barcode’) to several single target molecules (DNA or RNA) in a solution containing a large number of such molecules. These labelled molecules are then sequenced, which for each reveals both the sequence of the molecular barcode, and at least part of the sequence of the labelled target molecule itself.

This barcoding is typically used towards two different ends. First, it can be used to enable ‘redundant sequencing’. For example, imagine a nucleic acid sample containing 1000 copies of a particular gene in a DNA sample; 999 of the copies hold sequences identical to each other, but a single copy has a particular single-nucleotide mutation. Without barcoding, the sequencer will be unable to detect this mutated copy, since the sequencer makes random errors at a higher rate than 1:1000—i.e. the mutation is so rare in the population of sequenced molecules that it falls below the sequencer's intrinsic background noise threshold.

However, if the 1000 copies have each been labelled with a unique molecular barcode, and each individual labelled molecule is sequenced several times by the sequencing machine (redundant sequencing), you would observe that every time (or, at least 99% of the time, equivalent to the raw accuracy of the sequencer) that the labelled mutated molecule was redundantly sequenced (i.e, every time the target gene sequence was observed to be labelled with that one particular unique barcode that was attached to the mutated starting molecule), that the same apparent mutation would in fact be observed. By contrast, that particular mutation would only be observed approximately 1% of the time (the raw error rate of the sequencer) when the labelled but non-mutated gene copies were redundantly sequenced, as per their respective alternative barcodes.

The barcode thus serves to identify individual input molecules across all their respective multiple copies within the sequencing reaction, allowing a sequence-detection algorithm to specifically focus on their respective reads within a sequencing dataset, and thus avoiding the large amount of stochastic sequence noise (in the form of sequence errors) that is present across the remainder of the dataset. This thus enables ‘synthetic accuracy’, through redundant sequencing, which is potentially much higher than the raw accuracy of the sequencer itself.

Barcoding can also be used to enable digital ‘molecular counting’ of input DNA or RNA molecules. In this process, a large number of unique barcodes are attached to input molecules, for example, cDNA copies that have been made from a particular mRNA species. Each input cDNA molecule is labelled (for example, by primer extension) with a single, unique barcode. The molecules are then sequenced, which, as with redundant sequencing, reveals the unique barcode and at least part of each associated labelled input molecule; these molecules are then also each sequenced more than once.

Instead of using this redundant sequencing to reduce sequencing errors, in molecular counting it is used to digitally quantify how many individual molecules of the given target molecule (cDNA in this case) were present in the original sample, by simply counting the total number of unique barcodes that were sequenced and found to be associated with the particular target. Barcode-directed redundant sequencing in this way reduces the chance that any input molecule is stochastically left unsequenced by the sequencing reaction (since each labelled molecule on average is sequenced several times), whilst retaining an accurate measure of input quantity (since redundantly sequenced starting molecules are only counted once, as discriminated by repeated copies of their unique barcode).

Examples of the use of molecular barcodes are provided in U.S. Pat. Nos. 8,728,766, 8,685,678, 8,722,368, Kinde et al., 2011 (PNAS, 108, 23, 9530-9535) and US20140227705 A1.

A ‘synthetic long read’ is generated when a long, contiguous sequence of DNA (longer than the readlength attainable on a DNA sequencer) is converted into two or more shorter ‘sub-sequences’ that are short enough to be read by a DNA sequencer, and which are somehow labelled such that it can be deduced (after sequencing) that the sub-sequences were generated from the same original long DNA sequence. For example, if you want to sequence a particular human gene which is 1000 nucleotides long, but do so with a short-read DNA sequencer with a readlength of 100 nucleotides, you could separate the long sequence into 10 different sub-sequences of 100 nucleotide length, then label each of these 10 sub-sequences with a synthetic, informative ‘label’DNA sequence that identifies each of the 10 sub-sequences as coming from the same original 1000 nucleotide DNA molecule, then perform high-throughput DNA sequencing with these 10 resulting DNA molecules, and thus (for each of the 10 resulting DNA molecules) attain both the 100 nucleotide sub-sequence, and the associated identifying DNA label. With this high-throughput DNA data an algorithm can be used which detects these identifying labels and uses them to associate the 10 different 100-nucleotide subsequences with each other as a collective sub-sequence ‘grouping’, and therewith estimate that the 10 sub-sequences came from a longer, 1000-nucleotide gene, and therewith estimate the total 1000-nucleotide long genetic sequence by ‘stitching’ the 10 sub-sequences together in silico into a single 1000-nucleotide long gene.

At least two general synthetic long read technologies have been described in the literature: a partitioning-based approach which is described in US20130079231 A1 and US2014378345 A1; and a barcode-copying approach which is described in Casbon et al., 2013 (Nucleic Acids Research, 2013, 41, 10, e112), U.S. Pat. Nos. 8,679,756 and 8,563,274.

‘Spatial sequencing’ is considered to be the sequencing of nucleic acids with the inclusion of some information about where each sequenced nucleic acid is located within a particular space (for example, within a particular sample, or within a particular cell). However, very few spatial sequencing methods are known. The main known technology is the fluorescent in situ RNA sequencing (FISSEQ) technique. In FISSEQ a sample of cells are cross-linked, and while the cells are still intact, RNA is reverse transcribed into cDNA, and amplified whilst still in the crosslinked cells. Then, each amplified cDNA molecule is sequenced optically whilst still in the cells, with a high-powered and sensitive optical detection system. This method is described in Lee et al., 2014 (Science, 343, 6177, 1360-1363). Current techniques for performing nucleic acid analysis of single cells are generally limited in throughput (ie, the number of cells that may be simultaneously analysed within a single experiment, or analysed per unit time), and also require relatively complex experimental instrumentation, such as microfluidic equipment, and may furthermore involve relatively complex and/or length experimental procedures to carry out.

The invention addresses two main types of problem in the sequencing field: 1) specific analytic limitations of DNA sequencing machines; and 2) biophysical challenges associated with common types of experimental DNA samples.

Current high-throughput DNA-sequencing machines are powerful platforms used to analyse large amounts of genetic material (from thousands to billions of DNA molecules) and function as systems for both basic research and applied medical applications. However, all current DNA sequencing machines are subject to certain analytic limitations which constrain the scientific and medical applications in which they can be effectively used. The chief such limitations include finite raw readlengths and finite raw accuracy, both of which are described below.

With regard to finite raw readlengths, each DNA sequencing platform is characterised by a typical ‘readlength’ that it can attain, which is the ‘length’ in nucleotides of DNA that it can ‘read’ of each sequenced molecule. For most sequencing machines, this ranges from 100 to ˜500 nucleotides.

With regard to finite raw accuracy, each sequencing platform is also characterised by an attainable ‘raw accuracy’, typically defined as the likelihood that each given nucleotide it sequences has been determined correctly. Typical raw accuracy for the most popular sequencing platforms range between 98 and 99.5%. The related quantity, the ‘raw error’ rate, is essentially the converse of raw accuracy, and is the per-nucleotide likelihood that the sequencer randomly reports an incorrect nucleotide in a particular sequenced DNA molecule.

In addition, certain common experimental DNA samples pose biophysical challenges for sequencing. These challenges arise from the unique (and troublesome) molecular state of DNA in these samples, which makes it difficult to sequence them or to extract important pieces of genetic information therefrom, irrespective of the sequencing machine employed. For example, Formalin-Fixed Paraffin-Embedded (FFPE) samples are the standard experimental tool for performing molecular pathology from human biopsy specimens. However, the process of creating an FFPE sample—in which the biopsy specimen is fixed (crosslinked and kept physically together and stable at the molecular level) by a harsh chemical, and then embedded in a wax-creates significant damage to the DNA and RNA contained therein. DNA and RNA from FFPE samples is thus heavily fragmented (generally into small fragments between 50 and 200 nucleotides), and also includes sporadic damage to individual nucleotides which makes it essentially impossible to amplify or isolate long, contiguous sequences.

### DESCRIPTION

The invention provides multimeric barcoding reagents and methods for their use in preparing nucleic acid samples containing cells and/or microparticles for sequencing. In the methods, the multimeric barcoding reagents are used to barcode target nucleic acids of cells and/or microparticles in the samples. Barcode sequences may be appended from a single multimeric barcoding reagent to sub-sequences of a target nucleic acid of a single cell (or single microparticle) to produce a set of barcoded target nucleic acid molecules. Such molecules may be sequenced to produce sets of sequence reads, each set of sequence reads corresponding to nucleic acid molecules of a single cell (i.e. single-cell sequencing) or a single microparticle. In addition, the methods may be performed on many cells (or many microparticles) in parallel enabling high throughput single-cell sequencing and/or high throughput single-microparticle sequencing.

The applicant has previously provided reagents and methods related to barcoding. In WO2016/207639, the applicant provided a wide range of reagents, kits and methods for molecular barcoding including multimeric barcoding reagents. In WO/2018/115849, the applicant provided further methods and reagents for molecular barcoding.

In WO2018/115855, the applicant provided methods for the analysis of nucleic acid fragments in microparticles (e.g. circulating microparticles, or microparticles originating from blood). That invention is based on a linked-fragment approach in which fragments of nucleic acid from a single microparticle are linked together. This linkage enables the production of a set of linked sequence reads (i.e. set of linked signals) corresponding to the sequences of fragments from a single microparticle.

In WO/2018/115852, the applicant provided reagents and methods for molecular barcoding of nucleic acids of single cells.

In WO2020/002862 and WO2020/115511, the applicant provided further reagents and methods for the analysis of biomolecules (e.g. nucleic acids and polypeptides) of cell-free microparticles or cells.

The present invention provides further reagents, libraries and methods for molecular barcoding of nucleic acids of single cells and single microparticles.

The entire content of WO2016/207639, WO/2018/115849, WO/2018/115852, WO/2018/115855, WO2020/002862 and WO2020/115511 is incorporated herein by reference.

The invention provides a method of preparing a nucleic acid sample for sequencing, wherein the sample comprises at least 2 cells, and wherein the method comprises in order the steps of:
(a) contacting the sample with a library comprising at least two multimeric barcoding reagents, wherein each multimeric barcoding reagent comprises first and second barcoded oligonucleotides linked together, wherein the barcoded oligonucleotides each comprise a barcode region and wherein the barcode regions of the first and second barcoded oligonucleotides of a first multimeric barcoding reagent of the library are different to the barcode regions of the first and second barcoded oligonucleotides of a second multimeric barcoding reagent of the library; and
(b) freezing the cells.

The invention provides a frozen sample obtainable by any of the methods described herein.

A method of preparing a nucleic acid sample for sequencing, wherein the sample comprises at least 2 cells, and wherein the method comprises in order the steps of:
(a) contacting the sample with a library comprising at least two multimeric barcoding reagents, wherein each multimeric barcoding reagent comprises first and second barcoded oligonucleotides linked together, wherein the barcoded oligonucleotides each comprise a barcode region and wherein the barcode regions of the first and second barcoded oligonucleotides of a first multimeric barcoding reagent of the library are different to the barcode regions of the first and second barcoded oligonucleotides of a second multimeric barcoding reagent of the library;
(b) lysing the cells or permeabilizing the cell membranes of the cells; and
(c) appending (e.g. annealing or ligating) the first and second barcoded oligonucleotides of the first multimeric barcoding reagent to first and second sub-sequences of a target nucleic acid of the first cell to produce first and second barcoded target nucleic acid molecules, and appending the first and second barcoded oligonucleotides from the second multimeric barcoding reagent to first and second sub-sequences of a target nucleic acid of the second cell to produce first and second barcoded target nucleic acid molecules;
wherein the method further comprises (i) freezing the cells and, optionally, (ii) thawing the cells, optionally wherein the cells are comprised within a single contiguous aqueous volumne during steps (a), (b) and/or (c). The cells may be comprised within a single contiguous aqueous volume during steps (a) and (b), steps (b) and (c), or steps (a), (b) and (c).

The invention provides a method of preparing a nucleic acid sample for sequencing, wherein the sample comprises at least 2 cells, and wherein the method comprises in order the steps of:
(a) contacting the sample with a library comprising at least two multimeric barcoding reagents, wherein each multimeric barcoding reagent comprises first and second barcoded oligonucleotides linked together, wherein the barcoded oligonucleotides each comprise a barcode region and wherein the barcode regions of the first and second barcoded oligonucleotides of a first multimeric barcoding reagent of the library are different to the barcode regions of the first and second barcoded oligonucleotides of a second multimeric barcoding reagent of the library, wherein the first multimeric barcoding reagent binds to the cell membrane of a first cell prior to step (b), and wherein the second multimeric barcoding reagent binds to the cell membrane of a second cell prior to step (b);
(b) lysing the cells or permeabilizing the cell membranes of the cells; and
(c) appending (e.g. annealing or ligating) the first and second barcoded oligonucleotides of the first multimeric barcoding reagent to first and second sub-sequences of a target nucleic acid of the first cell to produce first and second barcoded target nucleic acid molecules, and appending (e.g. annealing or ligating) the first and second barcoded oligonucleotides from the second multimeric barcoding reagent to first and second sub-sequences of a target nucleic acid of the second cell to produce first and second barcoded target nucleic acid molecules;
wherein the method further comprises (i) freezing the cells and, optionally, (ii) thawing the cells, optionally wherein the cells are comprised within a single contiguous aqueous volumne during steps (a), (b) and/or (c). The cells may be comprised within a single contiguous aqueous volume during steps (a) and (b), steps (b) and (c), or steps (a), (b) and (c).

The invention provides a method of preparing a nucleic acid sample for sequencing, wherein the sample comprises at least 2 cells, and wherein the method comprises in order the steps of:
(a) contacting the sample with a library comprising at least two multimeric barcoding reagents, wherein each multimeric barcoding reagent comprises first and second barcoded oligonucleotides linked together and a cell-binding moiety, wherein the barcoded oligonucleotides each comprise a barcode region and wherein the barcode regions of the first and second barcoded oligonucleotides of a first multimeric barcoding reagent of the library are different to the barcode regions of the first and second barcoded oligonucleotides of a second multimeric barcoding reagent of the library, wherein the cell-binding moiety of the first multimeric barcoding reagent binds to the cell membrane of a first cell prior to step (b), and wherein the cell-binding moiety of the second multimeric barcoding reagent binds to the cell membrane of a second cell prior to step (b);
(b) lysing the cells or permeabilizing the cell membranes of the cells; and
(c) appending (e.g. annealing or ligating) the first and second barcoded oligonucleotides of the first multimeric barcoding reagent to first and second sub-sequences of a target nucleic acid of the first cell to produce first and second barcoded target nucleic acid molecules, and appending (e.g. annealing or ligating) the first and second barcoded oligonucleotides from the second multimeric barcoding reagent to first and second sub-sequences of a target nucleic acid of the second cell to produce first and second barcoded target nucleic acid molecules;
wherein the method further comprises (i) freezing the cells and, optionally, (ii) thawing the cells, optionally wherein the cells are comprised within a single contiguous aqueous volumne during steps (a), (b) and/or (c). The cells may be comprised within a single contiguous aqueous volume during steps (a) and (b), steps (b) and (c), or steps (a), (b) and (c).

The invention provides a method of preparing a nucleic acid sample for sequencing, wherein the sample comprises at least 2 cells, and wherein the method comprises in order the steps of:
(a) contacting the sample with a library comprising at least two multimeric barcoding reagents, wherein each multimeric barcoding reagent comprises
   (i) a support,
   (ii) at least two multimeric hybridization molecules, wherein each multimeric hybridization molecule is independently linked to the support and wherein each multimeric hybridization molecule comprises at least two hybridization molecules linked together, wherein each of the hybridization molecules comprises a nucleic acid sequence comprising a hybridization region, and
   (iii) at least two barcoded oligonucleotides annealed to each of the multimeric hybridization molecules, wherein each barcoded oligonucleotide is annealed to one of the hybridization regions and wherein each barcoded oligonucleotide comprises a barcode region,
   and wherein the barcode regions of the barcoded oligonucleotides of a first multimeric barcoding reagent of the library are different to the barcode regions of the barcoded oligonucleotides of a second multimeric barcoding reagent of the library;
(b) lysing the cells or permeabilizing the cell membranes of the cells; and
(c) (separately) appending (e.g. annealing or ligating) each of the barcoded oligonucleotides of the first multimeric barcoding reagent to at least four sub-sequences of a target nucleic acid of the first cell to produce at least four barcoded target nucleic acid molecules, and (separately) appending (e.g. annealing or ligating) each of the barcoded oligonucleotides of the second multimeric barcoding reagent to a least four sub-sequences of a target nucleic acid of the second cell to produce at least four barcoded target nucleic acid molecules;
wherein the method further comprises (i) freezing the cells and, optionally, (ii) thawing the cells, optionally wherein the cells are comprised within a single contiguous aqueous volumne during steps (a), (b) and/or (c). The cells may be comprised within a single contiguous aqueous volume during steps (a) and (b), steps (b) and (c), or steps (a), (b) and (c).

The invention provides a method of preparing a nucleic acid sample for sequencing, wherein the sample comprises at least 2 cells, and wherein the method comprises in order the steps of:
(a) contacting the sample with a library comprising at least two multimeric barcoding reagents, wherein each multimeric barcoding reagent comprises
   (i) a support,
   (ii) at least two multimeric hybridization molecules, wherein each multimeric hybridization molecule is independently linked to the support and wherein each multimeric hybridization molecule comprises at least two hybridization molecules linked together, wherein each of the hybridization molecules comprises a nucleic acid sequence comprising a hybridization region, and
   (iii) at least two barcoded oligonucleotides annealed to each of the multimeric hybridization molecules, wherein each barcoded oligonucleotide is annealed to one of the hybridization regions and wherein each barcoded oligonucleotide comprises a barcode region, and wherein the barcode regions of the barcoded oligonucleotides of a first multimeric barcoding reagent of the library are different to the barcode regions of the barcoded oligonucleotides of a second multimeric barcoding reagent of the library, wherein the first multimeric barcoding reagent binds to the cell membrane of a first cell prior to step (b), and wherein the second multimeric barcoding reagent binds to the cell membrane of a second cell prior to step (b);
(b) lysing the cells or permeabilizing the cell membranes of the cells; and
(c) (separately) appending (e.g. annealing or ligating) each of the barcoded oligonucleotides of the first multimeric barcoding reagent to at least four sub-sequences of a target nucleic acid of the first cell to produce at least four barcoded target nucleic acid molecules, and (separately) appending (e.g. annealing or ligating) each of the barcoded oligonucleotides of the second multimeric barcoding reagent to a least four sub-sequences of a target nucleic acid of the second cell to produce at least four barcoded target nucleic acid molecules;
wherein the method further comprises (i) freezing the cells and, optionally, (ii) thawing the cells, optionally wherein the cells are comprised within a single contiguous aqueous volumne during steps (a), (b) and/or (c). The cells may be comprised within a single contiguous aqueous volume during steps (a) and (b), steps (b) and (c), or steps (a), (b) and (c).

The invention provides a method of preparing a nucleic acid sample for sequencing, wherein the sample comprises at least 2 cells, and wherein the method comprises in order the steps of:
(a) contacting the sample with a library comprising at least two multimeric barcoding reagents, wherein each multimeric barcoding reagent comprises
   (i) a support,
   (ii) at least two multimeric hybridization molecules, wherein each multimeric hybridization molecule is independently linked to the support and wherein each multimeric hybridization molecule comprises at least two hybridization molecules linked together, wherein each of the hybridization molecules comprises a nucleic acid sequence comprising a hybridization region,
   (iii) at least two barcoded oligonucleotides annealed to each of the multimeric hybridization molecules, wherein each barcoded oligonucleotide is annealed to one of the hybridization regions and wherein each barcoded oligonucleotide comprises a barcode region, and
   (iv) a cell-binding moiety linked to each multimeric hybridization molecule,
   and wherein the barcode regions of the barcoded oligonucleotides of a first multimeric barcoding reagent of the library are different to the barcode regions of the barcoded oligonucleotides of a second multimeric barcoding reagent of the library, wherein the cell-binding moiety of the first multimeric barcoding reagent binds to the cell membrane of a first cell prior to step (b), and wherein the cell-binding moiety of the second multimeric barcoding reagent binds to the cell membrane of a second cell prior to step (b);
(b) lysing the cells or permeabilizing the cell membranes of the cells; and
(c) (separately) appending (e.g. annealing or ligating) each of the barcoded oligonucleotides of the first multimeric barcoding reagent to at least four sub-sequences of a target nucleic acid of the first cell to produce at least four barcoded target nucleic acid molecules, and (separately) appending (e.g. annealing or ligating) each of the barcoded oligonucleotides of the second multimeric barcoding reagent to a least four sub-sequences of a target nucleic acid of the second cell to produce at least four barcoded target nucleic acid molecules;
wherein the method further comprises (i) freezing the cells and, optionally, (ii) thawing the cells, optionally wherein the cells are comprised within a single contiguous aqueous volumne during steps (a), (b) and/or (c). The cells may be comprised within a single contiguous aqueous volume during steps (a) and (b), steps (b) and (c), or steps (a), (b) and (c).

The incorporation of step(s) involving the freezing of cells and/or the frozen storage of cells for a duration of time (for example, freezing cells following a step of contacting them with a library of multimeric barcoding reagents, and/or freezing a sample of cells following a step of binding a library of multimeric barcoding reagents to said sample of cells, and/or frozen storage of cells following a step of contacting them with a library of multimeric barcoding reagents, and/or frozen storage of a sample of cells following a step of binding a library of multimeric barcoding reagents to said sample of cells) in the method provides operational flexibility as it allows a pause in the workflow. This provides a range of advantages. For example, it allows cell and sample types with varied sample preparation times (and/or varied availability and/or accessibility of the individual cell samples themselves) to be prepared and analysed in parallel. In addition, it allows users to bank samples for long-term storage; this is currently not feasible with single cell analysis technologies. Surprisingly, the inventors have found that samples can be stored frozen for long periods without affecting the ability to successfully perform step (c) and without adversely affecting the sequence data obtained from the samples. Moreover, the inventors have unexpectedly demonstrated that performing steps (b) (i) (freezing the cells) and (b) (ii) (thawing the cells) can enhance lysis/permeabiliziation of the cells exposing the barcoded oligonucleotides to the target nucleic acids of the cells and, therefore, improves the efficiency of step (c).

In the methods, the step of (i) freezing the cells, and optionally the step of (ii) thawing the cells, may be performed before, during or after any of the other steps. The step of (i) freezing the cells, and optionally the step of (ii) thawing the cells, may be performed after step (a) and, optionally, prior to step (c). For example, the step of (i) freezing the cells (and optionally the step of (ii) thawing the cells) may be performed between the step (a) of contacting the sample with a library comprising at least two multimeric barcoding reagents, and the step (c) of appending (e.g. annealing or ligating) barcoded oligonucleotides.

The step of (i) freezing the cells, and optionally the step of (ii) thawing the cells, may be performed as part of step (b).

In the methods, step (b) (the step of lysing the cells or permeabilizing the cell membranes of the cells) may comprise (i) freezing the cells, and, optionally, (ii) thawing the cells.

The methods may be methods of preparing a nucleic acid sample for single cell whole transcriptome sequencing.

In the methods, step (a) (the step of contacting the sample with a library at least two multimeric barcoding reagents) may comprise: (i) forming a layer comprising the library of at least two multimeric barcoding reagents; and (ii) contacting the layer with the sample.

By performing steps (a) (i) then (a) (ii), the period during which the sample is manipulated prior to cell lysis may be minimised which increases the likelihood that the cells present in the sample are still viable at the point of cell lysis.

In step (a) (i), the layer comprising the library of multimeric barcoding reagents may be formed by gravity and/or centrifugation. The layer may be formed in a reaction vessel e.g. a tube or a well on a plate. The centrifugation may comprise a single step of centrifugation or a series of steps of centrifugation. A step of centrigugation may be performed at at least 50 G, at least 100 G, at least 200 G, at least 300 G, at least 500 G, at least 750 G, at least 1000 G, at least 1200 G, at least 1500 G, or at least 2000 G. A step of centrifugation may be performed for a duration of at least 5 seconds, at least 10 seconds, at least 30 seconds, at least 60 seconds, at least 5 minutes, at least 10 minutes or at least 30 minutes.

In step (a) (ii), the layer comprising the library of multimeric barcoding reagents may be contacted with the sample by allowing the sample to settle on the multimeric barcoding reagents by gravity and/or using centrifugation. The use of centrifugation for step (ii) (compared to relying on gravity alone) reduces the period during which the sample is manipulated prior to cell lysis and means that the period taken for this step is less dependent on the size and nature of specific cell types. A step of centrigugation may be performed at at least 50 G, at least 100 G, at least 200 G, at least 300 G, at least 500 G, at least 750 G, at least 1000 G, at least 1200 G, at least 1500 G, or at least 2000 G. A step of centrifugation may be performed for a duration of at least 5 seconds, at least 10 seconds, at least 30 seconds, at least 60 seconds, at least 5 minutes, at least 10 minutes or at least 30 minutes.

In the methods, step (a) (the step of contacting the sample with a library at least two multimeric barcoding reagents) may comprise mixing the sample with the library e.g. using a pipette. The mixing may be performed in a reaction vessel e.g. a tube or a well on a plate. The mixing may be facilated by shaking (e.g. 3D shaking), rotation and/or rocking (e.g. 2D rocking) of the reaction vessel. These approaches may provide more flexibility when scaling up or down the input numbers of cells and multimeric barcoding reagents as they are less dependent on the surface area of the vessel in use.

In step (a) (the step of contacting the sample with a library at least two multimeric barcoding reagents) the reaction vessel may be a tube or a well on a plate. The reaction vessel and/or any other plasticware used (e.g. a pipette) may be pre-coated with a substance (e.g. a polymer) to reduce non-specific adherence of the cells or the multimeric barcoding reagents to the surface of the reaction vessel and/or other plasticware. The polymer may be bovine serum albumin (BSA) and/or casein. A solution of BSA may be used to pre-coat the reaction vessel and/or any other plasticware used. The solution of BSA may be at least 0.1%, at least 0.3%, at least 0.5%, at least 0.8% or at least 1% w/v of BSA. Commercially available reaction vessels that may be used include Protein LoBind Tubes® (Eppendorf).

In the methods, the step of freezing the cells may be performed in dry ice, in a −80° C. freezer, in a −20° C. freezer, in a solvent cooling bath, or in liquid nitrogen.

In the methods, the step of freezing may be performed by exposing the cells to a temperature of less than −15° C., less than −20° C., less than −30° C., less than −40° C., less than −50° C., less than-50° C., less than −60° C., less than −70° C., less than −75° C., less than −80° C., less than −90° C., less than −100° C., less than −150° C., or less than −190° C.

In the methods, the step of freezing may be performed by exposing the cells to a temperature of approximately −15° C., approximately −20° C., approximately −30° C., approximately −40° C., approximately −50° C., approximately −50° C., approximately −60° C., approximately −70° C., approximately −75° C., approximately −80° C., approximately −100° C., approximately −150° C., or approximately −195° C.

In the methods, the step of freezing may be carried out for less than 0.5 seconds, less than 1 second, less than 2 seconds, less than 5 seconds, less than 10 seconds, less than 30 seconds, less than 1 minutes, less than 5 minutes, less than 10 minutes, less than 30 minutes, less than 1 hour, less than 6 hours, less than 12 hours or less than 24 hours.

In the methods, the step of freezing may be carried out for approximately 0.5 seconds, approximately 1 second, approximately 2 seconds, approximately 5 seconds, approximately 10 seconds, approximately 30 seconds, approximately 1 minutes, approximately 5 minutes, approximately 10 minutes, approximately 30 minutes, approximately 1 hour, approximately 6 hours, approximately 12 hours or approximately 24 hours.

In the methods, following the step of freezing, the cells may be maintained in a frozen state for at least 1 minute, at least 5 minutes, at least 10 minutes, at least 30 minutes, at least 1 hour, at least 1 day, at least 3 days, at least 7 days, at least 1 month, at least 6 months or at least 1 year.

In the methods, following the step of freezing, the cells may be maintained in a frozen state for approximately 1 minute, approximately 5 minutes, approximately 10 minutes, approximately 30 minutes, approximately 1 hour approximately 1 day, approximately 3 days, approximately 7 days, approximately 1 month, approximately 6 months or approximately 1 year.

The step of maintaining the cells in a frozen state may be performed in dry ice, in a −80° C. freezer, in a −20° C. freezer, in a solvent cooling bath, or in liquid nitrogen.

The step of maintaining the cells in a frozen state may be performed at less than −15° C., less than −20° C., less than −30° C., less than −40° C., less than −50° C., less than −50° C., less than −60° C., less than −70° C., less than −75° C., less than −80° C., less than −100° C., less than −150° C., or less than-190° C.

The step of maintaining the cells in a frozen state may be performed at approximately −15° C., approximately −20° C., approximately −30° C., approximately −40° C., approximately −50° C., approximately −50° C., approximately −60° C., approximately −70° C., approximately −75° C., approximately −80° C., approximately −100° C., approximately −150° C., or approximately −195° C.

In the methods the cells may be maintained in a frozen state at a temperature of less than −15° C. for at least 1 minute, at least 5 minutes, at least 10 minutes, at least 30 minutes, at least 1 hour, at least 1 day, at least 3 days, at least 7 days, at least 1 month, at least 6 months or at least 1 year.

In the methods the cells may be maintained in a frozen state at a temperature of less than −70° C. for at least 1 minute, at least 5 minutes, at least 10 minutes, at least 30 minutes, at least 1 hour, at least 1 day, at least 3 days, at least 7 days, at least 1 month, at least 6 months or at least 1 year.

In the methods the cells may be maintained in a frozen state at a temperature of less than −75° C. for at least 1 minute, at least 5 minutes, at least 10 minutes, at least 30 minutes, at least 1 hour, at least 1 day, at least 3 days, at least 7 days, at least 1 month, at least 6 months or at least 1 year.

In the methods, the step of thawing the cells may be carried out by exposing the cells to a temperature of at least 4° C., at least 10° C., at least 20° C., at least 25° C., at least 30° C., at least 37° C., at least 40° C., at least 45° C., at at least 50° C., at least 55° C., at least 60° C., at least 65° C., at least 70° C., at least 75° C., or at least 80° C. This step may be performed at a fixed temperature or by using a temperature gradient (i.e. comprising multiple and/or changing temperatures over a period of time).

A controlled, rapid lysis can facilitate annealing (or ligation) of barcoded oligonucleotides to sub-sequences of target nucleic acids and thereby increases the likelihood of barcoded oligonucleotides of a single multimeric barcoding reagent annealing (or ligating) to sub-sequences of a target nucleic acid (e.g. mRNA) from a single cell (for example, rather than not annealing or ligating to target nucleic acids at all, and/or rather than annealing or ligating to sub-sequences from more than one cell). Therefore, a rapid thaw step that follows the freezing step can produce more efficient and high-fidelity cell lysis due to the rapid temperature increase.

In the methods, the step of thawing the cells may be carried out for at least 5 seconds, at least 10 seconds, at least 20 seconds, at least 30 seconds, at least 40 seconds, at least 50 seconds, at least 1 minute, at least 5 minutes, or at least 10 minutes.

In the methods, the step of thawing the cells may be carried out by exposing the cells to a temperature of at at least 55° C. for at least 5 seconds, at least 10 seconds, at least 20 seconds, at least 30 seconds, at least 40 seconds, at least 50 seconds, at least 1 minute, at least 5 minutes or at least 10 minutes.

In the methods, the step of thawing the cells may be carried out by exposing the cells to a temperature of at at least 60° C. for at least 5 seconds, at least 10 seconds, at least 20 seconds, at least 30 seconds, at least 40 seconds, at least 50 seconds, at least 1 minute, at least 5 minutes or at least 10 minutes.

The method may further comprise (d) capturing the barcoded oligonucleotides and/or barcoded target nucleic acid molecules and/or multimeric barcoding reagents on a solid support.

In the methods, the target nucleic acids may be mRNA and step (d) may comprise capturing barcoded oligonucleotides appended (e.g. annealed or ligated) to sub-sequences of mRNA, and wherein the method further comprises (e) reverse transcription of mRNA to generate cDNA. The method may further comprise amplification of the generated cDNA (e.g. by PCR). A single-stranded DNA-binding protein may be added to the reverse transcription reaction. Such a single-stranded DNA-binding protein may destabilise helical duplexes and allow enzymes to access their substrates more easily, reduce single stranded DNA secondary structure and/or protect single stranded DNA products from nucleases.

The solid support may be any solid support as described herein (e.g. beads).

In the method, the solid support may comprise streptavidin moieties and the barcoded oligonucleotides and/or barcoded target nucleic acid molecules and/or multimeric barcoding reagents may be captured on the solid support through streptavidin-biotin interaction.

The method may comprise contacting the sample with the solid support in step (a), (b), (c) and/or (d). Preferably, the sample is contacted with the solid support prior to step (c). In such methods, the steps of annealing or ligating barcoded oligonucleotides to sub-sequences of target nucleic acids to produce barcoded target nucleic acid molecules (step (c)) and capturing the barcoded oligonucleotides and/or barcoded target nucleic acid molecules and/or multimeric barcoding reagents on a solid support (step (d)) may be performed simultaneously. The simultaneous performance of steps (c) and (d) (e.g. barcoding and capture of of target mRNA molecules) may be performed at a single temperature or across a gradient of temperatures.

The method may comprise one or more steps of diluting the cells. This may enable more efficient appending (e.g. annealing or ligation) of barcoded oligonucleotides to sub-sequences of a target nucleic acid.

The method may comprise performing step (a), (b), (c), (d) and/or (e) in the presence of an RNA stabilising molecule. The RNA stabilising agent may be an RNA carrier. The RNA carrier may be bovine serum albumin (BSA), transfer RNA (tRNA) (e.g. from bacteria or yeast), glycogen, and/or linear polyacrylamide (LPA). The use of one or more of these agents may improve the capture of barcoded oligonucleotides annealed to sub-sequences of target nucleic acid (e.g. mRNA).

The method may comprise performing step (a), (b), (c), (d) and/or (e) in the presence of a protic or aprotic solvent. The solvent may be A-butyrolactone, 8-valerolactam, 2-pyrrolidone, formamide, ethylene carbonate and/or propylene carbonate. The solution (comprising the multimeric barcoding reagents and/or cells) may comprise at least 1%, at least 5%, at least 10%, at least 20%, or at least 50% by weight or by volume of one or more of the solvents. These solvents may improve cell lysis but also lower the melting temperature of a hybridisation reaction and reduce secondary structure amongst target nucleic acid molecules (e.g. RNA molecules). In addition they may facilitate the release of barcoded oligonucleotides from the multimeric barcoding reagents (as the T_{M} is lowered and so the barcoded oligonucleotides are released) and/or the annealing of the barcoded oligonucleotides to the sub-sequences of the target nucleic acid (e.g. mRNA molecules).

The method may comprise performing step (a), (b), (c), (d) and/or (e) in the presence of a molecular crowding agent. The molecular crowding agent may be a poly(ethylene) glycol (PEG) solution (e.g. PEG 600, PEG 2000, PEG 4000, PEG 6000, PEG 8000, PEG 10,000, PEG 20,000, PEG 35,000 and/or PEG 40,000). Optionally, the solution (comprising the multimeric barcoding reagents and/or cells) may comprise at least 1% poly(ethylene) glycol, at least 5% poly(ethylene) glycol, at least 10% poly(ethylene) glycol, or at least 20% poly(ethylene) glycol by weight or by volume. Optionally, such a high-viscosity solution may be comprised of a polyvinylpyrrolidone (PVP) solution, such as PVP 10,000 or PVP 20,000 or PVP 35,000. Optionally, such a solution may comprise at least 1% PVP, at least 5% PVP, at least 10% PVP, or at least 20% PVP by weight or by volume. Optionally, such a solution may be comprised of a dextran solution, such as dextran 5000. Optionally, such a solution may comprise at least 1% dextran, at least 5% dextran, at least 10% dextran, or at least 20% dextran by weight or by volume. Optionally, such a high-viscosity solution may be comprised of a polyvinyl acetate (PVA) or a polyacryclic acid (PAA) solution, such as PVA 10,000 or PAA 8,000. Optionally, such a solution may comprise at least 1% PVA or PAA, at least 5% PVA or PAA, at least 10% PVA or PAA, or at least 20% dextran by weight or by volume. Optionally, such a solution may be comprised of glycerol. Optionally, such a solution may comprise at least 1% glycerol, at least 5% glycerol, at least 10% glycerol, or at least 20% glycerol by volume. Molecular crowding agents may increase the efficiency and specificity of the reactions.

In the methods, step (a), (b), (c), (d) and/or (e) may be performed in a high-viscosity solution. The high-viscosity solution may have a dynamic viscosity of at least 1.0 centipoise, at least 1.1 centipoise, at least 1.2 centipoise, at least 1.5 centipoise, at least 2.0 centipoise, at least 5.0 centipoise, at least 10.0 centipoise, at least 20.0 centipoise, at least 50.0 centipoise, at least 100.0 centipoise, or at least 200.0 centipoise (wherein such respective dynamic viscosities are at 25 degrees Celsius at standard sea-level pressure). Preferably, the high-viscosity solution has a dynamic viscosity of at least 2.0 centipoise.

The invention provides a method of preparing first and second nucleic acid samples for sequencing, wherein each sample comprises at least 2 cells, and wherein the method comprises performing for each sample steps (a), (b) and (c), and optionally steps (d) and/or (e), as defined in any of the methods described herein. Step (a) may be performed at a different timepoint for the first and second nucleic acid samples. The step of freezing the cells may be performed at a different timepoint for the first and second nucleic acid samples. The cells of the first nucleic acid sample may be maintained in a frozen state for a different duration of time relative to the duration of time for which the cells of the second nucleic acid sample are maintained in a frozen state.

The difference between the duration of time for which the cells of the first nucleic acid sample are maintained in a frozen state and the duration of time for which the cells of the second nucleic acid sample are maintained in a frozen state may be at least 5 minutes, at least 30 minutes, at least 1 hour, at least 6 hours, at least 12 hours, at least 24 hours, at least 7 days, at least 1 month, at least 6 months or at least 1 year. In the methods, step (c), and optionally step (d) and/or step (e), may be performed within a single contiguous 24-hour period for both the first and second nucleic acid samples.

The invention provides a multimeric barcoding reagent for labelling a target nucleic acid for sequencing, wherein the multimeric barcoding reagent comprises:
a. a support;
b. at least two multimeric hybridization molecules, wherein each multimeric hybridization molecule is independently linked to the support and wherein each multimeric hybridization molecule comprises at least two hybridization molecules linked together, wherein each of the hybridization molecules comprises a nucleic acid sequence comprising a hybridization region;
c. at least two barcoded oligonucleotides annealed to each of the multimeric hybridization molecules, wherein each barcoded oligonucleotide is annealed to one of the hybridization regions and wherein each barcoded oligonucleotide comprises a barcode region; and
d. a cell-binding moiety linked to each multimeric hybridization molecule.

The hybridization molecules of each multimeric hybridization molecule may be linked on a nucleic acid molecule.

The hybridization molecules of each multimeric hybridization molecule may be linked on a linear nucleic acid molecule.

The first end of each linear nucleic acid molecule may be linked to the support and the second end is linked to a cell-binding moiety.

Each cell-binding moiety may be linked to one of the multimeric hybridization molecules by a cell-binding oligonucleotide.

Each cell-binding oligonucleotide may be annealed to one of the multimeric hybridization molecules.

Each barcoded oligonucleotide may comprise, optionally in the 5′ to 3′ direction, an adapter region annealed to one of the hybridization regions, a barcode region, and a target region capable of annealing or ligating to a sub-sequence of the target nucleic acid.

Each barcoded oligonucleotide may comprise, optionally in the 5′ to 3′ direction, a barcode region, an adapter region annealed to one of the hybridization regions and a target region capable of annealing or ligating to a sub-sequence of the target nucleic acid.

The adapter regions of the barcoded oligonucleotides of the multimeric barcoding reagent may be identical.

Each multimeric hybridization molecule may comprise at least 2, at least 3, at least 5, at least 10, at least 20, at least 50, at least 100, at least 200, at least 500, at least 1000, at least 5000, at least 10⁴, at least 10⁵, at least 10⁶, at least 10⁷, at least 10⁸, at least 10⁹ or at least 10¹⁰ hybridization molecules linked together, wherein each of the hybridization molecules comprises a nucleic acid sequence comprising a hybridization region.

The multimeric barcoding reagent may comprise a barcoded oligonucleotide for each of the hybridization regions, and wherein each barcoded oligonucleotide is annealed to one of the hybridization regions.

The multimeric barcoding reagent may comprise at least 2, at least 3, at least 5, at least 10, at least 20, at least 50, at least 100, at least 200, at least 500, at least 1000, at least 5000, at least 10⁴, at least 10⁵, at least 10⁶, at least 10⁷, at least 10⁸, at least 10⁹, or at least 10¹⁰ barcoded oligonucleotides annealed to each of the multimeric hybridization molecules, wherein each barcoded oligonucleotide is annealed to one of the hybridization regions and wherein each barcoded oligonucleotide comprises a barcode region.

The multimeric barcoding reagent may comprise at least 2, at least 3, at least 5, at least 10, at least 20, at least 50, at least 100, at least 200, at least 500, at least 1000, at least 5000, at least 10⁴, at least 10⁵, at least 10⁶, at least 10⁷, at least 10⁸, at least 10⁹, or at least 10¹⁰ barcoded oligonucleotides with identical barcode regions.

The multimeric barcoding reagent may comprise at least 3, at least 4, at least 5, at least 10, at least 20, at least 25, at least 50, at least 75, at least 100, at least 200, at least 500, at least 1000, at least 5000, at least 10⁴, at least 10⁵, at least 10⁶, at least 10⁷, at least 10⁸, at least 10⁹, or at least 10¹⁰ multimeric hybridization molecules.

The invention provides a library of multimeric barcoding reagents comprising at least 2, at least 5, at least 10, at least 20, at least 25, at least 50, at least 75, at least 100, at least 250, at least 500, at least 10³, at least 10⁴, at least 10⁵, at least 10⁶, at least 10⁷, at least 10⁸, at least 10⁹, at least 10¹⁰ multimeric barcoding reagents.

In the library of multimeric barcoding reagents, at least 50%, at least 60%, at least 70%, at least 75%, at least 80%, at least 85%, at least 90%, at least 95%, at least 99%, at least 99.9%, at least 99.99%, at least 99.999%, at least 99.9999%, or 100% of the barcode regions of each multimeric barcoding reagent may be different to the barcode regions of the other multimeric barcoding reagents in the library.

The invention provides a method of preparing a nucleic acid sample for sequencing, wherein the sample comprises at least 2 cells, and wherein the method comprises in order the steps of:
(a) contacting the sample with a library comprising at least two multimeric barcoding reagents, wherein the barcode regions of the barcoded oligonucleotides of a first multimeric barcoding reagent of the library are different to the barcode regions of the barcoded oligonucleotides of a second multimeric barcoding reagent of the library, wherein the cell-binding moiety of the first multimeric barcoding reagent binds to the cell membrane of a first cell prior to step (b), and wherein the cell-binding moiety of the second multimeric barcoding reagent binds to the cell membrane of a second cell prior to step (b);
(b) lysing the cells or permeabilizing the cell membranes of the cells; and
(c) (separately) annealing or ligating each of the barcoded oligonucleotides of the first multimeric barcoding reagent to at least four sub-sequences of a target nucleic acid of the first cell to produce at least four barcoded target nucleic acid molecules, and (separately) annealing or ligating each of the barcoded oligonucleotides of the second multimeric barcoding reagent to a least four sub-sequences of a target nucleic acid of the second cell to produce at least four barcoded target nucleic acid molecules, optionally wherein the cells are comprised within a single contiguous aqueous volume during steps (a), (b) and (c).

In the method, step (c) may comprise:
(i) annealing each of the barcoded oligonucleotides of the first multimeric barcoding reagent to at least four sub-sequences of a target nucleic acid of the first cell, and annealing each of the barcoded oligonucleotides of the second multimeric barcoding reagent to at least four sub-sequences of a target nucleic acid of the second cell; and
(ii) extending each of the barcoded oligonucleotides of the first multimeric barcoding reagent to produce at least four different barcoded target nucleic acid molecules and extending each of the barcoded oligonucleotides of the second multimeric barcoding reagent to produce at least four different barcoded target nucleic acid molecules, wherein each of the barcoded target nucleic acid molecules comprises at least one nucleotide synthesised from the target nucleic acid as a template.

In the method, the target nucleic acids may be mRNA.

The invention provides a method of synthesising a multimeric barcoding reagent for labelling a target nucleic acid, wherein the method comprises:
a. synthesizing a library of barcoded oligonucleotides by amplifying a plurality of unique oligonucleotides, wherein each of the plurality of unique oligonucleotides comprises a barcode region and at least one constant region;
b. contacting the library of barcoded oligonucleotides with at least two multimeric hybridization molecules, wherein each multimeric hybridization molecule is independently linked to a single support and wherein each multimeric hybridization molecule comprises at least two hybridization molecules linked together, wherein each of the hybridization molecules comprises a nucleic acid sequence comprising a hybridization region; and
c. forming the multimeric barcoding reagent by annealing at least two barcoded oligonucleotides of the library of barcoded oligonucleotides to each of the multimeric hybridization molecules, wherein each barcoded oligonucleotide is annealed to one of the hybridization regions and wherein each barcoded oligonucleotide comprises a barcode region;
and optionally wherein steps (a), (b) and (c) are performed in a single contiguous aqueous volume.

Preferably the support is a bead e.g. a magnetic bead. The support may be any of the supports described herein.

Each of the plurality of unique oligonucleotides may comprise in the 5′ to 3′ direction, a 5′ constant region, a barcode region and a 3′ constant region, and optionally wherein step (a) comprises amplifying each of the plurality of unique oligonucleotides using a pair of primers that anneal to the 5′ constant region and the 3′ constant region.

The plurality of unique oligonucleotides may comprise at least 2, at least 5, at least 10, at least 20, at least 50, at least 100, at least 10³, at least 10⁴, at least 10⁵, at least 10⁶, at least 10⁷ or at least 10⁸ unique oligonucleotides with unique barcode regions.

Step (b) may comprise contacting the library of barcoded oligonucleotides with at least 5, at least 10, at least 20, at least 50, at least 100, at least 10³, at least 10⁴, at least 10⁵, at least 10⁶, at least 10⁷, at least 10⁸, at least 10⁹ or at least 10¹⁰ multimeric hybridization molecules, wherein each multimeric hybridization molecule is independently linked to the same single support. Preferably, step (b) comprises contacting the library of barcoded oligonucleotides with at least 10⁴ multimeric hybridization molecules, wherein each multimeric hybridization molecule is independently linked to the same single support.

Each multimeric barcoding reagent may be formed by annealing at least 3, at least 5, at least 10, at least 20, at least 50, at least 100, at least 10³, at least 10⁴, at least 10⁵, at least 10⁶, at least 10⁷, at least 10⁸, at least 10⁹ or at least 10¹⁰ barcoded oligonucleotides to each of the multimeric hybridization molecules. Preferably, each multimeric barcoding reagent is formed by annealing at least 3 barcoded oligonucleotides to each of the multimeric hybridization molecules.

Each multimeric barcoding reagent may be formed by annealing at least 5, at least 10, at least 20, at least 50, at least 100, at least 10³, at least 10⁴, at least 10⁵, at least 10⁶, at least 10⁷ or at least 10⁸, or at least 10⁹ barcoded oligonucleotides to the multimeric hybridization molecules that are independently linked to the same single support. Preferably, each multimeric barcoding reagent is formed by annealing at least 10⁵ barcoded oligonucleotides to the multimeric hybridization molecules that are independently linked to the same single support.

Each multimeric barcoding reagent may be formed by annealing at least 2, at least 3, at least 4, at least 5, at least 10, at least 20, at least 50, at least 100, at least 10³, at least 10⁴, at least 10⁵, at least 10⁶, at least 10⁷ or at least 10⁸ unique barcoded oligonucleotides to each of the multimeric hybridization molecules. Preferably, each multimeric barcoding reagent is formed by annealing at least 3 unique barcoded oligonucleotides to each of the multimeric hybridization molecules. Each multimeric barcoding reagent may be formed by annealing at least 2, at least 5, at least 10, at least 20, at least 50, at least 100, at least 10³, at least 10⁴, at least 10⁵, at least 10⁶, at least 10⁷ or at least 10⁸ copies of each unique barcoded oligonucleotide to each of the multimeric hybridization molecules. Preferably, each multimeric barcoding reagent is formed by annealing at least 3 copies of each unique barcoded oligonucleotide to each of the multimeric hybridization molecules.

Each multimeric barcoding reagent may be formed by annealing at least 5, at least 10, at least 20, at least 50, at least 100, at least 1000, at least 5000, at least 10⁴, at least 10⁵, at least 10⁶, at least 10⁷ or at least 10⁸ unique barcoded oligonucleotides to the multimeric hybridization molecules that are independently linked to the same single support. Preferably, each multimeric barcoding reagent is formed by annealing at least 10 unique barcoded oligonucleotides to the multimeric hybridization molecules that are independently linked to the same single support. Each multimeric barcoding reagent may be formed by annealing at least 2, at least 5, at least 10, at least 20, at least 50, at least 100, at least 10³, at least 10⁴, at least 10⁵, at least 10⁶, at least 10⁷ or at least 10⁸ copies of each unique barcoded oligonucleotide to the multimeric hybridization molecules that are independently linked to the same single support. Preferably, each multimeric barcoding reagent is formed by annealing at least 10⁴ copies of each unique barcoded oligonucleotide to the multimeric hybridization molecules that are independently linked to the same single support.

The method may comprise performing in parallel any of the methods described herein in at least two, at least 5, at least 10, at least 100, at least 10³, at least 10⁴, at least 10⁵, at least 10⁶, at least 10⁷ or at least 10⁸ physically separate single contiguous aqueous volumes, optionally wherein each physically separate single contiguous aqueous volume is in a separate well.

The method may further comprise pooling together the physically separate single contiguous aqueous volumes comprising multimeric barcoding reagents to form the library of multimeric barcoding reagents.

In the method, at least 50%, at least 60%, at least 70%, at least 75%, at least 80%, at least 85%, at least 90%, at least 95%, at least 99%, at least 99.9%, at least 99.99%, at least 99.999%, at least 99.9999%, or 100% of the barcode regions of each multimeric barcoding reagent synthesized in each physically separate single contiguous aqueous volume may be different to the barcode regions of the multimeric barcoding reagents formed in the other physically separate single contiguous aqueous volumes.

The method may further comprise sequencing the library of barcoded oligonucleotides in each physically separate single contiguous aqueous volume to generate a profile of the barcode regions of the barcoded oligonucleotides in each physically separate single contiguous aqueous volume, optionally wherein the step of sequencing is performed after step (a) and before step (b).

The invention provides a library comprising at least two multimeric barcoding reagents, wherein each multimeric barcoding reagent comprises: (a) first and second barcoded oligonucleotides linked together and a cell-binding moiety, wherein the barcoded oligonucleotides each comprise a barcode region and wherein the barcode regions of the first and second barcoded oligonucleotides of a first multimeric barcoding reagent of the library are different to the barcode regions of the first and second barcoded oligonucleotides of a second multimeric barcoding reagent of the library.

The invention provides a library of multimeric barcoding reagents comprising at least 2 multimeric barcoding reagents for labelling target nucleic acids for sequencing, wherein each multimeric barcoding reagent comprises: (a) first and second hybridization molecules linked together, wherein each of the hybridization molecules comprises a nucleic acid sequence comprising a hybridization region; (b) first and second barcoded oligonucleotides, wherein the first barcoded oligonucleotide is annealed to the hybridization region of the first hybridization molecule and wherein the second barcoded oligonucleotide is annealed to the hybridization region of the second hybridization molecule, wherein the barcoded oligonucleotides each comprise a barcode region; and (c) a cell-binding moiety; wherein the barcode regions of the first and second barcoded oligonucleotides of a first multimeric barcoding reagent of the library are different to the barcode regions of the first and second barcoded oligonucleotides of a second multimeric barcoding reagent of the library.

A cell-binding moiety may be attached to each of the barcode molecules. Additionally or alternatively, a cell-binding moiety may be attached to each of the barcoded oligonucleotides.

The multimeric barcoding reagents may be for labelling sub-sequences of a target nucleic acid in a cell.

Each multimeric barcoding reagent in the library may be for labelling the target nucleic acids of a single cell. Each multimeric barcoding reagent in the library may be for labelling the target nucleic acids in a single cell.

The first and second hybridization molecules may be comprised within a (single) nucleic acid molecule. Alternatively, the first and second hybridization molecules may be linked together by a support e.g. a macromolecule, solid support or semi-solid support, as described herein.

The first and second barcoded oligonucleotides may take any form described herein. For example, each barcoded oligonucleotide may further comprise a target region.

The library may comprise at least 10 multimeric barcoding reagents. The barcode regions of the first and second barcoded oligonucleotides of each multimeric barcoding reagent may be different to the barcode regions of the barcoded oligonucleotides of at least 9 other multimeric barcoding reagents in the library.

The invention provides a library of multimeric barcoding reagents comprising at least 10 multimeric barcoding reagents for labelling target nucleic acids for sequencing, wherein each multimeric barcoding reagent comprises: (a) first and second hybridization molecules comprised within a nucleic acid molecule, wherein each of the hybridization molecules comprises a nucleic acid sequence comprising a hybridization region; (b) first and second barcoded oligonucleotides, wherein the first barcoded oligonucleotide is annealed to the hybridization region of the first hybridization molecule and wherein the second barcoded oligonucleotide is annealed to the hybridization region of the second hybridization molecule, wherein the barcoded oligonucleotides each comprise a barcode region; and (c) a cell-binding moiety; wherein the barcode regions of the first and second barcoded oligonucleotides of each multimeric barcoding reagent of the library are different to the barcode regions of the barcoded oligonucleotides of at least 9 other multimeric barcoding reagents of the library.

The library may comprise at least two multimeric barcoding reagents each comprising: (a) first and second barcode molecules linked together, wherein each of the barcode molecules comprises a nucleic acid sequence comprising a barcode region; (b) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule; and (c) a cell-binding moiety; wherein the barcode regions of the first and second barcoded oligonucleotides of a first multimeric barcoding reagent of the library are different to the barcode regions of the first and second barcoded oligonucleotides of a second multimeric barcoding reagent of the library.

A cell-binding moiety may be attached to each of the barcode molecules. Additionally or alternatively, a cell-binding moiety may be attached to each of the barcoded oligonucleotides.

The library may comprise at least 10 multimeric barcoding reagents, wherein each multimeric barcoding reagent comprises: (a) first and second barcode molecules comprised within a nucleic acid molecule, wherein each of the barcode molecules comprises a nucleic acid sequence comprising a barcode region; (b) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule; and (c) a cell-binding moiety; wherein the barcode regions of the first and second barcoded oligonucleotides of each multimeric barcoding reagent of the library are different to the barcode regions of the barcoded oligonucleotides of at least 9 other multimeric barcoding reagents of the library.

In the libraries, each multimeric barcoding reagent may be comprised within a different (or separate) lipid carrier. The lipid carrier may be a micelle or a liposome. Alternatively, the lipid carrier may take any of the forms described herein.

The invention provides a kit for labelling target nucleic acids for sequencing, wherein the kit comprises: (a) a library of multimeric barcoding reagents comprising at least two multimeric barcoding reagents, wherein each multimeric barcoding reagent comprises (i) first and second barcode molecules linked together, wherein each of the barcode molecules comprises a nucleic acid sequence comprising, optionally in the 5′ to 3′ direction, an adapter region and a barcode region, (ii) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule; wherein the barcode regions of the first and second barcoded oligonucleotides of a first multimeric barcoding reagent of the library are different to the barcode regions of the first and second barcoded oligonucleotides of a second multimeric barcoding reagent of the library; and (b) first and second adapter oligonucleotides for each of the multimeric barcoding reagents, wherein the first adapter oligonucleotide comprises an adapter region capable of annealing to the adapter region of the first barcode molecule and wherein the second adapter oligonucleotide comprises an adapter region capable of annealing to the adapter region of the second barcode molecule, and wherein a cell-binding moiety is attached to each of the adapter oligonucleotides.

The kit may be for labelling target nucleic acids of (or in) at least two cells for sequencing.

The multimeric barcoding reagents may each comprise a cell-binding moiety. A cell-binding moiety may be attached to each of the barcode molecules. A cell-binding moiety may be attached to each of the barcoded oligonucleotides.

The invention provides a kit for labelling target nucleic acids for sequencing, wherein the kit comprises: (a) a library of multimeric barcoding reagents comprising at least two multimeric barcoding reagents, wherein each multimeric barcoding reagent comprises first and second barcoded oligonucleotides linked by a support, wherein the barcoded oligonucleotides each comprise a barcode region and a target region, and wherein the barcode regions of the first and second barcoded oligonucleotides of a first multimeric barcoding reagent of the library are different to the barcode regions of the first and second barcoded oligonucleotides of a second multimeric barcoding reagent of the library; and (b) a cell-binding moiety for each multimeric barcoding reagent in the library, wherein each such cell-binding moiety is capable of binding to a multimeric barcoding reagent within the library

The invention provides a kit for labelling target nucleic acids for sequencing, wherein the kit comprises: (a) a library of multimeric barcoding reagents comprising at least two multimeric barcoding reagents, wherein each multimeric barcoding reagent comprises at least first and second barcoded oligonucleotides linked by a support, wherein the barcoded oligonucleotides each comprise a barcode region and a poly(T) target region, and wherein the barcode regions of the first and second barcoded oligonucleotides of a first multimeric barcoding reagent of the library are different to the barcode regions of the first and second barcoded oligonucleotides of a second multimeric barcoding reagent of the library; and (b) a cell-binding moiety for each multimeric barcoding reagent in the library, wherein each such cell-binding moiety is capable of binding to a multimeric barcoding reagent within the library

The invention provides a kit for labelling target nucleic acids for sequencing, wherein the kit comprises: (a) a library of multimeric barcoding reagents comprising at least two multimeric barcoding reagents, wherein each multimeric barcoding reagent comprises at least first and second barcoded oligonucleotides linked by a support, wherein the barcoded oligonucleotides each comprise a barcode region and a target region, and wherein the barcode regions of the first and second barcoded oligonucleotides of a first multimeric barcoding reagent of the library are different to the barcode regions of the first and second barcoded oligonucleotides of a second multimeric barcoding reagent of the library; (b) a cell-binding moiety for each multimeric barcoding reagent in the library, wherein each such cell-binding moiety is capable of binding to a multimeric barcoding reagent within the library; and (c) blocking oligonucleotides (e.g. a solution of blocking oligonucleotides), wherein each blocking oligonucleotide comprises a sequence complementary to all or part of a barcoded oligonucleotide, and/or comprises a sequence complementary to all or part of a target nucleic acid.

The invention provides a kit for labelling target nucleic acids for sequencing, wherein the kit comprises: (a) a library of multimeric barcoding reagents comprising at least two multimeric barcoding reagents, wherein each multimeric barcoding reagent comprises at least first and second barcoded oligonucleotides linked by a support, wherein the barcoded oligonucleotides each comprise a barcode region and a poly(T) target region, and wherein the barcode regions of the first and second barcoded oligonucleotides of a first multimeric barcoding reagent of the library are different to the barcode regions of the first and second barcoded oligonucleotides of a second multimeric barcoding reagent of the library; (b) a cell-binding moiety for each multimeric barcoding reagent in the library, wherein each such cell-binding moiety is capable of binding to a multimeric barcoding reagent within the library; and (c) blocking oligonucleotides (e.g. a solution of blocking oligonucleotides), wherein each blocking oligonucleotide comprises a sequence complementary to all or part of a barcoded oligonucleotide, and/or comprises a sequence complementary to all or part of a target nucleic acid.

In any kit comprising a library of multimeric barcoding reagents and cell-binding moieties, two or more cell-binding moieties may be provided (e.g. in a solution of cell-binding moieties) separately to a library of multimeric barcoding reagents (e.g. a solution of a library of multimeric barcoding reagents).

In any kit comprising a library of multimeric barcoding reagents and cell-binding moieties, the library of multimeric barcoding reagents and the cell-binding moieties may be provided together in a single solution.

In any kit comprising a library of multimeric barcoding reagents, cell-binding moieties and blocking oligonucleotides, each of the three components of the kit may be provided separately (e.g. in a separate solution) to the other two components of the kit. Optionally, two components of the kit may be provided together (e.g. in a single solution). Optionally, all three components of the kit may be provided together (e.g. in a single solution).

The invention provides a kit for labelling target nucleic acids for sequencing, wherein the kit comprises: (a) a library of multimeric barcoding reagents comprising at least 10 multimeric barcoding reagents, wherein each multimeric barcoding reagent comprises (i) first and second barcode molecules comprised within a nucleic acid molecule, wherein each of the barcode molecules comprises a nucleic acid sequence comprising, optionally in the 5′ to 3′ direction, an adapter region and a barcode region, (ii) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule; wherein the barcode regions of the first and second barcoded oligonucleotides of each multimeric barcoding reagent of the library are different to the barcode regions of the barcoded oligonucleotides of at least 9 other multimeric barcoding reagent of the library; and (b) first and second adapter oligonucleotides for each of the multimeric barcoding reagents, wherein the first adapter oligonucleotide comprises an adapter region capable of annealing to the adapter region of the first barcode molecule and wherein the second adapter oligonucleotide comprises an adapter region capable of annealing to the adapter region of the second barcode molecule, and wherein a cell-binding moiety is attached to each of the adapter oligonucleotides.

In the kits, the adapter oligonucleotides for each multimeric barcoding reagent may be comprised within a different (or separate) lipid carrier. The lipid carrier may be a micelle or a liposome. Alternatively, the lipid carrier may take any of the forms described herein. The lipid carriers may each further comprise a multimeric barcoding reagent e.g. the first lipid carrier comprises the first multimeric barcoding reagent and the adapter oligonucleotides for the first multimeric barcoding reagent.

In the libraries or kits, the barcoding reagents may each comprise a solid support or semi-solid support, and wherein a cell-binding moiety is attached to the solid support or semi-solid support (e.g. by a covalent or non-covalent bond).

The invention provides a method of preparing a nucleic acid sample for sequencing, wherein the sample comprises a cell, and wherein the method comprises the steps of: (a) contacting the sample with a multimeric barcoding reagent, wherein the multimeric barcoding reagent comprises first and second barcode regions linked together and a cell-binding moiety, wherein each barcode region comprises a nucleic acid sequence, wherein the cell-binding moiety of the multimeric barcoding reagent binds to the cell membrane of the cell and the first and second barcode regions of the multimeric barcoding reagent are internalized into the cell; and
(b) appending barcode sequences to each of the first and second sub-sequences of a target nucleic acid of the cell to produce first and second barcoded target nucleic acid molecules for the cell, wherein the first barcoded target nucleic acid molecule comprises the nucleic acid sequence of the first barcode region of the multimeric barcoding reagent and the second barcoded target nucleic acid molecule comprises the nucleic acid sequence of the second barcode region of the multimeric barcoding reagent.

The invention provides a method of preparing a nucleic acid sample for sequencing, wherein the sample comprises at least two cells (e.g. a cell from a cell line, or a cell originating from blood, or a cell originating from a tissue or organ sample, or a cell originating from a pre-implantation embryo generated by in vitro fertilisation), wherein the cell contains at least two fragments of a target nucleic acid (e.g. genomic DNA, and/or messenger RNA), and wherein the method comprises the steps of: (a) contacting the sample with a library comprising at least two multimeric barcoding reagents, wherein each multimeric barcoding reagent comprises first and second barcode regions linked together and a cell-binding moiety, wherein each barcode region comprises a nucleic acid sequence and wherein the first and second barcode regions of a first multimeric barcoding reagent are different to the first and second barcode regions of a second multimeric barcoding reagent of the library, wherein the cell-binding moiety of the first multimeric barcoding reagent from the library binds to the cell membrane of a first cell of the sample and the first and second barcode regions of the first multimeric barcoding reagent are internalized into the first cell, and wherein the cell-binding moiety of the second multimeric barcoding reagent from the library binds to the cell membrane of a second cell of the sample and the first and second barcode regions of the second multimeric barcoding reagent are internalized into the second cell; and
(b) appending barcode sequences to each of first and second sub-sequences of a target nucleic acid of the first cell to produce first and second barcoded target nucleic acid molecules for the first cell, wherein the first barcoded target nucleic acid molecule comprises the nucleic acid sequence of the first barcode region of the first multimeric barcoding reagent and the second barcoded target nucleic acid molecule comprises the nucleic acid sequence of the second barcode region of the first multimeric barcoding reagent, and appending barcode sequences to each of first and second sub-sequences of a target nucleic acid of the second cell to produce first and second barcoded target nucleic acid molecules from the second cell, wherein the first barcoded target nucleic acid molecule comprises the nucleic acid sequence of the first barcode region of the second multimeric barcoding reagent and the second barcoded target nucleic acid molecule comprises the nucleic acid sequence of the second barcode region of the second multimeric barcoding reagent.

The method may comprise the steps of: (a) contacting the sample with a library comprising first and second multimeric barcoding reagents, wherein each multimeric barcoding reagent comprises first and second barcode molecules linked together and a cell-binding moiety, wherein each of the barcode molecules comprises a nucleic acid sequence comprising a barcode region and an adapter region and wherein the first and second barcode regions of a first multimeric barcoding reagent are different to the first and second barcode regions of a second multimeric barcoding reagent of the library, and wherein the cell-binding moiety of the first multimeric barcoding reagent from the library binds to the cell membrane of a first cell of the sample and the first and second barcode molecules of the first multimeric barcoding reagent are internalized into the first cell, and wherein the cell-binding moiety of the second multimeric barcoding reagent from the library binds to the cell membrane of a second cell of the sample and the first and second barcode molecules of the second multimeric barcoding reagent are internalized into the second cell; (b) appending a coupling sequence to each of first and second sub-sequences of a target nucleic acid of a first cell, and appending a coupling sequence to each of first and second sub-sequences of a target nucleic acid of a second cell; (c) for each of the multimeric barcoding reagents, annealing the coupling sequence of the first sub-sequence to the adapter region of the first barcode molecule, and annealing the coupling sequence of the second sub-sequence to the adapter region of the second barcode molecule; and (d) appending barcode sequences to each of the first and second sub-sequences of the target nucleic acid of the first cell to produce first and second barcoded target nucleic acid molecules for the first cell, wherein the first barcoded target nucleic acid molecule comprises the nucleic acid sequence of the barcode region of the first barcode molecule of the first multimeric barcoding reagent and the second barcoded target nucleic acid molecule comprises the nucleic acid sequence of the barcode region of the second barcode molecule of the first multimeric barcoding reagent, and appending barcode sequences to each of the first and second sub-sequences of a target nucleic acid of the second cell to produce first and second barcoded target nucleic acid molecules from the second cell, wherein the first barcoded target nucleic acid molecule comprises the nucleic acid sequence of the barcode region of the first barcode molecule of the second multimeric barcoding reagent and the second barcoded target nucleic acid molecule comprises the nucleic acid sequence of the barcode region of the second barcode molecule of the second multimeric barcoding reagent.

The invention provides a method of preparing a nucleic acid sample for sequencing, wherein the sample comprises a cell, and wherein the method comprises the steps of: (a) contacting the sample with a multimeric barcoding reagent, wherein the multimeric barcoding reagent comprises first and second barcoded oligonucleotides linked together and a cell-binding moiety, wherein the barcoded oligonucleotides each comprise a barcode region, and wherein the cell-binding moiety of the multimeric barcoding reagent binds to the cell membrane of the cell and the first and second barcoded oligonucleotides of the multimeric barcoding reagent are internalized into the cell; and (b) annealing or ligating the first and second barcoded oligonucleotides of the multimeric barcoding reagent to first and second sub-sequences of a target nucleic acid of the cell to produce first and second barcoded target nucleic acid molecules.

The invention provides a method of preparing a nucleic acid sample for sequencing, wherein the sample comprises at least two cells, and wherein the method comprises the steps of: (a) contacting the sample with a library comprising at least two multimeric barcoding reagents, wherein each multimeric barcoding reagent comprises first and second barcoded oligonucleotides linked together and a cell-binding moiety, wherein the barcoded oligonucleotides each comprise a barcode region and wherein the barcode regions of the first and second barcoded oligonucleotides of a first multimeric barcoding reagent of the library are different to the barcode regions of the first and second barcoded oligonucleotides of a second multimeric barcoding reagent of the library, wherein the cell-binding moiety of a first multimeric barcoding reagent from the library binds to the cell membrane of a first cell of the sample and the first and second barcoded oligonucleotides of the first multimeric barcoding reagent are internalized into the first cell, and wherein the cell-binding moiety of a second multimeric barcoding reagent from the library binds to the cell membrane of a second cell of the sample and the first and second barcoded oligonucleotides of the second multimeric barcoding reagent are internalized into the second cell; and (b) annealing or ligating the first and second barcoded oligonucleotides of the first multimeric barcoding reagent to first and second sub-sequences of a target nucleic acid of the first cell to produce first and second barcoded target nucleic acid molecules, and annealing or ligating the first and second barcoded oligonucleotides from the second multimeric barcoding reagent to first and second sub-sequences of a target nucleic acid of the second cell to produce first and second barcoded target nucleic acid molecules.

In the methods, the cell binding and internalisation step may comprise an incubation period, wherein said incubation takes place for at least 5 seconds, at least 10 seconds, at least 30 seconds, at least 60 seconds, at least 2 minutes, at least 5 minutes, at least 10 minutes, at least 15 minutes, at least 30 minutes, at least 60 minutes, at least 2 hours, or at least 4 hours, optionally for 5 seconds to 4 hours, 10 seconds to 2 hours, 30 seconds to 60 minutes, 60 seconds to 30 minutes, 2 to 15 minutes or 5 to 10 minutes. Optionally, said incubation takes place at a temperature of at least 4 degrees Celsius, at least 12 degrees Celsius, at least 20 degrees Celsius, at least 30 degrees Celsius, at least 37 degrees Celsius, at least 40 degrees Celsius, at least 45 degrees Celsius, or at least 50 degrees Celsius, optionally at 4 to 50 degrees Celsius, 12 to 45 degrees Celsius, 20 to 40 degrees Celsius or 30 to 37 degrees Celsius.

The step of annealing or ligating (step (b)) may comprise: (i) annealing the first and second barcoded oligonucleotides of the first multimeric barcoding reagent to first and second sub-sequences of a target nucleic acid of the first cell, and annealing the first and second barcoded oligonucleotides of the second multimeric barcoding reagent to first and second sub-sequences of a target nucleic acid of the second cell; and (ii) extending the first and second barcoded oligonucleotides of the first multimeric barcoding reagent to produce first and second different barcoded target nucleic acid molecules and extending the first and second barcoded oligonucleotides of the second multimeric barcoding reagent to produce first and second different barcoded target nucleic acid molecules, wherein each of the barcoded target nucleic acid molecules comprises at least one nucleotide synthesised from the target nucleic acid as a template.

A cell-binding moiety may be attached to each of the barcoded oligonucleotides.

The multimeric barcoding reagents may each comprise: (i) first and second hybridization molecules linked together, wherein each of the hybridization molecules comprises a nucleic acid sequence comprising a hybridization region; and (ii) first and second barcoded oligonucleotides, wherein the first barcoded oligonucleotide is annealed to the hybridization region of the first hybridization molecule and wherein the second barcoded oligonucleotide is annealed to the hybridization region of the second hybridization molecule; optionally wherein the first multimeric barcoding reagent is internalized into the first cell and the second multimeric barcoding reagent is internalized into the second cell.

A cell-binding moiety may be attached to each of the hybridization molecules.

The multimeric barcoding reagents may each comprise: (i) first and second barcode molecules linked together, wherein each of the barcode molecules comprises a nucleic acid sequence comprising a barcode region; and (ii) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule; optionally wherein the first multimeric barcoding reagent is internalized into the first cell and the second multimeric barcoding reagent is internalized into the second cell.

A cell-binding moiety may be attached to each of the barcode molecules.

In the methods, the first multimeric barcoding reagent may be comprised within a first lipid carrier and the second multimeric barcoding reagent may be comprised within a second lipid carrier, optionally wherein in step (a) the first lipid carrier merges with the cell membrane of the first cell and the first and second barcoded oligonucleotides of the first multimeric barcoding reagent are internalized into the first cell, and the second lipid carrier merges with the cell membrane of the second cell and the first and second barcoded oligonucleotides of the first multimeric barcoding reagent are internalized into the second cell. Optionally, the barcoded oligonucleotides are released into the cell e.g. into the cytoplasm. The lipid carrier may be a liposome or a micelle. Alternatively, the lipid carrier may take any of the forms described herein.

The invention provides a method of preparing a nucleic acid sample for sequencing, wherein the sample comprises a cell, and wherein the method comprises the steps of: (a) contacting the sample with a multimeric barcoding reagent, wherein the multimeric barcoding reagent comprises: (i) first and second barcode molecules linked together, wherein each of the barcode molecules comprises a nucleic acid sequence comprising, optionally in the 5′ to 3′ direction, an adapter region and a barcode region, and (ii) first and second barcoded oligonucleotides, wherein the first barcoded oligonucleotide comprises a barcode region annealed to the barcode region of the first barcode molecule and wherein the second barcoded oligonucleotide comprises a barcode region annealed to the barcode region of the second barcode molecule; wherein the sample is further contacted with first and second adapter oligonucleotides for the multimeric barcoding reagent, wherein the first and second adapter oligonucleotides each comprise an adapter region, wherein a cell-binding moiety is attached to each of the adapter oligonucleotides, and wherein the cell-binding moieties of the first and second adapter oligonucleotides bind to the cell membrane of the cell and the first and second adapter oligonucleotides for the first multimeric barcoding reagent are internalized into the cell; (b) annealing or ligating the first and second adapter oligonucleotides for the multimeric barcoding reagent to sub-sequences of a target nucleic acid of the first cell; (c) annealing the adapter region of the first adapter oligonucleotide to the adapter region of the first barcode molecule, and annealing the adapter region of the second adapter oligonucleotide to the adapter region of the second barcode molecule; and (d) ligating the 3′ end of the first barcoded oligonucleotide to the 5′ end of the first adapter oligonucleotide to produce a first barcoded target nucleic acid molecule and ligating the 3′ end of the second barcoded oligonucleotide to the 5′ end of the second adapter oligonucleotide to produce a second barcoded target nucleic acid molecule.

In the methods, step (b) may comprise annealing the first and second adapter oligonucleotides to sub-sequences of a target nucleic acid of the cell, and wherein either: (i) step (d) comprises ligating the 3′ end of the first barcoded oligonucleotide to the 5′ end of the first adapter oligonucleotide to produce a first barcoded-adapter oligonucleotide and ligating the 3′ end of the second barcoded oligonucleotide to the 5′ end of the second adapter oligonucleotide to produce a second barcoded-adapter oligonucleotide, and extending the first and second barcoded-adapter oligonucleotides to produce first and second different barcoded target nucleic acid molecules each of which comprises at least one nucleotide synthesised from the target nucleic acid as a template, or (ii) before step (d), the method comprises extending the first and second adapter oligonucleotides to produce first and second different target nucleic acid molecules each of which comprises at least one nucleotide synthesised from the target nucleic acid as a template.

The invention provides a method of preparing a nucleic acid sample for sequencing, wherein the sample comprises at least two cells, and wherein the method comprises the steps of: (a) contacting the sample with a library comprising first and second multimeric barcoding reagents, wherein each multimeric barcoding reagent comprises: (i) first and second barcode molecules linked together, wherein each of the barcode molecules comprises a nucleic acid sequence comprising, optionally in the 5′ to 3′ direction, an adapter region and a barcode region, and (ii) first and second barcoded oligonucleotides, wherein the first barcoded oligonucleotide comprises a barcode region annealed to the barcode region of the first barcode molecule and wherein the second barcoded oligonucleotide comprises a barcode region annealed to the barcode region of the second barcode molecule, and wherein the barcode regions of the first and second barcoded oligonucleotides of the first multimeric barcoding reagent of the library are different to the barcode regions of the first and second barcoded oligonucleotides of the second multimeric barcoding reagent of the library; wherein the sample is further contacted with first and second adapter oligonucleotides for each of the multimeric barcoding reagents, wherein the first and second adapter oligonucleotides each comprise an adapter region, wherein a cell-binding moiety is attached to each of the adapter oligonucleotides, and wherein the cell-binding moieties of the first and second adapter oligonucleotides for the first multimeric barcoding reagent bind to the cell membrane of a first cell of the sample and the first and second adapter oligonucleotides for the first multimeric barcoding reagent are internalized into the first cell, and wherein the cell-binding moieties of the first and second adapter oligonucleotides for the second multimeric barcoding reagent bind to the cell membrane of a second cell of the sample and the first and second adapter oligonucleotides for the second multimeric barcoding reagent are internalized into the second cell; (b) annealing or ligating the first and second adapter oligonucleotides for the first multimeric barcoding reagent to sub-sequences of a target nucleic acid of the first cell, and annealing or ligating the first and second adapter oligonucleotides for the second multimeric barcoding reagent to sub-sequences of a target nucleic acid of the second cell; (c) for each of the multimeric barcoding reagents, annealing the adapter region of the first adapter oligonucleotide to the adapter region of the first barcode molecule, and annealing the adapter region of the second adapter oligonucleotide to the adapter region of the second barcode molecule; and (d) for each of the multimeric barcoding reagents, ligating the 3′ end of the first barcoded oligonucleotide to the 5′ end of the first adapter oligonucleotide to produce a first barcoded target nucleic acid molecule and ligating the 3′ end of the second barcoded oligonucleotide to the 5′ end of the second adapter oligonucleotide to produce a second barcoded target nucleic acid molecule.

In the methods, step (b) may comprise annealing the first and second adapter oligonucleotides for the first multimeric barcoding reagent to sub-sequences of a target nucleic acid of the first cell, and annealing the first and second adapter oligonucleotides for the second multimeric barcoding reagent to sub-sequences of a target nucleic acid of the second cell, and wherein either: (i) for each of the multimeric barcoding reagents, step (d) comprises ligating the 3′ end of the first barcoded oligonucleotide to the 5′ end of the first adapter oligonucleotide to produce a first barcoded-adapter oligonucleotide and ligating the 3′ end of the second barcoded oligonucleotide to the 5′ end of the second adapter oligonucleotide to produce a second barcoded-adapter oligonucleotide, and extending the first and second barcoded-adapter oligonucleotides to produce first and second different barcoded target nucleic acid molecules each of which comprises at least one nucleotide synthesised from the target nucleic acid as a template, or (ii) for each of the multimeric barcoding reagents, before step (d), the method comprises extending the first and second adapter oligonucleotides to produce first and second different target nucleic acid molecules each of which comprises at least one nucleotide synthesised from the target nucleic acid as a template.

The multimeric barcoding reagents may each comprise a cell-binding moiety, optionally wherein: (i) the cell-binding moiety of the first multimeric barcoding reagent binds to the cell membrane of the first cell of the sample and the multimeric barcoding reagent is internalized into the first cell and (ii) the cell-binding moiety of the second multimeric barcoding reagent binds to the cell membrane of the second cell of the sample and the second multimeric barcoding reagent is internalized into the second cell.

A cell-binding moiety may be attached to each of the barcode molecules. Additionally or alternatively, a cell-binding moiety may be attached to each of the barcoded oligonucleotides.

In the methods, the first and second adapter oligonucleotides for the first multimeric barcoding reagent may be comprised within a first lipid carrier and the first and second adapter oligonucleotides for the second multimeric barcoding reagent may be comprised within a second lipid carrier, optionally wherein in step (a) the first lipid carrier merges with the cell membrane of the first cell and the first and second adapter oligonucleotides for the first multimeric barcoding reagent are internalized into the first cell, and the second lipid carrier merges with the cell membrane of the second cell and the first and second adapter oligonucleotides for the second multimeric barcoding reagent are internalized into the second cell. Optionally, the adapter oligonucleotides are released into the cell e.g. into the cytoplasm.

The first lipid carrier may further comprise the first multimeric barcoding reagent and the second lipid carrier may further comprise the second multimeric barcoding reagent.

The lipid carrier may be a liposome or a micelle. Alternatively, the lipid carrier may take any of the forms described herein.

A cell-binding moiety may be attached to a multimeric barcoding reagent, adapter oligonucleotide, barcoded oligonucleotide, hybridization molecule or barcode molecule by a covalent linkage or by a non-covalent linkage.

A cell-binding moiety may be attached to each barcoded oligonucleotide, hybridization molecule, barcode molecule and/or adapter oligonucleotide by a linker molecule. Optionally, said linker may be a flexible linker. Optionally, said linker may be comprised of one or more units of ethylene glycol and/or poly(ethylene) glycol, such as hexa-ethylene glycol or penta-ethylene glycol. Optionally, said linker may be comprised of one or more ethyl groups, such as a C3 (three-carbon) spacer, C6, C12, or C18. Optionally, any other spacer may be used.

The cell-binding moiety (or moieties) may capable of initiating endocytosis on binding to a cell membrane.

The cell-binding moiety may comprise one or more moieties selected from: a peptide, a cell penetrating peptide, an aptamer, a DNA adptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a cationic lipid, a cationic polymer, poly(ethylene) glycol, spermine, a spermine derivatives or analogue, a poly-lysine, a poly-lysine derivative or analogue, polyethyleneimine, diethylaminoethyl (DEAE)-dextran, cholesterol, a sterol moiety, a cationic molecule, a hydrophobic molecule and an amphiphilic molecule.

The cell-binding moiety may interact with one or more specific molecule(s) on the cell surface or membrane (as in the case of e.g. an antibody, an antibody fragment and an aptamer). Alternatively or additionally, the cell-binding moiety may alter the overall charge and/or charge distribution of multimeric barcoding reagents (as in the case of e.g. a cationic polymer). Alternatively or additionally, the cell-binding moiety may alter the lipophilic/lipophobic and/or hydrophilic/hydrophobic character and/or balance of the multimeric barcoding reagents (as in the case of e.g. a lipid or cholesterol).

The cell-binding moiety may be a molecule that has a net positive charge in a solution comprising a cell and that enables binding of a multimeric barcoding reagent to the cell.

A multimeric barcoding reagent, adapter oligonucleotide, barcoded oligonucleotide, hybridization molecule or barcode molecule may comprise at least 2, at least 3, at least 5, at least 10, at least 20, at least 50, at least 100, at least 500, or at least 1000 cell binding moieties.

The invention provides a method of preparing a nucleic acid sample for sequencing, wherein the sample comprises at least two cells, (e.g. a cell from a cell line, or a cell originating from blood, or a cell originating from a tissue or organ sample, or a cell originating from a pre-implantation embryo generated by in vitro fertilisation), wherein the cell contains at least two fragments of a target nucleic acid (e.g. genomic DNA, and/or messenger RNA), and wherein the method comprises the steps of: (a) contacting the sample with a library comprising at least two multimeric barcoding reagents, wherein each multimeric barcoding reagent comprises first and second barcode regions linked together, wherein each barcode region comprises a nucleic acid sequence and wherein the first and second barcode regions of a first multimeric barcoding reagent are different to the first and second barcode regions of a second multimeric barcoding reagent of the library;

(b) transferring the first and second barcode regions of the first multimeric barcoding reagent from the library into a first cell of the sample and transferring the first and second barcode regions of the second multimeric barcoding reagent from the library into a second cell of the sample; and (c) appending barcode sequences to each of first and second sub-sequences of a target nucleic acid of the first cell to produce first and second barcoded target nucleic acid molecules for the first cell, wherein the first barcoded target nucleic acid molecule comprises the nucleic acid sequence of the first barcode region of the first multimeric barcoding reagent and the second barcoded target nucleic acid molecule comprises the nucleic acid sequence of the second barcode region of the first multimeric barcoding reagent, and appending barcode sequences to each of first and second sub-sequences of a target nucleic acid of the second cell to produce first and second barcoded target nucleic acid molecules from the second cell, wherein the first barcoded target nucleic acid molecule comprises the nucleic acid sequence of the first barcode region of the second multimeric barcoding reagent and the second barcoded target nucleic acid molecule comprises the nucleic acid sequence of the second barcode region of the second multimeric barcoding reagent.

The method of preparing a nucleic acid sample for sequencing, wherein the sample comprises at least two cells, may comprise the steps of: (a) contacting the sample with a library comprising at least two multimeric barcoding reagents, wherein each multimeric barcoding reagent comprises first and second barcoded oligonucleotides linked together, wherein the barcoded oligonucleotides each comprise a barcode region and wherein the barcode regions of the first and second barcoded oligonucleotides of a first multimeric barcoding reagent of the library are different to the barcode regions of the first and second barcoded oligonucleotides of a second multimeric barcoding reagent of the library; (b) transferring the first and second barcoded oligonucleotides of the first multimeric barcoding reagent from the library into a first cell of the sample and transferring the first and second barcoded oligonucleotides of the second multimeric barcoding reagent from the library into a second cell of the sample; and (c) annealing or ligating the first and second barcoded oligonucleotides of the first multimeric barcoding reagent to first and second sub-sequences of a target nucleic acid of the first cell to produce first and second barcoded target nucleic acid molecules, and annealing or ligating the first and second barcoded oligonucleotides from the second multimeric barcoding reagent to first and second sub-sequences of a target nucleic acid of the second cell to produce first and second barcoded target nucleic acid molecules.

In the methods, the step of annealing or ligating (step (c)) may comprise: (i) annealing the first and second barcoded oligonucleotides of the first multimeric barcoding reagent to first and second sub-sequences of a target nucleic acid of the first cell, and annealing the first and second barcoded oligonucleotides of the second multimeric barcoding reagent to first and second sub-sequences of a target nucleic acid of the second cell; and (ii) extending the first and second barcoded oligonucleotides of the first multimeric barcoding reagent to produce first and second different barcoded target nucleic acid molecules and extending the first and second barcoded oligonucleotides of the second multimeric barcoding reagent to produce first and second different barcoded target nucleic acid molecules, wherein each of the barcoded target nucleic acid molecules comprises at least one nucleotide synthesised from the target nucleic acid as a template.

In the methods, the multimeric barcoding reagents may each comprise: (i) first and second hybridization molecules linked together, wherein each of the hybridization molecules comprises a nucleic acid sequence comprising a hybridization region; and (ii) first and second barcoded oligonucleotides, wherein the first barcoded oligonucleotide is annealed to the hybridization region of the first hybridization molecule and wherein the second barcoded oligonucleotide is annealed to the hybridization region of the second hybridization molecule; optionally wherein step (b) comprises transferring the first multimeric barcoding reagent into the first cell and transferring the second multimeric barcoding reagent into the second cell.

In the methods, the multimeric barcoding reagents may each comprise: (i) first and second barcode molecules linked together, wherein each of the barcode molecules comprises a nucleic acid sequence comprising a barcode region; and (ii) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule; optionally wherein step (b) comprises transferring the first multimeric barcoding reagent into the first cell and transferring the second multimeric barcoding reagent into the second cell.

The invention provides a method of preparing a nucleic acid sample for sequencing, wherein the sample comprises at least two cells, and wherein the method comprises the steps of: (a) contacting the sample with a library comprising first and second multimeric barcoding reagents, wherein each multimeric barcoding reagent comprises: (i) first and second barcode molecules linked together, wherein each of the barcode molecules comprises a nucleic acid sequence comprising, optionally in the 5′ to 3′ direction, an adapter region and a barcode region, and (ii) first and second barcoded oligonucleotides, wherein the first barcoded oligonucleotide comprises a barcode region annealed to the barcode region of the first barcode molecule, wherein the second barcoded oligonucleotide comprises a barcode region annealed to the barcode region of the second barcode molecule, and wherein the barcode regions of the first and second barcoded oligonucleotides of the first multimeric barcoding reagent of the library are different to the barcode regions of the first and second barcoded oligonucleotides of the second multimeric barcoding reagent of the library; wherein the sample is further contacted with first and second adapter oligonucleotides for each of the multimeric barcoding reagents, wherein the first and second adapter oligonucleotides each comprise an adapter region; (b) transferring the first and second adapter oligonucleotides for the first multimeric barcoding reagent into the first cell and transferring the first and second adapter oligonucleotides for the second multimeric barcoding reagent into the second cell, optionally wherein the step further comprises transferring the first multimeric barcoding reagent into the first cell and transferring the second multimeric barcoding reagent into the second cell; (c) annealing or ligating the first and second adapter oligonucleotides for the first multimeric barcoding reagent to sub-sequences of a target nucleic acid of the first cell, and annealing or ligating the first and second adapter oligonucleotides for the second multimeric barcoding reagent to sub-sequences of a target nucleic acid of the second cell; (d) for each of the multimeric barcoding reagents, annealing the adapter region of the first adapter oligonucleotide to the adapter region of the first barcode molecule, and annealing the adapter region of the second adapter oligonucleotide to the adapter region of the second barcode molecule; and (e) for each of the multimeric barcoding reagents, ligating the 3′ end of the first barcoded oligonucleotide to the 5′ end of the first adapter oligonucleotide to produce a first barcoded target nucleic acid molecule and ligating the 3′ end of the second barcoded oligonucleotide to the 5′ end of the second adapter oligonucleotide to produce a second barcoded target nucleic acid molecule.

The cell sample may be contacted or bound with the multimeric barcoding reagent by, for example by mixing the cell sample with the multimeric barcoding reagent(s) in solution while the tube is stationary or with rotation of the tube. The cell sample may also be contacted or bound with the multimeric barcoding reagent by mixing the cell sample with the multimeric barcoding reagent in solution and allowing (and/or causing) them to settle (such as allowing them to settle by gravity and/or settling them by a centrifugation and/or pelleting process) at the bottom of a tube. Alternatively, the multimeric barcoding reagents could be settled at the bottom of a tube and the cell sample could be layered or settled on top of this multimeric barcoding reagent layer, or the cell sample could be settled at the bottom of a tube and the multimeric barcoding reagents could be layered or settled on top of this cell sample layer, or some combination of these settled layers may be used. The number of single cells within the sample used in this step may be at least 5, at least 10, at least 20, at least 25, at least 50, at least 75, at least 100, at least 250, at least 500, at least 10³, at least 10⁴, at least 10⁵, at least 10⁶, at least 10⁷, at least 10⁸, or at least 10⁹. The concentration of cells used in this step may be at least 10 cells/microliter or at least 50 cells/microliter or at least 100 cells/microliter or at least 500 cells/microliter or at least 10³ cells/microliter, at least 10⁴ cells/microliter, at least 10⁵ cells/microliter, at least 10⁶ cells/microliter, at least 10⁷ cells/microliter. The number of multimeric barcoding reagents used in this step may be at least 5, at least 10, at least 20, at least 25, at least 50, at least 75, at least 100, at least 250, at least 500, at least 10³, at least 10⁴, at least 10⁵, at least 10⁶, at least 10⁷, at least 10⁸, or at least 10⁹. The concentration of multimeric barcoding reagents used in this step may be at least 10 reagents/microliter, at least 100 reagents/microliter, at least 10³ reagents/microliter, at least 10⁴ reagents/microliter, at least 10⁵ reagents/microliter, at least 10⁶ reagents/microliter, at least 10⁷ reagents/microliter, at least 10⁸ reagents/microliter, or at least 10⁹ reagents/microliter. The ratio of single cells to multimeric barcoding reagents used in this step may be at least 0.1 or at least 0.5 or at least 1 or at least 2 or at least 5 or at least 10 or at least 100 or at least 1000. The settled layers of cells and multimeric barcoding reagents may be disrupted by pipette mixing to generate a more even solution of cells and reagents, while maintaining contacts between cells that are bound to a cell binding moiety on the multimeric barcoding reagent.

Optionally, during and/or following any step or process of contacting (or binding) a sample comprising cells with a library of multimeric barcoding reagents (and/or such as following any step of contacting, binding, mixing, centrifuging and/or settling cells and/or multimeric barcoding reagents, such as any step of settling cells onto multimeric barcoding reagents), and/or after any process of diluting cells and/or reagents (such as reagent-bound cells) into a larger volume and/or into a new buffer solution, such as a buffer solution or reaction for lysis and/or barcoding any individual multimeric barcoding reagent(s) may be bound to no (i.e zero) cells, or bound to a single cell, or bound to two cells, or bound to three cells, or bound to five cells, or bound to five or more cells; optionally, during and/or following any such step or process of contacting (or binding) and/or diluting, the ratio corresponding to the number of multimeric barcoding reagents that are each bound to two or more cells, divided by the number of multimeric barcoding reagents that are each bound to a single cell, may be less than 0.001%, less than 0.01%, less than 0.1%, less than 0.5%, less than 1%, less than 2%, less than 3%, less than 5%, less than 7%, less than 10%, less than 12%, less than 15%, less than 20%, less than 25%, less than 30%, less than 35%, less than 40%, less than 50%, or less than 60%; optionally, during and/or following any such step or process of contacting (or binding) and/or diluting, the ratio corresponding to the number of multimeric barcoding reagents that are each bound to two or more cells, divided by the number of multimeric barcoding reagents that are each bound to a single cell, may be approximately 0.001%, approximately 0.01%, approximately 0.1%, approximately 0.5%, approximately 1%, approximately 2%, approximately 3%, approximately 5%, approximately 7%, approximately 10%, approximately 12%, approximately 15%, approximately 20%, approximately 25%, approximately 30%, approximately 35%, approximately 40%, approximately 50%, or approximately 60%.

During and/or following any step or process of contacting (or binding) a sample comprising cells with a library of multimeric barcoding reagents (and/or such as following any step of contacting, binding, mixing, centrifuging and/or settling cells and/or multimeric barcoding reagents, such as any step of settling cells onto multimeric barcoding reagents; and/or after any process of diluting cells and/or reagents (such as reagent-bound cells) into a larger volume and/or into a new buffer solution, such as a buffer solution or reaction for lysis and/or barcoding), at least 1% of the cells, at least 2% of the cells, at least 5% of the cells, at least 10% of the cells, at least 15% of the cells, at least 20% of the cells, at least 25% of the cells, at least 30% of the cells, at least 35% of the cells, at least 40% of the cells, at least 50% of the cells, at least 60% of the cells, at least 70% of the cells, at least 75% of the cells, at least 80% of the cells, at least 90% of the cells, at least 95% of the cells, at least 97% of the cells, or at least 99% of the cells in the sample may be bound to a single reagent (or may be bound to one or more reagents); optionally, for each cell so bound to a single reagent (or so bound to one or more reagents), at least 2, at least 3, at least 5, at least 10, at least 25, at least 50, at least 100, at least 500, at least 1000, at least 5,000, at least 10,000, or at least 50,000 barcoded target nucleic acid molecules may be produced from the target nucleic acids of such cell.

During and/or following any step or process of contacting (or binding) a sample comprising cells with a library of multimeric barcoding reagents (and/or such as following any step of contacting, binding, mixing, centrifuging and/or settling cells and/or multimeric barcoding reagents, such as any step of settling cells onto multimeric barcoding reagents; and/or after any process of diluting cells and/or reagents (such as reagent-bound cells) into a larger volume and/or into a new buffer solution, such as a buffer solution or reaction for lysis and/or barcoding), approximately 1% of the cells, approximately 2% of the cells, approximately 5% of the cells, approximately 10% of the cells, approximately 15% of the cells, approximately 20% of the cells, approximately 25% of the cells, approximately 30% of the cells, approximately 35% of the cells, approximately 40% of the cells, approximately 50% of the cells, approximately 60% of the cells, approximately 70% of the cells, approximately 75% of the cells, approximately 80% of the cells, approximately 90% of the cells, approximately 95% of the cells, approximately 97% of the cells, or approximately 99% of the cells in the sample may be bound to a single reagent (or may be bound to one or more reagents); optionally, for each cell so bound to a single reagent (or so bound to one or more reagents), at least 2, at least 3, at least 5, at least 10, at least 25, at least 50, at least 100, at least 500, at least 1000, at least 5,000, at least 10,000, or at least 50,000 barcoded target nucleic acid molecules may be produced from the target nucleic acids of such cell.

During and/or following any step or process of contacting (or binding) a sample comprising cells with a library of multimeric barcoding reagents (and/or such as following any step of contacting, binding, mixing, centrifuging and/or settling cells and/or multimeric barcoding reagents, such as any step of settling cells onto multimeric barcoding reagents; and/or after any process of diluting cells and/or reagents (such as reagent-bound cells) into a larger volume and/or into a new buffer solution, such as a buffer solution or reaction for lysis and/or barcoding), approximately 1% of the cells, approximately 2% of the cells, approximately 5% of the cells, approximately 10% of the cells, approximately 15% of the cells, approximately 20% of the cells, approximately 25% of the cells, approximately 30% of the cells, approximately 35% of the cells, approximately 40% of the cells, approximately 50% of the cells, approximately 60% of the cells, approximately 70% of the cells, approximately 75% of the cells, approximately 80% of the cells, approximately 90% of the cells, approximately 95% of the cells, approximately 97% of the cells, or approximately 99% of the cells in the sample may be bound to a single reagent, wherein each such cell-bound single reagent is only bound to a single cell; optionally, for each cell so bound to a single reagent, at least 2, at least 3, at least 5, at least 10, at least 25, at least 50, at least 100, at least 500, at least 1000, at least 5,000, at least 10,000, or at least 50,000 barcoded target nucleic acid molecules may be produced from the target nucleic acids of such cell.

The cell sample may be contacted or bound with the multimeric barcoding reagent in a solution within a single 1.5 ml tube, or in a single 0.5 ml tube, or in a single 0.2 ml PCR tube, or in a 15 ml tube, or in a 50 ml tube, or in the wells of a V-bottom 96-well plate, or in the wells of a V-bottom 384-well plate, or in a flat bottom 96-well plate, or in a flat bottom 384-well plate, or in a round bottom 96-well plate, or in a round bottom 384-well plate, or on top of a uncoated glass microscope slide, or on a coated glass microscope slide or on a uncoated plastic microscope slide, or on a coated plastic microscope slide. Alternatively, any of the above pre-treated with a polymer coating solution on the interior surface of the vessel.

The cell sample may be contacted or bound with the multimeric barcoding reagent by mixing the cell sample with the multimeric barcoding reagent in solution with a reaction volume of at least 10 ul, or at least 20 ul, or at least 50 ul, or at least 100 ul, or at least 500 ul, or at least 1 ml, or at least 5 ml, or at least 10 ml, or at least 25 ml, or at least 50 ml. Within this total volume of cell sample and multimeric barcoding reagent the percentage of the volume which is cell sample could be at least 10%, or at least 20%, or at least 30%, or at least 40%, or at least 50%, or at least 60%, or at least 70%, or at least 80%, or at least 90%. Within this total volume of cell sample and multimeric barcoding reagent the percentage of the volume which is multimeric barcoding reagent could be at least 10%, or at least 20%, or at least 30%, or at least 40%, or at least 50%, or at least 60%, or at least 70%, or at least 80%, or at least 90%.

In the methods, the step of annealing or ligating (step (c)) may comprise annealing the first and second adapter oligonucleotides for the first multimeric barcoding reagent to sub-sequences of a target nucleic acid of the first cell, and annealing the first and second adapter oligonucleotides for the second multimeric barcoding reagent to sub-sequences of a target nucleic acid of the second cell, and wherein either:
(i) for each of the multimeric barcoding reagents, step (e) comprises ligating the 3′ end of the first barcoded oligonucleotide to the 5′ end of the first adapter oligonucleotide to produce a first barcoded-adapter oligonucleotide and ligating the 3′ end of the second barcoded oligonucleotide to the 5′ end of the second adapter oligonucleotide to produce a second barcoded-adapter oligonucleotide, and extending the first and second barcoded-adapter oligonucleotides to produce first and second different barcoded target nucleic acid molecules each of which comprises at least one nucleotide synthesised from the target nucleic acid as a template, or (ii) for each of the multimeric barcoding reagents, before step (e), the method comprises extending the first and second adapter oligonucleotides to produce first and second different target nucleic acid molecules each of which comprises at least one nucleotide synthesised from the target nucleic acid as a template.

In the methods, prior to the step of transferring (step (b)), the cell membrane of the cells may be permeabilised by contact with a chemical surfactant. Optionally, the barcoded oligonucleotides, adapter oligonucleotides and/or multimeric barcoding reagents are transferred into the cells through the permeabilised membrane.

The chemical surfactant may be a non-ionic surfactant. The chemical surfactant may be one or more of Triton X-100 (C₁₄H₂₂O(C₂H₄O)n(n=9-10)), Brij 35, Brij 58, Digitonin, IGEPAL CA-630, Saponin, TWEEN 20, TWEEN 40 and/or TWEEN 80.

The chemical surfactant may be in solution at a concentration of less than 1.0 micromolar, less than less than 5 micromolar, 10 micromolar, less than 25 micromolar, less than 50 micromolar, less than 100 micromolar, less than 200 micromolar, or less than 500 micromolar, less than 1.0 milimolar or less than 5.0 milimolar.

The cell(s) may be permeabilised by a mixture of two or more different chemical surfactants.

In the methods, after the step of permeabilising the cell membranes, the concentration of the chemical surfactant in the solution may be reduced by addition of a second solution to the sample comprising the cells and the chemical surfactant. Optionally, this second solution may not contain a chemical surfactant.

In the methods, after the step of permeabilising the cell membranes, the sample of cells may be pelleted by a centrifugation step, the supernatant (containing the chemical surfactant but not the cells) may be removed, and the pelleted cells may be resuspended in a second solution. Optionally, this second solution may not contain a chemical surfactant.

In the methods, prior to the step of transferring (step (b)), the cell membrane of the cells may be permeabilised by contact with a solvent or molecular solvent (capable of disturbing the lipid bilayer of the cell membrane). Optionally, the barcoded oligonucleotides, adapter oligonucleotides and/or multimeric barcoding reagents are transferred into the cells through the permeabilised membrane.

The solvent may be one or more of betaine, formamide, and/or dimethyl sulfoxide (DMSO)

The solvent may be used at a concentration of at least 1% by weight or by volume, at least 5% by weight or by volume, at least 10% by weight or by volume, at least 20% by weight or by volume, at least 30% by weight or by volume, at least 40% by weight or by volume, or at least 50% by weight or by volume.

In the methods, prior to the step of transferring (step (b)), the cell membrane of the cells may be permeabilised by a high-temperature thermal incubation step. Optionally, barcoded oligonucleotides, adapter oligonucleotides and/or multimeric barcoding reagents are transferred into the cells through the permeabilised membrane.

The thermal incubation step may be performed at a temperature of at least 37 degrees Celsius, at least 40 degrees Celsius, at least 45 degrees Celsius, at least 50 degrees Celsius, at least 55 degrees Celsius, at least 60 degrees Celsius, at least 65 degrees Celsius, at least 70 degrees Celsius, at least 75 degrees Celsius, at least 80 degrees Celsius, or at least 85 degrees Celsius.

The step of permeabilising the cell membranes may be performed for less than 5 seconds, less than 10 seconds, less than 30 seconds, less than 60 seconds, less than 2 minutes, less than 5 minutes, less than 10 minutes, less than 15 minutes, less than 30 minutes, less than 60 minutes, or less than 2 hours.

In the methods, the barcoded oligonucleotides, adapter oligonucleotides and/or multimeric barcoding reagents may be transferred into the cells by complexation with a transfection reagent or lipid carrier (followed by transfection, transfer, or release into the cells). This process may involve transfection, transfer or release of the reagents into the cell.

The transfection reagent may be a lipid transfection reagent e.g. a cationic lipid transfection reagent. Optionally, said cationic lipid transfection reagent comprises at least two alkyl chains. Optionally, said cationic lipid transfection reagent may be a commercially available cationic lipid transfection reagent such as Lipofectamine.

The barcoded oligonucleotides, adapter oligonucleotides and/or multimeric barcoding reagents may be transferred into the cells by complexation with a cationic polymer reagent (followed by transfection, transfer, or release into the cells). Optionally, said cationic polymer reagent may comprise a linear cationic polymer, such as spermine or poly-lysine. Optionally, said cationic polymer reagent may comprise a polyethyleneimine polymer. Optionally, said cationic polymer reagent may comprise a diethylaminoethyl (DEAE)-dextran polymer. Optionally, said cationic polymer reagent may comprise a branched cationic polymer.

The barcoded oligonucleotides, adapter oligonucleotides and/or multimeric barcoding reagents may be transferred into the cells by complexation with a dendrimer and/or an activated dendrimer (followed by transfection, transfer, or release into the cells). Optionally, said activated dendrimer is activated with one or more amino groups; optionally said amino groups are positively charged. Optionally, any such dendrimer and/or activated dendrimer comprises at least 2 generations, at least 3 generations, at least 5 generations, at least 10 generations, at least 20 generations, or at least 30 generations.

The barcoded oligonucleotides, adapter oligonucleotides and/or multimeric barcoding reagents may be transferred into the cells by complexation with a liposomal or micellar reagent (followed by transfection, transfer, or release into the cells). Optionally, the barcoded oligonucleotides, adapter oligonucleotides and/or multimeric barcoding reagents may be loaded into a preparation of liposomal or micellar reagents with a reagent loading step. Optionally, said liposomal or micellar reagents may comprise one or more amphiphiles. Optionally, said liposomal or micellar reagents may comprise one or more phospholipids. Optionally, said phospholipids may comprise one or more phosphatidylcholines. Optionally, said phospholipids may comprise one or more phophatidylethanolamine molecules. Optionally, said liposomal or micellar reagents may comprise copolymers. Optionally, said liposomal or micellar reagents may comprise block copolymers. Optionally, each liposomal or micellar reagent may on average be complexed with 1, or less than 1, or greater than 1, or any other number of multimeric barcoding reagent(s) within a preparation of such complexed multimeric barcoding reagent(s). Optionally, each liposomal or micellar reagent may on average be complexed with at least 2 barcoded oligonucleotides (and/or 2 adapter oligonucleotides).

The barcoded oligonucleotides, adapter oligonucleotides and/or multimeric barcoding reagents may be transferred into the cells by complexation within a solution of calcium chloride and phosphate to form a precipitate and then transfected into the cells.

The barcoded oligonucleotides, adapter oligonucleotides and/or multimeric barcoding reagents may be complexed to transfection reagents with a complexing incubation step. Optionally, this complexing incubation step may be at least 5 seconds, at least 10 seconds, at least 30 seconds, at least 60 seconds, at least 2 minutes, at least 5 minutes, at least 10 minutes, at least 30 minutes, at least 60 minutes, at least 2 hours in length, or at least 4 hours in length. Optionally, this complexing incubation step may take place at approximately 4 degrees Celsius, approximately 12 degrees Celsius, approximately 20 degrees Celsius, approximately 25 degrees Celsius, approximately 30 degrees Celsius, or approximately 37 degrees Celsius. Optionally, the complexed multimeric barcoding reagents may be further processed, and/or stored, prior to transfer into cells.

In the methods, after the barcoded oligonucleotides, adapter oligonucleotides and/or multimeric barcoding reagents are complexed to transfection reagents, a transfer incubation step may be performed. Optionally, this transfer incubation step may be at least 5 seconds, at least 10 seconds, at least 30 seconds, at least 60 seconds, at least 2 minutes, at least 5 minutes, at least minutes, at least 30 minutes, at least 60 minutes, at least 2 hours in length, or at least 4 hours 10 in length. Optionally, this transfer incubation step may take place at approximately 4 degrees Celsius, approximately 12 degrees Celsius, approximately 20 degrees Celsius, approximately 25 degrees Celsius, approximately 30 degrees Celsius, or approximately 37 degrees Celsius.

The barcoded oligonucleotides of the first multimeric barcoding reagent may be comprised within a first lipid carrier, and the barcoded oligonucleotides of the second multmeric barcoding reagent may be comprised within a second lipid carrier. Optionally, such barcoded oligonucleotides may be transferred into cells by a process involving merger of the liposome or micelle with the cell membrane. Optionally, this merger process may release the barcoded oligonucleotides into the cytoplasm of the cell. Optionally, the barcoded oligonucleotides may be loaded into a preparation of liposomal or micellar reagents with an oligonucleotide loading step. Optionally, said liposomes or micelles may comprise one or more amphiphiles. Optionally, said liposomes or micelles may comprise one or more phospholipids. Optionally, said phospholipids may comprise one or more phosphatidylcholines. Optionally, said phospholipids may comprise one or more phophatidylethanolamine molecules. Optionally, said liposomes or micelles may comprise copolymers. Optionally, said liposomes or micelles may comprise block copolymers. Optionally, each liposome or micelle may on average be complexed with, or loaded with, at least 2, at least 3, at least 5, at least 10, at least 50, at least 100, at least 500, at least 1000, at least 10,000, or at least 100,000 barcoded oligonucleotides, or any greater number of barcoded oligonucleotides.

In the methods, the barcoded oligonucleotides, adapter oligonucleotides and/or multimeric barcoding reagents may be transferred into the cells by a process comprising cell squeezing.

In the methods, the step of transferring may comprise mechanically deforming cells in the sample to produce transient membrane disruptions that enable the transfer of the barcoded oligonucleotides, adapter oligonucleotides and/or multimeric barcoding reagents into the cells. The sample may be contacted with a library of multimeric barcoding reagents (and/or adapter oligonucleotides for each multimeric barcoding reagent) before, during or after the step of mechanically deforming the cells.

Methods for cell squeezing are provided in Sharei et al, Cell Squeezing as a Robust, Microfluidic Intracellular Delivery Platform. J. Vis. Exp. (81, e50980, doi: 10.3791/50980 (2013), and Sharei et al, Proc Natl Acad Sci USA. 2013 Feb. 5; 110 (6): 2082-7).

In methods of cell squeezing intact cells may be shunted through a mechanical conduit (e.g. a microfluidic channel within a microfluidic circuit) that is smaller (i.e. smaller in diameter) than a cell, and wherein, as a cell transits through this conduit or channel, the cell becomes ‘squeezed’ (that is, it encounters a mechanical stress and/or deformation or shear stress) and is at least partially deformed. As a function of this process, the cell membrane becomes partially disturbed, and this may allow molecules (including barcoded oligonucleotides, adapter oligonucleotides and/or multimeric barcoding reagents) to transit from the solution surrounding the cell, into the cell itself. Cell squeezing thus comprises a mechanical, non-chemical, non-biological means of transferring reagents into cells.

The methods may comprise mixing a library of multimeric barcoding reagents with a sample of cells and passing the resulting mixture through a cell squeezing apparatus. This process allows multimeric barcoding reagents from the library thereof to enter one or more cells in the sample of cells. The resulting cells may then be further processed; for example, they may be incubated for a period of time e.g. to allow the barcoded oligonucleotides to anneal to cognate nucleic acids within the cells into which they have been transferred.

In the methods, the barcoded oligonucleotides, adapter oligonucleotides and/or multimeric barcoding reagents may be transferred into the cells by a process comprising electroporation (or electropermeabilisation).

The sample may be contacted with a library of multimeric barcoding reagents (and/or adapter oligonucleotides for each multimeric barcoding reagent) before, during or after the process of electroporation process.

The electroporation may use a square electroporation waveform. The electroporation may use an exponential electroporation waveform.

During the electroporation process the peak voltage gradient may be at least 1.0 kilovolts per centimetre, at least 2.0 kilovolts per centimetre, at least 5.0 kilovolts per centimetre, at least 10.0 kilovolts per centimetre, at least 15.0 kilovolts per centimetre, or at least 20.0 kilovolts per centimetre.

During the electroporation process the electroporation pulses may be at least 100 microseconds in duration, at least 500 microseconds in duration, at least 1.0 millisecond in duration, at least 2.0 milliseconds in duration, at least 3.0 milliseconds in duration, at least 5.0 milliseconds in duration, or at least 10.0 milliseconds in duration.

These below methods describe particular techniques for use with any of the above methods wherein multimeric barcoding reagents are transferred (or internalized) into cells by any method. These methods describe alternative embodiments, as well as subsequent experimental steps, that could potentially be applicable to any of the above protocols.

In the methods, following a step of transferring multimeric barcoding reagent(s) or adapter oligonucleotides into cells, the cells may be incubated for a period of time to allow the target regions of the multimeric barcoding reagent(s) to anneal to sub-sequences of a target nucleic acid within the cell. The incubation period may be at least 1 minute, or at least 5 minutes, or at least 15 minutes, or at least 30 minutes, or at least 60 minutes. The incubation may take place within a solution containing a nucleic acid denaturant, such as DMSO or betaine. The incubation may take place at a temperature of at least 37 degrees Celsius, at least 45 degrees Celsius, at least 50 degrees Celsius, at least 55 degrees Celsius, at least 60 degrees Celsius, at least 65 degrees Celsius, or at least 70 degrees Celsius.

In the methods, following a step of introducing barcoded oligonucleotides and/or multimeric barcoding reagent(s) into cells, a reagent-division step may be performed in which multimeric barcoding reagents divide into two or more independently diffusible components thereof. Optionally, in embodiments wherein a multimeric barcoding reagent comprises barcoded oligonucleotides annealed to barcode molecules, this reagent-division step may comprise a step of denaturing one or more barcoded oligonucleotides from the barcode molecules to which they are annealed, such that said barcoded oligonucleotides are able to diffuse independently within the cell(s) into which they have been transferred. Optionally, such a denaturing step may be performed with a high-temperature incubation, wherein the barcoded oligonucleotides are denatured at a temperature of at least 37 degrees Celsius, at least 45 degrees Celsius, at least 50 degrees Celsius, at least 55 degrees Celsius, at least 60 degrees Celsius, at least 65 degrees Celsius, or at least 70 degrees Celsius. Optionally, this denaturation step takes place within a solution containing a nucleic acid denaturant, such as DMSO or betaine. Optionally, this denaturation step may take place prior to an incubation step as described above; or optionally this denaturation step may take place within the same step as an incubation step.

In the methods, following the transfer of barcoded oligonucleotides, adapter oligonucleotides and/or multimeric barcoding reagents into cells, and optionally following an incubation step, the cells may be contacted by a solution of oligonucleotides complementary to all or part of one or more target regions of the barcoded oligonucleotides within multimeric barcoding reagents.

In the methods, following introduction of the barcoded oligonucleotides, adapter oligonucleotides and/or multimeric barcoding reagents into the cell, and optionally following an incubation step, the cell(s) may be isolated from a reaction mixture by centrifugation.

In the methods, following the transfer of the barcoded oligonucleotides, adapter oligonucleotides and/or multimeric barcoding reagents into the cell, and optionally following an incubation step, the barcoded oligonucleotides and/or barcoded target nucleic acid molecules and/or multimeric barcoding reagent(s) may be isolated from the cell.

The multimeric barcoding reagents and/or barcoded oligonucleotides may comprise one or more biotin moieties.

In the methods, following the transfer of barcoded oligonucleotides, adapter oligonucleotides and/or multimeric barcoding reagents into the cell, and optionally following an incubation step, the barcoded oligonucleotides and/or barcoded target nucleic acid molecules and/or multimeric barcoding reagent(s) may be isolated by a process of: (a) dissolving and/or permeabilising the cell membranes, optionally using a chemical surfactant, by using a (molecular) solvent, or by incubation at high temperature; (b) contacting the resulting mixture with a solid support, optionally wherein the solid support comprises streptavidin moieties; and (c) capturing the barcoded oligonucleotides and/or barcoded target nucleic acid molecules and/or multimeric barcoding reagent(s) on the solid support, optionally through streptavidin-biotin interaction.

The solid support may be one or more magnetic beads, optionally wherein the one or more magnetic beads comprise streptavidin molecules on their surface. The magnetic bead(s) may isolated from a reaction mixture with a magnet.

In the methods, any step(s) of permeabilising cells and/or transferring multimeric barcoding reagents into cells and/or incubating cells may take place in a hypotonic solution. In the methods, any step(s) of permeabilising cells and/or transferring multimeric barcoding reagents into cells and/or incubating cells may take place in a hypertonic solution.

In the methods, a library of multimeric barcoding reagents may be provided in the same solution as a chemical surfactant, and/or in the same solution as a molecular solvent, and/or in the same solution as a denaturant.

The invention provides a method of preparing a nucleic acid sample for sequencing, wherein the sample comprises at least two cells, and wherein the method comprises the steps of: (a) contacting the sample with a library comprising at least two multimeric barcoding reagents, wherein each multimeric barcoding reagent comprises first and second barcode regions linked together, wherein each barcode region comprises a nucleic acid sequence and wherein the first and second barcode regions of a first multimeric barcoding reagent are different to the first and second barcode regions of a second multimeric barcoding reagent of the library; (b) lysing the cells or permeabilizing the cell membranes of the cells; and (c) appending barcode sequences to each of first and second sub-sequences of a target nucleic acid of the first cell to produce first and second barcoded target nucleic acid molecules for the first cell, wherein the first barcoded target nucleic acid molecule comprises the nucleic acid sequence of the first barcode region of the first multimeric barcoding reagent and the second barcoded target nucleic acid molecule comprises the nucleic acid sequence of the second barcode region of the first multimeric barcoding reagent, and appending barcode sequences to each of first and second sub-sequences of a target nucleic acid of the second cell to produce first and second barcoded target nucleic acid molecules for the second cell, wherein the first barcoded target nucleic acid molecule comprises the nucleic acid sequence of the first barcode region of the second multimeric barcoding reagent and the second barcoded target nucleic acid molecule comprises the nucleic acid sequence of the second barcode region of the second multimeric barcoding reagent.

The invention provides a method of preparing a nucleic acid sample for sequencing, wherein the sample comprises at least two cells, and wherein the method comprises (in order) the steps of:
(a) contacting the sample with a library comprising at least two multimeric barcoding reagents, wherein each multimeric barcoding reagent comprises first and second barcoded oligonucleotides linked together, wherein the barcoded oligonucleotides each comprise a barcode region and wherein the barcode regions of the first and second barcoded oligonucleotides of a first multimeric barcoding reagent of the library are different to the barcode regions of the first and second barcoded oligonucleotides of a second multimeric barcoding reagent of the library;
(b) lysing the cells or permeabilizing the cell membranes of the cells; and
(c) annealing or ligating the first and second barcoded oligonucleotides of the first multimeric barcoding reagent to first and second sub-sequences of a target nucleic acid of the first cell to produce first and second barcoded target nucleic acid molecules, and annealing or ligating the first and second barcoded oligonucleotides from the second multimeric barcoding reagent to first and second sub-sequences of a target nucleic acid of the second cell to produce first and second barcoded target nucleic acid molecules.

The method may comprise (in order) the steps of: (a) contacting the sample with a library comprising at least two multimeric barcoding reagents, wherein each multimeric barcoding reagent comprises first and second barcoded oligonucleotides linked together, wherein the barcoded oligonucleotides each comprise a barcode region and wherein the barcode regions of the first and second barcoded oligonucleotides of a first multimeric barcoding reagent of the library are different to the barcode regions of the first and second barcoded oligonucleotides of a second multimeric barcoding reagent of the library; (b) lysing the cells or permeabilizing the cell membranes of the cells; and (c) annealing or ligating the first and second barcoded oligonucleotides of the first multimeric barcoding reagent to first and second sub-sequences of a target nucleic acid of the first cell to produce first and second barcoded target nucleic acid molecules, and annealing or ligating the first and second barcoded oligonucleotides from the second multimeric barcoding reagent to first and second sub-sequences of a target nucleic acid of the second cell to produce first and second barcoded target nucleic acid molecules.

The invention provides a method of preparing a nucleic acid sample for sequencing, wherein the sample comprises at least 10 cells, and wherein the method comprises in order the steps of: (a) contacting the sample with a library comprising at least two multimeric barcoding reagents, wherein each multimeric barcoding reagent comprises first and second barcoded oligonucleotides linked together and a cell-binding moiety, wherein the barcoded oligonucleotides each comprise a barcode region and wherein the barcode regions of the first and second barcoded oligonucleotides of a first multimeric barcoding reagent of the library are different to the barcode regions of the first and second barcoded oligonucleotides of a second multimeric barcoding reagent of the library, wherein the cell-binding moiety of the first multimeric barcoding reagent binds to the cell membrane of a first cell prior to step (b), and wherein the cell-binding moiety of the second multimeric barcoding reagent binds to the cell membrane of a second cell prior to step (b); (b) lysing the cells or permeabilizing the cell membranes of the cells; and (c) annealing or ligating the first and second barcoded oligonucleotides of the first multimeric barcoding reagent to first and second sub-sequences of a target nucleic acid of the first cell to produce first and second barcoded target nucleic acid molecules, and annealing or ligating the first and second barcoded oligonucleotides from the second multimeric barcoding reagent to first and second sub-sequences of a target nucleic acid of the second cell to produce first and second barcoded target nucleic acid molecules. Preferably, the cells are comprised within a single contiguous aqueous volume during steps (a), (b) and/or (c).

In the methods, the step of annealing or ligating (step (c)) may comprise: (i) annealing the first and second barcoded oligonucleotides of the first multimeric barcoding reagent to first and second sub-sequences of a target nucleic acid of the first cell, and annealing the first and second barcoded oligonucleotides of the second multimeric barcoding reagent to first and second sub-sequences of a target nucleic acid of the second cell; and (ii) extending the first and second barcoded oligonucleotides of the first multimeric barcoding reagent to produce first and second different barcoded target nucleic acid molecules and extending the first and second barcoded oligonucleotides of the second multimeric barcoding reagent to produce first and second different barcoded target nucleic acid molecules, wherein each of the barcoded target nucleic acid molecules comprises at least one nucleotide synthesised from the target nucleic acid as a template.

In the methods, the multimeric barcoding reagents may each comprise: (i) first and second hybridization molecules linked together, wherein each of the hybridization molecules comprises a nucleic acid sequence comprising a hybridization region; and (ii) first and second barcoded oligonucleotides, wherein the first barcoded oligonucleotide is annealed to the hybridization region of the first hybridization molecule and wherein the second barcoded oligonucleotide is annealed to the hybridization region of the second hybridization molecule.

The multimeric barcoding reagents may each comprise: (i) first and second barcode molecules linked together, wherein each of the barcode molecules comprises a nucleic acid sequence comprising a barcode region; and (ii) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule.

The invention provides a method of preparing a nucleic acid sample for sequencing, wherein the sample comprises at least two cells, and wherein the method comprises the steps of: (a) contacting the sample with a library comprising first and second multimeric barcoding reagents, wherein each multimeric barcoding reagent comprises: (i) first and second barcode molecules linked together, wherein each of the barcode molecules comprises a nucleic acid sequence comprising, optionally in the 5′ to 3′ direction, an adapter region and a barcode region, and (ii) first and second barcoded oligonucleotides, wherein the first barcoded oligonucleotide comprises a barcode region annealed to the barcode region of the first barcode molecule and wherein the second barcoded oligonucleotide comprises a barcode region annealed to the barcode region of the second barcode molecule, and wherein the barcode regions of the first and second barcoded oligonucleotides of the first multimeric barcoding reagent are different to the barcode regions of the first and second barcoded oligonucleotides of the second multimeric barcoding reagent; wherein the sample is further contacted with first and second adapter oligonucleotides for each of the multimeric barcoding reagents, wherein the first and second adapter oligonucleotides each comprise an adapter region; (b) lysing the cells or permeabilizing the cell membranes of the cells; (c) annealing or ligating the first and second adapter oligonucleotides for the first multimeric barcoding reagent to sub-sequences of a target nucleic acid of the first cell, and annealing or ligating the first and second adapter oligonucleotides for the second multimeric barcoding reagent to sub-sequences of a target nucleic acid of the second cell; (d) for each of the multimeric barcoding reagents, annealing the adapter region of the first adapter oligonucleotide to the adapter region of the first barcode molecule, and annealing the adapter region of the second adapter oligonucleotide to the adapter region of the second barcode molecule; and (e) for each of the multimeric barcoding reagents, ligating the 3′ end of the first barcoded oligonucleotide to the 5′ end of the first adapter oligonucleotide to produce a first barcoded target nucleic acid molecule and ligating the 3′ end of the second barcoded oligonucleotide to the 5′ end of the second adapter oligonucleotide to produce a second barcoded target nucleic acid molecule.

The cell sample may be contacted or bound with the multimeric barcoding reagent by, for example mixing the cell sample with the multimeric barcoding reagent in solution while the tube is stationary or with rotation of the tube. The cell sample may also be contacted or bound with the multimeric barcoding reagent by mixing the cell sample with the multimeric barcoding reagent in solution and allowing them to settle at the bottom of a tube. Alternatively, the multimeric barcoding reagents could be settled at the bottom of a tube and the cell sample could be layered or settled on top of this multimeric barcoding reagent layer, or the cell sample could be settled at the bottom of a tube and the multimeric barcoding reagents could be layered or settled on top of this cell sample layer, or some combination of these settled layers may be used. The number of single cells within the sample used in this step may be at least 5, at least 10, at least 20, at least 25, at least 50, at least 75, at least 100, at least 250, at least 500, at least 10³, at least 10⁴, at least 10⁵, at least 10⁶, at least 10⁷, at least 10⁸, or at least 10⁹. The concentration of cells used in this step may be at least 10 cells/microliter or at least 50 cells/microliter or at least 100 cells/microliter or at least 500 cells/microliter or at least 10³ cells/microliter, at least 10⁴ cells/microliter, at least 10⁵ cells/microliter, at least 10⁶ cells/microliter, at least 10⁷ cells/microliter. The number of multimeric barcoding reagents used in this step may be at least 5, at least 10, at least 20, at least 25, at least 50, at least 75, at least 100, at least 250, at least 500, at least 10³, at least 10⁴, at least 10⁵, at least 10⁶, at least 10⁷, at least 10⁸, or at least 10⁹. The concentration of multimeric barcoding reagents used in this step may be at least 10 reagents/microliter, at least 100 reagents/microliter, at least 10³ reagents/microliter, at least 10⁴ reagents/microliter, at least 10⁵ reagents/microliter, at least 10⁶ reagents/microliter, at least 10⁷ reagents/microliter, at least 10⁸ reagents/microliter, or at least 10⁹ reagents/microliter. The ratio of single cells to multimeric barcoding reagents used in this step may be at least 0.1 or at least 0.5 or at least 1 or at least 2 or at least 5 or at least 10 or at least 100 or at least 1000. The settled layers of cells and multimeric barcoding reagents may be disrupted by pipette mixing to generate a more even solution of cells and reagents, while maintaining contacts between cells that are bound to a cell binding moiety on the multimeric barcoding reagent.

In the methods, the step of annealing or ligating (step (c)) may comprise annealing the first and second adapter oligonucleotides for the first multimeric barcoding reagent to sub-sequences of a target nucleic acid of the first cell, and annealing the first and second adapter oligonucleotides for the second multimeric barcoding reagent to sub-sequences of a target nucleic acid of the second cell, and wherein either: (i) for each of the multimeric barcoding reagents, step (e) comprises ligating the 3′ end of the first barcoded oligonucleotide to the 5′ end of the first adapter oligonucleotide to produce a first barcoded-adapter oligonucleotide and ligating the 3′ end of the second barcoded oligonucleotide to the 5′ end of the second adapter oligonucleotide to produce a second barcoded-adapter oligonucleotide, and extending the first and second barcoded-adapter oligonucleotides to produce first and second different barcoded target nucleic acid molecules each of which comprises at least one nucleotide synthesised from the target nucleic acid as a template, or (ii) for each of the multimeric barcoding reagents, before step (e), the method comprises extending the first and second adapter oligonucleotides to produce first and second different target nucleic acid molecules each of which comprises at least one nucleotide synthesised from the target nucleic acid as a template.

In the methods, following the step of lysing or permeabilising (step (b)), target nucleic acids from each cell within the sample may be able to diffuse out of the cell (i.e. out of the cytoplasmic space or cell volume). Optionally, the multimeric barcoding reagents are not able to enter the cell. Optionally, following step (b), the cell membrane is substantially or totally dissolved. Optionally, following step (b), the cell membrane remains partially intact but wherein messenger RNA molecules and/or other nucleic acid molecules are able to diffuse out of the cell (i.e. out of the cytoplasmic space or cell volume) through pores and/or other structural discontinuities within the cell membrane.

In the methods, step (b) may be performed by increasing the temperature of the sample. Optionally, a high temperature incubation step may be performed, for example the high temperature incubation step may be performed at a temperature of at least 37 degrees Celsius, at least 40 degrees Celsius, at least 50 degrees Celsius, at least 60 degrees Celsius, at least 70 degrees Celsius, at least 80 degrees Celsius, at least 90 degrees Celsius, or at least 95 degrees Celsius. The incubation step may be at least 1 second long, at least 5 seconds long, at least 10 seconds long, at least 30 seconds long, at least 1 minute long, at least 5 minutes long, at least 10 minutes long, at least 30 minutes long, at least 60 minutes long, or at least 3 hours long.

In the methods, step (b) may be performed in the presence of a chemical surfactant. The chemical surfactant may be a non-ionic surfactant. The chemical surfactant may be one or more of Triton X-100 (C₁₄H₂₂O(C₂H₄O) n (n=9-10)), Brij 35, Brij 58, Digitonin, IGEPAL CA-630, Saponin, Sodium dodecyl sulfate, Sodium n-octadecyl sulfate, Trimethyloctadecylammonium bromide, TWEEN 20, TWEEN 40 and/or TWEEN 80.

In the methods, step (b) may be performed in the presence of a solvent or molecular solvent (capable of disturbing the lipid bilayer of the cell membrane). The solvent may be one or more of betaine, formamide, and/or dimethyl sulfoxide (DMSO).

In the methods, any step(s) may take place under hypotonic or hypertonic conditions. Optionally, step (b) may be performed under hypotonic or hypertonic conditions.

In any of the methods described herein, the sample of or cells may be digested with a proteinase digestion step, such as a digestion with a Proteinase K enzyme. Optionally, this proteinase digestion step may be at least 10 seconds long, at least 30 seconds long, at least 60 seconds long, at least 5 minutes long, at least 10 minutes long, at least 30 minutes long, at least 60 minutes long, at least 3 hours long, at least 6 hours long, at least 12 hours long, or at least 24 hours long. This step may be performed before any permeabilisation step, after any permeabilisation step, before any step of appending coupling sequences, after any step of appending couplings sequences, before any step of appending barcode sequences (e.g. before step (c)), after any step of appending barcode sequences (e.g. after step (c)), whilst appending barcode sequences, or any combination thereof. For example, prior to contacting a sample comprising cells with a library of two or more multimeric barcoding reagents, the sample comprising cells may be crosslinked, and then partially digested with a Proteinase K digestion step. Optionally, any Proteinase K digestion step may optionally have the effect of partially and/or fully cleaving and/or digesting proteins and/or polypeptides and/or protein complexes and/or any type of macromolecular complex comprising one or more proteins in addition to one or more non-protein molecules, such as any one or more nucleosomal structures and/or nucleosomal and/or histone and/or chromatin proteins/complexes that are associated with and/or bound to one or more DNA molecules (e.g. genomic DNA molecules and/or mitochondrial DNA molecules).

In the methods, the multimeric barcoding reagents and/or adapter oligonucleotides may each comprise a cell-binding moiety, optionally wherein the cell-binding moiety binds each multimeric barcoding reagent and/or adapter oligonucleotide to the cell membrane of a cell prior to step (b). Optionally, each of the barcoded oligonucleotides, multimeric hybridization molecules and/or multimeric barcode molecules comprise a cell-binding moiety. The cell-binding moiety of each barcoded oligonucleotide, multimeric hybridization molecule and/or multimeric barcode molecule may bind to the cell membrane of a cell prior to step (b).

In combination with, or as an alternative to the cell binding moiety existing within or on the construct of the multimeric barcoding reagents, the binding moiety may be localised to the target cells in a ‘cell priming’ reaction. In such a cell priming reaction, single cell suspensions may be incubated in a solution containing molecules which enable tethering or binding of the multimeric barcoding reagents to the cell membranes. Such molecules may be polymeric cation molecules, such as those described previously, such as poly-L-lysine. Alternatively, cell membrane binding conjugated oligonucleotides, such as those described previously, may be deployed to bind the membranes in such a cell priming reaction.

Priming may be performed for a period of time of; at least 30 seconds, at least 1 minute, at least 2 minutes, at least 5 minutes, at least 10 minutes, at least 20 minutes or at least 30 minutes. The localised concentrations of cell binding oligonucleotides used in cell priming reactions may be at least 1 nM, at least 5 nM, at least 10 nM, at least 20 nM, at least 50 nM, at least 100 nM, at least 200 nM, at least 500 nM, at least 1000 nM, at least 2000 nM, or at least 5000 nM. Concentrations of cationic molecules used in solution used during the priming protocol may be at least 0.0001%, at least 0.001%, at least 0.01%, at least 0.1%, at least 1% or at least 10%.

Following the priming reaction, cell and multimeric barcoding reagent binding may be performed. Cell binding may be performed in any of the ways described previously. In such binding reactions the interactions may differ to those observed when the cell binding moiety is localised to the multimeric barcoding reagents. Such reactions may rely on oligonucleotide hybridization between the multimeric hybridization molecule (e.g. the multimeric barcode molecule) and the cell binding moiety oligonucleotide displaying on the cell membrane. Alternatively, with polymeric cation molecules, binding may be more comparable to that expected if the polymeric cation molecules were adhered to the multimeric barcoding reagents rather than the cells.

In the methods, the step of annealing barcoded oligonucleotides to target nucleic acids may comprise an incubation step, wherein the sample is incubated for a period of time to allow the target regions of the barcoded oligonucleotides to anneal to target nucleic acids. Optionally, this incubation period is at least 1 minute, or at least 5 minutes, or at least 15 minutes, or at least 30 minutes, or at least 60 minutes. Optionally, this incubation takes place within a solution containing a nucleic acid denaturant or a nucleic acid hybridization stabilizing agent, such as DMSO or betaine or urea or formamide or trehalose or tetramethylammonium chloride, or in a solution containing a nucleic acid protector such as guanidine isothiocyanate. Optionally, this incubation takes place within a solution containing a nuclease inhibitor such as a recombinant ribonuclease inhibitor. Optionally, this incubation takes place within a solution containing a reducing agent such as dithiothreitol or 2-mercaptoethanol. Optionally, this incubation takes place at a temperature of at least 37 degrees Celsius, at least 45 degrees Celsius, at least 50 degrees Celsius, at least 55 degrees Celsius, at least 60 degrees Celsius, at least 65 degrees Celsius, or at least 70 degrees Celsius.

In the methods, during or prior to step (c), a reagent-division step may be performed in which multimeric barcoding reagents are divided into two or more independently diffusible components thereof. Optionally, wherein a multimeric barcoding reagent comprises barcoded oligonucleotides annealed to barcode molecules, this reagent-division step comprises a step of denaturing one or more barcoded oligonucleotides from the barcode molecules to which they are annealed, such that said barcoded oligonucleotides are able to diffuse independently within solution. Optionally, such a denaturing step may be performed with a high-temperature incubation, wherein the barcoded oligonucleotides are denatured at a temperature of at least 37 degrees Celsius, at least 45 degrees Celsius, at least 50 degrees Celsius, at least 55 degrees Celsius, at least 60 degrees Celsius, at least 65 degrees Celsius, or at least 70 degrees Celsius. Optionally, this denaturation step takes place within a solution containing a nucleic acid denaturant, such as DMSO or betaine or urea or formamide or trehalose or tetramethylammonium chloride, or in a solution containing a nucleic acid protector such as guanidine isothiocyanate. Optionally, this reagent-division step and/or denaturation step may take place prior to an annealing step as described above; or optionally this reagent-division step and/or denaturation step may take place during the annealing step. Additionally, this reagent-division step and/or denaturation step may take place during the cell lysis step. For example, a single high-temperature thermal incubation step may have the effect of lysing cells through a thermal lysis process, and denaturing barcoded oligonucleotides from barcode molecules within multimeric barcoding reagents. Additionally, such a combined, high-temperature cell-lysis and reagent-division step may take place at the same temperature of and/or during the step of annealing the barcoded oligonucleotides to target nucleic acids.

The nucleic acid sample may have a concentration of cells for step (a) of less than 10 picomolar, less than 1 picomolar, less than 100 femtomolar, less than 10 femtomolar, less than 1 femtomolar, less than 100 attomolar, less than 10 attomolar, or less than 1 attomolar. Alternative higher or lower concentrations may also be used. Preferably, the cells will be at a concentration of less than 10 femtomolar.

The nucleic acid sample may have a concentration of cells for step (b) of less than 10 picomolar, less than 1 picomolar, less than 100 femtomolar, less than 10 femtomolar, less than 1 femtomolar, less than 100 attomolar, less than 10 attomolar, or less than 1 attomolar. Alternative higher or lower concentrations may also be used. Preferably, the cells will be at a concentration of less than 10 femtomolar.

The nucleic acid sample may have a concentration of cells for step (c) of less than 10 picomolar, less than 1 picomolar, less than 100 femtomolar, less than 10 femtomolar, less than 1 femtomolar, less than 100 attomolar, less than 10 attomolar, or less than 1 attomolar. Alternative higher or lower concentrations may also be used. Preferably, the cells will be at a concentration of less than 10 femtomolar.

In the methods, prior to step (b), the method may comprise diluting the nucleic acid sample. The step of diluting the sample may be performed after a step of binding cell-binding moieties (of adapter oligonucleotides, barcoded oligonucleotides and/or multimeric barcoding reagents) to cell membranes of cells in the sample. The nucleic acid sample may have a concentration of cells for step (a) and/or step (b) and/or step (c) of less than 10 picomolar, less than 1 picomolar, less than 100 femtomolar, less than 10 femtomolar, less than 1 femtomolar, less than 100 attomolar, less than 10 attomolar, or less than 1 attomolar. Alternative higher or lower concentrations may also be used. Preferably, the cells will be at a concentration of less than 10 femtomolar. Having a low concentration of cells in the nucleic acid sample during steps (b) and (c) may reduce the ‘cross-barcoding’ of two physically close cells by the same multimeric barcoding reagent.

In the methods, any of steps (a), (b) and/or (c) may be performed in a high-viscosity solution. Optionally, such a high-viscosity solution may be comprised of a poly(ethylene) glycol (PEG) solution, such as PEG 4000 or PEG 6000 or PEG 8000 or PEG 10,000 or PEG 20,000 or PEG 35,000 or PEG 40,000. Optionally, such a solution may comprise at least 5% poly(ethylene) glycol, at least 10% poly(ethylene) glycol, at least 20% poly(ethylene) glycol, at least 25% poly(ethylene) glycol, at least 30% poly(ethylene) glycol, at least 40% poly(ethylene) glycol, or at least 50% poly(ethylene) glycol by weight or by volume. Optionally, such a high-viscosity solution may be comprised of a polyvinylpyrrolidone (PVP) solution, such as PVP 10,000 or PVP 20,000 or PVP 35,000. Optionally, such a solution may comprise at least 5% PVP, at least 10% PVP, at least 20% PVP, at least 25% PVP, at least 30% PVP, at least 40% PVP, or at least 50% PVP by weight or by volume. Optionally, such a high-viscosity solution may be comprised of a dextran solution, such as dextran 5000. Optionally, such a solution may comprise at least 5% dextran, at least 10% dextran, at least 20% dextran, at least 25% dextran, at least 30% dextran, at least 40% dextran, or at least 50% dextran by weight or by volume. Optionally, such a high-viscosity solution may be comprised of a polyvinyl acetate (PVA) or a polyacryclic acid (PAA) solution, such as PVA 10,000 or PAA 8,000. Optionally, such a solution may comprise at least 5% PVA or PAA, at least 10% PVA or PAA, at least 20% PVA or PAA, at least 25% PVA or PAA, at least 30% PVA or PAA, at least 40% PVA or PAA, or at least 50% dextran by weight or by volume. Optionally, such a high-viscosity solution may be comprised of a chitosan solution such as chitosan 5000. Optionally, such a solution may comprise at least 5% chitosan, at least 10% chitosan, at least 20% chitosan, at least 25% chitosan, at least 30% chitosan, at least 40% chitosan, or at least 50% chitosan by weight or by volume. Optionally, such a solution may comprise a mixture of two or more different high-viscosity agents. Optionally, such a high-viscosity solution may comprise a solidified or semi-solidified gel or hydrogel, such as an agarose gel, a polyacrylamide gel, a crosslinked gel such as a crosslinked PEG-acrylate/PEG-thiol hydrogel, or a block-copolymer gel. Optionally, such a high-viscosity solution may comprise the solution employed during any step of cell lysis and/or cell permeabilisation. Optionally, such a high-viscosity solution may comprise the solution employed during any step of annealing barcoded oligonucleotides to target nucleic acids. Optionally, such a high-viscosity solution may have a dynamic viscosity of at least 1.0 centipoise, at least 1.1 centipoise, at least 1.2 centipoise, at least 1.5 centipoise, at least 2.0 centipoise, at least 5.0 centipoise, at least 10.0 centipoise, at least 20.0 centipoise, at least 50.0 centipoise, at least 100.0 centipoise, or at least 200.0 centipoise (e.g. at 25 degrees Celsius at standard sea-level pressure). Preferably, such a high-viscosity solution will have a dynamic viscosity of at least 2.0 centipoise. The use of a high-viscosity solution may slow the diffusion of the barcoded oligonucleotides and their target nucleic acids away from each other—i.e. when a multimeric barcoding reagent has been bound to the membrane of a single particular cell, and then the membrane is lysed or permeabilised, a high viscosity solution will have the effect of keeping the barcoded oligonucleotides and target nucleic acids from the cells in the vicinity of the original cell for a longer period of time-thus keeping the effective ‘concentration’ of both higher for a longer period of time (since they will occupy a smaller overall volume for a longer period of time). This slowed diffusion may also have the further effect of slowing the diffusion of target nucleic acids from one cell into a volume occupied by target nucleic acids from another cell.

Optionally, in any one or more steps of any of the methods (such as any step of appending coupling sequences and/or coupling molecules, any step of appending barcode sequences such as any step of appending and/or linking and/or connecting barcoded oligonucleotides (such as any step of appending/linking/connecting barcode sequences comprised within barcoded oligonucleotides), any step of permeabilising and/or lysing a sample (such as any step of permeabilising and/or lysing a sample comprising one or more cells, and/or any step of permeabilising and/or lysing a sample comprising one or more cells), the step(s) and/or method(s) may be performed in a solution containing any concentration of any one or more monovalent or divalent salt, such as any concentration of MgCl2 (such at least 0.5 mM MgCl2, at least 1.0 mM MgCl2, at least 1.5 mM MgCl2, at least 2.0 mM MgCl2, at least 2.5 mM MgCl2, at least 3.0 mM MgCl2, at least 3.5 mM MgCl2, at least 4.0 mM MgCl2, at least 4.5 mM MgCl2, at least 5.0 mM MgCl2, at least 6.0 mM MgCl2, at least 7.0 mM MgCl2, at least 8.0 mM MgCl2, at least 9.0 mM MgCl2, at least 10.0 mM MgCl2, at least 15.0 mM MgCl2, at least 20.0 mM MgCl2, or at least 25.0 mM MgCl2). Optionally, any such concentration may be any concentration of NaCl (such as at least 0.5 mM NaCl, at least 1.0 mM NaCl, at least 1.5 mM NaCl, at least 2.0 mM NaCl, at least 2.5 mM NaCl, at least 3.0 mM NaCl, at least 3.5 mM NaCl, at least 4.0 mM NaCl, at least 4.5 mM NaCl, at least 5.0 mM NaCl, at least 6.0 mM NaCl, at least 7.0 mM NaCl, at least 8.0 mM NaCl, at least 9.0 mM NaCl, at least 10.0 mM NaCl, at least 15.0 mM NaCl, at least 20.0 mM NaCl, at least 25.0 mM NaCl, at least 50.0 mM NaCl, at least 75.0 mM NaCl, at least 100.0 mM NaCl, at least 125.0 mM NaCl, at least 150.0 mM NaCl, at least 175.0 mM NaCl, at least 200.0 mM NaCl, at least 225.0 mM NaCl, at least 250.0 mM NaCl, at least 300.0 mM NaCl, at least 350.0 mM NaCl, at least 400.0 mM NaCl, at least 450.0 mM NaCl, at least 500.0 mM NaCl, at least 750.0 mM NaCl, or at least 1.0 M NaCl). Optionally, any such concentration may be any concentration of LiCl (such as at least 0.5 mM LiCl, at least 1.0 mM LiCl, at least 1.5 mM LiCl, at least 2.0 mM LiCl, at least 2.5 mM LiCl, at least 3.0 mM LiCl, at least 3.5 mM LiCl, at least 4.0 mM LiCl, at least 4.5 mM LiCl, at least 5.0 mM LiCl, at least 6.0 mM LiCl, at least 7.0 mM LiCl, at least 8.0 mM LiCl, at least 9.0 mM LiCl, at least 10.0 mM LiCl, at least 15.0 mM LiCl, at least 20.0 mM LiCl, at least 25.0 mM LiCl, at least 50.0 mM LiCl, at least 75.0 mM LiCl, at least 100.0 mM LiCl, at least 125.0 mM LiCl, at least 150.0 mM LiCl, at least 175.0 mM LiCl, at least 200.0 mM LiCl, at least 225.0 mM LiCl, at least 250.0 mM LiCl, at least 300.0 mM LiCl, at least 350.0 mM LiCl, at least 400.0 mM LiCl, at least 450.0 mM LiCl, at least 500.0 mM LiCl, at least 750.0 mM LiCl, or at least 1.0 M LiCl). Optionally, a solution comprising any other one or more other monovalent or divalent salt(s) may be employed in any such one or more steps of any of the method(s), at any concentration(s) as above, such as KCl, and/or potassium acetate, and/or magnesium acetate, and/or ammonium sulfate, and/or magnesium sulfate, and/or potassium sulfate. Optionally, any solution employed for any such above step may hold any pH (such as a pH at any temperature, such as pH at 25 degrees Celsius), such as at least pH 5.5, at least pH 6.0, at least pH 6.3, at least pH 6.5, at least pH 6.8, at least pH 7.0, at least pH 7.2, at least pH 7.5, at least pH 7.8, at least pH 7.9, at least pH 8.0, at least pH 8.3, at least pH 8.5, at least pH 8.8, at least pH 9.0, at least pH 9.3, at least pH 9.5, at least pH 9.8, or at least pH 10; and/or any such solution may comprise any buffer (e.g. for establishing and/or retaining such pH) such as TAPS, Tris (eg Tris-HCl and/or Tris-acetate and/or Tris-SO4), and/or bis-tris-propane, and/or otherwise, and/or other additives such as BSA (bovine serum albumin), DTT, glycerol, b-mercaptoethanol, and/or EDTA.

In the methods, after contacting a sample comprising cells with a library of at least 2 multimeric barcoding reagents, the barcoded oligonucleotides may be digested or partially digested with an exonuclease-digestion step. Optionally, this exonuclease-digestion step may be performed before, or may be performed after, a step of transferring multimeric barcoding reagents into cells. Optionally, this exonuclease-digestion step may be performed before, or may be performed after, a step of annealing barcoded oligonucleotides to target nucleic acids from cells. Optionally, this exonuclease-digestion step may be performed by E. coli Exonuclease I, or E. coli Lambda exonuclease.

In the methods, a sample comprising cells and/or a library of two or more multimeric barcoding reagents may be contacted with a solution of one or more blocking oligonucleotides, wherein said blocking oligonucleotides may be complementary to all or part of one or more barcoded oligonucleotides. Optionally, said blocking oligonucleotides may be complementary to all or part of the target region of one or more barcoded oligonucleotides.

In the methods, a sample comprising cells and/or a library of two or more multimeric barcoding reagents may be contacted with a solution of one or more blocking oligonucleotides, wherein the blocking oligonucleotides may be complementary to all or part of one or more target nucleic acids. Optionally, the blocking oligonucleotides may be complementary to one or more specific DNA or RNA sequences. Optionally, the blocking oligonucleotides may be complementary to one or more messenger RNA (mRNA) sequences. Optionally, the blocking oligonucleotides may be complementary to the poly(A) tail sequence of messenger RNA (mRNA) sequences. Optionally, the blocking oligonucleotides may comprise a poly(T) sequence of at least 2, at least 3, at least 5, at least 10, at least 20, at least 30, or at least 50 nucleotides that are complementary to the poly(A) tail sequence of messenger RNA (mRNA) sequences.

Optionally, any said blocking oligonucleotides may anneal to the respective sequences to which they are complementary or partially complementary. Optionally, the annealing temperature at which such blocking oligonucleotides hybridise to their respective complementary sequences may be lower than the temperature at which the target region of the barcoded oligonucleotides hybridise to the target region of their target cellular nucleic acids. Optionally, this blocking-oligonucleotide step may be performed before, or may be performed after, a step of contacting a sample of cells with a library of two or more multimeric barcoding reagents. Optionally, this blocking-oligonucleotide step may be performed before, or may be performed after, a step of transferring multimeric barcoding reagents into cells. Optionally, this blocking-oligonucleotide step may be performed before, or may be performed after, a step of binding multimeric barcoding reagents to the surface of cells, wherein said multimeric barcoding reagents comprise cell-binding moieties. Optionally, this blocking-oligonucleotide step may be performed before, or may be performed after, a step of lysing or permeabilising cells. Optionally, this blocking-oligonucleotide step may be performed before, or may be performed after, a step of annealing barcoded oligonucleotides to nucleic acids from cells. Optionally, this blocking-oligonucleotide step may be performed after a step of annealing barcoded oligonucleotides to nucleic acids from cells, wherein the blocking-oligonucleotide step comprises a process of lowering the temperature of the sample solution to a temperature at or below the temperature at which the blocking oligonucleotides anneal to their respective sequences. Optionally, this blocking oligonucleotide step may be performed upon a library of multimeric barcoding reagents, prior to contacting a sample of cells with said library. Optionally, the blocking oligonucleotides may comprise a blocking moiety at their 3′ end which prevents extension of said 3′ end by a polymerase. Any such blocking oligonucleotides may be present at a concentration of at least 1 nanomolar, at least 10 nanomolar, at least 100 nanomolar, or at least 1 micromolar.

One or more blocking oligonucleotides may be included together in the same solution as a chemical surfactant, and/or within the same solution as a molecular solvent, and/or within the same solution as a nucleic acid denaturant, and/or within the same solution as a library of multimeric barcoding reagents.

In the methods, after a step of annealing barcoded oligonucleotides to target nucleic acids, a blocking incubation may be performed to hybridise blocking oligonucleotides to complementary sequences within barcoded oligonucleotides. Optionally this blocking incubation may be performed at a temperature below the temperature at which barcoded oligonucleotides are annealed to nucleic acids from cells. Optionally this blocking incubation may be performed at a temperature below the temperature at which blocking oligonucleotides hybridise to complementary sequences within barcoded oligonucleotides.

In the methods, a nucleic acid size selection step may be performed after a step of annealing barcoded oligonucleotides to target nucleic acids. Optionally, this step may be performed by a gel-based size selection step. Optionally, this size selection step may be performed with a solid-phase reversible immobilisation process, such as a size selection step involving magnetic or superparamagnetic beads. Optionally, this size selection step may be performed with a column-based nucleic acid purification or size-selection step. Optionally, this size selection step may selectively or preferentially remove barcoded oligonucleotides that are not annealed or bound to nucleic acids from cells. Optionally, this size selection step may preferentially remove nucleic acid molecules less than 50 nucleotides in length, less than 100 nucleotides in length, less than 150 nucleotides in length, less than 200 nucleotides in length, less than 300 nucleotides in length, less than 400 nucleotides in length, less than 500 nucleotides in length, or less than 1000 nucleotides in length.

In the methods, after a step of annealing barcoded oligonucleotides to target nucleic acids, a primer extension step may be performed in the same high-viscosity solution in which the annealing was performed, wherein the barcoded oligonucleotides which are either attached to the solid support of the multimeric barcoding reagent or are in solution are extended using the target nucleic acid (e.g. messenger RNA) as a template. If the target nucleic acid is messenger RNA, this reaction may be a reverse transcription reaction carried out with a reverse transcriptase enzyme that is active in a high-viscosity solution.

In the methods, the multimeric barcoding reagents, barcoded oligonucleotides and/or multimeric barcode molecules may comprise one or more biotin moieties.

In the methods, following a step of annealing barcoded oligonucleotides to target nucleic acid from a cell sample, the barcoded oligonucleotides and/or barcoded target nucleic acid molecules and/or multimeric barcoding reagent(s) may be isolated by a process of: (a) contacting the resulting mixture with a solid support, optionally wherein the solid support comprises streptavidin moieties; and (b) capturing the barcoded oligonucleotides and/or barcoded target nucleic acid molecules and/or multimeric barcoding reagent(s) on the solid support, optionally through streptavidin-biotin interaction.

The solid support may be one or more magnetic beads, optionally wherein the one or more magnetic beads comprise streptavidin molecules on their surface. The solid support may comprise oligonucleotides capable of capturing the barcoded oligonucleotides or the target nucleic acid molecules. The barcoded oligonucleotides and/or barcoded target nucleic acid molecules and/or multimeric barcoding reagent may also be captured directly on the surface of the solid support, such as using solid phase reverse immobilization (SPRI) beads. The barcoded oligonucleotides and/or barcoded target nucleic acid molecules may also be re-captured on the multimeric barcoding reagent wherein the multimeric barcoding reagent itself comprises a solid support in the form of a magnetic bead. Optionally, the capture oligonucleotides may be complementary to one or more messenger RNA (mRNA) sequences. Optionally, the capture oligonucleotides may be complementary to the poly(A) tail sequence of messenger RNA (mRNA) sequences. Optionally, the capture oligonucleotides may comprise a poly(T) sequence of at least 2, at least 3, at least 5, at least 10, at least 20, at least 30, or at least 50 nucleotides that are complementary to the poly(A) tail sequence of messenger RNA (mRNA) sequences. Optionally, the capture oligonucleotides may be complementary to the barcoded oligonucleotides.

In the methods, the solution containing barcoded oligonucleotides and/or barcoded target nucleic acid molecules and/or multimeric barcoding reagent may be captured on a solid support, or the multimeric barcoding reagent may be first removed on a magnet and the supernatant containing barcoded oligonucleotides and/or barcoded target nucleic acid molecules may be captured on a solid support.

The magnetic bead(s) may be isolated from a reaction mixture with a magnet, or the magnetic beads carrying barcoded oligonucleotides and barcoded target nucleic acid molecules may be carried through into subsequent processing steps.

In subsequent processing steps (for example, after a step of isolating the annealed messenger RNA molecules and barcoded oligonucleotides), the messenger RNA may be reverse-transcribed with a reverse transcriptase, and then optionally amplified such as with a PCR process, prior to performing a sequencing reaction. The reverse transcription may include either and/or both first-strand reverse transcription (e.g. first-strand cDNA synthesis) and also second-strand synthesis, which may include random priming. Furthermore, any step of reverse transcription and/or cDNA synthesis may include any further standard step of cDNA processing, such as fragmentation (e.g. acoustic fragmentation such as Covaris sonication, or e.g. enzymatic fragmentation such as with a fragmentase enzyme, a restriction enzyme, and/or an in vitro transposase enzyme) and adapter (e.g. PCR adapter and/or sequencing adapter) ligation and/or adapter in vitro transposition at any stage(s) prior to and/or after reverse transcription and/or second strand synthesis and/or PCR. Any of these steps may be performed in solution, by eluting the original captured barcoded nucleic acids, or on-bead, with the barcoded nucleic acids still attached to the solid support used for capture in the previous step.

In the methods the reverse transcription reaction step includes an incubation step at which the reverse transcription uses the mRNA as a template for first strand synthesis. Optionally this can be performed at least 37° C., at least 42° C. or at least 55° C. Optionally, the duration of this incubation can be at least 15 minutes, or at least 30 minutes, or at least 45 minutes, or at least 1 hour. A portion of this reverse transcription reaction is use as the template for the second strand synthesis reaction. Optionally, this could be the whole reaction volume, or 1/10th of the reaction volume, or ⅕th of the reaction volume, or ¼th of the reaction volume, or ½ of the reaction volume, or ¾th of the reaction volume.

Second strand synthesis is then performed on the synthesised first strand, this may be done with the addition of an oligonucleotide containing an annealing site for the amplification PCR and a 7 random nucleotide sequence. Optionally, the length of random nucleotide sequence for this primer could be at least 5 nucleotides long, at least 6 nucleotides long, at least 8 nucleotides long, at least 9 nucleotides long or at least 10 nucleotides long. This primer is added along with the reverse transcription reaction to a master mix and heated in a stepwise fashion and cycled to allow annealing of the random primers and then extension of these with polymerase. Optionally the sequence of temperatures for annealing and extension could be 95° C. then 4° C. then 10° C. then 20° C. then 30° C. then 40° C. then 50° C. then 72° C., or it could be any of the previous temperatures in combination or separate to 98° C. then 5° C. then 15° C. then 25° C. then 35° C. then 45° C. then 55° C. then 68° C. The incubation times of each of these steps can be optionally initial incubation for 5 mins, then 3 cycles of 30 seconds, 3 minutes, 3 minutes, 3 minutes, 3 minutes, 3 minutes then 4 minutes. Or The incubation times of each of these steps can be optionally initial incubation for 5 mins, then 3 cycles of 30 seconds, 1 minute, 1 minute, 1 minute, 1 minute, 1 minute then 5 minutes. Or The incubation times of each of these steps can be optionally initial incubation for 5 mins, then 3 cycles of 30 seconds, 5 minutes, 5 minutes, 5 minutes, 5 minutes, 5 minutes then 4 minutes. Optionally the number of cycles of these temperature steps could be 1 cycle, 2 cycles, 3 cycles, 5 cycles or 10 cycles.

Upon completion and purification of the second strand synthesis a proportion of the second strand synthesis reaction is entered into a PCR reaction for amplification. Optionally, this could be the whole reaction volume, or 1/10th of the reaction volume, or ⅕th of the reaction volume, or ¼th of the reaction volume, or ½ of the reaction volume, or ¾th of the reaction volume. The PCR reaction will be run for a specific number of cycles before the final extension of PCR product. Optionally the number of cycles can include at least 20, or at least 25, or at least 30, or at least 35, or at least 40 cycles.

Upon completion and purification of the amplification PCR a proportion of the amplification reaction is entered into a sequencing adaptor attachment PCR. Optionally, this could be the whole amplification reaction volume, or ⅕th of the amplification reaction volume, or ¼th of the amplification reaction volume, or ½ of the amplification reaction volume, or ¾th of the amplification reaction volume. The sequencing adaptor attachment PCR will be run for a specific number of cycles before the final extension of PCR product. Optionally the number of cycles can include at least 4, or at least 6, or at least 8, or at least 10, or at least 15 cycles.

The sequencing adapters used in library preparation of the sample can be formatted to be compatible with next-generation DNA sequencing platforms, this is determined by the specific oligo sequences forming part of the primers used in the Sequencing adapter attachment PCR. Different next-generation DNA sequencing platforms include but are not limited to Illumina®, Pacific Biosciences™, Oxford Nanopore and BGI Genomics. This compatibility sequence may be from 5 bp to 100 bp long.

In the methods, the nucleic acid sample may comprise at least 2, at least 5, at least 10, at least 100, or at least 10³, at least 10⁴, at least 10⁵, at least 10⁶, at least 10⁷, at least 10⁸ or at least 10⁹ cells, wherein these cells are comprised within a single contiguous aqueous volume during any step of contacting the sample with a library of multimeric barcoding reagent (step (a)), and/or any step of lysing or permeabilising cells (step (b)), and/or any step of appending barcode sequences to target nucleic acids (steps (c), (d) and/or (e)). Preferably, in the methods, the nucleic acid sample comprises at least 10 cells, wherein these cells are comprised within a single contiguous aqueous volume during any step of contacting the sample with a library of multimeric barcoding reagent (step (a)), and any step of lysing or permeabilising cells (step (b)), and any step of appending barcode sequences to target nucleic acids (steps (c), (d) and/or (e))

Optionally, the nucleic acid sample may comprise at least 2, at least 5, at least 10, at least 100, or at least 10³, at least 10⁴, at least 10⁵, at least 10⁶, at least 10⁷, at least 10⁸ or at least 10⁹ cells, wherein these cells are partitioned within two or more contiguous aqueous volumes during any step of contacting the sample with a library of multimeric barcoding reagent (step (a)), and/or any step of lysing or permeabilising cells (step (b)), and/or any step of appending barcode sequences to target nucleic acids (steps (c), (d) and/or (e)).

In the methods, the nucleic acid sample may comprise at least 2, at least 5, at least 10, at least 100, or at least 10³, at least 10⁴, at least 10⁵, at least 10⁶, at least 10⁷, at least 10⁸ or at least 10⁹ cells, wherein these cells are not partitioned within two or more contiguous aqueous volumes during any step of contacting the sample with a library of multimeric barcoding reagent (step (a)), and/or any step of lysing or permeabilising cells (step (b)), and/or any step of appending barcode sequences to target nucleic acids (steps (c), (d) and/or (e)).

Optionally, barcoded target nucleic acid molecules are produced from target nucleic acids of at least 2, at least 5, at least 10, at least 100, or at least 10³, at least 10⁴, at least 10⁵, at least 10⁶, at least 10⁷, at least 10⁸ or at least 10⁹ cells.

Optionally the sequences of the barcoded target nucleic acid molecules produced for at least 10, at least 100, or at least 10³, at least 10⁴, at least 10⁵, at least 10⁶, at least 10⁷, at least 10⁸ or at least 10⁹ cells are determined.

In the methods, the library may comprise at least 100, or at least 10³, at least 10⁴, at least 10⁵, at least 10⁶, at least 10⁷, at least 10⁸ or at least 10⁹ multimeric barcoding reagents. In the methods, for each multimeric barcoding reagent, at least 2, at least 3, at least 5, at least 10, at least 25, at least 50, at least 100, at least 500, at least 1000, at least 5,000, at least 10,000, or at least 50,000 barcoded target nucleic acid molecules may be produced from the target nucleic acids of a single cell. Preferably, at least 2 barcoded target nucleic acid molecules may be produced from the target nucleic acids of a single cell for each multimeric barcoding reagent.

In the methods, each multimeric barcoding reagent may comprise at least 2, at least 3, at least 5, at least 10, at least 20, at least 50, at least 100, at least 200, at least 500, at least 1000, at least 5000, at least 10,000, at least 100,000, or at least 1,000,000 barcoded oligonucleotides. Optionally, different multimeric barcoding reagents within a library of multimeric barcoding reagents may comprise different numbers of barcoded oligonucleotides.

In the methods, on average, the barcoded oligonucleotides of a single multimeric barcoding reagent may anneal, cumulatively, to at least 1, at least 2, at least 3, at least 5, at least 10, at least 20, at least 50, at least 100, at least 200, at least 500, at least 1000, at least 5000, at least 10,000, or at least 100,000 target nucleic acids from cells.

In the methods, the group of target nucleic acid sequences complementary to the target regions of different barcoded oligonucleotides within a multimeric barcoding reagent or a library of multimeric barcoding reagents may comprise at least 2 different nucleic acid sequences, at least 3 different nucleic acid sequences, at least 4 different nucleic acid sequences, at least 5 different nucleic acid sequences, at least 10 different nucleic acid sequences, at least 20 different nucleic acid sequences, at least 50 different nucleic acid sequences, at least 100 different nucleic acid sequences, or at least 1000 different nucleic acid sequences.

In the methods, during any step(s), within a solution, volume, or reaction, cells may be present at particular concentrations within the solution volume, for example at concentrations of less than 10 picomolar, less than 1 picomolar, less than 100 femtomolar, less than 10 femtomolar, less than 1 femtomolar, less than 100 attomolar, less than 10 attomolar, or less than 1 attomolar.

In the methods, during any step(s), within a solution, volume, or reaction, multimeric barcoding reagents may be present at particular concentrations within the solution volume, for example at concentrations of at least 100 nanomolar, at least 10 nanomolar, at least 1 nanomolar, at least 100 picomolar, at least 10 picomolar, at least 1 picomolar, at least 100 femtomolar, at least 10 femtomolar, or at least 1 femtomolar.

In the methods, a sample comprising permeabilised, lysed, or intact cells, and/or comprising multimeric barcoding reagents, and/or comprising barcoded oligonucleotides, and/or comprising other oligonucleotide sequences, may be partitioned into two or more partition volumes. Optionally, said partition volumes may each comprise a different physical reaction vessel. Optionally, said partition volumes may each comprise a different droplet within an emulsion, such as different aqueous droplets within a water-in-oil emulsion. Such a partitioning event may take place before and/or during any one or more steps within any protocol. Following such a partitioning step, the reactions from two or more such partitions may be merged together to form a single reaction volume.

In the methods, the nucleic acid sample may comprise intact cells. The nucleic acid sample may comprise cells that have been partially degraded. The nucleic acid sample may comprise cells that have been partially permeabilised and/or fragmented. The nucleic acid sample may comprise cells that have been formalin crosslinked and paraffin embedded (ie, a FFPE sample). The nucleic acid sample may comprise cells that are contained within an intact tissue sample or section, or a partially intact tissue sample or section. The nucleic acid sample may comprise cells that have been processed through a tissue dissociation and/or tissue digestion process. Optionally, such a dissociation or digestion process may comprise digestion with a proteinase such as Proteinase K.

The nucleic acid sample may comprise cells that have been processed through a cell sorting process, such as a fluorescence activated cell sorting (FACS) process. The nucleic acid sample may comprise cells that are within a single cell suspension.

The nucleic acid sample may comprise lymphocytes, such as T cells, and/or B cells, and or a mixture of immune cells such as a sample of peripheral blood mononuclear cells (PBMCs). For example, a single multimeric barcoding reagent may be used to append barcode sequences to the sequences of a heavy chain immunoglobulin mRNA and a light chain immunoglobulin mRNA from the same single. Alternatively, a single multimeric barcoding reagent may be used to append barcode sequences to the sequences of an alpha chain mRNA and a beta chain mRNA of a T cell receptor.

Optionally, any single multimeric barcoding reagent (and/or any library of multimeric barcoding reagents) may be used to append barcode sequences to the sequences of two or more different target nucleic acids and/or two or more different types of target nucleic acids, for example, the poly(A) region of messenger RNA molecules and the constant region of an alpha chain mRNA and/or a beta chain mRNA of a T cell receptor, or the poly(A) region of messenger RNA molecules and genomic DNA sequences (such as repeat sequences in genomic DNA), or the poly(A) region of messenger RNA molecules and the constant region of an light chain mRNA and/or a heavy chain mRNA of a B cell receptor (and/or other immunoglobulin receptor), or the poly(A) region of messenger RNA molecules and oligonucleotide sequences within one or more barcoded affinity probes, or genomic DNA sequences (such as repeat sequences in genomic DNA) and oligonucleotide sequences within one or more barcoded affinity probes, or the poly(A) region of messenger RNA molecules and genomic DNA sequences (such as repeat sequences in genomic DNA) and oligonucleotide sequences within one or more barcoded affinity probes.

Optionally, two or more different target regions may be comprised within different barcoded oligonucleotides comprised within any single multimeric barcoding reagent (and/or comprised within any library of multimeric barcoding reagents), wherein said two or more different target regions are complementary to the sequences of two or more different target nucleic acids and/or two or more different types of target nucleic acids, for example, complementary to the poly(A) region of messenger RNA molecules and complementary to the constant region of an alpha chain mRNA and/or a beta chain mRNA of a T cell receptor, or complementary to the poly(A) region of messenger RNA molecules and complementary to genomic DNA sequences (such as complementary to repeat sequences in genomic DNA), or complementary to the poly(A) region of messenger RNA molecules and complementary to the constant region of an light chain mRNA and/or a heavy chain mRNA of a B cell receptor (and/or other immunoglobulin receptor), or complementary to the poly(A) region of messenger RNA molecules and complementary to oligonucleotide sequences within one or more barcoded affinity probes, or complementary to genomic DNA sequences (such as repeat sequences in genomic DNA) and complementary to oligonucleotide sequences within one or more barcoded affinity probes, or complementary to the poly(A) region of messenger RNA molecules and complementary to genomic DNA sequences (such as repeat sequences in genomic DNA) and complementary to oligonucleotide sequences within one or more barcoded affinity probes.

The nucleic acid sample may comprise tumour cells. Optionally, the sample may comprise tumour-infiltrating lymphocytes (TILs). Optionally, the sample may comprise tumour samples comprising both tumour cells and tumour-infiltrating lymphocytes. Optionally, the sample may comprise circulating tumour cells (CTCs). The nucleic acid sample may be a human sample.

The nucleic acid sample may comprise subcellular compartments of cells or products of cellular apoptosis or necrosis such as vesicles or other microparticles.

The target nucleic acid may be a (single) intact nucleic acid molecule of a cell, two or more fragments of a nucleic acid molecule of a cell (such fragments may be co-localised in the sample) or two or more nucleic acid molecules of a cell. Therefore, sub-sequences of a target nucleic acid of a cell may be sub-sequences of the same nucleic acid molecule, sub-sequences of different fragments of the same nucleic acid molecule, or sequences or sub-sequences of different nucleic acid molecules (for example, sequences of different messenger RNA molecules (or portions thereof) of a cell; e.g. first and second sub-sequences of a target nucleic acid of a cell may be first and second different messenger RNA molecules (or portions thereof) of a cell).

The target nucleic acid may comprise genomic DNA or mitochondrial DNA. The target nucleic acid may comprise RNA such as messenger RNA, or a non-coding RNA such as ribosomal RNA, transfer RNA, long non-coding RNA, microRNA, small interfering RNA, small nucleolar RNA, Piwi-interacting RNA or other small RNAs. The target nucleic acid may comprise an oligonucleotide sequence comprised within one or more barcoded affinity probe(s). Optionally, a target nucleic acid may comprise a combination of DNA and RNA, and/or of DNA and an oligonucleotide sequence comprised within one or more barcoded affinity probe(s), and/or of RNA and an oligonucleotide sequence comprised within one or more barcoded affinity probe(s). The target nucleic acid may comprise a combination of DNA, RNA, and an oligonucleotide sequence comprised within one or more barcoded affinity probe(s).

A sequence of a target nucleic acid and/or a sub-sequence of a target nucleic acid that is capable of annealing of ligating to a barcoded oligonucleotide may comprise a sequence complementary to one or more repeat sequences. Such repeat sequences may comprise microsatellite sequences and/or small tandem repeat sequences, such as dinucleotide repeats and/or trinucleotide repeats, and/or larger minisatellite regions such as those found around telomeres. Any repeat sequences may comprise interspersed DNA repeats such as retrotransposons, and/or long interspersed elements (LINES) and/or short interspersed elements (SINES) such as Alu repeats. Alternatively and/or additionally, any repeat sequences may be clustered regularly interspaced short palindromic repeats (CRISPR) and/or other palindromic repeats.

As used herein the term target nucleic acid refers to the nucleic acids present within cells and to copies or amplicons thereof. For example, where the target nucleic acid is genomic DNA, the term target nucleic acid means genomic DNA present in a cell and copies or amplicons thereof e.g. DNA molecules that may be prepared from the genomic DNA by a primer-extension reaction. As a further example, where the target nucleic acid is mRNA, the term target nucleic acid means mRNA present in the cell and copies or amplicons thereof e.g. cDNA synthesized from the mRNA by reverse transcription.

In any of the methods, the target nucleic acids may be DNA (e.g. genomic DNA) or RNA (e.g. mRNA). Such target nucleic acids may comprise DNA or RNA of any origin; for example they may comprise natural or unmodified genomic DNA or messenger RNA from an in vivo or in vitro sample of cells. Furthermore, they may comprise DNA or RNA of any sort of synthetic origin, such as DNA (and/or associated expressed RNA transcripts) from any sort of transfection or transduction method, such as linear or circular plasmids, viral transfection constructs, exogenously-administered DNA of any sort, exogenously-administered RNA of any sort (such as exogenously administered messenger RNA or short-interfering RNA or short-hairpin RNA), or CRISPR constructs and/or CRISPR expression constructs and/or derivatives thereof (e.g. a Cas9 nuclease and/or expressed version thereof, and/or a guide RNA and/or expressed version thereof). Furthermore, the target nucleic acids may comprise DNA and/or RNA sequences that comprise identifier or barcode sequences, wherein a sample of cells (e.g. an in vitro sample of cells or an in vivo population of cells) has been contacted and/or genetically modified with a pooled library of two or more different synthetic sequences, wherein each of said two or more synthetic sequences comprises an identifying sequence such as a barcode sequence (such as ‘Guide Barcode’ (GBC) sequences within expressed GBC transcripts within the Perturb-Seq protocol [Dixit et al., 2016, Cell 167, 1853-1866 and Adamson et al., 2016, Cell 167, 1867-1882], or identifying sequence barcodes from lentiviral expresion libraries [e.g. the murine DECIPHER lentiviral shRNA libraries, CELLECTA, Inc]). In such approaches, said identifying sequences, upon being barcoded and sequenced by any method described herein, may be used to determine which one or more (if any) synthetic sequences that a given cell within the sample or population of cells was contacted and/or genetically modified with.

In any of the methods, the target nucleic acids may comprise exogenously-administered nucleic acid sequences comprising barcode sequences within a barcoded affinity probe, wherein a barcoded affinity probe comprises at least one affinity moiety linked to at least one barcode sequence.

Optionally, any affinity moiety may comprise one or more of: an antibody, an antibody fragment, a light chain antibody fragment, a single-chain variable fragment (scFv), a peptide, a cell penetrating peptide, an aptamer, a DNA adptamer, and/or an RNA aptamer. Optionally, any one or more affinity moiety may comprise a moiety capable of binding to, and/or comprising high and/or specific affinity for, a specific protein, glycoprotein, post-translationally modified protein, and/or other chemical or molecular species. Optionally, any one or more such affinity moiety may comprise a moiety capable of binding to, and/or comprising high and/or specific affinity for, a specific protein, glycoprotein, post-translationally modified protein, and/or other chemical or molecular species comprised on the surface of a cell, and/or comprised within the cell membrane of a cell, and/or comprised within the cytoplasm of a cell, and/or comprised within the nucleus of a cell, and/or any combination thereof.

Any barcoded affinity probe may comprise a probe-barcode oligonucleotide, wherein said probe-barcode oligonucleotide comprises a barcode sequence associated with and/or identifying of the affinity moiety to which it is linked. Optionally, any such barcode sequence may comprise a sequence at least 1, at least 2, at least 3, at least 5, at least 10, at least 20, or at least 30 nucleotides in length. Optionally, all probe-barcode oligonucleotides linked with the same particular affinity moiety (e.g., the same particular antibody species specific for the same protein target) may comprise the same sequence (e.g. the same identifying barcode sequence). Optionally, probe-barcode oligonucleotides linked with the same particular affinity moiety (e.g., the same particular antibody species specific for the same protein target) may comprise two or more different sequences (e.g. two or more different identifying barcode sequences). Optionally, any probe-barcode oligonucleotide may comprise an adapter and/or coupling sequence, wherein said sequence is at least 1, at least 2, at least 3, at least 5, at least 10, at least 20, or at least 30 nucleotides in length. Optionally, any adapter and/or coupling sequence within a probe-barcode oligonucleotide may comprise a sequence complementary to a target region of a barcoded oligonucleotide comprised within any multimeric barcoding reagent and/or library thereof. Optionally, any adapter and/or coupling sequence within a probe-barcode oligonucleotide may comprise a poly(A) sequence 2 or more nucleotides in length. Optionally, any adapter and/or coupling sequence within a probe-barcode oligonucleotide may be comprised within the 3′ end, and/or within the 5′ end, of said probe-barcode oligonucleotide.

Any probe-barcode oligonucleotide and affinity moiety comprised within a barcoded affinity probe may be linked by any means. Optionally, a probe-barcode oligonucleotide and affinity moiety may be linked by a covalent bond (for example, such as LighteningLink antibody labelling kits from Innova Biosciences). Optionally, a probe-barcode oligonucleotide and affinity moiety may be linked by a non-covalent bond (using for example wherein an affinity moiety comprises a streptavidin domain, and wherein a probe-barcode oligonucleotide comprises a biotin moiety to generate a non-covalent biotin/streptavidin link).

Any one or more barcoded affinity probes may be contacted and/or incubated with a sample of cells wherein said barcoded affinity probes are at any concentration, for example at concentrations of at least 100 nanomolar, at least 10 nanomolar, at least 1 nanomolar, at least 100 picomolar, at least 10 picomolar, at least 1 picomolar, at least 100 femtomolar, at least 10 femtomolar, or at least 1 femtomolar. The concentrations may be 1 picomolar to 100 nanomolar, 10 picomolar to 10 nanomolar, or 100 picomolar to 1 nanomolar.

Optionally, a pool of two or more different barcoded affinity probes may be used in the methods. The pool (or library) may comprise: a first barcoded affinity probe comprising a first affinity moiety and a first probe-barcode oligonucleotide, wherein the first affinity moiety is capable of binding to, and/or comprising high and/or specific affinity for, a first target (e.g. a specific protein, a glycoprotein, a post-translationally modified protein, and/or other chemical or molecular species); and a second barcoded affinity probe comprising a second affinity moiety and a second probe-barcode oligonucleotide, wherein the second affinity moiety is capable of binding to, and/or comprising high and/or specific affinity for, a second target (e.g. a specific protein, a glycoprotein, a post-translationally modified protein, and/or other chemical or molecular species). The pool (or library) of barcoded affinity probes may be provided within a single solution. The pool (or library) of barcoded affinity probes may be contacted and/or incubated with cells. Optionally, the pool (or library) may comprise at least 3, at least 5, at least 10, at least 20, or at least 30 different barcoded affinity probes (e.g targeting at least 3, at least 5, at least 10, at least 20, or at least 30 different targets (e.g. specific proteins, glycoproteins, post-translationally modified proteins, and/or other chemical or molecular species)).

Optionally, the target nucleic acids may comprise probe-barcode oligonucleotide within barcoded affinity probes, wherein a sample of cells (e.g. an in vitro sample of cells or an in vivo population of cells) has been contacted and/or incubated with one or more such barcoded affinity probes. Optionally, a sample of cells may be chemically crosslinked (e.g. with formaldehyde) prior to any step of contacting and/or incubating cells with one or more barcoded affinity probes. Optionally, a sample of cells may be permeabilised (e.g. with a chemical surfactant) prior to any step of contacting and/or incubating cells with one or more barcoded affinity probes. Optionally, a sample of cells may be chemically crosslinked (e.g. with formaldehyde) and then permeabilised (e.g. with a chemical surfactant) prior to any step of contacting and/or incubating cells with one or more barcoded affinity probes.

Optionally, the target nucleic acids may comprise both nucleic acids comprised within a sample of cells and also probe-barcode oligonucleotide(s) within barcoded affinity probes, wherein the sample of cells (e.g. an in vitro sample of cells or an in vivo population of cells) has been contacted and/or incubated with one or more such barcoded affinity probes. Optionally, the target nucleic acids may comprise messenger RNA molecules comprised within a sample of cells and also probe-barcode oligonucleotide(s) within barcoded affinity probes, wherein the sample of cells (e.g. an in vitro sample of cells or an in vivo population of cells) has been contacted and/or incubated with one or more such barcoded affinity probes.

In the methods target nucleic acids from cells to which barcoded oligonucleotides anneal may comprise coupling sequences (e.g. synthetic nucleic acid sequences). Optionally, the target region of barcoded oligonucleotides within multimeric barcoding reagents may comprise sequences complementary to said coupling sequences to which they may anneal. Optionally, any said coupling sequences may comprise all or portions of synthetic oligonucleotides which have been transferred into cells within the nucleic acid sample. Optionally, such synthetic oligonucleotides may comprise a reagent-annealing region and a targeting region, wherein the reagent-annealing region is entirely or partially complementary to a target region within a barcoded oligonucleotide, and wherein the targeting region is entirely or partially complementary to a nucleic acid sequence found within the nucleic acid sample. Optionally, a targeting region may be entirely or partially complementary to a sequence within genomic DNA, or to a sequence within one or more messenger RNA (mRNA) molecules. Optionally, such synthetic oligonucleotides may comprise a linker region of at least 1 nucleotide between a reagent-annealing region and a targeting region. Optionally, the reagent-annealing region may be located within the 5′ end of a synthetic oligonucleotide and a targeting region may be located within the 3′ end of the synthetic oligonucleotide. Optionally, a solution of one or more synthetic oligonucleotides may be hybridised to one or more target nucleic acids within cells in a synthetic oligonucleotide annealing step. Optionally, such a synthetic oligonucleotide annealing step may be performed prior to contacting the sample of cells with a library of two or more multimeric barcoding reagents.

In the methods, the target nucleic acids from cells to which barcoded oligonucleotides anneal may be mRNA (messenger RNA) molecules. Optionally, the target region of barcoded oligonucleotides within multimeric barcoding reagents may comprise sequences complementary to sequences within one or more messenger RNA molecules to which they may anneal. Optionally, the target regions of barcoded oligonucleotides may be complementary to specific sequences within specific messenger RNA targets. Optionally, the target regions of barcoded oligonucleotides may be complementary to poly(A) tail regions of messenger RNA molecules; in this case the target regions of barcoded oligonucleotides may comprise a poly(T) region of two or more contiguous nucleotides

In the methods, each barcoded target nucleic acid molecule may be produced after isolation of the barcoded oligonucleotide annealed to a target mRNA molecule by extending the barcoded oligonucleotide using a reverse transcriptase and wherein the target mRNA molecule is employed as the template for a reverse transcription process by said reverse transcriptase.

In the methods, the mRNA molecules may be mRNA molecules corresponding to alpha and/or beta chains of a T-cell receptor sequence, optionally wherein the sequences of alpha and beta chains paired within an individual cell are determined.

In the methods, the mRNA molecules may be mRNA molecules corresponding to light and/or heavy chains of an immunoglobulin sequence, optionally wherein the sequences of light and heavy chains paired within an individual cell are determined.

In the methods, a blocking oligonucleotide may be used. A blocking oligonucleotide is capable of annealing to a specific DNA sequence in order to stop or ‘block’ an interaction between the target nucleic acid sequence and another oligonucleotide or complementary strand of DNA or RNA. These may be used to control the duration of a reaction by sequestering an active oligonucleotide in the form of a primer or barcoded oligonucleotide and can be controlled by physical addition or by temperature activation. These may be utilized in any one or more steps of the protocol for example indexing and or capture and or reverse transcription and or second strand synthesis and or PCR. An example of the use of a blocking oligonucleotide is in a lysis and indexing reaction step, an oligonucleotide with affinity to the barcoded oligonucleotides is used to block the barcoded oligonucleotides once the annealing step is completed, this stops the annealing reaction continuing after substantial diffusion of the barcoded oligonucleotides. The blocking oligo may be added at the point of lysis, but with the blocking mechanism triggered upon the lowering of the reaction temperature allowing it to be controlled. Another example is in a second strand synthesis reaction, where a blocking oligonucleotide may be designed to block a poly-T sequence produced in the second strand synthesis of indexed mRNA, and/or within any primer extension and/or PCR step and/or process.

Further details of the libraries of multimeric barcoding reagents and methods of the invention are provided below.

### 1. GENERAL PROPERTIES OF MULTIMERIC BARCODING REAGENTS

The invention provides multimeric barcoding reagents for labelling one or more target nucleic acids. A multimeric barcoding reagent comprises two or more barcode regions are linked together (directly or indirectly).

Each barcode region comprises a nucleic acid sequence. The nucleic acid sequence may be single-stranded DNA, double-stranded DNA, or single stranded DNA with one or more double-stranded regions.

Each barcode region may comprise a sequence that identifies the multimeric barcoding reagent. For example, this sequence may be a constant region shared by all barcode regions of a single multimeric barcoding reagent. Each barcode region may contain a unique sequence which is not present in other regions, and may thus serve to uniquely identify each barcode region. Each barcode region may comprise at least 5, at least 10, at least 15, at least 20, at least 25, at least 50 or at least 100 nucleotides. Preferably, each barcode region comprises at least 5 nucleotides. Preferably each barcode region comprises deoxyribonucleotides, optionally all of the nucleotides in a barcode region are deoxyribonucleotides. One or more of the deoxyribonucleotides may be a modified deoxyribonucleotide (e.g. a deoxyribonucleotide modified with a biotin moiety or a deoxyuracil nucleotide). The barcode regions may comprise one or more degenerate nucleotides or sequences. The barcode regions may not comprise any degenerate nucleotides or sequences.

The multimeric barcoding reagent may comprise at least 5, at least 10, at least 20, at least 25, at least 50, at least 75, at least 100, at least 200, at least 500, at least 1000, at least 5000, or at least 10,000 barcode regions. Preferably, the multimeric barcoding reagent comprises at least 5 barcode regions.

The multimeric barcoding reagent may comprise at least 2, at least 3, at least 4, at least 5, at least 10, at least 20, at least 25, at least 50, at least 75, at least 100, at least 200, at least 500, at least 1000, at least 5000, at least 10⁴, at least 10⁵, or at least 10⁶ unique or different barcode regions. Preferably, the multimeric barcoding reagent comprises at least 5 unique or different barcode regions.

A multimeric barcoding reagent may comprise: first and second barcode molecules linked together (i.e. a multimeric barcode molecule), wherein each of the barcode molecules comprises a nucleic acid sequence comprising a barcode region.

The barcode molecules of a multimeric barcode molecule may be linked on a nucleic acid molecule (e.g. a single-stranded oligonucleotide). The barcode molecules of a multimeric barcode molecule may be comprised within a (single) nucleic acid molecule. A multimeric barcode molecule may comprise a single, contiguous nucleic acid sequence comprising two or more barcode molecules. A multimeric barcode molecule may be a single-stranded nucleic acid molecule (e.g. single-stranded DNA), a double-stranded-stranded nucleic acid molecule or a single stranded molecule comprising one or more double-stranded regions. A multimeric barcode molecule may comprise one or more phosphorylated 5′ ends capable of ligating to 3′ ends of other nucleic acid molecules. Optionally, in a double-stranded region or between two different double-stranded regions, a multimeric barcode molecule may comprise one or more nicks, or one or more gaps, where the multimeric barcode molecule itself has been divided or separated. Any said gap may be at least one, at least 2, at least 5, at least 10, at least 20, at least 50, or at least 100 nucleotides in length. Said nicks and/or gaps may serve the purpose of increasing the molecular flexibility of the multimeric barcode molecule and/or multimeric barcoding reagent, for example to increase the accessibility of the molecule or reagent to interact with target nucleic acid molecules. Said nicks and/or gaps may also enable more efficient purification or removal of said molecules or reagents. A molecule and/or reagent comprising said nick(s) and/or gap(s) may retain links between different barcode molecules by having a complementary DNA strand which is jointly hybridised to regions of two or more divided parts of a multimeric barcode molecule.

A multimeric barcode molecule may comprise natural nucleotides and/or it could also contain chemical modifications like linkers and chemical attachment sites. The multimeric barcode molecule may be produced by phosphoramidite-based oligonucleotide synthesis. This method may allow the introduction of chemical modifications. The length of oligonucleotide produced by this method may be increased by using extendible oligonucleotides, wherein ligation chemistries can be used to elongate the short synthetic oligonucleotides. The ligation process may be performed in solution or on a surface. The process may be an enzymatic or chemical process. If a chemical process is used, cyclically alternating orthogonal ligation chemistries may be used (e.g. to avoid intra-strand ligation). The multimeric barcode molecule may be produced by rolling circle amplification (RCA). This may involve using a short cyclical template containing the desired sequence to generate a long oligonucleotide containing repeat sequences of the desired sequence. For example, each of these repeat sequences may comprise a barcode molecule. A specific sequence may be included at the end of a multimeric hybridization molecule by using a terminal transferase or by using a non-templated ligase.

The barcode molecules may be linked by a support e.g. a macromolecule, solid support or semi-solid support. The sequences of the barcode molecules linked to each support may be known. The barcode molecules may be linked to the support directly or indirectly (e.g. via a linker molecule). The barcode molecules may be linked by being bound to the support and/or by being bound or annealed to linker molecules that are bound to the support. The barcode molecules may be bound to the support (or to the linker molecules) by covalent linkage, non-covalent linkage (e.g. a protein-protein interaction or a streptavidin-biotin bond) or nucleic acid hybridization. The linker molecule may be a biopolymer (e.g. a nucleic acid molecule) or a synthetic polymer. The linker molecule may comprise one or more units of ethylene glycol and/or poly(ethylene) glycol (e.g. hexa-ethylene glycol or penta-ethylene glycol). The linker molecule may comprise one or more ethyl groups, such as a C3 (three-carbon) spacer, C6 spacer, C12 spacer, or C18 spacer. The linker molecule may comprise at least 2, at least 3, at least 4, at least 5, at least 10, or at least 20 sequential repeating units of any individual linker (such as a sequential linear series of at least 2, at least 5, or at least 10 C12 spacers or C18 spacers). The linker molecule may comprise a branched linker molecule, wherein 2 or more barcode molecules are linked to a support by a single linker molecule.

The barcode molecules may be linked by a macromolecule by being bound to the macromolecule and/or by being annealed to the macromolecule.

The barcode molecules may be linked to the macromolecule directly or indirectly (e.g. via a linker molecule). The barcode molecules may be linked by being bound to the macromolecule and/or by being bound or annealed to linker molecules that are bound to the macromolecule. The barcode molecules may be bound to the macromolecule (or to the linker molecules) by covalent linkage, non-covalent linkage (e.g. a protein-protein interaction or a streptavidin-biotin bond) or nucleic acid hybridization. The linker molecule may be a biopolymer (e.g. a nucleic acid molecule) or a synthetic polymer. The linker molecule may comprise one or more units of ethylene glycol and/or poly(ethylene) glycol (e.g. hexa-ethylene glycol or penta-ethylene glycol). The linker molecule may comprise one or more ethyl groups, such as a C3 (three-carbon) spacer, C6 spacer, C12 spacer, or C18 spacer.

The macromolecule may be a synthetic polymer (e.g. a dendrimer) or a biopolymer such as a nucleic acid (e.g. a single-stranded nucleic acid such as single-stranded DNA), a peptide, a polypeptide or a protein (e.g. a multimeric protein).

The dendrimer may comprise at least 2, at least 3, at least 5, or at least 10 generations.

The macromolecule may be a nucleic acid comprising two or more nucleotides each capable of binding to a barcode molecule. Additionally or alternatively, the nucleic acid may comprise two or more regions each capable of hybridizing to a barcode molecule.

The nucleic acid may comprise a first modified nucleotide and a second modified nucleotide, wherein each modified nucleotide comprises a binding moiety (e.g. a biotin moiety, or an alkyne moiety which may be used for a click-chemical reaction) capable of binding to a barcode molecule. Optionally, the first and second modified nucleotides may be separated by an intervening nucleic acid sequence of at least one, at least two, at least 5 or at least 10 nucleotides.

The nucleic acid may comprise a first hybridization region and a second hybridization region, wherein each hybridization region comprises a sequence complementary to and capable of hybridizing to a sequence of at least one nucleotide within a barcode molecule. The complementary sequence may be at least 5, at least 10, at least 15, at least 20, at least 25 or at least 50 contiguous nucleotides. Preferably, the complementary sequence is at least 10 contiguous nucleotides. Optionally, the first and second hybridization regions may be separated by an intervening nucleic acid sequence of at least one, at least two, at least 5 or at least 10 nucleotides.

The macromolecule may be a protein such as a multimeric protein e.g. a homomeric protein or a heteromeric protein. For example, the protein may comprise streptavidin e.g. tetrameric streptavidin.

The support may be a solid support or a semi-solid support. The support may comprise a planar surface. The support may be a slide e.g. a glass slide. The slide may be a flow cell for sequencing. If the support is a slide, the first and second barcode molecules may be immobilized in a discrete region on the slide. Optionally, the barcode molecules of each multimeric barcoding reagent in a library are immobilized in a different discrete region on the slide to the barcode molecules of the other multimeric barcoding reagents in the library. The support may be a plate comprising wells, optionally wherein the first and second barcode molecules are immobilized in the same well. Optionally, the barcode molecules of each multimeric barcoding reagent in library are immobilized in a different well of the plate to the barcode molecules of the other multimeric barcoding reagents in the library.

Preferably, the support is a bead (e.g. a gel bead). The bead may be a polymer-based bead, an agarose bead, a silica bead, a styrofoam/polystyrene bead, a dextran bead, a polylactic acid bead, a polyvinyl alcohol bead, a gel bead (such as those available from 10× Genomics®), an antibody conjugated bead, an oligo-dT conjugated bead, a streptavidin bead or a magnetic bead (e.g. a superparamagnetic bead). The bead may be a microbead (e.g. a magnetic microbead). The bead may be of any size and/or molecular structure. For example, the bead may be 10 nanometres to 200 microns in diameter, 10 nanometres to 100 microns in diameter, 100 nanometres to 10 microns in diameter, 1 micron to 5 microns in diameter or 10 microns to 50 microns in diameter. Optionally, the bead is approximately 10 nanometres in diameter, approximately 100 nanometres in diameter, approximately 1 micron in diameter, approximately 10 microns in diameter or approximately 100 microns in diameter. The bead may be solid, or alternatively the bead may be hollow or partially hollow or porous. Beads of certain sizes may be most preferable for certain barcoding methods. For example, beads less than 35.0 microns, less than 5.0 microns, or less than 1.0 micron, may be most useful for barcoding nucleic acid targets within individual cells. Preferably, the barcode molecules of each multimeric barcoding reagent in a library are linked together on a different bead to the barcode molecules of the other multimeric barcoding reagents in the library.

The support may be functionalised to enable attachment of two or more barcode molecules. This functionalisation may be enabled through the addition of chemical moieties (e.g. carboxylated groups, alkynes, azides, acrylate groups, amino groups, sulphate groups, tosyl groups, epoxy groups, thiols, maleimides, iodoacetyl groups, Au for thiol-Au based approaches or succinimide/NHS ester groups), and/or protein-based moieties (e.g. streptavidin, avidin, or protein G) to the support. The barcode molecules may be attached to the moieties directly or indirectly (e.g. via a linker molecule).

Barcoded oligonucleotides and/or multimeric barcode molecules may be linked to a support by amine-carboxylic acid/NHS-ester peptide coupling, azide-alkyne click chemistry (e.g. CuAAC or SPAAC), non-covalent interaction (e.g. streptavidin-biotin or thiol-based approaches such as thiol-maleimide), disulfide and thiol-Au interactions.

Functionalised supports (e.g. beads) may be brought into contact with a solution of barcode molecules under conditions which promote the attachment of two or more barcode molecules to each bead in the solution (generating multimeric barcoding reagents).

In a library of multimeric barcoding reagents, the barcode molecules of each multimeric barcoding reagent in a library may be linked together on a different support to the barcode molecules of the other multimeric barcoding reagents in the library.

The multimeric barcoding reagent may comprise: at least 2, at least 3, at least 4, at least 5, at least 10, at least 20, at least 25, at least 50, at least 75, at least 100, at least 200, at least 500, at least 1000, at least 5000, at least 10⁴, at least 10⁵, at least 10⁶, at least 10⁷, at least 10⁸, at least 10⁹ or at least 10¹⁰ barcode molecules linked together, wherein each barcode molecule is as defined herein; and a barcoded oligonucleotide annealed to each barcode molecule, wherein each barcoded oligonucleotide is as defined herein. Preferably, the multimeric barcoding reagent comprises at least 5 barcode molecules linked together, wherein each barcode molecule is as defined herein; and a barcoded oligonucleotide annealed to each barcode molecule, wherein each barcoded oligonucleotide is as defined herein.

The multimeric barcoding reagent may comprise: at least 2, at least 3, at least 4, at least 5, at least 10, at least 20, at least 25, at least 50, at least 75, at least 100, at least 200, at least 500, at least 1000, at least 5000, at least 10⁴, at least 10⁵, at least 10⁶, at least 10⁷, at least 10⁸, at least 10⁹ or at least 10¹⁰ unique or different barcode molecules linked together, wherein each barcode molecule is as defined herein; and a barcoded oligonucleotide annealed to each barcode molecule, wherein each barcoded oligonucleotide is as defined herein. Preferably, the multimeric barcoding reagent comprises at least 5 unique or different barcode molecules linked together, wherein each barcode molecule is as defined herein; and a barcoded oligonucleotide annealed to each barcode molecule, wherein each barcoded oligonucleotide is as defined herein.

A multimeric barcoding reagent may comprise two or more barcoded oligonucleotides as defined herein, wherein the barcoded oligonucleotides each comprise a barcode region. A multimeric barcoding reagent may comprise: at least 2, at least 3, at least 4, at least 5, at least 10, at least 20, at least 25, at least 50, at least 75, at least 100, at least 200, at least 500, at least 1000, at least 5000, at least 10⁴, at least 10⁵, at least 10⁶, at least 10⁷, at least 10⁸, at least 10⁹ or at least 10¹⁰ unique or different barcoded oligonucleotides. Preferably, the multimeric barcoding reagent comprises at least 5 unique or different barcoded oligonucleotides.

The barcoded oligonucleotides of a multimeric barcoding reagent are linked together (directly or indirectly). The barcoded oligonucleotides of a multimeric barcoding reagent are linked together by a support e.g. a macromolecule, solid support or semi-solid support, as described herein. The barcoded oligonucleotides of a multimeric barcoding reagent may be linked to the support covalently, non-covalently, electrostatically, via Van der Waals forces and/or hydrophobic interactions, via physisorption and/or chemisorption. For example, barcoded Oligonucleotides may be linked to the support via a cleavable linker or through hybridization to a second oligonucleotide (e.g. a multimeric hybridization molecule or multimeric barcode molecule). The second oligonucleotide may in turn be linked to the support covalently, non-covalently, electrostatically, via Van der Waals forces and/or hydrophobic interactions, via physisorption and/or chemisorption.

The multimeric barcoding reagent may comprise one or more polymers to which the barcoded oligonucleotides are annealed or attached. For example, the barcoded oligonucleotides of a multimeric barcoding reagent may be annealed to a multimeric hybridization molecule e.g. a multimeric barcode molecule. Alternatively, the barcoded oligonucleotides of a multimeric barcoding reagent may be linked together by a macromolecule (such as a synthetic polymer e.g. a dendrimer, or a biopolymer e.g. a protein) or a support (such as a solid support or a semi-solid support e.g. a gel bead). Additionally or alternatively, the barcoded oligonucleotides of a (single) multimeric barcoding reagent may linked together by being comprised within a (single) lipid carrier (e.g. a liposome or a micelle).

A multimeric barcoding reagent may comprise: first and second hybridization molecules linked together (i.e. a multimeric hybridization molecule), wherein each of the hybridization molecules comprises a nucleic acid sequence comprising a hybridization region; and first and second barcoded oligonucleotides, wherein the first barcoded oligonucleotide is annealed to the hybridization region of the first hybridization molecule and wherein the second barcoded oligonucleotide is annealed to the hybridization region of the second hybridization molecule.

The barcoded oligonucleotides annealed to a multimeric hybridization molecule may be the same or different. If they are different, each multimeric barcoding reagent may comprise only a single copy of each different barcoded oligonucleotide or multiple copies of each different barcoded oligonucleotide.

Each multimeric barcoding reagent may comprise at least 5, at least 10, at least 20, at least 50, at least 100, at least 10³, at least 10⁴, at least 10⁵, at least 10⁶, at least 10⁷, at least 10⁸, at least 10⁹ or at least 10¹⁰ multimeric hybridization molecules, wherein each multimeric hybridization molecule is independently linked to the same single support. Preferably, each multimeric barcoding reagent comprises at least 10⁴ multimeric hybridization molecules, wherein each multimeric hybridization molecule is independently linked to the same single support.

Each multimeric barcoding reagent may comprise at least 5, at least 10, at least 20, at least 50, at least 100, at least 10³, at least 10⁴, at least 10⁵, at least 10⁶, at least 10⁷, at least 10⁸, at least 10⁹ or at least 10¹⁰ barcoded oligonucleotides annealed to each of the multimeric hybridization molecules. Preferably, each multimeric barcoding reagent comprises at least 5 barcoded oligonucleotides annealed to each of the multimeric hybridization molecules.

Each multimeric barcoding reagent may comprise at least 5, at least 10, at least 20, at least 50, at least 100, at least 10³, at least 10⁴, at least 10⁵, at least 10⁶, at least 10⁷ or at least 10⁸, or at least 10⁹ barcoded oligonucleotides annealed to the multimeric hybridization molecules that are independently linked to the same single support. Preferably, each multimeric barcoding reagent comprises at least 10⁴ barcoded oligonucleotides annealed to the multimeric hybridization molecules that are independently linked to the same single support.

Each multimeric barcoding reagent may comprise at least 2, at least 5, at least 10, at least 20, at least 50, at least 100, at least 10³, at least 10⁴, at least 10⁵, at least 10⁶, at least 10⁷ or at least 10⁸ unique barcoded oligonucleotides annealed to each of the multimeric hybridization molecules. Preferably, each multimeric barcoding reagent comprises at least 2 unique barcoded oligonucleotides annealed to each of the multimeric hybridization molecules. Each multimeric barcoding reagent may comprise at least 2, at least 5, at least 10, at least 20, at least 50, at least 100, at least 10³, at least 10⁴, at least 10⁵, at least 10⁶, at least 10⁷ or at least 10⁸ copies of each unique barcoded oligonucleotide annealed to each of the multimeric hybridization molecules. Preferably, each multimeric barcoding reagent comprises at least 10 copies of each unique barcoded oligonucleotide annealed to each of the multimeric hybridization molecules.

Each multimeric barcoding reagent may comprise at least 5, at least 10, at least 20, at least 50, at least 100, at least 1000, at least 5000, at least 10⁴, at least 10⁵, at least 10⁶, at least 10⁷ or at least 10⁸ unique barcoded oligonucleotides annealed to the multimeric hybridization molecules that are independently linked to the same single support. Preferably, each multimeric barcoding reagent comprises at least 10 unique barcoded oligonucleotides annealed to the multimeric hybridization molecules that are independently linked to the same single support. Each multimeric barcoding reagent may comprise at least 2, at least 5, at least 10, at least 20, at least 50, at least 100, at least 10³, at least 10⁴, at least 10⁵, at least 10⁶, at least 10⁷ or at least 10⁸ copies of each unique barcoded oligonucleotide annealed to the multimeric hybridization molecules that are independently linked to the same single support. Preferably, each multimeric barcoding reagent comprises at least 10⁴ copies of each unique barcoded oligonucleotide annealed to the multimeric hybridization molecules that are independently linked to the same single support.

The hybridization regions of a multimeric hybridization molecule may be contiguous (i.e. repeated immediately after each other) or they may be separated by a linker. The hybridization regions may be identical or they may be different to each other (either uniquely or in repeated groups), to allow selective annealing of different barcoded oligonucleotides.

A multimeric hybridization molecule may further comprise a cell-binding moiety or may be linked to a cell-binding moiety. A multimeric hybridization molecule may further comprise one or more regions that anneal to a cell-binding oligonucleotide. Each cell-binding oligonucleotide may be linked to a cell-binding moiety.

The hybridization molecules comprise or consist of deoxyribonucleotides. One or more of the deoxyribonucleotides may be a modified deoxyribonucleotide (e.g. a deoxyribonucleotide modified with a biotin moiety or a deoxyuracil nucleotide). The hybridization molecules may comprise one or more degenerate nucleotides or sequences. The hybridization molecules may not comprise any degenerate nucleotides or sequences.

A multimeric hybridization molecule may comprise natural nucleotides and/or it could also contain chemical modifications like linkers and chemical attachment sites. The multimeric hybridization molecule may be produced by phosphoramidite-based oligonucleotide synthesis. This method may allow the introduction of chemical modifications. The length of oligonucleotide produced by this method may be increased by using extendible oligonucleotides, wherein ligation chemistries can be used to elongate the short synthetic oligonucleotides. The ligation process may be performed in solution or on a surface. The process may be an enzymatic or chemical process. If a chemical process is used, cyclically alternating orthogonal ligation chemistries may be used (e.g. to avoid intra-strand ligation). The multimeric hybridization molecule may be produced by rolling circle amplification (RCA). This may involve using a short cyclical template containing the desired sequence to generate a long oligonucleotide containing repeat sequences of the desired sequence. For example, each of these repeat sequences may comprise a hybridization molecule. A specific sequence may be included at the end of a multimeric hybridization molecule by using a terminal transferase or by using a non-templated ligase.

The hybridization molecules of a multimeric hybridization molecule may be linked on a nucleic acid molecule (e.g. a single-stranded oligonucleotide). Such a nucleic acid molecule may provide the backbone to which single-stranded barcoded oligonucleotides may be annealed. The hybridization molecules of a multimeric hybridization molecule may be comprised within a (single) nucleic acid molecule. A multimeric hyrbidization molecule may comprise a single, contiguous nucleic acid sequence comprising two or more hybridization molecules. A multimeric hybridization molecule may be a single-stranded nucleic acid molecule (e.g. single-stranded DNA) comprising two or more hybridization molecules. A multimeric hybridization molecule may comprise one or more double-stranded regions. Optionally, in a double-stranded region or between two different double-stranded regions, a multimeric hybridization molecule may comprise one or more nicks, or one or more gaps, where the multimeric hybridization molecule itself has been divided or separated. Any said gap may be at least one, at least 2, at least 5, at least 10, at least 20, at least 50, or at least 100 nucleotides in length. Said nicks and/or gaps may serve the purpose of increasing the molecular flexibility of the multimeric hybridization molecule and/or multimeric barcoding reagent, for example to increase the accessibility of the molecule or reagent to interact with target nucleic acid molecules. Said nicks and/or gaps may also enable more efficient purification or removal of said molecules or reagents. A molecule and/or reagent comprising said nick(s) and/or gap(s) may retain links between different hybridization molecules by having a complementary DNA strand which is jointly hybridised to regions of two or more divided parts of a multimeric hybridization molecule.

The hybridization molecules may be linked by a macromolecule by being bound to the macromolecule and/or by being annealed to the macromolecule.

The hybridization molecules may be linked to the macromolecule directly or indirectly (e.g. via a linker molecule). The hybridization molecules may be linked by being bound to the macromolecule and/or by being bound or annealed to linker molecules that are bound to the macromolecule. The hybridization molecules may be bound to the macromolecule (or to the linker molecules) by covalent linkage, non-covalent linkage (e.g. a protein-protein interaction or a streptavidin-biotin bond) or nucleic acid hybridization. The linker molecule may be a biopolymer (e.g. a nucleic acid molecule) or a synthetic polymer. The linker molecule may comprise one or more units of ethylene glycol and/or poly(ethylene) glycol (e.g. hexa-ethylene glycol or penta-ethylene glycol). The linker molecule may comprise one or more ethyl groups, such as a C3 (three-carbon) spacer, C6 spacer, C12 spacer, or C18 spacer.

The macromolecule may be a synthetic polymer (e.g. a dendrimer) or a biopolymer such as a nucleic acid (e.g. a single-stranded nucleic acid such as single-stranded DNA), a peptide, a polypeptide or a protein (e.g. a multimeric protein).

The dendrimer may comprise at least 2, at least 3, at least 5, or at least 10 generations.

The macromolecule may be a nucleic acid comprising two or more nucleotides each capable of binding to a hybridization molecule. Additionally or alternatively, the nucleic acid may comprise two or more regions each capable of hybridizing to a hybridization molecule.

The nucleic acid may comprise a first modified nucleotide and a second modified nucleotide, wherein each modified nucleotide comprises a binding moiety (e.g. a biotin moiety, or an alkyne moiety which may be used for a click-chemical reaction) capable of binding to a hybridization molecule. Optionally, the first and second modified nucleotides may be separated by an intervening nucleic acid sequence of at least one, at least two, at least 5 or at least 10 nucleotides.

The nucleic acid may comprise a first hybridization region and a second hybridization region, wherein each hybridization region comprises a sequence complementary to and capable of hybridizing to a sequence of at least one nucleotide within a hybridization molecule. The complementary sequence may be at least 5, at least 10, at least 15, at least 20, at least 25 or at least 50 contiguous nucleotides. Optionally, the first and second hybridization regions may be separated by an intervening nucleic acid sequence of at least one, at least two, at least 5 or at least 10 nucleotides.

The macromolecule may be a protein such as a multimeric protein e.g. a homomeric protein or a heteromeric protein. For example, the protein may comprise streptavidin e.g. tetrameric streptavidin.

The hybridization molecules may be linked by a support. The hybridization molecules may be linked to the support directly or indirectly (e.g. via a linker molecule). The hybridization molecules may be linked by being bound to the support and/or by being bound or annealed to linker molecules that are bound to the support. The hybridization molecules may be bound to the support (or to the linker molecules) by covalent linkage, non-covalent linkage (e.g. a protein-protein interaction or a streptavidin-biotin bond) or nucleic acid hybridization. The linker molecule may be a biopolymer (e.g. a nucleic acid molecule) or a synthetic polymer. The linker molecule may comprise one or more units of ethylene glycol and/or poly(ethylene) glycol (e.g. hexa-ethylene glycol or penta-ethylene glycol). The linker molecule may comprise one or more ethyl groups, such as a C3 (three-carbon) spacer, C6 spacer, C12 spacer, or C18 spacer.

A multimeric barcoding reagent may comprise at least two multimeric hybridization molecules (e.g. at least two multimeric barcode molecules) linked to a support.

A multimeric hybridization molecule (e.g. a multimeric barcode molecule) may be linked to a support either directly or indirectly (e.g. via one or more linker molecules). A multimeric hybridization molecule (e.g. a multimeric barcode molecule) may be linked to a support via a linker molecule, wherein said linker molecule is appended to and/or linked to and/or bound to (covalently or non-covalently) both at least one support, and at least one multimeric hybridization molecule. A multimeric hybridization molecule (e.g. a multimeric barcode molecule) may be linked to any support by one or more covalent linkage(s) (or bond(s)) (e.g. by a covalent bond such as a bond generated by any amino-modification attachment chemistry, and/or any carboxy-modification attachment chemistry, and/or any thiol-modification attachment chemistry, and/or any NHS-ester attachment chemistry, and/or any click-chemistry-related method, such as any copper (I)-catalysed azide-alkyne cycloaddition (CuAAC) reaction, a strain-promoted azide-alkyne cycloaddition (SPAAC) reaction, a strain-promoted alkyne-nitrone cycloaddition (SPANC) reaction, or an alkene and tetrazole photoclick reaction), one or more non-covalent linkages (or bond(s)) (e.g. a protein-protein interaction or a streptavidin-biotin linkage e.g. a support may comprise a streptavidin domain and a multimeric hybridization molecule (e.g. a multimeric barcode molecule) may comprise a biotin moiety) or a nucleic acid hybridization linkage. Any one or more linker molecule may be a biopolymer (e.g. a nucleic acid molecule, peptide, etc.) or a synthetic polymer. Any one or more linker molecule may comprise one or more units of ethylene glycol and/or poly(ethylene) glycol (e.g. hexa-ethylene glycol or penta-ethylene glycol). Any one or more linker molecule may comprise one or more ethyl groups, such as one or more C3 (three-carbon) spacers, C6 spacers, C12 spacers, or C18 spacers. Any one or more linker molecule may be made of any other chemistry or polymer (e.g. peptide based polymers, nucleic acid based polymers like for example PNA, any synthetic polymer, etc.).

Optionally, any one or more multimeric hybridization molecule (e.g. a multimeric barcode molecule), hybridization molecule, barcode molecule, barcoded oligonucleotide and/or adapter oligonucleotide may be (directly or indirectly) linked to and/or comprise one or more structural modifications, such as one or more natural or unnatural nucleotide and DNA modification (e.g. LNA, amino LNA, PNA, triazole backbone, amino backbone, 2′-O-methyl and/or 2′-O-methoxy-ethyl nucleosides, 2′-F and/or 2′-F-arabino nucleosides, phosphorothioates, modified bases (such as 2-aminopurine, tricyclic cytosines, 5-bromo dU, 8-oxoguanine, 5-methylcytosine etc.), fluorophores, intercalators, groove binders, etc.), such as one or more attachment modification (e.g. amino groups, carboxy groups, NHS esters, azido groups, alkyne groups, thiol groups, biotin/desthiobiotin, etc.), such as one or more brancher modification and/or linker molecules and/or linker moieties, such as one or more units of ethylene glycol and/or poly(ethylene) glycol (e.g. hexa-ethylene glycol and/or penta-ethylene glycol and/or PEG 500, PEG 1000, PEG 2000, PEG 4000, PEG 5000, PEG 10,000, and/or PEG 20,000; optionally, any such PEG moiety may have any degree of dispersity in size/molecular mass, i.e. any degree of monodispersity or polydispersity), and/or any one or more C3 (three-carbon) spacers, C6 spacers, C12 spacers, or C18 spacers. Optionally, any number of one or more such structural modifications (e.g. linker molecules and/or linker moieties) may be added to any individual multimeric hybridization molecule (e.g. a multimeric barcode molecule), hybridization molecule, barcode molecule, barcoded oligonucleotide and/or adapter oligonucleotide, such as at least 2 linker molecules and/or linker moieties, at least 3 linker molecules and/or linker moieties, at least 4 linker molecules and/or linker moieties, at least 5 linker molecules and/or linker moieties, at least 6 linker molecules and/or linker moieties, at least 8 linker molecules and/or linker moieties, at least 10 linker molecules and/or linker moieties, at least 15 linker molecules and/or linker moieties, at least 20 linker molecules and/or linker moieties, at least 30 linker molecules and/or linker moieties, at least 40 linker molecules and/or linker moieties, at least 50 linker molecules and/or linker moieties, or at least 100 or more linker molecules and/or linker moieties. Optionally, any such linker molecules and/or linker moieties may comprise branched linker molecules or linker moieties (such as a branched linker molecule comprising two or more ethyl groups, such as two or more spacer moieties, such as two or more C3 (three-carbon) spacers, and/or C6 spacers, and/or C12 spacers, and/or C18 spacers. Optionally, any such linker molecules and/or linker moieties may comprise sequentially-connected (i.e. linear) linker molecules or linker moieties (such as a sequential repeating units of a linker molecule comprising two or more ethyl groups in linear series, such as two or more spacer moieties, such as two or more C3 (three-carbon) spacers, and/or C6 spacers, and/or C12 spacers, and/or C18 spacers. Optionally, any one or more structural modifications may comprise one or more quantum dots (such as quantum dots of any size and/or composition and/or optical character). Optionally, any one or more structural modifications may comprise one or more nanoparticles (such as nanoparticles of any size and/or composition and/or optical character, such as gold nanoparticles of any size and/or composition). Optionally, any one or more structural modifications may comprise one or more solid supports (such as any bead or other solid support). Optionally, any two or more (or any larger number, such as any 3 or more, 5 or more, 10 or more, 20 or more, 50 or more, 100 or more, 500 or more, 1000 or more, 10,000 or more, or 100,000 or more) multimeric hybridization molecule (e.g. a multimeric barcode molecule), hybridization molecule, barcode molecule, barcoded oligonucleotide and/or adapter oligonucleotide may be (directly or indirectly) linked to and/or comprise any individual single structural modification (such as any single nanoparticle). Optionally, any one or more structural modifications may exhibit partially or predominantly anionic character; and/or any one or more structural modifications may exhibit partially or predominantly cationic character, any one or more structural modifications may exhibit zwitterionic character, any one or more structural modifications may exhibit partially or predominantly non-ionic character.

Optionally, any such structural modifications may be linked to and/or comprised within any multimeric hybridization molecule (e.g any multimeric barcode molecule), hybridization molecule, barcode molecule, barcoded oligonucleotide and/or adapter oligonucleotide by any direct or indirect attachment method and/or conjugation chemistry known in the art (including, but not limited to, any conjugation chemistry known in the art that may be employed to append/conjugate such structural modifications to an oligonucleotide after said oligonucleotide has been synthesised by a standard oligonucleotide process, e.g. phosphoramidite synthesis), such as by any covalent linkage (e.g. any amino-modification attachment chemistries, and/or any thiol-modification attachment chemistry, and/or any NHS-ester attachment chemistries, and/or any click-chemistry-related method (such as any copper (I)-catalysed azide-alkyne cycloaddition (CuAAC) reaction, a strain-promoted azide-alkyne cycloaddition (SPAAC) reaction, a strain-promoted alkyne-nitrone cycloaddition (SPANC) reaction, or an alkene and tetrazole photoclick reaction), and/or any non-covalent linkage (e.g. a protein-protein interaction or a streptavidin-biotin bond) and/or any nucleic acid hybridization, and/or by direct chemical synthesis of modified oligonucleotides (e.g. phosphoramidite oligonucleotide synthesis wherein any one or more structural modifications may be comprised within and/or linked to modified oligonucleotides employed during phosphoramidite synthesis of an oligonucleotide (such as phosphoramidite synthesis of a multimeric hybridization molecule, multimeric barcode molecule, hybridization molecule, barcode molecule, barcoded oligonucleotide and/or adapter oligonucleotide).

The multimeric hybridization molecules (e.g. a multimeric barcode molecules) may be linked by a support e.g. a macromolecule, solid support or semi-solid support. The sequences of the multimeric hybridization molecules (e.g. the multimeric barcode molecules) linked to each support may be known. The multimeric hybridization molecules (e.g. multimeric barcode molecules) may be linked to the support directly or indirectly (e.g. via a linker molecule). The multimeric hybridization molecules (e.g. multimeric barcode molecules) may be linked by being bound to the support and/or by being bound or annealed to linker molecules that are bound to the support. The multimeric hybridization molecules (e.g. multimeric barcode molecules) may be bound to the support (or to the linker molecules) by covalent linkage, non-covalent linkage (e.g. a protein-protein interaction or a streptavidin-biotin bond), electrostatic interactions, nucleic acid hybridization, via Van der Waals forces and/or hydrophobic interactions and/or via physisorption and/or chemisorption. The linker molecule may be a biopolymer (e.g. a nucleic acid molecule) or a synthetic polymer. The linker molecule may comprise one or more units of ethylene glycol and/or poly(ethylene) glycol (e.g. hexa-ethylene glycol or penta-ethylene glycol). The linker molecule may comprise one or more ethyl groups, such as a C3 (three-carbon) spacer, C6 spacer, C12 spacer, or C18 spacer. The linker molecule may comprise at least 2, at least 3, at least 4, at least 5, at least 10, or at least 20 or more sequential repeating units of any individual linker (such as a sequential linear series of at least 2, at least 5, or at least 10 C12 spacers or C18 spacers). The linker molecule may comprise a branched linker molecule, wherein 2 or more multimeric hybridization molecules (e.g. multimeric barcode molecules) are linked to a support by a single linker molecule.

The multimeric hybridization molecules (e.g. multimeric barcode molecules) may be linked by a macromolecule by being bound to the macromolecule and/or by being annealed to the macromolecule.

The multimeric hybridization molecules (e.g. multimeric barcode molecules) may be linked to the macromolecule directly or indirectly (e.g. via a linker molecule). The multimeric hybridization molecules (e.g. multimeric barcode molecules) may be linked by being bound to the macromolecule and/or by being bound or annealed to linker molecules that are bound to the macromolecule. The multimeric hybridization molecules (e.g. multimeric barcode molecules) may be bound to the macromolecule (or to the linker molecules) by covalent linkage, non-covalent linkage (e.g. a protein-protein interaction or a streptavidin-biotin bond), nucleic acid hybridization, electrostatic interactions, via Van der Waals forces and/or hydrophobic interactions and/or via physisorption and/or chemisorption. The linker molecule may be a biopolymer (e.g. a nucleic acid molecule) or a synthetic polymer. The linker molecule may comprise one or more units of ethylene glycol and/or poly(ethylene) glycol (e.g. hexa-ethylene glycol or penta-ethylene glycol). The linker molecule may comprise one or more ethyl groups, such as a C3 (three-carbon) spacer, C6 spacer, C12 spacer, or C18 spacer. The linker molecule may comprise at least 2, at least 3, at least 4, at least 5, at least 10, or at least 20 or more sequential repeating units of any individual linker (such as a sequential linear series of at least 2, at least 5, or at least 10 C12 spacers or C18 spacers). The linker molecule may comprise a branched linker molecule, wherein 2 or more multimeric hybridization molecules (e.g. multimeric barcode molecules) are linked to a support by a single linker molecule.

A multimeric hybridization molecule (e.g. a multimeric barcode molecule) may have many different designs. For example, in the case of a nucleic acid based multimeric hybridization molecule (e.g. a multimeric barcode molecule), the sequence of the oligonucleotide may vary the order and/or the presence of sequences of interest, modifications, linkers, branchers, attachment modifications and/or else. A multimeric hybridization molecule (e.g. a multimeric barcode molecule) may be (directly or indirectly) linked to and/or may comprise one or more structural modifications (that can be present anywhere), such as one or more natural or unnatural nucleotide and DNA modification (e.g. LNA, amino LNA, PNA, triazole backbone, amino backbone, 2′-O-methyl and/or 2′-O-methoxy-ethyl nucleosides, 2′-F and/or 2′-F-arabino nucleosides, phosphorothioates, modified bases (such as 2-aminopurine, tricyclic cytosines, 5-Bromo dU, etc.), fluorophores, intercalators, groove binders, etc.), such as one or more attachment modification (e.g. amino groups, carboxy groups, NHS esters, azido groups, alkyne groups, thiol groups, biotin/desthiobiotin, etc.), such as one or more brancher modification and/or linker molecules and/or linker moieties.

A multimeric hybridization molecule (e.g. a multimeric barcode molecule) may comprise at least 2, at least 3, at least 4, at least 5, at least 6, at least 7, at least 8, at least 9, at least 10, at least 20, at least 40, at least 60, at least 80, at least 100, at least 200 or more hybridization regions (i.e. the sequence to which an adapter region of a barcoded oligonucleotide anneals). The hybridization sequences of a single multimeric hybridization molecule (e.g. a multimeric barcode molecule) may be identical to each other or may be different to all or some of the others. The length of the hybridization regions may be at least 2, at least 3, at least 4, at least 5, at least 6, at least 7, at least 8, at least 9, at least 10, at least 20, at least 40, at least 60, at least 80, at least 100, at least 200 or more nucleotides long. The length of the hybridization regions may be identical for each hybridization region or different for all or some of the hybridization regions. None, some or all the hybridization regions may comprise modifications like natural or unnatural nucleotide/DNA/RNA/nucleic acid modifications aimed at modifying binding affinity, melting/annealing temperature, properties like fluorescence or more (e.g. LNA, amino LNA, PNA, triazole backbone, amino backbone, 2′-O-methyl and/or 2′-O-methoxy-ethyl nucleosides, 2′-F and/or 2′-F-arabino nucleosides, phosphorothioates, modified bases (such as 2-aminopurine, tricyclic cytosines, 5-Bromo dU, etc.), fluorophores, intercalators, groove binders, etc.). The modifications may be identical in all hybridization regions or different in all or some of the hybridization regions.

The hybridization regions may or may not be contiguous. A spacer may separate hybridization regions. A spacer may or may comprise a linker, a brancher, a cell-binding site, a capture site, an attachment site and/or a cleavage site. The type and/or the presence of spacers may be identical between multimeric hybridization molecules or different between multimeric hybridization molecules.

Hybridization regions may flank a spacer (i.e. hybridization regions may flank a linker, a brancher, a cell-binding site, a capture site, an attachment site and/or a cleavage site) and/or they may be part of one or more other annealing sequences and/or they may be part of one or more capture sites and/or they may overlap (partially or completely) with one or more capture sites and/or they may completely overlap with one or more of the cell-binding sites.

The hybridization regions may or may not be separated by a spacer composed of one or more linkers and/or by one or more branchers. The type and/or the presence and/or the number of the linkers and/or branchers may be identical throughout a multimeric hybridization molecule (e.g. a multimeric barcode molecule) or different in all or some of the positions that they occur in a multimeric hybridization molecule (e.g. a multimeric barcode molecule). The type and/or the presence and/or the number of the linkers and/or branchers may be identical throughout a spacer to different in all or some of the positions that they occur in a spacer. None, some or every linker molecule may be a biopolymer (e.g. a nucleic acid molecule, peptide, etc.) or a synthetic polymer. None, some or every linker molecule may comprise one or more units of ethylene glycol and/or poly(ethylene) glycol (e.g. hexa-ethylene glycol or penta-ethylene glycol). None, some or every linker molecule may comprise one or more ethyl groups, such as one or more C3 (three-carbon) spacers, C6 spacers, C12 spacers, or C18 spacers. None, some or every linker molecule may be made of any other chemistry or polymer (e.g. peptide based polymers, nucleic acid based polymers like for example PNA, any synthetic polymer, etc.).

None, some or every spacer in the multimeric hybridization molecule (e.g. multimeric barcode molecule) may have one or more brancher modification and/or linker molecules and/or linker moieties, such as one or more units of ethylene glycol and/or poly(ethylene) glycol (e.g. hexa-ethylene glycol and/or penta-ethylene glycol and/or PEG 500, PEG 1000, PEG 2000, PEG 4000, PEG 5000, PEG 10,000, and/or PEG 20,000; optionally, any such PEG moiety may have any degree of dispersity in size/molecular mass, i.e. any degree of monodispersity or polydispersity), and/or any one or more C3 (three-carbon) spacers, C6 spacers, C12 spacers, or C18 spacers.

Optionally, any number of one or more such structural modifications (e.g. linker molecules and/or linker moieties) may be added to any individual spacer(s), such as at least 2 linker molecules and/or linker moieties, at least 3 linker molecules and/or linker moieties, at least 4 linker molecules and/or linker moieties, at least 5 linker molecules and/or linker moieties, at least 6 linker molecules and/or linker moieties, at least 8 linker molecules and/or linker moieties, at least linker molecules and/or linker moieties, at least 15 linker molecules and/or linker moieties, at least 20 linker molecules and/or linker moieties, at least 30 linker molecules and/or linker moieties, at least 40 linker molecules and/or linker moieties, at least 50 linker molecules and/or linker moieties, or at least 100 or more linker molecules and/or linker moieties. Optionally, any such linker molecules and/or linker moieties may comprise branched linker molecules or linker moieties (such as a branched linker molecule comprising two or more ethyl groups, such as two or more spacer moieties, such as two or more C3 (three-carbon) spacers, and/or C6 spacers, and/or C12 spacers, and/or C18 spacers. Optionally, any such linker molecules and/or linker moieties may comprise sequentially-connected (i.e. linear) linker molecules or linker moieties (such as a sequential repeating units of a linker molecule comprising two or more ethyl groups in linear series, such as two or more spacer moieties, such as two or more C3 (three-carbon) spacers, and/or C6 spacers, and/or C12 spacers, and/or C18 spacers. Optionally, any one or more structural modifications may exhibit partially or predominantly anionic character; and/or any one or more structural modifications may exhibit partially or predominantly cationic character, any one or more structural modifications may exhibit zwitterionic character, any one or more structural modifications may exhibit partially or predominantly non-ionic character.

A multimeric hybridization molecule (e.g. a multimeric barcode molecule) may or may not contain one or more attachment sites. Attachment sites may be flanking one or more annealing sequences and/or flanking one or more spacers and/or flanking one or more cleavage sites and/or flanking one or more cell binding sites and/or flanking one or more capture sites and/or they may be part of one or more annealing sequences and/or they may be part of one or more spacers and/or they may be part of one or more cleavage sites and/or they may be part of one or more cell binding sites and/or they may be part of one or more capture sites. In the case of an oligonucleotide based multimeric hybridization molecule (e.g. multimeric barcode molecule), the attachment site(s) may be at the 3′-end(s) of the sequence and/or at the 5′-end(s) of the sequence and/or anywhere in-between the two or more ends. Attachment sites may work via covalent linkage, non-covalent linkage (e.g. a protein-protein interaction or a streptavidin-biotin bond), electrostatic interactions, nucleic acid hybridization, Van der Waals forces and/or hydrophobic interactions. For example, attachment sites may be composed in a way to generate one or more covalent linkage(s) (or bond(s)) (e.g. by a covalent bond such as a bond generated by any amino-modification attachment chemistry, and/or any carboxy-modification attachment chemistry, and/or any thiol-modification attachment chemistry, and/or any NHS-ester attachment chemistry, and/or any click-chemistry-related method, such as any copper (I)-catalysed azide-alkyne cycloaddition (CuAAC) reaction, a strain-promoted azide-alkyne cycloaddition (SPAAC) reaction, a strain-promoted alkyne-nitrone cycloaddition (SPANC) reaction, or an alkene and tetrazole photoclick reaction), one or more non-covalent linkages (or bond(s)) (e.g. a protein-protein interaction or a streptavidin-biotin linkage e.g. the attachment site may have one or more biotins/desthiobiotins) and/or a nucleic acid hybridization linkage.

A multimeric hybridization molecule (e.g. a multimeric barcode molecule) may or may not contain one or more cleavage sites. Cleavage sites may be flanking one or more annealing sequences and/or flanking one or more spacers and/or flanking one or more attachment sites and/or flanking one or more cell binding sites and/or flanking one or more capture sites and/or they may be part of one or more annealing sequences and/or they may be part of one or more spacers and/or they may be part of one or more attachment sites and/or they may be part of one or more cell binding sites and/or they might be part of one or more capture sites. Cleavage sites can be made according to any chemistry or biochemistry that allows cleavage and/or cutting and/or digestion at and/or from a specific location. For example, a cleavage site may be composed by one or more modifications with any kind of reversible/cleavable linker chemistry (e.g. disulfides, photocleavable linkers, peptides to be cleaved with a peptidase, etc.) that uses any chemical, physico-chemical, biochemical and/or enzymatic process and/or change to achieve the cleavage. A cleavage site may also be composed by one or more modified nucleotides that can be cleaved via any chemical, physico-chemical, biochemical and/or enzymatic process and/or change (e.g. U can be cleaved by enzymes like USER®, 8oxoG can be cleaved by enzymes like FpG, etc.). A cleavage site may also for example be composed by one or more specific sequences that might be cut by an enzyme like a restriction nuclease.

A multimeric hybridization molecule (e.g. a multimeric barcode molecule) may or may not comprise a cell-binding site (e.g. a cell-binding sequence such as a cell-binding oligonucleotide). Cell-binding sites may be flanking one or more hybridization regions and/or flanking one or more spacers and/or flanking one or more attachment sites and/or flanking one or more cleavage sites and/or flanking one or more capture sites and/or they may be part of one or more hybridization regions and/or they may be part of one or more spacers and/or they may be part of one or more attachment sites and/or they may be part of one or more cleavage sites and/or they might be part of one or more capture sites and/or they may overlap (partially or completely) with one or more of the hybridization regions and/or they may overlap (partially or completely) with one or more of the capture sites. A cell-binding site may comprise a cell-binding moiety. A cell-binding site may be located anywhere on the multimeric hybridization molecule (e.g. multimeric barcode molecule). For example, it may be at the 3′-end(s) and/or at the 5′-end(s) and/or anywhere in-between the ends.

A cell-binding site (e.g. a cell-binding sequence) may be linked to a multimeric hybridization molecule (e.g. a multimeric barcode molecule). For example, the linkage may be a covalent linkage, a non-covalent linkage (e.g. a protein-protein interaction or a streptavidin-biotin bond), nucleic acid hybridization, electrostatic interactions and/or via Van der Waals forces and/or hydrophobic interactions. A linker may or may not be present, the linker molecule may be a biopolymer (e.g. a nucleic acid molecule) or a synthetic polymer. The linker molecule may comprise one or more units of ethylene glycol and/or poly(ethylene) glycol (e.g. hexa-ethylene glycol or penta-ethylene glycol). The linker molecule may comprise one or more ethyl groups, such as a C3 (three-carbon) spacer, C6 spacer, C12 spacer, or C18 spacer. The linker molecule may comprise at least 2, at least 3, at least 4, at least 5, at least 10, or at least 20 or more sequential repeating units of any individual linker (such as a sequential linear series of at least 2, at least 5, or at least 10 C12 spacers or C18 spacers). The linker molecule may comprise a branched linker molecule, wherein 2 or more cell binding sites are linked to a multimeric hybridization molecule (e.g. a multimeric barcode molecule) by a single linker molecule.

A cell-binding site (e.g. a cell-binding sequence) may be linked to a multimeric hybridization molecule (e.g. a multimeric barcode molecule) via one or more covalent linkage(s) (or bond(s)) (e.g. by a covalent bond such as a bond generated by any amino-modification attachment chemistry, and/or any carboxy-modification attachment chemistry, and/or any thiol-modification attachment chemistry, and/or any NHS-ester attachment chemistry, and/or any click-chemistry-related method, such as any copper (I)-catalysed azide-alkyne cycloaddition (CuAAC) reaction, a strain-promoted azide-alkyne cycloaddition (SPAAC) reaction, a strain-promoted alkyne-nitrone cycloaddition (SPANC) reaction, or an alkene and tetrazole photoclick reaction), one or more non-covalent linkages (or bond(s)) (e.g. a protein-protein interaction or a streptavidin-biotin linkage e.g. a support may comprise a streptavidin domain and a multimeric hybridization molecule (e.g. a multimeric barcode molecule) may comprise a biotin moiety), electrostatic interactions (e.g. a polycationic polymer like poly-lysine interacting with a polyanionic polymer like DNA) or a nucleic acid hybridization linkage. Any one or more linker molecule may be a biopolymer (e.g. a nucleic acid molecule, peptide, etc.) or a synthetic polymer. Any one or more linker molecule may comprise one or more units of ethylene glycol and/or poly(ethylene) glycol (e.g. hexa-ethylene glycol or penta-ethylene glycol). Any one or more linker molecule may comprise one or more ethyl groups, such as one or more C3 (three-carbon) spacers, C6 spacers, C12 spacers, or C18 spacers. Any one or more linker molecule may be made of any other chemistry or polymer (e.g. peptide based polymers, nucleic acid based polymers like for example PNA, any synthetic polymer, etc.).

A multimeric hybridization molecule (e.g. a multimeric barcode molecule) may or may not contain one or more capture sites. Capture sites may be flanking one or more hybridization regions and/or flanking one or more spacers and/or flanking one or more attachment sites and/or flanking one or more cleavage sites and/or flanking one or more cell-binding sites and/or they may be part of one or more hybridization regions and/or they may be part of one or more spacers and/or they may be part of one or more attachment sites and/or they may be part of one or more cleavage sites and/or they might be part of one or more cell-binding sites and/or they may overlap (partially or completely) with one or more of the hybridization regions and/or they may overlap (partially or completely) with one or more of the cell-binding sites. A capture site may contain a molecule with specific affinity to the target that needs to be barcoded by a multimeric barcoding reagent. For example, a capture site could be a poly-T oligonucleotide complementary to the poly-A tail of mRNA or any other sequence complementary to any target sequence of interest. A capture site may be located anywhere on the multimeric hybridization molecule (e.g. multimeric barcode molecule). For example, it could be at the 3′-end(s) and/or at the 5′-end(s) and/or anywhere in-between the ends.

Optionally, a multimeric hybridization molecule (e.g. a multimeric barcode molecule) may comprise a modification that can change the orientation of the multimeric hybridization molecule (or multimeric barcode molecule). For example, a nucleic acid based multimeric hybridization molecule (e.g. a multimeric barcode molecule) may end up having only 3′-ends or only 5′-ends. This could be achieved for example by using asymmetrical branchers and/or via conjugation of two or more oligonucleotides by linking two identical ends (i.e. a 3′-end with a 3′-end or a 5′-end with a 5′-end).

A multimeric hybridization molecule (e.g. a multimeric barcode molecule) may be designed to have specific secondary structures in different locations, these structures may be selected to be tuneable and/or to have a specific stability (i.e. with specific annealing and/or melting temperature, being formed or dissolved at specific temperatures). For example, the multimeric hybridization molecule (e.g. multimeric barcode molecule) may be designed to close on itself (e.g. via self-hybridization in a hairpin like fashion) after a certain step or at certain temperatures.

The size of a multimeric hybridization molecule (e.g. multimeric barcode molecule) may vary, for example the molecular weight could be at least 1 kDa, at least 5 kDa, at least 10 kDa, at least 50 kDa, at least 100 kDa, at least 500 kDa, at least 1000 kDa, at least 5000 kDa, at least 10000 kDa, at least 50000 kDa, at least 100000 kDa or more.

Multimeric hybridization molecules (e.g. multimeric barcode molecules) may be produces via multiple methodologies that a person having ordinary skill in the art can imagine. For example, short (below 1000 nucleotides) chemically modified oligonucleotides may be produced through the standard phosphoramidite oligonucleotide synthesis, longer sequences may be then composed by ligating and/or extending these shorter oligonucleotides. Different ligation and/or extension methodologies may be used. For example, any direct or indirect attachment method and/or ligation method and/or extension method and/or conjugation chemistry known in the art, such as by any chemically-formed covalent linkage (e.g. any amino-modification attachment chemistries, and/or any thiol-modification attachment chemistry, and/or any NHS-ester attachment chemistries, and/or any click-chemistry-related method (such as any copper (I)-catalysed azide-alkyne cycloaddition (CuAAC) reaction, a strain-promoted azide-alkyne cycloaddition (SPAAC) reaction, a strain-promoted alkyne-nitrone cycloaddition (SPANC) reaction, or an alkene and tetrazole photoclick reaction), and/or any enzymatically-formed covalent linkage (e.g. via templated and/or non-templated ligation with a ligase, via terminal transferases and polymerase extension, etc.), and/or any non-covalent linkage (e.g. a protein-protein interaction or a streptavidin-biotin bond) and/or any nucleic acid hybridization.

A long multimeric hybridization molecule (e.g. a long multimeric barcode molecule) may for example be produced by generating a long molecule comprising the same sequence motif repeating throughout via the RCA extension of a circular template. The addition of a specific sequence or modification at the end of the long RCA product may be achieved, for example, through the ligation of a second oligo via a ligase and/or the use of a terminal transferase to add a modified nucleotide (this nucleotide might, for example, contain modifications for chemical conjugation via any conjugation chemistry known in the art, e.g. an amino group for peptide coupling, an alkyne for CuAAC ligation, etc.) and/or the use of a terminal transferase with subsequent annealing and enzymatic/polymerase copying/extension.

During the production of a multimeric hybridization molecule (e.g. a multimeric barcode molecule) the elongation may be achieved by using orthogonal chemistries to avoid cyclization of each segment of the multimeric hybridization molecule (e.g. the multimeric barcode molecule) being formed. For example, one segment could have an amino group at the 3′-end for a peptide coupling and an alkyne group at the 5′-end for a CuAAC coupling. The preparation of a multimeric hybridization molecule (e.g. a multimteric barcode molecule) via elongation may be performed directly on a support. The advantage of this is that removing unconjugated oligonucleotides can be easily performed via washes with a number of solutions and/or buffers and/or solvents. In this scenario, the construction of a multimeric hybridization molecule (e.g. a multimeric barcode molecule) may be performed through the alternation of two orthogonal conjugation chemistries that could be chosen from multiple options available (e.g. one segment could have an amino group at the 3′-end for a peptide coupling and an alkyne group at the 5′-end for a CuAAC coupling). The production of the multimeric hybridization molecule (e.g. the multimeric barcode molecule) on a support may be automated.

The support may be a solid support or a semi-solid support. The support may comprise a non-planar and/or a planar surface. The support may be a slide e.g. a glass slide. The slide may be a flow cell for sequencing. If the support is a slide, the first and second hybridization molecules may be immobilized in a discrete region on the slide. Optionally, the hybridization molecules of each multimeric barcoding reagent in a library are immobilized in a different discrete region on the slide to the hybridization molecules of the other multimeric barcoding reagents in the library. The support may be a plate comprising wells, optionally wherein the first and second hybridization molecules are immobilized in the same well. Optionally, the hybridization molecules of each multimeric barcoding reagent in library are immobilized in a different well of the plate to the hybridization molecules of the other multimeric barcoding reagents in the library.

Preferably, the support is a bead (e.g. a gel bead). The bead may be a polymer-based bead, an agarose bead, a silica bead, a styrofoam/polystyrene bead, a dextran bead, a polylactic acid bead, a polyvinyl alcohol bead, a gel bead (such as those available from 10× Genomics®), an antibody conjugated bead, an oligo-dT conjugated bead, a streptavidin bead or a magnetic bead (e.g. a superparamagnetic bead). The bead may be a microbead (e.g. a magnetic microbead). The bead may be of any size and/or molecular structure. For example, the bead may be 10 nanometres to 200 microns in diameter, 10 nanometres to 100 microns in diameter, 100 nanometres to 10 microns in diameter, 1 micron to 5 microns in diameter or 10 microns to 50 microns in diameter. Optionally, the bead is approximately 10 nanometres in diameter, approximately 100 nanometres in diameter, approximately 1 micron in diameter, approximately 10 microns in diameter or approximately 100 microns in diameter. The bead may be solid, or alternatively the bead may be hollow or partially hollow or porous. Beads of certain sizes may be most preferable for certain barcoding methods. For example, beads less than 35.0 microns, less than 5.0 microns, or less than 1.0 micron, may be most useful for barcoding nucleic acid targets within individual cells. Preferably, the hybridization molecules of each multimeric barcoding reagent in a library are linked together on a different bead to hybridization molecules of the other multimeric barcoding reagents in the library.

The support may be functionalised to enable attachment of two or more hybridization molecules. This functionalisation may be enabled through the addition of chemical moieties (e.g. carboxylated groups, alkynes, azides, acrylate groups, amino groups, sulphate groups, tosyl groups, epoxy groups, thiols, maleimides, iodoacetyl groups, Au for thiol-Au based approaches or succinimide/NHS ester groups), and/or protein-based moieties (e.g. streptavidin, avidin, or protein G) to the support. The hybridization molecules may be attached to the moieties directly or indirectly (e.g. via a linker molecule).

Barcoded oligonucleotides and/or multimeric hybridization molecules may be linked to a support by amine-carboxylic acid/NHS-ester peptide coupling, azide-alkyne click chemistry (e.g. CuAAC or SPAAC), non-covalent interaction (e.g. streptavidin-biotin or thiol-based approaches such as thiol-maleimide), disulfide and thiol-Au interactions.

Functionalised supports (e.g. beads) may be brought into contact with a solution of hybridization molecules under conditions which promote the attachment of two or more hybridization molecules to each bead in the solution (generating multimeric barcoding reagents).

In a library of multimeric barcoding reagents, the hybridization molecules of each multimeric barcoding reagent in a library may be linked together on a different support to the hybridization molecules of the other multimeric barcoding reagents in the library.

Optionally, the hybridization molecules are attached to the beads by covalent linkage, non-covalent linkage (e.g. a streptavidin-biotin bond) or nucleic acid hybridization.

A cell binding moiety may be can be linked to the support covalently, non-covalently, electrostatically, via Van der Waals forces and/or hydrophobic interactions, via physisorption and/or chemisorption, either directly or via a linker. For example, the linker may be a nucleic acid based linker. The cell binding moiety may be linked to an oligonucleotide hybridised to a multimeric hybridization molecule (e.g. a multimeric barcode molecule) and/or it may be part of the multimeric hybridization molecule (e.g. the multimeric barcode molecule).

Optionally, the cell binding moiety may be formed by an oligonucleotide with a cell binding modification attached to the 3′-end of the oligonucleotide, with such oligonucleotide in turn hybridizing to a multimeric hybridization molecule (e.g. a multimeric barcode molecule). Example cell binding modifications may include fatty acid based modifications (e.g. palmitate based, oleate based, stearate based, etc.) (Eurogentec), cholesterol based modifications (Eurogentec), phospholipid based modifications (e.g. 1,2-distearoyl-sn-glycero-3-phosphocholine (DSPC) based, 1,2-dioleoyl-sn-glycero-3-phosphoethanolamine (DOPE) based, 1,2-distearoyl-sn-glycero-3-phosphorylethanolamine (DSPE) based, etc.) (Eurogentec and IDT), pyrene based modifications (IDT), retinoid acid based modifications (Eurogentec) and tocopherol based modifications (Eurogentec).

In some instances the cell binding moiety of choice may allow to obtain a cell-specific binding. For example, the use of antibodies and/or aptamers would allow to target specific cell types with high levels of precision. Multimeric barcoding reagents may or may not contain one or more cell-specific cell-binding moieties. The cell-specific cell-binding moieties may be anything from all identical to all different from each other. A cell-specific cell-binding moiety may be located anywhere on the multimeric barcoding reagent, for example the cell-specific cell-binding moiety may be located on a support and/or on a multimeric hybridization molecule (e.g. a multimeric barcode molecule) and/or on a barcoded oligonucleotide. A library of multimeric barcoding reagents may contain anywhere from the same cell-specific cell-binding moiety on each multimeric barcoding reagent to having a different cell-specific cell-binding moiety on each multimeric barcoding reagent. For example, a multimeric barcoding reagent and/or a library of multimeric barcoding reagents may target one specific cell type or it may target at least 2, at least 3, at least 4, at least 5, at least 10, at least 20, at least 50, at least 100, at least 500 or more cell types.

Optionally, a multimeric barcoding reagent may contain an antibody specific for a protein on the surface of a specific cell type allowing to achieve binding to only that type of cell in the presence of numerous other ones (e.g. an antibody specific for T cells in the context of a blood sample). Optionally, a multimeric barcoding reagent may contain multiple different antibodies all specific for the same cell type. Optionally, a multimeric barcoding reagent may contain multiple different antibodies specific for different cell types, for example it may contain two types of antibodies, one specific for B cells and one specific for T cells, allowing so to target specifically immune cells in a sample (e.g. a blood sample).

The multimeric barcoding reagent may comprise: at least 2, at least 3, at least 4, at least 5, at least 10, at least 20, at least 25, at least 50, at least 75, at least 100, at least 200, at least 500, at least 1000, at least 5000, at least 10⁴, at least 10⁵, at least 10⁶, at least 10⁷, at least 10⁸, at least 10⁹ or at least 10¹⁰ hybridization molecules linked together, wherein each hybridization molecule is as defined herein; and a barcoded oligonucleotide annealed to each hybridization molecule, wherein each barcoded oligonucleotide is as defined herein. Preferably, the multimeric barcoding reagent comprises at least 5 hybridization molecules linked together, wherein each hybridization molecule is as defined herein; and a barcoded oligonucleotide annealed to each hybridization molecule, wherein each barcoded oligonucleotide is as defined herein.

The multimeric barcoding reagent may comprise: at least 2, at least 3, at least 4, at least 5, at least 10, at least 20, at least 25, at least 50, at least 75, at least 100, at least 200, at least 500, at least 1000, at least 5000, at least 10⁴, at least 10⁵, at least 10⁶, at least 10⁷, at least 10⁸, at least 10⁹ or at least 10¹⁰ unique or different hybridization molecules linked together, wherein each hybridization molecule is as defined herein; and a barcoded oligonucleotide annealed to each hybridization molecule, wherein each barcoded oligonucleotide is as defined herein. Preferably, the multimeric barcoding reagent comprises at least 5 unique or different hybridization molecules linked together, wherein each hybridization molecule is as defined herein; and a barcoded oligonucleotide annealed to each hybridization molecule, wherein each barcoded oligonucleotide is as defined herein.

The multimeric hybridization molecule may be a multimeric barcode molecule, wherein the first hybridization molecule is a first barcode molecule and the second hybridization molecule is a second barcode molecule. A multimeric barcoding reagent may comprise: first and second barcode molecules linked together (i.e. a multimeric barcode molecule), wherein each of the barcode molecules comprises a nucleic acid sequence comprising a barcode region; and first and second barcoded oligonucleotides, wherein the first barcoded oligonucleotide is annealed to the barcode region of the first barcode molecule, and wherein the second barcoded oligonucleotide is annealed to the barcode region of the second barcode molecule.

The barcoded oligonucleotides of a multimeric barcoding reagent may comprise: a first barcoded oligonucleotide comprising, optionally in the 5′ to 3′ direction, a barcode region, and a target region capable of annealing or ligating to a first sub-sequence of the target nucleic acid; and a second barcoded oligonucleotide comprising, optionally in the 5′ to 3′ direction, a barcode region, and a target region capable of annealing or ligating to a second sub-sequence of the target nucleic acid.

The barcoded oligonucleotides of a multimeric barcoding reagent may comprise: a first barcoded oligonucleotide comprising a barcode region, and a target region capable of ligating to a first sub-sequence of the target nucleic acid; and a second barcoded oligonucleotide comprising a barcode region, and a target region capable of ligating to a second sub-sequence of the target nucleic acid.

The barcoded oligonucleotides of a multimeric barcoding reagent may comprise: a first barcoded oligonucleotide comprising, in the 5′ to 3′ direction, a barcode region, and a target region capable of annealing to a first sub-sequence of the target nucleic acid; and a second barcoded oligonucleotide comprising, in the 5′ to 3′ direction, a barcode region, and a target region capable of annealing to a second sub-sequence of the target nucleic acid.

A multimeric barcoding reagent, multimeric hybridization molecule, multimeric barcode molecule, hybridization molecule, barcode molecule, barcoded oligonucleotide and/or adapter oligonucleotide may comprise one or more capture sites. Capture sites may be identical in each multimeric barcoding reagent, multimeric hybridization molecule, multimeric barcode molecule, hybridization molecule, barcode molecule, barcoded oligonucleotide and/or adapter oligonucleotide, or they may be different to all or some of the other capture agents. A capture site may comprise a molecule with specific affinity to the target that needs to be barcoded by a multimeric barcoding reagent. A capture site may be a target region e.g. a poly-T oligonucleotide complementary to the poly-A tail of mRNA or any other sequence complementary to any target sequence of interest. A capture site may be a target-specific macromolecule (e.g. an antibody and/or an aptamer). A capture site may be located anywhere on the multimeric barcoding reagent, for example a capture site may be located on a support and/or on a multimeric hybridization molecule (e.g. on a multimeric barcode molecule) and/or on a barcoded oligonucleotide. Preferably, a capture site is located on the more exterior part of the multimeric barcoding reagent to facilitate interaction with its target. The capture site (e.g. a target region) may be at the 5′ or 3′ end of a barcoded oligonucleotide. A capture site may be linked to a multimeric barcoding reagent (and therefore to any of its parts, e.g. the support and/or a multimeric hybridization molecule (e.g. the multimeric barcode molecule), etc.) by a covalent linkage, a non-covalent linkage (e.g. a protein-protein interaction or a streptavidin-biotin bond), nucleic acid hybridization, electrostatic interactions, via Van der Waals forces and/or hydrophobic interactions and/or via physisorption and/or chemisorption. A linker may or may not be present, the linker molecule may be a biopolymer (e.g. a nucleic acid molecule) or a synthetic polymer. The linker molecule may comprise one or more units of ethylene glycol and/or poly(ethylene) glycol (e.g. hexa-ethylene glycol or penta-ethylene glycol). The linker molecule may comprise one or more ethyl groups, such as a C3 (three-carbon) spacer, C6 spacer, C12 spacer, or C18 spacer. The linker molecule may comprise at least 2, at least 3, at least 4, at least 5, at least 10, or at least 20 or more sequential repeating units of any individual linker (such as a sequential linear series of at least 2, at least 5, or at least 10 C12 spacers or C18 spacers). The linker molecule may comprise a branched linker molecule, wherein 2 or more capture sites are linked to a multimeric barcoding reagent (or a multimeric hybridization molecule, multimeric barcode molecule, hybridization molecule, barcode molecule, barcoded oligonucleotide and/or adapter oligonucleotide) by a single linker molecule.

A capture site may be attached (or linked) to a multimeric barcoding reagent, multimeric hybridization molecule, multimeric barcode molecule, hybridization molecule, barcode molecule, barcoded oligonucleotide and/or adapter oligonucleotide by a covalent linkage or by a non-covalent linkage (e.g. a protein-protein interaction or a streptavidin-biotin bond), nucleic acid hybridization, electrostatic interactions, via Van der Waals forces and/or hydrophobic interactions and/or via physisorption and/or chemisorption.

A capture site may be attached (or linked) to a multimeric barcoding reagent, multimeric hybridization molecule, multimeric barcode molecule, hybridization molecule, barcode molecule, barcoded oligonucleotide and/or adapter oligonucleotide by a linker molecule. Optionally, said linker may be a flexible linker. The linker molecule may be a biopolymer (e.g. a nucleic acid molecule or peptide) or a synthetic polymer. The linker molecule may be a peptide-based polymers or a nucleic acid based polymer (e.g. PNA). The linker molecule may comprise one or more units of ethylene glycol and/or poly(ethylene) glycol (e.g. hexa-ethylene glycol or penta-ethylene glycol). The linker molecule may comprise one or more ethyl groups, such as a C3 (three-carbon) spacer, C6 spacer, C12 spacer, or C18 spacer. The linker molecule may comprise at least 2, at least 3, at least 4, at least 5, at least 10, or at least 20 or more sequential repeating units of any individual linker (such as a sequential linear series of at least 2, at least 5, or at least 10 C12 spacers or C18 spacers). The linker molecule may comprise a branched linker molecule, wherein 2 or more capture sites are linked to a multimeric barcoding reagent (or a multimeric hybridization molecule, multimeric barcode molecule, hybridization molecule, barcode molecule, barcoded oligonucleotide and/or adapter oligonucleotide) by a single linker molecule.

A capture site may be attached (or linked) to a multimeric barcoding reagent, multimeric hybridization molecule, multimeric barcode molecule, hybridization molecule, barcode molecule, barcoded oligonucleotide and/or adapter oligonucleotide by one or more covalent linkage(s) (or bond(s)) (e.g. by a covalent bond such as a bond generated by any amino-modification attachment chemistry, and/or any carboxy-modification attachment chemistry, and/or any thiol-modification attachment chemistry, and/or any NHS-ester attachment chemistry, and/or any click-chemistry-related method, such as any copper (I)-catalysed azide-alkyne cycloaddition (CuAAC) reaction, a strain-promoted azide-alkyne cycloaddition (SPAAC) reaction, a strain-promoted alkyne-nitrone cycloaddition (SPANC) reaction, or an alkene and tetrazole photoclick reaction), one or more non-covalent linkages (or bond(s)) (e.g. a protein-protein interaction or a streptavidin-biotin linkage e.g. a support may comprise a streptavidin domain and a multimeric hybridization molecule (e.g. a multimeric barcode molecule) may comprise a biotin moiety), physisorption and/or chemisorption (e.g. thiol-Au interactions), electrostatic interactions (e.g. a polycationic polymer like poly-lysine interacting with a polyanionic polymer like DNA) or a nucleic acid hybridization linkage.

### 2. GENERAL PROPERTIES OF BARCODED OLIGONUCLEOTIDES

A barcoded oligonucleotide comprises a barcode region. The barcoded oligonucleotides may comprise, optionally in the 5′ to 3′ direction, a barcode region and a target region. The target region is capable of annealing or ligating to a sub-sequence of the target nucleic acid. Alternatively, a barcoded oligonucleotide may consist essentially of or consist of a barcode region.

The 5′ end of a barcoded oligonucleotide may be phosphorylated. This may enable the 5′ end of the barcoded oligonucleotide to be ligated to the 3′ end of a target nucleic acid. Alternatively, the 5′ end of a barcoded oligonucleotide may not be phosphorylated.

A barcoded oligonucleotide may be a single-stranded nucleic acid molecule (e.g. single-stranded DNA). A barcoded oligonucleotide may comprise one or more double-stranded regions. A barcoded oligonucleotide may be a double-stranded nucleic acid molecule (e.g. double-stranded DNA).

The barcoded oligonucleotides may comprise or consist of deoxyribonucleotides. One or more of the deoxyribonucleotides may be a modified deoxyribonucleotide (e.g. a deoxyribonucleotide modified with a biotin moiety or a deoxyuracil nucleotide). The barcoded oligonucleodides may comprise one or more degenerate nucleotides or sequences. The barcoded oligonucleotides may not comprise any degenerate nucleotides or sequences.

The barcode regions of each barcoded oligonucleotide may comprise different sequences. Each barcode region may comprise a sequence that identifies the multimeric barcoding reagent. For example, this sequence may be a constant region shared by all barcode regions of a single multimeric barcoding reagent. The barcode region of each barcoded oligonucleotide may contain a unique sequence which is not present in other barcoded oligonucleotides, and may thus serve to uniquely identify each barcoded oligonucleotide. Each barcode region may comprise at least 5, at least 10, at least 15, at least 20, at least 25, at least 50 or at least 100 nucleotides. Preferably, each barcode region comprises at least 5 nucleotides. Preferably each barcode region comprises deoxyribonucleotides, optionally all of the nucleotides in a barcode region are deoxyribonucleotides. One or more of the deoxyribonucleotides may be a modified deoxyribonucleotide (e.g. a deoxyribonucleotide modified with a biotin moiety or a deoxyuracil nucleotide). The barcode regions may comprise one or more degenerate nucleotides or sequences. The barcode regions may not comprise any degenerate nucleotides or sequences.

The target regions of each barcoded oligonucleotide may comprise different sequences. Each target region may comprise a sequence capable of annealing to only a single sub-sequence of a target nucleic acid within a sample of nucleic acids (i.e. a target specific sequence). Each target region may comprise one or more random, or one or more degenerate, sequences to enable the target region to anneal to more than one sub-sequence of a target nucleic acid. Each target region may comprise at least 5, at least 10, at least 15, at least 20, at least 25, at least 50 or at least 100 nucleotides. Preferably, each target region comprises at least 5 nucleotides. Each target region may comprise 5 to 100 nucleotides, 5 to 10 nucleotides, 10 to 20 nucleotides, 20 to 30 nucleotides, 30 to 50 nucleotides, 50 to 100 nucleotides, 10 to 90 nucleotides, 20 to 80 nucleotides, 30 to 70 nucleotides or 50 to 60 nucleotides. Preferably, each target region comprises 30 to 70 nucleotides. Preferably each target region comprises deoxyribonucleotides, optionally all of the nucleotides in a target region are deoxyribonucleotides. One or more of the deoxyribonucleotides may be a modified deoxyribonucleotide (e.g. a deoxyribonucleotide modified with a biotin moiety or a deoxyuracil nucleotide). Each target region may comprise one or more universal bases (e.g. inosine), one or modified nucleotides and/or one or more nucleotide analogues.

The target regions may be used to anneal the barcoded oligonucleotides to sub-sequences of target nucleic acids, and then may be used as primers for a primer-extension reaction or an amplification reaction e.g. a polymerase chain reaction. Alternatively, the target regions may be used to ligate the barcoded oligonucleotides to sub-sequences of target nucleic acids. The target region may be at the 5′ end of a barcoded oligonucleotide. Such a target region may be phosphorylated. This may enable the 5′ end of the target region to be ligated to the 3′ end of a sub-sequence of a target nucleic acid.

The barcoded oligonucleotides may further comprise one or more adapter region(s). An adapter region may be between the barcode region and the target region. A barcoded oligonucleotide may, for example, comprise an adapter region 5′ of a barcode region (a 5′ adapter region) and/or an adapter region 3′ of the barcode region (a 3′ adapter region). Optionally, the barcoded oligonucleotides comprise, in the 5′ to 3′ direction, a barcode region, an adapter region and a target region.

The adapter region(s) of the barcoded oligonucleotides may comprise a sequence complementary to an adapter region of a multimeric barcode molecule or a sequence complementary to a hybridization region of a multimeric hybridization molecule. The adapter region(s) of the barcoded oligonucleotides may enable the barcoded oligonucleotides to be linked to a macromolecule or support (e.g. a bead). The adapter region(s) may be used for manipulating, purifying, retrieving, amplifying, or detecting barcoded oligonucleotides and/or target nucleic acids to which they may anneal or ligate.

The adapter region of each barcoded oligonucleotide may comprise a constant region. Optionally, all adapter regions of barcoded oligonucleotides of each multimeric barcoding reagent are substantially identical. The adapter region may comprise at least 1, at least 2, at least 3, at least 4, at least 5, at least 6, at least 8, at least 10, at least 15, at least 20, at least 25, at least 50, at least 100, or at least 250 nucleotides. Preferably, the adapter region comprises at least 4 nucleotides. Preferably each adapter region comprises deoxyribonucleotides, optionally all of the nucleotides in an adapter region are deoxyribonucleotides. One or more of the deoxyribonucleotides may be a modified deoxyribonucleotide (e.g. a deoxyribonucleotide modified with a biotin moiety or a deoxyuracil nucleotide). Each adapter region may comprise one or more universal bases (e.g. inosine), one or modified nucleotides and/or one or more nucleotide analogues.

A barcoded oligonucleotide may be linked to an affinity moiety directly or indirectly (e.g. via one or more linker molecules). A barcoded oligonucleotide may be linked to an affinity moiety via a linker molecule, wherein said linker molecule is appended to and/or linked to and/or bound to (covalently or non-covalently) both at least one affinity moiety, and at least one barcoded oligonucleotide. A barcoded oligonucleotide may be linked to any affinity moiety by one or more covalent linkage(s) (or bond(s)) (e.g. by a covalent bond such as a bond created by the LighteningLink® antibody labelling kit, Innova Biosciences), one or more non-covalent linkages (or bond(s)) (e.g. a protein-protein interaction or a streptavidin-biotin linkage e.g. an affinity moiety may comprise a streptavidin domain and a barcoded oligonucleotide may comprise a biotin moiety) or a nucleic acid hybridization linkage. Any one or more linker molecule may be a biopolymer (e.g. a nucleic acid molecule) or a synthetic polymer. Any one or more linker molecule may comprise one or more units of ethylene glycol and/or poly(ethylene) glycol (e.g. hexa-ethylene glycol or penta-ethylene glycol). Any one or more linker molecule may comprise one or more ethyl groups, such as one or more C3 (three-carbon) spacers, C6 spacers, C12 spacers, or C18 spacers.

The barcoded oligonucleotides may be synthesized by a chemical oligonucleotide synthesis process. The barcoded oligonucleotides synthesis process may include one or more step of an enzymatic production process, an enzymatic amplification process, or an enzymatic modification procedure, such as an in vitro transcription process, a reverse transcription process, a primer-extension process, or a polymerase chain reaction process.

These general properties of barcoded oligonucleotides are applicable to any of the multimeric barcoding reagents described herein.

### 3. GENERAL PROPERTIES OF CELL-BINDING MOIETIES

A cell-binding moiety may be comprised within a multimeric barcoding reagent, multimeric hybridization molecule, multimeric barcode molecule, hybridization molecule, barcode molecule, barcoded oligonucleotide and/or adapter oligonucleotide.

A cell-binding moiety may be attached (or linked) to a multimeric barcoding reagent, multimeric hybridization molecule, multimeric barcode molecule, hybridization molecule, barcode molecule, barcoded oligonucleotide and/or adapter oligonucleotide by a covalent linkage or by a non-covalent linkage (e.g. a protein-protein interaction or a streptavidin-biotin bond), nucleic acid hybridization, electrostatic interactions, via Van der Waals forces and/or hydrophobic interactions and/or via physisorption and/or chemisorption.

A cell-binding moiety may be attached (or linked) to a multimeric barcoding reagent, multimeric hybridization molecule, multimeric barcode molecule, hybridization molecule, barcode molecule, barcoded oligonucleotide and/or adapter oligonucleotide by a linker molecule. Optionally, said linker may be a flexible linker. The linker molecule may be a biopolymer (e.g. a nucleic acid molecule or peptide) or a synthetic polymer. The linker molecule may be a peptide-based polymers or a nucleic acid based polymer (e.g. PNA). The linker molecule may comprise one or more units of ethylene glycol and/or poly(ethylene) glycol (e.g. hexa-ethylene glycol or penta-ethylene glycol). The linker molecule may comprise one or more ethyl groups, such as a C3 (three-carbon) spacer, C6 spacer, C12 spacer, or C18 spacer. The linker molecule may comprise at least 2, at least 3, at least 4, at least 5, at least 10, or at least 20 or more sequential repeating units of any individual linker (such as a sequential linear series of at least 2, at least 5, or at least 10 C12 spacers or C18 spacers). The linker molecule may comprise a branched linker molecule, wherein 2 or more cell-binding moieties are linked to a multimeric barcoding reagent (or a multimeric hybridization molecule, multimeric barcode molecule, hybridization molecule, barcode molecule, barcoded oligonucleotide and/or adapter oligonucleotide) by a single linker molecule.

A cell-binding moiety may be attached (or linked) to a multimeric barcoding reagent, multimeric hybridization molecule, multimeric barcode molecule, hybridization molecule, barcode molecule, barcoded oligonucleotide and/or adapter oligonucleotide by one or more covalent linkage(s) (or bond(s)) (e.g. by a covalent bond such as a bond generated by any amino-modification attachment chemistry, and/or any carboxy-modification attachment chemistry, and/or any thiol-modification attachment chemistry, and/or any NHS-ester attachment chemistry, and/or any click-chemistry-related method, such as any copper (I)-catalysed azide-alkyne cycloaddition (CuAAC) reaction, a strain-promoted azide-alkyne cycloaddition (SPAAC) reaction, a strain-promoted alkyne-nitrone cycloaddition (SPANC) reaction, or an alkene and tetrazole photoclick reaction), one or more non-covalent linkages (or bond(s)) (e.g. a protein-protein interaction or a streptavidin-biotin linkage e.g. a support may comprise a streptavidin domain and a multimeric hybridization molecule (e.g. a multimeric barcode molecule) may comprise a biotin moiety), physisorption and/or chemisorption (e.g. thiol-Au interactions), electrostatic interactions (e.g. a polycationic polymer like poly-lysine interacting with a polyanionic polymer like DNA) or a nucleic acid hybridization linkage.

The cell-binding moiety (or moieties) may capable of initiating endocytosis on binding to a cell membrane.

The cell-binding moiety may comprise a hydrophobic moiety (e.g. cholesterol, palmitate and/or a phospholipid), a charged polymer (e.g. poly-lysine and/or or poly-arginine) and/or a target-specific macromolecule (e.g. an antibody and/or an aptamer).

The cell-binding moiety may comprise one or more moieties selected from: a peptide, a cell penetrating peptide, a pore forming peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a cationic lipid, a cationic polymer, poly(ethylene) glycol, spermine, a spermine derivatives or analogue, a poly-lysine, a poly-lysine derivative or analogue, a poly-arginine, a poly-arginine derivative or analogue, polyethyleneimine, diethylaminoethyl (DEAE)-dextran, cholesterol, a sterol moiety, a lipophilic antioxidant, a cationic molecule and/or polymer, a hydrophobic molecule, an amphiphilic molecule, any active site that may form a covalent bond with anything present on the cellular membrane (e.g. an NHS ester group capable of forming a peptide bond with amines present on cell surface proteins, thiols capable of forming disulfides with thiols/disulfides present on cell surface proteins, etc.) and a molecule with high affinity to a target added to the surface of the cell (e.g. an oligonucleotide complementary to another oligonucleotide conjugated to the surface of the cell, etc.).

The cell-binding moiety may interact with one or more specific molecule(s) on the cell surface (as in the case of e.g. an antibody, an antibody fragment and an aptamer). Alternatively or additionally, the cell-binding moiety may alter the overall charge and/or charge distribution of multimeric barcoding reagents (as in the case of e.g. a cationic polymer). Alternatively or additionally, the cell-binding moiety may alter the lipophilic/lipophobic and/or hydrophilic/hydrophobic character and/or balance of the multimeric barcoding reagents (as in the case of e.g. a lipid or cholesterol). Alternatively or additionally, the cell-binding moiety may form a covalent bond with anything present on the cellular membrane (e.g. an NHS ester group capable of forming a peptide bond with amines present on cell surface proteins, thiols capable of forming disulfides with thiols/disulfides present on cell surface proteins, etc.).

The cell-binding moiety may be a molecule that has a net positive charge in a solution comprising a cell and that enables binding of a multimeric barcoding reagent to the cell.

A multimeric barcoding reagent, multimeric hybridization molecule, multimeric barcode molecule, hybridization molecule, barcode molecule, barcoded oligonucleotide and/or adapter oligonucleotide may comprise at least 2, at least 3, at least 5, at least 10, at least 20, at least 50, at least 100, at least 500, or at least 1000 cell binding moieties.

Multimeric barcoding reagents may or may not contain one or more cell-binding moieties. The cell-binding moieties of a library of multimeric barcoding reagents, a multimeric barcoding reagent, a multimeric hybridization molecule or a multimeric barcode molecule may be identical or they may be different to all or some of the others. A cell-binding moiety may be located anywhere on a multimeric barcoding reagent; for example, a cell-binding moiety may be located on a support and/or on a multimeric hybridization molecule (e.g. on a multimeric barcode molecule) and/or on a barcoded oligonucleotide. Preferably, the cell-binding moiety is located on the exterior part of the multimeric barcoding reagent to facilitate interaction with a cell. The cell-binding moiety may be located at the 5′ or 3′ end of a multimeric hybridization molecule (e.g. a multimeric barcode molecule).

A cell-binding moiety may be a cationic molecule. Cationic molecules leverage the positive charge of the molecule to allow strong interactions to proteins, cell surfaces and DNA molecules. The cationic molecule may have a molecular weight of at least 1,000 daltons, at least 2,000 daltons, at least 5,000 daltons, at least 10,000 daltons, at least 20,000 daltons, at least 50,000 daltons, at least 100,000 daltons, at least 150,000 daltons, at least 300,000 daltons or at least 500,000 daltons. A distribution of cationic molecules having different molecular weights may be used.

The process of adhesion of the cationic molecules to support moieties is described herein as “priming”. Support moieties, such as, but not limited to, beads or alternative structures may be treated in a cationic molecule solution in order to prime them for cell-binding. Such a treatment step can be performed using a concentration of solution within a background buffer of, not limited to any of the following: PBS, PBS containing MgCl2 (for example at least 0.1 mM, at least 1 mM, at least 2 mM, at least 3 mM, at least 5 mM, at least 10 mM, at least 20 mM), Tris 7.5 buffer (for example at least 1 mM, at least 5 mM, at least 10 mM, at least 15 mM, at least 20 mM, at least 40 mM, at least 60 mM, at least 80 mM, at least 100 mM, at least 150 mM, at least 200 or more mM) or PBS containing polyethylene glycol 4000 (for example at least 1%, at least 2%, at least 5%, at least 10%, at least 15%, at least 20%, at least 30%). Concentrations of solution used during the priming protocol may be at least 0.0001%, at least 0.001%, at least 0.01%, at least 0.1%, at least 1%, at least 10%.

The priming protocol may be performed before or prior to the hybridization of barcoded oligonucleotides to one or more multimeric hybridization molecules. The priming may alternatively be performed during a target structure binding step (such as cell binding) within the same solution.

The priming protocol may be performed between 4° C. to 60° C. (for example at least 4° C., at least 10° C., at least 15° C., at least 20° C., at least 30° C., at least 40° C., at least 50° C., at least 60° C.). The priming protocol may be performed for, at least 1 minute, at least 10 minutes, at least 30 minutes, at least 1 hour, at least 2 hours, at least 4 hours, at least 8 hours or at least 16 hours. The priming protocol may utilise a rotational or vibrational mixing instrument to allow continuous in solution mixing during the priming reaction.

The priming protocol may be performed in the presence or absence or other cell binding moieties contained on the multimeric barcoding reagents. Multiple different cationic molecules may be used in the priming protocol simultaneously and may be used at different concentrations.

Following priming steps, the support(s) may be washed in a buffer of, not limited to any of the following: PBS, PBS containing at least 3 mM MgCl2 (for example at least 0.1 mM, at least 1 mM, at least 2 mM, at least 3 mM, at least 5 mM, at least 10 mM, at least 20 mM), Tris 7.5 buffer (for example at least 1 mM, at least 5 mM, at least 10 mM, at least 15 mM, at least 20 mM, at least 40 mM, at least 60 mM, at least 80 mM, at least 100 mM, at least 150 mM, at least 200 or more mM) or PBS containing polyethylene glycol 4000 (for example at least 1%, at least 2%, at least 5%, at least 10%, at least 15%, at least 20%, at least 30%). This wash may remove excess cationic molecules which have not adhered to the support moieties or multimeric barcoding reagents.

Cationic molecules may include, but are not limited to, Poly-lysine, Poly-arginine, Poly-amine containing molecules or cationic proteins. Where enantiomers exist, both L and D varieties may be utilised.

### 4. GENERAL PROPERTIES OF LIBRARIES OF MULTIMERIC BARCODING REAGENTS

The invention provides a library of multimeric barcoding reagents comprising first and second multimeric barcoding reagents as defined herein, wherein the barcode regions of the first multimeric barcoding reagent are different to the barcode regions of the second multimeric barcoding reagent.

The library of multimeric barcoding reagents may comprise at least 5, at least 10, at least 20, at least 25, at least 50, at least 75, at least 100, at least 250, at least 500, at least 10³, at least 10⁴, at least 10⁵, at least 10⁶, at least 10⁷, at least 10⁸ or at least 10⁹ multimeric barcoding reagents as defined herein. Preferably, the library comprises at least 10 multimeric barcoding reagents as defined herein. Preferably, the first and second barcode regions of each multimeric barcoding reagent are different to the barcode regions of at least 9 other multimeric barcoding reagents in the library.

The first and second barcode regions of each multimeric barcoding reagent may be different to the barcode regions of at least 4, at least 9, at least 19, at least 24, at least 49, at least 74, at least 99, at least 249, at least 499, at least 999 (i.e. 10³-1), at least 10⁴-1, at least 10⁵-1, at least 10⁶-1, at least 10⁷-1, at least 10⁸-1 or at least 10⁹-1 other multimeric barcoding reagents in the library. The first and second barcode regions of each multimeric barcoding reagent may be different to the barcode regions of all of the other multimeric barcoding reagents in the library. Preferably, the first and second barcode regions of each multimeric barcoding reagent are different to the barcode regions of at least 9 other multimeric barcoding reagents in the library.

The barcode regions of each multimeric barcoding reagent may be different to the barcode regions of at least 4, at least 9, at least 19, at least 24, at least 49, at least 74, at least 99, at least 249, at least 499, at least 999 (i.e. 10³-1), at least 10⁴-1, at least 10⁵-1, at least 10⁶-1, at least 10⁷-1, at least 10⁸-1 or at least 10⁹-1 other multimeric barcoding reagents in the library. The barcode regions of each multimeric barcoding reagent may be different to the barcode regions of all of the other multimeric barcoding reagents in the library. Preferably, the barcode regions of each multimeric barcoding reagent are different to the barcode regions of at least 9 other multimeric barcoding reagents in the library.

The invention provides a library of multimeric barcoding reagents comprising first and second multimeric barcoding reagents as defined herein, wherein the barcode regions of the barcoded oligonucleotides of the first multimeric barcoding reagent are different to the barcode regions of the barcoded oligonucleotides of the second multimeric barcoding reagent.

Different multimeric barcoding reagents within a library of multimeric barcoding reagents may comprise different numbers of barcoded oligonucleotides.

The library of multimeric barcoding reagents may comprise at least 5, at least 10, at least 20, at least 25, at least 50, at least 75, at least 100, at least 250, at least 500, at least 10³, at least 10⁴, at least 10⁵, at least 10⁶, at least 10⁷, at least 10⁸ or at least 10⁹ multimeric barcoding reagents as defined herein. Preferably, the library comprises at least 10 multimeric barcoding reagents as defined herein. Preferably, the barcode regions of the first and second barcoded oligonucleotides of each multimeric barcoding reagent are different to the barcode regions of the barcoded oligonucleotides of at least 9 other multimeric barcoding reagents in the library.

The barcode regions of the first and second barcoded oligonucleotides of each multimeric barcoding reagent may be different to the barcode regions of the barcoded oligonucleotides of at least 4, at least 9, at least 19, at least 24, at least 49, at least 74, at least 99, at least 249, at least 499, at least 999 (i.e. 10³-1), at least 10⁴-1, at least 10⁵-1, at least 10⁶-1, at least 10⁷-1, at least 10⁸-1 or at least 10⁹-1 other multimeric barcoding reagents in the library. The barcode regions of the first and second barcoded oligonucleotides of each multimeric barcoding reagent may be different to the barcode regions of the barcoded oligonucleotides of all of the other multimeric barcoding reagents in the library. Preferably, the barcode regions of the first and second barcoded oligonucleotides of each multimeric barcoding reagent are different to the barcode regions of the barcoded oligonucleotides of at least 9 other multimeric barcoding reagents in the library.

The barcode regions of the barcoded oligonucleotides of each multimeric barcoding reagent may be different to the barcode regions of the barcoded oligonucleotides of at least 4, at least 9, at least 19, at least 24, at least 49, at least 74, at least 99, at least 249, at least 499, at least 999 (i.e. 10³-1), at least 10⁴-1, at least 10⁵-1, at least 10⁶-1, at least 10⁷-1, at least 10⁸-1 or at least 10⁹-1 other multimeric barcoding reagents in the library. The barcode regions of the barcoded oligonucleotides of each multimeric barcoding reagent may be different to the barcode regions of the barcoded oligonucleotides of all of the other multimeric barcoding reagents in the library. Preferably, the barcode regions of the barcoded oligonucleotides of each multimeric barcoding reagent are different to the barcode regions of the barcoded oligonucleotides of at least 9 other multimeric barcoding reagents in the library.

These general properties of libraries of multimeric barcoding reagents are applicable to any of the multimeric barcoding reagents described herein.

### 5. MULTIMERIC BARCODING REAGENTS COMPRISING BARCODED OLIGONUCLEOTIDES ANNEALED TO A MULTIMERIC BARCODE MOLECULE

The invention provides a multimeric barcoding reagent for labelling a target nucleic acid, wherein the reagent comprises: first and second barcode molecules linked together (i.e. a multimeric barcode molecule), wherein each of the barcode molecules comprises a nucleic acid sequence comprising a barcode region; and first and second barcoded oligonucleotides, wherein the first barcoded oligonucleotide comprises, optionally in the 5′ to 3′ direction, a barcode region annealed to the barcode region of the first barcode molecule and a target region capable of annealing or ligating to a first sub-sequence of the target nucleic acid, and wherein the second barcoded oligonucleotide comprises, optionally in the 5′ to 3′ direction, a barcode region annealed to the barcode region of the second barcode molecule and a target region capable of annealing or ligating to a second sub-sequence of the target nucleic acid.

The invention provides a multimeric barcoding reagent for labelling a target nucleic acid, wherein the reagent comprises: first and second barcode molecules linked together (i.e. a multimeric barcode molecule), wherein each of the barcode molecules comprises a nucleic acid sequence comprising a barcode region; and first and second barcoded oligonucleotides, wherein the first barcoded oligonucleotide comprises a barcode region annealed to the barcode region of the first barcode molecule and a target region capable of ligating to a first sub-sequence of the target nucleic acid, and wherein the second barcoded oligonucleotide comprises a barcode region annealed to the barcode region of the second barcode molecule and a target region capable of ligating to a second sub-sequence of the target nucleic acid.

The invention provides a multimeric barcoding reagent for labelling a target nucleic acid, wherein the reagent comprises: first and second barcode molecules linked together (i.e. a multimeric barcode molecule), wherein each of the barcode molecules comprises a nucleic acid sequence comprising a barcode region; and first and second barcoded oligonucleotides, wherein the first barcoded oligonucleotide comprises in the 5′ to 3′ direction a barcode region annealed to the barcode region of the first barcode molecule and a target region capable of annealing to a first sub-sequence of the target nucleic acid, and wherein the second barcoded oligonucleotide comprises in the 5′ to 3′ direction a barcode region annealed to the barcode region of the second barcode molecule and a target region capable of annealing to a second sub-sequence of the target nucleic acid.

The invention provides a multimeric barcoding reagent for labelling a target nucleic acid, wherein the reagent comprises: first and second barcode molecules linked together (i.e. a multimeric barcode molecule), wherein each of the barcode molecules comprises a nucleic acid sequence comprising a barcode region; and first and second barcoded oligonucleotides, wherein the first barcoded oligonucleotide comprises a barcode region annealed to the barcode region of the first barcode molecule and capable of ligating to a first sub-sequence of the target nucleic acid, and wherein the second barcoded oligonucleotide comprises a barcode region annealed to the barcode region of the second barcode molecule and capable of ligating to a second sub-sequence of the target nucleic acid.

Each barcoded oligonucleotide may consist essentially of or consist of a barcode region.

Preferably, the barcode molecules comprise or consist of deoxyribonucleotides. One or more of the deoxyribonucleotides may be a modified deoxyribonucleotide (e.g. a deoxyribonucleotide modified with a biotin moiety or a deoxyuracil nucleotide). The barcode molecules may comprise one or more degenerate nucleotides or sequences. The barcode molecules may not comprise any degenerate nucleotides or sequences.

The barcode regions may uniquely identify each of the barcode molecules. Each barcode region may comprise a sequence that identifies the multimeric barcoding reagent. For example, this sequence may be a constant region shared by all barcode regions of a single multimeric barcoding reagent. Each barcode region may comprise at least 5, at least 10, at least 15, at least 20, at least 25, at least 50 or at least 100 nucleotides. Preferably, each barcode region comprises at least 5 nucleotides. Preferably each barcode region comprises deoxyribonucleotides, optionally all of the nucleotides in a barcode region are deoxyribonucleotides. One or more of the deoxyribonucleotides may be a modified deoxyribonucleotide (e.g. a deoxyribonucleotide modified with a biotin moiety or a deoxyuracil nucleotide). The barcode regions may comprise one or more degenerate nucleotides or sequences. The barcode regions may not comprise any degenerate nucleotides or sequences.

Preferably, the barcode region of the first barcoded oligonucleotide comprises a sequence that is complementary and annealed to the barcode region of the first barcode molecule and the barcode region of the second barcoded oligonucleotide comprises a sequence that is complementary and annealed to the barcode region of the second barcode molecule. The complementary sequence of each barcoded oligonucleotide may be at least 5, at least 10, at least 15, at least 20, at least 25, at least 50 or at least 100 contiguous nucleotides.

The target regions of the barcoded oligonucleotides (which are not annealed to the multimeric barcode molecule(s)) may be non-complementary to the multimeric barcode molecule(s).

The barcoded oligonucleotides may comprise a linker region between the barcode region and the target region. The linker region may comprise one or more contiguous nucleotides that are not annealed to the multimeric barcode molecule and are non-complementary to the subsequences of the target nucleic acid. The linker may comprise 1 to 100, 5 to 75, 10 to 50, 15 to 30 or 20 to 25 non-complementary nucleotides. Preferably, the linker comprises 15 to 30 non-complementary nucleotides. The use of such a linker region enhances the efficiency of the barcoding reactions performed using the multimeric barcoding reagents.

Barcode molecules may further comprise one or more nucleic acid sequences that are not complementary to barcode regions of barcoded oligonucleotides. For example, barcode molecules may comprise one or more adapter regions. A barcode molecule, may, for example, comprise an adapter region 5′ of a barcode region (a 5′ adapter region) and/or an adapter region 3′ of the barcode region (a 3′ adapter region). The adapter region(s) (and/or one or more portions of an adapter region) may be complementary to and anneal to oligonucleotides e.g. the adapter regions of barcoded oligonucleotides. Alternatively, the adapter region(s) (and/or one or more portions of an adapter region) of barcode molecule may not be complementary to sequences of barcoded oligonucleotides. The adapter region(s) may be used for manipulating, purifying, retrieving, amplifying, and/or detecting barcode molecules.

The multimeric barcoding reagent may be configured such that: each of the barcode molecules comprises a nucleic acid sequence comprising in the 5′ to 3′ direction an adapter region and a barcode region; the first barcoded oligonucleotide comprises, optionally in the 5′ to 3′ direction, a barcode region annealed to the barcode region of the first barcode molecule, an adapter region annealed to the adapter region of the first barcode molecule and a target region capable of annealing to a first sub-sequence of the target nucleic acid; and the second barcoded oligonucleotide comprises, optionally in the 5′ to 3′ direction, a barcode region annealed to the barcode region of the second barcode molecule, an adapter region annealed to the adapter region of the second barcode molecule and a target region capable of annealing to a second sub-sequence of the target nucleic acid.

The adapter region of each barcode molecule may comprise a constant region. Optionally, all adapter regions of a multimeric barcoding reagent are substantially identical. The adapter region may comprise at least 1, at least 2, at least 3, at least 4, at least 5, at least 6, at least 8, at least 10, at least 15, at least 20, at least 25, at least 50, at least 100, or at least 250 nucleotides. Preferably, the adapter region comprises at least 4 nucleotides. Preferably each adapter region comprises deoxyribonucleotides, optionally all of the nucleotides in an adapter region are deoxyribonucleotides. One or more of the deoxyribonucleotides may be a modified deoxyribonucleotide (e.g. a deoxyribonucleotide modified with a biotin moiety or a deoxyuracil nucleotide). Each adapter region may comprise one or more universal bases (e.g. inosine), one or modified nucleotides and/or one or more nucleotide analogues.

The barcoded oligonucleotides may comprise a linker region between the adapter region and the target region. The linker region may comprise one or more contiguous nucleotides that are not annealed to the multimeric barcode molecule and are non-complementary to the subsequences of the target nucleic acid. The linker may comprise 1 to 100, 5 to 75, 10 to 50, 15 to 30 or 20 to 25 non-complementary nucleotides. Preferably, the linker comprises 15 to 30 non-complementary nucleotides. The use of such a linker region enhances the efficiency of the barcoding reactions performed using the multimeric barcoding reagents.

The barcode molecules of a multimeric barcode molecule may be linked on a nucleic acid molecule. Such a nucleic acid molecule may provide the backbone to which single-stranded barcoded oligonucleotides may be annealed. Alternatively, the barcode molecules of a multimeric barcode molecule may be linked together by any of the other means described herein.

The multimeric barcoding reagent may comprise: at least 2, at least 3, at least 4, at least 5, at least 10, at least 20, at least 25, at least 50, at least 75, at least 100, at least 200, at least 500, at least 1000, at least 5000, or at least 10,000 barcode molecules linked together, wherein each barcode molecule is as defined herein; and a barcoded oligonucleotide annealed to each barcode molecule, wherein each barcoded oligonucleotide is as defined herein. Preferably, the multimeric barcoding reagent comprises at least 5 barcode molecules linked together, wherein each barcode molecule is as defined herein; and a barcoded oligonucleotide annealed to each barcode molecule, wherein each barcoded oligonucleotide is as defined herein.

The multimeric barcoding reagent may comprise: at least 2, at least 3, at least 4, at least 5, at least 10, at least 20, at least 25, at least 50, at least 75, at least 100, at least 200, at least 500, at least 1000, at least 5000, at least 10⁴, at least 10⁵, or at least 10⁶ unique or different barcode molecules linked together, wherein each barcode molecule is as defined herein; and a barcoded oligonucleotide annealed to each barcode molecule, wherein each barcoded oligonucleotide is as defined herein. Preferably, the multimeric barcoding reagent comprises at least 5 unique or different barcode molecules linked together, wherein each barcode molecule is as defined herein; and a barcoded oligonucleotide annealed to each barcode molecule, wherein each barcoded oligonucleotide is as defined herein.

The multimeric barcoding reagent may comprise: at least 5, at least 10, at least 20, at least 25, at least 50, at least 75, at least 100, at least 200, at least 500, at least 1000, at least 5000, or at least 10,000 barcode regions, wherein each barcode region is as defined herein; and a barcoded oligonucleotide annealed to each barcode region, wherein each barcoded oligonucleotide is as defined herein. Preferably, the multimeric barcoding reagent comprises at least 5 barcode regions, wherein each barcode region is as defined herein; and a barcoded oligonucleotide annealed to each barcode region, wherein each barcoded oligonucleotide is as defined herein.

The multimeric barcoding reagent may comprise: at least 2, at least 3, at least 4, at least 5, at least 10, at least 20, at least 25, at least 50, at least 75, at least 100, at least 200, at least 500, at least 1000, at least 5000, at least 10⁴, at least 10⁵, or at least 10⁶ unique or different barcode regions, wherein each barcode region is as defined herein; and a barcoded oligonucleotide annealed to each barcode region, wherein each barcoded oligonucleotide is as defined herein. Preferably, the multimeric barcoding reagent comprises at least 5 unique or different barcode regions, wherein each barcode region is as defined herein; and a barcoded oligonucleotide annealed to each barcode region, wherein each barcoded oligonucleotide is as defined herein.

FIG. 1 shows a multimeric barcoding reagent, including first (D1, E1, and F1) and second (D2, E2, and F2) barcode molecules, which each include a nucleic acid sequence comprising a barcode region (E1 and E2). These first and second barcode molecules are linked together, for example by a connecting nucleic acid sequence(S). The multimeric barcoding reagent also comprises first (A1, B1, C1, and G1) and second (A2, B2, C2, and G2) barcoded oligonucleotides. These barcoded oligonucleotides each comprise a barcode region (B1 and B2) and a target region (G1 and G2).

The barcode regions within the barcoded oligonucleotides may each contain a unique sequence which is not present in other barcoded oligonucleotides, and may thus serve to uniquely identify each such barcode molecule. The target regions may be used to anneal the barcoded oligonucleotides to sub-sequences of target nucleic acids, and then may be used as primers for a primer-extension reaction or an amplification reaction e.g. a polymerase chain reaction.

Each barcode molecule may optionally also include a 5′ adapter region (F1 and F2). The barcoded oligonucleotides may then also include a 3′ adapter region (C1 and C2) that is complementary to the 5′ adapter region of the barcode molecules.

Each barcode molecule may optionally also include a 3′ region (D1 and D2), which may be comprised of identical sequences within each barcode molecule. The barcoded oligonucleotides may then also include a 5′ region (A1 and A2) which is complementary to the 3′ region of the barcode molecules. These 3′ regions may be useful for manipulation or amplification of nucleic acid sequences, for example sequences that are generated by labeling a nucleic acid target with a barcoded oligonucleotide. The 3′ region may comprise at least 4, at least 5, at least 6, at least 8, at least 10, at least 15, at least 20, at least 25, at least 50, at least 100, or at least 250 nucleotides. Preferably, the 3′ region comprises at least 4 nucleotides. Preferably each 3′ region comprises deoxyribonucleotides, optionally all of the nucleotides in an 3′ region are deoxyribonucleotides. One or more of the deoxyribonucleotides may be a modified deoxyribonucleotide (e.g. a deoxyribonucleotide modified with a biotin moiety or a deoxyuracil nucleotide). Each 3′ region may comprise one or more universal bases (e.g. inosine), one or modified nucleotides and/or one or more nucleotide analogues.

The invention provides a library of multimeric barcoding reagents comprising at least 10 multimeric barcoding reagents for labelling a target nucleic acid for sequencing, wherein each multimeric barcoding reagent comprises: first and second barcode molecules comprised within a (single) nucleic acid molecule, wherein each of the barcode molecules comprises a nucleic acid sequence comprising a barcode region; and first and second barcoded oligonucleotides, wherein the first barcoded oligonucleotide comprises, optionally in the 5′ to 3′ direction, a barcode region complementary and annealed to the barcode region of the first barcode molecule and a target region capable of annealing or ligating to a first sub-sequence of the target nucleic acid, and wherein the second barcoded oligonucleotide comprises, optionally in the 5′ to 3′ direction, a barcode region complementary and annealed to the barcode region of the second barcode molecule and a target region capable of annealing or ligating to a second sub-sequence of the target nucleic acid. Preferably, the barcode regions of the first and second barcoded oligonucleotides of each multimeric barcoding reagent are different to the barcode regions of the barcoded oligonucleotides of at least 9 other multimeric barcoding reagents in the library.

### 6. MULTIMERIC BARCODING REAGENTS COMPRISING BARCODED OLIGONUCLEOTIDES ANNEALED TO A MULTIMERIC HYBRIDIZATION MOLECULE

The invention provides a multimeric barcoding reagent for labelling a target nucleic acid, wherein the reagent comprises: first and second hybridization molecules linked together (i.e. a multimeric hybridization molecule), wherein each of the hybridization molecules comprises a nucleic acid sequence comprising a hybridization region; and first and second barcoded oligonucleotides, wherein the first barcoded oligonucleotide comprises, optionally in the 5′ to 3′ direction, an adapter region annealed to the hybridization region of the first hybridization molecule, a barcode region, and a target region capable of annealing or ligating to a first sub-sequence of the target nucleic acid, and wherein the second barcoded oligonucleotide comprises, optionally in the 5′ to 3′ direction, an adapter region annealed to the hybridization region of the second hybridization molecule, a barcode region, and a target region capable of annealing or ligating to a second sub-sequence of the target nucleic acid.

Optionally, the first and second barcoded oligonucleotides each comprise an adapter region and a target region in a single contiguous sequence that is complementary and annealed to a hybridization region of a hybridization molecule, and also capable of annealing or ligating to a sub-sequence of a target nucleic acid.

The invention provides a multimeric barcoding reagent for labelling a target nucleic acid, wherein the reagent comprises: first and second hybridization molecules linked together (i.e. a multimeric hybridization molecule), wherein each of the hybridization molecules comprises a nucleic acid sequence comprising a hybridization region; and first and second barcoded oligonucleotides, wherein the first barcoded oligonucleotide comprises, optionally in the 5′ to 3′ direction, a barcode region, an adapter region annealed to the hybridization region of the first hybridization molecule and a target region capable of annealing or ligating to a first sub-sequence of the target nucleic acid, and wherein the second barcoded oligonucleotide comprises, optionally in the 5′ to 3′ direction, a barcode region, an adapter region annealed to the hybridization region of the second hybridization molecule and a target region capable of annealing or ligating to a second sub-sequence of the target nucleic acid.

Optionally, the first and second barcoded oligonucleotides each comprise an adapter region and a target region in a single contiguous sequence that is complementary and annealed to a hybridization region of a hybridization molecule, and also capable of annealing or ligating to a sub-sequence of a target nucleic acid.

The invention provides a multimeric barcoding reagent for labelling a target nucleic acid, wherein the reagent comprises: first and second hybridization molecules linked together (i.e. a multimeric hybridization molecule), wherein each of the hybridization molecules comprises a nucleic acid sequence comprising a hybridization region; and first and second barcoded oligonucleotides, wherein the first barcoded oligonucleotide comprises (in the 5′-3′ or 3′-5′ direction) an adapter region annealed to the hybridization region of the first hybridization molecule, a barcode region and a target region capable of ligating to a first sub-sequence of the target nucleic acid, and wherein the second barcoded oligonucleotide comprises (in the 5′-3′ or 3′-5′ direction) an adapter region annealed to the hybridization region of the second hybridization molecule, a barcode region and a target region capable of ligating to a second sub-sequence of the target nucleic acid.

Optionally, the first and second barcoded oligonucleotides each comprise an adapter region and a target region in a single contiguous sequence that is complementary and annealed to a hybridization region of a hybridization molecule, and also capable of ligating to a sub-sequence of a target nucleic acid.

The invention provides a multimeric barcoding reagent for labelling a target nucleic acid, wherein the reagent comprises: first and second hybridization molecules linked together (i.e. a multimeric hybridization molecule), wherein each of the hybridization molecules comprises a nucleic acid sequence comprising a hybridization region; and first and second barcoded oligonucleotides, wherein the first barcoded oligonucleotide comprises (in the 5′-3′ or 3′-5′ direction) a barcode region, an adapter region annealed to the hybridization region of the first hybridization molecule and a target region capable of ligating to a first sub-sequence of the target nucleic acid, and wherein the second barcoded oligonucleotide comprises (in the 5′-3′ or 3′-5′ direction) a barcode region, an adapter region annealed to the hybridization region of the second hybridization molecule and a target region capable of ligating to a second sub-sequence of the target nucleic acid.

Optionally, the first and second barcoded oligonucleotides each comprise an adapter region and a target region in a single contiguous sequence that is complementary and annealed to a hybridization region of a hybridization molecule, and also capable of ligating to a sub-sequence of a target nucleic acid.

The invention provides a multimeric barcoding reagent for labelling a target nucleic acid, wherein the reagent comprises: first and second hybridization molecules linked together (i.e. a multimeric hybridization molecule), wherein each of the hybridization molecules comprises a nucleic acid sequence comprising a barcode region; and first and second barcoded oligonucleotides, wherein the first barcoded oligonucleotide comprises in the 5′ to 3′ direction an adapter region annealed to the hybridization region of the first hybridization molecule, a barcode region and a target region capable of annealing to a first sub-sequence of the target nucleic acid, and wherein the second barcoded oligonucleotide comprises in the 5′ to 3′ direction an adapter region annealed to the hybridization region of the second hybridization molecule, a barcode region and a target region capable of annealing to a second sub-sequence of the target nucleic acid.

The invention provides a multimeric barcoding reagent for labelling a target nucleic acid, wherein the reagent comprises: first and second hybridization molecules linked together (i.e. a multimeric hybridization molecule), wherein each of the hybridization molecules comprises a nucleic acid sequence comprising a barcode region; and first and second barcoded oligonucleotides, wherein the first barcoded oligonucleotide comprises in the 5′ to 3′ direction a barcode region, an adapter region annealed to the hybridization region of the first hybridization molecule and a target region capable of annealing to a first sub-sequence of the target nucleic acid, and wherein the second barcoded oligonucleotide comprises in the 5′ to 3′ direction a barcode region, an adapter region annealed to the hybridization region of the second hybridization molecule and a target region capable of annealing to a second sub-sequence of the target nucleic acid.

Optionally, the first and second barcoded oligonucleotides each comprise an adapter region and a target region in a single contiguous sequence that is complementary and annealed to a hybridization region of a hybridization molecule, and also capable of annealing to a sub-sequence of a target nucleic acid.

Preferably, the adapter region of the first barcoded oligonucleotide comprises a sequence that is complementary and annealed to the hybridization region of the first hybridization molecule and the adapter region of the second barcoded oligonucleotide comprises a sequence that is complementary and annealed to the hybridization region of the second hybridization molecule. The complementary sequence of each barcoded oligonucleotide may be at least 5, at least 10, at least 15, at least 20, at least 25, at least 50 or at least 100 contiguous nucleotides.

The hybridization region of each hybridization molecule may comprise a constant region. Preferably, all hybridization regions of a multimeric barcoding reagent are substantially identical. Optionally, all hybridization regions of a library of multimeric barcoding reagents are substantially identical. The hybridization region may comprise at least 1, at least 2, at least 3, at least 4, at least 5, at least 6, at least 8, at least 10, at least 15, at least 20, at least 25, at least 50, at least 100, or at least 250 nucleotides. Preferably, the hybridization region comprises at least 4 nucleotides. Preferably each hybridization region comprises deoxyribonucleotides, optionally all of the nucleotides in a hybridization region are deoxyribonucleotides. One or more of the deoxyribonucleotides may be a modified deoxyribonucleotide (e.g. a deoxyribonucleotide modified with a biotin moiety or a deoxyuracil nucleotide). Each hybridization region may comprise one or more universal bases (e.g. inosine), one or modified nucleotides and/or one or more nucleotide analogues.

The target regions of the barcoded oligonucleotides may not be annealed to the multimeric hybridization molecule(s). The target regions of the barcoded oligonucleotides may be non-complementary to the multimeric hybridization molecule(s).

The barcoded oligonucleotides may comprise a linker region between the adapter region and the target region. The linker region may comprise one or more contiguous nucleotides that are not annealed to the multimeric hybridization molecule and are non-complementary to the subsequences of the target nucleic acid. The linker may comprise 1 to 100, 5 to 75, 10 to 50, 15 to 30 or 20 to 25 non-complementary nucleotides. Preferably, the linker comprises 15 to 30 non-complementary nucleotides. The use of such a linker region enhances the efficiency of the barcoding reactions performed using the multimeric barcoding reagents.

Hybridization molecules may further comprise one or more nucleic acid sequences that are not complementary to barcoded oligonucleotides. For example, hybridization molecules may comprise one or more adapter regions. A hybridization molecule, may, for example, comprise an adapter region 5′ of a hybridization region (a 5′ adapter region) and/or an adapter region 3′ of the hybridization region (a 3′ adapter region). The adapter region(s) may be used for manipulating, purifying, retrieving, amplifying, and/or detecting hybridization molecules.

The adapter region of each hybridization molecule may comprise a constant region. Optionally, all adapter regions of a multimeric hybridization reagent are substantially identical. The adapter region may comprise at least 1, at least 2, at least 3, at least 4, at least 5, at least 6, at least 8, at least 10, at least 15, at least 20, at least 25, at least 50, at least 100, or at least 250 nucleotides. Preferably, the adapter region comprises at least 4 nucleotides. Preferably each adapter region comprises deoxyribonucleotides, optionally all of the nucleotides in an adapter region are deoxyribonucleotides. One or more of the deoxyribonucleotides may be a modified deoxyribonucleotide (e.g. a deoxyribonucleotide modified with a biotin moiety or a deoxyuracil nucleotide). Each adapter region may comprise one or more universal bases (e.g. inosine), one or modified nucleotides and/or one or more nucleotide analogues.

The barcoded oligonucleotides may comprise a linker region between the adapter region and the target region. The linker region may comprise one or more contiguous nucleotides that are not annealed to the multimeric hybridization molecule and are non-complementary to the subsequences of the target nucleic acid. The linker may comprise 1 to 100, 5 to 75, 10 to 50, 15 to 30 or 20 to 25 non-complementary nucleotides. Preferably, the linker comprises 15 to 30 non-complementary nucleotides. The use of such a linker region enhances the efficiency of the barcoding reactions performed using the multimeric barcoding reagents.

The invention provides a library of multimeric barcoding reagents comprising at least 10 multimeric barcoding reagents for labelling a target nucleic acid for sequencing, wherein each multimeric barcoding reagent comprises: first and second hybridization molecules comprised within a (single) nucleic acid molecule, wherein each of the hybridization molecules comprises a nucleic acid sequence comprising a hybridization region; and first and second barcoded oligonucleotides, wherein the first barcoded oligonucleotide comprises, optionally in the 5′ to 3′ direction, an adapter region complementary and annealed to the hybridization region of the first hybridization molecule, a barcode region and a target region capable of annealing or ligating to a first sub-sequence of the target nucleic acid, and wherein the second barcoded oligonucleotide comprises, optionally in the 5′ to 3′ direction, an adapter region complementary and annealed to the hybridization region of the second hybridization molecule, a barcode region and a target region capable of annealing or ligating to a second sub-sequence of the target nucleic acid.

Preferably, the barcode regions of the first and second barcoded oligonucleotides of each multimeric barcoding reagent are different to the barcode regions of the barcoded oligonucleotides of at least 9 other multimeric barcoding reagents in the library.

The invention provides a library of multimeric barcoding reagents comprising at least 10 multimeric barcoding reagents for labelling a target nucleic acid for sequencing, wherein each multimeric barcoding reagent comprises: first and second hybridization molecules comprised within a (single) nucleic acid molecule, wherein each of the hybridization molecules comprises a nucleic acid sequence comprising a hybridization region; and first and second barcoded oligonucleotides, wherein the first barcoded oligonucleotide comprises, optionally in the 5′ to 3′ direction, a barcode region, an adapter region complementary and annealed to the hybridization region of the first hybridization molecule and a target region capable of annealing or ligating to a first sub-sequence of the target nucleic acid, and wherein the second barcoded oligonucleotide comprises, optionally in the 5′ to 3′ direction, a barcode region, an adapter region complementary and annealed to the hybridization region of the second hybridization molecule and a target region capable of annealing or ligating to a second sub-sequence of the target nucleic acid. Preferably, the barcode regions of the first and second barcoded oligonucleotides of each multimeric barcoding reagent are different to the barcode regions of the barcoded oligonucleotides of at least 9 other multimeric barcoding reagents in the library.

### 7. MULTIMERIC BARCODING REAGENTS COMPRISING BARCODED OLIGONUCLEOTIDES LINKED BY A MACROMOLECULE

The invention provides a multimeric barcoding reagent for labelling a target nucleic acid, wherein the reagent comprises first and second barcoded oligonucleotides linked together by a macromolecule, and wherein the barcoded oligonucleotides each comprise a barcode region.

The first barcoded oligonucleotide may further comprise a target region capable of annealing or ligating to a first sub-sequence of the target nucleic acid, and the second barcoded oligonucleotide may further comprise a target region capable of annealing or ligating to a second sub-sequence of the target nucleic acid.

The first barcoded oligonucleotide may comprise in the 5′-3′ direction a barcode region and a target region capable of annealing to a first sub-sequence of the target nucleic acid, and the second barcoded oligonucleotide may comprise in the 5′-3′ direction a barcode region and a target region capable of annealing to a second sub-sequence of the target nucleic acid.

The barcoded oligonucleotides may further comprise any of the features described herein.

The barcoded oligonucleotides may be linked by a macromolecule by being bound to the macromolecule and/or by being annealed to the macromolecule.

The barcoded oligonucleotides may be linked to the macromolecule directly or indirectly (e.g. via a linker molecule). The barcoded oligonucleotides may be linked by being bound to the macromolecule and/or by being bound or annealed to linker molecules that are bound to the macromolecule. The barcoded oligonucleotides may be bound to the macromolecule (or to the linker molecules) by covalent linkage, non-covalent linkage (e.g. a protein-protein interaction or a streptavidin-biotin bond) or nucleic acid hybridization. The linker molecule may be a biopolymer (e.g. a nucleic acid molecule) or a synthetic polymer. The linker molecule may comprise one or more units of ethylene glycol and/or poly(ethylene) glycol (e.g. hexa-ethylene glycol or penta-ethylene glycol). The linker molecule may comprise one or more ethyl groups, such as a C3 (three-carbon) spacer, C6 spacer, C12 spacer, or C18 spacer.

The macromolecule may be a synthetic polymer (e.g. a dendrimer) or a biopolymer such as a nucleic acid (e.g. a single-stranded nucleic acid such as single-stranded DNA), a peptide, a polypeptide or a protein (e.g. a multimeric protein).

The dendrimer may comprise at least 2, at least 3, at least 5, or at least 10 generations.

The macromolecule may be a nucleic acid comprising two or more nucleotides each capable of binding to a barcoded oligonucleotide. Additionally or alternatively, the nucleic acid may comprise two or more regions each capable of hybridizing to a barcoded oligonucleotide.

The nucleic acid may comprise a first modified nucleotide and a second modified nucleotide, wherein each modified nucleotide comprises a binding moiety (e.g. a biotin moiety, or an alkyne moiety which may be used for a click-chemical reaction) capable of binding to a barcoded oligonucleotide. Optionally, the first and second modified nucleotides may be separated by an intervening nucleic acid sequence of at least one, at least two, at least 5 or at least 10 nucleotides.

The nucleic acid may comprise a first hybridization region and a second hybridization region, wherein each hybridization region comprises a sequence complementary to and capable of hybridizing to a sequence of at least one nucleotide within a barcoded oligonucleotide. The complementary sequence may be at least 5, at least 10, at least 15, at least 20, at least 25 or at least 50 contiguous nucleotides. Optionally, the first and second hybridization regions may be separated by an intervening nucleic acid sequence of at least one, at least two, at least 5 or at least 10 nucleotides.

The macromolecule may be a protein such as a multimeric protein e.g. a homomeric protein or a heteromeric protein. For example, the protein may comprise streptavidin e.g. tetrameric streptavidin.

Libraries of multimeric barcoding reagents comprising barcoded oligonucleotides linked by a macromolecule are also provided. Such libraries may be based on the general properties of libraries of multimeric barcoding reagents described herein. In the libraries, each multimeric barcoding reagent may comprise a different macromolecule.

### 8. MULTIMERIC BARCODING REAGENTS COMPRISING BARCODED OLIGONUCLEOTIDES LINKED BY A SOLID SUPPORT OR A SEMI-SOLID SUPPORT

The invention provides a multimeric barcoding reagent for labelling a target nucleic acid, wherein the reagent comprises first and second barcoded oligonucleotides linked together by a solid support or a semi-solid support, and wherein the barcoded oligonucleotides each comprise a barcode region.

The first barcoded oligonucleotide may further comprise a target region capable of annealing or ligating to a first sub-sequence of the target nucleic acid, and the second barcoded oligonucleotide may further comprise a target region capable of annealing or ligating to a second sub-sequence of the target nucleic acid.

The first barcoded oligonucleotide may comprise in the 5′-3′ direction a barcode region and a target region capable of annealing to a first sub-sequence of the target nucleic acid, and the second barcoded oligonucleotide may comprise in the 5′-3′ direction a barcode region and a target region capable of annealing to a second sub-sequence of the target nucleic acid.

The barcoded oligonucleotides may further comprise any of the features described herein.

The barcoded oligonucleotides may be linked by a solid support or a semi-solid support. The barcoded oligonucleotides may be linked to the support directly or indirectly (e.g. via a linker molecule). The barcoded oligonucleotides may be linked by being bound to the support and/or by being bound or annealed to linker molecules that are bound to the support. The barcoded oligonucleotides may be bound to the support (or to the linker molecules) by covalent linkage, non-covalent linkage (e.g. a protein-protein interaction or a streptavidin-biotin bond) or nucleic acid hybridization. The linker molecule may be a biopolymer (e.g. a nucleic acid molecule) or a synthetic polymer. The linker molecule may comprise one or more units of ethylene glycol and/or poly(ethylene) glycol (e.g. hexa-ethylene glycol or penta-ethylene glycol). The linker molecule may comprise one or more ethyl groups, such as a C3 (three-carbon) spacer, C6 spacer, C12 spacer, or C18 spacer.

The support may comprise a planar surface. The support may be a slide e.g. a glass slide. The slide may be a flow cell for sequencing. If the support is a slide, the first and second barcoded oligonucleotides may be immobilized in a discrete region on the slide. Optionally, the barcoded oligonucleotides of each multimeric barcoding reagent in a library are immobilized in a different discrete region on the slide to the barcoded oligonucleotides of the other multimeric barcoding reagents in the library. The support may be a plate comprising wells, optionally wherein the first and second barcoded oligonucleotides are immobilized in the same well. Optionally, the barcoded oligonucleotides of each multimeric barcoding reagent in library are immobilized in a different well of the plate to the barcoded oligonucleotides of the other multimeric barcoding reagents in the library.

Preferably, the support is a bead (e.g. a gel bead). The bead may be an agarose bead, a silica bead, a styrofoam bead, a gel bead (such as those available from 10× Genomics®), an antibody conjugated bead, an oligo-dT conjugated bead, a streptavidin bead or a magnetic bead (e.g. a superparamagnetic bead). The bead may be of any size and/or molecular structure. For example, the bead may be 10 nanometres to 100 microns in diameter, 100 nanometres to 10 microns in diameter, or 1 micron to 5 microns in diameter. Optionally, the bead is approximately 10 nanometres in diameter, approximately 100 nanometres in diameter, approximately 1 micron in diameter, approximately 10 microns in diameter or approximately 100 microns in diameter. The bead may be solid, or alternatively the bead may be hollow or partially hollow or porous. Beads of certain sizes may be most preferable for certain barcoding methods. For example, beads less than 5.0 microns, or less than 1.0 micron, may be most useful for barcoding nucleic acid targets within individual cells. Preferably, the barcoded oligonucleotides of each multimeric barcoding reagent in a library are linked together on a different bead to the barcoded oligonucleotides of the other multimeric barcoding reagents in the library.

The support may be functionalised to enable attachment of two or more barcoded oligonucleotides. This functionalisation may be enabled through the addition of chemical moieties (e.g. carboxylated groups, alkynes, azides, acrylate groups, amino groups, sulphate groups, or succinimide groups), and/or protein-based moieties (e.g. streptavidin, avidin, or protein G) to the support. The barcoded oligonucleotides may be attached to the moieties directly or indirectly (e.g. via a linker molecule).

Functionalised supports (e.g. beads) may be brought into contact with a solution of barcoded oligonucleotides under conditions which promote the attachment of two or more barcoded oligonucleotides to each bead in the solution (generating multimeric barcoding reagents).

Libraries of multimeric barcoding reagents comprising barcoded oligonucleotides linked by a support are also provided. Such libraries may be based on the general properties of libraries of multimeric barcoding reagents described herein. In the libraries, each multimeric barcoding reagent may comprise a different support (e.g. a differently labelled bead). In a library of multimeric barcoding reagents, the barcoded oligonucleotides of each multimeric barcoding reagent in a library may be linked together on a different support to the barcoded oligonucleotides of the other multimeric barcoding reagents in the library.

### 9. MULTIMERIC BARCODING REAGENTS COMPRISING BARCODED OLIGONUCLEOTIDES LINKED TOGETHER BY BEING COMPRISED WITHIN A LIPID CARRIER

The invention provides a multimeric barcoding reagent for labelling a target nucleic acid, wherein the reagent comprises first and second barcoded oligonucleotides and a lipid carrier, wherein the first and second barcoded oligonucleotides are linked together by being comprised within the lipid carrier, and wherein the barcoded oligonucleotides each comprise a barcode region.

The first barcoded oligonucleotide may further comprise a target region capable of annealing or ligating to a first sub-sequence of the target nucleic acid, and the second barcoded oligonucleotide may further comprise a target region capable of annealing or ligating to a second sub-sequence of the target nucleic acid.

The first barcoded oligonucleotide may comprise in the 5′-3′ direction a barcode region and a target region capable of annealing to a first sub-sequence of the target nucleic acid, and the second barcoded oligonucleotide may comprise in the 5′-3′ direction a barcode region and a target region capable of annealing to a second sub-sequence of the target nucleic acid.

The barcoded oligonucleotides may further comprise any of the features described herein.

The invention provides a library of multimeric barcoding reagents comprising first and second multimeric barcoding reagents as defined herein, wherein the barcoded oligonucleotides of the first multimeric barcoding reagent are comprised within a first lipid carrier, and wherein the barcoded oligonucleotides of the second multimeric barcoding reagent are comprised with a second lipid carrier, and wherein the barcode regions of the barcoded oligonucleotides of the first multimeric barcoding reagent are different to the barcode regions of the barcoded oligonucleotides of the second multimeric barcoding reagent.

The library of multimeric barcoding reagents may comprise at least 5, at least 10, at least 20, at least 25, at least 50, at least 75, at least 100, at least 250, at least 500, at least 10³, at least 10⁴, at least 10⁵, at least 10⁶, at least 10⁷, at least 10⁸ or at least 10⁹ multimeric barcoding reagents as defined herein. Preferably, the library comprises at least 10 multimeric barcoding reagents as defined herein. Preferably, the barcode regions of the first and second barcoded oligonucleotides of each multimeric barcoding reagent are different to the barcode regions of the barcoded oligonucleotides of at least 9 other multimeric barcoding reagents in the library.

The barcoded oligonucleodides of each multimeric barcoding reagent are comprised within a different lipid carrier.

The lipid carrier may be a liposome or a micelle. The lipid carrier may be a phospholipid carrier. The lipid carrier may comprise one or more amphiphilic molecules. The lipid carrier may comprise one or more phospholipids. The phospholipid may be phosphatidylcholine. The lipid carrier may comprise one or more of the following constituents: phophatidylethanolamine, phosphatidylserine, cholesterol, cardiolipin, dicetylphosphate, stearylamine, phosphatidylglycerol, dipalmitoylphosphatidylcholine, distearylphosphatidylcholine, and/or any related and/or derivative molecules thereof. Optionally, the lipid carrier may comprise any combination of two or more constituents described above, with or without further constituents.

The lipid carrier (e.g. a liposome or a micelle) may be unilamellar or multilamellar. A library of multimeric barcoding reagents may comprise both unilamellar and multilamellar lipid carriers. The lipid carrier may comprise a copolymer e.g. a block copolymer.

The lipid carrier may comprise at least 2, at least 3, at least 5, at least 10, at least 50, at least 100, at least 500, at least 1000, at least 10,000, or at least 100,000 barcoded oligonucleotides, or any greater number of barcoded oligonucleotides.

Any lipid carrier (e.g. liposome or micelle, and/or liposomal or micellar reagent) may on average be complexed with 1, or less than 1, or greater than 1 multimeric barcoding reagent(s) to form a library of such multimeric barcoding reagent(s).

The invention provides a library of multimeric barcoding reagents comprising at least 10 multimeric barcoding reagents as defined herein, wherein each multimeric barcoding reagent comprises first and second barcoded oligonucleotides comprised within a different lipid carrier, and wherein the barcode regions of the first and second barcoded oligonucleotides of each multimeric barcoding reagent are different to the barcode regions of the barcoded oligonucleotides of at least 9 other multimeric barcoding reagents in the library.

A method for preparing multimeric barcoding reagents comprises loading barcoded oligonucleotides and/or multimeric barcoding reagent(s) into lipid carriers (e.g. liposomes or micelles). The method may comprise a step of passive, active, and/or remote loading. Pre-formed lipid carriers (e.g. liposomes and/or micelles) may be loaded by contacting them with a solution of barcoded oligonucleotides and/or multimeric barcoding reagent(s). Lipid carriers (e.g. liposomes and/or micelles) may be loaded by contacting them with a solution of barcoded oligonucleotides and/or multimeric barcoding reagent(s) prior to and/or during the formation or synthesis of the lipid carriers. The method may comprise passive encapsulation and/or trapping of barcoded oligonucleotides and/or multimeric barcoding reagent(s) in lipid carriers.

Lipid carriers (e.g. liposomes and/or micelles) may be prepared by a method based on sonication, a French press-based method, a reverse phase method, a solvent evaporation method, an extrusion-based method, a mechanical mixing-based method, a freeze/thaw-based method, a dehydrate/rehydrate-based method, and/or any combination hereof.

Lipid carriers (e.g. liposomes and/or micelles) may be stabilized and/or stored prior to use using known methods.

Any of the multimeric barcoding reagents or kits described herein may be comprised with a lipid carrier.

### 10. KITS COMPRISING MULTIMERIC BARCODING REAGENTS AND ADAPTER OLIGONUCLEOTIDES

The invention further provides kits comprising one or more of the components defined herein. The invention also provides kits specifically adapted for performing any of the methods defined herein.

The invention further provides a kit for labelling a target nucleic acid, wherein the kit comprises: (a) a multimeric barcoding reagent comprising (i) first and second barcode molecules linked together (i.e. a multimeric barcode molecule), wherein each of the barcode molecules comprises a nucleic acid sequence comprising, optionally in the 5′ to 3′ direction, an adapter region and a barcode region, and (ii) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule; and (b) first and second adapter oligonucleotides, wherein the first adapter oligonucleotide comprises, optionally in the 5′ to 3′ direction, an adapter region capable of annealing to the adapter region of the first barcode molecule and a target region capable of annealing or ligating to a first sub-sequence of the target nucleic acid, and wherein the second adapter oligonucleotide comprises, optionally in the 5′ to 3′ direction, an adapter region capable of annealing to the adapter region of the second barcode molecule and a target region capable of annealing or ligating to a second sub-sequence of the target nucleic acid.

The invention further provides a kit for labelling a target nucleic acid, wherein the kit comprises: (a) a multimeric barcoding reagent comprising (i) first and second barcode molecules linked together (i.e. a multimeric barcode molecule), wherein each of the barcode molecules comprises a nucleic acid sequence comprising an adapter region and a barcode region, and (ii) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule; and (b) first and second adapter oligonucleotides, wherein the first adapter oligonucleotide comprises an adapter region capable of annealing to the adapter region of the first barcode molecule and a target region capable of ligating to a first sub-sequence of the target nucleic acid, and wherein the second adapter oligonucleotide comprises an adapter region capable of annealing to the adapter region of the second barcode molecule and a target region capable of ligating to a second sub-sequence of the target nucleic acid.

The invention further provides a kit for labelling a target nucleic acid, wherein the kit comprises: (a) a multimeric barcoding reagent comprising (i) first and second barcode molecules linked together (i.e. a multimeric barcode molecule), wherein each of the barcode molecules comprises a nucleic acid sequence comprising in the 5′ to 3′ direction an adapter region and a barcode region, and (ii) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule; and (b) first and second adapter oligonucleotides, wherein the first adapter oligonucleotide comprises in the 5′ to 3′ direction an adapter region capable of annealing to the adapter region of the first barcode molecule and a target region capable of annealing to a first sub-sequence of the target nucleic acid, and wherein the second adapter oligonucleotide comprises in the 5′ to 3′ direction an adapter region capable of annealing to the adapter region of the second barcode molecule and a target region capable of annealing to a second sub-sequence of the target nucleic acid.

The invention further provides a kit for labelling a target nucleic acid, wherein the kit comprises: (a) a multimeric barcoding reagent comprising (i) first and second barcode molecules linked together (i.e. a multimeric barcode molecule), wherein each of the barcode molecules comprises a nucleic acid sequence comprising, optionally in the 5′ to 3′ direction, an adapter region and a barcode region, and (ii) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule; and (b) first and second adapter oligonucleotides, wherein the first adapter oligonucleotide comprises an adapter region capable of annealing to the adapter region of the first barcode molecule and capable of ligating to a first sub-sequence of the target nucleic acid, and wherein the second adapter oligonucleotide comprises an adapter region capable of annealing to the adapter region of the second barcode molecule and capable of ligating to a second sub-sequence of the target nucleic acid.

Each adapter oligonucleotide may consist essentially of or consist of an adapter region. Each adapter oligonucleotide may not comprise a target region.

Preferably, the adapter region of the first adapter oligonucleotide comprises a sequence that is complementary to and capable of annealing to the adapter region of the first barcode molecule and the adapter region of the second adapter oligonucleotide comprises a sequence that is complementary to and capable of annealing to the adapter region of the second barcode molecule. The complementary sequence of each adapter oligonucleotide may be at least 5, at least 10, at least 15, at least 20, at least 25, at least 50 or at least 100 contiguous nucleotides.

The target regions of the adapter oligonucleotides may not be capable of annealing to the multimeric barcode molecule(s)). The target regions of the adapter oligonucleotides may be non-complementary to the multimeric barcode molecule(s).

The target regions of each adapter oligonucleotide may comprise different sequences. Each target region may comprise a sequence capable of annealing to only a single sub-sequence of a target nucleic acid within a sample of nucleic acids. Each target region may comprise one or more random, or one or more degenerate, sequences to enable the target region to anneal to more than one sub-sequence of a target nucleic acid. Each target region may comprise at least 5, at least 10, at least 15, at least 20, at least 25, at least 50 or at least 100 nucleotides. Preferably, each target region comprises at least 5 nucleotides. Each target region may comprise 5 to 100 nucleotides, 5 to 10 nucleotides, 10 to 20 nucleotides, 20 to 30 nucleotides, 30 to 50 nucleotides, 50 to 100 nucleotides, 10 to 90 nucleotides, 20 to 80 nucleotides, 30 to 70 nucleotides or 50 to 60 nucleotides. Preferably, each target region comprises 30 to 70 nucleotides. Preferably each target region comprises deoxyribonucleotides, optionally all of the nucleotides in a target region are deoxyribonucleotides. One or more of the deoxyribonucleotides may be a modified deoxyribonucleotide (e.g. a deoxyribonucleotide modified with a biotin moiety or a deoxyuracil nucleotide). Each target region may comprise one or more universal bases (e.g. inosine), one or modified nucleotides and/or one or more nucleotide analogues.

The target regions may be used to anneal the adapter oligonucleotides to sub-sequences of target nucleic acids, and then may be used as primers for a primer-extension reaction or an amplification reaction e.g. a polymerase chain reaction. Alternatively, the target regions may be used to ligate the adapter oligonucleotides to sub-sequences of target nucleic acids. The target region may be at the 5′ end of an adapter oligonucleotide. Such a target region may be phosphorylated. This may enable the 5′ end of the target region to be ligated to the 3′ end of a sub-sequence of a target nucleic acid.

The adapter oligonucleotides may comprise a linker region between the adapter region and the target region. The linker region may comprise one or more contiguous nucleotides that are not annealed to the first and second barcode molecules (i.e. the multimeric barcode molecule) and are non-complementary to the subsequences of the target nucleic acid. The linker may comprise 1 to 100, 5 to 75, 10 to 50, 15 to 30 or 20 to 25 non-complementary nucleotides. Preferably, the linker comprises 15 to 30 non-complementary nucleotides. The use of such a linker region enhances the efficiency of the barcoding reactions performed using the kits described herein.

Each of the components of the kit may take any of the forms defined herein.

The multimeric barcoding reagent(s) and adapter oligonucleotides may be provided in the kit as physically separated components.

The kit may comprise: (a) a multimeric barcoding reagent comprising at least 5, at least 10, at least 20, at least 25, at least 50, at least 75 or at least 100 barcode molecules linked together, wherein each barcode molecule is as defined herein; and (b) an adapter oligonucleotide capable of annealing to each barcode molecule, wherein each adapter oligonucleotide is as defined herein.

FIG. 2 shows a kit comprising a multimeric barcoding reagent and adapter oligonucleotides for labelling a target nucleic acid. In more detail, the kit comprises first (D1, E1, and F1) and second (D2, E2, and F2) barcode molecules, with each incorporating a barcode region (E1 and E2) and also a 5′ adapter region (F1 and F2). These first and second barcode molecules are linked together, in this embodiment by a connecting nucleic acid sequence(S).

The kit further comprises first (A1 and B1) and second (A2 and B2) barcoded oligonucleotides, which each comprise a barcode region (B1 and B2), as well as 5′ regions (A1 and A2). The 5′ region of each barcoded oligonucleotide is complementary to, and thus may be annealed to, the 3′ regions of the barcode molecules (D1 and D2). The barcode regions (B1 and B2) are complementary to, and thus may be annealed to, the barcode regions (E1 and E2) of the barcode molecules.

The kit further comprises first (C1 and G1) and second (C2 and G2) adapter oligonucleotides, wherein each adapter oligonucleotide comprises an adapter region (C1 and C2) that is complementary to, and thus able to anneal to, the 5′ adapter region of a barcode molecule (F1 and F2). These adapter oligonucleotides may be synthesised to include a 5′-terminal phosphate group. Each adapter oligonucleotide also comprises a target region (G1 and G2), which may be used to anneal the barcoded-adapter oligonucleotides (A1, B1, C1 and G1, and A2, B2, C2 and G2) to target nucleic acids, and then may be used as primers for a primer-extension reaction or a polymerase chain reaction.

The kit may comprise a library of two or more multimeric barcoding reagents, wherein each multimeric barcoding reagent is as defined herein, and adapter oligonucleotides for each of the multimeric barcoding reagents, wherein each adapter oligonucleotide is as defined herein. The barcode regions of the first and second barcoded oligonucleotides of the first multimeric barcoding reagent are different to the barcode regions of the first and second barcoded oligonucleotides of the second multimeric barcoding reagent.

The kit may comprise a library comprising at least 5, at least 10, at least 20, at least 25, at least 50, at least 75, at least 100, at least 250, at least 500, at least 10³, at least 10⁴, at least 10⁵, at least 10⁶, at least 10⁷, at least 10⁸ or at least 10⁹ multimeric barcoding reagents as defined herein. Preferably, the kit comprises a library comprising at least 10 multimeric barcoding reagents as defined herein. The kit may further comprise adapter oligonucleotides for each of the multimeric barcoding reagents, wherein each adapter oligonucleotide may take the form of any of the adapter oligonucleotides defined herein. Preferably, the barcode regions of the first and second barcoded oligonucleotides of each multimeric barcoding reagent are different to the barcode regions of the barcoded oligonucleotides of at least 9 other multimeric barcoding reagents in the library.

The barcode regions of the first and second barcoded oligonucleotides of each multimeric barcoding reagent may be different to the barcode regions of the barcoded oligonucleotides of at least 4, at least 9, at least 19, at least 24, at least 49, at least 74, at least 99, at least 249, at least 499, at least 999 (i.e. 10³-1), at least 10⁴-1, at least 10⁵-1, at least 10⁶-1, at least 10⁷-1, at least 10⁸-1 or at least 10⁹-1 other multimeric barcoding reagents in the library. The barcode regions of the first and second barcoded oligonucleotides of each multimeric barcoding reagent may be different to the barcode regions of the barcoded oligonucleotides of all of the other multimeric barcoding reagents in the library. Preferably, the barcode regions of the first and second barcoded oligonucleotides of each multimeric barcoding reagent are different to the barcode regions of the barcoded oligonucleotides of at least 9 other multimeric barcoding reagents in the library.

The barcode regions of the barcoded oligonucleotides of each multimeric barcoding reagent may be different to the barcode regions of the barcoded oligonucleotides of at least 4, at least 9, at least 19, at least 24, at least 49, at least 74, at least 99, at least 249, at least 499, at least 999 (i.e. 10³-1), at least 10⁴-1, at least 10⁵-1, at least 10⁶-1, at least 10⁷-1, at least 10⁸-1 or at least 10⁹-1 other multimeric barcoding reagents in the library. The barcode regions of the barcoded oligonucleotides of each multimeric barcoding reagent may be different to the barcode regions of the barcoded oligonucleotides of all of the other multimeric barcoding reagents in the library. Preferably, the barcode regions of the barcoded oligonucleotides of each multimeric barcoding reagent are different to the barcode regions of the barcoded oligonucleotides of at least 9 other multimeric barcoding reagents in the library

The invention provides a kit for labelling a target nucleic acid for sequencing, wherein the kit comprises: (a) a library of multimeric barcoding reagents comprising at least 10 multimeric barcoding reagents, wherein each multimeric barcoding reagent comprises: (i) first and second barcode molecules comprised within a (single) nucleic acid molecule, wherein each of the barcode molecules comprises a nucleic acid sequence comprising, optionally in the 5′ to 3′ direction, an adapter region and a barcode region, and (ii) first and second barcoded oligonucleotides, wherein the first barcoded oligonucleotide comprises a barcode region complementary and annealed to the barcode region of the first barcode molecule, and wherein the second barcoded oligonucleotide comprises a barcode region complementary and annealed to the barcode region of the second barcode molecule; and (b) first and second adapter oligonucleotides for each of the multimeric barcoding reagents, wherein the first adapter oligonucleotide comprises, optionally in the 5′ to 3′ direction, an adapter region capable of annealing to the adapter region of the first barcode molecule and a target region capable of annealing or ligating to a first sub-sequence of the target nucleic acid, and wherein the second adapter oligonucleotide comprises, optionally in the 5′ to 3′ direction, an adapter region capable of annealing to the adapter region of the second barcode molecule and a target region capable of annealing or ligating to a second sub-sequence of the target nucleic acid.

### 11. KITS COMPRISING MULTIMERIC BARCODING REAGENTS, ADAPTER OLIGONUCLEOTIDES AND EXTENSION PRIMERS

The invention further provides a kit for labelling a target nucleic acid for sequencing, wherein the kit comprises: (a) a multimeric barcode molecule comprising first and second barcode molecules linked together, wherein each of the barcode molecules comprises a nucleic acid sequence comprising, optionally in the 5′ to 3′ direction, an adapter region, a barcode region, and a priming region; (b) first and second extension primers for the multimeric barcode molecule, wherein the first extension primer comprises a sequence capable of annealing to the priming region of the first barcode molecule, and wherein the second extension primer comprises a sequence capable of annealing to the priming region of the second barcode molecule; and (c) first and second adapter oligonucleotides for the multimeric barcode molecule, wherein the first adapter oligonucleotide comprises, optionally in the 5′ to 3′ direction, an adapter region capable of annealing to the adapter region of the first barcode molecule and a target region capable of annealing or ligating to a first sub-sequence of the target nucleic acid, and wherein the second adapter oligonucleotide comprises, optionally in the 5′ to 3′ direction, an adapter region capable of annealing to the adapter region of the second barcode molecule and a target region capable of annealing or ligating to a second sub-sequence of the target nucleic acid.

The invention further provides a kit for labelling a target nucleic acid for sequencing, wherein the kit comprises: (a) a multimeric barcode molecule comprising first and second barcode molecules linked together, wherein each of the barcode molecules comprises a nucleic acid sequence comprising, optionally in the 5′ to 3′ direction, an adapter region, a barcode region, and a priming region; (b) first and second extension primers for the multimeric barcode molecule, wherein the first extension primer comprises a sequence capable of annealing to the priming region of the first barcode molecule, and wherein the second extension primer comprises a sequence capable of annealing to the priming region of the second barcode molecule; and (c) first and second adapter oligonucleotides for the multimeric barcode molecule, wherein the first adapter oligonucleotide comprises an adapter region capable of annealing to the adapter region of the first barcode molecule and capable of ligating to a first sub-sequence of the target nucleic acid, and wherein the second adapter oligonucleotide comprises an adapter region capable of annealing to the adapter region of the second barcode molecule and capable of ligating to a second sub-sequence of the target nucleic acid.

Each adapter oligonucleotide may consist essentially of or consist of an adapter region.

The components of the kit may take any of the forms described herein.

Preferably, the first extension primer comprises a sequence that is complementary to and capable of annealing to the priming region of the first barcode molecule and the second extension primer comprises a sequence that is complementary to and capable of annealing to the priming region of the second barcode molecule. The complementary sequence of each extension primer may be at least 5, at least 10, at least 15, at least 20, at least 25, at least 50 or at least 100 contiguous nucleotides.

The first and second extension primers may be capable of being extended using the barcode regions of the first and second barcode molecules as templates to produce first and second barcoded oligonucleotides, wherein the first barcoded oligonucleotide comprises a sequence complementary to the barcode region of the first barcode molecule and the second barcoded oligonucleotide comprises a sequence complementary to the barcode region of the second barcode molecule.

The first and second extension primers may be identical in sequence. Alternatively, the first and second extension primers may be different in sequence.

The first and/or second extension primers may further comprise one or more regions with nucleic acid sequences that are not complementary to the first barcode molecule and second barcode molecule, respectively. Optionally, such a non-complementary region may include a binding site for one or more amplification primers. Optionally, such a non-complementary region may be positioned within the 5′ region of the molecule. Optionally, the first and second extension primers may comprise a terminal 5′ phosphate group capable of ligating to a 3′ end of a nucleic acid molecule.

The first and/or second extension primers may further comprise one or more secondary barcode regions. Optionally, a secondary barcode region may be comprised within a region of the extension primer that is non-complementary to a barcode molecule. Optionally, a secondary barcode region may be comprised within a region of the extension primer that is between a 3′ region of the extension primer that is complementary to a barcode molecule and a 5′ region of the extension primer that comprises a binding site for an amplification primer.

A secondary barcode region may comprise a sequence of one or more nucleotides, wherein sequences of the secondary barcode regions of the first extension primer and the second extension primer are different. Optionally, said one or more nucleotides may comprise random or degenerate nucleotides. Optionally, said one or more nucleotides may comprise different but non-random nucleotides. Any secondary barcode region may comprise at least 2, at least 3, at least 5, at least 10, at least 15, at least 20, or at least 30 nucleotides. Any secondary barcode region may comprise a contiguous sequence of barcode oligonucleotides, or may comprise two or more different segments separated by at least one non-barcode or invariant nucleotide. Optionally, any secondary barcode region may comprise a unique molecular identifier (UMI).

The kit may comprise a library of two or more multimeric barcode molecules, wherein each multimeric barcode molecule is as defined herein, and first and second extension primers, and first and second adapter oligonucleotides, for each of the multimeric barcode molecule. The extension primers and adapter oligonucleotides may take any of the forms described herein. The barcode regions of the first and second barcode molecules of the first multimeric barcode molecule are different to the barcode regions of the first and second barcode molecules of the second multimeric barcode molecule.

The kit may comprise a library comprising at least 5, at least 10, at least 20, at least 25, at least 50, at least 75, at least 100, at least 250, at least 500, at least 10³, at least 10⁴, at least 10⁵, at least 10⁶, at least 10⁷, at least 10⁸ or at least 10⁹ multimeric barcode molecules as defined herein. Preferably, the kit comprises a library comprising at least 10 multimeric barcode molecules as defined herein. The kit may further comprise extension primers and/or adapter oligonucleotides for each of the multimeric barcode molecules. The extension primers and adapter oligonucleotides may take any of the forms described herein. Preferably, the barcode regions of the first and second barcode molecules of each multimeric barcode molecule are different to the barcode regions of the barcode molecules of at least 9 other multimeric barcode molecules in the library.

The barcode regions of the first and second barcode molecules of each multimeric barcode molecule may be different to the barcode regions of the barcoded molecules of at least 4, at least 9, at least 19, at least 24, at least 49, at least 74, at least 99, at least 249, at least 499, at least 999 (i.e. 10³-1), at least 10⁴-1, at least 10⁵-1, at least 10⁶-1, at least 10⁷-1, at least 10⁸-1 or at least 10⁹-1 other multimeric barcode molecules in the library. The barcode regions of the first and second barcode molecules of each multimeric barcode molecule may be different to the barcode regions of the barcode molecules of all of the other multimeric barcode molecules in the library. Preferably, the barcode regions of the first and second barcode molecules of each multimeric barcode molecule are different to the barcode regions of the barcode molecules of at least 9 other multimeric barcode molecules in the library.

The barcode regions of the barcode molecules of each multimeric barcode molecule may be different to the barcode regions of the barcode molecules of at least 4, at least 9, at least 19, at least 24, at least 49, at least 74, at least 99, at least 249, at least 499, at least 999 (i.e. 10³-1), at least 10⁴-1, at least 10⁵-1, at least 10⁶-1, at least 10⁷-1, at least 10⁸-1 or at least 10⁹-1 other multimeric barcode molecules in the library. The barcode regions of the barcode molecules of each multimeric barcode molecules may be different to the barcode regions of the barcode molecules of all of the other multimeric barcode molecules in the library. Preferably, the barcode regions of the barcode molecules of each multimeric barcode molecule are different to the barcode regions of the barcode molecules of at least 9 other multimeric barcode molecules in the library.

The invention further provides a kit for labelling a target nucleic acid for sequencing, wherein the kit comprises: (a) a library of multimeric barcode molecules comprising at least 10 multimeric barcode molecules, each multimeric barcode molecule comprising first and second barcode molecules comprised within a (single) nucleic acid molecule, wherein each of the barcode molecules comprises a nucleic acid sequence comprising, optionally in the 5′ to 3′ direction, an adapter region, a barcode region, and a priming region, and wherein the barcode regions of the first and second barcode molecules of each multimeric barcode molecule are different to the barcode regions of at least 9 other multimeric barcode molecules in the library; (b) first and second extension primers for each of the multimeric barcode molecules, wherein the first extension primer comprises a sequence capable of annealing to the priming region of the first barcode molecule, and wherein the second extension primer comprises a sequence capable of annealing to the priming region of the second barcode molecule; and (c) first and second adapter oligonucleotides for each of the multimeric barcode molecules, wherein the first adapter oligonucleotide comprises, optionally in the 5′ to 3′ direction, an adapter region capable of annealing to the adapter region of the first barcode molecule and a target region capable of annealing or ligating to a first sub-sequence of the target nucleic acid, and wherein the second adapter oligonucleotide comprises, optionally in the 5′ to 3′ direction, an adapter region capable of annealing to the adapter region of the second barcode molecule and a target region capable of annealing or ligating to a second sub-sequence of the target nucleic acid.

### 12. METHODS OF PREPARING A NUCLEIC ACID SAMPLE FOR SEQUENCING

The methods of preparing a nucleic acid sample for sequencing may comprise (i) contacting the nucleic acid sample with a multimeric barcoding reagent comprising first and second barcode regions linked together, wherein each barcode region comprises a nucleic acid sequence, and (ii) appending barcode sequences to first and second sub-sequences of a target nucleic acid to produce first and second different barcoded target nucleic acid molecules, wherein the first barcoded target nucleic acid molecule comprises the nucleic acid sequence of the first barcode region and the second barcoded target nucleic acid molecule comprises the nucleic acid sequence of the second barcode region.

In methods in which the multimeric barcoding reagent comprises first and second barcoded oligonucleotides linked together, the barcode sequences may be appended to first and second sub-sequences of the target nucleic acid by any of the methods described herein.

The first and second barcoded oligonucleotides may be ligated to the first and second sub-sequences of the target nucleic acid to produce the first and second different barcoded target nucleic acid molecules. Optionally, prior to the ligation step, the method comprises appending first and second coupling sequences to the target nucleic acid, wherein the first and second coupling sequences are the first and second sub-sequences of the target nucleic acid to which the first and second barcoded oligonucleotides are ligated.

The first and second barcoded oligonucleotides may be annealed to the first and second sub-sequences of the target nucleic acid extended to produce the first and second different barcoded target nucleic acid molecules. Optionally, prior to the annealing step, the method comprises appending first and second coupling sequences to the target nucleic acid, wherein the first and second coupling sequences are the first and second sub-sequences of the target nucleic acid to which the first and second barcoded oligonucleotides are annealed.

The first and second barcoded oligonucleotides may be annealed at their 5′ ends to the first and second sub-sequences of the target nucleic acid and first and second target primers may be annealed to third and fourth sub-sequences of the target nucleic acid, respectively, wherein the third subsequence is 3′ of the first subsequence and wherein the fourth sub-sequence is 3′ of the second subsequence. The method further comprises extending the first target primer using the target nucleic acid as template until it reaches the first sub-sequence to produce a first extended target primer, and extending the second target primer using the target nucleic acid as template until it reaches the second sub-sequence to produce a second extended target primer, and ligating the 3′ end of the first extended target primer to the 5′ end of the first barcoded oligonucleotide to produce a first barcoded target nucleic acid molecule, and ligating the 3′ end of the second extended target primer to the 5′ end of the second barcoded oligonucleotide to produce a second barcoded target nucleic acid molecule, wherein the first and second barcoded target nucleic acid molecules are different and each comprises at least one nucleotide synthesised from the target nucleic acid as a template. Optionally, prior to either or both annealing step(s), the method comprises appending first and second, and/or third and fourth, coupling sequences to the target nucleic acid, wherein the first and second coupling sequences are the first and second sub-sequences of the target nucleic acid to which the first and second barcoded oligonucleotides are annealed, and/or wherein the third and fourth coupling sequences are the third and fourth sub-sequences of the target nucleic acid to which the first and second target primers are annealed.

As described herein, prior to annealing or ligating a multimeric hybridization molecule, multimeric barcode molecule, barcoded oligonucleotide, adapter oligonucleotide or target primer to a target nucleic acid, a coupling sequence may be appended to the target nucleic acid. The multimeric hybridization molecule, multimeric barcode molecule, barcoded oligonucleotide, adapter oligonucleotide or target primer may then be annealed or ligated to the coupling sequence.

A coupling sequence may be added to the 5′ end or 3′ end of two or more target nucleic acids of the nucleic acid sample (e.g. a FFPE DNA sample). In this method, the target regions (of the barcoded oligonucleotides) may comprise a sequence that is complementary to the coupling sequence.

A coupling sequence may be comprised within a double-stranded coupling oligonucleotide or within a single-stranded coupling oligonucleotide. A coupling oligonucleotide may be appended to the target nucleic acid by a double-stranded ligation reaction or a single-stranded ligation reaction. A coupling oligonucleotide may comprise a single-stranded 5′ or 3′ region capable of ligating to a target nucleic acid and the coupling sequence may be appended to the target nucleic acid by a single-stranded ligation reaction.

A coupling oligonucleotide may comprise a blunt, recessed, or overhanging 5′ or 3′ region capable of ligating to a target nucleic acid and the coupling sequence may be appended to the target nucleic acid a double-stranded ligation reaction.

The end(s) of a target nucleic acid may be converted into blunt double-stranded end(s) in a blunting reaction, and the coupling oligonucleotide may comprise a blunt double-stranded end, and wherein the coupling oligonucleotide may be ligated to the target nucleic acid in a blunt-end ligation reaction.

The end(s) of a target nucleic acid may be converted into blunt double-stranded end(s) in a blunting reaction, and then converted into a form with (a) single 3′ adenosine overhang(s), and wherein the coupling oligonucleotide may comprise a double-stranded end with a single 3′ thymine overhang capable of annealing to the single 3′ adenosine overhang of the target nucleic acid, and wherein the coupling oligonucleotide is ligated to the target nucleic acid in a double-stranded A/T ligation reaction

The target nucleic acid may be contacted with a restriction enzyme, wherein the restriction enzyme digests the target nucleic acid at restriction sites to create (a) ligation junction(s) at the restriction site(s), and wherein the coupling oligonucleotide comprises an end compatible with the ligation junction, and wherein the coupling oligonucleotide is then ligated to the target nucleic acid in a double-stranded ligation reaction.

A coupling oligonucleotide may be appended via a primer-extension or polymerase chain reaction step.

A coupling oligonucleotide may be appended via a primer-extension or polymerase chain reaction step, using one or more oligonucleotide(s) that comprise a priming segment including one or more degenerate bases.

A coupling oligonucleotide may be appended via a primer-extension or polymerase chain reaction step, using one or more oligonucleotide(s) that further comprise a priming or hybridization segment specific for a particular target nucleic acid sequence.

A coupling sequence may be added by a polynucleotide tailing reaction. A coupling sequence may be added by a terminal transferase enzyme (e.g. a terminal deoxynucleotidyl transferase enzyme). A coupling sequence may be appended via a polynucleotide tailing reaction performed with a terminal deoxynucleotidyl transferase enzyme, and wherein the coupling sequence comprises at least two contiguous nucleotides of a homopolymeric sequence.

A coupling sequence may comprise a homopolymeric 3′ tail (e.g. a poly(A) tail). Optionally, in such methods, the target regions (of the barcoded oligonucleotides) comprise a complementary homopolymeric 3′ tail (e.g. a poly(T) tail).

A coupling sequence may be comprised within a synthetic transposome, and may be appended via an in vitro transposition reaction.

A coupling sequence may be appended to a target nucleic acid, and wherein a barcode oligonucleotide is appended to the target nucleic acid by at least one primer-extension step or polymerase chain reaction step, and wherein said barcode oligonucleotide comprises a region of at least one nucleotide in length that is complementary to said coupling sequence. Optionally, this region of complementarity is at the 3′ end of the barcode oligonucleotide. Optionally, this region of complementarity is at least 2 nucleotides in length, at least 5 nucleotides in length, at least 10 nucleotides in length, at least 20 nucleotides in length, or at least 50 nucleotides in length.

In methods in which an adapter oligonucleotide is appended (e.g. ligated or annealed) to a target nucleic acid, the adapter region of the adapter oligonucleotide provides a coupling sequence capable of hybridizing to the adapter region of a multimeric hybridization molecule or a multimeric barcode molecule.

The invention provides a method of preparing a nucleic acid sample for sequencing comprising the steps of: (a) appending a coupling sequence to first and second sub-sequences of a target nucleic acid; (b) contacting the nucleic acid sample with a multimeric barcoding reagent comprising first and second barcode molecules linked together, wherein each of the barcode molecules comprises a nucleic acid sequence comprising (in the 5′ to 3′ or 3′ to 5′ direction), a barcode region and an adapter region; (c) annealing the coupling sequence of the first sub-sequence to the adapter region of the first barcode molecule, and annealing the coupling sequence of the second sub-sequence to the adapter region of the second barcode molecule; and (d) appending barcode sequences to each of the at least two sub-sequences of the target nucleic acid to produce first and second different barcoded target nucleic acid molecules, wherein the first barcoded target nucleic acid molecule comprises the nucleic acid sequence of the barcode region of the first barcode molecule and the second barcoded target nucleic acid molecule comprises the nucleic acid sequence of the barcode region of the second barcode molecule.

In the method, each of the barcode molecules may comprise a nucleic acid sequence comprising, in the 5′ to 3′ direction, a barcode region and an adapter region, and step (d) may comprise extending the coupling sequence of the first sub-sequence of the target nucleic acid using the barcode region of the first barcode molecule as a template to produce a first barcoded target nucleic acid molecule, and extending the coupling sequence of the second sub-sequence of the target nucleic acid using the barcode region of the second barcode molecule as a template to produce a second barcoded target nucleic acid molecule, wherein the first barcoded target nucleic acid molecule comprises a sequence complementary to the barcode region of the first barcode molecule and the second barcoded target nucleic acid molecule comprises a sequence complementary to the barcode region of the second barcode molecule.

In the method, each of the barcode molecules may comprise a nucleic acid sequence comprising, in the 5′ to 3′ direction, an adapter region and a barcode region, and step (d) may comprise (i) annealing and extending a first extension primer using the barcode region of the first barcode molecule as a template to produce a first barcoded oligonucleotide, and annealing and extending a second extension primer using the barcode region of the second barcode molecule as a template to produce a second barcoded oligonucleotide, wherein the first barcoded oligonucleotide comprises a sequence complementary to the barcode region of the first barcode molecule and the second barcoded oligonucleotide comprises a sequence complementary to the barcode region of the second barcode molecule, (ii) ligating the 3′ end of the first barcoded oligonucleotide to the 5′ end of the coupling sequence of the first sub-sequence of the target nucleic acid to produce a first barcoded target nucleic acid molecule and ligating the 3′ end of the second barcoded oligonucleotide to the 5′ end of the coupling sequence of the second sub-sequence of the target nucleic acid to produce a second barcoded target nucleic acid molecule.

In the method, each of the barcode molecules may comprise a nucleic acid sequence comprising, in the 5′ to 3′ direction, an adapter region, a barcode region and a priming region wherein step (d) comprises (i) annealing a first extension primer to the priming region of the first barcode molecule and extending the first extension primer using the barcode region of the first barcode molecule as a template to produce a first barcoded oligonucleotide, and annealing a second extension primer to the priming region of the second barcode molecule and extending the second extension primer using the barcode region of the second barcode molecule as a template to produce a second barcoded oligonucleotide, wherein the first barcoded oligonucleotide comprises a sequence complementary to the barcode region of the first barcode molecule and the second barcoded oligonucleotide comprises a sequence complementary to the barcode region of the second barcode molecule, (ii) ligating the 3′ end of the first barcoded oligonucleotide to the 5′ end of the coupling sequence of the first sub-sequence of the target nucleic acid to produce a first barcoded target nucleic acid molecule and ligating the 3′ end of the second barcoded oligonucleotide to the 5′ end of the coupling sequence of the second sub-sequence of the target nucleic acid to produce a second barcoded target nucleic acid molecule.

The methods for preparing a nucleic acid sample for sequencing may be used to prepare a range of different nucleic acid samples for sequencing. The target nucleic acids may be DNA molecules (e.g. genomic DNA molecules) or RNA molecules (e.g. mRNA molecules). The target nucleic acids may be from any sample. For example, an individual cell (or cells), a tissue, a bodily fluid (e.g. blood, plasma and/or serum), a biopsy or a formalin-fixed paraffin-embedded (FFPE) sample.

The sample may comprise at least 10, at least 100, or at least 10³, at least 10⁴, at least 10⁵, at least 10⁶, at least 10⁷, at least 10⁸ or at least 10⁹ target nucleic acids

The target nucleic acid may be a (single) intact nucleic acid molecule of a cell or two or more co-localised fragments of a nucleic acid molecule of a cell. As used herein the term target nucleic acid refers to the nucleic acids present within cells and to copies or amplicons thereof. For example, where the target nucleic acid is genomic DNA, the term target nucleic acid means genomic DNA present in a cell and copies or amplicons thereof e.g. DNA molecules that may be prepared from the genomic DNA by a primer-extension reaction. As a further example, where the target nucleic acid is mRNA, the term target nucleic acid means mRNA present in the cell and copies or amplicons thereof e.g. cDNA synthesized from the mRNA by reverse transcription.

The method may comprise producing at least 2, at least 5, at least 10, at least 20, at least 25, at least 50, at least 75, at least 100, at least 250, at least 500, at least 10³, at least 10⁴, at least 10⁵, at least 10⁶, at least 10⁷, at least 10⁸ or at least 10⁹ different barcoded target nucleic acid molecules. Preferably, the method comprises producing at least 5 different barcoded target nucleic acid molecules.

Each barcoded target nucleic acid molecule may comprise at least 1, at least 5, at least 10, at least 25, at least 50, at least 100, at least 250, at least 500, at least 1000, at least 2000, at least 5000, or at least 10,000 nucleotides synthesised from the target nucleic acid as template. Preferably, each barcoded target nucleic acid molecule comprises at least 20 nucleotides synthesised from the target nucleic acid as template.

Alternatively, each barcoded target nucleic acid molecule may comprise at least 5, at least 10, at least 25, at least 50, at least 100, at least 250, at least 500, at least 1000, at least 2000, at least 5000, or at least 10,000 nucleotides of the target nucleic acid. Preferably, each barcoded target nucleic acid molecule comprises at least 5 nucleotides of the target nucleic acid.

A universal priming sequence may be added to the barcoded target nucleic acid molecules. This sequence may enable the subsequent amplification of at least 5, at least 10, at least 20, at least 25, at least 50, at least 75, at least 100, at least 250, at least 500, at least 10³, at least 10⁴, at least 105, at least 10⁶, at least 10⁷, at least 10⁸, or at least 10⁹ different barcoded target nucleic acid molecules using one forward primer and one reverse primer.

The method may comprise preparing two or more independent nucleic acid samples for sequencing, wherein each nucleic acid sample is prepared using a different library of multimeric barcoding reagents (or a different library of multimeric barcode molecules), and wherein the barcode regions of each library of multimeric barcoding reagents (or multimeric barcode molecules) comprise a sequence that is different to the barcode regions of the other libraries of multimeric barcoding reagents (or multimeric barcode molecules). Following the separate preparation of each of the samples for sequencing, the barcoded target nucleic acid molecules prepared from the different samples may be pooled and sequenced together. The sequence read generated for each barcoded target nucleic acid molecule may be used to identify the library of multimeric barcoding reagents (or multimeric barcode molecules) that was used in its preparation and thereby to identify the nucleic acid sample from which it was prepared.

In any method of preparing a nucleic acid sample for sequencing, the target nucleic acid molecules may be present at particular concentrations within the nucleic acid sample, for example at concentrations of at least 100 nanomolar, at least 10 nanomolar, at least 1 nanomolar, at least 100 picomolar, at least 10 picomolar, at least 1 picomolar, at least 100 femtomolar, at least 10 femtomolar, or at least 1 femtomolar. The concentrations may be 1 picomolar to 100 nanomolar, 10 picomolar to 10 nanomolar, or 100 picomolar to 1 nanomolar. Preferably, the concentrations are 10 picomolar to 1 nanomolar.

In any method of preparing a nucleic acid sample for sequencing, the multimeric barcoding reagents may be present at particular concentrations within the nucleic acid sample, for example at concentrations of at least 100 nanomolar, at least 10 nanomolar, at least 1 nanomolar, at least 100 picomolar, at least 10 picomolar, at least 1 picomolar, at least 100 femtomolar, at least 10 femtomolar, or at least 1 femtomolar. The concentrations may be 1 picomolar to 100 nanomolar, 10 picomolar to 10 nanomolar, or 100 picomolar to 1 nanomolar. Preferably, the concentrations are 1 picomolar to 100 picomolar.

In any method of preparing a nucleic acid sample for sequencing, the multimeric barcode molecules may be present at particular concentrations within the nucleic acid sample, for example at concentrations of at least 100 nanomolar, at least 10 nanomolar, at least 1 nanomolar, at least 100 picomolar, at least 10 picomolar, at least 1 picomolar, at least 100 femtomolar, at least 10 femtomolar, or at least 1 femtomolar. The concentrations may be 1 picomolar to 100 nanomolar, 10 picomolar to 10 nanomolar, or 100 picomolar to 1 nanomolar. Preferably, the concentrations are 1 picomolar to 100 picomolar.

In any method of preparing a nucleic acid sample for sequencing, the barcoded oligonucleotides may be present at particular concentrations within the nucleic acid sample, for example at concentrations of at least 100 nanomolar, at least 10 nanomolar, at least 1 nanomolar, at least 100 picomolar, at least 10 picomolar, at least 1 picomolar, at least 100 femtomolar, at least 10 femtomolar, or at least 1 femtomolar. The concentrations may be 1 picomolar to 100 nanomolar, 10 picomolar to 10 nanomolar, or 100 picomolar to 1 nanomolar. Preferably, the concentrations are 100 picomolar to 100 nanomolar.

### 13. METHODS OF PREPARING A NUCLEIC ACID SAMPLE FOR SEQUENCING USING MULTIMERIC BARCODING REAGENTS

The invention provides a method of preparing a nucleic acid sample for sequencing, wherein the method comprises the steps of: contacting the nucleic acid sample with a multimeric barcoding reagent as defined herein; annealing the target region of the first barcoded oligonucleotide to a first sub-sequence of a target nucleic acid, and annealing the target region of the second barcoded oligonucleotide to a second sub-sequence of the target nucleic acid; and extending the first and second barcoded oligonucleotides to produce first and second different barcoded target nucleic acid molecules, wherein each of the barcoded target nucleic acid molecules comprises at least one nucleotide synthesised from the target nucleic acid as a template.

In any method of preparing a nucleic acid sample for sequencing, either the nucleic acid molecules within the nucleic acid sample, and/or the multimeric barcoding reagents, may be present at particular concentrations within the solution volume, for example at concentrations of at least 100 nanomolar, at least 10 nanomolar, at least 1 nanomolar, at least 100 picomolar, at least 10 picomolar, or at least 1 picomolar. The concentrations may be 1 picomolar to 100 nanomolar, 10 picomolar to 10 nanomolar, or 100 picomolar to 1 nanomolar. Alternative higher or lower concentrations may also be used.

The method of preparing a nucleic acid sample for sequencing may comprise contacting the nucleic acid sample with a library of multimeric barcoding reagents as defined herein, and wherein: the barcoded oligonucleotides of the first multimeric barcoding reagent anneal to sub-sequences of a first target nucleic acid and first and second different barcoded target nucleic acid molecules are produced, wherein each barcoded target nucleic acid molecule comprises at least one nucleotide synthesised from the first target nucleic acid as a template; and the barcoded oligonucleotides of the second multimeric barcoding reagent anneal to sub-sequences of a second target nucleic acid and first and second different barcoded target nucleic acid molecules are produced, wherein each barcoded target nucleic acid molecule comprises at least one nucleotide synthesised from the second target nucleic acid as a template.

In the method the barcoded oligonucleotides may be isolated from the nucleic acid sample after annealing to the sub-sequences of the target nucleic acid and before the barcoded target nucleic acid molecules are produced. Optionally, the barcoded oligonucleotides are isolated by capture on a solid support through a streptavidin-biotin interaction.

Additionally or alternatively, the barcoded target nucleic acid molecules may be isolated from the nucleic acid sample. Optionally, the barcoded target nucleic acid molecules are isolated by capture on a solid support through a streptavidin-biotin interaction.

The step of extending the barcoded oligonucleotides may be performed while the barcoded oligonucleotides are annealed to the barcode molecules.

FIG. 3 shows a method of preparing a nucleic acid sample for sequencing, in which a multimeric barcoding reagent defined herein (for example, as illustrated in FIG. 1) is used to label and extend two or more nucleic acid sub-sequences in a nucleic acid sample. In this method, a multimeric barcoding reagent is synthesised which incorporates at least a first (A1, B1, C1, and G1) and a second (A2, B2, C2, and G2) barcoded oligonucleotide, which each comprise both a barcode region (B1 and B2) and a target region (G1 and G2 respectively).

A nucleic acid sample comprising a target nucleic acid is contacted or mixed with the multimeric barcoding reagent, and the target regions (G1 and G2) of two or more barcoded oligonucleotides are allowed to anneal to two or more corresponding sub-sequences within the target nucleic acid (H1 and H2). Following the annealing step, the first and second barcoded oligonucleotides are extended (e.g. with the target regions serving as primers for a polymerase) into the sequence of the target nucleic acid, such that at least one nucleotide of a sub-sequence is incorporated into the extended 3′ end of each of the barcoded oligonucleotides. This method creates barcoded target nucleic acid molecules, wherein two or more sub-sequences from the target nucleic acid are labeled by a barcoded oligonucleotide.

Alternatively, the method may further comprise the step of dissociating the barcoded oligonucleotides from the barcode molecules before annealing the target regions of the barcoded oligonucleotides to sub-sequences of the target nucleic acid.

FIG. 4 shows a method of preparing a nucleic acid sample for sequencing, in which a multimeric barcoding reagent described herein (for example, as illustrated in FIG. 1) is used to label and extend two or more nucleic acid sub-sequences in a nucleic acid sample, but wherein the barcoded oligonucleotides from the multimeric barcoding reagent are dissociated from the barcode molecules prior to annealing to (and extension of) target nucleic acid sequences. In this method, a multimeric barcoding reagent is synthesised which incorporates at least a first (A1, B1, C1, and G1) and a second (A2, B2, C2, and G2) barcoded oligonucleotide, which each comprise a barcode region (B1 and B2) and a target region (G1 and G2).

A nucleic acid sample comprising a target nucleic acid is contacted with the multimeric barcoding reagent, and then the barcoded oligonucleotides are dissociated from the barcode molecules.

This step may be achieved, for example, through exposing the reagent to an elevated temperature (e.g. a temperature of at least 35° C., at least 40° C., at least 45° C., at least 50° C., at least 55° C., at least 60° C., at least 65° C., at least 70° C., at least 75° C., at least 80° C., at least 85° C., or at least 90° C.) or through a chemical denaturant, or a combination thereof. This step may also denature double-stranded nucleic acids within the sample itself. The barcoded oligonucleotides may then be allowed to for diffuse for a certain amount of time (e.g. at least 5 seconds, at least 15 seconds, at least 30 seconds, at least 60 seconds, at least 2 minutes, at least 5 minutes, at least 15 minutes, at least 30 minutes, or at least 60 minutes) (and correspondingly, to diffuse a certain physical distance within the sample).

The conditions of the reagent-sample mixture may then be changed to allow the target regions (e.g. G1 and G2) of two or more barcoded oligonucleotides to anneal to two or more corresponding sub-sequences within the target nucleic acid (e.g. H1 and H2). This could comprise, for example, lowering the temperature of the solution to allow annealing (for example, lowering the temperature to less than 90° C., less than 85° C., less than 70° C., less than 65° C., less than 60° C., less than 55° C., less than 50° C., less than 45° C., less than 40° C., less than 35° C., less than 30° C., less than 25° C., or less than 20° C.). Following this annealing step (or for example, following a purification/preparation step), the first and second barcoded oligonucleotides are extended (e.g. with the target regions serving as primers for a polymerase) into the sequence of the target nucleic acid, such that at least one nucleotide of a sub-sequence is incorporated into the extended 3′ end of each of the barcoded oligonucleotides.

This method creates barcoded target nucleic acid molecules wherein two or more sub-sequences from the nucleic acid sample are labeled by a barcoded oligonucleotide. In addition, the step of dissociating the barcoded oligonucleotides and allowing them to diffuse through the sample holds advantages for particular types of samples. For example, cross-linked nucleic acid samples (e.g. formalin-fixed, paraffin-embedded (FFPE) samples) may be amenable to the diffusion of relatively small, individual barcoded oligonucleotides. This method may allow labeling of nucleic acid samples with poor accessibility (e.g. FFPE samples) or other biophysical properties e.g. where target nucleic acid sub-sequences are physically far away from each other.

A universal priming sequence may be added to the barcoded target nucleic acid molecules. This sequence may enable the subsequent amplification of at least 5, at least 10, at least 20, at least 25, at least 50, at least 75, at least 100, at least 250, at least 500, at least 10³, at least 10⁴, at least 10⁵, at least 10⁶, at least 10⁷, at least 10⁸, or at least 10⁹ different barcoded target nucleic acid molecules using one forward primer and one reverse primer.

Prior to contacting the nucleic acid sample with a multimeric barcoding reagent, or library of multimeric barcoding reagents, as defined herein, a coupling sequence may be added to the 5′ end or 3′ end of two or more target nucleic acids of the nucleic acid sample (e.g. a FFPE DNA sample). In this method, the target regions may comprise a sequence that is complementary to the coupling sequence. The coupling sequence may comprise a homopolymeric 3′ tail (e.g. a poly(A) tail). The coupling sequence may be added by a terminal transferase enzyme. In the method in which the coupling sequence comprises a poly(A) tail, the target regions may comprise a poly(T) sequence. Such coupling sequences may be added following a high-temperature incubation of the nucleic acid sample, to denature the nucleic acids contained therein prior to adding a coupling sequence.

Alternatively, a coupling sequence could be added by digestion of a target nucleic acid sample (e.g. an FFPE DNA sample) with a restriction enzyme, in which case a coupling sequence may be comprised of one or more nucleotides of a restriction enzyme recognition sequence. In this case, a coupling sequence may be at least partially double-stranded, and may comprise a blunt-ended double-stranded DNA sequence, or a sequence with a 5′ overhang region of 1 or more nucleotides, or a sequence with a 3′ overhang region of 1 or more nucleotides. In these cases, target regions in multimeric barcoding reagents may then comprise sequences that are either double-stranded and blunt-ended (and thus able to ligate to blunt-ended restriction digestion products), or the target regions may contain 5′ or 3′ overhang sequences of 1 or more nucleotides, which make them cohesive (and thus able to anneal with and ligate to) against said restriction digestion products.

The method may comprise preparing two or more independent nucleic acid samples for sequencing, wherein each nucleic acid sample is prepared using a different library of multimeric barcoding reagents (or a different library of multimeric barcode molecules), and wherein the barcode regions of each library of multimeric barcoding reagents (or multimeric barcode molecules) comprise a sequence that is different to the barcode regions of the other libraries of multimeric barcoding reagents (or multimeric barcode molecules). Following the separate preparation of each of the samples for sequencing, the barcoded target nucleic acid molecules prepared from the different samples may be pooled and sequenced together. The sequence read generated for each barcoded target nucleic acid molecule may be used to identify the library of multimeric barcoding reagents (or multimeric barcode molecules) that was used in its preparation and thereby to identify the nucleic acid sample from which it was prepared.

The invention provides a method of preparing a nucleic acid sample for sequencing, wherein the method comprises the steps of: (a) contacting the nucleic acid sample with a multimeric barcoding reagent, wherein each barcoded oligonucleotide comprises in the 5′ to 3′ direction a target region and a barcode region, and first and second target primers; (b) annealing the target region of the first barcoded oligonucleotide to a first sub-sequence of a target nucleic acid and annealing the target region of the second barcoded oligonucleotide to a second sub-sequence of the target nucleic acid; (c) annealing the first target primer to a third sub-sequence of the target nucleic acid, wherein the third sub-sequence is 3′ of the first sub-sequence, and annealing the second target primer to a fourth sub-sequence of the target nucleic acid, wherein the fourth sub-sequence is 3′ of the second sub-sequence; (d) extending the first target primer using the target nucleic acid as template until it reaches the first sub-sequence to produce a first extended target primer, and extending the second target primer using the target nucleic acid as template until it reaches the second sub-sequence to produce a second extended target primer; and (e) ligating the 3′ end of the first extended target primer to the 5′ end of the first barcoded oligonucleotide to produce a first barcoded target nucleic acid molecule, and ligating the 3′ end of the second extended target primer to the 5′ end of the second barcoded oligonucleotide to produce a second barcoded target nucleic acid molecule, wherein the first and second barcoded target nucleic acid molecules are different, and wherein each of the barcoded target nucleic acid molecules comprises at least one nucleotide synthesised from the target nucleic acid as a template.

In the method, steps (b) and (c) may be performed at the same time.

### 14. METHODS OF PREPARING A NUCLEIC ACID SAMPLE FOR SEQUENCING USING MULTIMERIC BARCODING REAGENTS AND ADAPTER OLIGONUCLEOTIDES

The methods provided below may be performed with any of the kits defined herein.

The invention further provides a method of preparing a nucleic acid sample for sequencing, wherein the method comprises the steps of: (a) contacting the nucleic acid sample with a first and second adapter oligonucleotide as defined herein; (b) annealing or ligating the first adapter oligonucleotide to a first sub-sequence of a target nucleic acid, and annealing or ligating the second adapter oligonucleotide to a second sub-sequence of the target nucleic acid; (c) contacting the nucleic acid sample with a multimeric barcoding reagent as defined herein; (d) annealing the adapter region of the first adapter oligonucleotide to the adapter region of the first barcode molecule, and annealing the adapter region of the second adapter oligonucleotide to the adapter region of the second barcode molecule; and (e) ligating the 3′ end of the first barcoded oligonucleotide to the 5′ end of the first adapter oligonucleotide to produce a first barcoded-adapter oligonucleotide and ligating the 3′ end of the second barcoded oligonucleotide to the 5′ end of the second adapter oligonucleotide to produce a second barcoded-adapter oligonucleotide.

The invention further provides a method of preparing a nucleic acid sample for sequencing, wherein the method comprises the steps of: (a) contacting the nucleic acid sample with a first and second adapter oligonucleotide as defined herein; (b) the first adapter oligonucleotide to a first sub-sequence of a target nucleic acid, and ligating the second adapter oligonucleotide to a second sub-sequence of the target nucleic acid; (c) contacting the nucleic acid sample with a multimeric barcoding reagent as defined herein; (d) annealing the adapter region of the first adapter oligonucleotide to the adapter region of the first barcode molecule, and annealing the adapter region of the second adapter oligonucleotide to the adapter region of the second barcode molecule; and (e) extending the first adapter oligonucleotide using the barcode region of the first barcode molecule as a template to produce a first barcoded target nucleic acid molecule, and extending the second adapter oligonucleotide using the barcode region of the second barcode molecule as a template to produce a second barcoded target nucleic acid molecule, wherein the first barcoded target nucleic acid molecule comprises a sequence complementary to the barcode region of the first barcode molecule and the second barcoded target nucleic acid molecule comprises a sequence complementary to the barcode region of the second barcode molecule.

The invention further provides a method of preparing a nucleic acid sample for sequencing, wherein the method comprises the steps of: (a) contacting the nucleic acid sample with a first and second adapter oligonucleotide as defined herein; (b) annealing the target region of the first adapter oligonucleotide to a first sub-sequence of a target nucleic acid, and annealing the target region of the second adapter oligonucleotide to a second sub-sequence of the target nucleic acid; (c) contacting the nucleic acid sample with a multimeric barcoding reagent as defined herein; (d) annealing the adapter region of the first adapter oligonucleotide to the adapter region of the first barcode molecule, and annealing the adapter region of the second adapter oligonucleotide to the adapter region of the second barcode molecule; and (e) ligating the 3′ end of the first barcoded oligonucleotide to the 5′ end of the first adapter oligonucleotide to produce a first barcoded-adapter oligonucleotide and ligating the 3′ end of the second barcoded oligonucleotide to the 5′ end of the second adapter oligonucleotide to produce a second barcoded-adapter oligonucleotide.

In the method the first and second barcoded-adapter oligonucleotides may be extended to produce first and second different barcoded target nucleic acid molecules each of which comprises at least one nucleotide synthesised from the target nucleic acid as a template.

Alternatively, the first and second adapter oligonucleotides may be extended to produce first and second different target nucleic acid molecules each of which comprises at least one nucleotide synthesised from the target nucleic acid as a template. In this method, step (f) produces a first barcoded target nucleic acid molecule (i.e. the first barcoded oligonucleotide ligated to the extended first adapter oligonucleotide) and a second barcoded target nucleic acid molecule (i.e. the second barcoded oligonucleotide ligated to the extended second adapter oligonucleotide).

The step of extending the adapter oligonucleotides may be performed before step (c), before step (d) and/or before step (e), and the first and second adapter oligonucleotides may remain annealed to the first and second barcode molecules until after step (e).

The method may be performed using a library of multimeric barcoding reagents as defined herein and an adapter oligonucleotide as defined herein for each of the multimeric barcoding reagents.

Preferably, the barcoded-adapter oligonucleotides of the first multimeric barcoding reagent anneal to sub-sequences of a first target nucleic acid and first and second different barcoded target nucleic acid molecules are produced, wherein each barcoded target nucleic acid molecule comprises at least one nucleotide synthesised from the first target nucleic acid as a template; and the barcoded-adapter oligonucleotides of the second multimeric barcoding reagent anneal to sub-sequences of a second target nucleic acid and first and second different barcoded target nucleic acid molecules are produced, wherein each barcoded target nucleic acid molecule comprises at least one nucleotide synthesised from the second target nucleic acid as a template.

The method may be performed using a library of multimeric barcoding reagents as defined herein and an adapter oligonucleotide as defined herein for each of the multimeric barcoding reagents. Preferably, the adapter oligonucleotides of the first multimeric barcoding reagent anneal to sub-sequences of a first target nucleic acid and first and second different target nucleic acid molecules are produced, wherein each target nucleic acid molecule comprises at least one nucleotide synthesised from the first target nucleic acid as a template; and the adapter oligonucleotides of the second multimeric barcoding reagent anneal to sub-sequences of a second target nucleic acid and first and second different target nucleic acid molecules are produced, wherein each target nucleic acid molecule comprises at least one nucleotide synthesised from the second target nucleic acid as a template.

The barcoded-adapter oligonucleotides may be isolated from the nucleic acid sample after annealing to the sub-sequences of the target nucleic acid and before the barcoded target nucleic acid molecules are produced. Optionally, the barcoded-adapter oligonucleotides are isolated by capture on a solid support through a streptavidin-biotin interaction.

The barcoded target nucleic acid molecules may be isolated from the nucleic acid sample. Optionally, the barcoded target nucleic acid molecules are isolated by capture on a solid support through a streptavidin-biotin interaction.

In the method first and second adapter oligonucleotides are annealed to a target nucleic acid in the nucleic acid sample, and then used in a primer extension reaction. Each adapter oligonucleotide is comprised of an adapter region that is complementary to, and thus able to anneal to, the 5′ adapter region of a barcode molecule. Each adapter oligonucleotide is also comprised of a target region, which may be used to anneal the barcoded oligonucleotides to target nucleic acids, and then may be used as primers for a primer-extension reaction or a polymerase chain reaction. These adapter oligonucleotides may be synthesised to include a 5′-terminal phosphate group.

The adapter oligonucleotides, each of which has been extended to include sequence from the target nucleic acid, are then contacted with a multimeric barcoding reagent which comprises a first and second barcode molecule, as well as first and second barcoded oligonucleotides, which each comprise a barcode region, as well as 5′ regions. The first and second barcode molecules each comprise a barcode region, an adapter region, and a 3′ region, and are linked together, in this embodiment by a connecting nucleic acid sequence.

After contacting the primer-extended nucleic acid sample with a multimeric barcoding reagent, the 5′ adapter regions of each adapter oligonucleotides are able to anneal to a ‘ligation junction’ adjacent to the 3′ end of each barcoded oligonucleotide. The 5′ end of the extended adapter oligonucleotides are then ligated to the 3′ end of the barcoded oligonucleotides within the multimeric barcoding reagent, creating a ligated base pair where the ligation junction was formerly located. The solution may subsequently be processed further or amplified, and used in a sequencing reaction.

This method creates barcoded target nucleic acid molecules, wherein two or more sub-sequences from the nucleic acid sample are labeled by a barcoded oligonucleotide. In this method a multimeric barcoding reagent does not need to be present for the step of annealing target regions to sub-sequences of the target nucleic acid, or the step of extending the annealed target regions using a polymerase. This feature may hold advantages in certain applications, for example wherein a large number of target sequences are of interest, and the target regions are able to hybridise more rapidly to target nucleic acids when they are not constrained molecularly by a multimeric barcoding reagent.

FIG. 5 shows a method of preparing a nucleic acid sample for sequencing using a multimeric barcoding reagent. In the method first (C1 and G1) and second (C2 and G2) adapter oligonucleotides are annealed to a target nucleic acid in the nucleic acid sample, and then used in a primer extension reaction. Each adapter oligonucleotide is comprised of an adapter region (C1 and C2) that is complementary to, and thus able to anneal to, the 5′ adapter region of a barcode molecule (F1 and F2). Each adapter oligonucleotide is also comprised of a target region (G1 and G2), which may be used to anneal the barcoded oligonucleotides to target nucleic acids, and then may be used as primers for a primer-extension reaction or a polymerase chain reaction. These adapter oligonucleotides may be synthesised to include a 5′-terminal phosphate group.

The adapter oligonucleotides, each of which has been extended to include sequence from the target nucleic acid, are then contacted with a multimeric barcoding reagent which comprises a first (D1, E1, and F1) and second (D2, E2, and F2) barcode molecule, as well as first (A1 and B1) and second (A2 and B2) barcoded oligonucleotides, which each comprise a barcode region (B1 and B2), as well as 5′ regions (A1 and A2). The first and second barcode molecules each comprise a barcode region (E1 and E2), an adapter region (F1 and F2), and a 3′ region (D1 and D2), and are linked together, in this embodiment by a connecting nucleic acid sequence(S).

After contacting the primer-extended nucleic acid sample with a multimeric barcoding reagent, the 5′ adapter regions (C1 and C2) of each adapter oligonucleotides are able to anneal to a ‘ligation junction’ adjacent to the 3′ end of each barcoded oligonucleotide (J1 and J2). The 5′ end of the extended adapter oligonucleotides are then ligated to the 3′ end of the barcoded oligonucleotides within the multimeric barcoding reagent, creating a ligated base pair (K1 and K2) where the ligation junction was formerly located. The solution may subsequently be processed further or amplified, and used in a sequencing reaction.

This method, like the methods illustrated in FIGS. 3 and 4, creates barcoded target nucleic acid molecules, wherein two or more sub-sequences from the nucleic acid sample are labeled by a barcoded oligonucleotide. In this method a multimeric barcoding reagent does not need to be present for the step of annealing target regions to sub-sequences of the target nucleic acid, or the step of extending the annealed target regions using a polymerase. This feature may hold advantages in certain applications, for example wherein a large number of target sequences are of interest, and the target regions are able to hybridise more rapidly to target nucleic acids when they are not constrained molecularly by a multimeric barcoding reagent.

### 15. METHODS OF PREPARING A NUCLEIC ACID SAMPLE FOR SEQUENCING USING MULTIMERIC BARCODING REAGENTS, ADAPTER OLIGONUCLEOTIDES AND EXTENSION PRIMERS

The invention further provides a method of preparing a nucleic acid sample for sequencing, wherein the method comprises the steps of: (a) contacting the nucleic acid sample with first and second adapter oligonucleotides as defined herein; (b) annealing the target region of the first adapter oligonucleotide to a first sub-sequence of a target nucleic acid, and annealing the target region of the second adapter oligonucleotide to a second sub-sequence of the target oligonucleotide; (c) contacting the nucleic acid sample with a library of multimeric barcode molecules as defined herein and first and second extension primers as defined herein; (d) annealing the adapter region of the first adapter oligonucleotide to the adapter region of the first barcode molecule, and annealing the adapter region of the second adapter oligonucleotide to the adapter region of the second barcode molecule; (e) extending the first extension primer using the barcode region of the first barcode molecule as a template to produce a first barcoded oligonucleotide, and extending the second extension primer using the barcode region of the second barcode molecule as a template to produce a second barcoded oligonucleotide, wherein the first barcoded oligonucleotide comprises a sequence complementary to the barcode region of the first barcode molecule and the second barcoded oligonucleotide comprises a sequence complementary to the barcode region of the second barcode molecule; and (f) ligating the 3′ end of the first barcoded oligonucleotide to the 5′ end of the first adapter oligonucleotide to produce a first barcoded-adapter oligonucleotide and ligating the 3′ end of the second barcoded oligonucleotide to the 5′ end of the second adapter oligonucleotide to produce a second barcoded-adapter oligonucleotide.

In the method the first and second barcoded-adapter oligonucleotides may be extended to produce first and second different barcoded target nucleic acid molecules each of which comprises at least one nucleotide synthesised from the target nucleic acid as a template.

Alternatively, the first and second adapter oligonucleotides may be extended to produce first and second different target nucleic acid molecules each of which comprises at least one nucleotide synthesised from the target nucleic acid as a template. In this method, step (f) produces a first barcoded target nucleic acid molecule (i.e. the first barcoded oligonucleotide ligated to the extended first adapter oligonucleotide) and a second barcoded target nucleic acid molecule (i.e. the second barcoded oligonucleotide ligated to the extended second adapter oligonucleotide).

The step of extending the adapter oligonucleotides may be performed before step (c), before step (d), before step (e) and/or before step (f), and the first and second adapter oligonucleotides may remain annealed to the first and second barcode molecules until after step (f).

The extension primers may be annealed to the multimeric barcode molecules prior to step (c). Alternatively, the nucleic acid sample may be contacted with a library of multimeric barcode molecules as defined herein and separate extension primers as defined herein. The extension primers may then be annealed to the multimeric barcode molecules in the nucleic acid sample. The extension primers may be annealed to the multimeric barcode molecules during step (d).

The methods may use a library of first and second extension primers e.g. the library may comprise first and second extension primers for each multimeric barcode molecule. Optionally, each extension primer in the library of extension primers may comprise a secondary barcode region, wherein said secondary barcode region is different to the secondary barcode regions within the other extension primers within the library. Optionally, such a library may comprise at least 2, at least 3, at least 4, at least 5, at least 10, at least 20, at least 50, at least 100, at least 500, at least 1000, at least 5,000, or at least 10,000 different extension primers.

### 16. METHODS OF PREPARING A NUCLEIC ACID SAMPLE FOR SEQUENCING USING MULTIMERIC BARCODING REAGENTS, ADAPTER OLIGONUCLEOTIDES AND TARGET PRIMERS

The invention further provides a method of preparing a nucleic acid sample for sequencing, wherein the method comprises the steps of: (a) contacting the nucleic acid sample with first and second adapter oligonucleotides, wherein each adapter oligonucleotide comprises in the 5′ to 3′ direction a target region and an adapter region, and first and second target primers; (b) annealing the target region of the first adapter oligonucleotide to a first sub-sequence of a target nucleic acid, and annealing the target region of the second adapter oligonucleotide to a second sub-sequence of the target nucleic acid; (c) annealing the first target primer to a third sub-sequence of the target nucleic acid, wherein the third sub-sequence is 3′ of the first sub-sequence, and annealing the second target primer to a fourth sub-sequence of the target nucleic acid, wherein the fourth sub-sequence is 3′ of the second sub-sequence; (d) extending the first target primer using the target nucleic acid as template until it reaches the first sub-sequence to produce a first extended target primer, and extending the second target primer using the target nucleic acid as template until it reaches the second sub-sequence to produce a second extended target primer; (e) ligating the 3′ end of the first extended target primer to the 5′ end of the first adapter oligonucleotide, and ligating the 3′ end of the second extended target primer to the 5′ end of the second adapter oligonucleotide; (f) contacting the nucleic acid sample with a library of multimeric barcode molecules as defined herein; (g) annealing the adapter region of the first adapter oligonucleotide to the adapter region of the first barcode molecule, and annealing the adapter region of the second adapter oligonucleotide to the adapter region of the second barcode molecule; and (h) extending the first adapter oligonucleotide using the barcode region of the first barcode molecule as a template to produce a first barcoded oligonucleotide, and extending the second adapter oligonucleotide using the barcode region of the second barcode molecule as a template to produce a second barcoded oligonucleotide, wherein the first barcoded oligonucleotide comprises a sequence complementary to the barcode region of the first barcode molecule and the second barcoded oligonucleotide comprises a sequence complementary to the barcode region of the second barcode molecule.

In the method, steps (b) and (c) may be performed at the same time.

In the method, steps (f)-(h) may be performed before steps (d) and (e). In this method, first and second different barcoded target nucleic acid molecules, each of which comprises at least one nucleotide synthesised from the target nucleic acid as a template, are produced by the completion of step (e).

In the method, steps (f)-(h) may be performed after steps (d) and (e). In this method, first and second different barcoded target nucleic acid molecules, each of which comprises at least one nucleotide synthesised from the target nucleic acid as a template, are produced by the completion of step (h).

In this method, the target nucleic acid may be any type of nucleic acid e.g genomic DNA or an RNA molecule such as an mRNA molecule.

FIG. 6 illustrates one way in which this method may be performed. In this method, the target nucleic acid is genomic DNA. It will be appreciated that the target nucleic acid may be another type of nucleic acid e.g. an RNA molecule such as an mRNA molecule.

### 17. METHODS OF PREPARING A NUCLEIC ACID SAMPLE FOR SEQUENCING USING MULTIMERIC BARCODING REAGENTS AND TARGET PRIMERS

The invention further provides a method of preparing a nucleic acid sample for sequencing, wherein the method comprises the steps of: (a) contacting the nucleic acid sample with first and second barcoded oligonucleotides linked together, wherein each barcoded oligonucleotide comprises in the 5′ to 3′ direction a target region and a barcode region, and first and second target primers; (b) annealing the target region of the first barcoded oligonucleotide to a first sub-sequence of a target nucleic acid, and annealing the target region of the second barcoded oligonucleotide to a second sub-sequence of the target nucleic acid; (c) annealing the first target primer to a third sub-sequence of the target nucleic acid, wherein the third sub-sequence is 3′ of the first sub-sequence, and annealing the second target primer to a fourth sub-sequence of the target nucleic acid, wherein the fourth sub-sequence is 3′ of the second sub-sequence; (d) extending the first target primer using the target nucleic acid as template until it reaches the first sub-sequence to produce a first extended target primer, and extending the second target primer using the target nucleic acid as template until it reaches the second sub-sequence to produce a second extended target primer; (e) ligating the 3′ end of the first extended target primer to the 5′ end of the first barcoded oligonucleotide to produce a first barcoded target nucleic acid molecule, and ligating the 3′ end of the second extended target primer to the 5′ end of the second barcoded oligonucleotide to produce a second barcoded target nucleic acid molecule, wherein the first and second barcoded target nucleic acid molecules are different and each comprises at least one nucleotide synthesised from the target nucleic acid as a template.

### 18. METHODS OF ASSEMBLING MULTIMERIC BARCODE MOLECULES BY ROLLING CIRCLE AMPLIFICATION

The invention further provides a method of assembling a library of multimeric barcode molecules from a library of nucleic acid barcode molecules, wherein said nucleic acid barcode molecules are amplified by one or more rolling circle amplification (RCA) processes. In this method, nucleic acid barcode molecules may each comprise, optionally in the 5′ to 3′ direction, a barcode region and an adapter region. Optionally, the nucleic acid barcode molecules may comprise a phosphorylated 5′ end capable of ligating to a 3′ end of a nucleic acid molecule.

In this method, nucleic acid barcode molecules within the library are converted into a circular form, such that the barcode region and the adapter region from a barcode molecule are comprised within a contiguous circular nucleic acid molecule. Optionally, such a step of converting nucleic acid barcode molecules into circular form may be performed by an intramolecular single-stranded ligation reaction. For example, nucleic acid barcode molecules comprising a phosphorylated 5′ end may be circularised by incubation with a single-stranded nucleic acid ligase, such as T4 RNA Ligase 1, or by incubation with a thermostable single-stranded nucleic acid ligase, such as the CircLigase thermostable single-stranded nucleic acid ligase (from Epicentre Bio). Optionally, an exonuclease step may be performed to deplete or degrade uncircularised and/or unligated molecules; optionally wherein the exonuclease step is performed by E. coli exonuclease I, or by E. coli lambda exonuclease.

Optionally, a step of converting nucleic acid barcode molecules into circular form may be performed using a circularisation primer. In this embodiment, nucleic acid barcode molecules comprise a phosphorylated 5′ end. Furthermore, in this embodiment, a circularisation primer comprising a 5′ region complementary to the 3′ region of a barcode molecule, and a 3′ region complementary to the 5′ region of a barcode molecule, is annealed to a barcode molecule, such that the 5′ end and the 3′ end of the barcode molecule are immediately adjacent to each other whilst annealed along the circularisation primer. Following the annealing step, the annealed barcode molecules are ligated with a ligase enzyme, such as T4 DNA ligase, which ligates the 3′ end of the barcode molecule to the 5′ end of the barcode molecule. Optionally, an exonuclease step may be performed to deplete or degrade uncircularised and/or unligated molecules; optionally wherein the exonuclease step is performed by E. coli exonuclease I, or by E. coli lambda exonuclease.

Following a circularisation step, circularised barcode molecules may be amplified with a rolling circle amplification step. In this process, a primer is annealed to a circularised nucleic acid strand comprising a barcode molecule, and the 3′ end of said primer is extended with a polymerase exhibiting strand displacement behaviour. For each original circularised barcode molecule, this process may form a linear (non-circular) multimeric barcode molecule comprising copies of the original circularised barcode molecule, as illustrated in FIG. 7. In one embodiment, a circularisation primer that has been annealed to a barcode molecule may serve as the primer for a rolling circle amplification step. Optionally, following circularisation, a separate amplification primer which is at least partially complementary to the circularised barcode molecule, may be annealed to the circularised barcode molecule to prime a rolling circle amplification step.

During said rolling circle amplification step, the primer may be extended by the polymerase, wherein the polymerase extends along the circularised template until it encounters the 5′ end of the amplification primer and/or circularisation primer, whereupon it continues amplification along the circularised template whilst displacing the 5′ end of the primer, and then displacing the previously amplified strand, in a process of rolling circle amplification. Following any such amplification step, a purification and/or cleanup step may be performed to isolate products of such rolling circle amplification. Optionally, a purification and/or cleanup step may comprise a size-selection process, such as a gel-based size selection process, or a solid-phase reversible immobilisation size-selection process, such as a magnetic bead-based solid-phase reversible immobilisation size-selection process. Optionally, amplification products at least 100 nucleotides in length, at least 500 nucleotides in length, at least 1000 nucleotides in length, at least 2000 nucleotides in length, at least 5000 nucleotides in length, at least 10,000 nucleotides in length, at least 20,000 nucleotides in length, at least 50,000 nucleotides in length, or at least 100,000 nucleotides in length may be purified. Optionally, before and/or during any rolling circle amplification step, a single-stranded DNA binding protein (such as T4 Gene 32 Protein) may be included in a reaction mixture, such as to prevent the formation of secondary structures by circularised templates and/or amplification products. During or after any such rolling circle amplification step, said single-stranded DNA binding protein may be removed and/or inactivated, such as by a heat-inactivation step.

Optionally, such a process of rolling circle amplification may be performed by phi29 DNA polymerase. Optionally, such a process of rolling circle amplification may be performed by a Bst or Bsm DNA polymerase. Optionally, such a process of rolling circle amplification may be performed such that at least one full copy of the circularised template is produced by the polymerase. Optionally, such a process of rolling circle amplification may be performed such that at least 2, at least 3, at least 5, at least 10, at least 50, at least 100, at least 200, at least 500, at least 1000, at least 2000, at least 5000, or at least 10,000 full copies of the circularised template are produced by the polymerase.

An example of this method is provided in FIG. 7. In the figure, a barcode molecule comprising an adapter region and a barcode region is circularised (e.g. using a single-stranded ligation reaction). A primer is then annealed to the resulting circularised product, and said primer is then extended using a strand-displacing polymerase (such as phi29 DNA polymerase). Whilst synthesising the extension product, the polymerase then processes one circumference around the circularised product, and then displaces the original primer in a strand-displacement reaction. The rolling-circle amplification process may then proceed to create a long contiguous nucleic acid molecule comprising many tandem copies of the circularised sequence—i.e. many tandem copies of a barcode and adapter sequence (and/or sequences complementary to a barcode and adapter sequence) of a barcode molecule.

Multimeric barcode molecules may also be amplified by rolling circle amplification.

### 19. METHODS OF SYNTHESISING A MULTIMERIC BARCODING REAGENT

The invention further provides a method of synthesising a multimeric barcoding reagent for labelling a target nucleic acid comprising: (a) contacting first and second barcode molecules with first and second extension primers, wherein each of the barcode molecules comprises a single-stranded nucleic acid comprising in the 5′ to 3′ direction an adapter region, a barcode region and a priming region; (b) annealing the first extension primer to the priming region of the first barcode molecule and annealing the second extension primer to the priming region of the second barcode molecule; and (c) synthesising a first barcoded extension product by extending the first extension primer and synthesising a second barcoded extension product by extending the second extension primer, wherein the first barcoded extension product comprises a sequence complementary to the barcode region of the first barcode molecule and the second barcoded extension product comprises a sequence complementary to the barcode region of the second barcode molecule, and wherein the first barcoded extension product does not comprise a sequence complementary to the adapter region of the first barcode molecule and the second barcoded extension product does not comprise a sequence complementary to the adapter region of the second barcode molecule; and wherein the first and second barcode molecules are linked together.

The method may further comprise the following steps before the step of synthesising the first and second barcoded extension products: (a) contacting first and second barcode molecules with first and second blocking primers; and (b) annealing the first blocking primer to the adapter region of the first barcode molecule and annealing the second blocking primer to the adapter region of the second barcode molecule; and wherein the method further comprises the step of dissociating the blocking primers from the barcode molecules after the step of synthesising the barcoded extension products.

In the method, the extension step, or a second extension step performed after the synthesis of an extension product, may be performed, in which one or more of the four canonical deoxyribonucleotides is excluded from the extension reaction, such that the second extension step terminates at a position before the adapter region sequence, wherein the position comprises a nucleotide complementary to the excluded deoxyribonucleotide. This extension step may be performed with a polymerase lacking 3′ to 5′ exonuclease activity.

The barcode molecules may be provided by a single-stranded multimeric barcode molecule as defined herein.

The barcode molecules may be synthesised by any of the methods defined herein. The barcode regions may uniquely identify each of the barcode molecules. The barcode molecules may be linked on a nucleic acid molecule. The barcode molecules may be linked together in a ligation reaction. The barcode molecules may be linked together by a further step comprising attaching the barcode molecules to a solid support.

The first and second barcode molecules may be assembled as a double-stranded multimeric barcode molecule by any of the methods defined herein prior to step (a) defined above (i.e. contacting first and second barcode molecules with first and second extension primers). The double-stranded multimeric barcode molecule may be dissociated to produce single-stranded multimeric barcode molecules for use in step (a) defined above (i.e. contacting first and second barcode molecules with first and second extension primers).

The method may further comprise the steps of: (a) annealing an adapter region of a first adapter oligonucleotide to the adapter region of the first barcode molecule and annealing an adapter region of a second adapter oligonucleotide to the adapter region of the second barcode molecule, wherein the first adapter oligonucleotide further comprises a target region capable of annealing to a first sub-sequence of the target nucleic acid and the second adapter oligonucleotide further comprises a target region capable of annealing to a second sub-sequence of the target nucleic acid; and (b) ligating the 3′ end of the first barcoded extension product to the 5′ end of the first adapter oligonucleotide to produce a first barcoded oligonucleotide and ligating the 3′ end of the second barcoded extension product to the 5′ end of the second adapter oligonucleotide to produce a second barcoded oligonucleotide. Optionally, the annealing step (a) may be performed before the step of synthesising the first and second barcoded extension products and wherein the step of synthesising the first and second barcoded extension products is conducted in the presence of a ligase enzyme that performs the ligation step (b). The ligase may be a thermostable ligase. The extension and ligation reaction may proceed at over 37 degrees Celsius, over 45 degrees Celsius, or over 50 degrees Celsius.

The target regions may comprise different sequences. Each target region may comprise a sequence capable of annealing to only a single sub-sequence of a target nucleic acid within a sample of nucleic acids. Each target region may comprise one or more random, or one or more degenerate, sequences to enable the target region to anneal to more than one sub-sequence of a target nucleic acid. Each target region may comprise at least 5, at least 10, at least 15, at least 20, at least 25, at least 50 or at least 100 nucleotides. Preferably, each target region comprises at least 5 nucleotides. Each target region may comprise 5 to 100 nucleotides, 5 to 10 nucleotides, 10 to 20 nucleotides, 20 to 30 nucleotides, 30 to 50 nucleotides, 50 to 100 nucleotides, 10 to 90 nucleotides, 20 to 80 nucleotides, 30 to 70 nucleotides or 50 to 60 nucleotides. Preferably, each target region comprises 30 to 70 nucleotides. Preferably each target region comprises deoxyribonucleotides, optionally all of the nucleotides in a target region are deoxyribonucleotides. One or more of the deoxyribonucleotides may be a modified deoxyribonucleotide (e.g. a deoxyribonucleotide modified with a biotin moiety or a deoxyuracil nucleotide). Each target region may comprise one or more universal bases (e.g. inosine), one or modified nucleotides and/or one or more nucleotide analogues.

The adapter region of each adapter oligonucleotide may comprise a constant region. Optionally, all adapter regions of adapter oligonucleotides that anneal to a single multimeric barcoding reagent are substantially identical. The adapter region may comprise at least 4, at least 5, at least 6, at least 8, at least 10, at least 15, at least 20, at least 25, at least 50, at least 100, or at least 250 nucleotides. Preferably, the adapter region comprises at least 4 nucleotides. Preferably each adapter region comprises deoxyribonucleotides, optionally all of the nucleotides in an adapter region are deoxyribonucleotides. One or more of the deoxyribonucleotides may be a modified deoxyribonucleotide (e.g. a deoxyribonucleotide modified with a biotin moiety or a deoxyuracil nucleotide). Each adapter region may comprise one or more universal bases (e.g. inosine), one or modified nucleotides and/or one or more nucleotide analogues.

For any of the methods involving adapter oligonucleotides, the 3′ end of the adapter oligonucleotide may include a reversible terminator moiety or a reversible terminator nucleotide (for example, a 3′-O-blocked nucleotide), for example at the 3′ terminal nucleotide of the target region. When used in an extension and/or extension and ligation reaction, the 3′ ends of these adapter oligonucleotides may be prevented from priming any extension events. This may minimize mis-priming or other spurious extension events during the production of barcoded oligonucleotides. Prior to using the assembled multimeric barcoding reagents, the terminator moiety of the reversible terminator may be removed by chemical or other means, thus allowing the target region to be extended along a target nucleic acid template to which it is annealed.

Similarly, for any of the methods involving adapter oligonucleotides, one or more blocking oligonucleotides complementary to one or more sequences within the target region(s) may be employed during extension and/or extension and ligation reactions. The blocking oligonucleotides may comprise a terminator and/or other moiety on their 3′ and/or 5′ ends such that they are not able to be extended by polymerases. The blocking oligonucleotides may be designed such that they anneal to sequences fully or partially complementary to one or more target regions, and are annealed to said target regions prior to an extension and/or extension and ligation reaction. The use of blocking primers may prevent target regions from annealing to, and potentially mis-priming along, sequences within the solution for which such annealing is not desired (for example, sequence features within barcode molecules themselves). The blocking oligonucleotides may be designed to achieve particular annealing and/or melting temperatures. Prior to using the assembled multimeric barcoding reagents, the blocking oligonucleotide(s) may then be removed by, for example, heat-denaturation and then size-selective cleanup, or other means. The removal of the blocking oligonucleotide(s) may allow the target region to be extended along a target nucleic acid template to which it is annealed.

The method may comprise synthesising a multimeric barcoding reagent comprising at least 5, at least 10, at least 20, at least 25, at least 50, at least 75 or at least 100 barcode molecules, and wherein: (a) each barcode molecule is as defined herein; and (b) a barcoded extension product is synthesised from each barcode molecule according to any method defined herein; and, optionally, (c) an adapter oligonucleotide is ligated to each of the barcoded extension products to produce barcoded oligonucleotides according to any of the methods defined herein.

The invention further provides a method of synthesising a library of multimeric barcoding reagents, wherein the method comprises repeating the steps of any of the methods defined herein to synthesise two or more multimeric barcoding reagents. Optionally, the method comprises synthesising a library of at least 5, at least 10, at least 20, at least 25, at least 50, at least 75, at least 100, at least 250, at least 500, at least 10³, at least 10⁴, at least 10⁵, at least 10⁶, at least 10⁷, at least 10⁸, at least 10⁹ or at least 10¹⁰ multimeric barcoding reagents as defined herein. Preferably, the library comprises at least 5 multimeric barcoding reagents as defined herein. Preferably, the barcode regions of each of the multimeric barcoding reagents may be different to the barcode regions of the other multimeric barcoding reagents.

FIG. 8 illustrates a method of synthesizing a multimeric barcoding reagent for labeling a target nucleic acid. In this method, first (D1, E1, and F1) and second (D2, E2, and F2) barcode molecules, which each include a nucleic acid sequence comprising a barcode region (E1 and E2), and which are linked by a connecting nucleic acid sequence(S), are denatured into single-stranded form. To these single-stranded barcode molecules, a first and second extension primer (A1 and A2) is annealed to the 3′ region of the first and second barcode molecules (D1 and D2), and a first and second blocking primer (R1 and R2) is annealed to the 5′ adapter region (F1 and F2) of the first and second barcode molecules. These blocking primers (R1 and R2) may be modified on the 3′ end such that they cannot serve as a priming site for a polymerase.

A polymerase is then used to perform a primer extension reaction, in which the extension primers are extended to make a copy (B1 and B2) of the barcode region of the barcode molecules (E1 and E2). This primer extension reaction is performed such that the extension product terminates immediately adjacent to the blocking primer sequence, for example through use of a polymerase which lacks strand displacement or 5′-3′ exonuclease activity. The blocking primers (R1 and R2) are then removed, for example through high-temperature denaturation.

This method thus creates a multimeric barcoding reagent containing a first and second ligation junction (J1 and J2) adjacent to a single-stranded adapter region (F1 and F2). This multimeric barcoding reagent may be used in the method illustrated in FIG. 5.

The method may further comprise the step of ligating the 3′ end of the first and second barcoded oligonucleotides created by the primer-extension step (the 3′ end of B1 and B2) to first (C1 and G1) and second (C2 and G2) adapter oligonucleotides, wherein each adapter oligonucleotide comprises an adapter region (C1 and C2) which is complementary to, and thus able to anneal to, the adapter region of a barcode molecule (F1 and F2). The adapter oligonucleotides may be synthesised to include a 5′-terminal phosphate group.

Each adapter oligonucleotide may also comprise a target region (G1 and G2), which may be used to anneal the barcoded oligonucleotides to target nucleic acids, and may separately or subsequently be used as primers for a primer-extension reaction or a polymerase chain reaction. The step of ligating the first and second barcoded oligonucleotides to the adapter oligonucleotides produces a multimeric barcoding reagent as illustrated in FIG. 1 that may be used in the methods illustrated in FIG. 3 and/or FIG. 4.

FIG. 9 shows a method of synthesizing multimeric barcoding reagents (as illustrated in FIG. 1) for labeling a target nucleic acid. In this method, first (D1, E1, and F1) and second (D2, E2, and F2) barcode molecules, which each include a nucleic acid sequence comprising a barcode region (E1 and E2), and which are linked by a connecting nucleic acid sequence(S), are denatured into single-stranded form. To these single-stranded barcode molecules, a first and second extension primer (A1 and A2) is annealed to the 3′ region of the first and second barcode molecules (D1 and D2), and the adapter regions (C1 and C2) of first (C1 and G1) and second (C2 and G2) adapter oligonucleotides are annealed to the 5′ adapter regions (F1 and F2) of the first and second barcode molecules. These adapter oligonucleotides may be synthesised to include a 5′-terminal phosphate group.

A polymerase is then used to perform a primer extension reaction, in which the extension primers are extended to make a copy (B1 and B2) of the barcode region of the barcode molecules (E1 and E2). This primer extension reaction is performed such that the extension product terminates immediately adjacent to the adapter region (C1 and C2) sequence, for example through use of a polymerase which lacks strand displacement or 5′-3′ exonuclease activity.

A ligase enzyme is then used to ligate the 5′ end of the adapter oligonucleotides to the adjacent 3′ end of the corresponding extension product. In an alternative embodiment, a ligase enzyme may be included with the polymerase enzyme in one reaction which simultaneously effects both primer-extension and ligation of the resulting product to the adapter oligonucleotide. Through this method, the resulting barcoded oligonucleotides may subsequently be used as primers for a primer-extension reaction or a polymerase chain reaction, for example as in the method shown in FIG. 3 and/or FIG. 4.

The invention further provides a method of synthesising a multimeric barcoding reagent comprising appending one or more (donor) multimeric barcoding reagents to a support. Multimeric hybridization molecules (e.g. multimeric barcode molecules) may be appended to a support. Additionally or alternatively, barcoded oligonucleotides, which may have been synthesised from a multimeric barcode molecule, may be appended to a support. The support may be any support described herein e.g. a macromolecule, solid support or semi-solid support.

The support may be selected based on the desired structural and/or functional properties of the multimeric barcoding reagent. For example: barcoded oligonucleotides may be appended to magnetic beads. This may allow a laboratory scientist to easily manipulate the barcoded oligonucleotides, for example to perform washing steps, or purification steps. Furthermore, the functional properties of the bead may enable a scientist to isolate or purify nucleic acids from a nucleic acid sample that may be hybridised to and/or barcoded with the barcoded oligonucleotides. Furthermore, appending barcoded oligonucleotides to a support may change the overall structural nature of the barcoded oligonucleotides. For example, appending barcoded oligonucleotides to a streptavidin tetramer may change the three-dimensional structure of the barcoded oligonucleotides such that cross-hybridization between the target regions of different barcoded oligonucleotides is reduced, thereby reducing the amount of potential mis-priming between barcoded oligonucleotides, and/or enhancing the accessibility of the target regions to potential target nucleic acids within a sample.

Qualitative and quantitative assays may be used to assess the production of functional multimeric barcoding reagents. For example, the correct linkage of an oligonucleotide (e.g. a multimeric hybridization molecule) to a support may be tested using a complementary oligonucleotide. Comparing either absorbance or fluorescence before and after annealing may be used to provide an estimate of the degree of linkage to the support. Other techniques may be used to evaluate the amount of barcoded oligonucleotides and/or cell-binding oligonucleotides forming part of a multimeric barcoding reagent. For example a qPCR extension assay may be used e.g. where the oligonucleotide of interest is either directly quantified or it is used in a competition assay with a different oligonucleotide that is in turn directly quantified.

### 20. METHODS OF SEQUENCING AND/OR PROCESSING SEQUENCING DATA

The invention further provides a method of sequencing a sample, wherein the sample has been prepared by any one of the methods of preparing a nucleic acid sample for sequencing as defined herein. The method of sequencing the sample comprises the steps of: isolating the barcoded target nucleic acid molecules, and producing a sequence read from each barcoded target nucleic acid molecule that comprises the barcode region, the target region and at least one additional nucleotide from the target nucleic acid. Each sequence read may comprise at least 5, at least 10, at least 25, at least 50, at least 100, at least 250, at least 500, at least 1000, at least 2000, at least 5000, or at least 10,000 nucleotides from the target nucleic acid. Preferably, each sequence read comprises at least 5 nucleotides from the target nucleic acid.

The methods may produce a sequence read from one or more barcoded target nucleic acid molecule produced from at least at least 10, at least 100, or at least 10³, at least 10⁴, at least 10⁵, at least 10⁶, at least 10⁷, at least 10⁸ or at least 10⁹ different target nucleic acids.

Sequencing may be performed by any method known in the art. For example, by chain-termination or Sanger sequencing. Preferably, sequencing is performed by a next-generation sequencing method such as sequencing by synthesis, sequencing by synthesis using reversible terminators (e.g. Illumina sequencing), pyrosequencing (e.g. 454 sequencing), sequencing by ligation (e.g. SOLID sequencing), single-molecule sequencing (e.g. Single Molecule, Real-Time (SMRT) sequencing, Pacific Biosciences), or by nanopore sequencing (e.g. on the Minion or Promethion platforms, Oxford Nanopore Technologies).

The invention further provides a method for processing sequencing data obtained by any of the methods defined herein. The method for processing sequence data comprises the steps of: (a) identifying for each sequence read the sequence of the barcode region and the sequence from the target nucleic acid; and (b) using the information from step (a) to determine a group of sequences from the target nucleic acid that were labelled with barcode regions from the same multimeric barcoding reagent.

The method may further comprise the step of determining a sequence of a target nucleic acid by analysing the group of sequences to identify contiguous sequences, wherein the sequence of the target nucleic acid comprises nucleotides from at least two sequence reads.

The target nucleic acid may be an intact nucleic acid molecule, co-localised fragments of a nucleic acid molecule, or nucleic acid molecules from a single cell. Preferably, the target nucleic acid is a single intact nucleic acid molecule, two or more co-localised fragments of a single nucleic acid molecule, or two or more nucleic acid molecules from a single cell.

The invention further provides an algorithm for processing (or analysing) sequencing data obtained by any of the methods defined herein. The algorithm may be configured to perform any of the methods for processing sequencing data defined herein. The algorithm may be used to detect the sequence of a barcode region within each sequence read, and also to detect the sequence within a sequence read that is derived from a target nucleic acid, and to separate these into two associated data sets.

The invention further provides a method of generating a synthetic long read from a target nucleic acid comprising the steps of: (a) preparing a nucleic acid sample for sequencing according to any of the methods defined herein; (b) sequencing the sample, optionally wherein the sample is sequenced by any of the methods defined herein; and (c) processing the sequence data obtained by step (b), optionally wherein the sequence data is processed according to any of the methods defined herein; wherein step (c) generates a synthetic long read comprising at least one nucleotide from each of the at least two sequence reads.

The method may enable the phasing of a target sequence of a target nucleic acid molecule i.e. it may enable the determination of which copy of a chromosome (i.e. paternal or maternal) the sequence is located. The target sequence may comprise a specific target mutation, translocation, deletion or amplification and the method may be used to assign the mutation, translocation, deletion or amplification to a specific chromosome. The phasing two or more target sequences may also enable the detection of aneuploidy.

The synthetic long read may comprise at least 50, at least 100, at least 250, at least 500, at least 750, at least 1000, at least 2000, at least 10⁴, at least 10⁵, at least 10⁶, at least 10⁷ or at least 10⁸ nucleotides. Preferably, the synthetic long read comprises at least 50 nucleotides.

The invention further provides a method of sequencing two or more co-localised target nucleic acids comprising the steps of: (a) preparing a nucleic acid sample for sequencing according to any of the methods defined herein; (b) sequencing the sample, optionally wherein the sample is sequenced by any of the methods defined herein; and (c) processing the sequence data obtained by step (b), optionally wherein the sequence data is processed according to any of the methods defined herein; wherein step (c) identifies at least two sequence reads comprising nucleotides from at least two target nucleic acids co-localised in the sample.

The invention further provides a method of sequencing target nucleic acids from an individual cell comprising the steps of: (a) preparing a nucleic acid sample for sequencing according any of the methods defined herein, wherein the multimeric barcoding reagent(s), or multimeric barcode molecule(s), and/or adapter oligonucleotides are introduced into the cell; (b) sequencing the sample, optionally wherein the sample is sequenced by any of the methods defined herein; and (c) processing the sequence data obtained by step (b), optionally wherein the sequence data is processed according to any of the methods defined herein; wherein step (c) identifies at least two sequence reads comprising nucleotides from at least two target nucleic acids from the cell.

The multimeric barcoding reagent(s) and/or adapter oligonucleotides may be introduced into the cell by chemical complexation with a lipid transfection reagent and then transfection into the cell.

The multimeric barcoding reagent(s) and/or adapter oligonucleotides may be introduced into the cell through the steps of: (a) permeabilising the cell membrane by contacting it with a chemical surfactant; and then (b) contacting the cell with the multimeric barcoding reagent(s) and/or adapter oligonucleotides. The chemical surfactant may be a non-ionic surfactant. The chemical surfactant may be Triton X-100 (C₁₄H₂₂O(C₂H₄O)ₙ (n=9-10)). The chemical surfactant may be in solution at a concentration of less than 200 micromolar, or less than 500 micromolar, or less than 1 milimolar.

In the method, following the step of introducing the multimeric barcoding reagent(s) and/or adapter oligonucleotides into the cell, the cell may be incubated for a period of time to allow the target regions of the multimeric barcoding reagent(s) or adapter oligonucleotide(s) to anneal to sub-sequences of the target nucleic acids within the cell. The incubation period may be at least 1 minute, or at least 5 minutes, or at least 15 minutes, or at least 30 minutes, or at least 60 minutes. Preferably, the incubation period is at least 1 minute. The incubation may take place within a solution containing a nucleic acid denaturant e.g. dimethyl sulfoxide (DMSO) or betaine. The incubation may take place at a temperature of at least 20 degrees Celsius, at least 37 degrees Celsius, at least 45 degrees Celsius, or at least 50 degrees Celsius. Preferably, the incubation takes place at a temperature of at least 20 degrees Celsius.

In methods involving the use of multimeric barcoding reagents, the incubation step may substantially dissociate the barcoded oligonucleotides from the barcode molecules (or multimeric barcode molecule). This may enable the barcoded oligonucleotides to diffuse more readily throughout the cell improving the efficiency with which the target regions of the barcoded oligonucleotides are able to anneal to sub-sequences of the target nucleic acids.

In the method, following introduction of the multimeric barcoding reagent(s) and/or adapter oligonucleotides into the cell, and optionally following the incubation step, the cell may be contacted by a solution of oligonucleotides complementary to the target regions of the multimeric barcoding reagents.

In the method, following introduction of the multimeric barcoding reagent(s) and/or adapter oligonucleotides into the cell, and optionally following the incubation step, the cell may be isolated from a reaction mixture e.g. by centrifugation.

In the method, following introduction of the multimeric barcoding reagent(s) and/or adapter oligonucleotides into the cell, and optionally following the incubation step, the barcoded oligonucleotides and/or barcoded target nucleic acid molecules and/or multimeric barcoding reagent(s) may be isolated from the cell.

The multimeric barcoding reagents, barcoded oligonucleotides and/or adapter oligonucleotides may comprise one or more biotin moieties.

In the method, following introduction of the multimeric barcoding reagent(s) and/or adapter oligonucleotides into the cell, and optionally following the incubation step, the barcoded oligonucleotides and/or barcoded target nucleic acid molecules and/or multimeric barcoding reagent(s) may be isolated by a process of: (a) optionally dissolving the cell membranes e.g. using a chemical surfactant or by incubation at high temperature; (b) contacting the resulting mixture with a solid support, optionally wherein the solid support comprises streptavidin moieties; and (c) capturing the barcoded oligonucleotides and/or barcoded target nucleic acid molecules and/or multimeric barcoding reagent(s) on the solid support, optionally through streptavidin-biotin interaction. The solid support may be one or more magnetic beads, optionally wherein the one or more magnetic beads comprise streptavidin molecules on their surface. The magnetic bead(s) may be isolated from a reaction mixture with a magnet.

The target nucleic acids may be DNA molecules (e.g. genomic DNA molecules) or RNA molecules (e.g. mRNA molecules).

Preferably, each barcoded target nucleic acid molecule is produced after isolation of the barcoded oligonucleotide annealed to a target mRNA molecule by extending the barcoded oligonucleotide using a reverse transcriptase and the target mRNA molecule as the template.

The mRNA molecules may be mRNA molecules corresponding to alpha and/or beta chains of a T-cell receptor sequence, optionally wherein the sequences of alpha and beta chains paired within an individual cell are determined.

The mRNA molecules may be mRNA molecules corresponding to light and/or heavy chains of an immunoglobulin sequence, optionally wherein the sequences of light and heavy chains paired within an individual cell are determined.

The method may be used to sequence target nucleic acids in at least 10, at least 100, or at least 10³, at least 10⁴, at least 10⁵, at least 10⁶, at least 10⁷, at least 10⁸ or at least 10⁹ cells. Preferably, the method may be used to sequence target nucleic acids in at least 10 cells. Preferably the cells are T-cells and/or B-cells.

Any method of analysing barcoded nucleic acid molecules by sequencing (e.g. to generate synthetic long reads, or to analyse nucleic acid sequences from single cells) may comprise a redundant sequencing reaction, wherein target nucleic acid molecules that have been barcoded in a barcoding reaction are sequenced two or more times within a sequencing reaction. Optionally, each such barcoded molecule from a sample may be sequenced, on average, at least twice, at least 3 times, at least 5 times, at least 10 times, at least 20 times, at least 50 times, or at least 100 times.

In any method of analysing barcoded nucleic acid molecules by sequencing (e.g. to generate synthetic long reads, or to analyse nucleic acid sequences from single cells), an error correction process may be employed. This process may comprise the steps of: (i) determining two or more sequence reads from a sequencing dataset comprising the same barcode sequence, and (ii) aligning the sequences from said two or more sequence reads to each other. Optionally, this error correction process may further comprise a step of (iii) determining a majority and/or most common and/or most likely nucleotide at each position within the sequence read and/or at each position within the sequence of the target nucleic acid molecule. This step may optionally comprise establishing a consensus sequence of each target nucleic acid sequence by any process of error correction, error removal, error detection, error counting, or statistical error removal. This step may further comprise the step of collapsing multiple sequence reads comprising the same barcode sequence into a representation comprising a single, error-corrected read. Optionally, any step of determining two or more sequence reads from a sequencing dataset comprising the same barcode sequence, may comprise determining sequence reads comprising barcode sequences with at least a certain extent of identical nucleotides and/or sequence similarity, for example at least 70%, at least 80%, at least 90%, or at least 95% sequence similarity (for example, allowing for mismatches and/or insertions or deletions at any point between to barcode sequences).

In any method of analysing barcoded nucleic acid molecules by sequencing (e.g. to generate synthetic long reads, or to analyse nucleic acid sequences from single cells), an alternative error correction process may be employed, comprising the steps of: (i) determining two or more sequence reads from a sequencing dataset that comprise the same target nucleic acid sequence, wherein said two or more sequence reads further comprise two or more different barcode sequences, wherein the barcode sequences are from the same multimeric barcode molecule and/or multimeric barcoding reagent, and (ii) aligning the sequences from said two or more sequence reads to each other. Optionally, this error correction process may further comprise a step of (iii) determining a majority and/or most common and/or most likely nucleotide at each position within the sequence of the target nucleic acid molecule. This step may optionally comprise establishing a consensus sequence of the target nucleic acid molecule by any process of error correction, error removal, error detection, error counting, or statistical error removal. This step may further comprise the step of collapsing multiple sequence reads comprising the same target nucleic acid molecule into a representation comprising a single, error-corrected read. The target nucleic acid molecule may comprise, for example, a genomic DNA sequence; alternatively, the target nucleic acid molecule may comprise all or part of a messenger RNA sequence such as an expressed gene or an expressed adaptive immune receptor chain. Optionally, any step of comparing two barcode sequences, and/or comparing a sequenced barcode sequence and a reference barcode sequence, may comprise determining sequences comprising at least a certain extent of identical nucleotides and/or sequence similarity, for example at least 70%, at least 80%, at least 90%, or at least 95% sequence similarity (for example, allowing for mismatches and/or insertions or deletions at any point between to barcode sequences).

In any method of analysing barcoded nucleic acid molecules by sequencing, the number of barcode sequences appended to specific nucleic acid targets by any given multimeric barcoding reagent, and/or across a group of two or more different multimeric barcoding reagents, may be quantitated. For example, the number of different barcode sequences from a multimeric barcoding reagent appended to a particular messenger RNA transcript (or any other specific nucleic acid targets) from a single cell may be determined. Any type of specific nucleic acid target may be quantitated, such as any transcript, any genomic DNA sequence, any synthetic barcode sequence, any adaptive immune receptor chain and/or immune receptor sequence, or any specific mutation sequence. Any such process of quantitation may be repeated for any number of specific nucleic acid targets and/or groups thereof.

### 21. USES OF A MULTIMERIC BARCODING REAGENT, LIBRARY OR KIT

The invention further provides the use of a multimeric barcoding reagent as defined herein, a library of multimeric barcoding reagents as defined herein, or a kit as defined herein, to produce two or more sequence reads from a target nucleic acid, wherein two or more sequence reads can be identified as derived from the same target nucleic acid and combined to produce a synthetic long read.

The invention further provides the use of a multimeric barcoding reagent as defined herein, a library of multimeric barcoding reagents as defined herein, or a kit as defined herein, to label a formalin-fixed paraffin-embedded (FFPE) nucleic acid sample, wherein the multimeric barcoding reagent or the components of the kit is/are introduced into the sample and used to label a set of two or more co-localised target nucleic acids for sequencing.

The multimeric barcoding reagents for use in labelling a FFPE nucleic acid sample may be less than 10 kb, less than 5 kb, less than 2 kb, less than 1 kb in length or less than 500 bp. Preferably, the multimeric barcoding reagents are less than 1 kb in length.

The invention further provides the use of a multimeric barcoding reagent as defined herein, a library of multimeric barcoding reagents as defined herein, or a kit as defined herein, to label target nucleic acids in an individual cell, wherein the multimeric barcoding reagent or the components of the kit is/are introduced into a cell and used to label a set of two or more target nucleic acids in the cell for sequencing.

The invention further provides the use of a multimeric barcoding reagent as defined herein, a library of multimeric barcoding reagents as defined herein, or a kit as defined herein, to label target nucleic acids in a sample of human plasma or serum, wherein the multimeric barcoding reagent or the components of the kit is/are used to label a set of two or more target nucleic acids in the plasma or serum for sequencing.

### 22. FURTHER METHODS OF THE INVENTION BASED ON THE ANALYSIS OF MICROPARTICLES

The methods of the invention may analyse a sample comprising a microparticle. For example, certain embodiments of the present invention may comprise reagents and/or methods for preparing nucleic acid samples containing one or more microparticles for sequencing. For example, barcode sequences may be appended from a single multimeric barcoding reagent to at least two target molecules of a microparticle (e.g. to at least two sub-sequences of a target nucleic acid of a microparticle, such as to at least two mRNA molecules of a microparticle) to produce a set of barcoded target nucleic acid molecules. Such molecules may be sequenced to produce sets of sequence reads, each set of sequence reads corresponding to nucleic acid molecules of a single microparticle (i.e. single-microparticle sequencing). In addition, the methods may be performed on many cells in parallel enabling high throughput single-cell sequencing.

Any reagents and/or methods described in the present disclosure (e.g. as relate to analysing a sample comprising a microparticle or a sample derived from a microparticle) may be employed to analyse a sample comprising a miroparticle. For example, any one or more multimeric hybridization molecules (e.g. multimeric barcode molecules) and/or any one or more multimeric barcoding reagents described herein (for example, any library or libraries of two or more multimeric hybridization molecules (e.g. multimeric barcode molecules), and/or any library or libraries of two or more multimeric barcoding reagents) may be employed by any method(s) (such as any method(s) described herein) to analyse a sample comprising a microparticle.

In certain embodiments, multimeric barcoding reagent(s) described herein may be employed by a method to produce a set of at least two (informatically) linked signals for a cell, wherein the method comprises appending each of at least two target molecules of a microparticle (for example, at least two sub-sequences of a target nucleic acid of the microparticle, such as at least two mRNA molecules of a microparticle) to a barcode sequence (such as to a barcode sequence comprised in a barcoded oligonucleotide), wherein said barcode sequences are comprised within said multimeric barcoding reagent, to produce a set of linked signals of said microparticle (such as to produce a set of barcoded target nucleic acid molecules of said microparticle); similarly, any library or libraries of two or more multimeric barcoding reagents may be employed similarly to analyse a sample comprising two or more microparticles, wherein barcode sequences from a first multimeric barcoding reagent in the library are appended to target molecules of a first microparticle in the sample, and barcode sequences from a second multimeric barcoding reagent in the library are appended to target molecules of a second microparticle in the sample.

Any reagents and/or methods described in the present disclosure (e.g. as relate to analysing a sample comprising a cell or cells) may be employed to analyse a sample comprising a microparticle or microparticles. In such embodiments, the term “cell-binding” as applied in the context of reagents and/or methods relating to the analysis of a cell or cells, is to be understood as a reference to “microparticle-binding”. For example, in such embodiments, the term “cell-binding moiety” is to be understood as “microparticle-binding moiety”.

The invention provides a multimeric barcoding reagent for labelling a target nucleic acid for sequencing, wherein the multimeric barcoding reagent comprises:
a. a support;
b. at least two multimeric hybridization molecules, wherein each multimeric hybridization molecule is independently linked to the support and wherein each multimeric hybridization molecule comprises at least two hybridization molecules linked together, wherein each of the hybridization molecules comprises a nucleic acid sequence comprising a hybridization region;
c. at least two barcoded oligonucleotides annealed to each of the multimeric hybridization molecules, wherein each barcoded oligonucleotide is annealed to one of the hybridization regions and wherein each barcoded oligonucleotide comprises a barcode region; and
d. a microparticle-binding moiety linked to each multimeric hybridization molecule.

The first end of each linear nucleic acid molecule may be linked to the support and the second end is linked to a microparticle-binding moiety.

Each microparticle-binding moiety may be linked to one of the multimeric hybridization molecules by a microparticle-binding oligonucleotide.

Each microparticle-binding oligonucleotide may be annealed to one of the multimeric hybridization molecules.

The invention provides a method of preparing a nucleic acid sample for sequencing, wherein the sample comprises at least 2 microparticles, and wherein the method comprises in order the steps of:
(a) contacting the sample with a library comprising at least two multimeric barcoding reagents, wherein the barcode regions of the barcoded oligonucleotides of a first multimeric barcoding reagent of the library are different to the barcode regions of the barcoded oligonucleotides of a second multimeric barcoding reagent of the library, wherein the microparticle-binding moiety of the first multimeric barcoding reagent binds to the membrane of a first microparticle prior to step (b), and wherein the microparticle-binding moiety of the second multimeric barcoding reagent binds to the membrane of a second microparticle prior to step (b);
(b) lysing the microparticles or permeabilizing the membranes of the microparticles; and
(c) (separately) annealing or ligating each of the barcoded oligonucleotides of the first multimeric barcoding reagent to at least four sub-sequences of a target nucleic acid of the first microparticle to produce at least four barcoded target nucleic acid molecules, and (separately) annealing or ligating each of the barcoded oligonucleotides of the second multimeric barcoding reagent to a least four sub-sequences of a target nucleic acid of the second microparticle to produce at least four barcoded target nucleic acid molecules, optionally wherein the microparticles are comprised within a single contiguous aqueous volume during steps (a), (b) and (c).

In the method, step (c) may comprise:
(i) annealing each of the barcoded oligonucleotides of the first multimeric barcoding reagent to at least four sub-sequences of a target nucleic acid of the first microparticle, and annealing each of the barcoded oligonucleotides of the second multimeric barcoding reagent to at least four sub-sequences of a target nucleic acid of the second microparticle; and
(ii) extending each of the barcoded oligonucleotides of the first multimeric barcoding reagent to produce at least four different barcoded target nucleic acid molecules and extending each of the barcoded oligonucleotides of the second multimeric barcoding reagent to produce at least four different barcoded target nucleic acid molecules, wherein each of the barcoded target nucleic acid molecules comprises at least one nucleotide synthesised from the target nucleic acid as a template.

In the method, the target nucleic acids may be mRNA.

### 23. SAMPLES OF MICROPARTICLES OR CELLS

A sample for use in the methods of the invention may comprise at least one microparticle and/or a sample for use in the methods of the invention may be derived from at least one microparticle. The sample may be a mammalian sample. Preferably, the sample is a human sample.

A sample for use in the methods of the invention may comprise at least one cell and/or a sample for use in the methods of the invention may be derived from at least one cell. The sample may be a mammalian sample. Preferably, the sample is a human sample.

The microparticle(s) may be one or more of a variety of cell-free microparticles that have been found in blood, plasma, serum, and other solid/liquid tissue and sample sources from humans and/or other animals (Orozco et al, Cytometry Part A (2010). 77A: 502 514, 2010). “Cell-free” refers to the fact that such microparticles are not cells. Instead, the microparticles are derived from cells e.g. by secretion or following apoptosis. These microparticles are diverse in the tissues and cells from which they originate, as well as the biophysical processes underlying their formation, as well as their respective sizes and molecular structures and compositions. The microparticle may comprise one or more components from a cell membrane (e.g. incorporating phospholipid components) and one or more intracellular and/or cell-nuclear components. The microparticle(s) may be selected from one or more of exosomes, apoptotic bodies (also known as apoptotic vesicles) and/or extracellular microvesicles.

A microparticle may be defined as a membranous vesicle containing at least two fragments of a target nucleic acid (e.g. genomic DNA). A microparticle may have a diameter of 100-5000 nm. Preferably, the microparticle has a diameter of 100-3000 nanometers.

Exosomes are amongst the smallest microparticles, are typically in the range of 50 to 100 nanometers in diameter, and are thought derive from the cell membrane of viable, intact cells, and contain both protein and RNA components (including both mRNA molecules and/or degraded mRNA molecules, and small regulatory RNA molecules such as microRNA molecules) contained within an outer phospholipid component. Exosomes are thought to be formed by exocytosis of cytoplasmic multivesicular bodies (Gyorgy et al, Cell. Mol. Life Sci. (2011) 68:2667-2688). Exosomes are thought to play varied roles in cell-cell signaling as well as extracellular functions (Kanada et al, PNAS (2015) 1418401112). Techniques for quantitating or sequencing the microRNA and/or mRNA molecules found in exosomes have been described previously (e.g. U.S. patent application Ser. No. 13/456,121, European application EP2626433 A1).

Microparticles also include apoptotic bodies (also known as apoptotic vesicles) and extracellular microvesicles, which altogether can range up to 1 micron or even 2 to 5 microns in diameter, and are generally thought to be larger than 100 nanometers in diameter (Lichtenstein et al, Ann N Y Acad Sci. (2001); 945:239-49). All classes of microparticles are thought to be generated by a large number and variety of cells in the body (Thierry et al, Cancer Metastasis Rev 35 (3), 347-376. 9 (2016)/s10555-016-9629-x).

Preferably, the microparticle is not an exosome e.g. the microparticle is any microparticle having a larger diameter than an exosome.

### 24. ISOLATING SAMPLES OF MICROPARTICLES OR CELLS

A large number of methods for isolating microparticles (and/or particular subsets, categories, or fractions of microparticles) have been described previously. European patent(s) ES2540255 (B1) and U.S. Pat. No. 9,005,888 B2 describe methods of isolating particular microparticles such as apoptotic bodies based upon centrifugation procedures. A large number of methods for isolating different types of cell-free microparticles by centrifugation, ultracentrifugation, and other techniques such as nickel-based isolation (e.g using a matrix of beads functionalised with nickel cations to capture vesicles/microparticles), differential centrifugation and/or differential ultracentrifugation, precipitation with hydrophobic agents, chromatography such as ion-exchange chromatography and immunocapture have been well described and developed previously, for example to produce concentrated and/or diffuse solutions of vesicles/microparticles, and/or single-vesicle/single-microparticle suspensions (e.g. without high levels of aggregation of microparticles) (Gyorgy et al, Cell. Mol. Life Sci. (2011) 68:2667-2688; Gardiner et al, J Extracell Vesicles (2016); Momen-Heravi et al, Biol Chem (2013); Deregibus et al, Int J Mol Med (2016); Heath et al, Sci Rep (2018); Notarangelo et al, EBioMedicine (2019))) and may be employed individually or together for any methods described herein.

The cell(s) (or microparticle(s)) may be isolated by centrifugation, size exclusion chromatography and/or filtering.

The step of isolating may comprise centrifugation. The microparticle(s) (or cell(s)) may be isolated by pelleting with a centrifugation step and/or an ultracentrifugation step, or a series of two or more centrifugation steps and/or ultracentrifugation steps at two or more different speeds, wherein the pellet and/or the supernatant from one centrifugation/ultracentrifugation step is further processed in a second centrifugation/ultracentrifugation step, and/or a differential centrifugation process

The centrifugation or ultracentrifugation step(s) may be performed at a speed of 100-500,000 G, 100-1000 G, 1000-10,000 G, 10,000-100,000 G, 500-100,000 G, or 100,000-500,000 G. The centrifugation or ultracentrifugation step may be performed for a duration of at least 5 seconds, at least 10 seconds, at least 30 seconds, at least 60 seconds, at least 5 minutes, at least 10 minutes, at least 30 minutes, at least 60 minutes, or at least 3 hours.

The step of isolating may comprise size exclusion chromatography e.g. a column-based size exclusion chromatography process, such as one including a column comprising a sepharose-based matrix, or a sephacryl-based matrix.

The size exclusion chromatography may comprise using a matrix or filter comprising pore sizes at least 50 nanometers, at least 100 nanometers, at least 200 nanometers, at least 500 nanometers, at least 1.0 micrometer, at least 2.0 micrometers, or at least 5.0 micrometers in size or diameter.

The step of isolating may comprise filtering the sample. The filtrate may provide the microparticle(s) (or cell(s)) analysed in the methods. Optionally, the filter is used to isolate microparticles (or cells) below a certain size, and wherein the filter preferentially or completely removes particles greater than 100 nanometers in size, greater than 200 nanometers in size, greater than 300 nanometers in size, greater than 500 nanometers in size, greater than 1.0 micrometer in size, greater than 2.0 micrometers in size, greater than 3.0 micrometers in size, greater than 5.0 micrometers in size, or greater than 10.0 micrometers in size. Optionally, two or more such filtering steps may be performed, using filters with the same size-filtering parameters, or with different size-filtering parameters. Optionally, the filtrate rom one or more filtering steps comprises microparticles (or cells), and linked sequence reads are produced therefrom.

The sample (e.g. a cell originating from a tissue or organ or tumour sample) may be prepared as a suspension of cells by mechanical homogenization of the tissue or by incubating the tissue in a solution containing dissociation enzymes such as collagenase or trypsin or DNAse or elastase or hyaluronidase. The sample (e.g. a cell from a cell line, or a cell originating from blood, or a cell originating from a pre-implantation embryo generated by in vitro fertilisation or cells from a tissue sample that have been homogenized or dissociated) may be prepared as a suspension of single cells by straining or filtering the cells through a cell strainer or filter with a mesh size of 10 uM or 20 uM or 40 uM or 100 μM. The cell sample may also be prepared as a suspension of single cells of a specific cell type or specific cell types by sorting the cells using methods such as fluorescence-activated cell sorting (FACS) or magnetic-activated cell sorting (MACS) or other methods of isolating specific cell types. The cell sample may also be fixed in methanol prior to use. Dead cells may be removed from the sample by centrifugation or by using commercially available kits such as the MACS dead cell removal kit.

The sample may also comprise nuclei isolated from cells or tissues, such as brain tissue. Here, nuclei may be isolated from the cell or tissue sample by lysis and centrifugation and removal of myelin.

The single cells or nuclei may be resuspended in an isotonic solution such as phosphate-buffered saline (PBS) or Hanks' Balanced salt solution (HBSS), which may contain bovine serum albumin (BSA) or fetal bovine serum (FBS) to reduce cell aggregation at a concentration of at least 0.001% or at least 0.01% or at least 0.1% or at least 1% or at least 10%.

The invention is further defined in the following set of numbered clauses:
1. A library of multimeric barcoding reagents comprising at least 2 multimeric barcoding reagents for labelling target nucleic acids for sequencing, wherein each multimeric barcoding reagent comprises:
   (a) first and second hybridization molecules linked together, wherein each of the hybridization molecules comprises a nucleic acid sequence comprising a hybridization region;
   (b) first and second barcoded oligonucleotides, wherein the first barcoded oligonucleotide is annealed to the hybridization region of the first hybridization molecule and wherein the second barcoded oligonucleotide is annealed to the hybridization region of the second hybridization molecule, wherein the barcoded oligonucleotides each comprise a barcode region; and
   (c) a cell-binding moiety;
   wherein the barcode regions of the first and second barcoded oligonucleotides of a first multimeric barcoding reagent of the library are different to the barcode regions of the first and second barcoded oligonucleotides of a second multimeric barcoding reagent of the library.
2. The method of clause 1, wherein a cell-binding moiety is attached to each of the hybridization molecules.
3. The library of clause 1, wherein the library comprises at least two multimeric barcoding reagents, wherein each multimeric barcoding reagent comprises:
   (a) first and second barcode molecules linked together, wherein each of the barcode molecules comprises a nucleic acid sequence comprising a barcode region;
   (b) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule; and
   (c) a cell-binding moiety;
   wherein the barcode regions of the first and second barcoded oligonucleotides of a first multimeric barcoding reagent of the library are different to the barcode regions of the first and second barcoded oligonucleotides of a second multimeric barcoding reagent of the library.
4. The library of clause 3, wherein a cell-binding moiety is attached to each of the barcode molecules.
5. The library of any one of clauses 1-4, wherein a cell-binding moiety is attached to each of the barcoded oligonucleotides.
6. A kit for labelling target nucleic acids for sequencing, wherein the kit comprises:
   (a) a library of multimeric barcoding reagents comprising at least two multimeric barcoding reagents, wherein each multimeric barcoding reagent comprises
      (i) first and second barcode molecules linked together, wherein each of the barcode molecules comprises a nucleic acid sequence comprising, optionally in the 5′ to 3′ direction, an adapter region and a barcode region,
      (ii) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule;
      wherein the barcode regions of the first and second barcoded oligonucleotides of a first multimeric barcoding reagent of the library are different to the barcode regions of the first and second barcoded oligonucleotides of a second multimeric barcoding reagent of the library; and
   (b) first and second adapter oligonucleotides for each of the multimeric barcoding reagents, wherein the first adapter oligonucleotide comprises an adapter region capable of annealing to the adapter region of the first barcode molecule and wherein the second adapter oligonucleotide comprises an adapter region capable of annealing to the adapter region of the second barcode molecule, and wherein a cell-binding moiety is attached to each of the adapter oligonucleotides.
7. The kit of clause 6, wherein the multimeric barcoding reagents each comprise a cell-binding moiety.
8. The kit of clause 7, wherein a cell-binding moiety is attached to each of the barcode molecules.
9. The kit of clause 7 or clause 8, wherein a cell-binding moiety is attached to each of the barcoded oligonucleotides.
10. The library of any one of clauses 1-5, wherein the first multimeric barcoding reagent is comprised within a first lipid carrier and the second multimeric barcoding reagent is comprised within a second lipid carrier.
11. The kit of any one of clauses 6-9, wherein the first and second adapter oligonucleotides for the first multimeric barcoding reagent are comprised within a first lipid carrier and the first and second adapter oligonucleotides for the second multimeric barcoding reagent are comprised within a second lipid carrier.
12. The kit of clause 11, wherein the first lipid carrier further comprises the first multimeric barcoding reagent and wherein the second lipid carrier further comprises the second multimeric barcoding reagent.
13. The library or kit of any one of clauses 10-12, wherein the lipid carrier is a liposome or a micelle.
14. The library or kit of any one of clauses 1-13, wherein the multimeric barcoding reagents each comprise a solid support or semi-solid support, and wherein a cell-binding moiety is attached to the solid support.
15. The library or kit of any one of clauses 1-14, wherein a cell-binding moiety is attached to each barcoded oligonucleotide, hybridization molecule, barcode molecule and/or adapter oligonucleotide by a linker molecule.
16. The library of kit of any one of clauses 1-15, wherein the cell-binding moiety is capable of initiating endocytosis on binding to a cell membrane.
17. The library or kit of any one of clauses 1-16, wherein the cell-binding moiety comprises one or more moieties selected from: a peptide, a cell penetrating peptide, an aptamer, a DNA adptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a cationic lipid, a cationic polymer, poly(ethylene) glycol, spermine, a spermine derivatives or analogue, a poly-lysine, a poly-lysine derivative or analogue, polyethyleneimine, diethylaminoethyl (DEAE)-dextran, cholesterol, a sterol moiety, a cationic molecule, a hydrophobic molecule and an amphiphilic molecule.
18. A method of preparing a nucleic acid sample for sequencing, wherein the sample comprises at least two cells, and wherein the method comprises the steps of:
   (a) contacting the sample with a library comprising at least two multimeric barcoding reagents, wherein each multimeric barcoding reagent comprises first and second barcode regions linked together and a cell-binding moiety, wherein each barcode region comprises a nucleic acid sequence and wherein the first and second barcode regions of a first multimeric barcoding reagent are different to the first and second barcode regions of a second multimeric barcoding reagent of the library, wherein the cell-binding moiety of the first multimeric barcoding reagent from the library binds to the cell membrane of a first cell of the sample and the first and second barcode regions of the first multimeric barcoding reagent are internalized into the first cell, and wherein the cell-binding moiety of the second multimeric barcoding reagent from the library binds to the cell membrane of a second cell of the sample and the first and second barcode regions of the second multimeric barcoding reagent are internalized into the second cell; and
   (b) appending barcode sequences to each of first and second sub-sequences of a target nucleic acid of the first cell to produce first and second barcoded target nucleic acid molecules for the first cell, wherein the first barcoded target nucleic acid molecule comprises the nucleic acid sequence of the first barcode region of the first multimeric barcoding reagent and the second barcoded target nucleic acid molecule comprises the nucleic acid sequence of the second barcode region of the first multimeric barcoding reagent, and appending barcode sequences to each of first and second sub-sequences of a target nucleic acid of the second cell to produce first and second barcoded target nucleic acid molecules for the second cell, wherein the first barcoded target nucleic acid molecule comprises the nucleic acid sequence of the first barcode region of the second multimeric barcoding reagent and the second barcoded target nucleic acid molecule comprises the nucleic acid sequence of the second barcode region of the second multimeric barcoding reagent.
19. The method of clause 18, wherein the method comprises the steps of:
   (a) contacting the sample with a library comprising at least two multimeric barcoding reagents, wherein each multimeric barcoding reagent comprises first and second barcoded oligonucleotides linked together and a cell-binding moiety, wherein the barcoded oligonucleotides each comprise a barcode region and wherein the barcode regions of the first and second barcoded oligonucleotides of a first multimeric barcoding reagent of the library are different to the barcode regions of the first and second barcoded oligonucleotides of a second multimeric barcoding reagent of the library, wherein the cell-binding moiety of a first multimeric barcoding reagent from the library binds to the cell membrane of a first cell of the sample and the first and second barcoded oligonucleotides of the first multimeric barcoding reagent are internalized into the first cell, and wherein the cell-binding moiety of a second multimeric barcoding reagent from the library binds to the cell membrane of a second cell of the sample and the first and second barcoded oligonucleotides of the second multimeric barcoding reagent are internalized into the second cell; and
   (b) annealing or ligating the first and second barcoded oligonucleotides of the first multimeric barcoding reagent to first and second sub-sequences of a target nucleic acid of the first cell to produce first and second barcoded target nucleic acid molecules, and annealing or ligating the first and second barcoded oligonucleotides from the second multimeric barcoding reagent to first and second sub-sequences of a target nucleic acid of the second cell to produce first and second barcoded target nucleic acid molecules.
20. The method of clause 18, wherein step (b) comprises:
   (i) annealing the first and second barcoded oligonucleotides of the first multimeric barcoding reagent to first and second sub-sequences of a target nucleic acid of the first cell, and annealing the first and second barcoded oligonucleotides of the second multimeric barcoding reagent to first and second sub-sequences of a target nucleic acid of the second cell; and
   (ii) extending the first and second barcoded oligonucleotides of the first multimeric barcoding reagent to produce first and second different barcoded target nucleic acid molecules and extending the first and second barcoded oligonucleotides of the second multimeric barcoding reagent to produce first and second different barcoded target nucleic acid molecules, wherein each of the barcoded target nucleic acid molecules comprises at least one nucleotide synthesised from the target nucleic acid as a template.
21. The method of any one of clauses 18-20, wherein a cell-binding moiety is attached to each of the barcoded oligonucleotides.
22. The method of any one of clauses 18-21, wherein the multimeric barcoding reagents each comprise:
   (i) first and second hybridization molecules linked together, wherein each of the hybridization molecules comprises a nucleic acid sequence comprising a hybridization region; and
   (ii) first and second barcoded oligonucleotides, wherein the first barcoded oligonucleotide is annealed to the hybridization region of the first hybridization molecule and wherein the second barcoded oligonucleotide is annealed to the hybridization region of the second hybridization molecule;
   optionally wherein the first multimeric barcoding reagent is internalized into the first cell and the second multimeric barcoding reagent is internalized into the second cell.
23. The method of clause 22, wherein a cell-binding moiety is attached to each of the hybridization molecules.
24. The method of clause 22, wherein the multimeric barcoding reagents each comprise:
   (i) first and second barcode molecules linked together, wherein each of the barcode molecules comprises a nucleic acid sequence comprising a barcode region; and
   (ii) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule;
   optionally wherein the first multimeric barcoding reagent is internalized into the first cell and the second multimeric barcoding reagent is internalized into the second cell.
25. The method of clause 24, wherein a cell-binding moiety is attached to each of the barcode molecules.
26. A method of preparing a nucleic acid sample for sequencing, wherein the sample comprises at least two cells, and wherein the method comprises the steps of:
   (a) contacting the sample with a library comprising first and second multimeric barcoding reagents, wherein each multimeric barcoding reagent comprises:
      (i) first and second barcode molecules linked together, wherein each of the barcode molecules comprises a nucleic acid sequence comprising, optionally in the 5′ to 3′ direction, an adapter region and a barcode region, and
      (ii) first and second barcoded oligonucleotides, wherein the first barcoded oligonucleotide comprises a barcode region annealed to the barcode region of the first barcode molecule and wherein the second barcoded oligonucleotide comprises a barcode region annealed to the barcode region of the second barcode molecule, and wherein the barcode regions of the first and second barcoded oligonucleotides of the first multimeric barcoding reagent of the library are different to the barcode regions of the first and second barcoded oligonucleotides of the second multimeric barcoding reagent of the library;
      wherein the sample is further contacted with first and second adapter oligonucleotides for each of the multimeric barcoding reagents, wherein the first and second adapter oligonucleotides each comprise an adapter region, wherein a cell-binding moiety is attached to each of the adapter oligonucleotides, and wherein the cell-binding moieties of the first and second adapter oligonucleotides for the first multimeric barcoding reagent bind to the cell membrane of a first cell of the sample and the first and second adapter oligonucleotides for the first multimeric barcoding reagent are internalized into the first cell, and wherein the cell-binding moieties of the first and second adapter oligonucleotides for the second multimeric barcoding reagent bind to the cell membrane of a second cell of the sample and the first and second adapter oligonucleotides for the second multimeric barcoding reagent are internalized into the second cell;
   (b) annealing or ligating the first and second adapter oligonucleotides for the first multimeric barcoding reagent to sub-sequences of a target nucleic acid of the first cell, and annealing or ligating the first and second adapter oligonucleotides for the second multimeric barcoding reagent to sub-sequences of a target nucleic acid of the second cell;
   (c) for each of the multimeric barcoding reagents, annealing the adapter region of the first adapter oligonucleotide to the adapter region of the first barcode molecule, and annealing the adapter region of the second adapter oligonucleotide to the adapter region of the second barcode molecule; and
   (d) for each of the multimeric barcoding reagents, ligating the 3′ end of the first barcoded oligonucleotide to the 5′ end of the first adapter oligonucleotide to produce a first barcoded target nucleic acid molecule and ligating the 3′ end of the second barcoded oligonucleotide to the 5′ end of the second adapter oligonucleotide to produce a second barcoded target nucleic acid molecule.
27. The method of clause 26, wherein step (b) comprises annealing the first and second adapter oligonucleotides for the first multimeric barcoding reagent to sub-sequences of a target nucleic acid of the first cell, and annealing the first and second adapter oligonucleotides for the second multimeric barcoding reagent to sub-sequences of a target nucleic acid of the second cell, and wherein either:
   (i) for each of the multimeric barcoding reagents, step (d) comprises ligating the 3′ end of the first barcoded oligonucleotide to the 5′ end of the first adapter oligonucleotide to produce a first barcoded-adapter oligonucleotide and ligating the 3′ end of the second barcoded oligonucleotide to the 5′ end of the second adapter oligonucleotide to produce a second barcoded-adapter oligonucleotide, and extending the first and second barcoded-adapter oligonucleotides to produce first and second different barcoded target nucleic acid molecules each of which comprises at least one nucleotide synthesised from the target nucleic acid as a template, or
   (ii) for each of the multimeric barcoding reagents, before step (d), the method comprises extending the first and second adapter oligonucleotides to produce first and second different target nucleic acid molecules each of which comprises at least one nucleotide synthesised from the target nucleic acid as a template.
28. The method of clause 27, wherein the multimeric barcoding reagents each comprise a cell-binding moiety, optionally wherein:
   (i) the cell-binding moiety of the first multimeric barcoding reagent binds to the cell membrane of the first cell of the sample and the multimeric barcoding reagent is internalized into the first cell and
   (ii) the cell-binding moiety of the second multimeric barcoding reagent binds to the cell membrane of the second cell of the sample and the second multimeric barcoding reagent is internalized into the second cell.
29. The method of clause 28, wherein a cell-binding moiety is attached to each of the barcode molecules.
30. The method of clause 28 or clause 29, wherein a cell-binding moiety is attached to each of the barcoded oligonucleotides.
31. The method of any one of clauses 18-25, wherein the first multimeric barcoding reagent is comprised within a first lipid carrier and the second multimeric barcoding reagent is comprised within a second lipid carrier, optionally wherein in step (a) the first lipid carrier merges with the cell membrane of the first cell and the first and second barcoded oligonucleotides of the first multimeric barcoding reagent are internalized into the first cell, and the second lipid carrier merges with the cell membrane of the second cell and the first and second barcoded oligonucleotides of the first multimeric barcoding reagent are internalized into the second cell.
32. The method of any one of clauses 26-30, wherein the first and second adapter oligonucleotides for the first multimeric barcoding reagent are comprised within a first lipid carrier and the first and second adapter oligonucleotides for the second multimeric barcoding reagent are comprised within a second lipid carrier, optionally wherein in step (a) the first lipid carrier merges with the cell membrane of the first cell and the first and second adapter oligonucleotides for the first multimeric barcoding reagent are internalized into the first cell, and the second lipid carrier merges with the cell membrane of the second cell and the first and second adapter oligonucleotides for the second multimeric barcoding reagent are internalized into the second cell.
33. The method of clause 32, wherein the first lipid carrier further comprises the first multimeric barcoding reagent and wherein the second lipid carrier further comprises the second multimeric barcoding reagent.
34. The method of any one of clauses 31-33, wherein the lipid carrier is a liposome or micelle.
35. The method of any one of clauses 18-34, wherein the multimeric barcoding reagents each comprise a solid support or semi-solid support, and wherein a cell-binding moiety is attached to the solid support.
36. The method of any one of clauses 18-35, wherein a cell-binding moiety is attached to each barcoded oligonucleotide, hybridization molecule, barcode molecule and/or adapter oligonucleotide by a linker molecule.
37. The method of any one of clauses 18-36, wherein the multimeric barcoding reagents and/or adapter oligonucleotides are internalized by endocytosis.
38. The method of any one of clauses 18-37, wherein the cell-binding moiety comprises one or more moieties selected from: a peptide, a cell penetrating peptide, an aptamer, a DNA adptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a cationic lipid, a cationic polymer, poly(ethylene) glycol, spermine, a spermine derivatives or analogue, a poly-lysine, a poly-lysine derivative or analogue, polyethyleneimine, diethylaminoethyl (DEAE)-dextran, cholesterol, a sterol moiety, a cationic molecule, a hydrophobic molecule and an amphiphilic molecule.
39. A method of preparing a nucleic acid sample for sequencing, wherein the sample comprises at least two cells, and wherein the method comprises the steps of:
   (a) contacting the sample with a library comprising at least two multimeric barcoding reagents, wherein each multimeric barcoding reagent comprises first and second barcode regions linked together, wherein each barcode region comprises a nucleic acid sequence and wherein the first and second barcode regions of a first multimeric barcoding reagent are different to the first and second barcode regions of a second multimeric barcoding reagent of the library;
   (b) transferring the first and second barcode regions of the first multimeric barcoding reagent from the library into a first cell of the sample and transferring the first and second barcode regions of the second multimeric barcoding reagent from the library into a second cell of the sample; and
   (c) appending barcode sequences to each of first and second sub-sequences of a target nucleic acid of the first cell to produce first and second barcoded target nucleic acid molecules for the first cell, wherein the first barcoded target nucleic acid molecule comprises the nucleic acid sequence of the first barcode region of the first multimeric barcoding reagent and the second barcoded target nucleic acid molecule comprises the nucleic acid sequence of the second barcode region of the first multimeric barcoding reagent, and appending barcode sequences to each of first and second sub-sequences of a target nucleic acid of the second cell to produce first and second barcoded target nucleic acid molecules for the second cell, wherein the first barcoded target nucleic acid molecule comprises the nucleic acid sequence of the first barcode region of the second multimeric barcoding reagent and the second barcoded target nucleic acid molecule comprises the nucleic acid sequence of the second barcode region of the second multimeric barcoding reagent.
40. The method of clause 39, wherein the method comprises the steps of:
   (a) contacting the sample with a library comprising at least two multimeric barcoding reagents, wherein each multimeric barcoding reagent comprises first and second barcoded oligonucleotides linked together, wherein the barcoded oligonucleotides each comprise a barcode region and wherein the barcode regions of the first and second barcoded oligonucleotides of a first multimeric barcoding reagent of the library are different to the barcode regions of the first and second barcoded oligonucleotides of a second multimeric barcoding reagent of the library;
   (b) transferring the first and second barcoded oligonucleotides of the first multimeric barcoding reagent from the library into a first cell of the sample and transferring the first and second barcoded oligonucleotides of the second multimeric barcoding reagent from the library into a second cell of the sample; and
   (c) annealing or ligating the first and second barcoded oligonucleotides of the first multimeric barcoding reagent to first and second sub-sequences of a target nucleic acid of the first cell to produce first and second barcoded target nucleic acid molecules, and annealing or ligating the first and second barcoded oligonucleotides from the second multimeric barcoding reagent to first and second sub-sequences of a target nucleic acid of the second cell to produce first and second barcoded target nucleic acid molecules.
41. The method of clause 40, wherein step (c) comprises:
   (i) annealing the first and second barcoded oligonucleotides of the first multimeric barcoding reagent to first and second sub-sequences of a target nucleic acid of the first cell, and annealing the first and second barcoded oligonucleotides of the second multimeric barcoding reagent to first and second sub-sequences of a target nucleic acid of the second cell; and
   (ii) extending the first and second barcoded oligonucleotides of the first multimeric barcoding reagent to produce first and second different barcoded target nucleic acid molecules and extending the first and second barcoded oligonucleotides of the second multimeric barcoding reagent to produce first and second different barcoded target nucleic acid molecules, wherein each of the barcoded target nucleic acid molecules comprises at least one nucleotide synthesised from the target nucleic acid as a template.
42. The method of any one of clauses 39-41, wherein the multimeric barcoding reagents each comprise:
   (i) first and second hybridization molecules linked together, wherein each of the hybridization molecules comprises a nucleic acid sequence comprising a hybridization region; and
   (ii) first and second barcoded oligonucleotides, wherein the first barcoded oligonucleotide is annealed to the hybridization region of the first hybridization molecule and wherein the second barcoded oligonucleotide is annealed to the hybridization region of the second hybridization molecule;
   optionally wherein step (b) comprises transferring the first multimeric barcoding reagent into the first cell and transferring the second multimeric barcoding reagent into the second cell.
43. The method of clause 42, wherein the multimeric barcoding reagents each comprise:
   (i) first and second barcode molecules linked together, wherein each of the barcode molecules comprises a nucleic acid sequence comprising a barcode region; and
   (ii) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule;
   optionally wherein step (b) comprises transferring the first multimeric barcoding reagent into the first cell and transferring the second multimeric barcoding reagent into the second cell.
44. A method of preparing a nucleic acid sample for sequencing, wherein the sample comprises at least two cells, and wherein the method comprises the steps of:
   (a) contacting the sample with a library comprising first and second multimeric barcoding reagents, wherein each multimeric barcoding reagent comprises:
      (i) first and second barcode molecules linked together, wherein each of the barcode molecules comprises a nucleic acid sequence comprising, optionally in the 5′ to 3′ direction, an adapter region and a barcode region, and
      (ii) first and second barcoded oligonucleotides, wherein the first barcoded oligonucleotide comprises a barcode region annealed to the barcode region of the first barcode molecule, wherein the second barcoded oligonucleotide comprises a barcode region annealed to the barcode region of the second barcode molecule, and wherein the barcode regions of the first and second barcoded oligonucleotides of the first multimeric barcoding reagent of the library are different to the barcode regions of the first and second barcoded oligonucleotides of the second multimeric barcoding reagent of the library;
      wherein the sample is further contacted with first and second adapter oligonucleotides for each of the multimeric barcoding reagents, wherein the first and second adapter oligonucleotides each comprise an adapter region;
   (b) transferring the first and second adapter oligonucleotides for the first multimeric barcoding reagent into the first cell and transferring the first and second adapter oligonucleotides for the second multimeric barcoding reagent into the second cell, optionally wherein the step further comprises transferring the first multimeric barcoding reagent into the first cell and transferring the second multimeric barcoding reagent into the second cell;
   (c) annealing or ligating the first and second adapter oligonucleotides for the first multimeric barcoding reagent to sub-sequences of a target nucleic acid of the first cell, and annealing or ligating the first and second adapter oligonucleotides for the second multimeric barcoding reagent to sub-sequences of a target nucleic acid of the second cell;
   (d) for each of the multimeric barcoding reagents, annealing the adapter region of the first adapter oligonucleotide to the adapter region of the first barcode molecule, and annealing the adapter region of the second adapter oligonucleotide to the adapter region of the second barcode molecule; and
   (e) for each of the multimeric barcoding reagents, ligating the 3′ end of the first barcoded oligonucleotide to the 5′ end of the first adapter oligonucleotide to produce a first barcoded target nucleic acid molecule and ligating the 3′ end of the second barcoded oligonucleotide to the 5′ end of the second adapter oligonucleotide to produce a second barcoded target nucleic acid molecule.
45. The method of clause 44, wherein step (c) comprises annealing the first and second adapter oligonucleotides for the first multimeric barcoding reagent to sub-sequences of a target nucleic acid of the first cell, and annealing the first and second adapter oligonucleotides for the second multimeric barcoding reagent to sub-sequences of a target nucleic acid of the second cell, and wherein either:
   (i) for each of the multimeric barcoding reagents, step (e) comprises ligating the 3′ end of the first barcoded oligonucleotide to the 5′ end of the first adapter oligonucleotide to produce a first barcoded-adapter oligonucleotide and ligating the 3′ end of the second barcoded oligonucleotide to the 5′ end of the second adapter oligonucleotide to produce a second barcoded-adapter oligonucleotide, and extending the first and second barcoded-adapter oligonucleotides to produce first and second different barcoded target nucleic acid molecules each of which comprises at least one nucleotide synthesised from the target nucleic acid as a template, or
   (ii) for each of the multimeric barcoding reagents, before step (e), the method comprises extending the first and second adapter oligonucleotides to produce first and second different target nucleic acid molecules each of which comprises at least one nucleotide synthesised from the target nucleic acid as a template.
46. The method of any one of clauses 39-45, wherein prior to step (b), the cell membrane of the cells are permeabilised by contact with a chemical surfactant.
47. The method of any one of clauses 39-46, wherein prior to step (b), the cell membrane of the cells are permeabilised by contact with a solvent.
48. The method of any one of clauses 39-47, wherein the barcoded oligonucleotides, adapter oligonucleotides and/or multimeric barcoding reagents are transferred into the cells by complexation with a transfection reagent or lipid carrier.
49. The method of any one of clauses 39-48, wherein the barcoded oligonucleotides of the first multimeric barcoding reagent are comprised within a first lipid carrier, and wherein the barcoded oligonucleotides of the second multimeric barcoding reagent are comprised within a second lipid carrier.
50. The method of any one of clauses 39-49, wherein the barcoded oligonucleotides, adapter oligonucleotides and/or multimeric barcoding reagents are transferred into the cells by a process comprising cell squeezing.
51. The method of any one of clauses 39-50, wherein the barcoded oligonucleotides, adapter oligonucleotides and/or multimeric barcoding reagents are transferred into the cells by a process comprising electroporation.
52. A method of preparing a nucleic acid sample for sequencing, wherein the sample comprises at least two cells, and wherein the method comprises the steps of:
   (a) contacting the sample with a library comprising at least two multimeric barcoding reagents, wherein each multimeric barcoding reagent comprises first and second barcode regions linked together, wherein each barcode region comprises a nucleic acid sequence and wherein the first and second barcode regions of a first multimeric barcoding reagent are different to the first and second barcode regions of a second multimeric barcoding reagent of the library;
   (b) lysing the cells or permeabilizing the cell membranes of the cells; and
   (c) appending barcode sequences to each of first and second sub-sequences of a target nucleic acid of the first cell to produce first and second barcoded target nucleic acid molecules for the first cell, wherein the first barcoded target nucleic acid molecule comprises the nucleic acid sequence of the first barcode region of the first multimeric barcoding reagent and the second barcoded target nucleic acid molecule comprises the nucleic acid sequence of the second barcode region of the first multimeric barcoding reagent, and appending barcode sequences to each of first and second sub-sequences of a target nucleic acid of the second cell to produce first and second barcoded target nucleic acid molecules for the second cell, wherein the first barcoded target nucleic acid molecule comprises the nucleic acid sequence of the first barcode region of the second multimeric barcoding reagent and the second barcoded target nucleic acid molecule comprises the nucleic acid sequence of the second barcode region of the second multimeric barcoding reagent.
53. The method of clause 52, wherein the method comprises the steps of:
   (a) contacting the sample with a library comprising at least two multimeric barcoding reagents, wherein each multimeric barcoding reagent comprises first and second barcoded oligonucleotides linked together, wherein the barcoded oligonucleotides each comprise a barcode region and wherein the barcode regions of the first and second barcoded oligonucleotides of a first multimeric barcoding reagent of the library are different to the barcode regions of the first and second barcoded oligonucleotides of a second multimeric barcoding reagent of the library;
   (b) lysing the cells or permeabilizing the cell membranes of the cells; and
   (c) annealing or ligating the first and second barcoded oligonucleotides of the first multimeric barcoding reagent to first and second sub-sequences of a target nucleic acid of the first cell to produce first and second barcoded target nucleic acid molecules, and annealing or ligating the first and second barcoded oligonucleotides from the second multimeric barcoding reagent to first and second sub-sequences of a target nucleic acid of the second cell to produce first and second barcoded target nucleic acid molecules.
54. The method of clause 52, wherein step (c) comprises:
   (i) annealing the first and second barcoded oligonucleotides of the first multimeric barcoding reagent to first and second sub-sequences of a target nucleic acid of the first cell, and annealing the first and second barcoded oligonucleotides of the second multimeric barcoding reagent to first and second sub-sequences of a target nucleic acid of the second cell; and
   (ii) extending the first and second barcoded oligonucleotides of the first multimeric barcoding reagent to produce first and second different barcoded target nucleic acid molecules and extending the first and second barcoded oligonucleotides of the second multimeric barcoding reagent to produce first and second different barcoded target nucleic acid molecules, wherein each of the barcoded target nucleic acid molecules comprises at least one nucleotide synthesised from the target nucleic acid as a template.
55. The method of any one of clauses 52-54, wherein the multimeric barcoding reagents each comprise:
   (i) first and second hybridization molecules linked together, wherein each of the hybridization molecules comprises a nucleic acid sequence comprising a hybridization region; and
   (ii) first and second barcoded oligonucleotides, wherein the first barcoded oligonucleotide is annealed to the hybridization region of the first hybridization molecule and wherein the second barcoded oligonucleotide is annealed to the hybridization region of the second hybridization molecule.
56. The method of clause 55, wherein the multimeric barcoding reagents each comprise:
   (i) first and second barcode molecules linked together, wherein each of the barcode molecules comprises a nucleic acid sequence comprising a barcode region; and
   (ii) first and second barcoded oligonucleotides, wherein the first barcoded oligonucleotide comprises a barcode region annealed to the barcode region of the first barcode molecule, and wherein the second barcoded oligonucleotide comprises a barcode region annealed to the barcode region of the second barcode molecule.
57. A method of preparing a nucleic acid sample for sequencing, wherein the sample comprises at least two cells, and wherein the method comprises the steps of:
   (a) contacting the sample with a library comprising first and second multimeric barcoding reagents, wherein each multimeric barcoding reagent comprises:
      (i) first and second barcode molecules linked together, wherein each of the barcode molecules comprises a nucleic acid sequence comprising, optionally in the 5′ to 3′ direction, an adapter region and a barcode region, and
      (ii) first and second barcoded oligonucleotides, wherein the first barcoded oligonucleotide comprises a barcode region annealed to the barcode region of the first barcode molecule and wherein the second barcoded oligonucleotide comprises a barcode region annealed to the barcode region of the second barcode molecule, and wherein the barcode regions of the first and second barcoded oligonucleotides of the first multimeric barcoding reagent are different to the barcode regions of the first and second barcoded oligonucleotides of the second multimeric barcoding reagent;
      wherein the sample is further contacted with first and second adapter oligonucleotides for each of the multimeric barcoding reagents, wherein the first and second adapter oligonucleotides each comprise an adapter region;
   (b) lysing the cells or permeabilizing the cell membranes of the cells;
   (c) annealing or ligating the first and second adapter oligonucleotides for the first multimeric barcoding reagent to sub-sequences of a target nucleic acid of the first cell, and annealing or ligating the first and second adapter oligonucleotides for the second multimeric barcoding reagent to sub-sequences of a target nucleic acid of the second cell;
   (d) for each of the multimeric barcoding reagents, annealing the adapter region of the first adapter oligonucleotide to the adapter region of the first barcode molecule, and annealing the adapter region of the second adapter oligonucleotide to the adapter region of the second barcode molecule; and
   (e) for each of the multimeric barcoding reagents, ligating the 3′ end of the first barcoded oligonucleotide to the 5′ end of the first adapter oligonucleotide to produce a first barcoded target nucleic acid molecule and ligating the 3′ end of the second barcoded oligonucleotide to the 5′ end of the second adapter oligonucleotide to produce a second barcoded target nucleic acid molecule.
58. The method of clause 57, wherein step (c) comprises annealing the first and second adapter oligonucleotides for the first multimeric barcoding reagent to sub-sequences of a target nucleic acid of the first cell, and annealing the first and second adapter oligonucleotides for the second multimeric barcoding reagent to sub-sequences of a target nucleic acid of the second cell, and wherein either:
   (i) for each of the multimeric barcoding reagents, step (e) comprises ligating the 3′ end of the first barcoded oligonucleotide to the 5′ end of the first adapter oligonucleotide to produce a first barcoded-adapter oligonucleotide and ligating the 3′ end of the second barcoded oligonucleotide to the 5′ end of the second adapter oligonucleotide to produce a second barcoded-adapter oligonucleotide, and extending the first and second barcoded-adapter oligonucleotides to produce first and second different barcoded target nucleic acid molecules each of which comprises at least one nucleotide synthesised from the target nucleic acid as a template, or
   (ii) for each of the multimeric barcoding reagents, before step (e), the method comprises extending the first and second adapter oligonucleotides to produce first and second different target nucleic acid molecules each of which comprises at least one nucleotide synthesised from the target nucleic acid as a template.
59. The method of any one of clauses 52-58, wherein following step (b) target nucleic acids from each cell within the sample are able to diffuse out of the cell.
60. The method of any one of clauses 52-59, wherein step (b) is performed by increasing the temperature of the sample.
61. The method of any one of clauses 52-60, wherein step (b) is performed in the presence of a chemical surfactant.
62. The method of any one of clauses 52-61, wherein step (b) is performed in the presence of a solvent.
63. The method of any one of clauses 52-62, wherein step (b) is performed under hypotonic or hypertonic conditions.
64. The method of any one of clauses 52-63, wherein the multimeric barcoding reagents and/or adapter oligonucleotides each comprise a cell-binding moiety, optionally wherein the cell-binding moiety binds each multimeric barcoding reagent and/or adapter oligonucleotide to the cell membrane of the cells prior to step (b).
65. The method of any one of clauses 18-64, wherein the target nucleic acids are mRNA.
66. A multimeric barcoding reagent for labelling a target nucleic acid for sequencing, wherein the multimeric barcoding reagent comprises:
   a. a support;
   b. at least two multimeric hybridization molecules, wherein each multimeric hybridization molecule is independently linked to the support and wherein each multimeric hybridization molecule comprises at least two hybridization molecules linked together, wherein each of the hybridization molecules comprises a nucleic acid sequence comprising a hybridization region;
   c. at least two barcoded oligonucleotides annealed to each of the multimeric hybridization molecules, wherein each barcoded oligonucleotide is annealed to one of the hybridization regions and wherein each barcoded oligonucleotide comprises a barcode region; and
   d. a cell-binding moiety linked to each multimeric hybridization molecule.
67. The multimeric barcoding reagent of clause 66, wherein the hybridization molecules of each multimeric hybridization molecule are linked on a nucleic acid molecule.
68. The multimeric barcoding reagent of clause 66 or clause 67, wherein the hybridization molecules of each multimeric hybridization molecule are linked on a linear nucleic acid molecule.
69. The multimeric barcoding reagent of any one of clauses 66-68, wherein the first end of each linear nucleic acid molecule is linked to the support and the second end is linked to a cell-binding moiety.
70. The multimeric barcoding reagent of any one of clauses 66-69, wherein each cell-binding moiety is linked to one of the multimeric hybridization molecules by a cell-binding oligonucleotide.
71. The multimeric barcoding reagent of any one of clauses 66-70, wherein each cell-binding oligonucleotide is annealed to one of the multimeric hybridization molecules.
72. The multimeric barcoding reagent of any one of clauses 66-71, wherein each barcoded oligonucleotide comprises, optionally in the 5′ to 3′ direction, an adapter region annealed to one of the hybridization regions, a barcode region, and a target region capable of annealing or ligating to a sub-sequence of the target nucleic acid.
73. The multimeric barcoding reagent of any one of clauses 66-71, wherein each barcoded oligonucleotide comprises, optionally in the 5′ to 3′ direction, a barcode region, an adapter region annealed to one of the hybridization regions and a target region capable of annealing or ligating to a sub-sequence of the target nucleic acid.
74. The multimeric barcoding reagent of any one of clauses 66-73, wherein the adapter regions of the barcoded oligonucleotides of the multimeric barcoding reagent are identical.
75. The multimeric barcoding reagent of any one of clauses 66-74, wherein each multimeric hybridization molecule comprises at least 2, at least 3, at least 5, at least 10, at least 20, at least 50, at least 100, at least 200, at least 500, at least 1000, at least 5000, at least 10⁴, at least 10⁵, at least 10⁶, at least 10⁷, at least 10⁸, at least 10⁹ or at least 10¹⁰ hybridization molecules linked together, wherein each of the hybridization molecules comprises a nucleic acid sequence comprising a hybridization region.
76. The multimeric barcoding reagent of any one of clauses 66-75, wherein the multimeric barcoding reagent comprises a barcoded oligonucleotide for each of the hybridization regions, and wherein each barcoded oligonucleotide is annealed to one of the hybridization regions.
77. The multimeric barcoding reagent of any one of clauses 66-76, wherein the multimeric barcoding reagent comprises at least 2, at least 3, at least 5, at least 10, at least 20, at least 50, at least 100, at least 200, at least 500, at least 1000, at least 5000, at least 10⁴, at least 10⁵, at least 10⁶, at least 10⁷, at least 10⁸, at least 10⁹, or at least 10¹⁰ barcoded oligonucleotides annealed to each of the multimeric hybridization molecules, wherein each barcoded oligonucleotide is annealed to one of the hybridization regions and wherein each barcoded oligonucleotide comprises a barcode region.
78. The multimeric barcoding reagent of any one of clauses 66-77, wherein the multimeric barcoding reagent comprises at least 2, at least 3, at least 5, at least 10, at least 20, at least 50, at least 100, at least 200, at least 500, at least 1000, at least 5000, at least 10⁴, at least 10⁵, at least 10⁶, at least 10⁷, at least 10⁸, at least 10⁹, or at least 10¹⁰ barcoded oligonucleotides with identical barcode regions.
79. The multimeric barcoding reagent of any one of clauses 66-78, wherein the multimeric barcoding reagent comprises at least 3, at least 4, at least 5, at least 10, at least 20, at least 25, at least 50, at least 75, at least 100, at least 200, at least 500, at least 1000, at least 5000, at least 10⁴, at least 10⁵, at least 10⁶, at least 10⁷, at least 10⁸, at least 10⁹, or at least 10¹⁰ multimeric hybridization molecules, wherein each multimeric hybridization molecule is as defined in any one of clauses 66-78.
80. A library of multimeric barcoding reagents comprising at least 2, at least 5, at least 10, at least 20, at least 25, at least 50, at least 75, at least 100, at least 250, at least 500, at least 10³, at least 10⁴, at least 10⁵, at least 10⁶, at least 10⁷, at least 10⁸, at least 10⁹, at least 10¹⁰ multimeric barcoding reagents, wherein each multimeric barcoding reagent is as defined in any one of clauses 66-79.
81. The library of multimeric barcoding reagents of clause 80, wherein at least 50%, at least 60%, at least 70%, at least 75%, at least 80%, at least 85%, at least 90%, at least 95%, at least 99%, at least 99.9%, at least 99.99%, at least 99.999%, at least 99.9999%, or 100% of the barcode regions of each multimeric barcoding reagent are different to the barcode regions of the other multimeric barcoding reagents in the library.
82. A method of preparing a nucleic acid sample for sequencing, wherein the sample comprises at least 2 cells, and wherein the method comprises in order the steps of:
   (a) contacting the sample with a library comprising at least two multimeric barcoding reagents, wherein each multimeric barcoding reagent is as defined in any one of clauses 66-79, wherein the barcode regions of the barcoded oligonucleotides of a first multimeric barcoding reagent of the library are different to the barcode regions of the barcoded oligonucleotides of a second multimeric barcoding reagent of the library, wherein the cell-binding moiety of the first multimeric barcoding reagent binds to the cell membrane of a first cell prior to step (b), and wherein the cell-binding moiety of the second multimeric barcoding reagent binds to the cell membrane of a second cell prior to step (b);
   (b) lysing the cells or permeabilizing the cell membranes of the cells; and
   (c) (separately) annealing or ligating each of the barcoded oligonucleotides of the first multimeric barcoding reagent to at least four sub-sequences of a target nucleic acid of the first cell to produce at least four barcoded target nucleic acid molecules, and (separately) annealing or ligating each of the barcoded oligonucleotides of the second multimeric barcoding reagent to a least four sub-sequences of a target nucleic acid of the second cell to produce at least four barcoded target nucleic acid molecules, wherein the cells are comprised within a single contiguous aqueous volume during steps (a), (b) and (c).
83. The method of clause 82, wherein step (c) comprises:
   (i) annealing each of the barcoded oligonucleotides of the first multimeric barcoding reagent to at least four sub-sequences of a target nucleic acid of the first cell, and annealing each of the barcoded oligonucleotides of the second multimeric barcoding reagent to at least four sub-sequences of a target nucleic acid of the second cell; and
   (ii) extending each of the barcoded oligonucleotides of the first multimeric barcoding reagent to produce at least four different barcoded target nucleic acid molecules and extending each of the barcoded oligonucleotides of the second multimeric barcoding reagent to produce at least four different barcoded target nucleic acid molecules, wherein each of the barcoded target nucleic acid molecules comprises at least one nucleotide synthesised from the target nucleic acid as a template.
84. The method of clause 82 or clause 83, wherein the target nucleic acids are mRNA.
85. A method of synthesising a multimeric barcoding reagent for labelling a target nucleic acid, wherein the method comprises:
   a. synthesizing a library of barcoded oligonucleotides by amplifying a plurality of unique oligonucleotides, wherein each of the plurality of unique oligonucleotides comprises a barcode region and at least one constant region;
   b. contacting the library of barcoded oligonucleotides with at least two multimeric hybridization molecules, wherein each multimeric hybridization molecule is independently linked to a single support and wherein each multimeric hybridization molecule comprises at least two hybridization molecules linked together, wherein each of the hybridization molecules comprises a nucleic acid sequence comprising a hybridization region; and
   c. forming the multimeric barcoding reagent by annealing at least two barcoded oligonucleotides of the library of barcoded oligonucleotides to each of the multimeric hybridization molecules, wherein each barcoded oligonucleotide is annealed to one of the hybridization regions and wherein each barcoded oligonucleotide comprises a barcode region;
   and wherein steps (a), (b) and (c) are performed in a single contiguous aqueous volume.
86. The method of clause 85, wherein each of the plurality of unique oligonucleotides comprises in the 5′ to 3′ direction, a 5′ constant region, a barcode region and a 3′ constant region, and optionally wherein step (a) comprises amplifying each of the plurality of unique oligonucleotides using a pair of primers that anneal to the 5′ constant region and the 3′ constant region.
87. The method of clause 85 or clause 86, wherein the plurality of unique oligonucleotides comprises at least 2, at least 5, at least 10, at least 20, at least 50, at least 100, at least 10³, at least 10⁴, at least 10⁵, at least 10⁶, at least 10⁷ or at least 10⁸ unique oligonucleotides with unique barcode regions.
88. A method of synthesising a library of multimeric barcoding reagent for labelling a target nucleic acid, wherein the method comprises performing in parallel the method of any one of clauses 85-87 in at least two, at least 5, at least 10, at least 100, at least 10³, at least 10⁴, at least 10⁵, at least 10⁶, at least 10⁷ or at least 10⁸ physically separate single contiguous aqueous volumes, optionally wherein each physically separate single contiguous aqueous volume is in a separate well.
89. The method of clause 88, wherein the method further comprises pooling together the physically separate single contiguous aqueous volumes comprising multimeric barcoding reagents to form the library of multimeric barcoding reagents.
90. The method of clause 88 or clause 89, wherein at least 50%, at least 60%, at least 70%, at least 75%, at least 80%, at least 85%, at least 90%, at least 95%, at least 99%, at least 99.9%, at least 99.99%, at least 99.999%, at least 99.9999%, or 100% of the barcode regions of each multimeric barcoding reagent synthesized in each physically separate single contiguous aqueous volume are are different to the barcode regions of the multimeric barcoding reagents formed in the other physically separate single contiguous aqueous volumes.
91. The method of any one of clauses 88-90, wherein the method further comprises sequencing the library of barcoded oligonucleotides in each physically separate single contiguous aqueous volume to generate a profile of the barcode regions of the barcoded oligonucleotides in each physically separate single contiguous aqueous volume, optionally wherein the step of sequencing is performed after step (a) and before step (b).

### BRIEF DESCRIPTION OF THE DRAWINGS

The invention, together with further objects and advantages thereof, may best be understood by making reference to the description taken together with the accompanying drawings, in which:

FIG. 1 illustrates a multimeric barcoding reagent that may be used in the method illustrated in FIG. 3 or FIG. 4.

FIG. 2 illustrates a kit comprising a multimeric barcoding reagent and adapter oligonucleotides for labelling a target nucleic acid.

FIG. 3 illustrates a first method of preparing a nucleic acid sample for sequencing using a multimeric barcoding reagent.

FIG. 4 illustrates a second method of preparing a nucleic acid sample for sequencing using a multimeric barcoding reagent.

FIG. 5 illustrates a method of preparing a nucleic acid sample for sequencing using a multimeric barcoding reagent and adapter oligonucleotides.

FIG. 6 illustrates a method of preparing a nucleic acid sample for sequencing using a multimeric barcoding reagent, adapter oligonucleotides and target oligonucleotides.

FIG. 7 illustrates a method of assembling a multimeric barcode molecule using a rolling circle amplification process.

FIG. 8 illustrates a method of synthesizing multimeric barcoding reagents for labeling a target nucleic acid that may be used in the methods illustrated in FIG. 3, FIG. 4 and/or FIG. 5.

FIG. 9 illustrates an alternative method of synthesizing multimeric barcoding reagents (as illustrated in FIG. 1) for labeling a target nucleic acid that may be used in the method illustrated in FIG. 3 and/or FIG. 4.

FIG. 10 is a graph showing the total number of nucleotides within each barcode sequence.

FIG. 11 is a graph showing the total number of unique barcode molecules in each sequenced multimeric barcode molecule.

FIG. 12 shows representative multimeric barcode molecules that were detected by the analysis script.

FIG. 13 is a graph showing the number of unique barcodes per molecular sequence identifier against the number of molecular sequence identifiers following the barcoding of synthetic DNA templates of known sequence with multimeric barcoding reagents containing barcoded oligonucleotides.

FIG. 14 is a graph showing the number of unique barcodes per molecular sequence identifier against the number of molecular sequence identifiers following the barcoding of synthetic DNA templates of known sequence with multimeric barcoding reagents and separate adapter oligonucleotides.

FIG. 15 is a table showing the results of barcoding genomic DNA loci of three human genes (BRCA1, HLA-A and DQB1) with multimeric barcoding reagents containing barcoded oligonucleotides.

FIG. 16 is a schematic illustration of a sequence read obtained from barcoding genomic DNA loci with multimeric barcoding reagents containing barcoded oligonucleotides.

FIG. 17 is a graph showing the number of barcodes from the same multimeric barcoding reagent that labelled sequences on the same synthetic template molecule against the number of synthetic template molecules.

FIG. 18 illustrates examples of multimeric barcoding reagents comprising cell-binding moieties.

FIG. 19 illustrates a method of transferring multimeric barcoding reagents into cells via cell-binding moieties.

FIG. 20 illustrates a method of transferring multimeric barcoding reagents into cells via liposomal delivery.

FIG. 21 illustrates a method of transferring multimeric barcoding reagents into cells via transfection.

FIG. 22 illustrates a method of transferring multimeric barcoding reagents into cells via a permeabilisation process.

FIG. 23 illustrates a method of barcoding cellular nucleic acids with a membrane-permeabilisation step.

FIG. 24 illustrates a method of barcoding cellular nucleic acids with a membrane-permeabilisation and barcoded oligonucleotide-release step.

FIG. 25 illustrates options for the attachment of barcoded oligonucleotides to a support.

FIG. 26 illustrates options for annealing barcoded oligonucleotides to a multimeric hybridization molecule.

FIG. 27 illustrates options for linking a cell-binding moiety to a barcoded oligonucleotide or a multimeric hybridization molecule.

FIG. 28 is a graph showing barcoded oligonucleotides per bead with different types of beads having also different sizes.

FIG. 29 is a graph showing effect of optimization of coupling conditions on 32.2 μm beads. As visible, by optimising coupling conditions it is possible to increase/vary the number of barcoded oligonucleotides that a single bead-based multimeric barcoding reagent can carry.

FIG. 30 is a graph showing the effect of adding a second segment of multimeric hybridization molecule via CuAAC coupling on the number of barcoded oligonucleotides per bead. As visible, adding a second multimeric hybridization molecule segment increases the number of barcoded oligonucleotides that a single bead-based multimeric barcoding reagent can carry.

FIG. 31 shows two graphs showing the amount of barcoded oligonucleotides carried by different multimeric hybridization molecules attached to 14.5 μm beads as a support. It is possible to see how different multimeric hybridization molecules can either maintain similar amounts of barcoded oligonucleotides or they can instead show different loadings. Error bars represent the standard error of the mean.

FIG. 32 is a graph showing that adjusting the concentration (v/v) of 4 k-15 k Poly-L-Lysine during a reagent ‘activation’ step impacts the cell-binding capability of treated reagents. Log scale shown.

FIG. 33 shows graphs showing the results of cell binding experiments with variable binding conditions.

FIG. 34 is a graph showing that in a 30 μm bead settling experiment.

FIG. 35 is a graph showing the effect of adding a cell binding moiety to the % of cells bound by a bead based multimeric barcoding reagent.

FIG. 36 illustrates the schematics of cell-multimeric barcoding reagent binding methodologies. A. Bead-based cell-multimeric barcoding reagents are settled to the bottom of the tube and then cells are settled/layered on top of this layer to induce binding events on a 2D plane. Samples are subsequentially disrupted to ensure all components are in solution before proceeding to next steps. B. Cells are settled to the bottom of the tube and then bead-based cell-multimeric barcoding reagents are settled/layered on top of this layer to induce binding events on a 2D plane. Samples are subsequentially disrupted to ensure all components are in solution before proceeding to next steps. C. Cell and bead-based multimeric barcoding reagents are mixed in solution to allow binding events to occur. This method can consist of mixing samples together or utilizing a rotation method over a period to encourage further cell binding events to occur.

FIG. 37 is a graph showing that cell binding studies identified that when beads were settled in tube first (20 minutes beads settling then 20 minutes cell settling), a higher observed reagent-cell binding was observed in comparison to the the opposite arrangement in which cells were settled first and beads settled on top. Mean and standard deviations shown.

FIG. 38 shows schematics and optical microscope images of bead-format multimeric barcoding reagents binding to cells. Solid-phase beads are used as the support for the multimeric hybridization molecules and hence the barcoded oligonucleotides. A. Multimeric barcoding reagents bind to cells via cell-binding moieties. A potential cell binding moiety may function via membrane insertion. B. Three examples of single cell with multimeric barcoding reagents binding. One cell and one multimeric barcoding reagent are unbound. C. Two cell-multimeric barcoding reagent binding events.

FIG. 39 is a schematic showing that following cell binding to multimeric barcoding reagents and dilution for the purposes of kinetic exclusion, cell membranes are permeabilized leading to target molecule release from cells. The viscosity agent limits kinetic diffusion of barcoded oligonucleotides and target molecules of interest to prevent cross-talk. In this example, mRNA anneals to barcoded oligonucleotides via the affinity sequence.

FIG. 40 is a schematic of barcoded oligonucleotide extension. In this example, mRNA is the target molecule, however the principle of barcoded oligonucleotide extension applied to other target molecules. Barcoded oligonucleotide extension allows the barcoding of the target molecule.

FIG. 41 is a schematic showing that following barcoded oligonucleotide extension, a random-priming and extension method is utilized for the purposes of sample length diversity generation and introducing a sequencing adaptor compatible oligo to the barcoded molecule of interest. In this example, the product of mRNA barcoding is random-primed and extended.

FIG. 42 is a high-level assay workflow schematic. Following cell-reagent binding and barcoding of target nucleic acids, samples are captured, and the barcoded oligonucleotides are extended. Random priming and extension is then performed prior to PCR amplification reactions for the purposes of library amplification and introduction of sequencing adaptor sequences. Sequencing is then performed and bioinformatic analysis identifies single-cells via sample barcode identification.

FIG. 43 is a sequencing workflow schematic and % base profile during sequencing examples. A. During the first read of sequencing a short oligonucleotide region which was used in the random priming event is covered and then the read proceeds into the application specific content region. In the second and third reads of sequencing the sequencing platform index reads are performed, which can be used to de-multiplex the sample following sequencing. In the fourth read, the barcode region of the sample is covered, an anti-slip region may also be covered. B. Sequencing % base profiles for a single cell mRNA capture library. A barcoded oligonucleotide with a 6N barcode sequence and a single anti-slip base was used in this assay. C. Sequencing % base profiles for a single cell mRNA capture library. A barcoded oligonucleotide with a 16N barcode sequence and a five base anti-slip region was used in this assay.

FIG. 44 shows: A. Single species de-duplicated uniquely mapped reads for human (Hsap; Homo Sapiens) or mouse (Mmus; Mus Musculus) only control samples; and B. Human/Mouse species mixing results.

FIG. 45 shows human cell genomic coverage of a successful single cell mRNA capture.

FIG. 46 is a schematic of barcoded oligonucleotide dilution principle. From a pool of high barcode diversity, represented by various shapes and colors, dilutions can be prepared to reduce the barcode diversity within resulting aliquots. Such resulting pools can be used as the ‘seed’ oligonucleotides for diversity generation PCR steps.

FIG. 47 shows graphs of barcoded oligonucleotide diversity assessment after the preparation of two multimeric barcoding reagent library pools. In order to register during analysis, each oligo required at least two reads of coverage observed during decoding sequencing. A. A library consisting of 384 multimeric barcoding reagents was prepared to contain alternating molecular concentrations of input barcoded oligonucleotides; a clear division in total unique barcoded oligonucleotides was observed across the library. B. A library consisting of 3,072 multimeric barcoding reagents was prepared to contain an input molecule amount of 500-1,000 seed molecules. With the exception of several sample drop-outs, a consistent number of total unique barcoded oligonucleotides was observed across the library.

FIG. 48 provides simplified schematics of barcoded oligonucleotide amplification methodologies. A. Possible oligonucleotide structure of barcoded oligonucleotide molecules. B. Diversity generation PCR principle. Primers used in this reaction use the sequencing adaptor sequence as one annealing site, the companion primer uses the target affinity sequence, and, if required, an anti-slip sequence in order to prevent slippage if the target affinity sequence consists of a homo-polymer sequences or a repeating sequence.

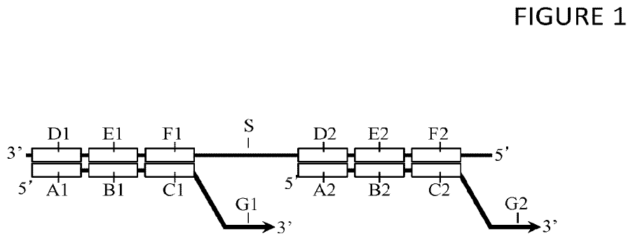

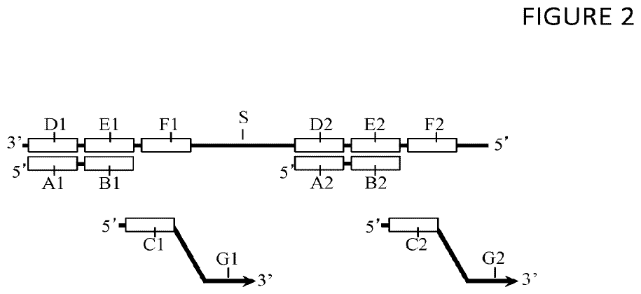

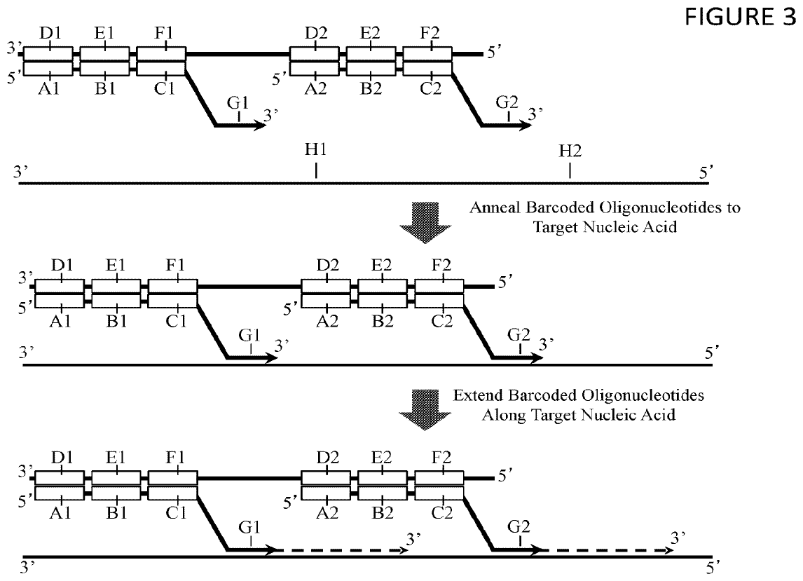

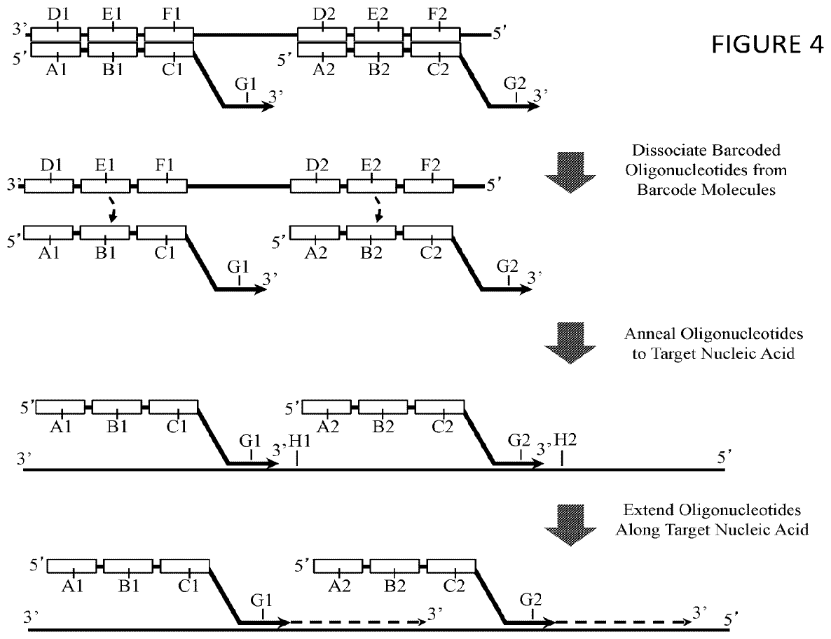

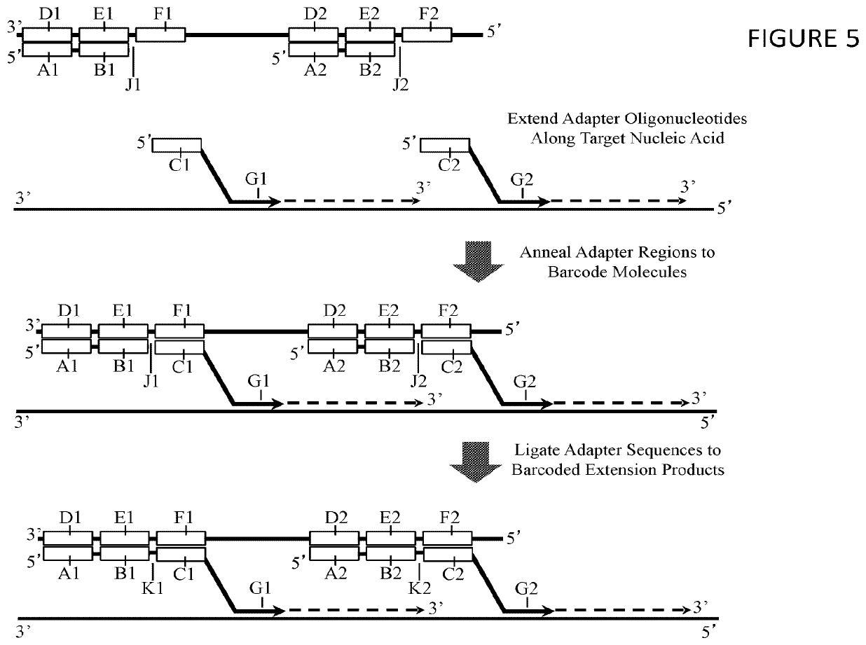

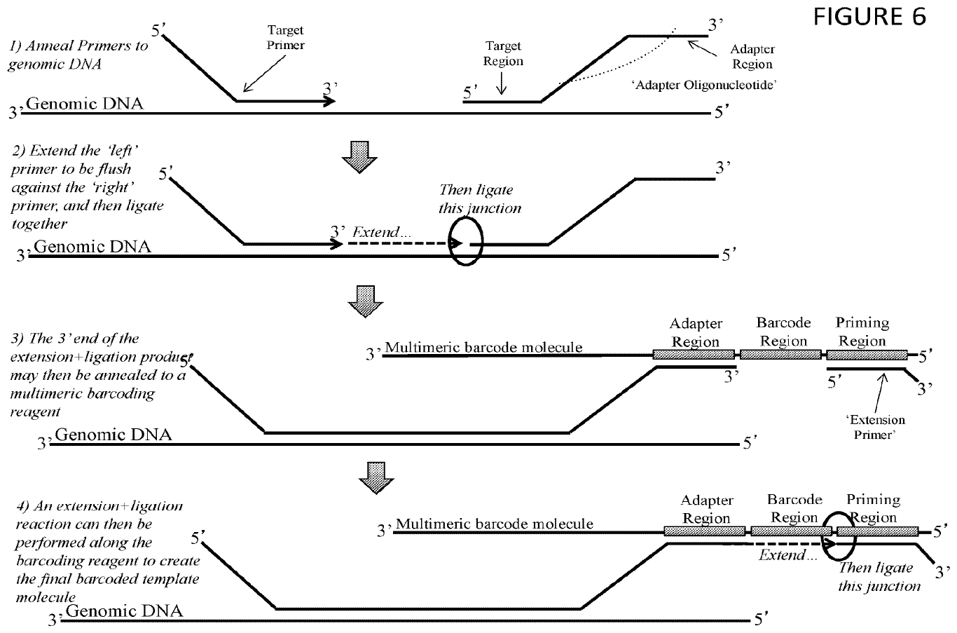

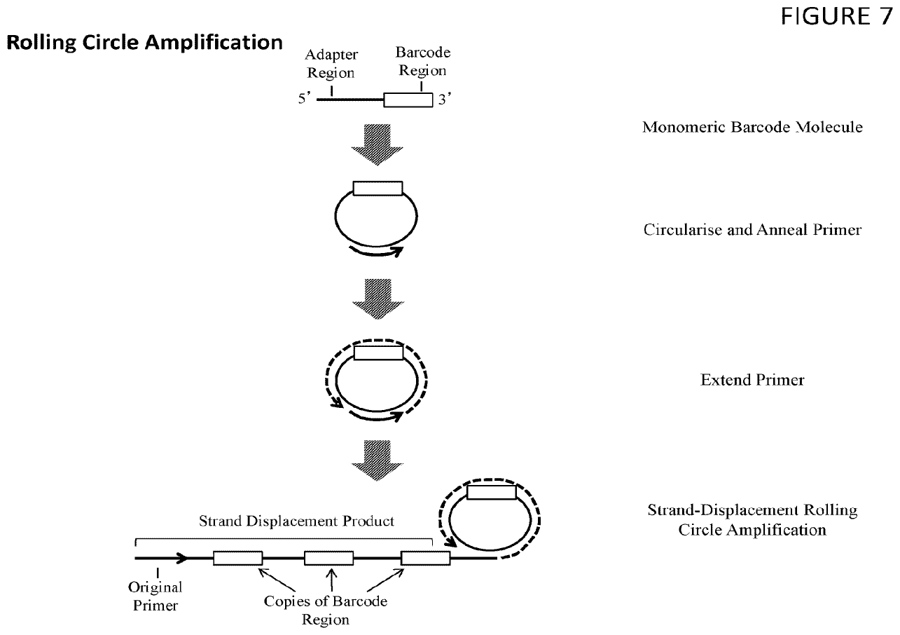

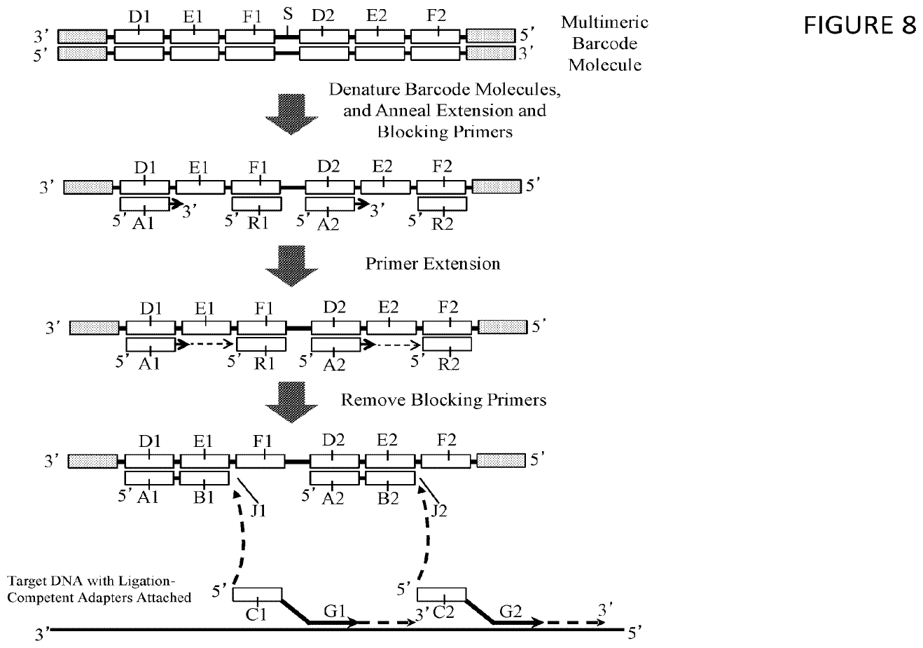

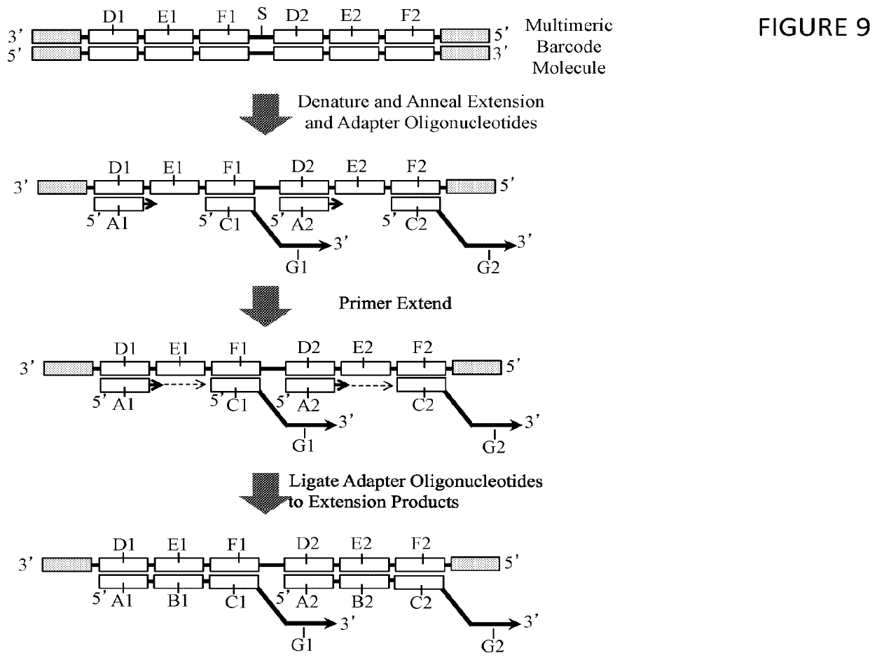

FIG. 49 is a simplified schematic of highly adaptable barcoded oligonucleotide generation process.
A. A randomer barcode oligonucleotide sequence flanked by adaptor sites can be used as the seed molecule for additional amplification. A pool of high barcode diversity can be serially diluted to barcode diversity input numbers required and then amplified with primers designed for adaptor site complementarity. This amplifies a defined barcode diversity within each well to a yield usable in following steps.
B. Amplified barcode oligonucleotide with adaptor sites can be used as a template for further amplification with a variety of 5′ overhanging primers which share complementarity to the adaptor sites of the oligonucleotide at their 3′ ends. This allows flexible selection capabilities of target affinity oligonucleotide sequences, sequencing platform adaptor sequences and multimeric hybridization sequence from common barcode diversity generation reaction. This interchangeability of terminal oligonucleotide sequences allows for great flexibility in application selection, sequencing platform selection and multimeric hybridization molecule selection from common barcode diversity generation reaction. This adaptability may also allow for co-production of multiple types of affinity sequence within a single reaction. This ability to re-use a common barcode diversity generation reaction also reduces requirements for multiple decoding sequencing events.

FIG. 50 illustrates methods for the single stranded selection of the barcoded oligonucleotide. A. Amplification can be performed with a blocked primer for the undesired candidate strand. B. Selectively 5′ phosphorylated primers can be used to allow for selective exonuclease digestion of undesired strands. C. A single primer is used in excess in order to bias the product towards the desired strand.

FIG. 51 provides multimeric hybridization molecule schematics. A. Without sufficient viscosity agent present within the hybridization buffer, bead-based reagent scaffolds settle in solution if rotation is not utilized. B. Following hybridization to multimeric hybridization molecules, free barcoded oligonucleotides may be quenched utilizing hybridization block oligonucleotides. C. Potential annealing positioning of barcoded oligonucleotides to a multimeric hybridization molecule, which is attached to a bead-based scaffold.

FIG. 52 is a graph showing the results of trialing various viscosity agents and additives across different hybridization timeframes.

FIG. 53 shows graphs showing that hybridization blocking oligonucleotides can reduce bead binding without displacing annealed barcoded oligonucleotides in the absence of 2M NaCl in the quenching buffer. A. In a hybridization-prevention assay, unhybridized multimeric hybridization molecules were exposed to barcoded oligonucleotides in the context of a quenching buffer containing various lengths of hybridization blocking oligonucleotides and various buffer backgrounds. In all conditions, a hybridization blocking oligonucleotide reduced hybridization effectiveness. B. To determine if hybridization blocking oligonucleotides were de-hybridizing barcoded oligonucleotides from the multimeric hybridization molecules a quenching assay was performed. This identified that increasing the NaCl concentration reduced the number of molecules per bead following treatment, however the hybridization blocking oligonucleotides alone did not reduce this in comparison to a PBS background. None-Water. Mean and standard deviation shown.

FIG. 54 is a schematic of barcoded oligonucleotide related methods. A. Possible oligonucleotide structure of decoding template oligonucleotide molecules. B. Barcoded oligonucleotides can be extended with a decoding template oligonucleotide to increase length and allow for an indexed sequencing adaptor annealing. Indexed sequencing adaptors may then be PCR amplified onto the product.

FIG. 55 is a decoding workflow schematic and % base profile during sequencing. During the first read of sequencing at least 26 bases of the N-mer region are covered, this is for purposes of sequencing registration. In the second and third reads of sequencing the sequencing platform index reads are performed, which can be used to de-multiplex the sample following sequencing. In the fourth read, the barcode region of the sample is covered, an anti-slip region may also be covered. Together, reads 2, 3 and 4 can allow determination of barcoded oligonucleotide diversity within each sample of a multi-reagent library pool. In % base profile figure, a 16N barcoded oligonucleotide with five anti-slip bases was used.

FIG. 56 provides graphs that show single-cell sequence of messenger RNA transcript from a sample comprising a mixture of human cells and mouse cells. Shown are raw and unique (deduplicated) read counts for sequences mapping to the human genome (Hsap) and mouse genome (Mmus) along the vertical and horizontal axes respectively, with each individual dot representing reads derived from an individual multimeric barcoding reagent within the library of multimeric barcoding reagents. Reagents exhibiting a larger number of reads and located along the vertical or horizontal axes represent reagents bound to, and then successfully used to barcode, target nucleic acids (i.e. messenger RNA molecules) from single human or mouse cells respectively.

FIG. 57 illustrates the number of single-cells identified in the context of freeze-thaw versus without freeze-thaw.

FIG. 58 shows the number of Homo Sapiens or Mus Musculus genes identified per cell in the context of freeze-thaw versus without freeze-thaw.

FIG. 59A&B illustrates Human/Mouse species mixing results with or without freeze-thaw in lysis buffer. FIG. 59 C&D shows the total nuclear gene count from each single-cell.

FIG. 60 shows the number of single-cells identified per sample. Increasing formamide concentration in the lysis buffer increases the number of cells captured.

FIG. 61A&B illustrates Human/Mouse species mixing results with or without additive in lysis buffer. FIG. 61 C&D shows the total nuclear gene count from each single-cell.

FIG. 62A&B illustrates Human/Mouse species mixing results with or without additive in lysis buffer.

FIG. 63A illustrates cell output results for one embodiment of the method of preparing a nucleic acid sample for sequencing in which the plasticware was coated with 0.1% BSA and MgCl2 was added to a sequencing step. FIG. 63 B illustrates cell output results for one embodiment of the method of preparing a nucleic acid sample for sequencing, in which the plasticware was coated with 0.1% BSA and the step of contacting the sample with the library was performed using gentle centrifugation.

FIG. 64A illustrates transcript and gene number results for one embodiment of the method of preparing a nucleic acid sample for sequencing in which the plasticware was coated with 0.1% BSA and different post-capture wash buffers and volumes were analysed. FIG. 64 B illustrates nuclear gene number results for one embodiment of the method of preparing a nucleic acid sample for sequencing.

FIGS. 65A and B shows deep sequencing results for two embodiments of the method of preparing a nucleic acid sample for sequencing.

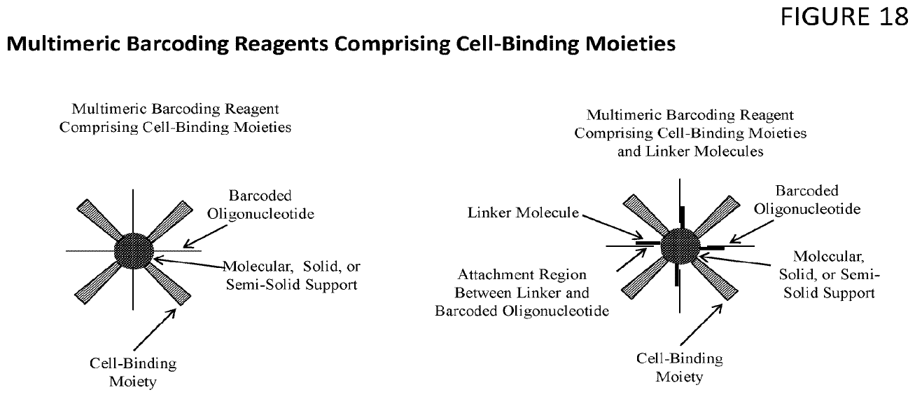

FIG. 18 illustrates examples of multimeric barcoding reagents comprising cell-binding moieties. The figure shows two different schematic variants of a multimeric barcoding reagent comprising cell-binding moieties. In a first such embodiment (left), a number of cell-binding moieties are attached to a support (such as a bead, or a nucleic acid molecule), and a number of barcoded oligonucleotides are likewise attached to the support. The cell-binding moieties may comprise any sort of molecule or compound able to preferentially interact with cell surfaces, such as antibodies or aptamers which have affinity for specific proteins on the surface of cells, or charge molecules such as poly-lysine moieties which have electrostatic affinity for the charged cell membrane. The attachment of such cell-binding moieties and barcoded oligonucleotides to the support may be direct (e.g. through direct covalent chemical complexation), may be non-covalent (e.g. through protein-protein interactions), and/or may be indirect, such as involving secondary attachment molecules.

In a second embodiment (on right), a number of cell-binding moieties are appended to a support, as are a number of linker molecules comprising a nucleic acid sequence. These linker molecules may be attached directly to the support (e.g. through chemical complexation), or through any other indirect and/or non-covalent binding. A barcoded oligonucleotide is annealed to the nucleic acid sequence of each linker molecule, thus forming an indirect attachment of each barcoded oligonucleotide to the support within the overall multimeric barcoding reagent. The hybridization region formed between the linker molecules and the barcoded oligonucleotides may further allow for manipulation of the interaction between the barcoded oligonucleotides and the support; for example, a high temperature incubation process may be used to denature the hybridization region and thus allow barcoded oligonucleotides to diffuse away in solution from the support itself.

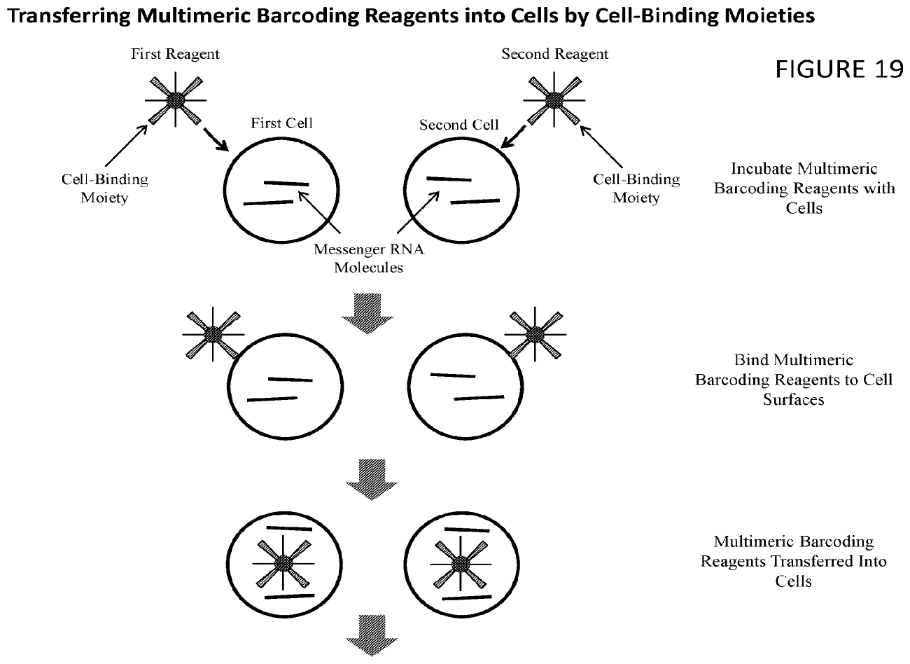

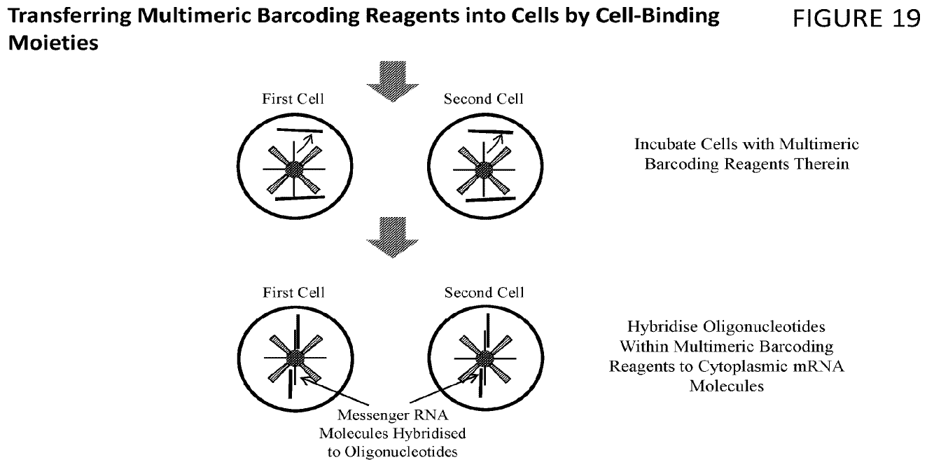

FIG. 19 illustrates an example of a method of transferring multimeric barcoding reagents into cells via cell-binding moieties. In the method, multimeric barcoding reagents are transferred into cells by a transfer process involving cell-binding moieties. These cell-binding moieties may comprise any sort of molecular, macromolecular, and/or solid moiety that is capable of preferentially interacting with a cell. For example, this may comprise an antibody capable of binding to a specific protein on the cell surface; alternatively, for example, this may comprise a cationic macromolecule such as a poly-lysine moiety that preferentially interacts with the cell surface by electrostatic attraction.

In a first step, a library of two or more multimeric barcoding reagents each comprising one or more cell-binding moieties are incubated with a sample of cells for a period of time, during which time the multimeric barcoding reagents migrate to come into contact with a cell membrane, and become bound to said cell membrane via one or more associated cell-binding moieties.

In a second step following this cell-binding step, the sample of cells bound to multimeric barcoding reagents is incubated for a period of time, during which time multimeric barcoding reagents are transferred into cells. This transfer process may be effected by any one or more known process of cells internalising constituents bound to or within their cell membrane, such as endocytosis, pinocytosis, and/or phagocytosis. In this illustration, a first multimeric barcoding reagent-lipid complex is transferred into a first cell, and a second multimeric barcoding reagent-lipid complex is transferred into a second cell; in actual embodiments a large library of multimeric barcoding reagents may be transferred into a large sample of cells.

Following this transfer step, an incubation step is performed, during which time messenger RNA molecules complementary to the target regions of barcoded oligonucleotides comprised within the transferred multimeric barcoding reagents are allowed to anneal to said target regions. This incubation may be performed at a temperature conducive to such an annealing process, and/or may be performed in the presence of a modified annealing buffer which may be conducive to such an annealing process (such as a buffer containing a nucleic acid denaturant, such as betaine or DMSO).

Following the annealing step, messenger RNA molecules from individual cells are thus annealed to barcoded oligonucleotides from the multimeric barcoding reagent which was transferred into that cell. In subsequent processing steps (for example, after a step of isolating the annealed messenger RNA molecules and barcoded oligonucleotides), the messenger RNA may be reverse-transcribed with a reverse transcriptase, and then optionally amplified such as with a PCR process, prior to performing a sequencing reaction. The reverse transcription may include either and/or both first-strand reverse transcription (e.g. first-strand cDNA synthesis) and also second-strand synthesis. Furthermore, any step of reverse transcription and/or cDNA synthesis may include any further standard step of cDNA processing, such as fragmentation (e.g. acoustic fragmentation such as Covaris sonication, or e.g. enzymatic fragmentation such as with a fragmentase enzyme, a restriction enzyme, and/or an in vitro transposase enzyme) and adapter (e.g. PCR adapter and/or sequencing adapter) ligation and/or adapter in vitro transposition at any stage(s) prior to and/or after reverse transcription and/or second strand synthesis and/or PCR.

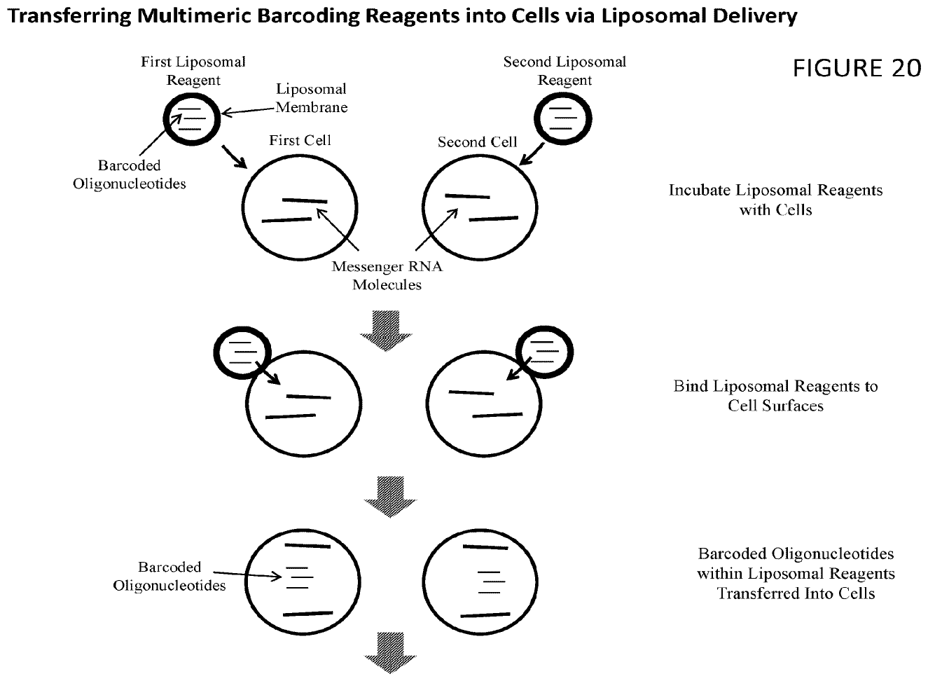

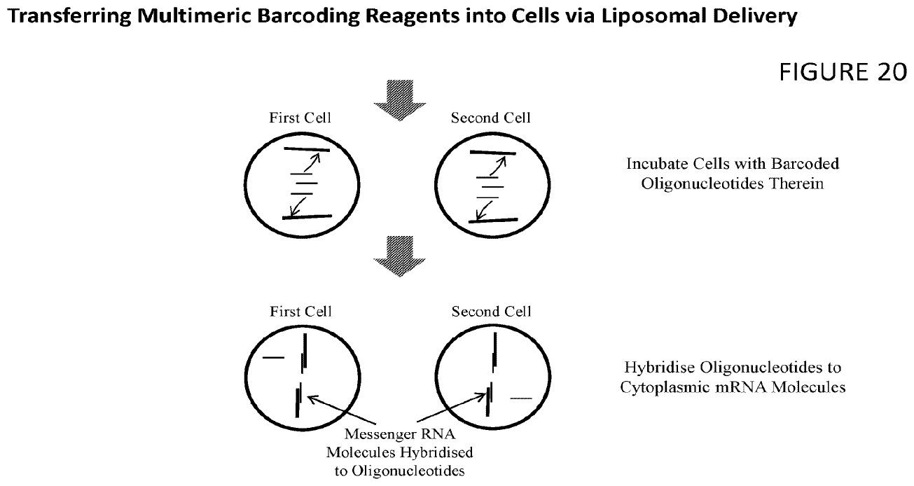

FIG. 20 illustrates a method of transferring multimeric barcoding reagents into cells via liposomal delivery. In the method, multimeric barcoding reagents are transferred into cells by a transfer process involving barcoded oligonucleotides being comprised within liposomal compounds, and then transferring said barcoded oligonucleotides by liposomal delivery. In this embodiment, barcoded oligonucleotides are encapsulated within liposomes. These barcoded oligonucleotides may optionally be associated with other molecular moieties.

In a first step, the library of liposomes is incubated with a sample of two or more cells, and the liposomes are allowed to interact with the cell membranes of cells within the sample. As with standard liposomal delivery methods, the liposome may then fuse with the cell membrane, and/or be internalised into the cell, and release its constituent barcoded oligonucleotides into the cytoplasm, thus achieving liposomal delivery of barcoded oligonucleotides into cells of the sample.

Following this liposomal-delivery step, an incubation step is performed, during which time messenger RNA molecules complementary to the target regions of barcoded oligonucleotides delivered by the liposomes are allowed to anneal to said target regions. This incubation may be performed at a temperature conducive to such an annealing process, and/or may be performed in the presence of a modified annealing buffer which may be conducive to such an annealing process (such as a buffer containing a nucleic acid denaturant, such as betaine or DMSO).

Following the annealing step, messenger RNA molecules from individual cells are thus annealed to barcoded oligonucleotides that have been delivered by a liposome. In subsequent processing steps (for example, after a step of isolating the annealed messenger RNA molecules and barcoded oligonucleotides), the messenger RNA may be reverse-transcribed with a reverse transcriptase, and then optionally amplified such as with a PCR process, prior to performing a sequencing reaction.

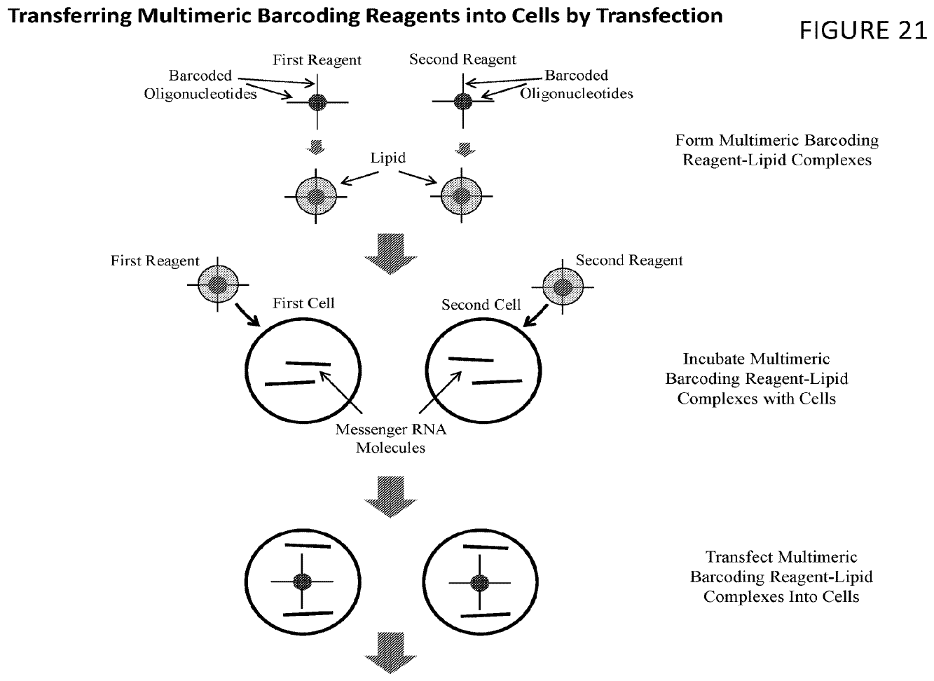

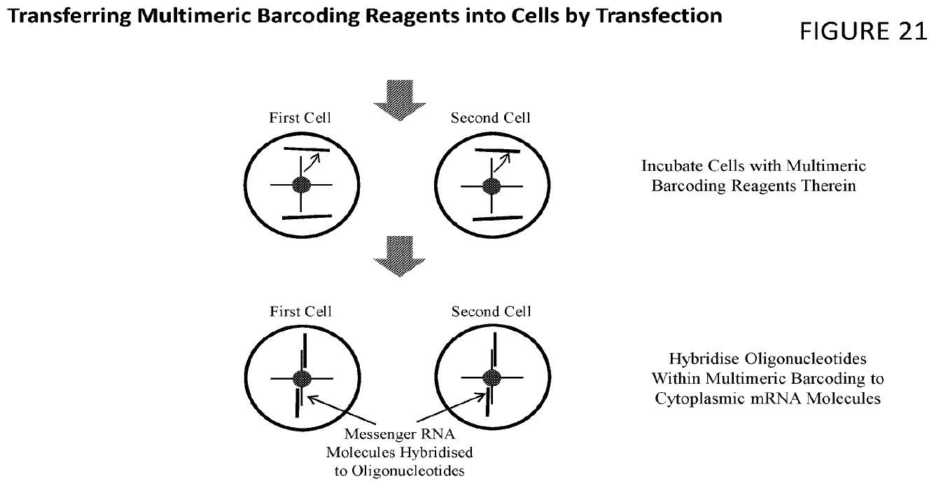

FIG. 21 illustrates an example of a method of transferring multimeric barcoding reagents into cells via transfection. In the method, multimeric barcoding reagents are transferred into cells by a transfection process. In a first step, multimeric barcoding reagents (e.g, barcoded oligonucleotides annealed along a multimeric barcode molecule) are complexed with a lipid transfection reagent. These complexes, analogous to lipid-complexed plasmids, will have biophysical and electrostatic character conducive to interaction with a cell membrane and then transfection into cells.

The resulting multimeric barcoding reagent-lipid complexes are then incubated with a sample of cells for a period of time, during which time the complexes migrate to come into contact with a cell membrane, and are transfected into cells. In this illustration, a first multimeric barcoding reagent-lipid complex is transfected into a first cell, and a second multimeric barcoding reagent-lipid complex is transfected into a second cell; in actual embodiments a large library of multimeric barcoding reagents may be transfected into a large sample of cells.

Following this transfection step, an incubation step is performed, during which time messenger RNA molecules complementary to the target regions of barcoded oligonucleotides comprised within the transfected multimeric barcoding reagents are allowed to anneal to said target regions. This incubation may be performed at a temperature conducive to such an annealing process, and/or may be performed in the presence of a modified annealing buffer which may be conducive to such an annealing process (such as a buffer containing a nucleic acid denaturant, such as betaine or DMSO).

Following the annealing step, messenger RNA molecules from individual cells are thus annealed to barcoded oligonucleotides from the multimeric barcoding reagent which was transferred into that cell. In subsequent processing steps (for example, after a step of isolating the annealed messenger RNA molecules and barcoded oligonucleotides), the messenger RNA may be reverse-transcribed with a reverse transcriptase, and then optionally amplified such as with a PCR process, prior to performing a sequencing reaction

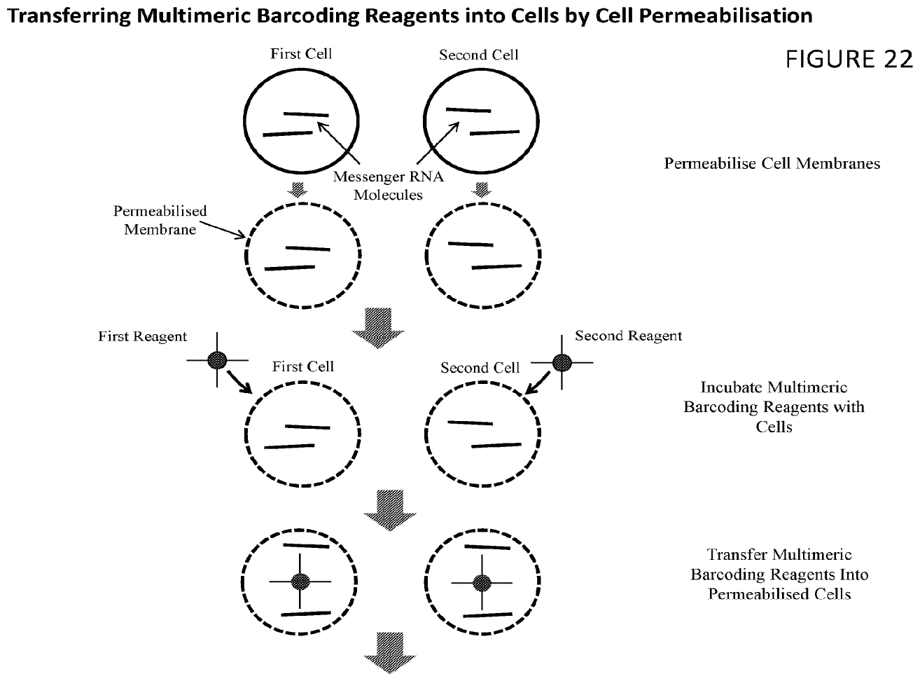

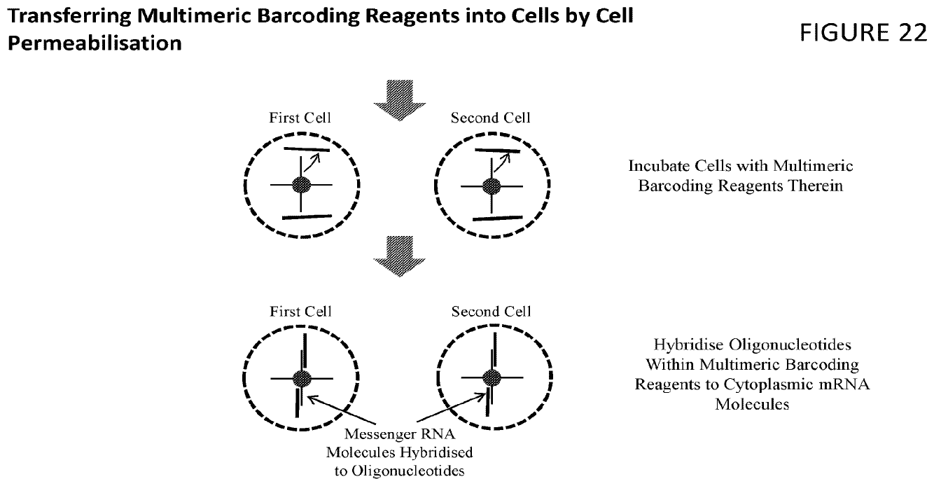

FIG. 22 illustrates an example of a method of transferring multimeric barcoding reagents into cells via a permeabilisation process. In the method, multimeric barcoding reagents are transferred into cells by a permeabilisation process. In a first step, the membranes of cells are permeabilised with a permeabilisation process. This may, in one embodiment, be performed by exposure to a chemical surfactant such as a non-ionic detergent. Following this permeabilisation process, the membrane of each cell will have biophysical character conducive to diffusion of macromolecular species such as multimeric barcoding reagents therethrough.

The resulting permeabilised cells are then incubated with a library of two or more multimeric barcoding reagents for a period of time, during which time the multimeric barcoding reagents migrate to come into contact with a cell membrane, and are transferred into cells by a diffusion process. In this illustration, a first multimeric barcoding reagent diffuses into a first cell, and a second multimeric barcoding reagent diffuses into a second cell; in actual embodiments a large library of multimeric barcoding reagents may be transferred into a large sample of cells by this method.

Following this diffusion step, an incubation step is performed, during which time messenger RNA molecules complementary to the target regions of barcoded oligonucleotides comprised within the transferred multimeric barcoding reagents are allowed to anneal to said target regions. This incubation may be performed at a temperature conducive to such an annealing process, and/or may be performed in the presence of a modified annealing buffer which may be conducive to such an annealing process (such as a buffer containing a nucleic acid denaturant, such as betaine or DMSO).

Following the annealing step, messenger RNA molecules from individual cells are thus annealed to barcoded oligonucleotides from the multimeric barcoding reagent which was transferred into that cell. In subsequent processing steps (for example, after a step of isolating the annealed messenger RNA molecules and barcoded oligonucleotides), the messenger RNA may be reverse-transcribed with a reverse transcriptase, and then optionally amplified such as with a PCR process, prior to performing a sequencing reaction.

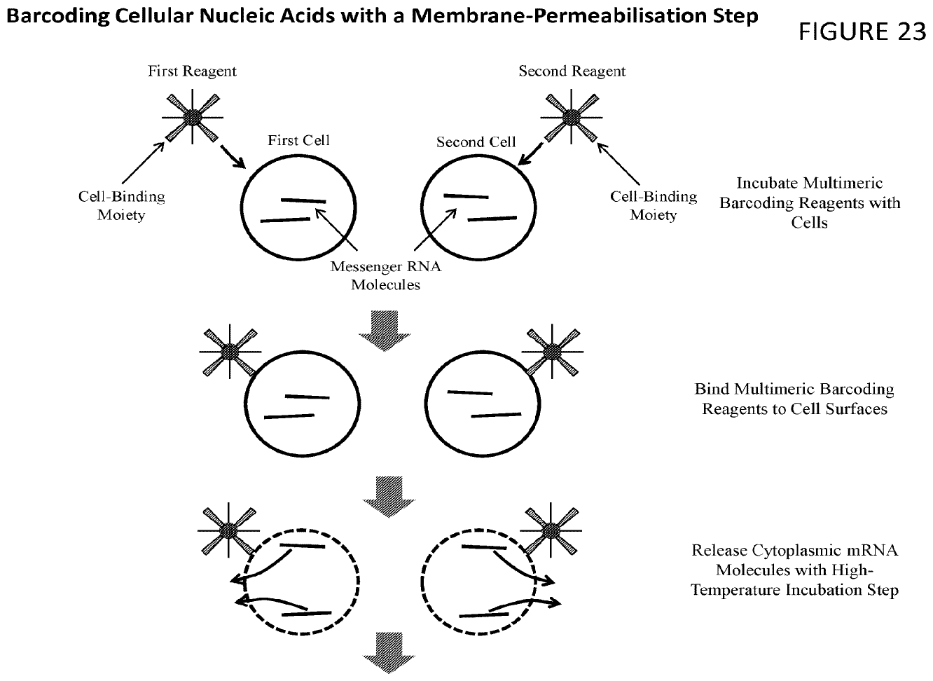

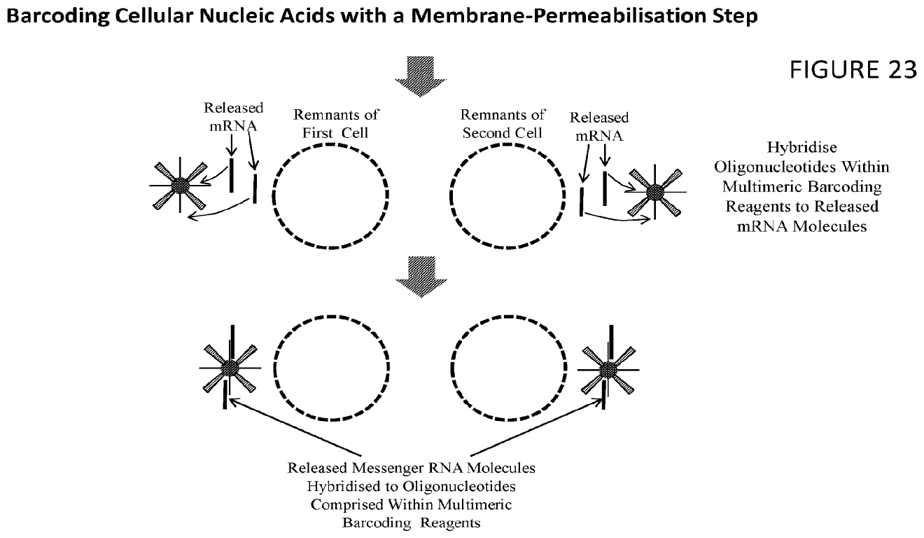

FIG. 23 illustrates an examples of a method of barcoding cellular nucleic acids with a membrane-permeabilisation step. In the method, messenger RNA molecules are released from cells, whereupon they are barcoded by barcoded oligonucleotides that are within spatial proximity of the cell itself. In a first step, a library of two or more multimeric barcoding reagents are mixed with a sample of two or more cells. Optionally, as shown, said multimeric barcoding reagents may comprise cell-binding moieties which drive them to preferentially interact with the membranes of cells within the samples; an incubation step is performed to allow the multimeric barcoding reagents to bind to the cell surfaces.

In a second step, a membrane-permeabilisation and/or cell lysis process is performed, in which the cell membrane is made permeable to macromolecules such that messenger RNA molecules and/or oligonucleotides may diffuse through the membrane space. This step may be performed by a number of means, such as by a high-temperature incubation step as illustrated here. This permeabilisation and/or lysis step enables molecular interaction between barcoded oligonucleotides and their target nucleic acids.

Following this membrane-permeabilisation and/or lysis step, an incubation step is performed, during which time messenger RNA molecules complementary to the target regions of barcoded oligonucleotides comprised within the multimeric barcoding reagents are allowed to anneal to said target regions. This incubation may be performed at a temperature conducive to such an annealing process, and/or may be performed in the presence of a modified annealing buffer which may be conducive to such an annealing process (such as a buffer containing a nucleic acid denaturant, such as betaine or DMSO). This incubation may further be performed in the presence of a thickening agent, such as poly(ethylene) glycol (PEG), to retard the diffusion of barcoded oligonucleotides and/or target nucleic acid molecules within solution.

Following the annealing step, messenger RNA molecules from individual cells are thus annealed to barcoded oligonucleotides from the multimeric barcoding reagent which was within spatial proximity to that cell. In subsequent processing steps (for example, after a step of isolating the annealed messenger RNA molecules and barcoded oligonucleotides), the messenger RNA may be reverse-transcribed with a reverse transcriptase, and then optionally amplified such as with a PCR process, prior to performing a sequencing reaction.

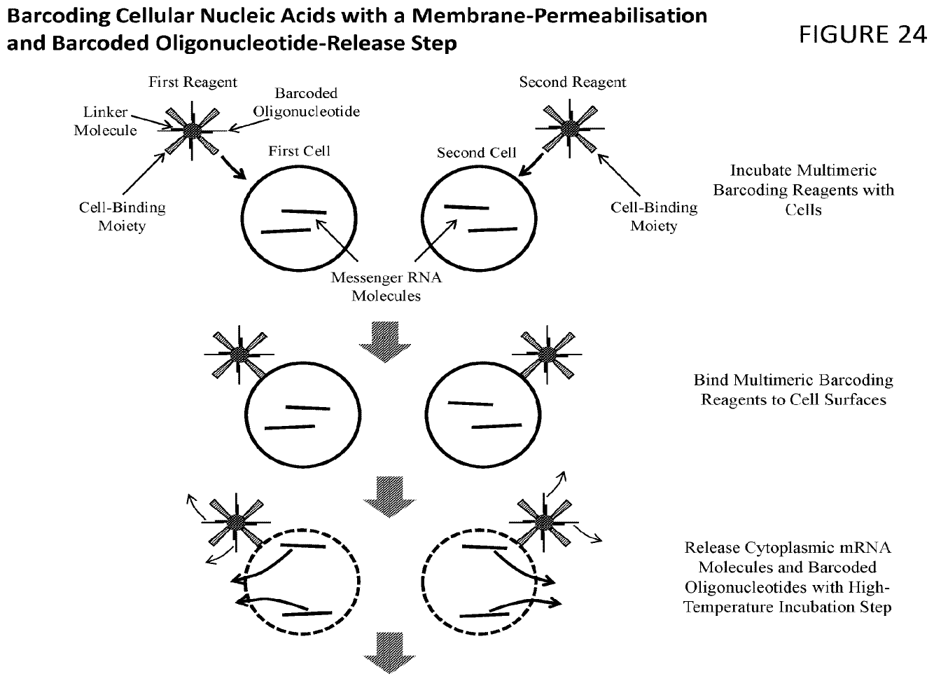

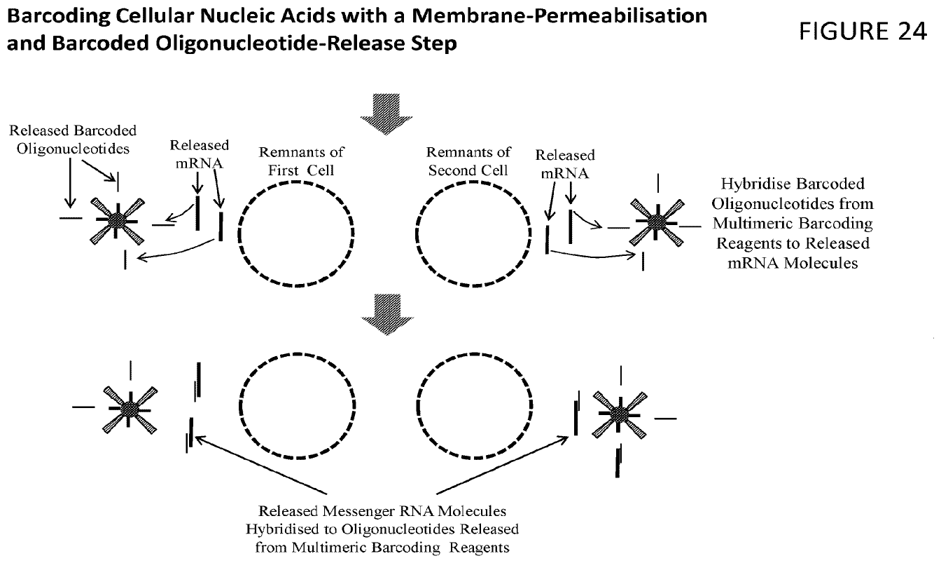

FIG. 24 illustrates a method of barcoding cellular nucleic acids with a membrane-permeabilisation and barcoded oligonucleotide-release step. In the method, messenger RNA molecules may be released from cells, whereupon they are barcoded by barcoded oligonucleotides that are released from multimeric barcoding reagents that were within spatial proximity to the cell said itself. In a first step, a library of two or more multimeric barcoding reagents are mixed with a sample of two or more cells. Optionally, as shown, said multimeric barcoding reagents may comprise cell-binding moieties which drive them to preferentially interact with the membranes of cells within the samples; an incubation step is performed to allow the multimeric barcoding reagents to bind to the cell surfaces.

In a second step, a membrane-permeabilisation and/or cell lysis process is performed, in which the cell membrane is made permeable to macromolecules such that messenger RNA molecules and/or oligonucleotides may diffuse through the membrane space. This step may be performed by a number of means, such as by a high-temperature incubation step as illustrated here. This permeabilisation and/or lysis step enables molecular interaction between barcoded oligonucleotides and their nucleic acid targets.

In this embodiment, this high-temperature incubation step further dissociates barcoded oligonucleotides from their respective multimeric barcoding reagents-specifically in this embodiment, said barcoded oligonucleotides are annealed to linker molecules which themselves are appended to the solid/molecular support of each multimeric barcoding reagent. This high-temperature incubation step is performed at a temperature above the melting temperature of the barcoded oligonucleotide-linker hybridization region, and thus the barcoded oligonucleotides become free to diffuse in solution.

Following this membrane-permeabilisation and/or lysis step, an incubation step is performed, during which time messenger RNA molecules complementary to the target regions of barcoded oligonucleotides released from the multimeric barcoding reagents are allowed to anneal to said target regions. This incubation may be performed at a temperature conducive to such an annealing process, and/or may be performed in the presence of a modified annealing buffer which may be conducive to such an annealing process (such as a buffer containing a nucleic acid denaturant, such as betaine or DMSO). This incubation may further be performed in the presence of a thickening agent, such as poly(ethylene) glycol (PEG), to retard the diffusion of barcoded oligonucleotides and/or target nucleic acid molecules within solution.

Following the annealing step, messenger RNA molecules from individual cells are thus annealed to barcoded oligonucleotides released from the multimeric barcoding reagent which was within spatial proximity to that cell. In subsequent processing steps (for example, after a step of isolating the annealed messenger RNA molecules and barcoded oligonucleotides), the messenger RNA may be reverse-transcribed with a reverse transcriptase, and then optionally amplified such as with a PCR process, prior to performing a sequencing reaction.

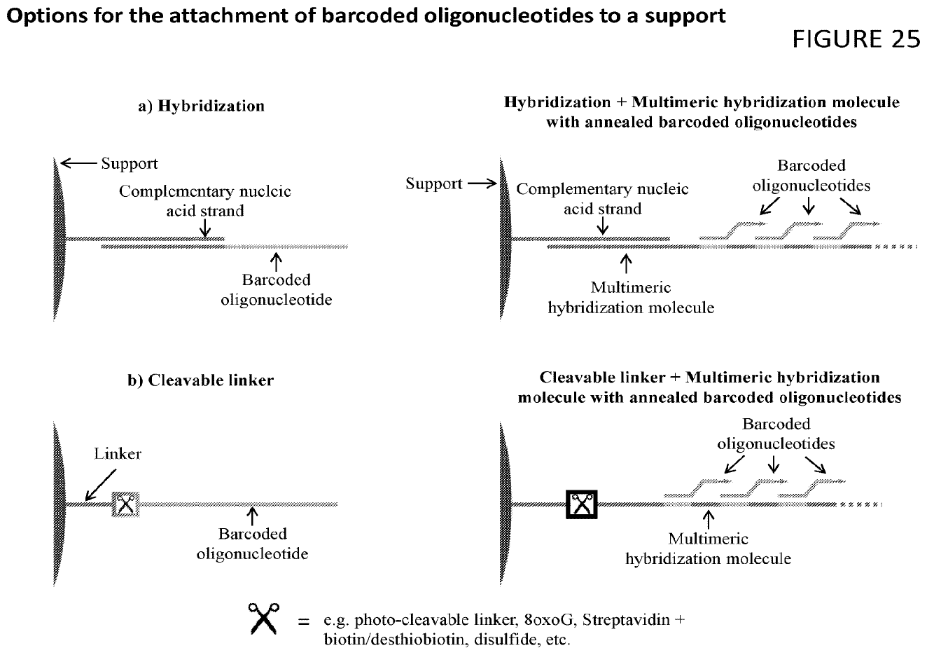

FIG. 25 illustrates options for the attachment of barcoded oligonucleotides to a support (e.g. a bead). Barcoded oligonucleotides may be linked to a support in various ways. For example, a barcoded oligonucleotide may be directly linked to the support through hybridization to a complementary nucleic acid strand directly attached to the support (see (a)) or though a linker (e.g. a cleavable linker) attached to the support (see (b)). Barcoded oligonucleotides may be linked indirectly to the support through a multimeric hybridization molecule (e.g. a multimeric barcode molecule), which may hybridize to a complementary nucleic acid strand that is directly attached to the support (see (c)) or be directly linked by a linker (e.g. a cleavable linker) that is attached to the support (see (d)).

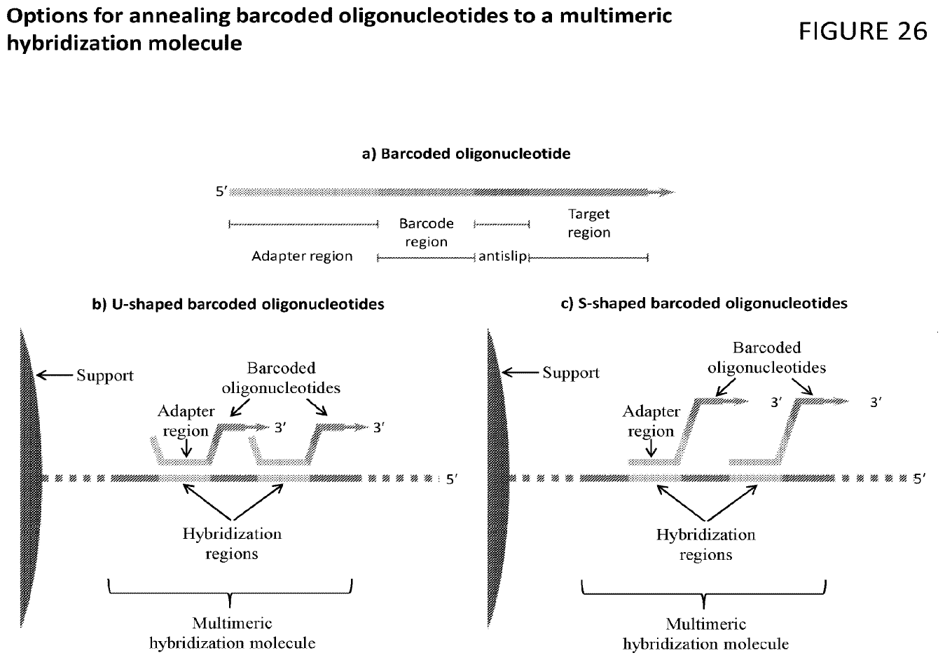

FIG. 26 illustrates options for annealing barcoded oligonucleotides to a multimeric hybridization molecule. The designs of the multimeric hybridization molecule (e.g. the multimeric barcode molecule) and the barcoded oligonucleotides are interdependent. FIG. 26(a) shows the overall structure of of a barcoded oligonucleotide with its sections (adapter region, barcode region, optional antislip region, and target region). FIGS. 26(b) and (c) who the hybridization of barcoded oligonucleotides (via their adapter regions) to the hybridization regions of multimeric hybridization molecules. The adapter region (i.e. annealing region) of a barcoded oligonucleotide can be longer than the complementary sequence of the multimeric hybridization molecule and this means that, for example, it is possible to envisage two conformations, a “U-shaped” (FIG. 26(b)) and an “S-shaped” (FIG. 26(c)). These two conformations (and all the versions in-between) are sterically different and provide advantages for different applications of the reagents.

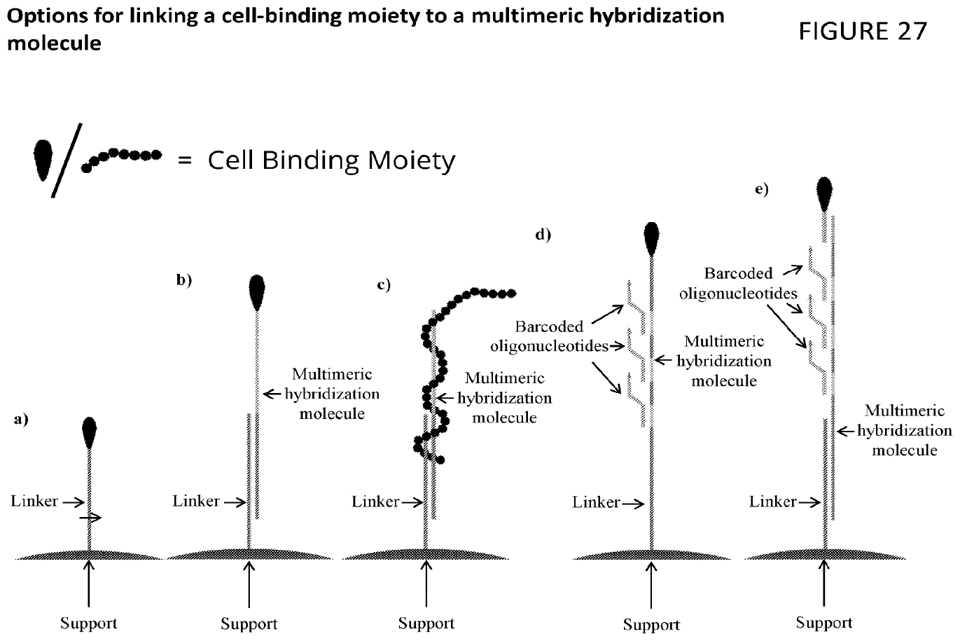

FIG. 27 illustrates options for linking a cell-binding moiety to a barcoded oligonucleotide or a multimeric hybridization molecule. The cell-binding moiety may be directly attached to the support via a linker (e.g. an oligonucleotide) (see FIG. 27(a)). The cell-binding moiety may be attached to a multimeric hybridization molecule (e.g. a multimeric barcode molecule) which is then attached to the support (see FIG. 27(d)). The cell-binding moiety may be indirectly attached to the support, for example: the cell-binding moiety may be attached to a multimeric hybridization molecule (e.g. a multimeric barcode molecule) that is annealed to an oligonucleotide attached to the support (see FIGS. 27(b) and (e)); or the cell-binding moiety may be linked to a multimeric hybridization molecule (e.g. a multimeric barcode molecule) via electrostatic interactions (see FIG. 27(c)).

### 25. EXAMPLES

### Materials and Methods

### Method 1—Synthesis of a Library of Nucleic Acid Barcode Molecules

### Synthesis of Double-Stranded Sub-Barcode Molecule Library

In a PCR tube, 10 microliters of 10 micromolar BC_MX3 (an equimolar mixture of all sequences in SEQ ID NO: 18 to 269) were added to 10 microliters of 10 micromolar BC_ADD_TP1 (SEQ ID NO: 1), plus 10 microliters of 10× CutSmart Buffer (New England Biolabs) plus 1.0 microliter of 10 millimolar deoxynucleotide triphosphate nucleotide mix (Invitrogen) plus 68 microliters H₂O, to final volume of 99 microliters. The PCR tube was placed on a thermal cycler and incubated at 75° C. for 5 minutes, then slowly annealed to 4° C., then held 4′° C., then placed on ice. 1.0 microliter of Klenow polymerase fragment (New England Biolabs; at 5 U/uL) was added to the solution and mixed. The PCR tube was again placed on a thermal cycler and incubated at 25° C. for 15 minutes, then held at 4° C. The solution was then purified with a purification column (Nucleotide Removal Kit; Qiagen), eluted in 50 microliters H₂O, and quantitated spectrophotometrically.

### Synthesis of Double-Stranded Downstream Adapter Molecule

In a PCR tube, 0.5 microliters of 100 micromolar BC_ANC_TP1 (SEQ ID NO: 2) were added to 0.5 microliters of 100 micromolar BC_ANC_BT1 (SEQ ID NO: 3), plus 20 microliters of 10× CutSmart Buffer (New England Biolabs) plus 178 microliters H₂O, to final volume of 200 microliters. The PCR tube was placed on a thermal cycler and incubated at 95° C. for 5 minutes, then slowly annealed to 4° C., then held 4° C., then placed on ice, then stored at −20° C.

### Ligation of Double-Stranded Sub-Barcode Molecule Library to Double-Stranded Downstream Adapter Molecule

In a 1.5 milliliter Eppendorf tube, 1.0 microliter of Double-Stranded Downstream Adapter Molecule solution was added to 2.5 microliters of Double-Stranded Sub-Barcode Molecule Library, plus 2.0 microliters of 10×T4 DNA Ligase buffer, and 13.5 microliters H₂O to final volume of 19 microliters. 1.0 microliter of T4 DNA Ligase (New England Biolabs; high concentration) was added to the solution and mixed. The tube was incubated at room temperature for 60 minutes, then purified with 1.8× volume (34 microliters) Ampure XP Beads (Agencourt; as per manufacturer's instructions), and eluted in 40 microliters H₂O.

### PCR Amplification of Ligated Library

In a PCR tube, 2.0 microliters of Ligated Library were added to 2.0 microliters of 50 micromolar BC_FWD_PR1 (SEQ ID NO: 4), plus 2.0 microliters of 50 micromolar BC_REV_PR1 (SEQ ID NO: 5), plus 10 microliters of 10× Taq PCR Buffer (Qiagen) plus 2.0 microliter of 10 millimolar deoxynucleotide triphosphate nucleotide mix (Invitrogen) plus 81.5 microliters H₂O, plus 0.5 microliters Qiagen Taq Polymerase (at 5 U/uL) to final volume of 100 microliters. The PCR tube was placed on a thermal cycler and amplified for 15 cycles of: 95° C. for 30 seconds, then 59° C. for 30 seconds, then 72° C. for 30 seconds; then held at 4° C. The solution was then purified with 1.8× volume (180 microliters) Ampure XP Beads (Agencourt; as per manufacturer's instructions), and eluted in 50 microliters H₂O.

### Uracil Glycosylase Enzyme Digestion

To an eppendorf tube 15 microliters of the eluted PCR amplification, 1.0 microliters H₂O, plus 2.0 microliters 10× CutSmart Buffer (New England Biolabs), plus 2.0 microliter of USER enzyme solution (New England Biolabs) was added and mixed. The tube was incubated at 37° C. for 60 minutes, then the solution was purified with 1.8× volume (34 microliters) Ampure XP Beads (Agencourt; as per manufacturer's instructions), and eluted in 34 microliters H₂O.

### Mlyl Restriction Enzyme Cleavage

To the eluate from the previous (glycosylase digestion) step, 4.0 microliters 10× CutSmart Buffer (New England Biolabs), plus 2.0 microliter of Mlyl enzyme (New England Biolabs, at 5 U/uL) was added and mixed. The tube was incubated at 37° C. for 60 minutes, then the solution was purified with 1.8× volume (72 microliters) Ampure XP Beads (Agencourt; as per manufacturer's instructions), and eluted in 40 microliters H₂O.

### Ligation of Sub-Barcode Library to Mlyl-Cleaved Solution

In a 1.5 milliliter Eppendorf tube, 10 microliter of Mlyl-Cleaved Solution solution was added to 2.5 microliters of Double-Stranded Sub-Barcode Molecule Library, plus 2.0 microliters of 10× T4 DNA Ligase buffer, and 4.5 microliters H₂O to final volume of 19 microliters. 1.0 microliter of T4 DNA Ligase (New England Biolabs; high concentration) was added to the solution and mixed. The tube was incubated at room temperature for 60 minutes, then purified with 1.8× volume (34 microliters) Ampure XP Beads (Agencourt; as per manufacturer's instructions), and eluted in 40 microliters H₂O.

### Repeating Cycles of Sub-Barcode Addition

The experimental steps of: 1) Ligation of Sub-Barcode Library to Mlyl-Cleaved Solution, 2) PCR Amplification of Ligated Library, 3) Uracil Glycosylase Enzyme Digestion, and 4) Mlyl Restriction Enzyme Cleavage were repeated, in sequence, for a total of five cycles.

### Synthesis of Double-Stranded Upstream Adapter Molecule

In a PCR tube, 1.0 microliters of 100 micromolar BC_USO_TP1 (SEQ ID NO: 6) were added to 1.0 microliters of 100 micromolar BC_USO_BT1 (SEQ ID NO: 7), plus 20 microliters of 10× CutSmart Buffer (New England Biolabs) plus 178 microliters H₂O, to final volume of 200 microliters. The PCR tube was placed on a thermal cycler and incubated at 95° C. for 60 seconds, then slowly annealed to 4° C., then held 4° C., then placed on ice, then stored at −20° C.

### Ligation of Double-Stranded Upstream Adapter Molecule

In a 1.5 milliliter Eppendorf tube, 3.0 microliters of Upstream Adapter solution were added to 10.0 microliters of final (after the fifth cycle) Mlyl-Cleaved solution, plus 2.0 microliters of 10×T4 DNA Ligase buffer, and 5.0 microliters H₂O to final volume of 19 microliters. 1.0 microliter of T4 DNA Ligase (New England Biolabs; high concentration) was added to the solution and mixed. The tube was incubated at room temperature for 60 minutes, then purified with 1.8× volume (34 microliters) Ampure XP Beads (Agencourt; as per manufacturer's instructions), and eluted in 40 microliters H₂O.

### PCR Amplification of Upstream Adapter-Ligated Library

In a PCR tube, 6.0 microliters of Upstream Adapter-Ligated Library were added to 1.0 microliters of 100 micromolar BC_CS_PCR_FWD1 (SEQ ID NO: 8), plus 1.0 microliters of 100 micromolar BC_CS_PCR_REV1 (SEQ ID NO: 9), plus 10 microliters of 10× Taq PCR Buffer (Qiagen) plus 2.0 microliter of 10 millimolar deoxynucleotide triphosphate nucleotide mix (Invitrogen) plus 73.5 microliters H₂O, plus 0.5 microliters Qiagen Taq Polymerase (at 5 U/uL) to final volume of 100 microliters. The PCR tube was placed on a thermal cycler and amplified for 15 cycles of: 95° C. for 30 seconds, then 61° C. for 30 seconds, then 72° C. for 30 seconds; then held at 4° C. The solution, containing a library of amplified nucleic acid barcode molecules, was then purified with 1.8× volume (180 microliters) Ampure XP Beads (Agencourt; as per manufacturer's instructions). The library of amplified nucleic acid barcode molecules was then eluted in 40 microliters H₂O.

The library of amplified nucleic acid barcode molecules sythesised by the method described above was then used to assemble a library of multimeric barcode molecules as described below.

### Method 2—Assembly of a Library of Multimeric Barcode Molecules

A library of multimeric barcode molecules was assembled using the library of nucleic acid barcode molecules synthesised according to the methods of Method 1.

Primer-Extension with Forward Termination Primer and Forward Splinting Primer

In a PCR tube, 5.0 microliters of the library of amplified nucleic acid barcode molecules were added to 1.0 microliters of 100 micromolar CS_SPLT_FWD1 (SEQ ID NO: 10), plus 1.0 microliters of 5 micromolar CS_TERM_FWD1 (SEQ ID NO: 11), plus 10 microliters of 10× Thermopol Buffer (NEB) plus 2.0 microliter of 10 millimolar deoxynucleotide triphosphate nucleotide mix (Invitrogen) plus 80.0 microliters H₂O, plus 1.0 microliters Vent Exo-Minus Polymerase (New England Biolabs, at 2 U/uL) to final volume of 100 microliters. The PCR tube was placed on a thermal cycler and amplified for 1 cycle of: 95° C. for 30 seconds, then 53° C. for 30 seconds, then 72° C. for 60 seconds, then 1 cycle of: 95° C. for 30 seconds, then 50° C. for 30 seconds, then 72° C. for 60 seconds, then held at 4° C. The solution was then purified a PCR purification column (Qiagen), and eluted in 85.0 microliters H₂O.

Primer-Extension with Reverse Termination Primer and Reverse Splinting Primer

In a PCR tube, the 85.0 microliters of forward-extension primer-extension products were added to 1.0 microliters of 100 micromolar CS_SPLT_REV1 (SEQ ID NO: 12), plus 1.0 microliters of 5 micromolar CS_TERM_REV1 (SEQ ID NO: 13), plus 10 microliters of 10× Thermopol Buffer (NEB) plus 2.0 microliter of 10 millimolar deoxynucleotide triphosphate nucleotide mix (Invitrogen), plus 1.0 microliters Vent Exo-Minus Polymerase (New England Biolabs, at 2 U/uL) to final volume of 100 microliters. The PCR tube was placed on a thermal cycler and amplified for 1 cycle of: 95° C. for 30 seconds, then 53° C. for 30 seconds, then 72° C. for 60 seconds, then 1 cycle of: 95° C. for 30 seconds, then 50° C. for 30 seconds, then 72° C. for 60 seconds, then held at 4° C. The solution was then purified a PCR purification column (Qiagen), and eluted in 43.0 microliters H₂O.

Linking Primer-Extension Products with Overlap-Extension PCR

In a PCR tube were added the 43.0 microliters of reverse-extension primer-extension products, plus 5.0 microliters of 10× Thermopol Buffer (NEB) plus 1.0 microliter of 10 millimolar deoxynucleotide triphosphate nucleotide mix (Invitrogen), plus 1.0 microliters Vent Exo-Minus Polymerase (New England Biolabs, at 2 U/uL) to final volume of 50 microliters. The PCR tube was placed on a thermal cycler and amplified for 5 cycles of: 95° C. for 30 seconds, then 60° C. for 60 seconds, then 72° C. for 2 minutes; then 5 cycles of: 95° C. for 30 seconds, then 60° C. for 60 seconds, then 72° C. for 5 minutes; then 5 cycles of: 95° C. for 30 seconds, then 60° C. for 60 seconds, then 72° C. for 10 minutes; then held at 4° C. The solution was then purified with 0.8× volume (80 microliters) Ampure XP Beads (Agencourt; as per manufacturer's instructions), and eluted in 40 microliters H₂O.

### Amplification of Overlap-Extension Products

In a PCR tube were added 2.0 microliters of Overlap-Extension PCR solution, plus 1.0 microliters of 100 micromolar CS_PCR_FWD1 (SEQ ID NO: 14), plus 1.0 microliters of 100 micromolar CS_PCR_REV1 (SEQ ID NO: 15), plus 10 microliters of 10× Thermopol Buffer (NEB) plus 2.0 microliter of 10 millimolar deoxynucleotide triphosphate nucleotide mix (Invitrogen), plus 1.0 microliters Vent Exo-Minus Polymerase (New England Biolabs, at 2 U/uL), plus 83.0 microliters H₂O to final volume of 100 microliters. The PCR tube was placed on a thermal cycler and amplified for 15 cycles of: 95° C. for 30 seconds, then 58′C for 30 seconds, then 72° C. for 10 minutes; then held at 4° C. The solution was then purified with 0.8× volume (80 microliters) Ampure XP Beads (Agencourt; as per manufacturer's instructions), and eluted in 50 microliters H₂O, and quantitated spectrophotometrically.

### Gel-Based Size Selection of Amplified Overlap-Extension Products

Approximately 250 nanograms of Amplified Overlap-Extension Products were loaded and run on a 0.9% agarose gel, and then stained and visualised with ethidium bromide. A band corresponding to 1000 nucleotide size (plus and minus 100 nucleotides) was excised and purified with a gel extraction column (Gel Extraction Kit, Qiagen) and eluted in 50 microliters H₂O.

### Amplification of Overlap-Extension Products

In a PCR tube were added 10.0 microliters of Gel-Size-Selected solution, plus 1.0 microliters of 100 micromolar CS_PCR_FWD1 (SEQ ID NO: 14), plus 1.0 microliters of 100 micromolar CS_PCR_REV1 (SEQ ID NO: 15), plus 10 microliters of 10× Thermopol Buffer (NEB) plus 2.0 microliter of 10 millimolar deoxynucleotide triphosphate nucleotide mix (Invitrogen), plus 1.0 microliters Vent Exo-Minus Polymerase (New England Biolabs, at 2 U/uL) plus 75.0 microliters H₂O to final volume of 100 microliters. The PCR tube was placed on a thermal cycler and amplified for 15 cycles of: 95° C. for 30 seconds, then 58° C. for 30 seconds, then 72° C. for 4 minutes; then held at 4° C. The solution was then purified with 0.8× volume (80 microliters) Ampure XP Beads (Agencourt; as per manufacturer's instructions), and eluted in 50 microliters H₂O, and quantitated spectrophotometrically.

Selection and Amplification of Quantitatively Known Number of Multimeric Barcode Molecules Amplified gel-extracted solution was diluted to a concentration of 1 picogram per microliter, and then to a PCR tube was added 2.0 microliters of this diluted solution (approximately 2 million individual molecules), plus 0.1 microliters of 100 micromolar CS_PCR_FWD1 (SEQ ID NO: 14), plus 0.1 microliters of 100 micromolar CS_PCR_REV1 (SEQ ID NO: 15), plus 1.0 microliter 10× Thermopol Buffer (NEB) plus 0.2 microliter of 10 millimolar deoxynucleotide triphosphate nucleotide mix (Invitrogen), plus 0.1 microliters Vent Exo-Minus Polymerase (New England Biolabs, at 2 U/uL) plus 6.5 microliters H₂O to final volume of 10 microliters. The PCR tube was placed on a thermal cycler and amplified for 11 cycles of: 95° C. for 30 seconds, then 57° C. for 30 seconds, then 72° C. for 4 minutes; then held at 4° C.

To the PCR tube was added 1.0 microliters of 100 micromolar CS_PCR_FWD1 (SEQ ID NO: 14), plus 1.0 microliters of 100 micromolar CS_PCR_REV1 (SEQ ID NO: 15), plus 9.0 microliters of 10× Thermopol Buffer (NEB) plus 2.0 microliter of 10 millimolar deoxynucleotide triphosphate nucleotide mix (Invitrogen), plus 1.0 microliters Vent Exo-Minus Polymerase (New England Biolabs, at 2 U/uL) plus 76.0 microliters H₂O to final volume of 100 microliters. The PCR tube was placed on a thermal cycler and amplified for 10 cycles of: 95° C. for 30 seconds, then 57° C. for 30 seconds, then 72° C. for 4 minutes; then held at 4° C. The solution was then purified with 0.8× volume (80 microliters) Ampure XP Beads (Agencourt; as per manufacturer's instructions), and eluted in 50 microliters H₂O, and quantitated spectrophotometrically.

Method 3: Production of Single-Stranded Multimeric Barcode Molecules by In Vitro Transcription and cDNA Synthesis

This method describes a series of steps to produce single-stranded DNA strands, to which oligonucleotides may be annealed and then barcoded along. This method begins with four identical reactions performed in parallel, in which a promoter site for the T7 RNA Polymerase is appended to the 5′ end of a library of multimeric barcode molecules using an overlap-extension PCR amplification reaction. Four identical reactions are performed in parallel and then merged to increase the quantitative amount and concentration of this product available. In each of four identical PCR tubes, approximately 500 picograms of size-selected and PCR-amplified multimeric barcode molecules (as produced in the Selection and Amplification of Quantitatively Known Number of Multimeric Barcode Molecules' step of Method 2) were mixed with 2.0 microliters of 100 micromolar CS_PCR_FWD1_T7 (SEQ ID NO. 270) and 2.0 microliters of 100 micromolar CS_PCR_REV4 (SEQ ID NO. 271), plus 20.0 microliters of 10× Thermopol PCR buffer, plus 4.0 microliters of 10 millimolar deoxynucleotide triphosphate nucleotide mix, and 2.0 microliters Vent Exo Minus polymerse (at 5 units per microliter) plus water to a total volume of 200 microliters. The PCR tube was placed on a thermal cycler and amplified for 22 cycles of: 95° C. for 60 seconds, then 60° C. for 30 seconds, then 72° C. for 3 minutes; then held at 4° C. The solution from all four reactions was then purified with a gel extraction column (Gel Extraction Kit, Qiagen) and eluted in 52 microliters H₂O.

Fifty (50) microliters of the eluate was mixed with 10 microliters 10×NEBuffer 2 (NEB), plus 0.5 microliters of 10 millimolar deoxynucleotide triphosphate nucleotide mix, and 1.0 microliters Vent Exo Minus polymerse (at 5 units per microliter) plus water to a total volume of 100 microliters. The reaction was incubated for 15 minutes at room temperature, then purified with 0.8× volume (80 microliters) Ampure XP Beads (Agencourt; as per manufacturer's instructions), and eluted in 40 microliters H₂O, and quantitated spectrophotometrically.

A transcription step is then performed, in which the library of PCR-amplified templates containing T7 RNA Polymerase promoter site (as produced in the preceding step) is used as a template for T7 RNA polymerase. This comprises an amplification step to produce a large amount of RNA-based nucleic acid corresponding to the library of multimeric barcode molecules (since each input PCR molecule can serve as a template to produce a large number of cognate RNA molecules). In the subsequent step, these RNA molecules are then reverse transcribed to create the desired, single-stranded multimeric barcode molecules. Ten (10) microliters of the eluate was mixed with 20 microliters 5× Transcription Buffer (Promega), plus 2.0 microliters of 10 millimolar deoxynucleotide triphosphate nucleotide mix, plus 10 microliters of 0.1 milimolar DTT, plus 4.0 microliters SuperAseln (Ambion), and 4.0 microliters Promega T7 RNA Polymerase (at 20 units per microliter) plus water to a total volume of 100 microliters. The reaction was incubated 4 hours at 37° C., then purified with an RNEasy Mini Kit (Qiagen), and eluted in 50 micoliters H₂O, and added to 6.0 microliters SuperAseln (Ambion).

The RNA solution produced in the preceding in vitro transcription step is then reverse transcribed (using a primer specific to the 3′ ends of the RNA molecules) and then digested with RNAse H to create single-stranded DNA molecules corresponding to multimeric barcode molecules, to which oligonucleotides maybe be annealed and then barcoded along. In two identical replicate tubes, 23.5 microliters of the eluate was mixed with 5.0 microliters of 10 millimolar deoxynucleotide triphosphate nucleotide mix, plus 3.0 microliters SuperAseln (Ambion), and 10.0 microliters of 2.0 micromolar CS_PCR_REV1 (SEQ ID NO. 272) plus water to final volume of 73.5 microliters. The reaction was incubated on a thermal cycler at 65° C. for 5 minutes, then 50° C. for 60 seconds; then held at 4° C. To the tube was added 20 microliters 5× Reverse Transcription buffer (Invitrogen), plus 5.0 microliters of 0.1 milimolar DTT, and 1.75 microliters Superscript Ill Reverse Transcriptase (Invitrogen). The reaction was incubated at 55° C. for 45 minutes, then 60° C. for 5 minutes; then 70° C. for 15 minutes, then held at 4° C., then purified with a PCR Cleanup column (Qiagen) and eluted in 40 microliters H₂O.

Sixty (60) microliters of the eluate was mixed with 7.0 microliters 10×RNAse H Buffer (Promega), plus 4.0 microliters RNAse H (Promega. The reaction was incubated 12 hours at 37° C., then 95° C. for 10 minutes, then held at 4° C., then purified with 0.7× volume (49 microliters) Ampure XP Beads (Agencourt; as per manufacturer's instructions), and eluted in 30 microliters H₂O, and quantitated spectrophotometrically.

### Method 4: Production of Multimeric Barcoding Reagents Containing Barcoded Oligonucleotides

This method describes steps to produce multimeric barcoding reagents from single-stranded multimeric barcode molecules (as produced in Method 3) and appropriate extension primers and adapter oligonucleotides.

In a PCR tube, approximately 45 nanograms of single-stranded RNAse H-digested multimeric barcode molecules (as produced in the last step of Method 3) were mixed with 0.25 microliters of 10 micromolar DS_ST_05 (SEQ ID NO. 273, an adapter oligonucleotide) and 0.25 microliters of 10 micromolar US_PCR_Prm_Only_03 (SEQ ID NO. 274, an extension primer), plus 5.0 microliters of 5× Isothermal extension/ligation buffer, plus water to final volume of 19.7 microliters. In order to anneal the adapter oligonucleotides and extension primers to the multimeric barcode molecules, in a thermal cycler, the tube was incubated at 98° C. for 60 seconds, then slowly annealed to 55° C., then held at 55° C. for 60 seconds, then slowly annealed to 50° C. then held at 50° C. for 60 seconds, then slowly annealed to 20° C. at 0.1° C./sec, then held at 4° C. To the tube was added 0.3 microliters (0.625 U) Phusion Polymerase (NEB; 2 U/uL) 2.5 microliters (100 U) Taq DNA Ligase (NEB; 40 U/uL); and 2.5 microliters 100 milimolar DTT. In order to extend the extension primer(s) across the adjacent barcode region(s) of each multimeric barcode molecule, and then to ligate this extension product to the phosphorylated 5′ end of the adapter oligonucleotide annealed to the downstream thereof, the tube was then incubated at 50° C. for 3 minutes, then held at 4° C. The reaction was then purified with a PCR Cleanup column (Qiagen) and eluted in 30 microliters H₂O, and quantitated spectrophotometrically.

### Method 5: Production of Synthetic DNA Templates of Known Sequence

This method describes a technique to produce synthetic DNA templates with a large number of tandemly-repeated, co-linear molecular sequence identifiers, by circularizing and then tandemly amplifying (with a processive, strand-displacing polymerase) oligonucleotides containing said molecular sequence identifiers. This reagent may then be used to evaluate and measure the multimeric barcoding reagents described herein.

In a PCR was added 0.4 microliters of 1.0 micromolar Syn_Temp_01 (SEQ ID NO. 275) and 0.4 microliters of 1.0 micromolar ST_Splint_02 (SEQ ID NO. 276) and 10.0 microliters of 10×NEB CutSmart buffer. On a thermal cycler, the tube was incubated at 95° C. for 60 seconds, then held at 75° C. for 5 minutes, then slowly annealed to 20° C. then held at 20° C. for 60 seconds, then held at 4° C. To circularize the molecules through an intramolecular ligation reaction, the tube was then added 10.0 microliters ribo-ATP and 5.0 microliters T4 DNA Ligase (NEB; High Concentration). The tube was then incubated at room temperature for 30 minutes, then at 65° C. for 10 minutes, then slowly annealed to 20° C. then held at 20° C. for 60 seconds, then held at 4° C. To each tube was then added 10×NEB CutSmart buffer, 4.0 microliters of 10 millimolar deoxynucleotide triphosphate nucleotide mix, and 1.5 microliters of diluted phi29 DNA Polymerase (NEB; Diluted 1:20 in 1× CutSmart buffer) plus water to a total volume of 200 microliters. The reaction was incubated at 30° C. for 5 minutes, then held at 4° C., then purified with 0.7× volume (140 microliters) Ampure XP Beads (Agencourt; as per manufacturer's instructions), and eluted in 30 microliters H₂O, and quantitated spectrophotometrically.

Method 6: Barcoding Synthetic DNA Templates of Known Sequence with Multimeric Barcoding Reagents Containing Barcoded Oligonucleotides

In a PCR tube were added 10.0 microliters 5× Phusion HF buffer (NEB), plus 1.0 microliters 10 millimolar deoxynucleotide triphosphate nucleotide mix, plus 2.0 microliters (10 nanograms) 5.0 nanogram/microliters Synthetic DNA Templates of Known Sequence (as produced by Method 5), plus water to final volume of 42.5 microliters. The tube was then incubated at 98° C. for 60 seconds, then held at 20° C. To the tube was added 5.0 microliters of 5.0 picogram/microliter Multimeric Barcoding Reagents Containing Barcoded Oligonucleotides (as produced by Method 4). The reaction was then incubated at 70° C. for 60 seconds, then slowly annealed to 60° C., then 60° C. for five minutes, then slowly annealed to 55° C., then 55° C. for five minutes, then slowly annealed to 50° C., then 50° C. for five minutes, then held at 4° C. To the reaction was added 0.5 microliters of Phusion Polymerase (NEB), plus 2.0 microliters 10 uM SynTemp_PE2_B1_Short1 (SEQ ID NO. 277, a primer that is complementary to part of the extension products produced by annealing and extending the multimeric barcoding reagents created by Method 4 along the synthetic DNA templates created by Method 5, serves as a primer for the primer-extension and then PCR reactions described in this method). Of this reaction, a volume of 5.0 microliters was added to a new PCR tube, which was then incubated for 30 seconds at 55° C., 30 seconds 60° C., and 30 seconds 72′° C., then followed by 10 cycles of: 98° C. then 65° C. then 72° C. for 30 seconds each, then held at 4° C. To each tube was then added 9.0 microliters 5× Phusion buffer, plus 1.0 microliters 10 millimolar deoxynucleotide triphosphate nucleotide mix, plus 1.75 microliters 10 uM SynTemp_PE2_B1_Short1 (SEQ ID NO. 277), plus 1.75 microliters 10 UM US_PCR_Prm_Only_02 (SEQ ID NO. 278, a primer partially complementary to the extension primer employed to generate the multimeric barcoding reagents as per Method 4, and serving as the ‘forward’ primer in this PCR amplification reaction), plus 0.5 microliters Phusion Polymerase (NEB), plus water to final volume of 50 microliters. The PCR tube was placed on a thermal cycler and amplified for 24 cycles of: 98° C. for 30 seconds, then 72° C. for 30 seconds; then held at 4° C., then purified with 1.2× volume (60 microliters) Ampure XP Beads (Agencourt; as per manufacturer's instructions), and eluted in 30 microliters H₂O, and quantitated spectrophotometrically.

The resulting library was then barcoded for sample identification by a PCR-based method, amplified, and sequenced by standard methods using a 150-cycle, mid-output NextSeq flowcell (Illumina), and demultiplexed informatically for further analysis.

Method 7: Barcoding Synthetic DNA Templates of Known Sequence with Multimeric Barcoding Reagents and Separate Adapter Oligonucleotides

To anneal and extend adapter oligonucleotides along the synthetic DNA templates, in a PCR tube were added 10.0 microliters 5× Phusion HF buffer (NEB), plus 1.0 microliters 10 millimolar deoxynucleotide triphosphate nucleotide mix, plus 5.0 microliters (25 nanograms) 5.0 nanogram/microliters Synthetic DNA Templates of Known Sequence (as produced by Method 5), plus 0.25 microliters of 10 micromolar DS_ST_05 (SEQ ID NO. 273, an adapter oligonucleotide), plus water to final volume of 49.7 microliters. On a thermal cycler, the tube was incubated at 98° C. for 2 minutes, then 63° C. for 1 minute, then slowly annealed to 60° C. then held at 60° C. for 1 minute, then slowly annealed to 57° C. then held at 57° C. for 1 minute, then slowly annealed to 54° C. then held at 54° C. for 1 minute, then slowly annealed to 50° C. then held at 50° C. for 1 minute, then slowly annealed to 45° C. then held at 45° C. for 1 minute, then slowly annealed to 40° C. then held at 40° C. for 1 minute, then held at 4′C. To the tube was added 0.3 microliters Phusion Polymerase (NEB), and the reaction was incubated at 45° C. for 20 seconds, then 50° C. for 20 seconds, then 55° C. for 20 seconds, 60° C. for 20 seconds, then 72° C. for 20 seconds, then held at 4° C.; the reaction was then purified with 0.8× volume (40 microliters) Ampure XP Beads (Agencourt; as per manufacturer's instructions), and eluted in 30 microliters H₂O, and quantitated spectrophotometrically.

In order to anneal adapter oligonucleotides (annealed and extended along the synthetic DNA templates as in the previous step) to multimeric barcode molecules, and then to anneal and then extend extension primer(s) across the adjacent barcode region(s) of each multimeric barcode molecule, and then to ligate this extension product to the phosphorylated 5′ end of the adapter oligonucleotide annealed to the downstream thereof, to a PCR tube was added 10 microliters of the eluate from the previous step (containing the synthetic DNA templates along which the adapter oligonucleotides have been annealed and extended), plus 3.0 microliters of a 50.0 nanomolar solution of RNAse H-digested multimeric barcode molecules (as produced in the last step of Method 3), plus 6.0 microliters of 5× Isothermal extension/ligation buffer, plus water to final volume of 26.6 microliters. On a thermal cycler, the tube was incubated at 70° C. for 60 seconds, then slowly annealed to 60° C., then held at 60° C. for 5 minutes, then slowly annealed to 55° C. then held at 55° C. for 5 minutes, then slowly annealed to 50° C. at 0.1° C./sec then held at 50° C. for 30 minutes, then held at 4° C. To the tube was added 0.6 microliters 10 UM US_PCR_Prm_Only_02 (SEQ ID NO: 278, an extension primer), and the reaction was incubated at 50° C. for 10 minutes, then held at 4° C. To the tube was added 0.3 microliters (0.625 U) Phusion Polymerase (NEB: 2 U/uL) 2.5 microliters (100 U) Taq DNA Ligase (NEB; 40 U/uL); and 2.5 microliters 100 milimolar DTT. The tube was then incubated at 50° C. for 5 minutes, then held at 4° C. The reaction was then purified with 0.7× volume (21 microliters) Ampure XP Beads (Agencourt; as per manufacturer's instructions), and eluted in 30 microliters H₂O, and quantitated spectrophotometrically.

To a new PCR tube was add 25.0 microliters of the eluate, plus 10.0 microliters 5× Phusion HF buffer (NEB), plus 1.0 microliters 10 millimolar deoxynucleotide triphosphate nucleotide mix, plus 2.0 microliters 10 uM SynTemp_PE2_B1_Short1 (SEQ ID NO: 277; a primer that is complementary to part of the extension products produced by the above steps; serves as a primer for the primer-extension and then PCR reactions described here), plus 0.5 uL Phusion Polymerase (NEB), plus water to final volume of 49.7 microliters. Of this reaction, a volume of 5.0 microliters was added to a new PCR tube, which was then incubated for 30 seconds at 55° C., 30 seconds 60° C., and 30 seconds 72° C., then followed by 10 cycles of: 98° C. then 65° C. then 72° C. for 30 seconds each, then held at 4° C. To each tube was then added 9.0 microliters 5× Phusion buffer, plus 1.0 microliters 10 millimolar deoxynucleotide triphosphate nucleotide mix, plus 1.75 microliters 10 uM SynTemp_PE2_B1_Short1 (SEQ ID NO: 277), plus 1.75 microliters 10 UM US_PCR_Prm_Only_02 (SEQ ID NO: 278), plus 0.5 microliters Phusion Polymerase (NEB), plus water to final volume of 50 microliters. The PCR tube was placed on a thermal cycler and amplified for 24 cycles of: 98° C. for 30 seconds, then 72° C. for 30 seconds; then held at 4° C., then purified with 1.2× volume (60 microliters) Ampure XP Beads (Agencourt; as per manufacturer's instructions), and eluted in 30 microliters H₂O, and quantitated spectrophotometrically.

The resulting library was then barcoded for sample identification by a PCR-based method, amplified, and sequenced by standard methods using a 150-cycle, mid-output NextSeq flowcell (Illumina), and demultiplexed informatically for further analysis.

Method 9: Barcoding Genomic DNA Loci with Multimeric Barcoding Reagents Containing Barcoded Oligonucleotides

This method describes a framework for barcoding targets within specific genomic loci (e.g. barcoding a number of exons within a specific gene) using multimeric barcoding reagents that contain barcoded oligonucleotides. First, a solution of Multimeric Barcode Molecules was produced by In Vitro Transcription and cDNA Synthesis (as described in Method 3). Then, solutions of multimeric barcoding reagents containing barcoded oligonucleotides was produced as described in Method 4, with a modification made such that instead of using an adapter oligonucleotide targeting a synthetic DNA template (i.e. DS_ST_05, SEQ ID NO: 273, as used in Method 4), adapter oligonucleotides targeting the specific genomic loci were included at that step. Specifically, a solution of multimeric barcoding reagents containing appropriate barcoded oligonucleotides was produced individually for each of three different human genes: BRCA1 (containing 7 adapter oligonucleotides, SEQ ID NOs 279-285), HLA-A (containing 3 adapter oligonucleotides, SEQ ID NOs 286-288), and DQB1 (containing 2 adapter oligonucleotides, SEQ ID NOs 289-290). The process of Method 4 was conducted for each of these three solutions as described above. These three solutions were then merged together, in equal volume, and diluted to a final, total concentration all barcoded oligonucleotides of approximately 50 nanomolar.

In a PCR tube were plus 2.0 microliters 5× Phusion HF buffer (NEB), plus 1.0 microliter of 100 nanogram/microliter human genomic DNA (NA12878 from Coriell Institute) to final volume of 9.0 microliters. In certain variant versions of this protocol, the multimeric barcoding reagents (containing barcoded oligonucleotides) were also added at this step, prior to the high-temperature 98° C. incubation. The reaction was incubated at 98° C. for 120 seconds, then held at 4° C. To the tube was added 1.0 microliters of the above 50 nanomolar solution of multimeric barcode reagents, and then the reaction was incubated for 1 hour at 55° C., then 1 hour at 50° C., then 1 hour at 45° C., then held at 4° C. (Note that for certain samples, this last annealing process was extended to occur overnight, for a total of approximately 4 hours per temperature step).

In order to add a reverse universal priming sequence to each amplicon sequence (and thus to enable subsequent amplification of the entire library at once, using just one forward and one reverse amplification primer), the reaction was diluted 1:100, and 1.0 microliter of the resulting solution was added in a new PCR tube to 20.0 microliters 5× Phusion HF buffer (NEB), plus 2.0 microliters 10 millimolar deoxynucleotide triphosphate nucleotide mix, plus 1.0 microliters a reverse-primer mixture (equimolar concentration of SEQ ID Nos 291-303, each primer at 5 micromolar concentration), plus 1.0 uL Phusion Polymerase (NEB), plus water to final volume of 100 microliters. The reaction was incubated at 53° C. for 30 seconds, 72° C. for 45 seconds, 98° C. for 90 seconds, then 68° C. for 30 seconds, then 64° C. for 30 seconds, then 72° C. for 30 seconds; then held at 4° C. The reaction was then purified with 0.8× volume (80 microliters) Ampure XP Beads (Agencourt; as per manufacturer's instructions), and eluted in 30 microliters H₂O, and quantitated spectrophotometrically.

The resulting library was then barcoded for sample identification by a PCR-based method, amplified, and sequenced by standard methods using a 150-cycle, mid-output NextSeq flowcell (Illumina), and demultiplexed informatically for further analysis.

### Method 10—Sequencing the Library of Multimeric Barcode Molecules

Preparing Amplified Selected Molecules for Assessment with High-Throughput Sequencing

To a PCR tube was added 1.0 microliters of the amplified selected molecule solution, plus 1.0 microliters of 100 micromolar CS_SQ_AMP_REV1 (SEQ ID NO: 16), plus 1.0 microliters of 100 micromolar US_PCR_Prm_Only_02 (SEQ ID NO: 17), plus 10 microliters of 10× Thermopol Buffer (NEB) plus 2.0 microliter of 10 millimolar deoxynucleotide triphosphate nucleotide mix (Invitrogen), plus 1.0 microliters Vent Exo-Minus Polymerase (New England Biolabs, at 2 U/uL) plus 84.0 microliters H₂O to final volume of 100 microliters. The PCR tube was placed on a thermal cycler and amplified for 3 cycles of: 95° C. for 30 seconds, then 56° C. for 30 seconds, then 72° C. for 3 minutes; then held at 4° C. The solution was then purified with 0.8× volume (80 microliters) Ampure XP Beads (Agencourt; as per manufacturer's instructions), and eluted in 85 microliters H₂O.

This solution was then added to a new PCR tube, plus 1.0 microliters of 100 micromolar Illumina_PE1, plus 1.0 microliters of 100 micromolar Illumina_PE2, plus 10 microliters of 10× Thermopol Buffer (NEB) plus 2.0 microliter of 10 millimolar deoxynucleotide triphosphate nucleotide mix (Invitrogen), plus 1.0 microliters Vent Exo-Minus Polymerase (New England Biolabs, at 2 U/uL) to final volume of 100 microliters. The PCR tube was placed on a thermal cycler and amplified for 4 cycles of: 95° C. for 30 seconds, then 64° C. for 30 seconds, then 72° C. for 3 minutes; then 18 cycles of: 95° C. for 30 seconds, then 67° C. for 30 seconds, then 72° C. for 3 minutes; then held at 4° C. The solution was then purified with 0.8× volume (80 microliters) Ampure XP Beads (Agencourt; as per manufacturer's instructions), and eluted in 40 microliters H₂O.

High-throughput Illumina sequencing was then performed on this sample using a MiSeq sequencer with paired-end. 250-cycle V2 sequencing chemistry.

### Method 11—Assessment of Multimeric Nature of Barcodes Annealed and Extended Along Single Synthetic Template DNA Molecules

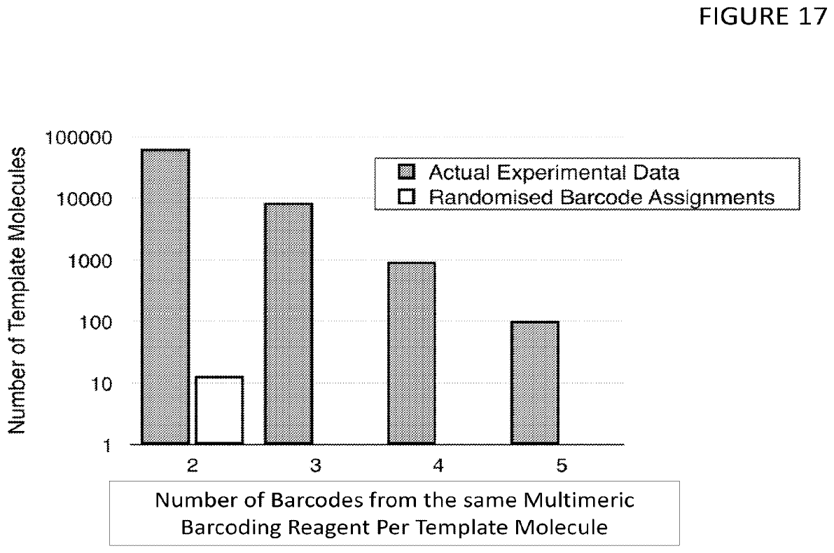

A library of barcoded synthetic DNA templates was created using a solution of multimeric barcoding reagents produced according to a protocol as described generally in Method 3 and Method 4, and using a solution of synthetic DNA templates as described in Method 5, and using a laboratory protocol as described in Method 6; the resulting library was then barcoded for sample identification by a PCR-based method, amplified, and sequenced by standard methods using a 150-cycle, mid-output NextSeq flowcell (Illumina), and demultiplexed informatically for further analysis. The DNA sequencing results from this method were then compared informatically with data produced from Method 10 to assess the degree of overlap between the multimeric barcoding of synthetic DNA templates and the arrangement of said barcodes on individual multimeric barcoding reagents (the results are shown in FIG. 17).

### Results

### Structure and Expected Sequence Content of Each Sequence Multimeric Barcoding Reagent Molecule

The library of multimeric barcode molecules synthesised as described in Methods 1 to 3 was prepared for high-throughput sequencing, wherein each molecule sequenced includes a contiguous span of a specific multimeric barcode molecule (including one or more barcode sequences, and one or more associate upstream adapter sequences and/or downstream adapter sequences), all co-linear within the sequenced molecule. This library was then sequenced with paired-end 250 nucleotide reads on a MiSeq sequencer (Illumina) as described. This yielded approximately 13.5 million total molecules sequenced from the library, sequenced once from each end, for a total of approximately 27 million sequence reads.

Each forward read is expected to start with a six nucleotide sequence, corresponding to the 3′ end of the upstream adapter: TGACCT

This forward read is followed by the first barcode sequence within the molecule (expected to be 20 nt long).

This barcode is then followed by an ‘intra-barcode sequence’ (in this case being sequenced in the “forward” direction (which is 82 nucleotides including both the downstream adapter sequence and upstream adapter sequence in series):

ATACCTGACTGCTCGTCAGTTGAGCGAATTCCGTATGGTGGTACACACC
TACACTACTCGGACGCTCTTCCGATCTTGACCT

Within the 250 nucleotide forward read, this will then be followed by a second barcode, another intra-barcode sequence, and then a third barcode, and then a fraction of another intra-barcode sequence.

Each reverse read is expected to start with a sequence corresponding to the downstream adapter sequence: GCTCAACTGACGAGCAGTCAGGTAT

This reverse read is then followed by the first barcode coming in from the opposite end of the molecule (also 20 nucleotides long, but sequenced from the opposite strand of the molecule and thus of the inverse orientation to those sequenced by the forward read)

This barcode is then followed by the ‘intra-barcode sequence’ but in the inverse orientation (as it is on the opposite strand):

AGGTCAAGATCGGAAGAGCGTCCGAGTAGTGTAGGTGTGTACCACCATA
CGGAATTCGCTCAACTGACGAGCAGTCAGGTAT

Likewise this 250 nucleotide reverse read will then be followed by a second barcode, another intra-barcode sequence, and then a third barcode, and then a fraction of another intra-barcode sequence.

### Sequence Extraction and Analysis

With scripting in Python, each associated pair of barcode and flanking upstream-adapter and downstream-adapter sequence were isolated, with each individual barcode sequence of each barcode molecule then isolated, and each barcode sequence that was sequenced within the same molecule being annotated as belonging to the same multimeric barcode molecule in the library of multimeric barcode molecules. A simple analysis script (Networkx; Python) was employed to determine overall multimeric barcode molecule barcode groups, by examining overlap of barcode-barcode pairs across different sequenced molecules. Several metrics of this data were made, including barcode length, sequence content, and the size and complexity of the multimeric barcode molecules across the library of multimeric barcode molecules.

Number of Nucleotides within Each Barcode Sequence

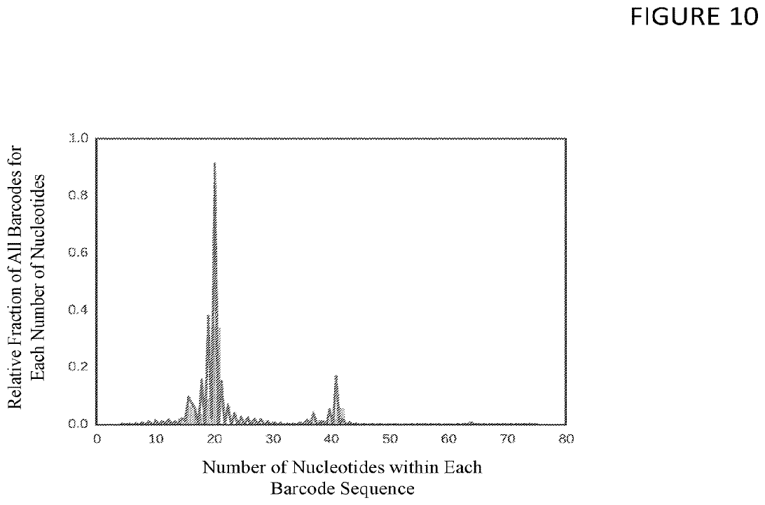

Each individual barcode sequence from each barcode molecule, contained within each Illumina-sequenced molecule was isolated, and the total length of each such barcode was determined by counting the number of nucleotides between the upstream adapter molecule sequence, and the downstream adapter molecule sequence. The results are shown in FIG. 10.

The overwhelming majority of barcodes are 20 nucleotides long, which corresponds to five additions of our four-nucleotide-long sub-barcode molecules from our double-stranded sub-barcode library. This is thus the expected and desired result, and indicates that each ‘cycle’ of: Ligation of Sub-Barcode Library to Mlyl-Cleaved Solution, PCR Amplification of the Ligated Library, Uracil Glycosylase Enzyme Digestion, and Mlyl Restriction Enzyme Cleavage, was successful and able to efficiently add new four-nucleotide sub-barcode molecules at each cycle, and then was successfully able to amplify and carry these molecules forward through the protocol for continued further processing, including through the five total cycles of sub-barcode addition, to make the final, upstream-adapter-ligated libraries.

We also used this sequence analysis method to quantitate the total number of unique barcodes in total, across all sequenced multimeric barcode molecules: this amounted to 19,953,626 total unique barcodes, which is essentially identical to the 20 million barcodes that would be expected, given that we synthesised 2 million multimeric barcode molecules, each with approximately 10 individual barcode molecules.

Together, this data and analysis thus shows that the methods of creating complex, combinatoric barcodes from sub-barcode sequences is effective and useful for the purpose of synthesising multimeric barcode molecules.

### Total Number of Unique Barcode Molecules in Each Multimeric Barcode Molecule

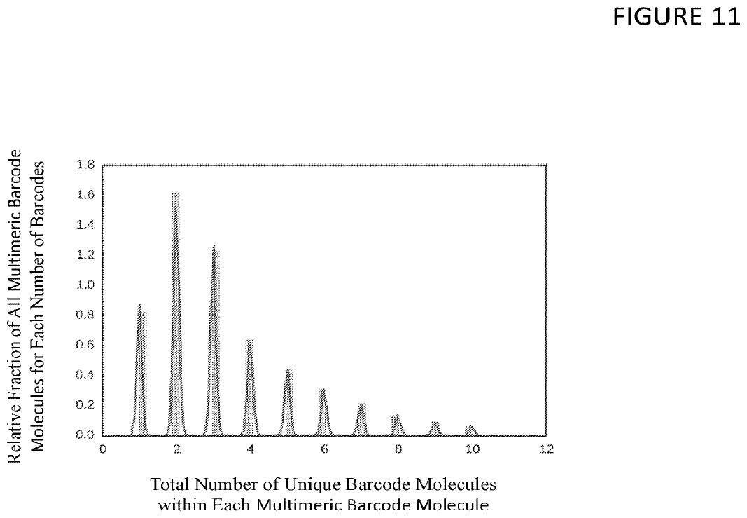

FIG. 11 shows the results of the quantification of the total number of unique barcode molecules (as determined by their respective barcode sequences) in each sequenced multimeric barcode molecule. As described above, to do this we examined, in the first case, barcode sequences which were present and detected within the same individual molecules sequenced on the sequencer. We then employed an additional step of clustering barcode sequences further, wherein we employed a simple network analysis script (Networkx) which can determine links between individual barcode sequences based both upon explicit knowledge of links (wherein the barcodes are found within the same, contiguous sequenced molecule), and can also determine ‘implicit’ links, wherein two or more barcodes, which are not sequenced within the same sequenced molecule, instead both share a direct link to a common, third barcode sequence (this shared, common link thus dictating that the two first barcode sequences are in fact located on the same multimeric barcode molecule).

This figure shows that the majority of multimeric barcode molecules sequenced within our reaction have two or more unique barcodes contained therein, thus showing that, through our Overlap-Extension PCR linking process, we are able to link together multiple barcode molecules into multimeric barcode molecules. Whilst we would expect to see more multimeric barcode molecules exhibiting closer to the expected number of barcode molecules (10), we expect that this observed effect is due to insufficiently high sequencing depth, and that with a greater number of sequenced molecules, we would be able to observe a greater fraction of the true links between individual barcode molecules. This data nonetheless suggest that the fundamental synthesis procedure we describe here is efficacious for the intended purpose.

### Representative Multimeric Barcode Molecules

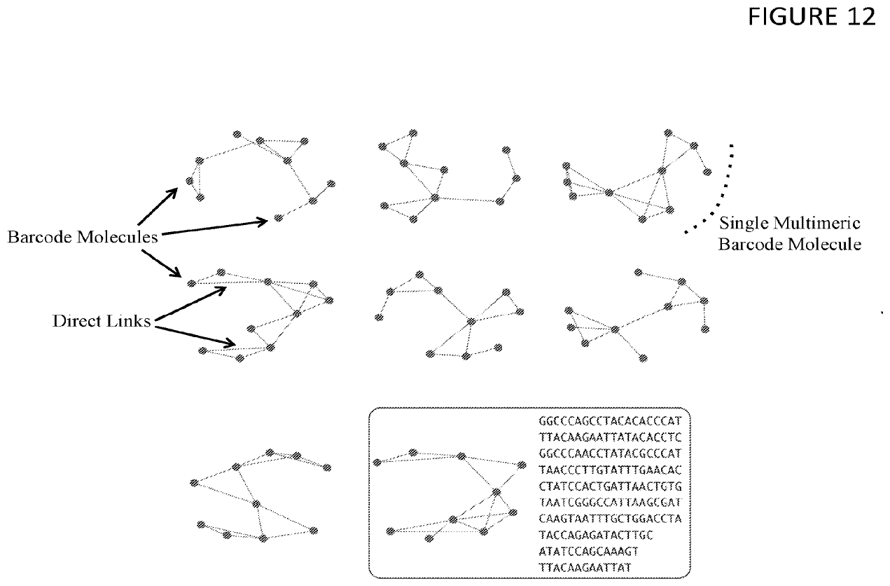

FIG. 12 shows representative multimeric barcode molecules that have been detected by our analysis script. In this figure, each ‘node’ is a single barcode molecule (from its associated barcode sequence), each line is a ‘direct link’ between two barcode molecules that have been sequenced at least once in the same sequenced molecule, and each cluster of nodes is an individual multimeric barcode molecule, containing both barcodes with direct links and those within implicit, indirect links as determined by our analysis script. The inset figure includes a single multimeric barcode molecule, and the sequences of its constituent barcode molecules contained therein.

This figure illustrates the multimeric barcode molecule synthesis procedure: that we are able to construct barcode molecules from sub-barcode molecule libraries, that we are able to link multiple barcode molecules with an overlap-extension PCR reaction, that we are able to isolate a quantitatively known number of individual multimeric barcode molecules, and that we are able to amplify these and subject them to downstream analysis and use.

Barcoding Synthetic DNA Templates of Known Sequence with (i) Multimeric Barcoding Reagents Containing Barcoded Oligonucleotides, and (ii) Multimeric Barcoding Reagents and Separate Adapter Oligonucleotides

### Sequence Extraction and Analysis

With scripting in Python and implemented in an Amazon Web Services (AWS) framework, for each sequence read following sample-demultiplexing, each barcode region from the given multimeric barcode reagent was isolated from its flanking upstream-adapter and downstream-adapter sequence. Likewise, each molecular sequence identifier region from the given synthetic DNA template molecule was isolated from its flanking upstream and downstream sequences. This process was repeated for each molecule in the sample library; a single filtering step was performed in which individual barcodes and molecular sequence identifiers that were present in only a single read (thus likely to represent either sequencing error or error from the enzymatic sample-preparation process) were censored from the data. For each molecular sequence identifier, the total number of unique (ie with different sequences) barcode regions found associated therewith within single sequence reads was quantitated. A histogram plot was then created to visualize the distribution of this number across all molecular sequence identifiers found in the library.

### DISCUSSION

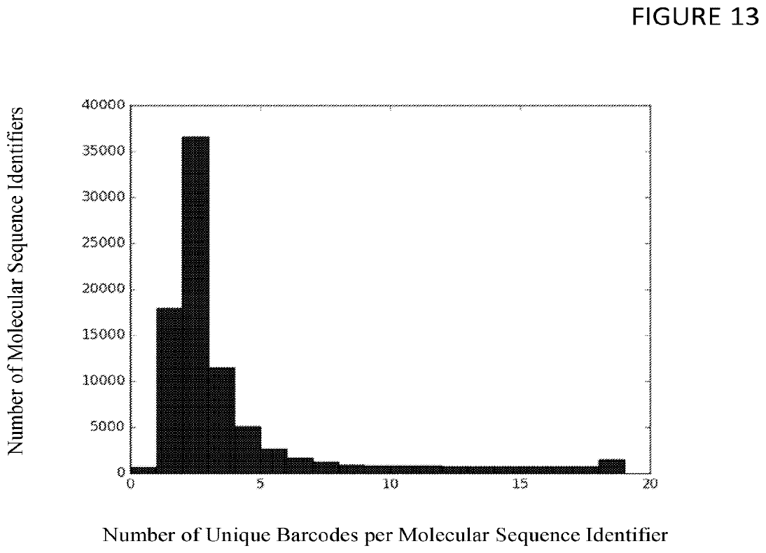

FIG. 13 shows the results of this analysis for Method 6 (Barcoding Synthetic DNA Templates of Known Sequence with Multimeric Barcoding Reagents Containing Barcoded Oligonucleotides). This figure makes clear that the majority of multimeric barcoding reagents are able to successfully label two or more of the tandemly-repeated copies of each molecular sequence identifier with which they are associated. A distribution from 1 to approximately 5 or 6 ‘labelling events’ is observed, indicating that there may be a degree of stochastic interactions that occur with this system, perhaps due to incomplete enzymatic reactions, or steric hindrance at barcode reagent/synthetic template interface, or other factors.

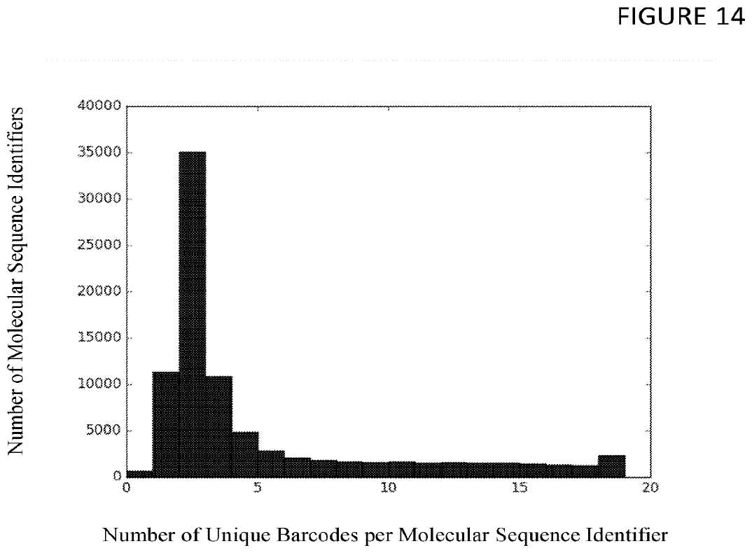

FIG. 14 shows the results of this same analysis conducted using Method 7 (Barcoding Oligonucleotides Synthetic DNA Templates of Known Sequence with Multimeric Barcode Molecules and Separate Adapter Oligonucleotides). This figure also clearly shows that the majority of multimeric barcoding reagents are able to successfully label two or more of the tandemly-repeated copies of each molecular sequence identifier with which they are associated, with a similar distribution to that observed for the previous analysis.

Together, these two figures show that this framework for multimeric molecular barcoding is an effective one, and furthermore that the framework can be configured in different methodologic ways. FIG. 13 shows results based on a method in which the framework is configured such that the multimeric barcode reagents already contain barcoded oligonucleotides, prior to their being contacted with a target (synthetic) DNA template. In contrast, FIG. 14 shows results based on an alternative method in which the adapter oligonucleotides first contact the synthetic DNA template, and then in a subsequent step the adapter oligonucleotides are barcoded through contact with a multimeric barcode reagent. Together these figures demonstrate both the multimeric barcoding ability of these reagents, and their versatility in different key laboratory protocols.

To analyse whether, and the extent to which, individual multimeric barcoding reagents successfully label two or more sub-sequences of the same synthetic DNA template, the groups of different barcodes on each individual multimeric barcoding reagent in the library (as predicted from the Networkx analysis described in the preceding paragraph and as illustrated in FIG. 12) was compared with the barcodes annealed and extended along single synthetic DNA templates (as described in Method 11). Each group of barcodes found on individual multimeric barcoding reagents was given a numeric ‘reagent identifier label’. For each synthetic DNA template molecular sequence identifier (i.e., for each individual synthetic DNA template molecule) that was represented in the sequencing data of Method 11 by two or more barcodes (i.e., wherein two or more sub-sequences of the synthetic template molecule were annealed and extended by a barcoded oligonucleotide), the corresponding ‘reagent identifier label’ was determined. For each such synthetic template molecule, the total number of multimeric barcodes coming from the same, single multimeric barcoding reagent was then calculated (i.e., the number of different sub-sequences in the synthetic template molecule that were labeled by a different barcoded oligonucleotide but from the same, single multimeric barcoding reagent was calculated). This analysis was then repeated and compared with a ‘negative control’ condition, in which the barcodes assigned to each ‘reagent identifier label’ were randomized (i.e. the same barcode sequences remain present in the data, but they no longer correspond to the actual molecular linkage of different barcode sequences across the library of multimeric barcoding reagents).

The data from this analysis is shown in FIG. 17, for both the actual experimental data and for the control data with randomized barcode assignments (note the logarithmic scale of the vertical axis). As this figure shows, though the number of unique barcoding events per target synthetic DNA template molecule is small, they overlap almost perfectly with the known barcode content of individual multimeric barcoding reagents. That is, when compared with the randomized barcode data (which contains essentially no template molecules that appear to be ‘multivalently barcoded’), the overwhelming majority (over 99.9%) of template molecules in the actual experiment that appear to be labeled by multiple barcoded oligonucleotides from the same, individual multimeric barcoding reagent, are in fact labeled multiply by the same, single reagents in solution. By contrast, if there were no non-random association between the different barcodes that labelled individual synthetic DNA templates (that is, if FIG. 17 showed no difference between the actual experimental data and the randomized data), then this would have indicated that the barcoding had not occurred in a spatially-constrained manner as directed by the multimeric barcoding reagents. However, as explained above, the data indicates convincingly that the desired barcoding reactions did occur, in which sub-sequences found on single synthetic DNA templates interacted with (and were then barcoded by) only single, individual multimeric barcoding reagents.

Barcoding Genomic DNA Loci with Multimeric Barcoding Reagents Containing Barcoded Oligonucleotides

### Sequence Extraction and Analysis

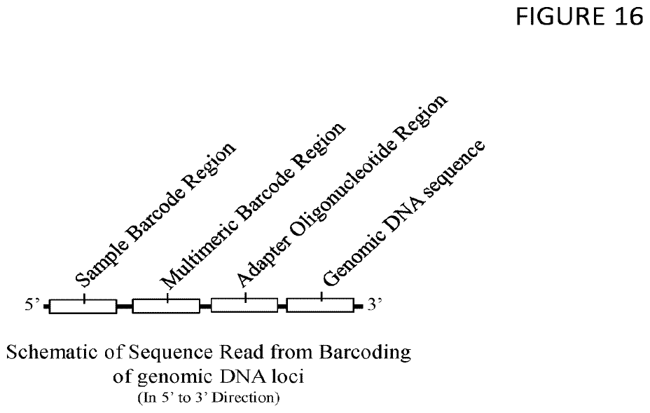

As with other analysis, scripting was composed in Python and implemented in an Amazon Web Services (AWS) framework. For each sequence read following sample-demultiplexing, each barcode region from the given multimeric barcode reagent was isolated from its flanking upstream-adapter and downstream-adapter sequence and recorded independently for further analysis. Likewise, each sequence to the 3′ end of the downstream region (representing sequence containing the barcoded oligonucleotide, and any sequences that the oligonucleotide had primed along during the experimental protocol) was isolated for further analysis. Each downstream sequence of each read was analysed for the presence of expected adapter oligonucleotide sequences (i.e. from the primers corresponding to one of the three genes to which the oligonucleotides were directed) and relevant additional downstream sequences. Each read was then recorded as being either ‘on-target’ (with sequence corresponding to one of the expected, targeted sequence) or ‘off-target’. Furthermore, for each of the targeted regions, the total number of unique multimeric barcodes (i.e. with identical but duplicate barcodes merged into a single-copy representation) was calculated. A schematic of each expected sequence read, and the constituent components thereof, is shown in FIG. 16.

### DISCUSSION

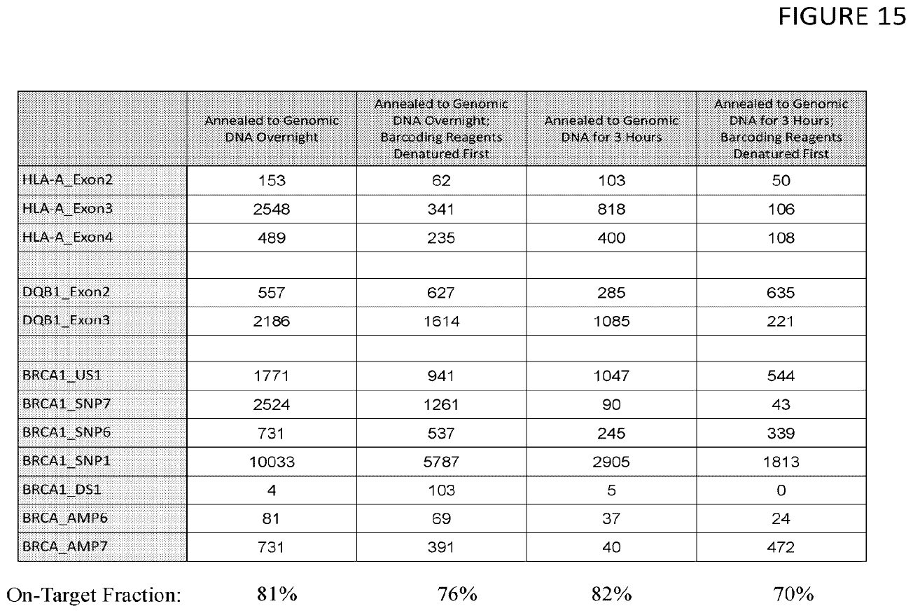

FIG. 15 shows the results of this analysis for this method, for four different independent samples. These four samples represent a method wherein the process of annealing the multimeric barcode reagents took place for either 3 hours, or overnight (approximately 12 hours). Further, for each of these two conditions, the method was performed either with the multimeric barcode reagents retained intact as originally synthesized, or with a modified protocol in which the barcoded oligonucleotides are first denatured away from the barcode molecules themselves (through a high-temperature melting step). Each row represents a different amplicon target as indicated, and each cell represents the total number of unique barcode found associated with each amplicon in each of the four samples. Also listed is the total proportion of on-target reads, across all targets summed together, for each sample.

As seen in the figure, the majority of reads across all samples are on-target; however there is seen a large range in the number of unique barcode molecules observed for each amplicon target. These trends across different amplicons seem to be consistent across the different experimental conditions, and could be due to different priming (or mis-priming) efficiencies of the different oligonucleotides, or different amplification efficiencies, or different mapping efficiencies, plus potential other factors acting independently or in combination. Furthermore, it is clear that the samples that were annealed for longer have a larger number of barcodes observed, likely due to more complete overall annealing of the multimeric reagents to their cognate genomic targets. And furthermore, the samples where the barcoded oligonucleotides were first denatured from the barcode molecules show lower overall numbers of unique barcodes, perhaps owing to an avidity effect wherein fully assembled barcode molecules can more effectively anneal clusters of primers to nearby genomic targets at the same locus. In any case, taken together, this figure illustrates the capacity of multimeric reagents to label genomic DNA molecules, across a large number of molecules simultaneously, and to do so whether the barcoded oligonucleotides remain bound on the multimeric barcoding reagents or whether they have been denatured therefrom and thus potentially able to diffuse more readily in solution.

Method 12—Peptide Coupling of 14.5 μm Carboxy Magnetic Beads with Amino Multimeric hybridization Molecule

1236 μL of Spherotech 14.5 μm (CM-150-10) beads (5824 beads/μL) were placed in a 1.5 mL tube. The beads were pulled on a magnet and the supernatant was removed. The beads were resuspended in 387 μL of 50 mM MES pH 6 buffer, the suspension was then left on a rotator for 10 min. The beads were pulled on a magnet and the supernatant was removed. The beads were resuspended in 387 μL of 50 mM MES pH 6 buffer, the suspension was then left on a rotator for 10 min. The beads were pulled on a magnet and the supernatant was removed. The beads were resuspended in a mixture made of 50 μL of 100 μM oligo (multimeric hybridization molecule, A0147), 11.6 μL of 500 mM MES pH 6 buffer and 54 μL of water. The beads were left on a rotator for 20 min. The beads were then removed from the rotator and then 100 mg of EDC were dissolved in 0.5 mL of 50 mM MES pH 6 buffer. 77 μL of the EDC solution were added to the beads and after mixing the beads were left on a rotator for 2 hours. The beads were then pulled on a magnet and the supernatant was removed. The beads were resuspended in 387 μL of 250 mM Tris pH 8+0.01% Tween20, the suspension was then left on a rotator for 30 min. The beads were then pulled on a magnet and the supernatant was removed. The beads were resuspended in 387 μL of 250 mM Tris pH 8, the suspension was then left on a rotator for 30 min. The beads were then pulled on a magnet and the supernatant was removed. The beads were resuspended in 387 μL of 250 mM Tris pH 8, the suspension was then left on a rotator for 30 min. The beads were then pulled on a magnet and the supernatant was removed. The beads were resuspended in 387 μL of 10 mM Tris pH 8+1 mM EDTA and mixed well. The beads were then pulled on a magnet and the supernatant was removed. The beads were resuspended in 387 μL of 150 mM NaCl, 15 mM Tris pH 7.5, 0.1 mM EDTA and mixed well. The beads were then pulled on a magnet and the supernatant was removed. The beads were resuspended in 387 μL of 150 mM NaCl, 15 mM Tris pH 7.5, 0.1 mM EDTA and mixed well. The final product was stored in the fridge.

Method 13—Attachment of Multimeric Hybridization Molecules to 14.5 μm Carboxy magnetic Beads with Subsequent Extension with an Orthogonal Chemistry
Peptide Coupling of 14.5 μm Carboxy Magnetic Beads with Amino Oligonucleotides (Multimeric Hybridization Molecules)

807 μL of Spherotech 14.5 μm (CM-150-10) beads (5824 beads/μL) were placed in a 1.5 mL tube. The beads were pulled on a magnet and the supernatant was removed. The beads were resuspended in 117 μL of 50 mM MES pH 6 buffer, the suspension was then left on a rotator for min. The beads were pulled on a magnet and the supernatant was removed. The beads were resuspended in 117 μL of 50 mM MES pH 6 buffer, the suspension was then left on a rotator for 10 min. The beads were pulled on a magnet and the supernatant was removed. The beads were resuspended in a mixture made of 12.3 μL of 100 μM oligo (multimeric hybridization molecule, A0120), 3.5 μL of 500 mM MES pH 6 buffer and 19.3 μL of water. The beads were left on a rotator for 20 min. The beads were then removed from the rotator and then 100 mg of EDC were dissolved in 0.5 mL of 50 mM MES pH 6 buffer. 23.5 μL of the EDC solution were added to the beads and after mixing the beads were left on a rotator for 2 hours. The beads were then pulled on a magnet and the supernatant was removed. The beads were resuspended in 117 μL of 250 mM Tris pH 8+0.01% Tween20, the suspension was then left on a rotator for 30 min. The beads were then pulled on a magnet and the supernatant was removed. The beads were resuspended in 117 μL of 250 mM Tris pH 8, the suspension was then left on a rotator for 30 min. The beads were then pulled on a magnet and the supernatant was removed. The beads were resuspended in 117 μL of 250 mM Tris pH 8, the suspension was then left on a rotator for 30 min. The beads were then pulled on a magnet and the supernatant was removed. The beads were resuspended in 117 μL of 10 mM Tris pH 8+1 mM EDTA and mixed well. The beads were then pulled on a magnet and the supernatant was removed. The beads were resuspended in 117 μL of 150 mM NaCl, 15 mM Tris pH 7.5, 0.1 mM EDTA and mixed well. The beads were then pulled on a magnet and the supernatant was removed. The beads were resuspended in 235 μL of 150 mM NaCl, 15 mM Tris pH 7.5, 0.1 mM EDTA and mixed well. The final product was stored in the fridge.

CuAAC Coupling of 14.5 μm Magnetic Beads with Alkyne Multimeric Hybridization Molecules to Azido-Multimeric Hybridization Molecules

25 μL of A0120 beads (20620 beads/μL) were placed in a 1.5 mL tube. The beads were pulled on a magnet and the supernatant was removed. The beads were resuspended in 30 μL of 100 mM HEPES pH 7.0-7.6 buffer and mixed well. The beads were pulled on a magnet and the supernatant was removed. The beads were resuspended in 30 μL of 100 mM HEPES pH 7.0-7.6 buffer and mixed well. The beads were pulled on a magnet and the supernatant was removed. The beads were resuspended in a mixture made of 1.35 μL of 100 μM oligo (A0121), 1.5 μL of 1000 mM HEPES pH 7.0-7.6 buffer and 6.2 μL of water. The beads were mixed well. A solution made of 1.35 μL of 200 μM copper sulphate and 1.89 μL of 1 mM THTPA was prepared and immediately added to the beads. The beads were mixed well. A fresh 1 mM sodium ascorbate solution was prepared and 2.7 μL were added to the beads, mixing well. The tube was sealed in a vacuum bag with an oxygen scavenger bag and it was then left in an incubating rotator at 55° C. for 2 hours. The beads were then pulled on a magnet and the supernatant was removed. The beads were resuspended in 30 μL of 100 mM Tris pH 8+10 mM EDTA, the suspension was then left on a rotator for 10 min. The beads were then pulled on a magnet and the supernatant was removed. The beads were resuspended in 30 μL of 100 mM Tris pH 8+10 mM EDTA, the suspension was then left on a rotator for 10 min. The beads were then pulled on a magnet and the supernatant was removed. The beads were resuspended in 30 μL of 10 mM Tris pH 8+1 mM EDTA and mixed well. The beads were then pulled on a magnet and the supernatant was removed. The beads were resuspended in 30 μL of 150 mM NaCl, 15 mM Tris pH 7.5, 0.1 mM EDTA and mixed well. The beads were then pulled on a magnet and the supernatant was removed. The beads were resuspended in 30 μL of 150 mM NaCl, 15 mM Tris pH 7.5, 0.1 mM EDTA and mixed well. The final product was stored in the fridge.

### Method 14—Assay for Single Cell Whole Transcriptome Sequencing

### Binding of Cells to Multimeric Barcoding Reagents

In a PCR tube, 10 microliters of multimeric barcoding reagents at a concentration of 1,000 reagents/ul are added to the bottom of the tube and incubated for 10 minutes at 4° C. After 10 minutes, 10 microliters of a single cell suspension of cells of interest in PBS (at a concentration of 500 cells/ul) is carefully distributed across the top surface of the reagent layer, without disturbing the settled layer. The tube is then incubated for a further 20 minutes at 4° C. to allow the cells to settle onto the reagent layer.

Dilution of Cell-Reagent Pairs into Lysis and Indexing Solution

After 20 minutes, 30 microlitres of PBS is added to the 20 microlitres of multimeric barcoding reagents and cells, and the whole 50 microlitres is mixed by pipetting up and down 10 times. A 4 microlitre aliquot of the cell-reagent mixture (comprising approximately 250 cells and 500 multimeric barcoding reagents) is transferred to a new PCR tube and 100 microlitres of cold lysis and indexing solution (comprising 100 millimolar Tris-HCl PH 7.5, 10 millimolar EDTA, 150 millimolar LiCl, 0.001% BRIJ58, 30% PEG 4000) is added and mixed by gently pipetting up and down 10 times.

Cell Lysis and Index Annealing to mRNA

The PCR tube containing the cells and multimeric barcoding reagents diluted into lysis and indexing solution is placed on a thermal cycler and incubated at 65° C. for 30 seconds, 45° C. for 1 minute, 35° C. for 3 minutes and then held at 20° C.

mRNA Capture

4 microlitres of mRNA capture reagents are added to the PCR tube and the contents of the tube are mixed by pipetting. The tube is placed on a rotator for 15 minutes at room temperature. After 15 minutes, the tube is placed on a magnet for 10 minutes to allow the magnetic reagents to bind to the magnet. After 10 minutes, the supernatant is discarded. The pellet of reagents is washed with 100 microlitres of a first wash buffer (20 millimolar Tris pH 7.5, 500 millimolar LiCl, 1 millimolar EDTA, 0.1% LiDS), followed by 100 microlitres of a second wash buffer (20 millimolar Tris pH 7.5, 200 millimolar LiCl, 0.5 millimolar EDTA). Each time, the supernatant is discarded.

Reverse Transcription of Captured mRNA and Purification

cDNA of the captured mRNA is generated in the reverse transcription reaction. The washed reagents in the PCR tube are resuspended into a reverse transcription reaction consisting of 2 ul 10×RT buffer (NEB), 2 ul dNTP mix containing 10 mM of each dNTP, 0.25 ul of RiboLock RNase 40 U/ul (ThermoFisher), 0.5 ul of M-MuLV RT enzyme (NEB) and 15.25 ul nuclease-free water.

This reaction is incubated at 42° C. for 1 hour for reverse transcription to take place then 65° C. for 20 mins for deactivation of the MuLV RT enzyme. Due to the RNAse H activity of this enzyme the resulting product will be single stranded cDNA molecule after digestion of the remaining mRNA template.

SPRI (Solid Phase Reversible Immobilization) beads are added to the reaction product at a 1 to 1 ratio of beads to initial reaction volume. The reaction is incubated at room temperature for 5 minutes to bind DNA to beads, the supernatant is removed, then DNA eluted from the beads with addition of 16 μl water and another 5-minute room temperature incubation. After elution 15 μl of the supernatant is removed from the SPRI beads for use in second strand synthesis.

### Second Strand Synthesis and Purification

Second strand synthesis is performed using specific thermocyling conditions and random priming to cover the full length of previously transcribed cDNA molecules. 15 μl of the SPRI-purified single stranded cDNA is added to a master mix containing 2.5 μl 10× thermoPol reaction buffer (NEB), 2.5 μl 10 mM MgSO₄, 0.5 μl 10 mM dNTPs, 1.25 μl of 10 μM A0074 (seq ID number: 8), 0.1 μl of Deep Vent (exo-) (NEB) and 5.15 μl of nuclease-free water to give an overall reaction volume of 25 μl.

The reaction is then heated on a thermocycler with a heated lid (120° C.) beginning with 5 minutes at 95° C., then 3 cycles of: 95° C. for 30 seconds, 4° C. for 3 minutes, 10° C. for 3 minutes, 20° C. for 3 minutes, 30° C. for 3 minutes, 40° C. for 3 minutes, 50° C. for 3 minutes and finally 72° C. for 4 minutes.

The resulting double stranded DNA product is then combined with SPRI beads at a 1 to 1 ratio to the reaction volume. The reaction is incubated at room temperature for 5 minutes to bind DNA to beads, the supernatant is removed, then DNA eluted from the beads with addition of 16 μl water and another 5-minute room temperature incubation. After elution the supernatant is removed from the bead for use in the first polymerase chain reaction (PCR).

### Amplification PCR and Purification

The purified product of the second strand synthesis reaction is amplified in a PCR reaction, 15 μl of the double stranded DNA product is added to a master mix consisting of 2.5 μl of 10× Standard Taq Buffer (NEB), 0.5 μl 10 mM dNTPs, 1.25 μl forward primer A0072 (seq ID number: 9), 1.25 μl reverse primer A0191 (seq ID number: 10), 0.1 μl Hot Start Taq (NEB) and 4.4 μl nuclease-free water to give an overall reaction volume of 25 μl.

The reaction is then heated on a thermocycler with a heated lid (120° C.) beginning with 3 minutes at 95° C. then 30 cycles of: 95° C. for 30 seconds, 58° C. for 15 seconds, 68° C. for 1 minute. Then a final extension step of 68° C. for 2 minutes.

The amplification product is then combined with SPRI beads at a 0.8 to 1 ratio to the reaction volume. The reaction is incubated at room temperature for 5 minutes to bind DNA to beads, the supernatant is removed, washed with ethanol twice, then DNA eluted from the beads with addition of 16 μl water and another 5-minute room temperature incubation. After elution the supernatant is removed from the bead for use in the second (PCR).

### Sequencing Adapter Attachment PCR and Purification

The adapters and sequences required for sample multiplexing are added to each sample in a PCR. 15 μl of the purified PCR product is added to a master mix consisting of 2.5 μl of 10× Standard Taq Buffer (NEB), 0.5 μl 10 mM dNTPs, 0.1 μl Hot Start Taq (NEB) and 5.9 μl nuclease-free water. 1 μl of a 10 μM dilution of a unique index pair is added to each sample to be multiplexed which act as the forward and reverse primers in the PCR.

The reaction is then heated on a thermocycler with a heated lid (120° C.) beginning with 1 minute at 95° C. then 6 cycles of: 95° C. for 30 seconds, 60° C. for 30 seconds, 68° C. for 45 seconds. Then a final extension step of 68° C. for 5 minutes.

The amplification product is then combined with SPRI beads at a 0.6 to 1 ratio to the reaction volume. The reaction is incubated at room temperature for 5 minutes to bind DNA to beads, the supernatant is removed, washed with 80% ethanol twice, then DNA is eluted from the beads with addition of 20 μl water and another 5-minute room temperature incubation. The samples are then ready to be quantified and pooled in preparation for sequencing.

### Method 15

Method 15a—Peptide Coupling of 14.5 μm Carboxy Magnetic Beads with Amino Oligonucleotides (Multimeric Hybridization Molecules)

6868 μL of Spherotech 14.5 μm (CM-150-10) beads (5824 beads/μL) were placed in a 15 mL tube. The beads were pulled on a magnet and the supernatant was removed. The beads were resuspended in 2149 μL of 50 mM MES pH 6 buffer and transferred to a 5 ml tube, the suspension was then left on a rotator for 10 min. The beads were pulled on a magnet and the supernatant was removed. The beads were resuspended in 2149 μL of 50 mM MES pH 6 buffer, the suspension was then left on a rotator for 10 min. The beads were pulled on a magnet and the supernatant was removed. The beads were resuspended in a mixture made of 279 μL of 100 μM oligo (multimeric hybridization molecule, A0147), 64.5 μL of 500 mM MES pH 6 buffer and 301 μL of water. The beads were left on a rotator for 20 min. The beads were then removed from the rotator and then 100 mg of EDC were dissolved in 0.5 mL of 50 mM MES pH 6 buffer. 430 μL of the EDC solution were added to the beads and after mixing the beads were left on a rotator for 2 hours. The beads were then pulled on a magnet and the supernatant was removed. The beads were resuspended in 2149 μL of 250 mM Tris pH 8+0.01% Tween20, the suspension was then left on a rotator for 30 min. The beads were then pulled on a magnet and the supernatant was removed. The beads were resuspended in 2149 μL of 250 mM Tris pH 8, the suspension was then left on a rotator for 30 min. The beads were then pulled on a magnet and the supernatant was removed. The beads were resuspended in 2149 μL of 250 mM Tris pH 8, the suspension was then left on a rotator for 30 min. The beads were then pulled on a magnet and the supernatant was removed. The beads were resuspended in 2149 μL of 10 mM Tris pH 8+1 mM EDTA and mixed well. The beads were then pulled on a magnet and the supernatant was removed. The beads were resuspended in 2149 μL of 150 mM NaCl, 15 mM Tris pH 7.5, 0.1 mM EDTA and mixed well. The beads were then pulled on a magnet and the supernatant was removed. The beads were resuspended in 1800 μL of 150 mM NaCl, 15 mM Tris pH 7.5, 0.1 mM EDTA and mixed well. The final concentration of the beads was found to be 21990 beads/μL. The final product was stored in the fridge.

Method 15b—Barcoded Oligonucleotide Manufacturing Using 2-Step PCR

### Diversity Generation PCR for 1,000 Barcodes Per Pool

A dilution of A0064 (unique oligonucleotides each having a barcode region) is made to a final concentration of 60,221 molecules/ul 0.017 μl of this (1000 molecules) is added to a master mix consisting of 3 μl of 5× HF buffer (NEB), 0.3 μl of 10 mM dNTPs, 0.075 μl of 10 μM of A0065, 0.075 μl of 10 μl of 10 μM A0066, 0.15 μl of 2 U/ul Phusion (NEB) and 11.28 μl of water; this is combined in a PCR tube or PCR plate well. The sample is then subject to 98° C. for 30 seconds, then 33 cycles of 98° C. for 10 seconds, 62° C. for 10 seconds and 72° C. for 15 seconds. There is a final elongation step of 72° C. for 10 seconds. This PCR should amplify ˜1000 different unique barcode sequences which will be further amplified using linear PCR and finally hybridised to a specific quantity of magnetic beads.

### Linear Amplification of the Diversity Generation Reaction

To set up the linear PCR reaction 5 μl from the previous PCR is added to a master mix consisting of 5 μl of 5× HF buffer (NEB), 0.5 μl of 10 mM dNTPs, 0.625 μl of 10 μM A0066, 0.25 ul of 2 U/μl Phusion (NEB) and 13.63 μl of water in a PCR tube. The sample is subject to 98° C. for 30 seconds, then 20 cycles of 98° C. for 10 seconds, 62° C. for 10 seconds and 72° C. for 15 seconds. There is a final elongation step of 72° C. for 10 seconds. This results in amplified product of the previous PCR with heavy bias towards the forward primer product which is the barcoded oligonucleotide of interest which is to be hybridised to the beads. 1 μl of this final reaction volume is set aside for decoding of the individual barcoded oligonucleotide sequences.

Method 15c—Barcoded Oligonucleotide Hybridization to 14.5 μm Magnetic Beads

### Annealing of Barcoded Oligonucleotide to Magnetic Beads

In order to make a library of 3072 different multimeric barcoding reagents, about 12 million beads are necessary from the one prepared according to Method 15a.

The manufactured barcoded oligonucleotides are hybridised to multimeric hybridization molecules on 14.5 μm magnetic beads (prepared according to Method 15a) to create a multimeric barcoding reagent. To do this, 5 μl of the linear amplification product is added to a master mix containing per reaction: 1.25 μl of 80% Polyethylene glycol (PEG) 4000 solution, 1 μl 5M Sodium Chloride, 0.15 μl 1M Tris-HCl PH 7.5, 0.002 μl 500 mM Ethylenediaminetetraacetic acid (EDTA), 0.145 μl 21,990 beads/μl 14.5 μm multimeric hybridization molecule conjugated magnetic beads (prepared according to Method 15a), 0.025 μl 100 μM palmitate conjugated oligo 10 μM A0032, 0.1 μl 1M MgCl2 and 2.328 μl nuclease-free water. This is then heated in a thermocycler to 70° C. for 1 minute then the temperature will reduce by 1° C. and hold for a further minute. This reduction of heat and holding will continue until the temperature falls to 10° C. After the final 1-minute hold, the temperature will drop to 4° C. and hold indefinitely.

### Quenching, Pooling and Washing of Multimeric Barcoding Reagents

After completion of hybridization of the multimeric barcoding reagents to magnetic beads, the hybridization needs to be quenched to inhibit jumping of barcoded oligonucleotides to undesired beads. This would have a negative impact on the sensitivity of the intended use of the reagents. To begin quenching, 10 μl of an excess of A0187 (a blocking oligonucleotide that is used to quench the hybridization reaction preventing the hybridization of barcoded oligonucleotides to different beads during the reagent-pooling reaction), relative to the input concentration of barcoded oligonucleotide in the hybridization reaction, is added to the hybridization reaction at room temperature. All 20 μl of each quenched hybridization reaction is pooled into one tube and the beads are pulled to a magnet. The supernatant is removed and replaced with an excess of PBS with 3 mM MgCl₂. The beads are kept on magnet and washed a further two times with PBS and MgCl₂ and finally resuspended in a 48^{th} of the initial volume to allow use of the library at sensible concentrations, for example for 9.6 ml of reaction volume washed the resuspension volume would be 200 μl.

Method 15d—Decoding of Multimeric Barcoding Reagents

In order to use the Multimeric Barcoding Reagents in a successful single-cell sequencing experiment it is important the Barcode of each reagent is known. A 1 μl aliquot from taken from the linear amplification step is added to a master mix consisting of: 1 μl standard Taq reaction buffer (NEB), 0.2 μl 10 mM dNTPs, 0.04 μl of 5 U/μl Hot Start Taq, 0.07 μl 1 μM A0105, 5.69 μl nuclease-free water and 2.5 μl of a pair of unique index Illumina® sequencing adapters at 2.5 μM. The reaction is heated in a thermocycler for 95° C. for 1 minute, 50° C. for 30 seconds, 68° C. for 20 seconds then 13 cycles of 95° C. for 15 seconds and 68° C. for 20 seconds. There is a final elongation step of 68° C. for 30 seconds. Once completed all the decoding reactions are pooled, washed using SPRI beads with two 80% ethanol washes and eluted in a volume between 100 μl to 500 μl water depending on the intended library size. This library is then able to be quantified and diluted appropriately for the platform intended for sequencing of the pool.

Method 15e—Assay for Single Cell Whole Transcriptome Sequencing

### Binding of Cells to Multimeric Barcoding Reagents

In a PCR tube, 10 microliters of multimeric barcoding reagents (prepared according to Method 15c) at a concentration of 1,000 reagents/ul are added to the bottom of the tube and incubated for 10 minutes at 4° C. After 10 minutes, 10 microliters of a single cell suspension of cells of interest in PBS (at a concentration of 500 cells/ul) is carefully distributed across the top surface of the reagent layer, without disturbing the settled layer. The tube is then incubated for a further 20 minutes at 4° C. to allow the cells to settle onto the reagent layer.

Dilution of Cell-Reagent Pairs into Lysis and Indexing Solution

After 20 minutes, 30 microlitres of PBS is added to the 20 microlitres of multimeric barcoding reagents and cells, and the whole 50 microlitres is mixed by pipetting up and down 10 times. A 4 microlitre aliquot of the cell-reagent mixture (comprising approximately 250 cells and 500 multimeric barcoding reagents) is transferred to a new PCR tube and 100 microlitres of cold lysis and indexing solution (comprising 100 millimolar Tris-HCl PH 7.5, 10 millimolar EDTA, 150 millimolar LiCl, 0.001% BRIJ58, 30% PEG 4000) is added and mixed by gently pipetting up and down 10 times.

Cell Lysis and Index Annealing to mRNA

The PCR tube containing the cells and multimeric barcoding reagents diluted into lysis and indexing solution is placed on a thermal cycler and incubated at 65° C. for 30 seconds, 45° C. for 1 minute, 35° C. for 3 minutes and then held at 20° C.

mRNA Capture

4 microlitres of mRNA capture reagents are added to the PCR tube and the contents of the tube are mixed by pipetting. The tube is placed on a rotator for 15 minutes at room temperature. After 15 minutes, the tube is placed on a magnet for 10 minutes to allow the magnetic reagents to bind to the magnet. After 10 minutes, the supernatant is discarded. The pellet of reagents is washed with 100 microlitres of a first wash buffer (20 millimolar Tris pH 7.5, 500 millimolar LiCl, 1 millimolar EDTA, 0.1% LiDS), followed by 100 microlitres of a second wash buffer (20 millimolar Tris pH 7.5, 200 millimolar LiCl, 0.5 millimolar EDTA). Each time, the supernatant is discarded.

Reverse Transcription of Captured mRNA and Purification

cDNA of the captured mRNA is generated in the reverse transcription reaction. The washed reagents in the PCR tube are resuspended into a reverse transcription reaction consisting of 2 μl 10×RT buffer (NEB), 2 μl dNTP mix containing 10 mM of each dNTP, 0.25 μl of RiboLock RNase 40 U/μl (ThermoFisher), 0.5 μl of M-MuLV RT enzyme (NEB) and 15.25 μl nuclease-free water.

This reaction is incubated at 42° C. for 1 hour for reverse transcription to take place then 65° C. for 20 mins for deactivation of the MuLV RT enzyme. Due to the RNAse H activity of this enzyme the resulting product will be single stranded cDNA molecule after digestion of the remaining mRNA template.

SPRI (Solid Phase Reversible Immobilization) beads are added to the reaction product at a 1 to 1 ratio of beads to initial reaction volume. The reaction is incubated at room temperature for 5 minutes to bind DNA to beads, the supernatant is removed, then DNA eluted from the beads with addition of 16 μl water and another 5-minute room temperature incubation. After elution 15 μl of the supernatant is removed from the SPRI beads for use in second strand synthesis.

### Second Strand Synthesis and Purification

Second strand synthesis is performed using specific thermocyling conditions and random priming to cover the full length of previously transcribed cDNA molecules. 15 μl of the SPRI-purified single stranded cDNA is added to a master mix containing 2.5 μl 10× thermoPol reaction buffer (NEB), 2.5 μl 10 mM MgSO₄, 0.5 μl 10 mM dNTPs, 1.25 μl of 10 μM A0074 (seq ID number: 8), 0.1 μl of Deep Vent (exo-) (NEB) and 5.15 μl of nuclease-free water to give an overall reaction volume of 25 μl.

The reaction is then heated on a thermocycler with a heated lid (120° C.) beginning with 5 minutes at 95° C., then 3 cycles of: 95° C. for 30 seconds, 4° C. for 3 minutes, 10° C. for 3 minutes, 20° C. for 3 minutes, 30° C. for 3 minutes, 40° C. for 3 minutes, 50° C. for 3 minutes and finally 72° C. for 4 minutes.

The resulting double stranded DNA product is then combined with SPRI beads at a 1 to 1 ratio to the reaction volume. The reaction is incubated at room temperature for 5 minutes to bind DNA to beads, the supernatant is removed, then DNA eluted from the beads with addition of 16 μl water and another 5-minute room temperature incubation. After elution the supernatant is removed from the bead for use in the first polymerase chain reaction (PCR).

### Amplification PCR and Purification

The purified product of the second strand synthesis reaction is amplified in a PCR reaction, 15 μl of the double stranded DNA product is added to a master mix consisting of 2.5 μl of 10× Standard Taq Buffer (NEB), 0.5 μl 10 mM dNTPs, 1.25 μl forward primer A0072 (seq ID number: 9), 1.25 μl reverse primer A0191 (seq ID number: 10), 0.1 μl Hot Start Taq (NEB) and 4.4 μl nuclease-free water to give an overall reaction volume of 25 μl.

The reaction is then heated on a thermocycler with a heated lid (120° C.) beginning with 3 minutes at 95° C. then 30 cycles of: 95° C. for 30 seconds, 58° C. for 15 seconds, 68° C. for 1 minute. Then a final extension step of 68° C. for 2 minutes.

The amplification product is then combined with SPRI beads at a 0.8 to 1 ratio to the reaction volume. The reaction is incubated at room temperature for 5 minutes to bind DNA to beads, the supernatant is removed, washed with ethanol twice, then DNA eluted from the beads with addition of 16 μl water and another 5-minute room temperature incubation. After elution the supernatant is removed from the bead for use in the second (PCR).

### Sequencing Adapter Attachment PCR and Purification

The adapters and sequences required for sample multiplexing are added to each sample in a PCR. 15 μl of the purified PCR product is added to a master mix consisting of 2.5 μl of 10× Standard Taq Buffer (NEB), 0.5 μl 10 mM dNTPs, 0.1 μl Hot Start Taq (NEB) and 5.9 μl nuclease-free water. 1 μl of a 10 μM dilution of a unique index pair is added to each sample to be multiplexed which act as the forward and reverse primers in the PCR.

The reaction is then heated on a thermocycler with a heated lid (120° C.) beginning with 1 minute at 95° C. then 6 cycles of: 95° C. for 30 seconds, 60° C. for 30 seconds, 68° C. for 45 seconds. Then a final extension step of 68° C. for 5 minutes.

The amplification product is then combined with SPRI beads at a 0.6 to 1 ratio to the reaction volume. The reaction is incubated at room temperature for 5 minutes to bind DNA to beads, the supernatant is removed, washed with 80% ethanol twice, then DNA is eluted from the beads with addition of 20 μl water and another 5-minute room temperature incubation. The samples are then ready to be quantified and pooled in preparation for sequencing.

### Method 16—Multimeric Barcoding Reagent Barcoded Oligonucleotide Quantification Assay

Summary—This protocol allows approximate quantification of the number of barcoded oligonucleotides on a library bead. This method utilises 5 μL qPCR reactions and compares to a standard for quantification.

Ensure you have an accurate quantification of the bead concentration before starting the assay.

The oligos required for the assay change depending on the barcoded oligonucleotide structure used. Ensure the correct oligonucleotides are selected for the barcoded oligonucleotide type on your bead of interest:

### 16NIO Barcoded Oligonucleotide Testing Requirements

Oligonucleotides are required at the following concentrations, Forward Primer (A0067) at 10 μM, Reverse Primer (A0055) at 10 μM, Standard Primer (A0071) at 5 μM and Template Oligonucleotide (A0068) at 500 nM.

### SynIO Barcoded Oligonucleotide Testing Requirements

Oligonucleotides are required at the following concentrations, Forward Primer (A0127) at 10 μM, Reverse Primer (A0128) at 10 μM, Standard Primer (A0125) at 5 μM and Template Oligonucleotide (A0126) at 500 nM.

### Requirements of Assay:

Nuclease free water, Luna® Universal qPCR Master Mix (NEB), Library of multimeric barcoding reagents, qPCR consumables (tubes, caps, plates or seals), and Standard oligonucleotide (standard for calibration), Fw Primer, Rv Primer and Template Oligonucleotides.

### Prepare Calibration Curve:

Prepare the calibration curve using the 5 μM starting oligonucleotide stock. A serial dilution is performed from samples 1-7. Mix tubes well after addition of each oligonucleotide during the serial dilution. From the 5 μM standard primer prepare a 0.3 μM concentration. From this prepare 6 subsequent dilutions using a 10-fold serial dilution methodology. The 8^{th} tube should just contain water as a negative control. Seal the strip (Containing 1-7 and NEG) until required.

### Prepare Samples:

Set a PCR machine or tube incubator to 95° C. for an infinite amount of time. Dilute beads to 10 b/μL in nuclease free water to 100 μL. Ensure the beads are mixed and homogenous in the tube before sampling or else the quantification will be incorrect. Mix the 10 b/μL samples again, seal and quickly move to the PCR machine for 60 s. After 60 s, remove the samples from the incubator and place on a magnet. Leave for 2 minutes. After 2 minutes, transfer 50 μL of the solution to another tube on a magnet (this step depletes any bead carryover). Dilute the sample in water further to the desired bead equivalence. Ensure mixing is performed during dilutions required. For 15 μm beads recommend 1b/ul eq. For 30-100 μm beads recommend 0.1 b/μl eq.

Prepare qPCR Master Mix and Add Samples:

Scale the following reaction depending on the number of wells required in the assay (+15% spare). For a 5 μl Reaction combine 2.5 μl of Luna Universal qPCR Master Mix and 0.125 μls of the Forward Primer, Reverse Primer and Template DNA. Scale the reaction appropriately for the number of samples. Mix the master mix with a pipette well.

Distribute 2.875 μL to each required well of the assay plate. Add 2.125 μL of the sample/calibration to appropriate well. Seal qPCR plate/strips and place onto machine.

qPCR Protocol-Quant Studio 1-applied biosystems (Thermo Fisher Scientific)
95° C. for 30 s
45° C. for 20 s
68° C. for 8 s
   35×
(
95° C. for 30 s
58° C. for 10 s
68° C. for 8 s
)
68° C. for 300 s

### Results and Analysis

Determine the concentration of barcoded oligonucleotide according to the standard curve. Adjust results depending on the input concentration to the qPCR and the initial concentration of the beads at the point of oligonucleotide dehybridisation. Determine the number of barcoded oligonucleotides approximately hybridised to each bead.

### Method 17—Solution Barcoded Oligonucleotide Quantification Assay

Summary—This protocol allows approximate quantification of the number of barcoded oligonucleotides in a volume of solution. This method utilises 5 μL qPCR reactions and compares to a standard for quantification.

The oligos required for the assay change depending on the barcoded oligonucleotide structure used. Ensure the correct oligonucleotides are selected for the barcoded oligonucleotide of interest:

### 16NIO Barcoded Oligonucleotide Testing Requirements

Oligonucleotides are required at the following concentrations, Forward Primer (A0067) at 10 μM, Reverse Primer (A0055) at 10 μM, Standard Primer (A0071) at 5 μM and Template Oligonucleotide (A0068) at 500 nM.

### SynIO Barcoded Oligonucleotide Testing Requirements

Oligonucleotides are required at the following concentrations, Forward Primer (A0127) at 10 μM, Reverse Primer (A0128) at 10 μM, Standard Primer (A0125) at 5 μM and Template Oligonucleotide (A0126) at 500 nM.

### Requirements of Assay:

Nuclease free water, Luna® Universal qPCR Master Mix (NEB), Library of multimeric barcoding reagents, qPCR consumables (tubes, caps, plates or seals), and Standard oligonucleotide (standard for calibration), Fw Primer, Rv Primer and Template Oligonucleotides.

### Prepare Calibration Curve:

Prepare the calibration curve using the 5 μM starting oligonucleotide stock. A serial dilution is performed from samples 1-7. Mix tubes well after addition of each oligonucleotide during the serial dilution. From the 5 μM standard primer prepare a 0.3 μM concentration. From this prepare 6 subsequent dilutions using a 10-fold serial dilution methodology. The 8th tube should just contain water as a negative control. Seal the strip (Containing 1-7 and NEG) until required.

### Prepare Samples:

Dilute samples to a concentration that would be quantifiable within the assay, or if this in unknown perform a serial dilution of the sample so that a suitable concentration may be found in the results of the assay. Aim for 100 μl samples. Place samples on a magnet. Leave for 2 minutes. After 2 minutes, transfer 50 μL of the solution to another tube on a magnet (this step depletes any potential bead carryover). Dilute the sample in water further to the desired concentration. Ensure mixing is performed during dilutions required.

Prepare qPCR Master Mix and Add Samples:

Scale the following reaction depending on the number of wells required in the assay (+15% spare). For a 5 μl Reaction combine 2.5 μl of Luna Universal qPCR Master Mix and 0.125 μls of the Forward Primer, Reverse Primer and Template DNA. Scale the reaction appropriately for the number of samples. Mix the master mix with a pipette well. Distribute 2.875 UL to each required well of the assay plate. Add 2.125 μL of the sample/calibration to appropriate well. Seal qPCR plate/strips and place onto machine.

qPCR Protocol—Quant Studio 1—Applied Biosystems (Thermo Fisher Scientific)
95° C. for 30 s
45° C. for 20 s
68° C. for 8 s
   35×
(
95° C. for 30 s
58° C. for 10 s
68° C. for 8 s
)
68° C. for 300 s

### Results and Analysis

Determine the concentration of barcoded oligonucleotide according to the standard curve. Adjust results depending on the input concentration to the qPCR.

### Method 18—Assay for Single Cell Whole Transcriptome Sequencing

### Binding of Cells to Multimeric Barcoding Reagents

In a PCR tube, 10 microliters of multimeric barcoding reagents at a concentration of 1,000 reagents/ul are added to the bottom of the tube and incubated for 10 minutes at 4° C. After 10 minutes, 10 microliters of a single cell suspension of cells of interest in PBS (at a concentration of 500 cells/ul) is carefully distributed across the top surface of the reagent layer, without disturbing the settled layer. The tube is then incubated for a further 20 minutes at 4° C. to allow the cells to settle onto the reagent layer.

Dilution of Cell-Reagent Pairs into Lysis and Indexing Solution and Flash-Freezing

After 20 minutes, the whole 20 microlitres is mixed by pipetting up and down 10 times. A 4 microlitre aliquot of the cell-reagent mixture (comprising approximately 1000 cells and 2000 multimeric barcoding reagents) is transferred to a new PCR tube and 100 microlitres of cold lysis and indexing solution (comprising 100 millimolar Tris-HCl PH 7.5, 10 millimolar EDTA, 150 millimolar LiCl, 0.001% BRIJ58, 30% PEG 4000, 20% y-butyrolactone and 4 microlitres of mRNA capture reagents) is added and mixed by gently pipetting up and down 10 times. Immediately after mixing, the tube is flash-frozen in dry ice for 15 minutes.

Cell Lysis, mRNA Barcoding and Capture

After 15 minutes in dry ice, the PCR tube containing the cells and multimeric barcoding reagents in lysis and indexing solution is placed on a thermal cycler and incubated at 65° C. for 30 seconds, followed by a stepwise gradient of 55° C. to 40° C. for 90 seconds, with −1° C. every 6 seconds, followed by another stepwise gradient of 35° C. to 20° C. for 90 seconds with −1° C. every 9 seconds, and then held at 20° C. The mixture is diluted by the addition of 100 μl of capture dilution buffer (comprising 100 millimolar Tris-HCl PH 7.5, 10 millimolar EDTA, 150 millimolar LiCl) and the tube is placed on a rotator for 5 minutes at room temperature to allow further capture of mRNA on the mRNA capture reagents). After 5 minutes, the tube is placed on a magnet for 10 minutes to allow the magnetic reagents to bind to the magnet. After 10 minutes, the supernatant is discarded. The pellet of reagents is washed with 100 microlitres of a first wash buffer (20 millimolar Tris pH 7.5, 500 millimolar LiCl, 1 millimolar EDTA, 0.1% LiDS), followed by 100 microlitres of a second wash buffer (20 millimolar Tris pH 7.5, 200 millimolar LiCl, 0.5 millimolar EDTA). Each time, the supernatant is discarded.

Reverse Transcription of Captured mRNA and Purification

cDNA of the captured mRNA is generated in the reverse transcription reaction. The washed reagents in the PCR tube are resuspended into a reverse transcription reaction consisting of 2 μl 10×RT buffer (NEB), 2 μl dNTP mix containing 10 mM of each dNTP, 0.25 μl of RiboLock RNase 40 U/μl (ThermoFisher), 2 μl of M-MuLV RT enzyme (NEB), 2.5% PEG 2000 (2.5 μl of a 20% stock) and 11.25 μl nuclease-free water.

This reaction is incubated at 42° C. for 1 hour for reverse transcription to take place then 65° C. for 20 mins for deactivation of the MuLV RT enzyme, followed by an incubation at 95° C. for 30 seconds to increase the release of single stranded cDNA into solution.

SPRI (Solid Phase Reversible Immobilization) beads are added to the reaction product at a 1 to 1 ratio of beads to initial reaction volume. The reaction is incubated at room temperature for 5 minutes to bind DNA to beads, the supernatant is removed, then DNA eluted from the beads with addition of 16 μl water and another 5-minute room temperature incubation. After elution 15 μl of the supernatant is removed from the SPRI beads for use in second strand synthesis.

### Second Strand Synthesis and Purification

Second strand synthesis is performed using specific thermocyling conditions and random priming to cover the full length of previously transcribed cDNA molecules. 15 μl of the SPRI-purified single stranded cDNA is added to a master mix containing 2.5 μl 10× thermoPol reaction buffer (NEB), 2.5 μl 10 mM MgSO₄, 0.5 μl 10 mM dNTPs, 1.25 μl of 10 μM A0074 (seq ID number: 8), 0.5 μl of Deep Vent (exo-) (NEB), 3% of PEG 20000 (2.14 μl of a 35% stock) and 2.46 μl of nuclease-free water to give an overall reaction volume of 25 μl.

The reaction is then heated on a thermocycler with a heated lid (120° C.) beginning with 5 minutes at 95° C., then 3 cycles of: 95° C. for 30 seconds, 30° C. for 3 minutes, 40° C. for 3 minutes, 50° C. for 3 minutes and 72° C. for 4 minutes, before being held at 4° C.

The resulting double stranded DNA product is then combined with SPRI beads at a 1 to 1 ratio to the reaction volume. The reaction is incubated at room temperature for 5 minutes to bind DNA to beads, the supernatant is removed, then DNA eluted from the beads with addition of 16 μl water and another 5-minute room temperature incubation. After elution the supernatant is removed from the bead for use in the first polymerase chain reaction (PCR).

### Amplification PCR and Purification

The purified product of the second strand synthesis reaction is amplified in a PCR reaction, 15 μl of the double stranded DNA product is added to a master mix consisting of 2.5 μl of 10× Standard Taq Buffer (NEB), 0.5 μl 10 mM dNTPs, 1.25 μl forward primer A0072 (seq ID number: 9), 1.25 μl reverse primer A0191 (seq ID number: 10), 0.1 μl Hot Start Taq (NEB) and 4.4 μl nuclease-free water to give an overall reaction volume of 25 μl.

The reaction is then heated on a thermocycler with a heated lid (120° C.) beginning with 3 minutes at 95° C. then 15 cycles of: 95° C. for 30 seconds, 58° C. for 15 seconds, 68° C. for 1 minute. Then a final extension step of 68° C. for 2 minutes.

The amplification product is then combined with SPRI beads at a 0.8 to 1 ratio to the reaction volume. The reaction is incubated at room temperature for 5 minutes to bind DNA to beads, the supernatant is removed, washed with ethanol twice, then DNA eluted from the beads with addition of 16 μl water and another 5-minute room temperature incubation. After elution the supernatant is removed from the bead for use in the second PCR.

### Sequencing Adapter Attachment PCR and Purification

The adapters and sequences required for sample multiplexing are added to each sample in a PCR. 15 μl of the purified PCR product is added to a master mix consisting of 2.5 μl of 10× Standard Taq Buffer (NEB), 0.5 μl 10 mM dNTPs, 0.1 μl Hot Start Taq (NEB) and 5.9 μl nuclease-free water. 1 μl of a 10 μM dilution of a unique index pair is added to each sample to be multiplexed which act as the forward and reverse primers in the PCR.

The reaction is then heated on a thermocycler with a heated lid (120° C.) beginning with 1 minute at 95° C. then 6 cycles of: 95° C. for 30 seconds, 60° C. for 30 seconds, 68° C. for 45 seconds. Then a final extension step of 68° C. for 5 minutes.

The amplification product is then combined with SPRI beads at a 0.6 to 1 ratio to the reaction volume. The reaction is incubated at room temperature for 5 minutes to bind DNA to beads, the supernatant is removed, washed with 80% ethanol twice, then DNA is eluted from the beads with addition of 20 μl water and another 5-minute room temperature incubation. The samples are then ready to be quantified and pooled in preparation for sequencing.

TABLE 1
Selected sequences and their corresponding SEQ ID NOs.
All sequences in the table are DNA sequences. Sequences represented by multiple SEQ ID NOs
are listed in the sequence listing by presenting amino acid sequences on either side of the internal
spacer region (represented by X as defined in the “additional information” column). The indicator
“X” followed by a number in parentheses in the “additional information” column describes
modification at a location identified by the number in parentheses. For example, “X(35) = 3′Inverted
dT” means that the residue at location 35 in the sequence is 3′Inverted dT.
OLIGONUCLEOTIDE SEQUENCES

| ID | Sequence | Additional information |
|---|---|---|
| A0074 | TACACGACGCTCTTCCGATCTNNNNNNN | |
| A0072 | ACGACGCTCTTCCGATCT | |
| A0191 | GTGACTGGAGTTCAGACGTG | |
| A0064 | GTGACTGGAGTTCAGACGTGTGCTCTTCCGATCTNNNNN | |
| | NNNNNNNNNNNCGTGCTTTTTTTTTTTTTTTTTTTTTTTTT | |
| A0187 | AGATCGGAAGAGCACACGTCTGAACTCCAGTCACX | X(35) = 3′ Inverted dT |
| A0113 | XGTGACTGGAGTTCAGACG | X(1) = Phosphorothioate bond |
| A0115 | XAAAAAAAAAAAAAAAAAAAAAAAAAGCACG | X(1) = 5′ Phosphate |
| A0066 | GTGACTGGAGTTCAGACG | |
| A0065 | AAAAAAAAAAAAAAAAAAAAAAAAAGCACG | |
| A0105 | ACACTCTTTCCCTACACGACGCTCTTCCGATCTNNNNNNN | X(90) = 3′ Inverted dT |
| | NNNNNNNNNNNNNNNNNNNAAAAAAAAAAAAAAAAAAA | |
| | AAAAAAGCACGX | |
| A0147 | CGTGACCAGCGACTCAGCCTTTXTTTAGATCGGAAGAGC | X(23) = IDT internal spacer 18; X(45) = IDT internal |
| | ACTTTXTTTAGATCGGAAGAGCACTTTXTTTAGATCGGAAG | spacer 18; X(67) = IDT internal spacer 18; X(86) = IDT |
| | AGCACXXX | internal spacer 18; X(87) = IDT intemal spacer 18; |
| | | X(88) = IDT 3′ amino modifier |
| A0120 | XXTTTAGATCGGAAGAGCACTTTXTTTAGATCGGAAGAGC | spacer 18; X(68) = IDT internal spacer 18; X(87) = IDT |
| | ACTTTXTTTAGATCGGAAGAGCACTTTXTTTAGATCGGAAG | X(1) = 5′ hexynyl; X(2) = IDT internal spacer 18; |
| | AGCACXXX | X(24) = IDT internal spacer 18; X(46) = IDT internal |
| | | internal spacer 18; X(88) = IDT internal spacer 18; |
| | | X(89) = IDT 3′ amino modifier |
| A0121 | CGTGACCAGCGACTCAGCCTTXTTTAGATCGGAAGAGCA | X(22) = IDT internal spacer 18; X(44) = IDT internal |
| | CTTTXTTTAGATCGGAAGAGCACTTTXTTAGATCGGAAGA | spacer 18; X(66) = IDT internal spacer 18; X(84) = IDT |
| | GCACXX | internal spacer 18; X(85) = 3′ azide or 3′ amino |
| | | coupled with azide-NHS-ester |
| A0148 | CGTGACCAGCGACTCAGCCTTTXTTTGATCGGAAGAGCA | X(23) = IDT internal spacer 18; X(44) = IDT internal |
| | CTTTXTTTGATCGGAAGAGCACTTTXTTTGATCGGAAGAGC | spacer 18; X(65) = IDT internal spacer 18; X(83) = IDT |
| | ACXXX | internal spacer 18; X(84) = IDT internal spacer 18; |
| | | X(85) = IDT 3′ amino modifier |
| A0149 | CGTGACCAGCGACTCAGCCTTTXTTTAGATCGGAAGAGC | X(23) = IDT internal spacer 18; X(44) = IDT internal |
| | ATTTXTTTAGATCGGAAGAGCATTTXTTTAGATCGGAAGAG | spacer 18; X(65) = IDT internal spacer 18; X(83)= IDT |
| | CAXXX | intemal spacer 18; X(84) = IDT internal spacer 18; |
| | | X(85) = IDT 3′ amino modifier |
| A0150 | CGTGACCAGCGACTCAGCCTTTXTTTGATCGGAAGAGCA | X(23) = IDT internal spacer 18; X(43) = IDT internal |
| | TTTXTTTGATCGGAAGAGCATTTXTTTGATCGGAAGAGCAX | spacer 18; X(63) = IDT internal spacer 18; X(80) = IDT |
| | XX | internal spacer 18; X(81) = IDT internal spacer 18; |
| | | X(82) = IDT 3′ amino modifier |
| A0175 | CGTGACCAGCGACTCAGCCTTTXTTTAGATCGGAAGAGC | X(23) = IDT internal spacer 18; X(51) = IDT internal |
| | ACACGTCTTTTXTTTAGATCGGAAGAGCACACGTCTTTTXT | spacer 18; X(79) = IDT internal spacer 18; X(104) = |
| | TTAGATCGGAAGAGCACACGTCTXXX | IDT intemal spacer 18; X(105) = IDT internal spacer |
| | | 18; X(106) = IDT 3′ amino modifier |
| A0177 | CGTGACCAGCGACTCAGCCTTTXTTT | X(23) = IDT internal spacer 18; X(45) = IDT internal |
| | XTTTAGATCGGAAGAGCACTTTXTTTAGATCGGAAGAGCA | spacer 18; X(67) = IDT internal spacer 18; X(86) = IDT |
| | CXXX | internal spacer 18; X(87) = IDT internal spacer 18; |
| | | X(88) = IDT 3′ amino modifier |
| A0180 | CGTGACCAGCGACTCAGCCTTTTTTAGATCGGAAGAGCA | X(89) = IDT 3′ amino modifier |
| | CTTTTTTAGATCGGAAGAGCACTTTTTTAGATCGGAAGAG | |
| | CACTTTTTTX | |
| A0032 | GGCTGAGTCGCTGGTCACGXX | X(20) = Spacer-CE Phosphoramidite 18 (HEG); |
| | | X(21) = 3′-Palmitate SynBase™ CPG 1000/110 |
| A0119 | GTGACTGGAGTTCAGACGTGTGCTCTTCCGATCTGTAGTG | |
| | ATTCCAATTACGTGCTTTTTTTTTTTTTTTTTTTTTTTTT | |
| A0129 | GGAGTTCAGACGTGTGCTCTTCCGATCTTTTTTAATTTTTTT | |
| | TTTTTTTTTTTTTTTTTT | |
| A0184 | ATCGGAAGAGCX | X(12) = 3′ inverted dT |
| A0185 | AGATCGGAAGAGCACX | X(16) = 3′ inverted dT |
| A0186 | AGATCGGAAGAGCACACGTCTGAACX | X(26) = 3′ inverted dT |
| A0067 | GTGCAGTTGGGACGCT | |
| A0068 | GTGCAGTTGGGACGCTGAAGTAACGCAACTTATGAGGA | X(100) = 3′ inverted dT |
| | AGGCTAATATAGCAGGAACTAAATTCCAGTGAAAAAAAA | |
| | AAAAAAAAAAAAAAAAAAGATCX | |
| A0055 | GTGACTGGAGTTCAGACGTG | |
| A0071 | GTGACTGGAGTTCAGACGTGTGCTCTTCCGATCTNNNNN | |
| | NNNNNNNNNNNCGTGCTTTTTTTTTTTTTTTTTTTTTTTTT | |
| A0125 | GGAGTTCAGACGTGTGCTCTTCCGATCTTTTTTAATTTTTTT | |
| A0126 | GTGCAGTTGGGACGCTGAAGTAACGCAACTTATGAGGA | X(96) = 3′ inverted dT |
| | AGGCTAATATAGCAGGAACTAAATTCCAGTGAAAAAAAA | |
| | AAAAAAAAAAAAAAAAATX | |
| A0127 | GTGCAGTTGGGACGCT | |
| A0128 | GGAGTTCAGACGTGTGCTC | |

| | | | SEQ ID |
|---|---|---|---|
| ID | Name | Comments | NO: |
| A0074 | A0074-P5_Amp_N7_L1 | MALBAC primer | 317 |
| A0072 | A0072-P5-Amp_L2 | Forward Amp | 318 |
| | | PCR primer | |
| A0191 | A0191-Illumina_R2_R1_L1 | Reverse Amp | 319 |
| | | PCR primer | |
| A0064 | 101_16N | Template for | 320 |
| | | diversity | |
| | | generation PCR | |
| A0187 | Hyb_Block_4 | Hyb. Block Oligo | 321 |
| A0113 | prFw_IO_prep_pt | Forward primer IO | 322 |
| | | MFG Pt | |
| A0115 | prRv_IO_prep_p | Reverse primer IO | 323 |
| | | MFG P | |
| A0066 | prFw_IO_prep | Forward primer IO | 324 |
| | | MFG | |
| A0065 | prRv_IO_prep | Reverse primer IO | 325 |
| | | MFG | |
| A0105 | DO2_dir | Decoding | 326 |
| | | template | |
| A0147 | A0147-CS5_U_NH2 | | 327, 328, |
| | | | 328, 329 |
| A0120 | A0120-CS8_A_NH2_Alkyn | | 328 328, |
| | | | 328, 329 |
| A0121 | A0121-CS8_Z_N3_CBO′ | | 330, 328, |
| | | | 328, 331 |
| A0148 | A0148-CS9_U_NH2_imp | | 327, 332, |
| | | | 332, 333 |
| A0149 | A0149-CS10_U_NH2_imp | | 327, 334, |
| | | | 334, 335 |
| A0150 | A0150-CS11_U_NH2_imp | | 327, 336, |
| | | | 336, 337 |
| A0175 | A0175-CS13_U_NH2_imp | | 327, 338, |
| | | | 338, 339 |
| A0177 | A0177-CS15_U_NH2_imp | | 327, 340, |
| | | | 328, 329 |
| A0180 | A0180-CS18_U_NH2_imp | | 341 |
| A0032 | A0032-CBO1_palm | | 342 |
| A0119 | A0119-IO_test | | 343 |
| A0129 | A0129- | | 344 |
| A0184 | A0184-Hyb_Block_1 | | 345 |
| A0185 | A0185-Hyb_Block_2 | | 346 |
| A0186 | A0186-Hyb_Block_3 | | 347 |
| A0067 | A0067-prFw_IO_q_v2 | | 348 |
| A0068 | A0068-tpl_IO_q_v2 | | 349 |
| A0055 | A0055-prRv_IO_q | | 350 |
| A0071 | A0071-101_16N | | 351 |
| A0125 | A0125-SynIO_Fixed6N | | 352 |
| A0126 | A0126-SynIO_tpl_q | | 353 |
| A0127 | A0127-prFw_IO_q_v2 | | 354 |
| A0128 | A0128-SynIO_prRv_q | | 355 |

### Methods and Analysis Relating to Results Shown in Selected Figures

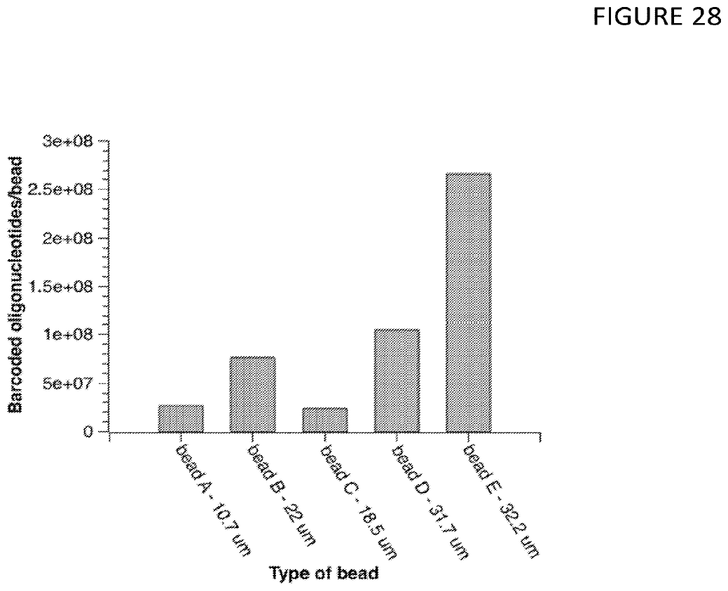

### FIG. 28

### Conjugation of A0147 (Multimeric Hybridization Molecule) to Beads

Various sized beads were conjugated with A0147 (multimeric hybridization molecule) according to what described in “Method 12-Peptide coupling of 14.5 μm carboxy magnetic beads with amino multimeric hybridization molecule”. Compared to that method, the amounts of reagents used were adjusted in a manner proportional to the surface area of the spherical beads calculated using the bead diameter.

### Annealing of A0119 to Conjugated Beads

An amount of beads containing 43 pmol of conjugated A0147 (estimated via the bead surface area) was placed in a tube, the beads were captured on a magnet and the supernatant was removed. The remaining beads were mixed with 66 μL of an annealing solution containing 390 pmol of A0119 (barcoded oligonucleotide) in an annealing buffer (150 mM NaCl, 15 mM Tris pH 7.5, 0.1 mM EDTA). The resulting mixtures were left on a rotator at 45° C. for 45 min and then at RT for another 30 min. The beads were collected using a magnet and the supernatant was transferred to a new tube placed on a magnet. This final supernatant was then used for the spectrophotometric measurements.

### Quantification of A0119 Annealed to the Beads

Absorbance at 260 nm was measured using 3 μL of solution on a microvolume spectrometer and the blank used was the annealing buffer. Absorbance at 260 nm was measured for all the supernatants obtained at the end of the annealing process and for a portion of the annealing solution (containing A0119) that was not used for hybridization with beads. By comparing the absorbance before and after the annealing step and by knowing the amounts of beads used it is possible to estimate the number of barcoded oligonucleotides annealed to each bead.

### Analysis

FIG. 28 is a graph showing barcoded oligonucleotides per bead with different types of beads having also different sizes. The graph shows that bead type and bead size have an effect on the number of barcoded oligonucleotides that a single bead-based multimeric barcoding reagent can carry.

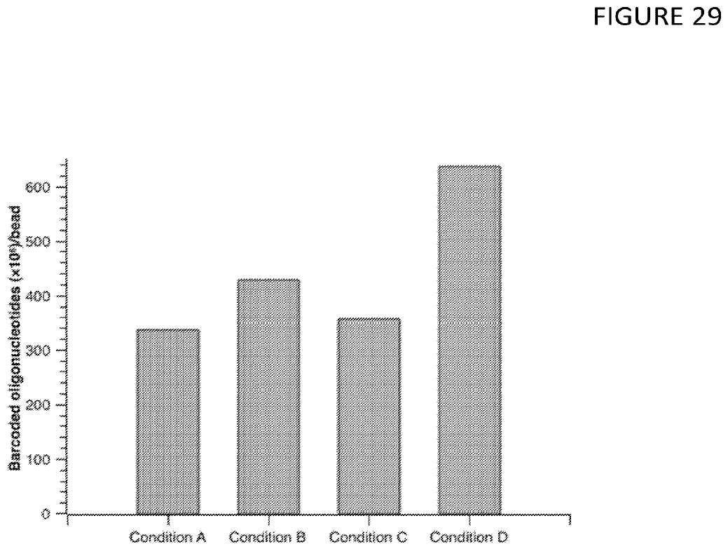

### FIG. 29

### Conjugation of A0147 (Multimeric Hybridisation Molecule) to Beads

32.2 μm beads were conjugated with A0147 (multimeric hybridisation molecule) according to what described in “Method 12-Peptide coupling of 14.5 μm carboxy magnetic beads with amino multimeric hybridisation molecule”. Compared to that method, the amounts of reagents used were adjusted in a manner proportional to the surface area of the spherical beads calculated using the bead diameter. Condition A used amounts directly proportional and equivalent to what described in Method 12. Compared to condition A, condition B used 0.38×A0147, 0.47×EDC and 0.46×volume. Compared to condition A, condition C used 0.75×A0147, 0.47×EDC and 0.46× volume. Compared to condition A, condition D used 0.63×A0147, 0.33×EDC and 0.33× volume.

### Hybridisation of A0119 and A0032 to Beads

859000 beads were annealed with 900 pmol of A0119 (barcoded oligonucleotide-6 μM final concentration) and with 300 pmol of A0032 (cell binding oligo-2 μM final concentration) in an annealing buffer (150 mM NaCl, 15 mM Tris pH 7.5, 0.1 mM EDTA). The resulting mixtures were left on a rotator at 60° C. for 10 min, then at 45° C. for 45 min and finally at RT for another 30 min. The beads were collected using a magnet and washed with 1×Dulbecco's PBS+3 mM MgCl2 three times. The beads were resuspended in 174 μL of the same PBS+Mg buffer.

### Quantification of A0119 Annealed to the Beads

The quantification was performed through a qPCR based method extending barcoded oligonucleotides like A0119 on a quantification template after their dehybridization from beads

### (Method 16-Multimeric Barcoding Reagent Barcoded Oligonucleotide Quantification Assay).

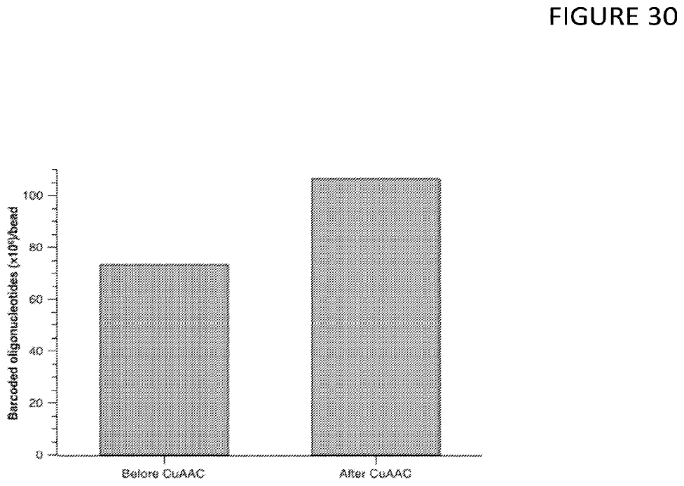

### FIG. 30

### Conjugation of a Multimeric Hybridisation Molecule to Beads and Subsequent Extension

The beads were prepared according to “Method 13-Attachment of multimeric hybridization molecules to 14.5 μm carboxy magnetic beads with subsequent extension with an orthogonal chemistry”.

### Hybridisation of A0119 and A0032 to Beads

For the “before CuAAC” sample:

300000 beads were annealed with 419 pmol of A0119 (barcoded oligonucleotide-4 μM final concentration) in an annealing buffer (150 mM NaCl, 15 mM Tris pH 7.5, 0.1 mM EDTA).

For the “after CuAAC” sample:

300000 beads were annealed with 733 pmol of A0119 (barcoded oligonucleotide-4 μM final concentration) and with 183 pmol of A0032 (cell binding oligo-1 μM final concentration) in an annealing buffer (150 mM NaCl, 15 mM Tris pH 7.5, 0.1 mM EDTA.

The resulting mixtures were left on a rotator at 60° C. for 10 min, then at 45° C. for 45 min and finally at RT for another 30 min. The beads were collected using a magnet and washed with 1× Dulbecco's PBS+3 mM MgCl2 three times. The beads were resuspended in 120 μL of the same PBS+Mg buffer.

### Quantification of A0119 Annealed to the Beads

The quantification was performed through a qPCR based method extending barcoded oligonucleotides like A0119 on a quantification template after their dehybridization from beads (Method 16-Multimeric Barcoding Reagent Barcoded Oligonucleotide Quantification Assay).

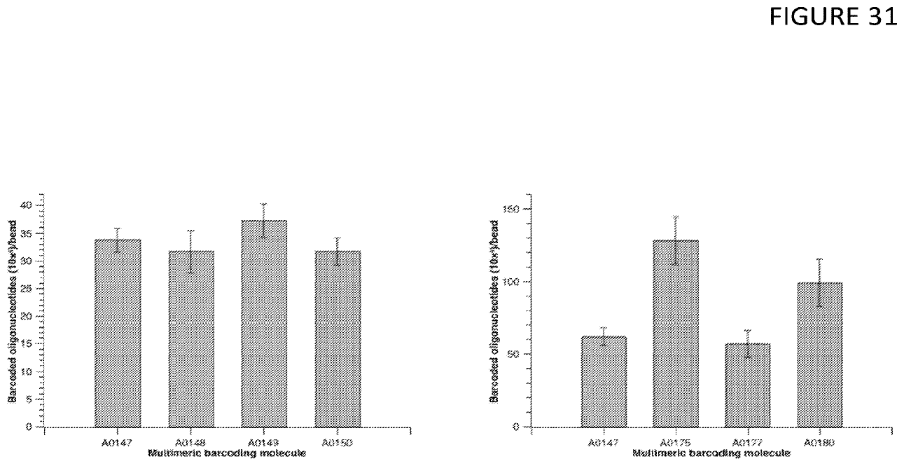

### FIG. 31

### Conjugation of Various Multimeric Hybridisation Molecule to Beads

Beads were conjugated with the various multimeric hybridisation molecules according to what described in “Method 12-Peptide coupling of 14.5 μm carboxy magnetic beads with amino multimeric hybridisation molecule”.

### Hybridisation of Barcoded Oligonucleotides and A0032 to Beads

For the figure on the left:

The beads were annealed with 0.0025 pmol/bead of A0129 (barcoded oligonucleotide-2.5 μM final concentration) and with 0.001 pmol/bead of A0032 (cell binding oligo-1 μM final concentration) in an annealing buffer (150 mM NaCl, 15 mM Tris pH 7.5, 0.1 mM EDTA). For the figure on the right:

The beads were annealed in 768 different annealing reactions, each with a different barcoded oligonucleotides of the same structure as A0129. The beads were annealed with 0.0008 pmol/bead of a barcoded oligonucleotide (0.25 μM final concentration) and with 0.0008 pmol/bead of A0032 (cell binding oligo-0.25 μM final concentration) in an annealing buffer (150 mM NaCl, 15 mM Tris pH 7.5, 0.1 mM EDTA).

The resulting mixtures were left on a rotator at 60° C. for 10 min, then at 45° C. for 45 min and finally at RT for another 30 min. The beads were collected using a magnet and washed with 1× Dulbecco's PBS+3 mM MgCl2 three times. The beads were resuspended in 1× Dulbecco's PBS +3 mM MgCl2. For the figure on the right, the 768 different beads were at the end pooled in a single tube.

### Quantification of Barcoded Oligonucleotides Annealed to the Beads

The quantification was performed through a qPCR based method extending barcoded oligonucleotides like A0129 on a quantification template after their dehybridization from beads (Method 16-Multimeric Barcoding Reagent Barcoded Oligonucleotide Quantification Assay).

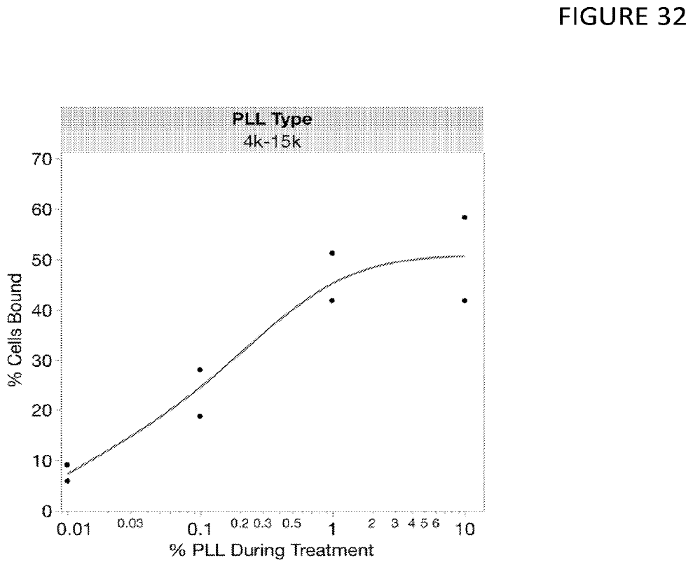

### FIG. 32

A library of 2.8 μm multimeric barcoding reagents was ‘PLL activated’ by incubating the library in a solution containing a specific concentration of 4 k-15 k Mw Poly-L-Lysine Hydrobromide. Each concentration of solution was prepared into a PBS+3 mM MgCl2 buffer. Prior to the incubation, the previous buffer (PBS+3 mM MgCl₂) was removed while the beads were pulled to a magnet. The beads were then resuspended into 2× the initial volume of the solution, placed onto a room temperature rotary mixer and incubated for 30 minutes. After the incubation, beads were pulled to magnet and the liquid was removed from the tube. The same volume of PBS+3 mM MgCl₂ was washed over the beads and then removed. Finally, the beads are resuspended in PBS+3 mM MgCl₂ to the initial volume used as input to the ‘activation’.

For the cell to multimeric barcoding reagent binding, 11 μL binding reactions were prepared to final concentrations of 1,000 cells/μL (in PBS) and 10,000 beads/ul (PBS+3 mM MgCl₂). The remaining volume of reactions that was not made from beads or cells was made up to 11 μLs with PBS. Samples were mixed 15 times with a P20 pipette with a volume of 10 μL. A 10 μL sample of the solution was used for haemocytometer assessment of reagent binding effectiveness and state

### Analysis

FIG. 32 is a graph showing that adjusting the concentration (v/v) of 4 k-15 k Poly-L-Lysine during a reagent ‘activation’ step impacts the cell-binding capability of treated reagents. Log scale shown. This demonstrates the high absolute levels of cell-binding between cells and reagents (as measured by the percentage and number of cells successfully bound to a multimeric barcoding reagent), and further demonstrates the effect that the presence (and concentration and/or amount) of the Poly-L-Lysine cell-binding moiety that is linked to said multimeric barcoding reagent(s) has on the relative rate/amount of such cell-binding by such multimeric barcoding reagents (i.e. the figure illustrates cell-binding mediated specifically by the presence/amount of a cell-binding moiety).

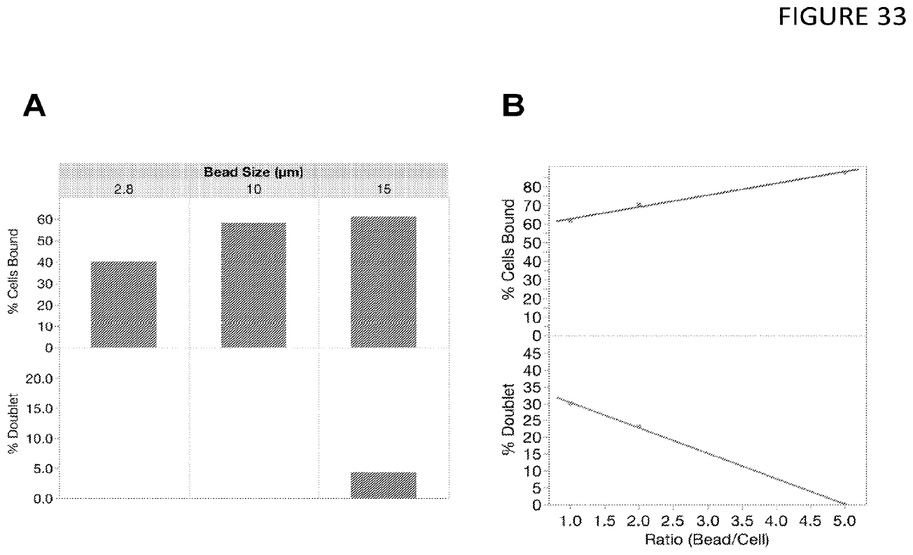

### FIG. 33A

Libraries of multimeric barcoding reagents containing a palmitate cell binding moiety were ‘PLL activated’ by incubating each library in a solution containing a 1% concentration of 4 k-15 k Mw Poly-L-Lysine Hydrobromide. Prior to the incubation, the previous buffer (PBS+3 mM MgCl₂) was removed while the beads were pulled to a magnet. The beads were then resuspended into 2× the initial volume of the solution, placed onto a room temperature rotary mixer and incubated for 30 minutes. After the incubation, beads were pulled to magnet and the liquid was removed from the tube. The same volume of PBS+3 mM MgCl₂ was washed over the beads and then removed. Finally, the beads are resuspended in PBS+3 mM MgCl₂ to the initial volume used as input to the ‘activation’.

Cells were mixed with multimeric barcoding reagents of various sizes (i.e. different physical diameters) and concentration to enable a final cell concentration of 500 cells/μL after mixing. To enable reagent-cell ratios of 20, 10 and 5, final bead concentrations of 10,000 beads/μL, 5,000 beads/μL and 2,500 beads/μL of the appropriate bead size were used respectively. The resulting solutions were prepared to 10 μLs with additional PBS+3 mM MgCl2. mixed 15 times with a P20 pipette set on 8 μLs. A 10 μL sample of the solution was used for haemocytometer assessment of reagent binding effectiveness and state.

### FIG. 33B

A library of 10.7 μm multimeric barcoding reagents was ‘PLL activated’ by incubating the library in a solution containing a 1% concentration of 4 k-15 k Mw Poly-L-Lysine Hydrobromide. Prior to the incubation, the previous buffer (PBS+3 mM MgCl2) was removed while the beads were pulled to a magnet. The beads were then resuspended into 2× the initial volume of the solution, placed onto a room temperature rotary mixer and incubated for 30 minutes. After the incubation, beads were pulled to magnet and the liquid was removed from the tube. The same volume of PBS+3 mM MgCl₂ was washed over the beads and then removed. Finally, the beads are resuspended in PBS+3 mM MgCl₂ to the initial volume used as input to the ‘activation’.

2.71 μLs of single cell solution at a concentration of 3687 cells/μL was added to a variable amount of PBS to settle to the bottom of a 0.2 mL tube at 4° C. over the course of 20 minutes. Following this incubation, the poly-Lysine activated library of 10.7 μm multimeric barcoding reagents (variable volumes to achieve desired bead/cell ratio and a final volume of 20 μL) were gently applied to the meniscus of the solution. This solution was then incubated for an additional 20 minutes at 4° C. to allow reagents to settle to the bottom of the tube. Following this incubation, the solution was disrupted by mixing 10 times with 10 μLs set on P20 pipette. A 10 μL sample of the solution was used for haemocytometer assessment of reagent binding effectiveness and state.

### Analysis

FIG. 33 shows graphs showing the results of cell binding experiments with variable binding conditions. A. High % cells bound with low % doublet was achieved with various bead sizes and ratios. For 2.8 μm beads, a bead/cell binding ratio of 20 was used. For 10 μm beads, a bead/cell binding ratio of 10 was used. For 15 μm beads, a bead/cell binding ratio of 5 was used. No doublets were observed in 2.8 μm bead or 10 μm bead conditions. B. With 10 μm beads in settling studies, a high % cells bound could be achieved, however at lower bead/cell ratios a high % doublet was observed.

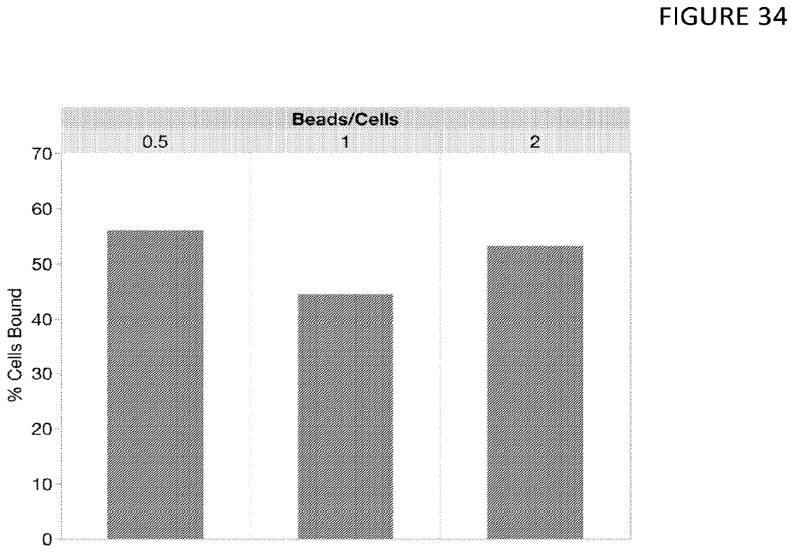

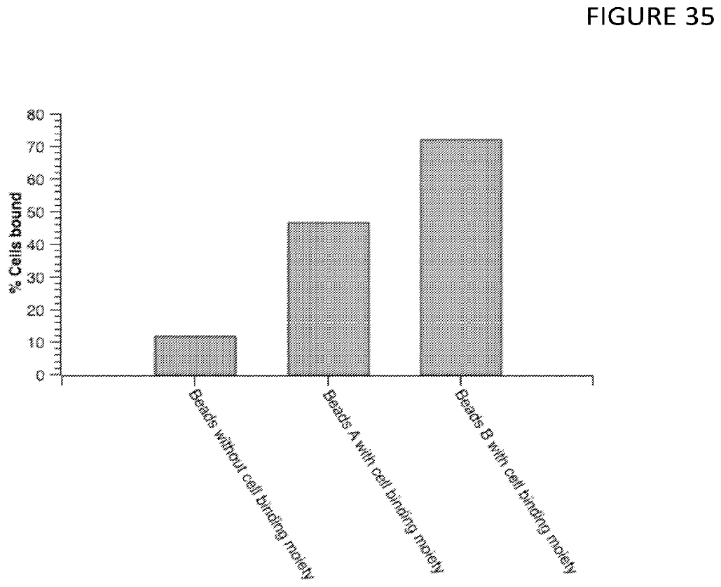

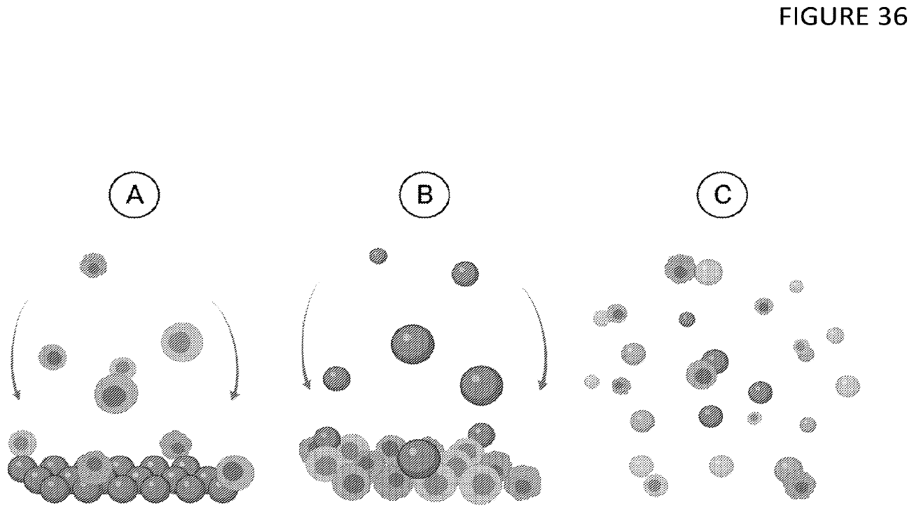

### FIG. 34

A library of 32.3 μm multimeric barcoding reagents (containing palmitate was prepared to three concentrations (1,000 beads/μL, 500 beads/μL and 250 beads/μL). Of which, 10 μL of each solution was moved to a 0.2 mL tube and the beads were allowed to settle to the bottom of the tube over the course of 20 minutes at 4° C. 10 μLs of a single NIH3T3 cell solution at a concentration of 500 cells/μL was then gently applied to the meniscus of the solution. This solution was then incubated for an additional 20 minutes at 4° C. to allow cells to settle to the bottom of the tube.

Following incubation, the solution was disrupted by mixing 5 times with 18 μL set on a P20 pipette. 10 μL samples were utilised for the haemocytometer-based assessment of reagent binding effectiveness and state.

### Analysis

FIG. 34 shows that in a 30 μm bead settling experiment a consistent % Cells Bound was achieved when the bead to cell ratio was reduced from 2 down to 0.5.

### FIG. 35

### Conjugation of A0147 (Multimeric Hybridization Molecule) to Beads

Beads were conjugated with A0147 (multimeric hybridization molecule) according to what described in “Method 12-Peptide coupling of 14.5 μm carboxy magnetic beads with amino multimeric hybridization molecule”.

### Hybridization of Barcoded Oligonucleotides to Beads

1000000 beads were annealed with 786 pmol of barcoded oligonucleotides (1 μM final concentration) in an annealing buffer. The annealing buffer used for beads A contained 150 mM NaCl, 15 mM Tris pH 7.5, 0.1 mM EDTA, 20% PEG 4000 while the annealing buffer used for beads B contained 150 mM NaCl, 15 mM Tris pH 7.5, 0.1 mM EDTA. The resulting mixtures were left on a rotator at 45° C. for 45 min and then at RT for another 30 min. The beads were collected using a magnet and washed with 1× Dulbecco's PBS+3 mM MgCl₂ three times. The beads were resuspended in 200 μL of the same PBS+Mg buffer.

### Addition of Poly-Lysine as Cell Binding Moiety to Beads

25 μL of hybridised beads were placed on a magnet and the supernatant was removed. The beads were then mixed with 50 μL of a 1% 150000 poly-lysine solution (in 1× Dulbecco's PBS+3 mM MgCl₂). This mixture was left on a rotator for 30 min (RT). After the incubation, the beads were pulled to magnet and the supernatant was removed from the tube. 50 μL of the same PBS+Mg buffer were used to wash the beads, which were then resuspended in 25 μL of the PBS+Mg buffer.

### Cell-Bead Binding

Cells were prepared to a concentration of 5000 cells/μL, while the beads were diluted to a concentration of 1000 beads/μL. 5 μL of cells were mixed with 5 μL of beads, the mixing was performed by mixing a volume of 8 μL 10 times with a 20 μL pipette. The entirety of that mixture was then used for haemocytometer assessment of reagent binding effectiveness and state. Unconjugated beads were used for the control without poly-lysine (cell binding moiety).

### Analysis

FIG. 35 shows the effect of adding a cell binding moiety to the % of cells bound by a bead based multimeric barcoding reagent. The presence of the same poly-lysine based cell binding moiety increases the binding compared to “naked” beads. Beads B used an improved cell bead moiety addition method compared to beads A.

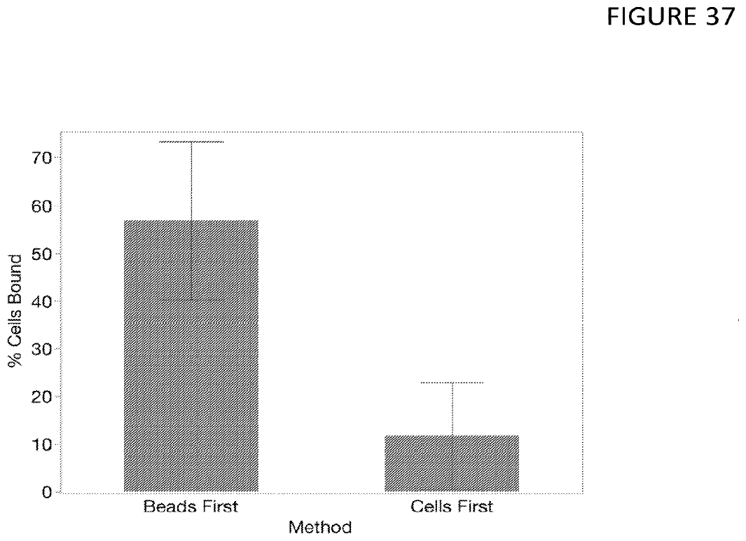

### FIG. 37

Aliquoted 14 μLs PBS to 0.2 mL PCR tube. Either 4 μLs of beads in PBS+3 mM MgCl2 (library of multimeric barcoding reagents) (5000 b/μL) or 2 μLs of cells in PBS (5000 cells/μL) were aliquoted into the tube. The cells or beads were allowed to settle to the bottom of the tube for 20 minutes at 4° C. The alternative species (not already aliquoted) was then gently applied to the meniscus of the solution. This solution was then incubated for an additional 20 minutes at 4° C. Following incubation, the solution was disrupted by mixing 6 times with 10 μL set on a P20 pipette. 10 μL samples were utilised for the haemocytometer-based assessment of reagent binding effectiveness and state.

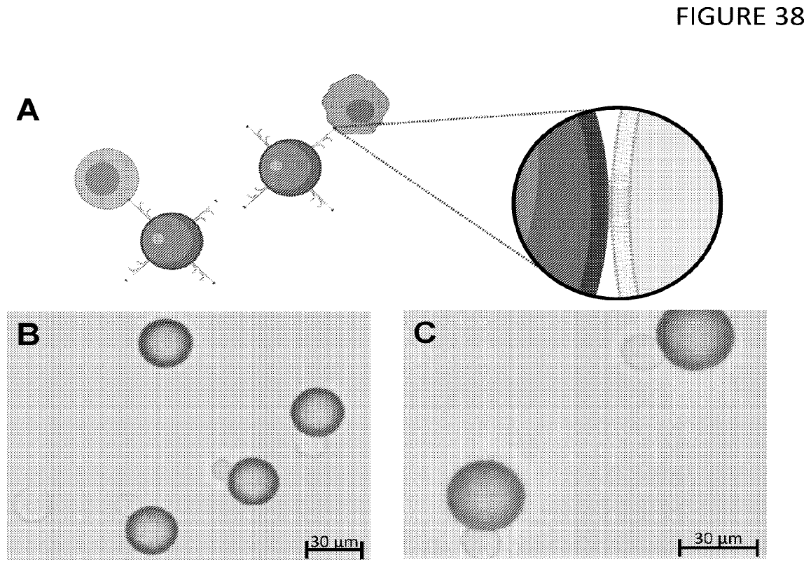

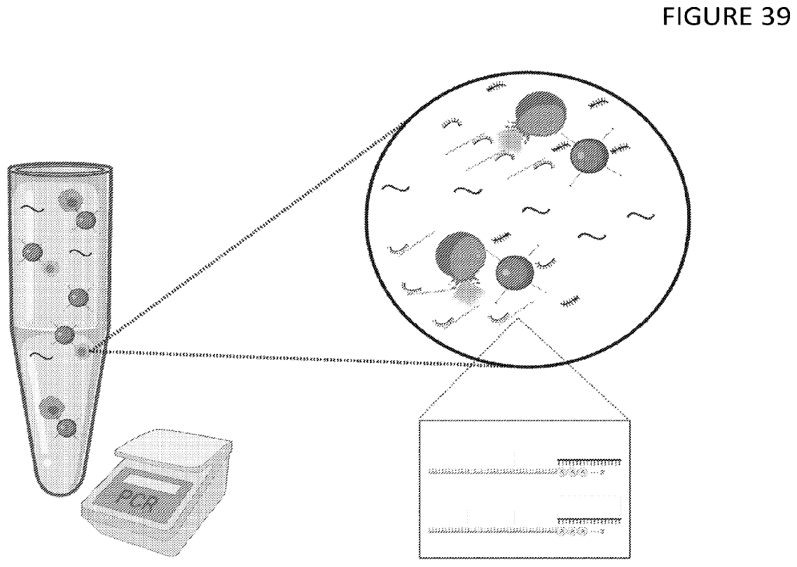

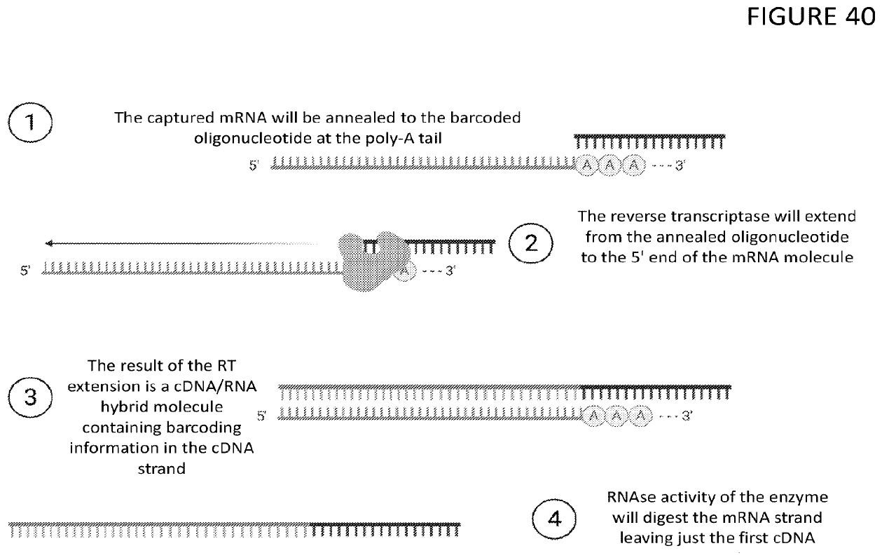

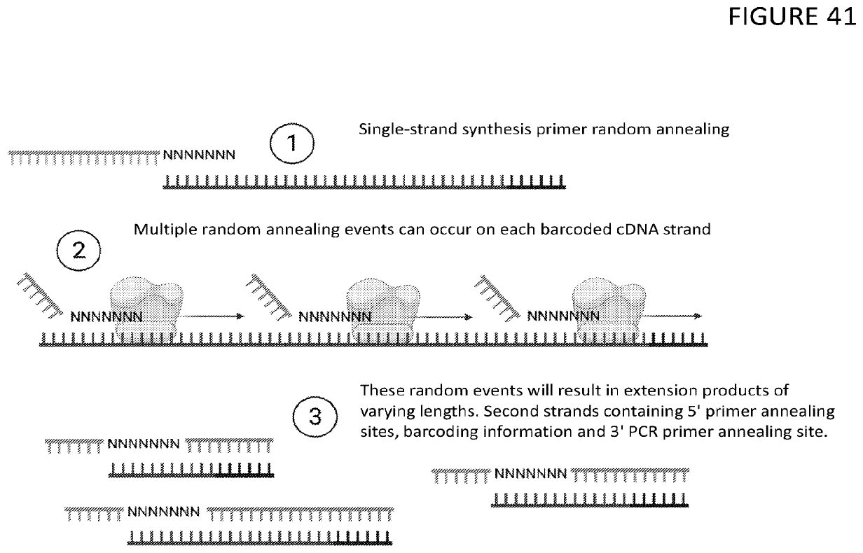

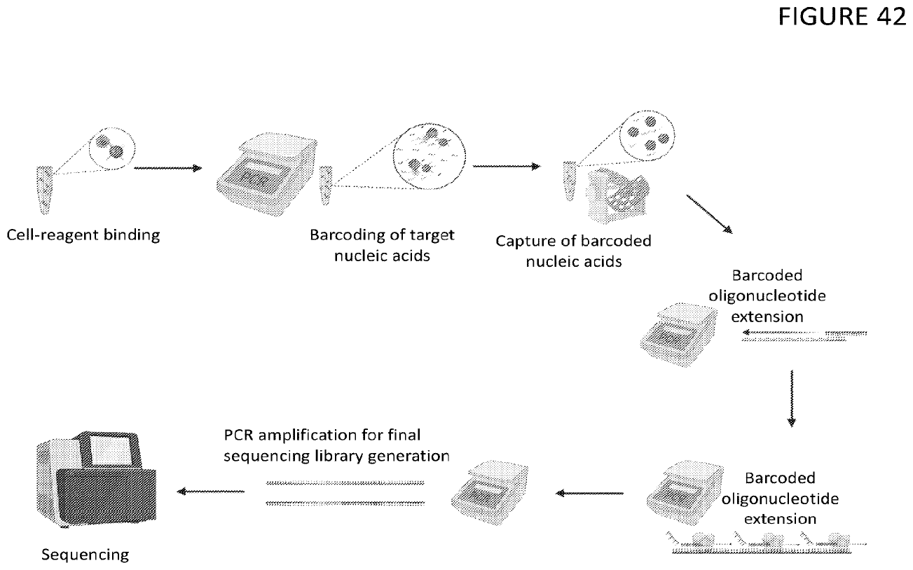

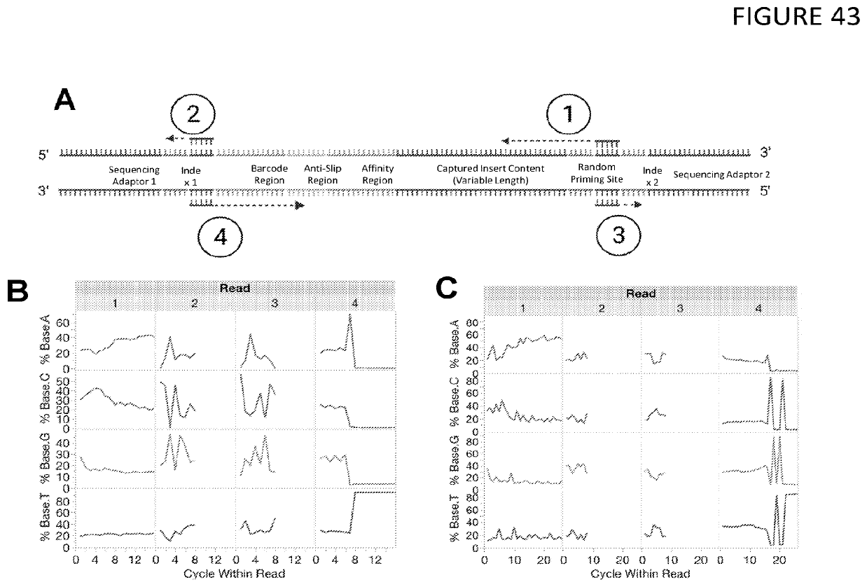

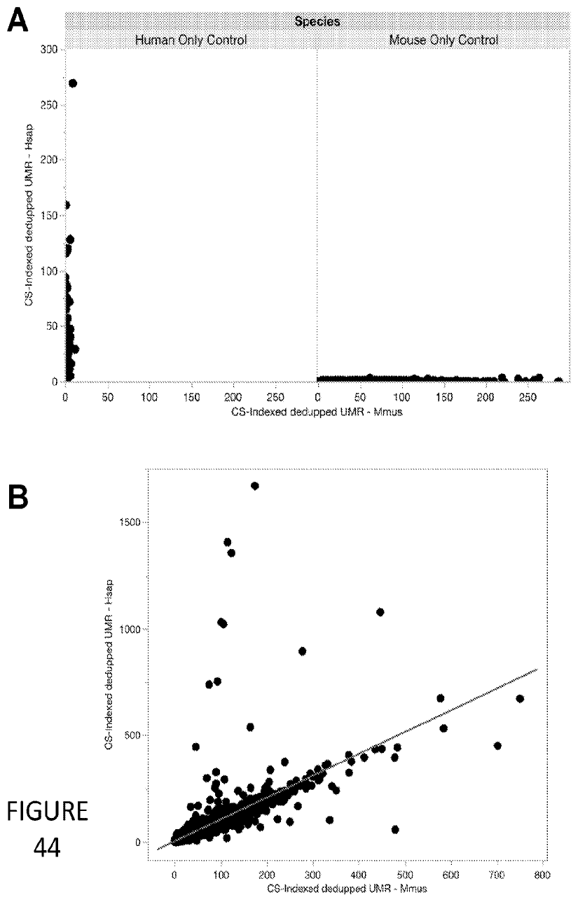

### FIG. 38

A 50% HEK293 and 50% NIH3T3 single-cell solution was prepared. In a 0.2 mL tube, 10 μLs of 30 μm beads (library of multimeric barcoding reagents) at a concentration of 250 beads/μL were allowed to settle to the bottom of the tube for 20 minutes at 4° C. Following this incubation, 10 μLs of the species mixed cell solution (500 cells/μL) was then gently applied to the meniscus of the solution. This solution was then incubated for an additional 20 minutes at 4° C. to allow cells to settle to the bottom of the tube. Following incubation, the solution was disrupted by mixing 5 times with 18 μL set on a P20 pipette. 10 μL samples were utilised for the haemocytometer-based assessment of reagent binding effectiveness and state. Images were taken at 40× magnification.

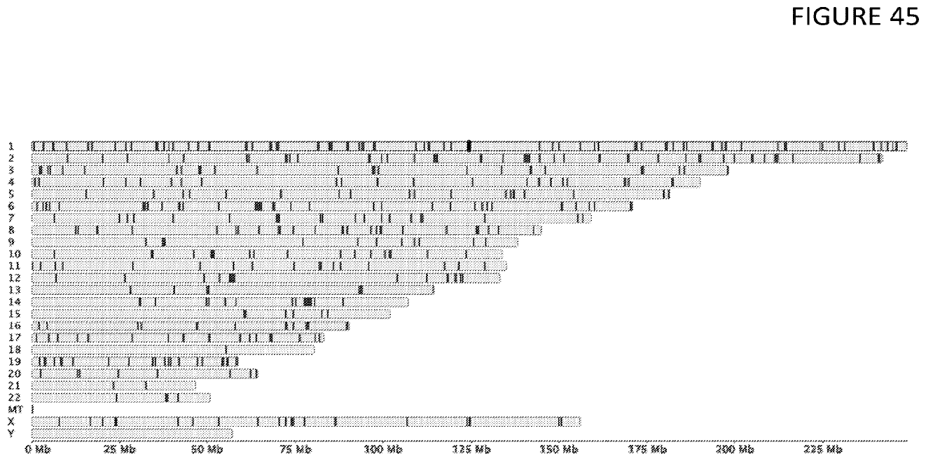

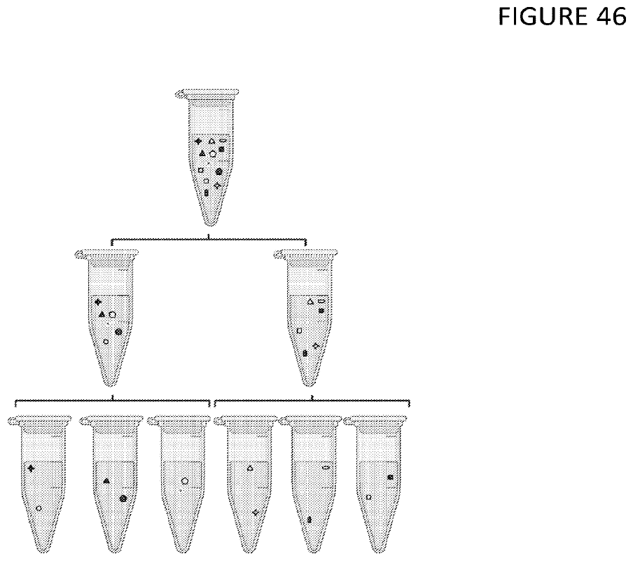

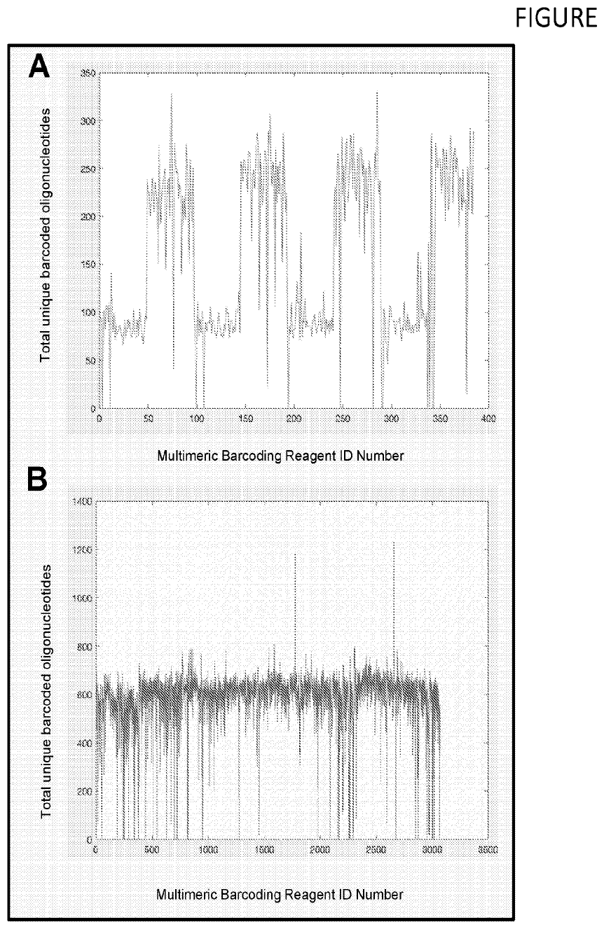

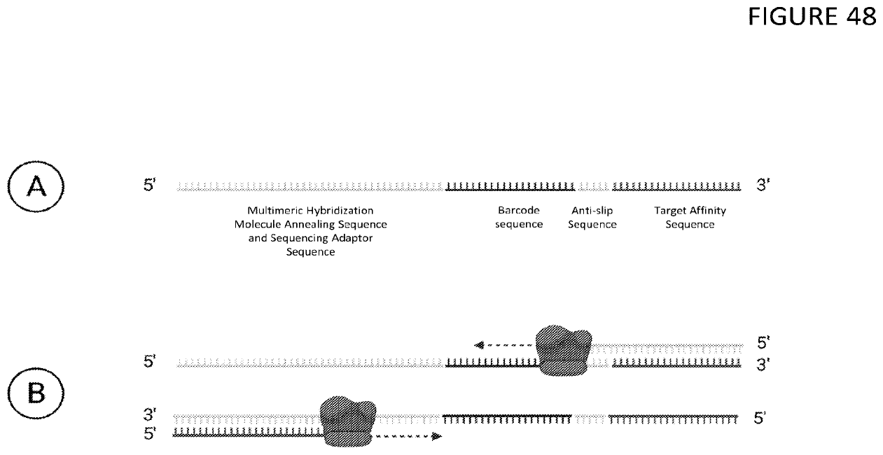

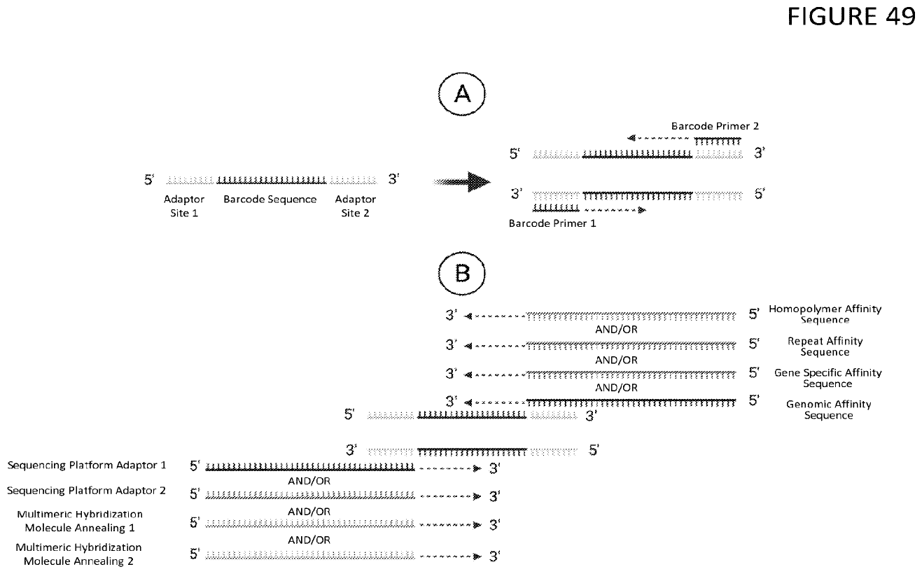

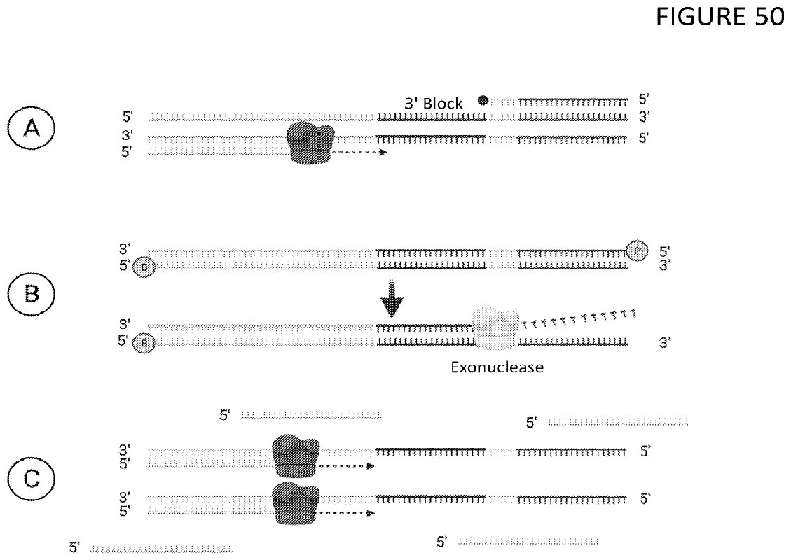

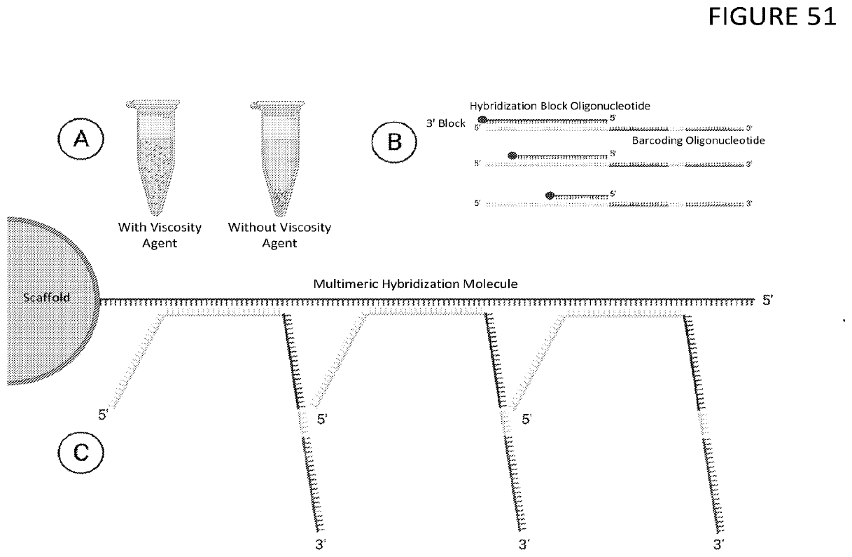

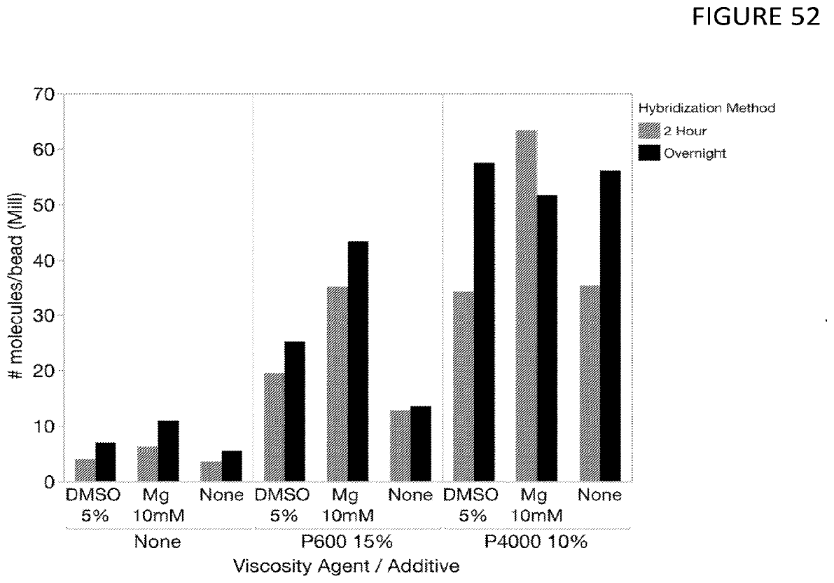

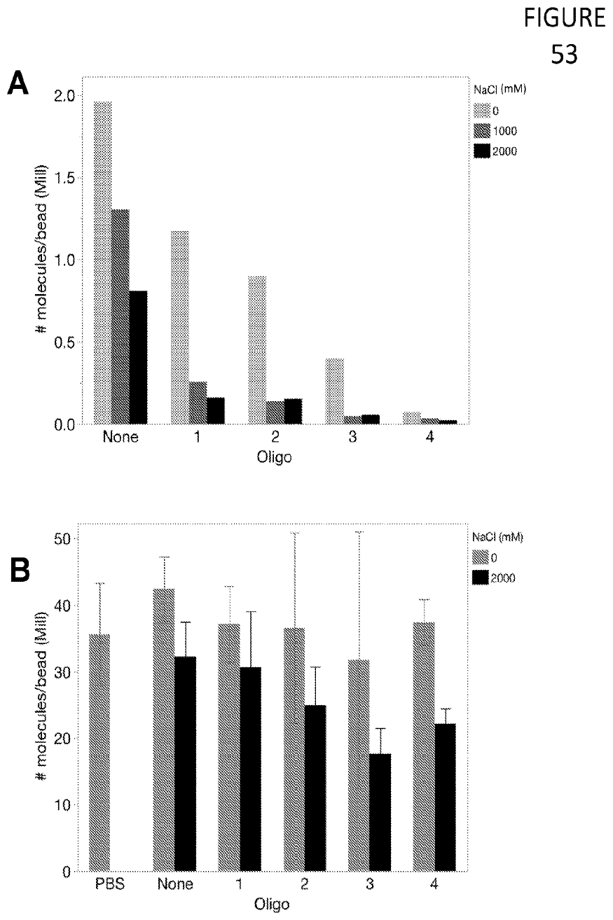

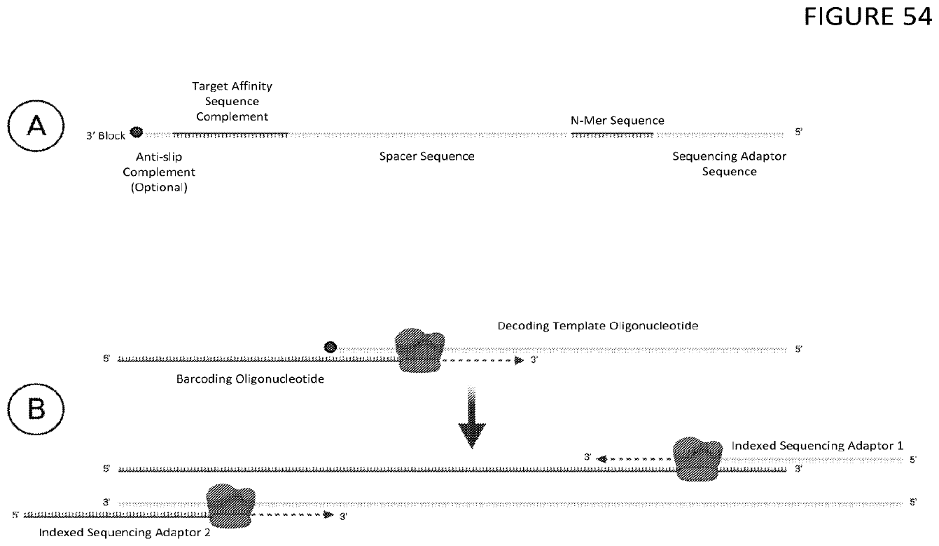

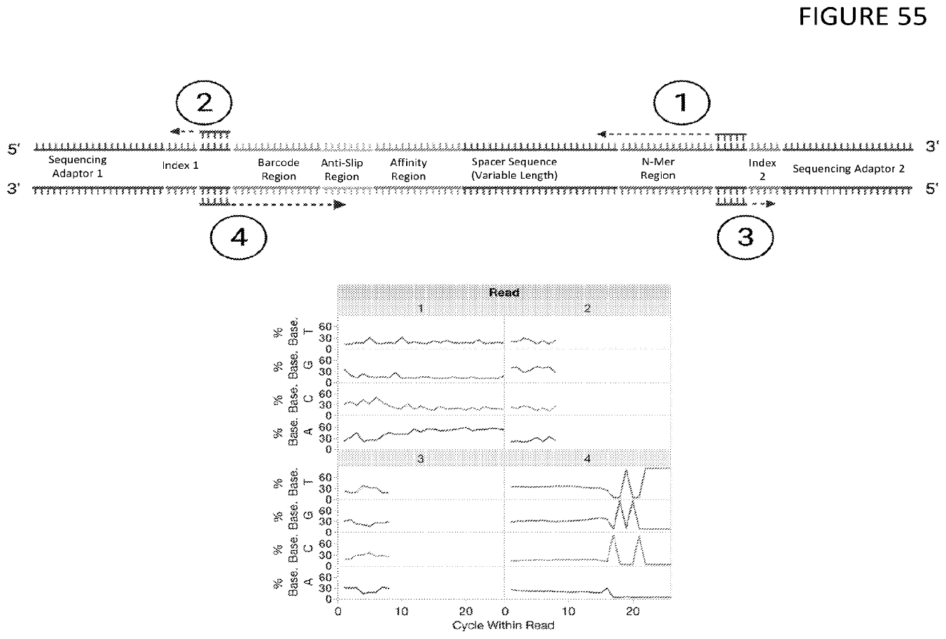

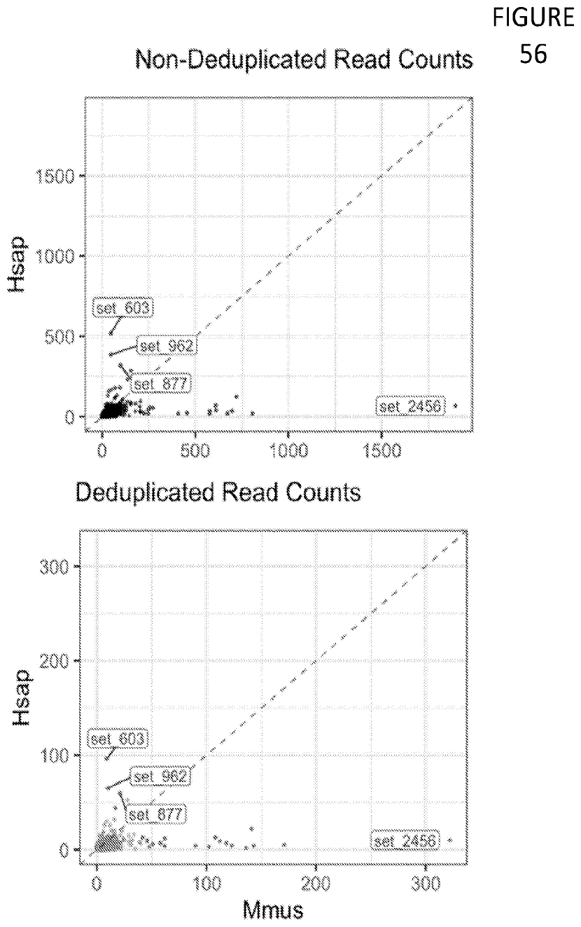

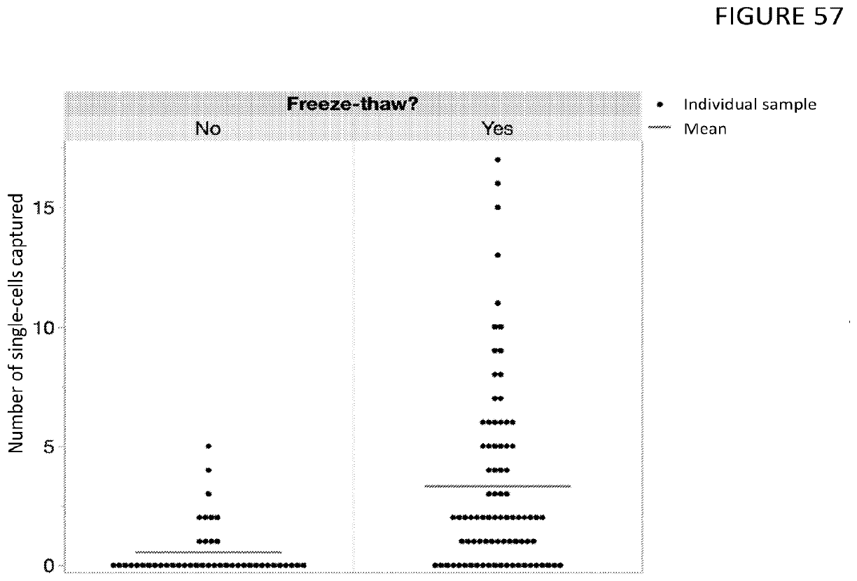

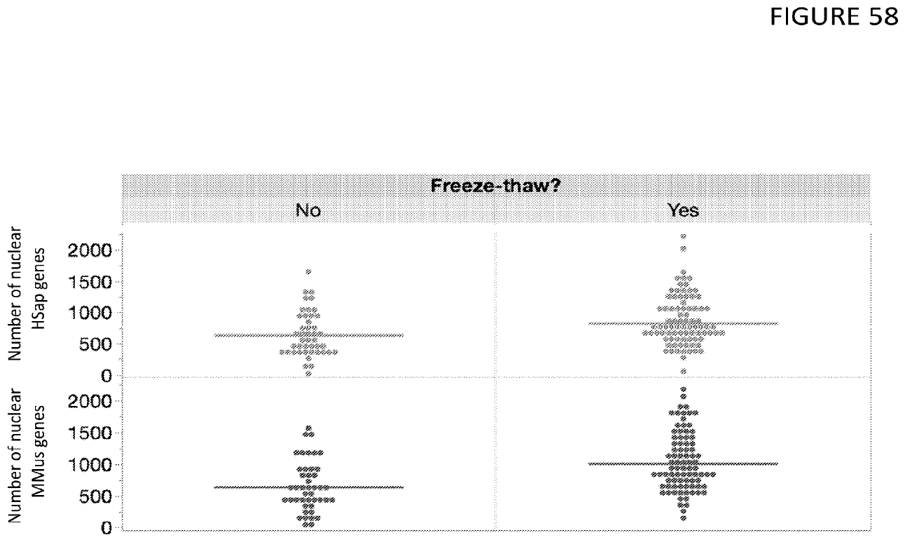

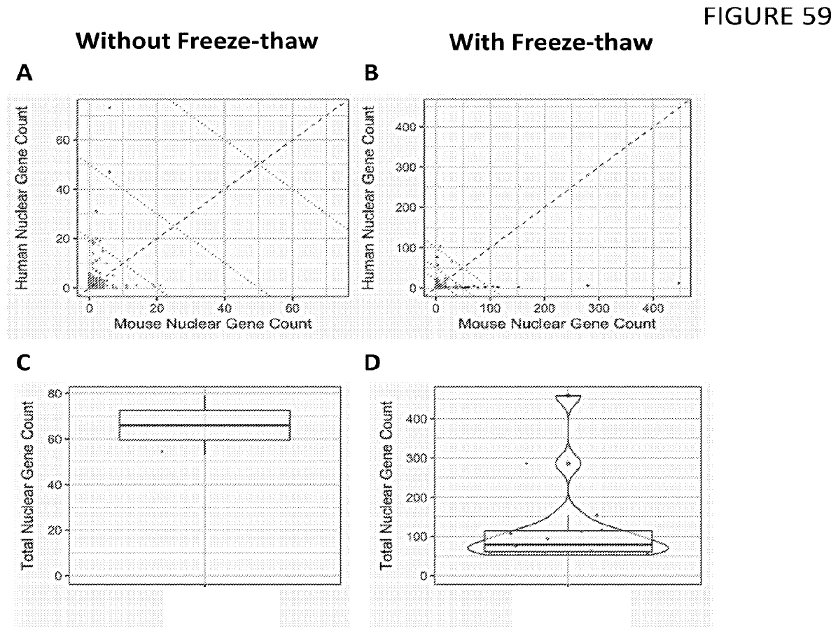

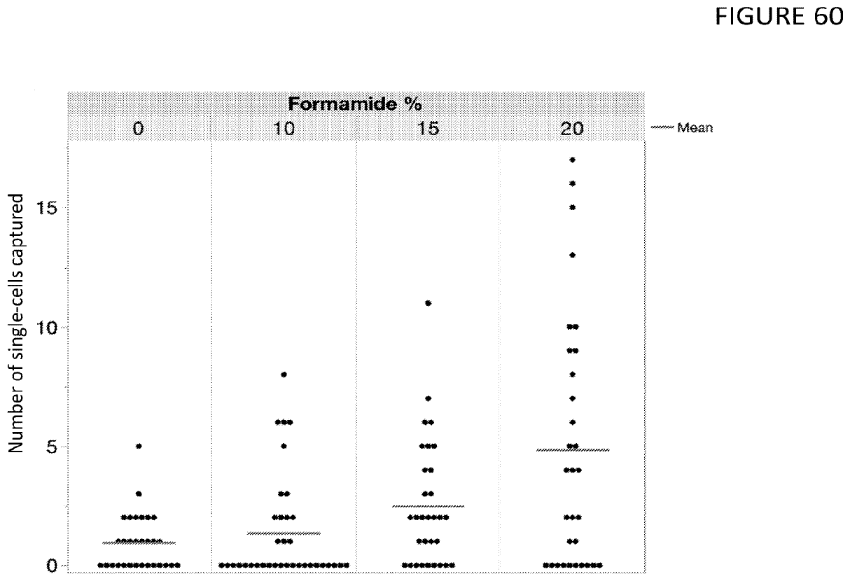

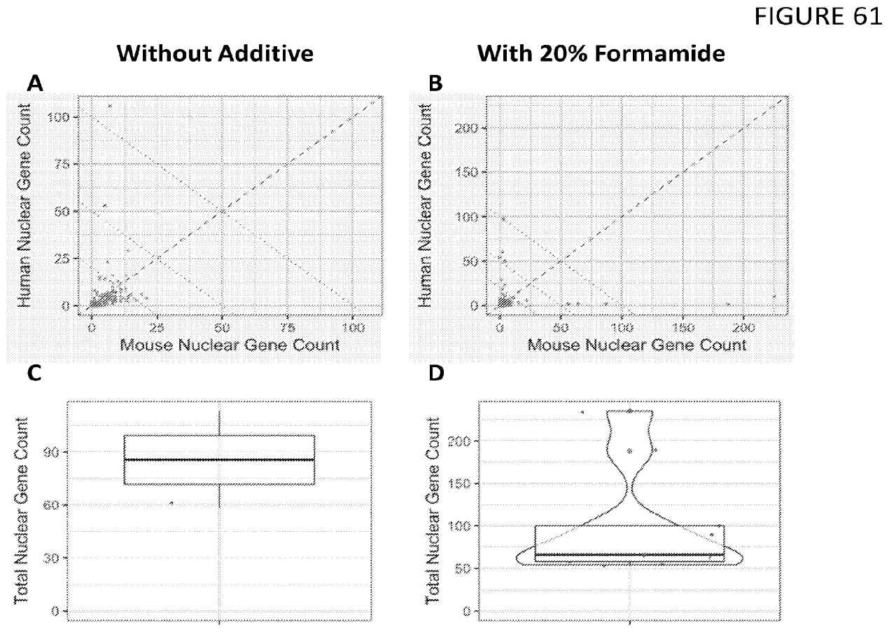

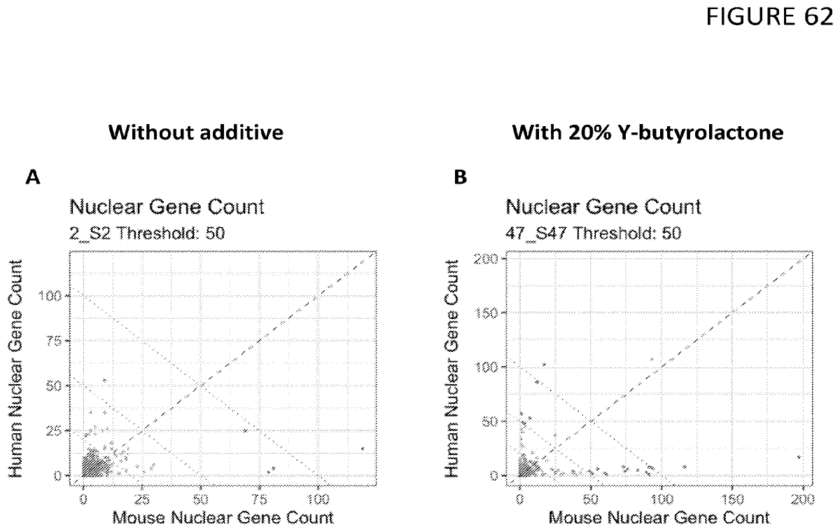

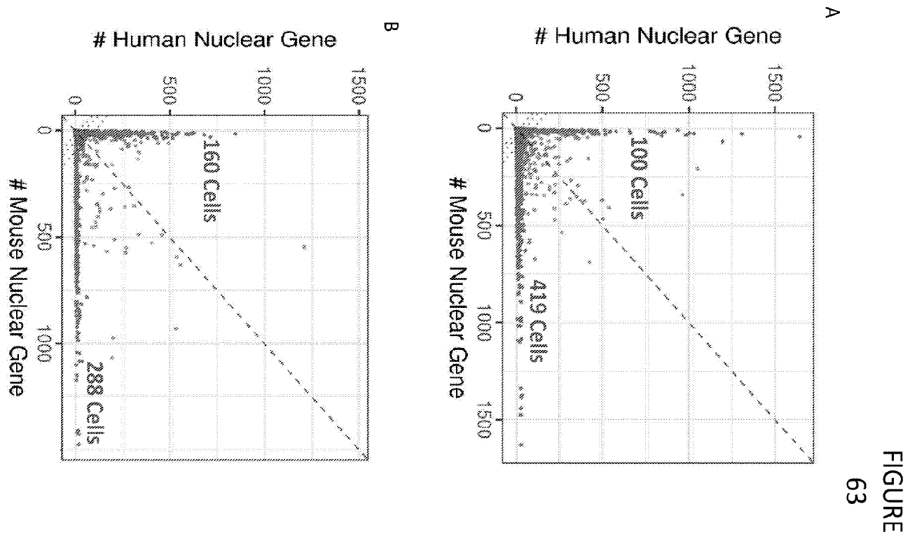

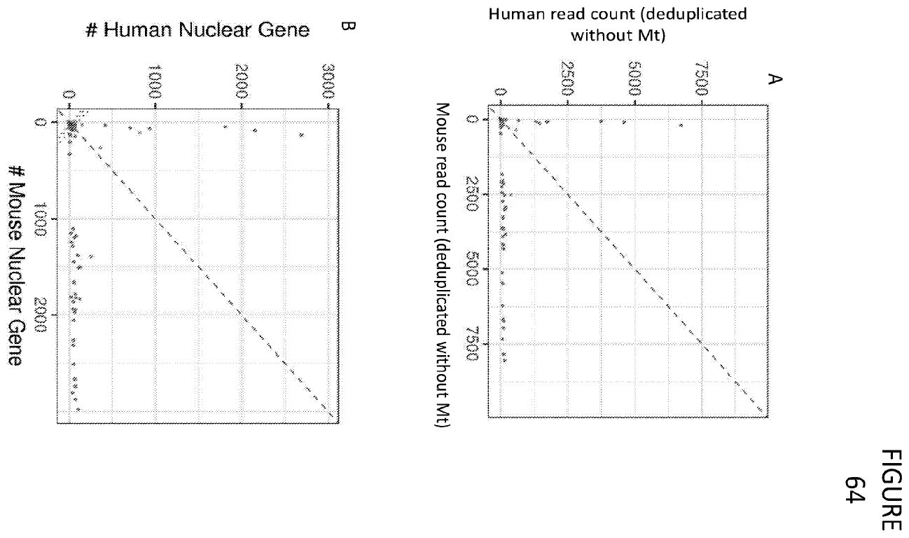

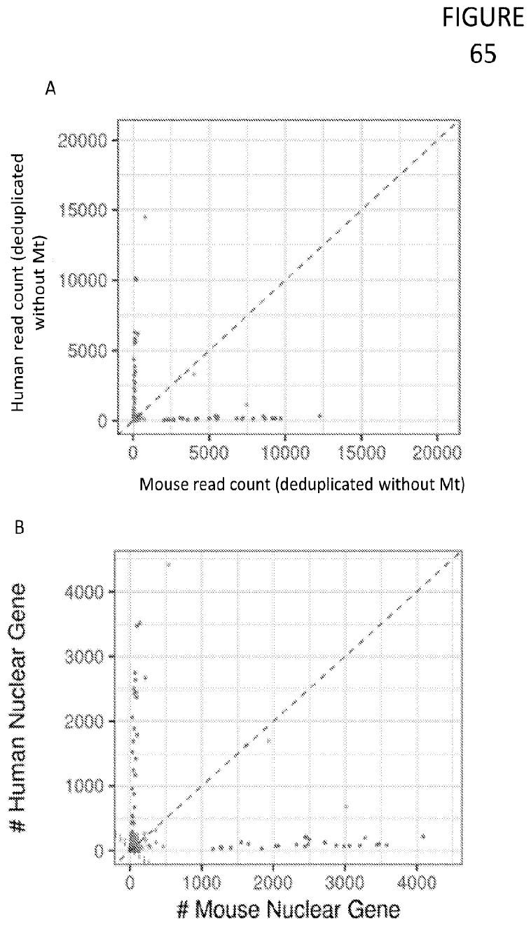

### FIGS. 44 and 45

### FIG. 44A

10 microliters of a single-cell suspension of cells of interest (either human cells alone or mouse cells alone) in PBS (at a concentration of 500 cells/ul) were carefully distributed across the top surface of a 10 microlitre reagent layer consisting of multimeric barcoding reagents (Spherotech 14.5 μm carboxy magnetic beads peptide coupled with amino multimeric hybridization molecules, consisting of a library of 768 barcoded oligonucleotide sets and a palmitate cell-binding moiety) at a concentration of 1,000 reagents/ul, for 20 minutes at 4° C. After 20 minutes, 30 microlitres of PBS was added and the whole 50 microlitres was mixed by pipetting up and down 10 times. A 2.5 microlitre aliquot of the cell-reagent mixture (comprising approximately 250 cells and 500 multimeric barcoding reagents) was transferred to a new PCR tube and 100 microlitres of cold lysis and indexing solution (comprising 100 millimolar Tris-HCl PH 7.5, 10 millimolar EDTA, 150 millimolar LiCl, 0.001% BRIJ58, 30% PEG 4000) was added and mixed by gently pipetting up and down 10 times.

The PCR tube containing the cells and multimeric barcoding reagents diluted into lysis and indexing solution was placed on a thermal cycler and incubated at 65° C. for 30 seconds, 45° C. for 1 minute, 35° C. for 3 minutes and then held at 20° C.

Following capture of the indexed mRNA using 4 microlitres of mRNA capture reagents, reverse transcription, second strand synthesis, PCR amplification and PCR for attachment of sequencing adapters was carried out as per Method 14 (“Assay for single cell whole transcriptome sequencing”) to generate libraries for sequencing.

Sequencing data was processed using a custom pipeline whereby reads were demultiplexed based on the barcode sequence to generate reads for single cells, and then mapped to a combined mouse-human custom genome. Mapped reads were then deduplicated and the uniquely mapped reads (UMR) for each species was plotted against the other.

### FIG. 44B & FIG. 45

10 microliters of a single-cell suspension of a 1:1 mixture of human and mouse cells in PBS (at a concentration of 500 cells/ul) were carefully distributed across the top surface of a 10 microlitre reagent layer consisting of multimeric barcoding reagents (Spherotech 14.5 μm carboxy magnetic beads peptide coupled with amino multimeric hybridization molecules, consisting of a library of 3000 barcoded oligonucleotide sets and a palmitate cell-binding moiety) at a concentration of 1,000 reagents/ul) for 20 minutes at 4° C. After 20 minutes, the whole 20 microlitres was mixed by pipetting up and down 10 times. A 4 microlitre aliquot of the cell-reagent mixture (comprising approximately 1000 cells and 2000 multimeric barcoding reagents) was transferred to a new PCR tube and 100 microlitres of cold lysis and indexing solution (comprising 100 millimolar Tris-HCl PH 7.5, 10 millimolar EDTA, 150 millimolar LiCl, 0.001% BRIJ58, 30% PEG 4000) was added and mixed by gently pipetting up and down 10 times. The PCR tube containing the cells and multimeric barcoding reagents diluted into lysis and indexing solution was placed on a thermal cycler and incubated at 65° C. for 30 seconds, 45° C. for 1 minute, 35° C. for 3 minutes and then held at 20° C.

Following capture of the indexed mRNA using 4 microlitres of mRNA capture reagents, reverse transcription, second strand synthesis, PCR amplification and PCR for attachment of sequencing adapters was carried out as per Method 14 (“Assay for single cell whole transcriptome sequencing”) to generate libraries for sequencing.

Sequencing data was processed using a custom pipeline whereby reads were demultiplexed based on the barcode sequence to generate reads for single cells, and then mapped to a combined mouse-human custom genome. Mapped reads were then deduplicated and the uniquely mapped reads (UMR) for each species was plotted against the other.

In FIG. 45, a single cell that was classified as a human cell from the 1:1 mouse and human library (plot in FIG. 44B) was visualised on a genome browser to demonstrate the coverage of reads across the genome.

### Analysis

FIG. 44 shows: A. Single species de-duplicated uniquely mapped reads for human (Hsap; Homo Sapiens) or mouse (Mmus; Mus Musculus) only control samples; and B. Human/Mouse species mixing results. In the background context of multiple species target strands, several multimeric barcoding reagents have a heavy species bias, indicating successful single cell sequencing with multimeric barcoding reagents.

The data shown in FIG. 44 demonstrates that multiple individual multimeric barcoding reagents have labelled a large number of target nucleic acids from respective bound single cells, with such single-cell-bound multimeric barcoding reagents identified by their placement along or close to the vertical or horizontal axes. These represent single mouse or human cells with a high number of uniquely mapped reads (UMR) that have been derived from messenger RNA barcoding by a specific multimeric barcoding reagent, thereby demonstrating high-fidelity and sensitive barcoding of messenger RNA molecules from single cells within a solution comprising a large number of cells and a large number of multimeric barcoding reagents.

FIG. 45 shows human cell genomic coverage of a successful single cell mRNA capture. Reads are identified across the coverage of chromosomes.

The data shown in FIG. 45 demonstrates widespread and unbiased coverage across the transcriptome that can be achieved for a single cell that has had its constituent messenger RNA barcoded by the barcoded oligonucleotides comprised within a multimeric barcoding reagent in a barcoding process.

### FIG. 47

In the figures shown both images show the number of unique barcoded oligonucleotides on each multimeric barcoding reagent across all the multimeric barcoding reagents amplified in the decoding reaction. During analysis of decoding sequencing data, a unique barcoded oligonucleotide is called only when there are at least 2 reads per barcoded oligonucleotide.

In image A the number of unique barcoded oligonucleotides switches between approximately 100 and 250 per 50 multimeric barcoding reagents. This demonstrates the ability to fine-tune the diversity generation step of the multimeric barcoding reagent manufacturing process to give varying diversity of barcoded oligonucleotides available on each multimeric barcoding reagent. To generate this data two master mixes containing 5×HF buffer (NEB), dNTPs, A0065, A0066 and Phusion polymerase (NEB) and water were combined. For each of the two master mixes two different volumes of a dilution of A0064 at 500 molecules/ul were added to give a total of either 100 molecules per diversity generation reaction or 250 molecules per diversity generation reaction. These master mixes were distributed across a 384-well PCR plate and heated to 98° C. for 30 seconds, then 33 cycles of 98° C. for 10 seconds, 62° C. for 10 seconds and 72° C. for 15 seconds. There is a final elongation step of 72° C. for 10 seconds. A quarter of each reaction was carried through to a second PCR reaction for linear amplification of the barcoded oligonucleotide. This reaction, along with the aliquot from the diversity generation reaction, contains 5×HF buffer (NEB), dNTPs, A0066, Phusion polymerase (NEB) and water. The sample is subject to 98° C. for 30 seconds, then 20 cycles of 98° C. for 10 seconds, 62° C. for 10 seconds and 72° C. for 15 seconds. There is a final elongation step of 72° C. for 10 seconds. Finally, an aliquot from each well is combined with a master mix containing Taq reaction buffer (NEB), dNTPs, Hot Start Taq polymerase, A0105, nuclease free water and a pair of unique index Illumina® sequencing adapters. The reaction is heated in a thermocycler for 95° C. for 1 minute, 50° C. for 30 seconds, 68° C. for 20 seconds then 13 cycles of 95° C. for 15 seconds and 68° C. for 20 seconds. There is a final elongation step of 68° C. for 30 seconds. All 384 reactions are pooled and purified using SPRI beads with 80% ethanol washes and eluted in water. This library is then able to be quantified and diluted appropriately for the platform intended for sequencing of the pool. This is the sequencing data which resulted in the clear division between input molecules as seen in figure A when calling unique barcoded oligonucleotide sequences.

In image B the same method as described for image A above was used except there was a single input amount of 600 molecules of A0064 for each reaction and this was also done at a scale of 3,072 reactions. The resulting analysed decoding sequencing data showed approximately 600 seed molecules were generated at the diversity generation step for each reaction with the exception of several drop-out reactions, maintaining an even population of unique barcoded oligonucleotides per multimeric barcoding reagent in the library.

### FIG. 52

FIG. 52 shows data from the optimisation of the annealing of barcoded oligonucleotides to magnetic beads. This data was generated by setting up annealing reactions with an aliquot of a linear amplification reaction (as described in Method 15b-Linear Amplification of the diversity generation reaction) combined with a common master mix for each optimisation condition containing multimeric hybridization molecule conjugated 14.5 μm beads, hybridization buffer (containing 150 mM Tris-HCl pH 7.5, 1.5M sodium chloride and 1 mM EDTA) and A0032. For each of these master mixes a varying amount of water, Magnesium Chloride (Mg), Dimethyl sulfoxide (DMSO), polyethylene glycol (PEG) 4000 and polyethylene glycol (PEG) 600 was added to the master mixes to give the combination of conditions shown in the figure. Each of these reaction formulations was incubated with rotation for either 10 minutes at 60° C. then 45 minutes at 45° C. then left to cool to room temperature for 30 minutes, or alternatively incubated with rotation for either 10 minutes at 60° C. then 45 minutes at 45° C. then left to cool to room temperature and left rotating overnight. Each reaction was then cleaned up to remove unannealed barcoded oligonucleotides in solution so only the annealed barcoded oligonucleotides remained. The number of barcoded oligonucleotides on each bead for each optimisation condition was quantified by qPCR and compared to give the results presented in FIG. 52.

### Analysis

In FIG. 52 different molecular additives (such as Magnesium salt and DMSO) and/or viscosity agents were added to a reaction comprising the annealing of barcoded oligonucleotides to multimeric hybridization molecules to observe the resulting effect this had on the amount of barcoded oligonucleotides so annealed. Each annealing reaction condition was also tested with two different time profiles. The results show in the context of bead-based reagent scaffolds, polyethylene glycol 4000 at 10% v/v yielded the highest numbers of barcoded oligonucleotides per multimeric hybridization molecule following the annealing reaction using the two-hour time profile. This demonstrates optimisation of the annealing reaction to make annealing of barcoded oligonucleotides a more efficient manufacturing process. A higher number of barcoded oligonucleotides on each multimeric hybridization reagent allows for more barcoded oligonucleotides to be available for indexing of nearby nucleic acids, this will contribute positively to the sensitivity of the technology.

### FIG. 53

### A

Quenching solutions (containing 1 μM concentrations of appropriate hybridisation blocking oligonucleotide) were prepared into the appropriate solution (variable concentrations of MgCl2 and NaCl across the formulations).

A master mix containing multimeric hybridisation molecules conjugated to 14.5 μm beads (636.5 b/μL) was prepared (20% PEG 4 k, 1M NaCl, 30 mM Tris pH 7.5, 0.2 mM EDTA, 20 mM MgCl2). 20 μLs of this master mix was aliquoted to each well. 48 μLs of selected quenching solution was added to selected well onto the master mix solution and mixed. 20 μLs pools of amplified barcoded oligonucleotides (˜500 nM concentration) were then mixed into the solution and the solution was left for 5 minutes at room temperature. Following incubation, the solutions were placed onto a magnet, beads were pulled to the side and the solution was removed without disturbing the beads. 88 μLs of PBS+3 mM Mg was added to the solution and then removed twice to wash any residual buffer. The beads were removed from the magnet and resuspended into 40 μLs of PBS+3 mM Mg. Bead concentrations were then quantified before using the beads as an input into Method 16—‘Multimeric Barcoding Reagent Barcoded Oligonucleotide Quantification Assay’.

### B

Quenching solutions (containing 1 μM concentrations of appropriate hybridisation blocking oligonucleotide) were prepared into the appropriate solution (variable concentrations of NaCl across the formulations).

A master mix containing multimeric hybridisation molecules conjugated to 14.5 μm

Beads (636.5 b/μL) was prepared (20% PEG 4 k, 1M NaCl, 30 mM Tris pH 7.5, 0.2 mM EDTA, 20 mM MgCl2). 20 μLs of this master mix was aliquoted to each well. 20 μLs of amplified barcoded oligonucleotides (˜500 nM concentration) were then added to the solution and mixed. The plate was sealed and then exposed to a thermal profile which cools the sample from 70° C.-20° C. over the course of 1 hour on a thermal cycler instrument. The plate was removed from the incubation and then 40 μLs of the selected quenching solution was applied to the solutions and mixed. The solution was left to incubate for 5 minutes at room temperature.

Following incubation, the solutions were placed onto a magnet, beads were pulled to the side and the solution was removed without disturbing the beads. 80 μLs of PBS+3 mM Mg was added to the solution and then removed twice to wash any residual buffer. The beads were removed from the magnet and resuspended into 40 μLs of PBS+3 mM Mg. Bead concentrations were then quantified before using the beads as an input into Method 16-‘Multimeric Barcoding Reagent Barcoded Oligonucleotide Quantification Assay’.

### FIG. 56

10 microliters of a single-cell suspension of a 1:2 mixture of human and mouse cells in PBS (at a concentration of 500 cells/μl) were carefully distributed across the top surface of a 10 microlitre reagent layer consisting of multimeric barcoding reagents (Spherotech 14.5 μm carboxy magnetic beads peptide coupled with amino multimeric hybridization molecules, consisting of a library of 3000 barcoded oligonucleotide sets and a palmitate cell-binding moiety) at a concentration of 1,000 reagents/μl) for 20 minutes at 4° C. After 20 minutes, the whole 20 microlitres was mixed by pipetting up and down 10 times. A 4 microlitre aliquot of the cell-reagent mixture (comprising approximately 1000 cells and 2000 multimeric barcoding reagents) was transferred to a new PCR tube and 100 microlitres of cold lysis and indexing solution (comprising 100 millimolar Tris-HCl PH 7.5, 10 millimolar EDTA, 150 millimolar LiCl, 0.001% BRIJ58, 30% PEG 4000) was added and mixed by gently pipetting up and down 10 times. The PCR tube containing the cells and multimeric barcoding reagents diluted into lysis and indexing solution was placed on a thermal cycler and incubated at 65° C. for 30 seconds, 40° C. for 2 minutes, 35° C. for 3 minutes and then held at 20° C.

Following capture of the indexed mRNA using 4 microlitres of mRNA capture reagents, reverse transcription, second strand synthesis, PCR amplification and PCR for attachment of sequencing adapters was carried out as per Method 14 (“Assay for single cell whole transcriptome sequencing”) to generate libraries for sequencing.

Sequencing data was processed using a custom pipeline whereby reads were demultiplexed based on the barcode sequence to generate reads for single cells, and then mapped to a combined mouse-human custom genome. Mapped reads were then deduplicated and the uniquely mapped reads (UMR) for each species were plotted against the other.

### Analysis

FIG. 56 shows both duplicated and de-duplicated uniquely mapped reads for a human (Hsap; Homo Sapiens) and mouse (Mmus; Mus Musculus) species-mixed sequencing library.

In the context of both duplicated and de-duplicated reads, several multimeric barcoding reagents can be identified along or close to the horizontal or vertical axis, representing single mouse or human cells with a high number of uniquely mapped reads (UMR) that have been derived from messenger RNA barcoding by individual multimeric barcoding reagents. The data shown in these plots demonstrates that the analysis pipeline processes raw (non-de-duplicated) mapped reads and de-duplicates them to leave only a single UMR with a unique start and end site along the genome, whilst retaining high-fidelity single-cell data represented by the multimeric barcoding reagents plotted along or near to the x and y axes. The single cells that are successfully called with high confidence and such are labelled on the plots with the specific multimeric barcoding reagent set that was responsible for indexing each cell.

### FIG. 57

FIG. 57 illustrates the number of single-cells identified in the context of freeze-thaw versus without freeze-thaw. Flash freezing followed by rapid thawing of cells within lysis buffer increases the number of cells captured.

### FIG. 58

FIG. 58 shows the number of Homo Sapiens or Mus Musculus genes identified per cell in the context of freeze-thaw versus without freeze-thaw. Flash freezing followed by rapid thawing of cells within lysis buffer increases the number of genes captured per cell.

### FIG. 59

FIG. 59A&B shows Human/Mouse species mixing results with or without freeze-thaw in lysis buffer. Any cell containing at least 90% of genes from a single species and at least 50 nuclear genes from that species is identified as a single cell. In the freeze-thaw sample (B), more single-cells are identified than in the non-freeze-thaw sample (A). FIG. 59 C&D shows the total nuclear gene count from each single-cell. The freeze-thaw sample (D) identifies more total nuclear genes per cell than the non-freeze-thaw sample.

### FIG. 60

FIG. 60 shows the number of single-cells identified per sample. Increasing formamide concentration in the lysis buffer increases the number of cells captured. No additive in the lysis buffer (0% formamide) gives the lowest number of cells captured.

### FIG. 61

FIG. 61A&B shows Human/Mouse species mixing results with or without additive in lysis buffer. Any cell containing at least 90% of genes from a single species and at least 50 nuclear genes from that species is identified as a single cell. Lysis buffer containing 20% formamide (B) identifies more single-cells lysis buffer without additive (A). Lysis buffers containing 20% formamide reduces noise and provides the desired species bias. FIG. 61 C&D shows the total nuclear gene count from each single-cell. Lysis buffer containing 20% formamide (D) identifies more total nuclear genes per cells than lysis buffer without formamide.

### FIG. 62

FIG. 62A&B shows Human/Mouse species mixing results with or without additive in lysis buffer. Any cell containing at least 90% of genes from a single species and at least 50 nuclear genes from that species is identified as a single cell. Lysis buffer containing 20% Y-butyrolactone (B) identifies more single-cells lysis buffer without additive (A).

### FIG. 63A

In a single tube >500 high-quality single cells can be identified. Data down-sampled to 5 million reads for the sample shown in FIG. 69A. A total of 100 and 419 single cells passing thresholds (90% species purity and at least 100 nuclear genes observed) were identified for human (hsap) and mouse (mmus) cells respectively. Sample shown was studying the impact of additional MgCl₂ addition to a PCR step in the protocol.

Assay was performed using a cell mixture of HEK293 and NIH3T3 cells.

All cell handling steps performed with 0.1% BSA coated plasticware.

Cell-reagent binding: 10 μL of a 9,216 plex 14.5 μm reagent library (1,000 reagents/μL) was allowed to settle to the bottom of a PCR strip tube for 10 minutes and was followed by 10 μL cell mixture addition (500 cells/μL) and incubation for 20 minutes. Samples were disrupted (10 mixes with a using 15 μL volume) before sampling into assay tube. All performed at 4° C.

Index reaction conditions: 52 μL lysis reactions. 4 μL of disrupted settled cells and reagents sampled into 52 μL of lysis buffer containing target capture beads conjugated with a Poly-T oligo CO6. (No detergent, 30% PEG 4 k, 100 mM Tris HCl PH 7.5, 150 mM LiCl, 10 mM EDTA pH 8, 80 mM DTT, 15 nM R2′_endblock_invdT, 250 nM BO_L3, 20% Y-butyrolactone, 2 μl CO6 capture beads).

Lysis and indexing conditions: 15 min in dry ice, 4° C. hold, 65° C. for 30 s, 55° C. to 40° C. for 90 sec decreasing-(1° C./6 sec), 35° C. to 20° C. for 3 min decreasing-(1° C./9 sec), 20° C.—hold.

Capture Conditions: 50 μl of capture buffer used (100 mM Tris HCl PH 7.5, 150 mM LiCl, 10 mM EDTA pH 8, 80 mM DTT, 15 nM R2′_endblock_invdT, 50 nM BO_L3). Final captured volume was 102 μL. Capture performed for 5 minutes rotating at 20 RPM at room temperature.

### FIG. 63 B

In a single tube 448 high-quality single cells can be identified. Data down-sampled to 5 million reads for shown sample. A total of 160 and 288 single cells passing thresholds (90% species purity and at least 100 nuclear genes observed) were identified for human (hsap) and mouse (mmus) cells respectively. Sample shown had a 1000 cell input, demonstrating a cell capture rate of 44.8%. Sample shown was studying the removal of aliquot step from the protocol and an alternative settling technique.

Assay was performed using a cell mixture of JURKAT and NIH3T3 cells.

All cell handling steps performed with 0.1% BSA coated plasticware.

Cell-reagent binding: 10 μL of a 9,216 plex 14.5 μm reagent library (1,000 reagents/μL) was allowed to settle to the bottom of a PCR strip tube for 10 minutes and was followed by 20 μl of cell mixture addition (50 cells/μL) (total 1000 cells). Sample was gently centrifuged for 2 mins at 100 g, 20 μL of supernatant was removed, leaving 10 μL of bound cells and reagents). All performed at 4° C.

Index reaction conditions: 104 μL lysis reactions. 4 μL of settled cells and reagents sampled into 104 uL of lysis buffer containing target capture beads conjugated with a Poly-T oligo CO6. (No detergent, 30% PEG 4 k, 100 mM Tris HCl PH 7.5, 150 mM LiCl, 10 mM EDTA pH 8, 80 mM DTT 15 nM R2′_endblock_invdT, 250 nM BO_L3, 20% Y-butyrolactone, 4 uL CO6 capture beads).

Lysis and indexing conditions: frozen for 15 minutes in dry ice, then 4° C. hold, 65° C. for 30 s, 55° C. to 40° C.—for 90 sec decreasing-(1° C./6 sec), 35° C. to 20° C. for 3 min decreasing-(1° C./9 sec), 20° C.—hold.

Capture Conditions: 100 ul of capture buffer used (100 mM Tris HCl PH 7.5, 150 mM LiCl, 10 mM EDTA pH 8, 80 mM DTT, 15 nM R2′_endblock_invdT, 50 nM BO_L3). Final captured volume was 204 uL. Capture performed for 5 minutes rotating at 20 RPM at room temperature.

Analysis/Results: For both experiments high cell output was achieved.

### FIG. 64A

In a single tube high transcript number single cells can be identified. Data down-sampled to 5 million reads for shown sample. Sample shown was studying post-capture wash buffers and volume used.

Assay was performed using a cell mixture of HEK293 and NIH3T3 cells.

All cell handling steps performed with 0.1% BSA coated plasticware.

Cell-reagent binding: 10 μL of a 9,216 plex 14.5 μm reagent library (1,000 reagents/L) was allowed to settle to the bottom of a PCR strip tube for 10 minutes and was followed by 10 μL cell mixture addition (500 cells/μL) and incubation for 20 minutes. Samples were disrupted (10 mixes with a using 15 μL volume) before sampling into assay tube. All performed at 4° C.

Index reaction conditions: 52 μL lysis reactions. 4 μL of disrupted settled cells and reagents sampled into 52 μL of lysis buffer containing target capture beads conjugated with a Poly-T oligo CO6. (No detergent, 30% PEG 4 k, 100 mM Tris HCl PH 7.5, 150 mM LiCl, 10 mM EDTA pH 8, 80 mM DTT, 15 nM R2′_endblock_invdT, 250 nM BO_L3, 20% Y-butyrolactone, 2 μl CO6 capture beads).

Lysis and indexing conditions: frozen for 15 minutes in dry ice, then 4° C. hold, 65° C. for 30 s, 55° C. to 40° C.—for 90 sec decreasing-(1° C./6 sec), 35° C. to 20° C. for 3 min decreasing-(1° C./9 sec), 20° C.—hold.

Capture Conditions: 50 μl of capture buffer used (100 mM Tris HCl PH 7.5, 150 mM LiCl, 10 mM EDTA pH 8, 80 mM DTT, 15 nM R2′_endblock_invdT, 50 nM BO_L3). Final captured volume was 102 μL. Capture performed for 5 minutes rotating at 20 RPM at room temperature.

### FIG. 64 B

In a single tube, single-cells with >2000 nuclear genes are observed for both human and mouse cell lines. Data down-sampled to 5 million reads for shown sample. The maximum observed nuclear genes observed in this sample was 3081 nuclear genes.

Assay was performed using a cell mixture of HEK293 and NIH3T3 cells.

All cell handling steps performed with 0.1% BSA coated plasticware.

Cell-reagent binding: 10 μL of a 9,216 plex 14.5 μm reagent library (1,000 reagents/μL) was allowed to settle to the bottom of a PCR strip tube for 10 minutes and was followed by 10 μL cell mixture addition (500 cells/μL) and incubation for 20 minutes. Samples were disrupted (10 mixes with a using 15 μL volume) before sampling into assay tube. All performed at 4° C.

Index reaction conditions: 52 μL lysis reactions. 4 μL of disrupted settled cells and reagents sampled into 52 μL of lysis buffer containing target capture beads conjugated with a Poly-T oligo CO6. (No detergent, 30% PEG 4 k, 100 mM Tris HCl PH 7.5, 150 mM LiCl, 10 mM EDTA pH 8, 80 mM DTT, 15 nM R2′_endblock_invdT, 250 nM BO_L3, 20% Y-butyrolactone, 2 ul CO6 capture beads).

Lysis and indexing conditions: 15 min in dry ice, 4° C. hold, 65° C. for 30 s, 55° C. to 40° C.—for 90 sec decreasing-(1° C./6 sec), 35° C. to 20° C. for 3 min decreasing-(1° C./9 sec), 20° C.—hold.

Capture Conditions: 50 ul of capture buffer used (100 mM Tris HCl PH 7.5, 150 mM LiCl, 10 mM EDTA pH 8, 80 mM DTT, 15 nM R2′_endblock_invdT, 50 nM BO_L3). Final captured volume was 102 uL. Capture performed for 5 minutes rotating at 20 RPM at room temperature.

Analysis/Results: For both experiments high transcript and gene numbers were achieved.

### FIG. 65A

In a single tube high transcript number single cells can be identified. Deep sequencing performed for selected sample. Sample shown was studying increased R2′_endblock_invdT and BO_L3 concentrations in the lysis buffer.

Assay was performed using a cell mixture of JURKAT and NIH3T3 cells.

All cell handling steps performed with 0.1% BSA coated plasticware.

Cell-reagent binding: 10 μL of a 9,216 plex 14.5 μm reagent library (1,000 reagents/μL) was allowed to settle to the bottom of a PCR strip tube for 10 minutes and was followed by 10 μL cell mixture addition (500 cells/μL) and incubation for 20 minutes. Samples were disrupted (10 mixes with a using 15 μL volume) before sampling into assay tube. All performed at 4° C.

Index reaction conditions: 104 μL lysis reactions. 4 μL of disrupted settled cells and reagents sampled into 104 μL of lysis buffer containing 8 μL target capture beads conjugated with a Poly-T oligo CO6. (0.001% Brij 58, 30% PEG 4 k, 100 mM Tris HCl PH 7.5, 150 mM LiCl, 10 mM EDTA pH 8, 80 mM DTT, 75 nM R2′_endblock_invdT, 250 nM BO_L3, 20% Y-butyrolactone, 8 μl CO6 capture beads).

Lysis and indexing conditions: 15 minutes in dry ice, 4° C. hold, 65° C. for 30 s, 55° C. to 40° C.—for 90 sec decreasing-(1° C./6 sec), 35° C. to 20° C. for 3 min decreasing-(1° C./9 sec), 20° C.—hold.

Capture Conditions: 100 μl of capture buffer used (100 mM Tris HCl PH 7.5, 150 mM LiCl, 10 mM EDTA pH 8, 80 mM DTT, 75 nM R2′_endblock_invdT, 250 nM BO_L3). Final captured volume was 204 μL. Capture performed for 5 minutes rotating at 20 RPM at room temperature.

### FIG. 65 B

In a single tube, single-cells with >2000 nuclear genes are observed for both human and mouse cell lines. Deep sequencing performed for selected sample. Sample shown was studying increased R2′_endblock_invdT and BO_L3 concentrations in the lysis buffer. The maximum observed nuclear genes observed in this sample was >4000 nuclear genes.

Assay was performed using a cell mixture of JURKAT and NIH3T3 cells.

All cell handling steps performed with 0.1% BSA coated plasticware.

Cell-reagent binding: 10 μL of a 9,216 plex 14.5 μm reagent library (1,000 reagents/μL) was allowed to settle to the bottom of a PCR strip tube for 10 minutes and was followed by 10 μL cell mixture addition (500 cells/μL) and incubation for 20 minutes. Samples were disrupted (10 mixes with a using 15 μL volume) before sampling into assay tube. All performed at 4° C.

Index reaction conditions: 104 μL lysis reactions. 4 μL of disrupted settled cells and reagents sampled into 104 μL of lysis buffer containing 8 μL target capture beads conjugated with a Poly-T oligo CO6. (0.001% Brij 58, 30% PEG 4 k, 100 mM Tris HCl pH 7.5, 150 mM LiCl, 10 mM EDTA pH 8, 80 mM DTT, 75 nM R2′_endblock_invdT, 250 nM BO_L3, 20% Y-butyrolactone, 8 μl CO6 capture beads).

Lysis and indexing conditions: 15 minutes in dry ice, 4° C. hold, 65° C. for 30 s, 55° C. to 40° C.—for 90 sec decreasing-(1° C./6 sec), 35° C. to 20° C. for 3 min decreasing-(1° C./9 sec), 20° C.—hold.

Capture Conditions: 100 μl of capture buffer used (100 mM Tris HCl PH 7.5, 150 mM LiCl, 10 mM EDTA pH 8, 80 mM DTT, 75 nM R2′_endblock_invdT, 250 nM BO_L3). Final captured volume was 204 uL. Capture performed for 5 minutes rotating at 20 RPM at room temperature.

Analysis/Results: For both experiments high transcript and gene numbers were achieved by deep sequencing.

### Sequences of Oligonucleotides Mentioned Above in Relation to FIGS. 63A, 63B, 64A, 64B, 65A and 65B:

BO_L3:
5′ AAAAAAAAAAAAAAAAAAAAAAAAAA/3InvdT/3′
R2′_endblock_invdT:
5′ GTGCTCTTCCGATCT/3InvdT/3′

(where 3InvdT is a 3′ Inverted dT modified base)

1. A method of preparing a nucleic acid sample for sequencing, wherein the sample comprises at least 2 cells, and wherein the method comprises in order the steps of:
(a) contacting the sample with a library comprising at least two multimeric barcoding reagents, wherein each multimeric barcoding reagent comprises first and second barcoded oligonucleotides linked together, wherein the barcoded oligonucleotides each comprise a barcode region and wherein the barcode regions of the first and second barcoded oligonucleotides of a first multimeric barcoding reagent of the library are different to the barcode regions of the first and second barcoded oligonucleotides of a second multimeric barcoding reagent of the library;
(b) lysing the cells or permeabilizing the cell membranes of the cells; and
(c) appending the first and second barcoded oligonucleotides of the first multimeric barcoding reagent to first and second sub-sequences of a target nucleic acid of the first cell to produce first and second barcoded target nucleic acid molecules, and appending the first and second barcoded oligonucleotides from the second multimeric barcoding reagent to first and second sub-sequences of a target nucleic acid of the second cell to produce first and second barcoded target nucleic acid molecules;
wherein the method further comprises (i) freezing the cells and, optionally, (ii) thawing the cells.

2. The method of claim 1,
wherein, in step (a), the first multimeric barcoding reagent binds to the cell membrane of a first cell prior to step (b), and wherein the second multimeric barcoding reagent binds to the cell membrane of a second cell prior to step (b).

3. The method of claim 1, wherein, in step (a),
each multimeric barcoding reagent comprises first and second barcoded oligonucleotides linked together and a cell-binding moiety, wherein the cell-binding moiety of the first multimeric barcoding reagent binds to the cell membrane of a first cell prior to step (b), and wherein the cell-binding moiety of the second multimeric barcoding reagent binds to the cell membrane of a second cell prior to step (b).

4. A method of preparing a nucleic acid sample for sequencing, wherein the sample comprises at least 2 cells, and wherein the method comprises in order the steps of:
(a) contacting the sample with a library comprising at least two multimeric barcoding reagents, wherein each multimeric barcoding reagent comprises
(i) a support,
(ii) at least two multimeric hybridization molecules, wherein each multimeric hybridization molecule is independently linked to the support and wherein each multimeric hybridization molecule comprises at least two hybridization molecules linked together, wherein each of the hybridization molecules comprises a nucleic acid sequence comprising a hybridization region, and
(iii) at least two barcoded oligonucleotides annealed to each of the multimeric hybridization molecules, wherein each barcoded oligonucleotide is annealed to one of the hybridization regions and wherein each barcoded oligonucleotide comprises a barcode region,
and wherein the barcode regions of the barcoded oligonucleotides of a first multimeric barcoding reagent of the library are different to the barcode regions of the barcoded oligonucleotides of a second multimeric barcoding reagent of the library;
(b) lysing the cells or permeabilizing the cell membranes of the cells; and
(c) (separately) appending each of the barcoded oligonucleotides of the first multimeric barcoding reagent to at least four sub-sequences of a target nucleic acid of the first cell to produce at least four barcoded target nucleic acid molecules, and (separately) appending each of the barcoded oligonucleotides of the second multimeric barcoding reagent to a least four sub-sequences of a target nucleic acid of the second cell to produce at least four barcoded target nucleic acid molecules;
wherein the method further comprises (i) freezing the cells and, optionally, (ii) thawing the cells.

5. The method of claim 4,
wherein, in step (a) (iii), the first multimeric barcoding reagent binds to the cell membrane of a first cell prior to step (b), and wherein the second multimeric barcoding reagent binds to the cell membrane of a second cell prior to step (b).

6. A method of preparing a nucleic acid sample for sequencing, wherein the sample comprises at least 2 cells, and wherein the method comprises in order the steps of:
(a) contacting the sample with a library comprising at least two multimeric barcoding reagents, wherein each multimeric barcoding reagent comprises
(i) a support,
(ii) at least two multimeric hybridization molecules, wherein each multimeric hybridization molecule is independently linked to the support and wherein each multimeric hybridization molecule comprises at least two hybridization molecules linked together, wherein each of the hybridization molecules comprises a nucleic acid sequence comprising a hybridization region,
(iii) at least two barcoded oligonucleotides annealed to each of the multimeric hybridization molecules, wherein each barcoded oligonucleotide is annealed to one of the hybridization regions and wherein each barcoded oligonucleotide comprises a barcode region, and
(iv) a cell-binding moiety linked to each multimeric hybridization molecule,
and wherein the barcode regions of the barcoded oligonucleotides of a first multimeric barcoding reagent of the library are different to the barcode regions of the barcoded oligonucleotides of a second multimeric barcoding reagent of the library, wherein the cell-binding moiety of the first multimeric barcoding reagent binds to the cell membrane of a first cell prior to step (b), and wherein the cell-binding moiety of the second multimeric barcoding reagent binds to the cell membrane of a second cell prior to step (b);
(b) lysing the cells or permeabilizing the cell membranes of the cells; and
(c) (separately) appending each of the barcoded oligonucleotides of the first multimeric barcoding reagent to at least four sub-sequences of a target nucleic acid of the first cell to produce at least four barcoded target nucleic acid molecules, and (separately) appending each of the barcoded oligonucleotides of the second multimeric barcoding reagent to a least four sub-sequences of a target nucleic acid of the second cell to produce at least four barcoded target nucleic acid molecules;
wherein the method further comprises (i) freezing the cells and, optionally, (ii) thawing the cells.

7. The method of claim 1, wherein the step of (i) freezing the cells, and, optionally, (ii) thawing the cells, is/are performed after step (a) and, optionally, prior to step (c).

8. The method of claim 1, wherein the step of lysing the cells or permeabilizing the cell membranes of the cells comprises (i) freezing the cells, and, optionally, (ii) thawing the cells.

9. The method of claim 1, wherein the cells are comprised within a single contiguous aqueous volume during steps (a), (b) and/or (c).

10. The method of claim 1, wherein the step of freezing is performed at a temperature of less than −20° C., less than −30° C., less than −40° C., less than-50° C., less than −50° C., less than −60° C., less than −70° C., less than −75° C. or less than −80° C.

11. The method of claim 1, wherein following the step of freezing the cells are maintained in a frozen state for at least 1 minute, at least 5 minutes, at least 10 minutes, at least 30 minutes, at least 1 hour, at least 1 day, at least 3 days, at least 7 days, at least 1 month, at least 6 months or at least 1 year.

12. The method of claim 1, wherein the step of thawing the cells is carried out at at least 4° C., at least 10° C., at least 20° C., at least 25° C., at least 30° C., at least 37° C., at least 40° C., at least 45° C., at least 50° C., at least 55° C., at least 60° C., at least 65° C., at least 70° C., at least 75° C., or at least 80° C.

13. The method of claim 1, wherein the step of thawing is carried out for at least 5 seconds, at least 10 seconds, at least 30 seconds, at least 1 minute, or at least 5 minutes.

14. The method of claim 1, wherein the method further comprises (d) capturing the barcoded oligonucleotides and/or barcoded target nucleic acid molecules and/or multimeric barcoding reagents on a solid support.

15. The method of claim 14, wherein the target nucleic acids are mRNA and wherein step (d) comprises capturing barcoded oligonucleotides appended to sub-sequences of mRNA, and wherein the method further comprises (e) reverse transcription of mRNA to generate cDNA.

16. The method of claim 14, wherein the solid support is beads.

17. The method of claim 14, wherein the solid support comprises streptavidin moieties and the barcoded oligonucleotides and/or barcoded target nucleic acid molecules and/or multimeric barcoding reagents are captured on the solid support through streptavidin-biotin interaction.

18. The method of claim 14, wherein the method comprises contacting the sample with the solid support in step (a), (b), and/or (c).

19. The method of claim 1, wherein step (a), (b), and/or (c) is/are performed in the presence of an RNA stabilising molecule.

20. The method of claim 1, wherein step (a), (b), and/or (c) is/are performed in the presence of a protic or aprotic solvent.

21. The method of claim 1, wherein step (a), (b), and/or (c) is/are performed in the presence of a molecular crowding agent.

22. The method of claim 1, wherein step (a), (b), and/or (c) is/are performed in a high-viscosity solution.

23. The method of claim 1, wherein the method is for preparing first and second nucleic acid samples for sequencing, wherein each sample comprises at least 2 cells, and wherein the method comprises performing for each sample steps (a), (b) and (c).

24. The method of claim 23, wherein step (a) is performed at a different timepoint for the first and second nucleic acid samples.

25. The method of claim 23, wherein the step of freezing the cells is performed at different timepoints for the first and second nucleic acid samples.

26. The method of claim 23, wherein the cells of the first nucleic acid sample are maintained in a frozen state for a different duration of time relative to the duration of time for which the cells of the second nucleic acid sample are maintained in a frozen state.

27. The method of claim 26, wherein the difference between the duration of time for which the cells of the first nucleic acid sample are maintained in a frozen state and the duration of time for which the cells of the second nucleic acid sample are maintained in a frozen state is at least 5 minutes, at least 30 minutes, at least 1 hour, at least 6 hours, at least 12 hours, at least 24 hours, at least 7 days, at least 1 month, at least 6 months or at least 1 year.

28. The method of claim 23, wherein step (c) is performed within a single contiguous 24-hour period for both the first and second nucleic acid samples.
